(19) United States
(12) Patent Application Publication
(10) Pub. No.: US 2010/0216788 A1
(43) Pub. Date: Aug. 26, 2010

(54) FUSED HETEROCYCLIC COMPOUND

(75) Inventors: Tomoyasu Ishikawa, Tsukuba-shi (JP); Kazuhiro Miwa, Osaka-shi (JP); Masaki Seto, Osaka-shi (JP); Hiroshi Banno, Tsukuba-shi (JP); Youichi Kawakita, Tsukuba-shi (JP)

Correspondence Address: HAMRE, SCHUMANN, MUELLER & LARSON, P.C., P.O. BOX 2902, MINNEAPOLIS, MN, 55402-0902, US

(73) Assignee: TAKEDA PHARMACEUTICAL COMPANY LIMITED, Osaka-shi, Osaka (JP)

(21) Appl. No.: 12/095,543

(22) PCT Filed: Dec. 1, 2006
(86) PCT No.: PCT/JP2006/324499

§ 371 Date: May 30, 2008

(30) Foreign Application Priority Data
Dec. 2, 2005 (JP) 2005-349858
Mar. 7, 2006 (JP) 2006-060648

(51) Int. Cl. A61K 31/5377; C07D 487/04; A61K 31/519; C07D 413/14; A61P 35/00

(52) U.S. Cl. 514/234.2; 544/280; 514/265.1; 544/118

(57) ABSTRACT

The present invention provides a compound represented by the formula:

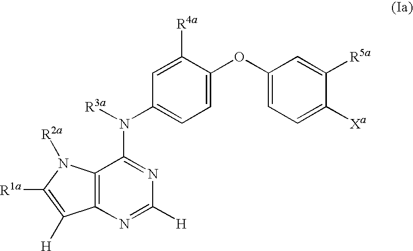

wherein
R^{1a} is a hydrogen atom,
R^{2a} is a C₁₋₆ alkyl group substituted by a group represented by —NR^{6a}—CO—(CH₂)ₙ—SO₂— optionally halogenated C₁₋₄ alkyl
wherein n is an integer of 1 to 4, R^{6a} is a hydrogen atom or a C₁₋₄ alkyl group, and —(CH₂)ₙ— is optionally substituted by C₁₋₄ alkyl,
R^{3a} is a hydrogen atom or a C₁₋₆ alkyl group,
R^{4a} is a halogen atom or a C₁₋₆ alkyl group,
R^{5a} is a halogen atom or a C₁₋₆ alkyl group, and
X^{a} is a hydrogen atom or a halogen atom,
or a salt thereof.

The compound of the present invention has a superior tyrosine kinase inhibitory action, is highly safe, and is sufficiently satisfactory as a pharmaceutical product.

### TECHNICAL FIELD

The present invention relates to a fused pyrimidine compound having a growth factor receptor tyrosine kinase inhibitory activity, which is useful for the prophylaxis or treatment of cancer, a production method thereof and use thereof.

### BACKGROUND OF THE INVENTION

The gene of cell growth factor and growth factor receptor is called a protooncogene and plays a key role in the pathology of human tumor. The epithelial cell growth factor receptor family (erbB) includes EGFR, HER2, HER3 and HER4, which are type I receptor type tyrosine kinases. These erbB family express in various cell groups, and are deeply involved in the control of the growth and differentiation of cells and the control of suppression of cell death (apoptosis suppression). For example, high expression of EGFR and HER2, and homeostatic activation of receptors are empirically known to transform cells.

It is also known that high expression and simultaneous expression of each of these receptors are poor prognostic factors in various cancer patients.

These receptors are bound with many peptide ligands such as EGF, TGF_{α} and the like, and binding of the ligand promotes homo- or heterodimerization of the receptors. This induces increase of kinase activity from self-phosphorylation or transphosphorylation of the receptors, and causes activation of downstream signaling pathway (MAPK, Akt) via a protein bound with a particular phosphorylated tyrosine residue. This is the mechanism of the receptor activity of the above-mentioned cell growth, differentiation, cell death suppression and the like, which is considered to be responsible for the high expression of receptor in cancer and malignant degeneration of cancer due to topical increase in the ligand concentration.

Many cancers are associated with the high expression of EGFR or HER2. For example, breast cancer (20-30%), ovarian cancer (20-40%), non-small cell lung cancer (30-60%), colorectal cancer (40-80%), prostate cancer (10-60%), bladder cancer (30-60%), kidney cancer (20-40%) and the like can be mentioned. Moreover, receptor expression and prognosis are correlated, and receptor expression is a poor proghostic factor in breast cancer, non-small cell lung cancer and the like.

In recent years, clinical use of a humanized anti-HER2 antibody (Trastuzumab) against HER2 highly expressing breast cancer, clinical trial of anti-EGFR antibody and clinical trials of several low molecular weight receptor enzyme inhibitors have demonstrated a potential of these drugs against HER2 or EGFR for therapeutic drugs for cancer. While these drugs show a tumor growth inhibitory action in clinical and non-clinical trials, they are known to induce inhibition of receptor enzyme activity and suppression of downstream signaling pathway. Therefore, a compound inhibiting EGFR or HER2 kinase, or inhibiting activation of EGFR or HER2 kinase is effective as a therapeutic drug for cancer.

As a compound that inhibits receptor type tyrosine kinases represented by HER2/EGFR kinase, fused heterocyclic compounds (e.g., WO97/13771, WO98/02437, WO00/44728), quinazoline derivatives (e.g., WO02/02552, WO01/98277, WO03/049740, WO03/050108), thienopyrimidine derivatives (e.g., WO03/053446), aromatic azole derivatives (e.g., WO98/03648, WO01/77107, WO03/031442) and the like are known; however, there is no HER2 kinase inhibitory substance to the present that has been marketed as a therapeutic drug for cancer.

As to pyrrolo[3,2-d]pyrimidine derivatives, the following compounds are known as compounds having a cell growth inhibitory activity (Khim.-Farm. Zh., 1982, 16, 1338-1343; Collect. Czech. Chem. Commun., 2003, 68, 779-791).

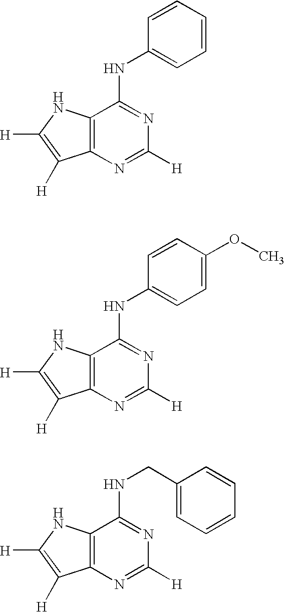

As a compound having a receptor type tyrosine kinase inhibitory activity, the following pyrrolo[3,2-d]pyrimidine derivative is known (WO96/40142, WO98/23613).

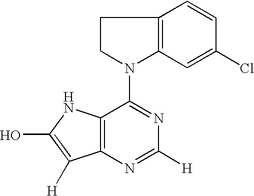

Furthermore, as to pyrazolo[4,3-d]pyrimidine derivatives, 3,5,7-trisubstituted pyrazolo[4,3-d]pyrimidine derivatives are known as compounds having a CDK inhibitory action, a cell growth inhibitory action and/or an apoptosis inducing action (EP-A-1348707), and 3-isopropylpyrazolo[4,3-d]pyrimidine derivatives are known as compounds having a CDK1/cyclin B inhibitory activity (Bioorganic & Medicinal Chemistry Letters, 2003, 13, 2989-2992). Furthermore, synthesis of 3-methylpyrazolo[4,3-d]pyrimidine derivatives has been reported (The Journal of Organic Chemistry, 1956, 21, 833-836).

### DISCLOSURE OF THE INVENTION

The present invention aims at providing a compound having a superior tyrosine kinase inhibitory action, which is low toxic and highly safe as a pharmaceutical product.

The present inventors have conducted intensive studies in an attempt to solve the aforementioned problems and found that the compounds represented by the following formulas (Ia)-(Ih) and salts thereof have a superior tyrosine kinase inhibitory action. Further studies have resulted in the completion of the present invention.

Accordingly, the present invention relates to the following.
[1a] A compound represented by the formula:

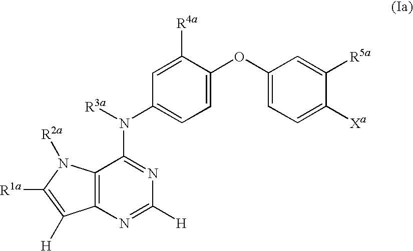

wherein
R^{1a} is a hydrogen atom,
R^{2a} is a C₁₋₆ alkyl group substituted by a group represented by —NR^{6a}—CO—(CH₂)ₙ—SO₂-optionally halogenated C₁₋₄ alkyl
wherein n is an integer of 1 to 4, R^{6a} is a hydrogen atom or a C₁₋₄ alkyl group, and —(CH₂)ₙ— is optionally substituted by C₁₋₄ alkyl,
R^{3a} is a hydrogen atom or a C₁₋₆ alkyl group,
R^{4a} is a halogen atom or a C₁₋₆ alkyl group,
R^{5a} is a halogen atom or a C₁₋₆ alkyl group, and
X^{a} is a hydrogen atom or a halogen atom,
or a salt thereof,
provided that N-[2-(4-{[3-chloro-4-(3-chlorophenoxy)phenyl]amino}-5H-pyrrolo[3,2-d]pyrimidin-5-yl)ethyl]-2-(methylsulfonyl)acetamide is excluded.
[2a] The compound of the above-mentioned [1a], wherein X^{a} is a hydrogen atom.
[3a] The compound of the above-mentioned [2a], wherein R^{1a} is a hydrogen atom,
R^{2a} is a C₁₋₆ alkyl group substituted by a group represented by —NR^{6aa}—CO—CR^{7a}R^{8a}—SO₂—C₁₋₄ alkyl
wherein R^{6aa} is a hydrogen atom or a methyl group, R^{7a} and R^{8a} are the same or different and each is a hydrogen atom or a methyl group,
R^{3a} is a hydrogen atom,
R^{4a} is a chlorine atom or a methyl group, and
R^{5a} is a fluorine atom, a chlorine atom or a methyl group.
[4a] The compound of the above-mentioned [3a], wherein R^{7a} and R^{8a} are methyl groups.
[5a] A compound selected from the following:
N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide,
N-[2-(4-{[3-chloro-4-(3-chlorophenoxy)phenyl]amino}-5H-pyrrolo[3,2-d]pyrimidin-5-yl)ethyl]-2-(ethylsulfonyl)acetamide,
N-[2-(4-{[3-chloro-4-(3-chlorophenoxy)phenyl]amino}-5H-pyrrolo[3,2-d]pyrimidin-5-yl)ethyl]-N,2-dimethyl-2-(methylsulfonyl)propanamide,
N-[2-(4-{[3-chloro-4-(3-methylphenoxy)phenyl]amino}-5H-pyrrolo[3,2-d]pyrimidin-5-yl)ethyl]-2-(methylsulfonyl)acetamide,
N-[2-(4-{[3-chloro-4-(3-fluorophenoxy)phenyl]amino}-5H-pyrrolo[3,2-d]pyrimidin-5-yl)ethyl]-2-(methylsulfonyl)acetamide, and
N-[2-(4-{[4-(3-chlorophenoxy)-3-methylphenyl]amino}-5H-pyrrolo[3,2-d]pyrimidin-5-yl)ethyl]-2-methyl-2-(methylsulfonyl)propanamide,
or a salt thereof, or a hydrate thereof.
[6a] A prodrug of the compound of the above-mentioned [1a].
[7a] A production method of the compound of the above-mentioned [1a] or a salt thereof, which comprises reacting a compound represented by the formula:

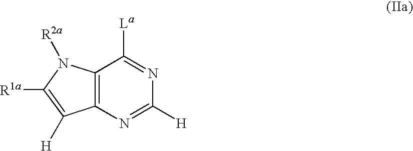

wherein L^{a} is a leaving group, and the other symbols are as defined above,
or a salt thereof and a compound represented by the formula:

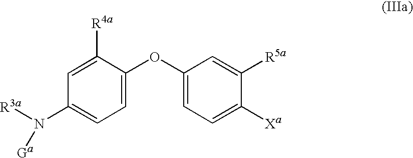

wherein G^{a} is a hydrogen atom or a metal atom, and the other symbols are as defined above,
or a salt thereof.
[8a] A pharmaceutical agent comprising the compound of the above-mentioned [1a] or a salt thereof, or a prodrug thereof.
[9a] The pharmaceutical agent of the above-mentioned [8a], which is a tyrosine kinase inhibitor.
[10a] The pharmaceutical agent of the above-mentioned [8a], which is an agent for the prophylaxis or treatment of cancer.
[11a] The pharmaceutical agent of the above-mentioned [10a], wherein the cancer is breast cancer, ovarian cancer, colorectal cancer, gastric cancer, esophagus cancer, prostate cancer, lung cancer, pancreatic cancer or kidney cancer.
[12a] A method for the prophylaxis or treatment of cancer in a mammal, which comprises administering an effective amount of the compound of the above-mentioned [1a] or a salt thereof, or a prodrug thereof, to the mammal.
[13a] Use of the compound of the above-mentioned [1a] or a salt thereof, or a prodrug thereof, for the production of an agent for the prophylaxis or treatment of cancer.
[1b] A compound represented by the formula:

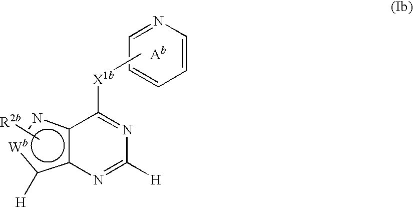

wherein
W^{b} is C(R^{1b}) or N,
ring A^{b} is an optionally substituted pyridine ring,
X^{1b} is —NR^{3b}—Y^{1b}—, —O—, —S—, —SO—, —SO₂— or —CHR^{3b}—
wherein R^{3b} is a hydrogen atom or an optionally substituted aliphatic hydrocarbon group, or R^{3b} is optionally bonded to the carbon atom on the pyridine ring for ring A^{b} to form an optionally substituted ring structure, and Y^{1b} is a bond, or a C₁₋₄ alkylene or —O—(C₁₋₄ alkylene)-, each of which is optionally substituted, and
R^{1b} is a hydrogen atom, a halogen atom, or an optionally substituted group bonded via a carbon atom, a nitrogen atom or an oxygen atom,
R^{2b} is a hydrogen atom, or an optionally substituted group bonded via a carbon atom or a sulfur atom, or R^{1b} and R^{2b}, or R^{2b} and R^{3b} are optionally bonded to form an optionally substituted ring structure,
or a salt thereof.
[2b] The compound of the above-mentioned [1b], which is a compound represented by the formula:

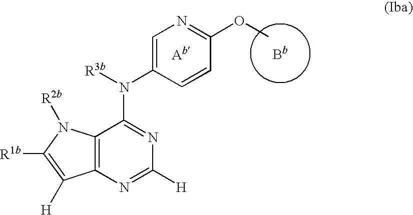

wherein ring A^{b′} is an optionally further substituted pyridine ring, ring B^{b} is an optionally substituted C₆₋₁₄ aryl group, and the other symbols are as defined above.
[3b] The compound of the above-mentioned [2b], wherein R^{1b} is a hydrogen atom, a halogen atom, a cyano group or an optionally halogenated C₁₋₆ alkyl group,
R^{2b} is a C₁₋₆ alkyl group substituted by substituent(s) selected from the group consisting of
(i) —NR^{6ba}—CO—(CH₂)ₙ₁—SO₂—C₁₋₄ alkyl
wherein R^{6ba} is a hydrogen atom or a methyl group, n1 is an integer of 1 to 4, and —(CH₂)ₙ₁— is optionally substituted by C₁₋₄ alkyl,
(ii) —NR^{6bb}—CO—(CH₂)ₙ₂—OH
wherein R^{6bb} is a hydrogen atom or a methyl group, n2 is an integer of 1 to 4, and —(CH₂)ₙ₂— is optionally substituted by C₁₋₄ alkyl,
(iii) —O—(CH₂)ₙ₃13 OH
wherein n3 is an integer of 1 to 4, and —(CH₂)ₙ₃— is optionally substituted by C₁₋₄ alkyl, and
(iv) hydroxy,
R^{3b} is a hydrogen atom,
ring A^{b′} is a pyridine ring optionally substituted by substituent(s) selected from the group consisting of halogen and methyl, and
ring B^{b} is a phenyl group optionally substituted by substituent(s) selected from the group consisting of optionally halogenated C₁₋₆ alkyl, optionally halogenated C₁₋₆ alkoxy, C₁₋₆ alkyl-carbamoyl and halogen.
[4b] The compound of the above-mentioned [2b], wherein ring. A^{b′} is a pyridine ring optionally substituted by halogen, and
ring B^{b} is a phenyl group optionally substituted at the 3-position by substituent(s) selected from the group consisting of optionally halogenated C₁₋₆ alkyl, optionally halogenated C₁₋₆ alkoxy, C₁₋₆ alkyl-carbamoyl and halogen.
[5b] A compound selected from the following:
2-{2-[4-({5-chloro-6-[3-(trifluoromethyl)phenoxy]pyridin-3-yl}amino)-5H-pyrrolo[3,2-d]pyrimidin-5-yl]ethoxy}ethanol,
N-{2-[4-({5-chloro-6-[3-(trifluoromethyl)phenoxy]pyridin-3-yl}amino)-5H-pyrrolo[3,2-d]pyrimidin-5-yl]ethyl}-2-(methylsulfonyl)acetamide,
N-{2-[4-({5-chloro-6-[3-(trifluoromethyl)phenoxy]pyridin-3-yl}amino)-5H-pyrrolo[3,2-d]pyrimidin-5-yl]ethyl}-3-hydroxy-3-methylbutanamide,
N-{2-[4-({5-chloro-6-[3-(trifluoromethoxy)phenoxy]pyridin-3-yl}amino)-5H-pyrrolo[3,2-d]pyrimidin-5-yl]ethyl}-2-(methylsulfonyl)acetamide, and
N-(tert-butyl)-3-[(3-chloro-5-{[5-(2-hydroxyethyl)-5H-pyrrolo[3,2-d]pyrimidin-4-yl]amino}pyridin-2-yl)oxy]benzamide,
or a salt thereof.
[6b] A prodrug of the compound of the above-mentioned [1b].
[7b] A production method of the compound of the above-mentioned [1b] or a salt thereof, which comprises reacting a compound represented by the formula:

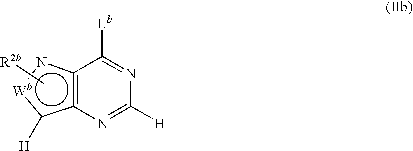

wherein L^{b} is a leaving group, and the other symbols are as defined above,
or a salt thereof and a compound represented by the formula:

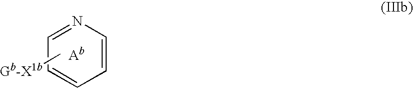

wherein G^{b} is a hydrogen atom or a metal atom, and the other symbols are as defined above,
or a salt thereof.
[8b] A pharmaceutical agent comprising the compound of the above-mentioned [1b] or a salt thereof, or a prodrug thereof.
[9b] The pharmaceutical agent of the above-mentioned [8b], which is a tyrosine kinase inhibitor.
[10b] The pharmaceutical agent of the above-mentioned [8b], which is an agent for the prophylaxis or treatment of cancer.
[11b] The pharmaceutical agent of the above-mentioned [10b], wherein the cancer is breast cancer, ovarian cancer, colorectal cancer, gastric cancer, esophagus cancer, prostate cancer, lung cancer, pancreatic cancer or kidney cancer.
[12b] A method for the prophylaxis or treatment of cancer in a mammal, which comprises administering an effective amount of the compound of the above-mentioned [1b] or a salt thereof, or a prodrug thereof, to the mammal.
[13b] Use of the compound of the above-mentioned [1b] or a salt thereof, or a prodrug thereof, for the production of an agent for the prophylaxis or treatment of cancer.
[14b] The compound of the above-mentioned [1b], which is a compound represented by the formula:

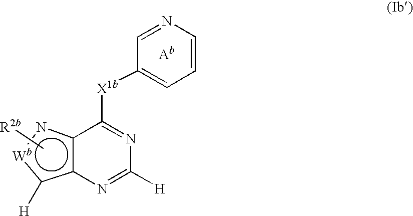

wherein each symbol is as defined above.
[1c] A compound represented by the formula:

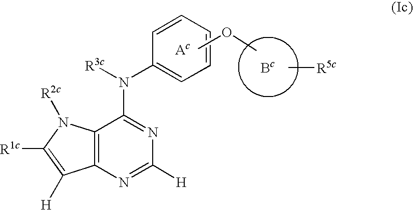

wherein
R^{1c} is a hydrogen atom, or an optionally substituted group bonded via a carbon atom, a nitrogen atom or an oxygen atom,
R^{2c} is an optionally substituted group bonded via a carbon atom or a sulfur atom, or
R^{1c} and R^{2c}, or R^{2c} and R^{3c} are optionally bonded to form an optionally substituted ring structure,
R^{3c} is a hydrogen atom or an optionally substituted aliphatic hydrocarbon group, or R^{3c} is optionally bonded to the carbon atom on the adjacent benzene ring to form an optionally substituted ring structure,
ring A^{c} is an optionally substituted benzene ring,
R^{5c} is
(i) an optionally substituted amino group,
(ii) an optionally substituted carbamoyl group,
(iii) an optionally substituted ureido group,
(iv) an optionally substituted sulfamoyl group,
(v) an optionally substituted heterocyclic group,
(vi) an optionally substituted C₂₋₆ alkoxy group
(vii) an optionally substituted aminomethyl group,
(viii) an optionally substituted carbamoylmethyl group,
(ix) an optionally substituted alkylsulfonyl group, or
(x) a cyano group, and
ring B^{c} is a C₆₋₁₄ aryl group or a C₅₋₈ cycloalkyl group, each of which is optionally further substituted besides R^{5c},
or a salt thereof,
provided that
N-(tert-butyl)-4-[2-chloro-4-({5-[2-(2-hydroxyethoxy)ethyl]-5H-pyrrolo[3,2-d]pyrimidin-4-yl}amino)phenoxy]benzamide hydrochloride,
4-[2-chloro-4-({5-[2-(2-hydroxyethoxy)ethyl]-5H-pyrrolo[3,2-d]pyrimidin-4-yl}amino)phenoxy]-N-(2,2-dimethylpropyl)benzamide,
3-(2-chloro-4-{[5-(2-hydroxyethyl)-5H-pyrrolo[3,2-d]pyrimidin-4-yl]amino}phenoxy)benzonitrile,
3-[2-chloro-4-({5-[2-(2-hydroxyethoxy)ethyl]-5H-pyrrolo[3,2-d]pyrimidin-4-yl}amino)phenoxy]benzonitrile,
3-[2-chloro-4-(6,7-dihydro-9H-pyrimido[4′,5′:4,5]pyrrolo[2,1-c][1,4]oxazin-4-ylamino)phenoxy]benzonitrile hydrochloride, and
(2E)-N-[(2E)-3-(4-{[3-chloro-4-(3-cyanophenoxy)phenyl]amino}-5-methyl-5H-pyrrolo[3,2-d]pyrimidin-6-yl)prop-2-en-1-yl]-4-(dimethylamino)but-2-enamide
are excluded.
[2c] The compound of the above-mentioned [1c], wherein R^{1c} is a hydrogen atom.
[3c] A compound selected from the following:
2-{2-[4-({3-chloro-4-[3-(1,3-thiazol-5-yl)phenoxy]phenyl}amino)-5H-pyrrolo[3,2-d]pyrimidin-5-yl]ethoxy}ethanol,
N-(tert-butyl)-3-[2-chloro-4-({5-[2-(2-hydroxyethoxy)ethyl]-5H-pyrrolo[3,2-d]pyrimidin-4-yl}amino)phenoxy]benzamide,
3-[2-chloro-4-({5-[2-(2-hydroxyethoxy)ethyl]-5H-pyrrolo[3,2-d]pyrimidin-4-yl}amino)phenoxy]-N-(2-hydroxy-1,1-dimethylethyl)benzamide,
N-(tert-butyl)-3-(2-chloro-4-{[5-(2-hydroxyethyl)-5H-pyrrolo[3,2-d]pyrimidin-4-yl]amino}phenoxy)benzamide,
N-(3-{2-chloro-4-[(6-cyano-5-methyl-5H-pyrrolo[3,2-d]pyrimidin-4-yl)amino]phenoxy}phenyl)cyclopropanecarboxamide,
N-(tert-butyl)-5-(2-chloro-4-{[5-(2-hydroxyethyl)-5H-pyrrolo[3,2-d]pyrimidin-4-yl]amino}phenoxy)-2-fluorobenzamide,
N-{2-[4-({3-chloro-4-[3-(dimethylamino)phenoxy]phenyl}amino)-5H-pyrrolo[3,2-d]pyrimidin-5-yl]ethyl}-3-hydroxy-3-methylbutanamide,
N-{2-[4-({3-chloro-4-[3-(dimethylamino)phenoxy]phenyl}amino)-5H-pyrrolo[3,2-d]pyrimidin-5-yl]ethyl}-2-(methylsulfonyl)acetamide,
N-(tert-butyl)-2-[3-(2-chloro-4-{[5-(2-hydroxyethyl)-5H-pyrrolo[3,2-d]pyrimidin-4-yl]amino}phenoxy)phenyl]acetamide,
N-{2-[4-({3-chloro-4-[3-(cyclopropylmethoxy)phenoxy]phenyl}amino)-5H-pyrrolo[3,2-d]pyrimidin-5-yl]ethyl}-2-(methylsulfonyl)acetamide,
N-{2-[4-({3-chloro-4-[3-(2,2-dimethylpropoxy)phenoxy]phenyl}amino)-5H-pyrrolo[3,2-d]pyrimidin-5-yl]ethyl}-2-(methylsulfonyl)acetamide,
2-(methylsulfonyl)-N-{2-[4-({3-methyl-4-[3-(2,2,2-trifluoroethoxy)phenoxy]phenyl}amino)-5H-pyrrolo[3,2-d]pyrimidin-5-yl]ethyl}acetamide,
2-[4-({3-chloro-4-[3-(isopropylsulfonyl)phenoxy]phenyl}amino)-5H-pyrrolo[3,2-d]pyrimidin-5-yl]ethanol, and
N-[2-(4-{[3-chloro-4-(3-cyanophenoxy)phenyl]amino}-5H-pyrrolo[3,2-d]pyrimidin-5-yl)ethyl]-2-(methylsulfonyl)acetamide,
or a salt thereof.
[4c] A prodrug of the compound of the above-mentioned [1c].
[5c] A production method of the compound of the above-mentioned [1c] or a salt thereof, which comprises reacting a compound represented by the formula:

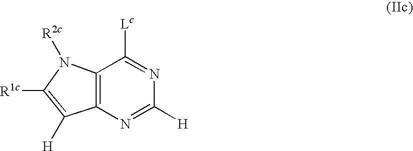

wherein L^{c} is a leaving group, and the other symbols are as defined above,
or a salt thereof and a compound represented by the formula:

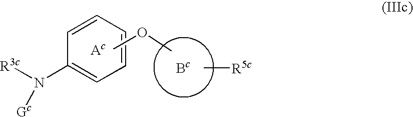

wherein G^{c} is a hydrogen atom or a metal atom, and the other symbols are as defined above,
or a salt thereof.
[6c] A pharmaceutical agent comprising the compound of the above-mentioned [1c] or a salt thereof, or a prodrug thereof.
[7c] The pharmaceutical agent of the above-mentioned [6c], which is a tyrosine kinase inhibitor.
[8c] The pharmaceutical agent of the above-mentioned [6c], which is an agent for the prophylaxis or treatment of cancer.
[9c] The pharmaceutical agent of the above-mentioned [8c], wherein the cancer is breast cancer, ovarian cancer, colorectal cancer, gastric cancer, esophagus cancer, prostate cancer, lung cancer, pancreatic cancer or kidney cancer.
[10c] A method for the prophylaxis or treatment of cancer in a mammal, which comprises administering an effective amount of the compound of the above-mentioned [1c] or a salt thereof, or a prodrug thereof, to the mammal.
[11c] Use of the compound of the above-mentioned [1c] or a salt thereof, or a prodrug thereof, for the production of an agent for the prophylaxis or treatment of cancer.
[12c] The compound of the above-mentioned [1c], which is a compound represented by the formula:

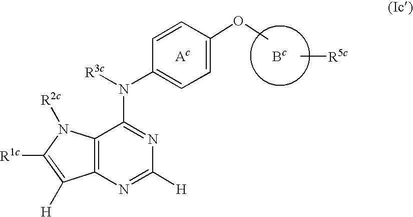

wherein each symbol is as defined above.
[13c] The compound of the above-mentioned [1c], which is a compound represented by the formula:

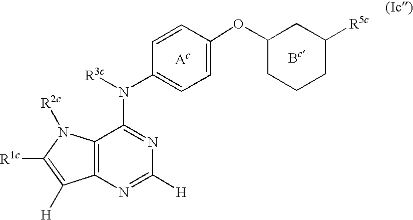

wherein ring B^{c′} is a phenyl group or a cyclohexyl group, each of which is optionally further substituted besides R^{5c}, and the other symbols are as defined above.
[14c] The compound of the above-mentioned [1c], wherein R^{2c} is a C₁₋₆ alkyl group optionally substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl.
[15c] The compound of the above-mentioned [1c], wherein R^{1c} is a hydrogen atom or a cyano group,
R^{2c} is a C₁₋₆ alkyl group optionally substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group,
ring A^{c} is a benzene ring optionally substituted by substituent(s) selected from the group consisting of halogen and methyl,
R^{5c} is
(i) an amino group,
(ii) a mono-C₁₋₆ alkyl-amino group,
(iii) a di-C₁₋₆ alkyl-amino group,
(iv) an optionally halogenated C₁₋₆ alkanoyl-amino group,
(v) a hydroxy-C₁₋₆ alkanoyl-amino group,
(vi) a C₁₋₆ alkanoyl-amino group having hydroxy and halogen,
(vii) a C₃₋₇ cycloalkyl-C₁₋₆ alkanoyl-amino group,
(viii) a C₁₋₆ alkanoyl-amino group having C₃₋₇ cycloalkyl and halogen,
(ix) a C₁₋₆ alkylsulfonyl-C₁₋₆ alkanoyl-amino group,
(x) a C₃₋₇ cycloalkyl-carbonyl-amino group,
(xi) a C₁₋₆ alkoxy-carbonyl-amino group,
(xii) a carbamoyl group,
(xiii) an optionally halogenated C₁₋₆ alkyl-carbamoyl group,
(xiv) a hydroxy-C₁₋₆ alkyl-carbamoyl group,
(xv) a C₁₋₆ alkoxy-C₁₋₆ alkyl-carbamoyl group,
(xvi) a C₆₋₁₄ aryl-C₁₋₆ alkyl-carbamoyl group,
(xvii) a C₂₋₆ alkynyl-carbamoyl group,
(xviii) a piperidyl-C₁₋₆ alkyl-carbamoyl group,
(xix) a morpholinyl-C₁₋₆ alkyl-carbamoyl group,
(xx) a C₃₋₇ cycloalkyl-carbamoyl group optionally substituted by C₁₋₆ alkyl or C₂₋₆ alkynyl,
(xxi) a 5 or 6-membered cyclic amino-carbonyl group optionally containing an oxygen atom,
(xxii) a ureido group,
(xxiii) a C₁₋₆ alkyl-ureido group,
(xxiv) a C₃₋₇ cycloalkyl-ureido group,
(xxv) a 5- to 8-membered heterocyclyl-ureido group containing, besides carbon atoms, 1 to 3 hetero atoms selected from the group consisting of a nitrogen atom, an oxygen atom and a sulfur atom,
(xxvi) a sulfamoyl group optionally substituted by C₁₋₆ alkyl,
(xxvii) a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl,
(xxviii) a C₂₋₆ alkoxy group optionally substituted by substituent(s) selected from the group consisting of C₃₋₇ cycloalkyl, halogen, C₁₋₆ alkoxy and C₁₋₆ alkyl-carbamoyl,
(xxix) a carbamoylmethyl group optionally substituted by C₁₋₆ alkyl,
(xxx) an aminomethyl group optionally substituted by C₁₋₆ alkyl-carbonyl,
(xxxi) a C₁₋₆ alkylsulfonyl group optionally having C₃₋₇ cycloalkyl or halogen, or
(xxxii) a cyano group, and
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen.
[16c] The compound of the above-mentioned [1c] or [12c], wherein
R^{5c} is an amino group optionally substituted by substituent(s) selected from the group consisting of
(i) C₁₋₆ alkyl,
(ii) optionally halogenated C₁₋₆ alkanoyl,
(iii) hydroxy-C₁₋₆ alkanoyl,
(iv) C₁₋₆ alkanoyl having hydroxy and halogen,
(v) C₃₋₇ cycloalkyl-C₁₋₆ alkanoyl,
(vi) C₁₋₆ alkanoyl having C₃₋₇ cycloalkyl and halogen,
(vii) C₁₋₆ alkyl sulfonyl-C₁₋₆ alkanoyl,
(viii) C₃₋₇ cycloalkyl-carbonyl, and
(ix) C₁₋₆ alkoxy-carbonyl,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and
ring A^{c} is a benzene ring optionally substituted by substituent(s) selected from the group consisting of halogen and methyl.
[17c] The compound of the above-mentioned [1c] or [12c], wherein
R^{5c} is a carbamoyl group optionally substituted by substituent(s) selected from the group consisting of
(i) optionally halogenated C₁₋₆ alkyl,
(ii) hydroxy-C₁₋₆ alkyl,
(iii) C₁₋₆ alkoxy-C₁₋₆ alkyl,
(iv) C₆₋₁₄ aryl-C₁₋₆ alkyl,
(v) C₂₋₆ alkynyl,
(vi) piperidyl-C₁₋₆ alkyl,
(vii) morpholinyl-C₁₋₆ alkyl, and
(viii) C₃₋₇ cycloalkyl optionally substituted by C₁₋₆ alkyl or C₂₋₆ alkynyl,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and
ring A^{c} is a benzene ring optionally substituted by substituent(s) selected from the group consisting of halogen and methyl.
[18c] The compound of the above-mentioned [1c] or [12c], wherein
R⁵ is a ureido group optionally substituted by substituent(s) selected from the group consisting of
(i) C₁₋₆ alkyl,
(ii) C₃₋₇ cycloalkyl, and
(iii) 5- to 8-membered heterocyclic group containing, besides carbon atoms, 1 to 3 hetero atoms selected from the group consisting of a nitrogen atom, an oxygen atom and a sulfur atom,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and
ring A^{c} is a benzene ring optionally substituted by substituent(s) selected from the group consisting of halogen and methyl.
[19c] The compound of the above-mentioned [1c] or [12c], wherein
R^{5c} is a sulfamoyl group optionally substituted by C₁₋₆ alkyl,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and
ring A^{c} is a benzene ring optionally substituted by substituent(s) selected from the group consisting of halogen and methyl.
[20c] The compound of the above-mentioned, [1c] or [12c], wherein
R^{5c} is a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and
ring A^{c} is a benzene ring optionally substituted by substituent(s) selected from the group consisting of halogen and methyl.
[21c] The compound of any one of the above-mentioned [16c] to [20c], wherein
R^{2c} is a C₁₋₆ alkyl group substituted by substituent(s), selected from the group consisting of
(i) —NH—CO—CR^{7c}R^{8c}—SO₂—C₁₋₄ alkyl
wherein R^{7c} and R^{8c} are the same or different and each is a hydrogen atom or a C₁₋₄ alkyl group,
(ii) —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino.
[22c] The compound of the above-mentioned [1c] or [12c], wherein
R^{2c} is a C₁₋₆ alkyl group substituted by a group represented by —NR^{6ca}—CO—(CH₂)ₙ₁—SO₂-optionally halogenated C₁₋₄ alkyl
wherein n1 is an integer of 1 to 4, R^{6ca} is a hydrogen atom or a C₁₋₄ alkyl group, and —(CH₂)ₙ₁— is optionally substituted by C₁₋₄ alkyl,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group optionally (a) mono-substituted by C₁₋₆ alkanoyl optionally having C₁₋₆ alkylsulfonyl, or (b) mono- or di-substituted by C₁₋₆ alkyl,
(ii) a carbamoyl group optionally substituted by C₁₋₆ alkyl,
(iii) a 5- to 8-membered heterocyclic group containing, besides carbon atoms, 1 to 3 hetero atoms selected from the group consisting of a nitrogen atom, an oxygen atom sand a sulfur atom, which is optionally substituted by optionally halogenated C₁₋₆ alkyl,
(iv) a C₂₋₆ alkoxy group optionally substituted by C₃₋₇ cycloalkyl, halogen, C₁₋₆ alkoxy or C₁₋₆ alkyl-carbamoyl,
(v) an aminomethyl group optionally substituted by C₁₋₆ alkyl-carbonyl,
(vi) a C₁₋₆ alkylsulfonyl group optionally substituted by C₃₋₇ cycloalkyl, or
(vii) a cyano group, and
ring B^{c} is a C₆₋₁₄ aryl group optionally further substituted, besides R^{5c}, by substituent(s) selected from the group consisting of optionally halogenated C₁₋₆ alkyl and halogen.
[23c] The compound of the above-mentioned [22c], wherein
R^{2c} is a C₁₋₆ alkyl group substituted by a group represented by —NH—CO—CR^{7c}R^{8c}—SO₂—C₁₋₄ alkyl
wherein R^{7c} and R^{8c} are the same or different and each is a hydrogen atom or a C₁₋₄ alkyl group.
[24c] The compound of the above-mentioned [1c] or [12c], wherein
R^{2c} is a C₁₋₆ alkyl group substituted by a group represented by —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group optionally (a) mono-substituted by C₁₋₆ alkanoyl optionally having hydroxy, or (b) mono- or di-substituted by C₁₋₆ alkyl,
(ii) a carbamoyl group optionally substituted by C₁₋₆ alkyl,
(iii) a 5- to 8-membered heterocyclic group containing, besides carbon atoms, 1 to 3 hetero atoms selected from the group consisting of a nitrogen atom, an oxygen atom and a sulfur atom, which is optionally substituted by optionally halogenated C₁₋₆ alkyl,
(iv) a C₂₋₆ alkoxy group optionally substituted by C₃₋₇ cycloalkyl, halogen, C₁₋₆ alkoxy or C₁₋₆ alkyl-carbamoyl,
(v) an aminomethyl group optionally substituted by C₁₋₆ alkyl-carbonyl,
(vi) a C₁₋₆ alkylsulfonyl group optionally substituted by C₃₋₇ cycloalkyl, or
(vii) a cyano group, and
ring B^{c} is a C₆₋₁₄ aryl group optionally further substituted, besides R^{5c}, by substituent(s) selected from the group consisting of optionally halogenated C₁₋₆ alkyl and halogen.
[25c] The compound of the above-mentioned [24c], wherein
R^{2c} is a C₁₋₆ alkyl group substituted by a group represented by —NH—CO—CH₂—CR^{9c}R^{10c}—OH
wherein R^{9c} and R^{10c} are the same or different and each is a C₁₋₄ alkyl group.
[26c] The compound of the above-mentioned [1c] or [12c], wherein
R^{2c} is a C₁₋₆ alkyl group substituted by a group represented by —O—(CH₂)ₙ₃—OH
wherein n3 is an integer of 1 to 4, and —(CH₂)ₙ₃— is optionally substituted by C₁₋₄ alkyl,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group,
(ii) a C₁₋₆ alkyl-amino group,
(iii) an optionally halogenated C₁₋₆ alkanoyl-amino group,
(iv) a hydroxy-C₁₋₆ alkanoyl-amino group,
(v) a C₁₋₆ alkanoyl-amino group having hydroxy and halogen,
(vi) a C₃₋₇ cycloalkyl-C₁₋₆ alkanoyl-amino group,
(vii) a C₁₋₆ alkanoyl-amino group having C₃₋₇ cycloalkyl and halogen,
(viii) a C₃₋₇ cycloalkyl-carbonyl-amino group,
(ix) a C₁₋₆ alkoxy-carbonyl-amino group,
(x) a carbamoyl group,
(xi) an optionally halogenated C₁₋₆ alkyl-carbamoyl group,
(xii) a hydroxy-C₁₋₆ alkyl-carbamoyl group,
(xiii) a C₁₋₆ alkoxy-C₁₋₆ alkyl-carbamoyl group,
(xiv) a C₃₋₇ cycloalkyl-carbamoyl group,
(xv) a ureido group,
(xvi) a C₁₋₆ alkyl-ureido group,
(xvii) a C₃₋₇ cycloalkyl-ureido group,
(xviii) a 5- to 8-membered heterocyclyl-ureido group containing, besides carbon atoms, 1 to 3 hetero atoms selected from the group consisting of a nitrogen atom, an oxygen atom and a sulfur atom,
(xix) a 5 or 6-membered cyclic amino-carbonyl group optionally containing an oxygen atom,
(xx) a 5- to 8-membered heterocyclic group containing, besides carbon atoms, 1 to 3 hetero atoms selected from the group consisting of a nitrogen atom, an oxygen atom and a sulfur atom, which is optionally substituted by optionally halogenated C₁₋₆ alkyl or C₁₋₆ alkoxy-carbonyl,
(xxi) an optionally halogenated C₂₋₆ alkoxy group,
(xxii) a C₁₋₆ alkylsulfonyl group, or
(xxiii) a cyano group, and
ring B^{c} is a C₆₋₁₄ aryl group optionally further substituted, besides R^{5c}, by substituent(s) selected from the group consisting of optionally halogenated C₁₋₆ alkyl and halogen.
[27c] The compound of the above-mentioned [1c] or [12c], wherein
R^{2c} is a C₁₋₆ alkyl group substituted by hydroxy,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group optionally (a) mono-substituted by C₁₋₆ alkanoyl optionally having hydroxy, or (b) mono- or di-substituted by C₁₋₆ alkyl,
(ii) a carbamoyl group optionally substituted by optionally halogenated C₁₋₆ alkyl,
(iii) a C₃₋₇ cycloalkyl-carbamoyl group optionally substituted by C₁₋₆ alkyl or C₂₋₆ alkynyl,
(iv) a C₆₋₁₄ aryl-C₁₋₆ alkyl-carbamoyl group,
(v) a hydroxy-C₁₋₆ alkyl-carbamoyl group,
(vi) a morpholinyl-C₁₋₆ alkyl-carbamoyl group,
(vii) a C₂₋₆ alkynyl-carbamoyl group,
(viii) a carbamoylmethyl group optionally substituted by C₁₋₆ alkyl,
(ix) a C₂₋₆ alkoxy group optionally substituted by C₃₋₇ cycloalkyl, halogen, C₁₋₆ alkoxy or C₁₋₆ alkyl-carbamoyl,
(x) an aminomethyl group optionally substituted by C₁₋₆ alkoxy-carbonyl, or
(xi) a C₁₋₆ alkylsulfonyl group optionally substituted by C₃₋₇ cycloalkyl, and
ring B^{c} is a C₆₋₁₄ aryl group optionally further substituted, besides R^{5c}, by substituent(s) selected from the group consisting of optionally halogenated C₁₋₆ alkyl and halogen.
[28c] The compound of the above-mentioned [1c] or [12c], wherein
R^{1c} is a cyano group or an optionally halogenated C₁₋₆ alkyl group,
R^{2c} is
(i) a C₁₋₆ alkyl group, or
(ii) a C₁₋₆ alkyl group substituted by substituent(s) selected from the group consisting of
(a) —NR^{6c}—CO—(CH₂)ₙ—SO₂-optionally halogenated C₁₋₄ alkyl,
(b) —NR^{6c}—CO—(CH₂)ₙ—OH,
(c) —O—(CH₂)ₙ—OH, and
(d) hydroxy
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group,
ring A^{c} is a benzene ring optionally substituted by substituent(s) selected from the group consisting of halogen and methyl,
R^{5c} is
(i) an amino group,
(ii) a C₁₋₆ alkyl-amino group,
(iii) an optionally halogenated C₁₋₆ alkanoyl-amino group,
(iv) a hydroxy-C₁₋₆ alkanoyl-amino group,
(v) a C₁₋₆ alkanoyl-amino group having hydroxy and halogen,
(vi) a C₃₋₇ cycloalkyl-C₁₋₆ alkanoyl-amino group,
(vii) a C₁₋₆ alkanoyl-amino group having C₃₋₇ cycloalkyl and halogen,
(viii) a C₁₋₆ alkylsulfonyl-C₁₋₆ alkanoyl-amino group,
(ix) a C₃₋₇ cycloalkyl-carbonyl-amino group,
(x) a C₁₋₆ alkoxy-carbonyl-amino group,
(xi) a carbamoyl group,
(xii) an optionally halogenated C₁₋₆ alkyl-carbamoyl group,
(xiii) a hydroxy-C₁₋₆ alkyl-carbamoyl group,
(xiv) a C₁₋₆ alkoxy-C₁₋₆ alkyl-carbamoyl group,
(xv) a C₃₋₇ cycloalkyl-carbamoyl group,
(xvi) a 5 or 6-membered cyclic amino-carbonyl group optionally containing an oxygen atom,
(xvii) a ureido group,
(xviii) a C₁₋₆ alkyl-ureido group,
(xix) a C₃₋₇ cycloalkyl-ureido group,
(xx) a 5- to 8-membered heterocyclyl-ureido group containing, besides carbon atoms, 1 to 3 hetero atoms selected from the group consisting of a nitrogen atom, an oxygen atom and a sulfur atom,
(xxi) a sulfamoyl group optionally substituted by C₁₋₆ alkyl, or
(xxii) a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl, and
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen.
[29c] The compound of any one of the above-mentioned [14c] to [20c] and [28c], wherein ring B^{c} is a phenyl group or a cyclohexyl group, each of which is optionally further substituted, besides R^{5c}, by substituent(s) selected from the group consisting of optionally halogenated C₁₋₆ alkyl and halogen, and is substituted by R^{5c} at the meta-position of the phenyl group or the β-position of the cyclohexyl group.
[30c] The compound of any one of the above-mentioned [22c] to [27c], wherein ring B^{c} is a phenyl group optionally further substituted, besides R^{5c}, by substituent(s) selected from the group consisting of optionally halogenated C₁₋₆ alkyl and halogen, which phenyl is substituted by R^{5c} at the meta-position of the phenyl group.
[1d] A compound represented by the formula:

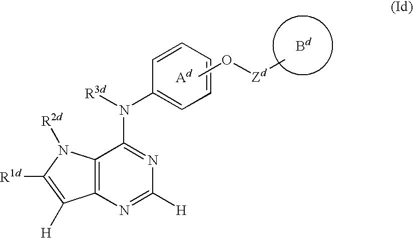

wherein
R^{1d} is a hydrogen atom, or an optionally substituted group bonded via a carbon atom, a nitrogen atom or an oxygen atom,
R^{2d} is an optionally substituted group bonded via a carbon atom or a sulfur atom, or,
R^{1d} and R^{2d}, or R^{2d} and R^{3d} are optionally bonded to form an optionally substituted ring structure,
R^{3d} is a hydrogen atom or an optionally substituted aliphatic hydrocarbon group, or R^{3d} is optionally bonded to the carbon atom on the adjacent benzene ring to form an optionally substituted ring structure,
ring A^{d} is an optionally substituted benzene ring,
Z^{d} is an optionally substituted C₁₋₃ alkylene,
ring B^{d} is an optionally substituted heterocyclic group,
or a salt thereof,
provided that
ethyl 5-[(4-{[3-chloro-4-(pyridin-2-ylmethoxy)phenyl]amino}-5H-pyrrolo[3,2-d]pyrimidin-5-yl)methyl]-2-furoate,
5-[(4-{[3-chloro-4-(pyridin-2-ylmethoxy)phenyl]amino}-5H-pyrrolo[3,2-d]'pyrimidin-5-yl)methyl]-2-furancarboxylic acid,
2-[2-(4-{[3-chloro-4-(pyridin-2-ylmethoxy)phenyl]amino}-5H-pyrrolo[3,2-d]pyrimidin-5-yl)ethoxy]ethanol, and
N-[2-(4-{[3-chloro-4-(pyridin-2-ylmethoxy)phenyl]amino}-5H-pyrrolo[3,2-d]pyrimidin-5-yl)ethyl]-2-(methylsulfonyl)acetamide
are excluded.
[2d] The compound of the above-mentioned [1d], which is a compound represented by the formula:

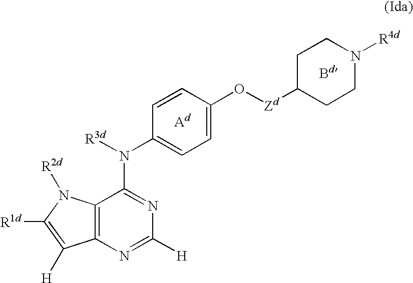

wherein R^{4d} is an acyl group or an optionally substituted ureido group, ring B^{d′} is a piperidyl group optionally further substituted besides R^{4d}, and the other symbols are as defined above.
[3d] A compound selected from the following:
tert-butyl 4-{[2-chloro-4-({5-[2-(2-hydroxyethoxy)ethyl]-5H-pyrrolo[3,2-d]pyrimidin-4-yl}amino)phenoxy]methyl}piperidine-1-carboxylate, and
tert-butyl 4-[(2-chloro-4-{[5-(2-hydroxyethyl)-5H-pyrrolo[3,2-d]pyrimidin-4-yl]amino}phenoxy)methyl]piperidine-1-carboxylate,
or a salt thereof.
[4d] A prodrug of the compound of the above-mentioned [1d].
[5d] A production method of the compound of the above-mentioned [1d] or a salt thereof, which comprises reacting a compound represented by the formula:

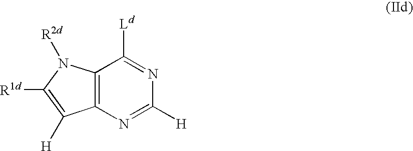

wherein L^{d} is a leaving group, and the other symbols are as defined above,
or a salt thereof and a compound represented by the formula:

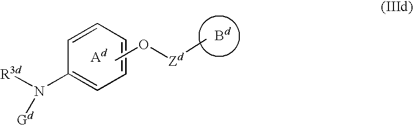

wherein G^{d} is a hydrogen atom or a metal atom, and the other symbols are as defined above,
or a salt thereof.
[6d] A pharmaceutical agent comprising the compound of the above-mentioned [1d] or a salt thereof, or a prodrug thereof.
[7d] The pharmaceutical agent of the above-mentioned [6d], which is a tyrosine kinase inhibitor.
[8d] The pharmaceutical agent of the above-mentioned [6d], which is an agent for the prophylaxis or treatment of cancer.
[9d] The pharmaceutical agent of the above-mentioned [8d], wherein the cancer is breast cancer, ovarian cancer, colorectal cancer, gastric cancer, esophagus cancer, prostate cancer, lung cancer, pancreatic cancer or kidney cancer.
[10d] A method for the prophylaxis or treatment of cancer in a mammal, which comprises administering an effective amount of the compound of the above-mentioned [1d] or a salt thereof, or a prodrug thereof, to the mammal.
[11d] Use of the compound of the above-mentioned [1d] or a salt thereof, or a prodrug thereof, for the production of an agent for the prophylaxis or treatment of cancer.
[12d] The compound of the above-mentioned [1d], which is a compound represented by the formula:

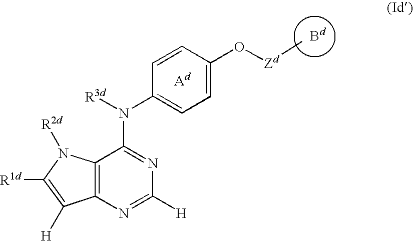

wherein each symbol is as defined above.
[13d] The compound of the above-mentioned [2d], wherein R^{1d} is a hydrogen atom, a cyano group or an optionally halogenated C₁₋₆ alkyl group,
R^{2d} is
(i) a C₁₋₆ alkyl group, or
(ii) a C₁₋₆ alkyl group substituted by substituent(s) selected from the group consisting of
(a) —NR^{6d}—CO—(CH₂)ₙ—SO₂-optionally halogenated C₁₋₄ alkyl,
(b) —NR^{6d}—CO—(CH₂)ₙ—OH,
(c) —O—(CH₂)ₙ—OH, and
(d) hydroxy
wherein n is an integer of 1 to 4, R^{6d} is a hydrogen atom or a C₁₋₄ alkyl group, and —(CH₂)ₙ— is optionally substituted by C₁₋₄ alkyl,
R^{3d} is a hydrogen atom or a C₁₋₆ alkyl group,
ring A^{d} is a benzene ring optionally substituted by substituent(s) selected from the group consisting of halogen and methyl,
Z^{d} is methylene,
ring B^{d′} is a piperidyl group, and
R^{4d} is a C₁₋₆ alkoxy-carbonyl group, a C₅₋₈ cycloalkyl-carbonyl group, a C₁₋₆ alkyl-ureido group or a C₅₋₈ cycloalkyl-ureido group.
[14d] The compound of the above-mentioned [2d], wherein
R^{3d} is a hydrogen atom, and
ring A^{d} is a benzene ring optionally substituted by substituent(s) selected from the group consisting of halogen and methyl.
[1e] A compound represented by the formula:

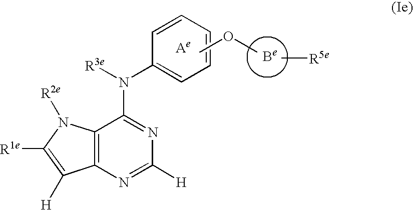

wherein
R^{1e} is a hydrogen atom, or an optionally substituted group bonded via a carbon atom, a nitrogen atom or an oxygen atom,
R^{2e} is an optionally substituted group bonded via a carbon atom or a sulfur atom, or,
R^{1e} and R^{2e}, or R^{2e} and R^{3e} are optionally bonded to form an optionally substituted ring structure,
R^{3e} is a hydrogen atom or an optionally substituted aliphatic hydrocarbon group, or R^{3e} is optionally bonded to the carbon atom on the adjacent benzene ring to form an optionally substituted ring structure,
ring A^{e} is an optionally substituted benzene ring,
R^{5e} is
(i) a linear alkyl group substituted by optionally substituted heterocyclic group,
(ii) a linear alkyl group substituted by optionally substituted imino,
(iii) a linear alkyl group substituted by optionally substituted aryl, which is optionally further halogenated or hydroxylated,
(iv) an optionally substituted branched alkyl group,
(v) an optionally substituted alkenyl group,
(vi) a hydroxy group substituted by optionally substituted aryl,
(vii) a hydroxy group substituted by C₁₋₆ alkyl,
(viii) a hydroxy group substituted by halogenated C₂₋₆ alkyl,
(ix) a halogenated C₂₋₆ alkyl group,
(x) an optionally substituted cycloalkyl group, or
(xi) a C₁₋₆ alkyl-carbonyl group optionally substituted by optionally substituted aryl, and
ring B^{e} is a C₆₋₁₄ aryl group optionally further substituted besides R^{5e},
or a salt thereof,
provided that
2-(2-{4-[(3-chloro-4-{4-[3-(1H-imidazol-1-yl)propyl]phenoxy}phenyl)amino]-5H-pyrrolo[3,2-d]pyrimidin-5-yl}ethoxy)ethanol dihydrochloride,
2-(2-{4-[(3-chloro-4-{4-[4-(1H-1,2,3-triazol-1-yl)butyl]phenoxy}phenyl)amino]-5H-pyrrolo[3,2-d]pyrimidin-5-yl}ethoxy)ethanol, and
1-{3-[2-chloro-4-({5-[2-(2-hydroxyethoxy)ethyl]-5H-pyrrolo[3,2-d]pyrimidin-4-yl}amino)phenoxy]phenyl}ethanone
are excluded.
[2e] The compound of the above-mentioned [1e], wherein the “linear alkyl group substituted by optionally substituted heterocyclic group” for R^{5e} is
(i) a methyl group substituted by optionally substituted heterocyclic group, or
(ii) a linear alkyl group substituted by substituted heterocyclic group.
[3e] A compound selected from the following:
2-[4-({3-chloro-4-[3-(1,1-difluoroethyl)phenoxy]phenyl}amino)-5H-pyrrolo[3,2-d]pyrimidin-5-yl]ethanol,
(1Z)-1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one O-ethyloxime,
1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-ol,
1-[3-(2-chloro-4-{[5-(2-hydroxyethyl)-5H-pyrrolo[3,2-d]pyrimidin-4-yl]amino}phenoxy)phenyl]-3,3-dimethylbutan-1-one,
N-(2-{4-[(3-methyl-4-{3-[(1E)-3-methylbut-1-en-1-yl]phenoxy}phenyl)amino]-5H-pyrrolo[3,2-d]pyrimidin-5-yl}ethyl)-2-(methylsulfonyl)acetamide, and
N-{2-[4-({3-chloro-4-[3-(1-cyanocyclopropyl)phenoxy]phenyl}amino)-5H-pyrrolo[3,2-d]pyrimidin-5-yl]ethyl}-2-(methylsulfonyl)acetamide,
or a salt thereof.
[4e] A prodrug of the compound of the above-mentioned [1e].
[5e] A production method of the compound of the above-mentioned [1e] or a salt thereof, which comprises reacting a compound represented by the formula:

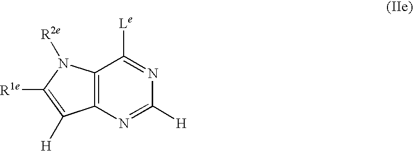

wherein L^{e} is a leaving group, and the other symbols are as defined above,
or a salt thereof and a compound represented by the formula:

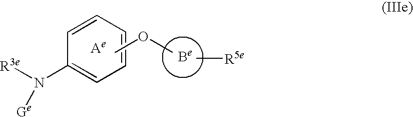

wherein G^{e} is a hydrogen atom or a metal atom, and the other symbols are as defined above,
or a salt thereof.
[6e] A pharmaceutical agent comprising the compound of the above-mentioned [1e] or a salt thereof, or a prodrug thereof.
[7e] The pharmaceutical agent of the above-mentioned [6e], which is a tyrosine kinase inhibitor.
[8e] The pharmaceutical agent of the above-mentioned [6e], which is an agent for the prophylaxis or treatment of cancer.
[9e] The pharmaceutical agent of the above-mentioned [8e], wherein the cancer is breast cancer, ovarian cancer, colorectal cancer, gastric cancer, esophagus cancer, prostate cancer, lung cancer, pancreatic cancer or kidney cancer.
[10e] A method for the prophylaxis or treatment of cancer in a mammal, which comprises administering an effective amount of the compound of the above-mentioned [1e] or a salt thereof, or a prodrug thereof, to the mammal.
[11e] Use of the compound of the above-mentioned [1e] or a salt thereof, or a prodrug thereof, for the production of an agent for the prophylaxis or treatment of cancer.
[12e] The compound of the above-mentioned [1e], which is a compound represented by the formula:

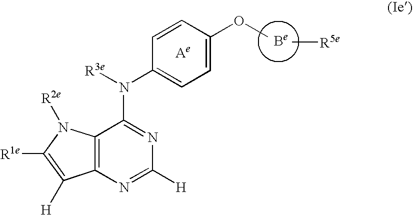

wherein each symbol is as defined above.
[13e] The compound of the above-mentioned [1e], wherein
R^{1e} is a hydrogen atom, a cyano group or an optionally halogenated C₁₋₆ alkyl group,
R^{2e} is
(i) a C₁₋₆ alkyl group, or
(ii) a C₁₋₆ alkyl group substituted by substituent(s) selected from the group consisting of
(a) —NR^{6e}—CO—(CH₂)ₙ—SO₂-optionally halogenated C₁₋₄ alkyl,
(b) —NR^{6e}—CO—(CH₂)ₙ—OH,
(c) —O—(CH₂)ₙ—OH, and
(d) hydroxy
wherein n is an integer of 1 to 4, R^{6e} is a hydrogen atom or a C₁₋₄ alkyl group, and —(CH₂)ₙ— is optionally substituted by C₁₋₄ alkyl,
R^{3e} is a hydrogen atom,
ring A^{e} is a benzene ring optionally substituted by substituent(s) selected from the group consisting of halogen and methyl,
R^{5e} is
(i) a 5- to 8-membered heterocyclyl-linear C₁₋₆ alkyl group containing, besides carbon atoms, 1 to 3 hetero atoms selected from the group consisting of a nitrogen atom, an oxygen atom and a sulfur atom, and optionally having C₁₋₆ alkyl,
(ii) a linear C₁₋₆ alkyl group substituted by hydroxyimino or C₁₋₆ alkoxyimino,
(iii) a linear C₁₋₆ alkyl group substituted by C₆₋₁₄ aryl, which is optionally further halogenated or hydroxylated,
(iv) an optionally halogenated branched C₃₋₆ alkyl group,
(v) a C₂₋₆ alkenyl group,
(vi) a hydroxy group substituted by C₆₋₁₄ aryl,
(vii) a hydroxy group substituted by C₁₋₆ alkyl,
(viii) a hydroxy group substituted by halogenated C₂₋₆ alkyl,
(ix) a halogenated C₂₋₆ alkyl group,
(x) a C₃₋₇ cycloalkyl group optionally substituted by cyano or carbamoyl, or
(xi) a C₁₋₆ alkyl-carbonyl group optionally substituted by phenyl, and
ring B^{e} is a C₆₋₁₄ aryl group optionally further substituted, besides R^{5e}, by substituent(s) selected from the group consisting of optionally halogenated C₁₋₆ alkyl and halogen.
[14e] The compound of the above-mentioned [13e], wherein the “5- to 8-membered heterocyclyl-linear C₁₋₆ alkyl group containing, besides carbon atoms, 1 to 3 hetero atoms selected from the group consisting of a nitrogen atom, an oxygen atom and a sulfur atom, and optionally having C₁₋₆ alkyl” for R^{5e} is
(i) a 5- to 8-membered heterocyclyl-methyl group containing, besides carbon atoms, 1 to 3 hetero atoms selected from the group consisting of a nitrogen atom, an oxygen atom and a sulfur atom, and optionally having C₁₋₆ alkyl, or
(ii) a 5- to 8-membered heterocyclyl-linear C₁₋₆ alkyl group containing, besides carbon atoms, 1 to 3 hetero atoms selected from the group consisting of a nitrogen atom, an oxygen atom and a sulfur atom, and having C₁₋₆ alkyl.
[1f] A compound represented by the formula:

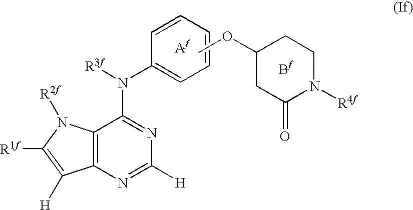

wherein
R^{1f} is a hydrogen atom, or an optionally substituted group bonded via a carbon atom, a nitrogen atom or an oxygen atom,
R^{2f} is an optionally substituted group bonded via a carbon atom or a sulfur atom, or,
R^{1f} and R^{2f}, or R^{2f} and R^{3f} are optionally bonded to form an optionally substituted ring structure,
R^{3f} is a hydrogen atom or an optionally substituted aliphatic hydrocarbon group, or R^{3f} is optionally bonded to the carbon atom on the adjacent benzene ring to form an optionally substituted ring structure,
ring A^{f} is an optionally substituted benzene ring,
ring B^{f} is a piperidyl group optionally further substituted besides R^{4f}, and
R^{4f} is (i) an optionally substituted C₁₋₆ alkyl group, or
(ii) an optionally substituted C₅₋₈ cycloalkyl group,
or a salt thereof.
[2f] The compound of the above-mentioned [1f], wherein
R^{1f} is a hydrogen atom, a cyano group or an optionally halogenated C₁₋₆ alkyl group,
R^{2f} is
(i) a C₁₋₆ alkyl group, or
(ii) a C₁₋₆ alkyl group substituted by substituent(s) selected from the group consisting of
(a) —NR^{6f}—CO—(CH₂)ₙ—SO₂-optionally halogenated C₁₋₄ alkyl,
(b) —NR^{6f}—CO—(CH₂)ₙ—OH,
(c) —O—(CH₂)ₙ—OH, and
(d) hydroxy
wherein n is an integer of 1 to 4, R^{6f} is a hydrogen atom or a C₁₋₄ alkyl group, and —(CH₂)ₙ— is optionally substituted by C₁₋₄ alkyl,
R^{3f} is a hydrogen atom or a C₁₋₆ alkyl group,
ring A^{f} is a benzene ring optionally substituted by substituent(s) selected from the group consisting of halogen and methyl,
ring B^{f} is a piperidyl group, and
R^{4f} is (i) an optionally substituted C₁₋₆ alkyl group, or
(ii) an optionally substituted C₅₋₈ cycloalkyl group.
[3f] The compound of the above-mentioned [1f], wherein
R^{3f} is a hydrogen atom, and
ring A^{f} is a benzene ring optionally substituted by substituent(s) selected from the group consisting of halogen and methyl.
[4f] A prodrug of the compound of the above-mentioned [1f].
[5f] A production method of the compound of the above-mentioned [1f] or a salt thereof, which comprises reacting a compound represented by the formula:

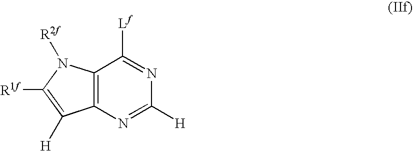

wherein L^{f} is a leaving group, and the other symbols are as defined above,
or a salt thereof and a compound represented by the formula:

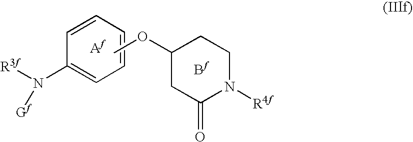

wherein G^{f} is a hydrogen atom or a metal atom, and the other symbols are as defined above,
or a salt thereof.
[6f] A pharmaceutical agent comprising the compound of the above-mentioned [1f] or a salt thereof, or a prodrug thereof.
[7f] The pharmaceutical agent of the above-mentioned [6f], which is a tyrosine kinase inhibitor.
[8f] The pharmaceutical agent of the above-mentioned [6f], which is an agent for the prophylaxis or treatment of cancer.
[9f] The pharmaceutical agent of the above-mentioned [8f], wherein the cancer is breast cancer, ovarian cancer, colorectal cancer, gastric cancer, esophagus cancer, prostate cancer, lung cancer, pancreatic cancer or kidney cancer.
[10f] A method for the prophylaxis or treatment of cancer in a mammal, which comprises administering an effective amount of the compound of the above-mentioned [1f] or a salt thereof, or a prodrug thereof, to the mammal.
[11f] Use of the compound of the above-mentioned [1f] or a salt thereof, or a prodrug thereof, for the production of an agent for the prophylaxis or treatment of cancer.
[12f] The compound of the above-mentioned [1f], which is a compound represented by the formula:

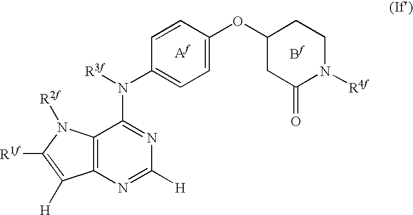

wherein each symbol is as defined above.
[1g] A compound represented by the formula:

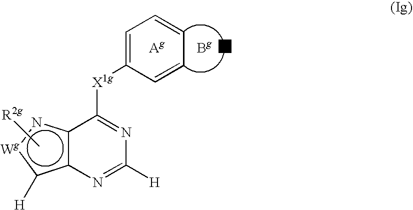

wherein
W^{g} is C(R^{1g}) or N,
ring A^{g} is an optionally substituted benzene ring,
ring B^{g} is an optionally substituted nitrogen-containing heterocycle,
X^{1g} is —NR^{3g}—Y^{1g}—, —O—, —S—, —SO—, —SO₂— or —CHR^{3g}—
wherein R^{3g} is a hydrogen atom or an optionally substituted aliphatic hydrocarbon group, or R^{3g} is optionally bonded to the carbon atom on the benzene ring for ring A^{g} to form an optionally substituted ring structure, and Y^{1g} is a bond, or a C₁₋₄ alkylene or —O—(C₁₋₄ alkylene)-, each of which is optionally substituted, and
R^{1g} is a hydrogen atom, a halogen atom, or an optionally substituted group bonded via a carbon atom, a nitrogen atom or an oxygen atom,
R^{2g} is a hydrogen atom, or an optionally substituted group bonded via a carbon atom or a sulfur atom, or
R^{1g} and R^{2g}, or R^{2g} and R^{3g} are optionally bonded to form an optionally substituted ring structure,
or a salt thereof.
[2g] The compound of the above-mentioned [1g], which is a compound represented by the formula:

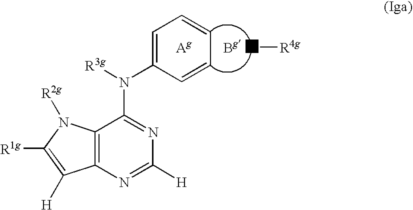

wherein R^{4g} is an optionally substituted hydrocarbon group, ring B^{g′} is a 5 or 6-membered nitrogen-containing heterocycle optionally further substituted besides R^{4g}, and the other symbols are as defined above.
[3g] The compound of the above-mentioned [2g], wherein
R^{1g} is a hydrogen atom, a halogen atom, a cyano group or an optionally halogenated C₁₋₆ alkyl group,
R^{2g} is a hydrogen atom or an optionally substituted C₁₋₆ alkyl group,
R^{3g} is a hydrogen atom or a C₁₋₆ alkyl group,
R^{4g} is (i) an optionally substituted C₆₋₁₄ aryl-C₁₋₈ alkyl group, (ii) an optionally substituted heterocyclyl-C₁₋₈ alkyl group, (iii) a C₁₋₆ alkyl group, or (iv) an optionally substituted C₆₋₁₄ aryl group.
[4g] The compound of the above-mentioned [2g], wherein
R^{1g} is a hydrogen atom, a halogen atom, a cyano group or an optionally halogenated C₁₋₆ alkyl group,
R^{2g} is
(i) a hydrogen atom,
(ii) a C₁₋₆ alkyl group, or
(iii) a C₁₋₆ alkyl group substituted by substituent(s) selected from the group consisting of
(a) —O—(CH₂)ₙ—OH,
(b) —NR^{5g}—CO—(CH₂)ₙ—OH,
(c) —NR^{5g}—CO—(CH₂)ₙ—SO₂-optionally halogenated C₁₋₄ alkyl,
(d) hydroxy, and
(e) amino
wherein n is an integer of 1 to 4, R^{5g} is a hydrogen atom or a C₁₋₄ alkyl group, and —(CH₂)ₙ— is optionally substituted by C₁₋₄ alkyl,
R^{3g} is a hydrogen atom or a C₁₋₆ alkyl group,

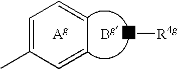

is the formula

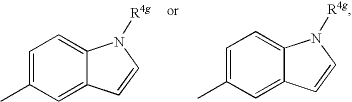

and
R^{4g} is (i) a C₆₋₁₄ aryl-C₁₋₈ alkyl group optionally substituted by substituent(s) selected from the group consisting of halogen, C₁₋₆ alkyl-carbamoyl and halo C₁₋₆ alkoxy, (ii) an optionally substituted heterocyclyl-C₁₋₈ alkyl group, or (iii) an optionally substituted C₆₋₁₄ aryl group.
[5g] A compound selected from the following:
N-[2-(4-{[1-(3-fluorobenzyl)-1H-indazol-5-yl]amino}-5H-pyrrolo[3,2-d]pyrimidin-5-yl)ethyl]-2-(methylsulfonyl)acetamide,
N-[2-(4-{[1-(3-fluorobenzyl)-1H-indol-5-yl]amino}-5H-pyrrolo[3,2-d]pyrimidin-5-yl)ethyl]-3-hydroxy-3-methylbutanamide,
N-(tert-butyl)-3-[(5-{[5-(2-hydroxyethyl)-5H-pyrrolo[3,2-d]pyrimidin-4-yl]amino}-1H-indol-1-yl)methyl]benzamide,
N-(tert-butyl)-3-[(5-{[5-(2-hydroxyethyl)-5H-pyrrolo[3,2-d]pyrimidin-4-yl]amino}-1H-indazol-1-yl)methyl]benzamide, and
N-(tert-butyl)-6-[(5-{[5-(2-hydroxyethyl)-5H-pyrrolo[3,2-d]pyrimidin-4-yl]amino}-1H-indol-1-yl)methyl]pyridine-2-carboxamide,
or a salt thereof.
[6g] A prodrug of the compound of the above-mentioned [1g].
[7g] A production method of the compound of the above-mentioned [1g] or a salt thereof, which comprises reacting a compound represented by the formula:

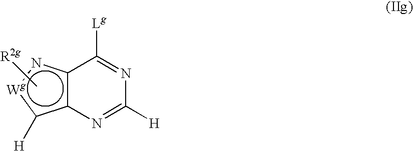

wherein L^{g} is a leaving group, and the other symbols are as defined above,
or a salt thereof and a compound represented by the formula:

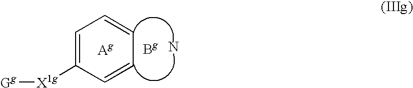

wherein G^{g} is a hydrogen atom or a metal atom, and the other symbols are as defined above,
or a salt thereof.
[8g] A pharmaceutical agent comprising the compound of the above-mentioned [1g] or a salt thereof, or a prodrug thereof.
[9g] The pharmaceutical agent of the above-mentioned [8g], which is a tyrosine kinase inhibitor.
[10g] The pharmaceutical agent of the above-mentioned [8g], which is an agent for the prophylaxis or treatment of cancer.
[11g] The pharmaceutical agent of the above-mentioned [10g], wherein the cancer is breast cancer, ovarian cancer, colorectal cancer, gastric cancer, esophagus cancer, prostate cancer, lung cancer, pancreatic cancer or kidney cancer.
[12g] A method for the prophylaxis or treatment of cancer in a mammal, which comprises administering an effective amount of the compound of the above-mentioned [1g] or a salt thereof, or a prodrug thereof, to the mammal.
[13g] Use of the compound of the above-mentioned [1g] or a salt thereof, or a prodrug thereof, for the production of an agent for the prophylaxis or treatment of cancer.
[1h] A compound represented by the formula:

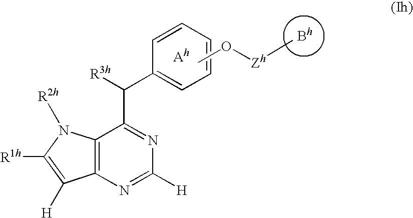

wherein
R^{1h} is a halogen atom or a halogenated C₁₋₆ alkyl group,
R^{2h} is a hydrogen atom, or an optionally substituted group bonded via a carbon atom or a sulfur atom, or R^{1h} and R^{2h}, or R^{2h} and R^{3h} are bonded to form an optionally substituted ring structure,
R^{3h} is a hydrogen atom or an optionally substituted aliphatic hydrocarbon group, or R^{3h} is optionally bonded to the carbon atom on the adjacent benzene ring to form an optionally substituted ring structure,
Z^{h} is a bond or an optionally substituted C₁₋₃ alkylene,
ring A^{h} is an optionally substituted benzene ring, and
ring B^{h} is (i) an optionally substituted C₆₋₁₄ aryl group,
(ii) an optionally substituted heterocyclic group, or
(iii) an optionally substituted C₅₋₈ cycloalkyl group,
or a salt thereof.
[2h] The compound of the above-mentioned [1h], which is a compound represented by the formula:

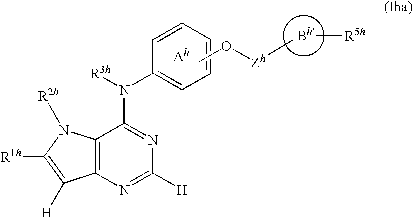

wherein
R^{5h} is
(i) an optionally substituted amino group,
(ii) an optionally substituted carbamoyl group,
(iii) an optionally substituted ureido group,
(iv) an optionally substituted sulfamoyl group,
(v) an optionally substituted heterocyclic group,
(vi) an optionally substituted hydrocarbon group,
(vii) a halogen atom, or
(viii) an optionally substituted, carboxyl group, and
ring B^{h′} is (i) a C₆₋₁₄ aryl group, (ii) a heterocyclic group, or (iii) a C₅₋₈ cycloalkyl group, each of which is optionally further substituted besides R^{5h}, and the other symbols are as defined above.
[3h] A compound selected from the following:
N-(3-{2-chloro-4-[(6-chloro-5-methyl-5H-pyrrolo[3,2-d]pyrimidin-4-yl)amino]phenoxy}phenyl)cyclopropanecarboxamide,
6-chloro-N-{3-chloro-4-[3-(trifluoromethyl)phenoxy]phenyl}-5-methyl-5H-pyrrolo[3,2-d]pyrimidine-4-amine,
N-[3-(2-chloro-4-{[6-chloro-5-(2-hydroxyethyl)-5H-pyrrolo[3,2-d]pyrimidin-4-yl]amino}phenoxy)phenyl]cyclopropanecarboxamide, and
N-(tert-butyl)-3-(2-chloro-4-{[6-chloro-5-(2-hydroxyethyl)-5H-pyrrolo[3,2-d]pyrimidin-4-yl]amino}phenoxy)benzamide,
or a salt thereof.
[4h] A prodrug of the compound of the above-mentioned [1h].
[5h] A production method of the compound of the above-mentioned [1h] or a salt thereof, which comprises reacting a compound represented by the formula:

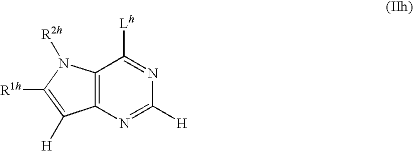

wherein L^{h} is a leaving group, and the other symbols are as defined above,
or a salt thereof and a compound represented by the formula:

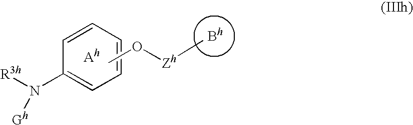

wherein G^{h} is a hydrogen atom or a metal atom, and the other symbols are as defined above,
or a salt thereof.
[6h] A pharmaceutical agent comprising the compound of the above-mentioned [1h] or a salt thereof, or a prodrug thereof.
[7h] The pharmaceutical agent of the above-mentioned [6h], which is a tyrosine kinase inhibitor.
[8h] The pharmaceutical agent of the above-mentioned [6h], which is an agent for the prophylaxis or treatment of cancer.
[9h] The pharmaceutical agent of the above-mentioned [8h], wherein the cancer is breast cancer, ovarian cancer, colorectal cancer, gastric cancer, esophagus cancer, prostate cancer, lung cancer, pancreatic cancer or kidney cancer.
[10h] A method for the prophylaxis or treatment of cancer in a mammal, which comprises administering an effective amount of the compound of the above-mentioned [1h] or a salt thereof, or a prodrug thereof, to the mammal.
[11h] Use of the compound of the above-mentioned [1h] or a salt thereof, or a prodrug thereof, for the production of an agent for the prophylaxis or treatment of cancer.
[12h] The compound of the above-mentioned [1h], which is a compound represented by the formula:

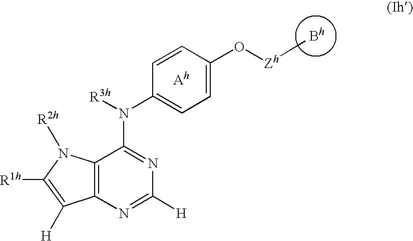

wherein each symbol is as defined above.
[13h] The compound of the above-mentioned [2h], wherein
R^{1h} is a halogen atom or an optionally halogenated C₁₋₆ alkyl group,
R^{2h} is
(i) a hydrogen atom,
(ii) a C₁₋₆ alkyl group, or
(iii) a C₁₋₆ alkyl group substituted by substituent(s) selected from the group consisting of
(a) —O—(CH₂)ₙ—OH,
(b) —NR^{6h}—CO—(CH₂)ₙ—OH,
(c) —NR^{6h}—CO—(CH₂)ₙ—SO₂-optionally halogenated C₁₋₄ alkyl, and
(d) hydroxy
wherein n is an integer of 1 to 4, R^{6h} is a hydrogen atom or a C₁₋₄ alkyl group, and —(CH₂)ₙ— is optionally substituted by C₁₋₄ alkyl,
R^{3h} is a hydrogen atom or a C₁₋₆ alkyl group,
Z^{h} is a bond or methylene,
ring A^{h} is a benzene ring optionally substituted by substituent(s) selected from the group consisting of halogen and methyl,
R^{5h} is
(i) an amino group,
(ii) a C₁₋₆ alkyl-amino group,
(iii) an optionally halogenated C₁₋₆ alkanoyl-amino group,
(iv) a hydroxy-C₁₋₆ alkanoyl-amino group,
(v) a C₁₋₆ alkanoyl-amino group having hydroxy and halogen,
(vi) a C₃₋₇ cycloalkyl-C₁₋₆ alkanoyl-amino group,
(vii) a C₁₋₆ alkanoyl-amino group having C₃₋₇ cycloalkyl and halogen,
(viii) a C₁₋₆ alkylsulfonyl-C₁₋₆ alkanoyl-amino group,
(ix) a C₃₋₇ cycloalkyl-carbonyl-amino group,
(x) a C₁₋₆ alkoxy-carbonyl-amino group,
(xi) a carbamoyl group,
(xii) an optionally halogenated C₁₋₆ alkyl-carbamoyl group,
(xiii) a hydroxy-C₁₋₆ alkyl-carbamoyl group,
(xiv) a C₁₋₆ alkoxy-C₁₋₆ alkyl-carbamoyl group,
(xv) a C₃₋₇ cycloalkyl-carbamoyl group,
(xvi) a 5 or 6-membered cyclic amino-carbonyl group optionally containing an oxygen atom,
(xvii) a ureido group,
(xviii) a C₁₋₆ alkyl-ureido group,
(xix) a C₃₋₇ cycloalkyl-ureido group,
(xx) a 5- to 8-membered heterocyclyl-ureido group containing, besides carbon atoms, 1 to 3 hetero atoms selected from the group consisting of a nitrogen atom, an oxygen atom and a sulfur atom,
(xxi) a sulfamoyl group optionally substituted by C₁₋₆ alkyl,
(xxii) a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl,
(xxiii) an optionally halogenated C₁₋₆ alkyl group,
(xxiv) a C₁₋₆ alkoxy-carbonyl group,
(xxv) a halogen atom, or
(xxvi) a carboxyl group, and
ring B^{h′} is a phenyl group, a pyridyl group or a piperidyl group, each of which is optionally further substituted besides R^{5h}.

Each symbol used in the present specification is described in detail in the following.

In the present specification, unless otherwise specified, as the “halogen atom” (and “halogen” in substituent), fluorine atom, chlorine atom, bromine atom and iodine atom can be mentioned.

In the present specification, unless otherwise specified, as the “alkyl group”, a straight chain or branched alkyl group having 1 to 10 (e.g., 1 to 10, 1 to 8, 1 to 6, 2 to 6, 1 to 4) carbon atoms, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

In the present specification, unless otherwise specified, as the “C₁₋₁₀ alkyl group”, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

In the present specification, unless otherwise specified, as the “C₁₋₈ alkyl group”, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl and the like can be mentioned.

In the present specification, unless otherwise specified, as the “C₁₋₆ alkyl group”, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like can be mentioned.

In the present specification, unless otherwise specified, as the “C₂₋₆ alkyl group”, for example, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like can be mentioned.

In the present specification, unless otherwise specified, as the “C₁₋₄ alkyl group”, for example, methyl, ethyl, propyl, isopropyl, butyl, isobutyl, sec-butyl, tert-butyl and the like can be mentioned.

In the present specification, unless otherwise specified, as the “alkenyl group”, an alkenyl group having 2 to 10 (e.g., 2 to 10, 2 to 8, 2 to 6, 2 to 4) carbon atoms, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

In the present specification, unless otherwise specified, as the “C₂₋₁₀ alkenyl group”, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

In the present specification, unless otherwise specified, as the “C₂₋₈ alkenyl group”, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

In the present specification, unless otherwise specified, as the “C₂₋₆ alkenyl group”, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl and the like can be mentioned.

In the present specification, unless otherwise specified, as the “C₂₋₄ alkenyl group”, for example, ethenyl, 1-propenyl, 2-propenyl, 2-methyl-1-propenyl, 1-butenyl, 2-butenyl, 3-butenyl and the like can be mentioned.

In the present specification, unless otherwise specified, as the “alkynyl group”, an alkynyl group having 2 to 10 (e.g., 2 to 10, 2 to 8, 2 to 6, 2 to 4) carbon atoms, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

In the present specification, unless otherwise specified, as the “C₂₋₁₀ alkynyl group”, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

In the present specification, unless otherwise specified, as the “C₂₋₈ alkynyl group”, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

In the present specification, unless otherwise specified, as the “C₂₋₆ alkynyl group”, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like can be mentioned.

In the present specification, unless otherwise specified, as the “C₂₋₄ alkynyl group”, for example, ethynyl, 1-propynyl, 2-propynyl, 1-butynyl, 2-butynyl, 3-butynyl and the like can be mentioned.

In the present specification, unless otherwise specified, as the “cycloalkyl group”, a cycloalkyl group having 3 to 10 (e.g., 3 to 10, 3 to 8, 3 to 7, 3 to 6, 5 to 8) carbon atoms, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

In the present specification, unless otherwise, specified, as the “C₃₋₁₀ cycloalkyl group”, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

In the present specification, unless otherwise specified, as the “C₃₋₈ cycloalkyl group”, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and the like can be mentioned.

In the present specification, unless otherwise specified, as the “C₃₋₇ cycloalkyl group”, for example, cyclopropyl, cyclobutyl, cyclopentyl, cyclohexyl, cycloheptyl and the like can be mentioned.

In the present specification, unless otherwise specified, as the “C₅₋₈ cycloalkyl group”, for example, cyclopentyl, cyclohexyl, cycloheptyl, cyclooctyl and the like can be mentioned.

In the present specification, unless otherwise specified, as the “cycloalkenyl group”, a cycloalkenyl group having 3 to 10 carbon atoms, for example, 2-cyclopenten-1-yl, 3-cyclopenten-1-yl, 2-cyclohexen-1-yl, 3-cyclohexen-1-yl and the like can be mentioned.

In the present specification, unless otherwise specified, as the “C₃₋₁₀ cycloalkenyl group”, for example, 2-cyclopenten-1-yl, 3-cyclopenten-1-yl, 2-cyclohexen-1-yl, 3-cyclohexen-1-yl and the like can be mentioned.

In the present specification, unless otherwise specified, as the “cycloalkadienyl group”, a cycloalkadienyl group having 4 to 10 carbon atoms, for example, 2,4-cyclopentadien-1-yl, 2,4-cyclohexadien-1-yl, 2,5-cyclohexadien-1-yl and the like can be mentioned.

In the present specification, unless otherwise specified, as the “C₄₋₁₀ cycloalkadienyl group”, for example, 2,4-cyclopentadien-1-yl, 2,4-cyclohexadien-1-yl, 2,5-cyclohexadien-1-yl and the like can be mentioned.

In the present specification, unless otherwise specified, the term “aryl group” encompasses a monocyclic aryl group and a fused polycyclic aryl group. As the “aryl group”, an aryl group having 6 to 18 (e.g., 6 to 18, 6 to 14, 6 to 10) carbon atoms, for example, phenyl, naphthyl, anthryl, phenanthryl, acenaphthyl, biphenylyl and the like can be mentioned.

In the present specification, unless otherwise specified, as the “C₆₋₁₈ aryl group”, for example, phenyl, naphthyl, anthryl, phenanthryl, acenaphthyl, biphenylyl and the like can be mentioned.

In the present specification, unless otherwise specified, as the “C₆₋₁₄ aryl group”, for example, phenyl, naphthyl, anthryl, phenanthryl, acenaphthyl, biphenylyl and the like can be mentioned.

In the present specification, unless otherwise specified, as the “C₆₋₁₀ aryl group”, for example, phenyl, naphthyl and the like can be mentioned.

In the present specification, unless otherwise specified, as the “aralkyl group”, an aralkyl group having 7 to 16 carbon atoms, for example, benzyl, phenethyl, phenylpropyl, naphthylmethyl, biphenylylmethyl and the like can be mentioned.

In the present specification, unless otherwise specified, as the “C₇₋₁₆ aralkyl group”, for example, benzyl, phenethyl, phenylpropyl, naphthylmethyl, biphenylylmethyl and the like can be mentioned.

In the present specification, unless otherwise specified, as the “alkanoyl group”, an alkanoyl group having 1 to 7 (e.g., 1 to 7, 1 to 6) carbon atoms, for example, formyl, C₁₋₆ alkyl-carbonyl (e.g., acetyl, propionyl, butyryl, valeryl, pivaloyl) and the like can be mentioned.

In the present specification, unless otherwise specified, as the “C₁₋₆ alkanoyl group”, for example, formyl, C₁₋₆ alkyl-carbonyl (e.g., acetyl, propionyl, butyryl, valeryl, pivaloyl) and the like can be mentioned.

In the present specification, unless otherwise specified, as the “alkoxy group”, an alkoxy group having 1 to 6 (e.g., 1 to 6, 2 to 6, 1 to 4) carbon atoms, for example, methoxy, ethoxy, n-propoxy, isopropoxy, n-butoxy, tert-butoxy, n-pentyloxy, n-hexyloxy and the like can be mentioned.

In the present specification, unless otherwise specified, as the “C₁₋₆ alkoxy group”, for example, methoxy, ethoxy, n-propoxy, isopropoxy, n-butoxy, tert-butoxy, n-pentyloxy, n-hexyloxy and the like can be mentioned.

In the present specification, unless otherwise specified, as the “C₂₋₆ alkoxy group”, for example, ethoxy, n-propoxy, isopropoxy, n-butoxy, tert-butoxy, n-pentyloxy, n-hexyloxy and the like can be mentioned.

In the present specification, unless otherwise specified, as the “C₁₋₄ alkoxy group”, for example, methoxy, ethoxy, n-propoxy, isopropoxy, n-butoxy, tert-butoxy and the like can be mentioned.

In the present specification, unless otherwise specified, as the “alkylene”, an alkylene having 1 to 4 (e.g., 1 to 4, 1 to 3) carbon atoms, for example, —CH₂—, —CH₂CH₂—, —(CH₂)₃—, —(CH₂)₄—, —CH(CH₃)—, —C(CH₃)₂—, —CH(CH₃)CH₂—, —CH₂CH(CH₃)—, —C(CH₃)₂CH₂—, —CH₂C(CH₃)₂— and the like can be mentioned.

In the present specification, unless otherwise specified, as the “C₁₋₄ alkylene”, for example, —CH₂—, —CH₂CH₂—, —(CH₂)₃—, —(CH₂)₄—, —CH(CH₃)—, —C(CH₃)₂—, —CH(CH₃)CH₂—, —CH₂CH(CH₃)—, —C(CH₃)₂CH₂—, —CH₂C(CH₃)₂— and the like can be mentioned.

In the present specification, unless otherwise specified, as the “C₁₋₃ alkylene”, for example, —CH₂—, —CH₂CH₂—, —(CH₂)₃—, —(CH₂)₄—, —CH(CH₃)—, —C(CH₃)₂—, —CH(CH₃)CH₂—, —CH₂CH(CH₃)— and the like can be mentioned.

In the present specification, unless otherwise specified, as the “hydrocarbon group” of the “optionally substituted hydrocarbon group”, for example, an alkyl group, an alkenyl group, an alkynyl group, a cycloalkyl group, a cycloalkenyl group, a cycloalkadienyl group, an aryl group, an aralkyl group, an arylalkenyl group, a cycloalkyl-alkyl group and the like can be mentioned. Of these, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₆ aralkyl group, a C₈₋₁₃ arylalkenyl group, a C₃₋₁₀ cycloalkyl-C₁₋₆ alkyl group and the like are preferable.

The above-mentioned C₃₋₁₀ cycloalkyl group, C₃₋₁₀ cycloalkenyl group and C₄₋₁₀ cycloalkadienyl group are each optionally condensed with a benzene ring, and as such a fused ring group, for example, indanyl, dihydronaphthyl, tetrahydronaphthyl, fluorenyl and the like can be mentioned. In addition, as the above-mentioned hydrocarbon group, a crosslinked hydrocarbon group such as norbornanyl, adamantyl and the like, and the like can also be mentioned.

As the C₈₋₁₃ arylalkenyl group, for example, styryl and the like can be mentioned.

As the C₃₋₁₀ cycloalkyl-C₁₋₆ alkyl group, for example, cyclopropylmethyl, cyclohexylmethyl and the like can be mentioned.

The above-mentioned C₁₋₁₀ alkyl group, C₂₋₁₀ alkenyl group and C₂₋₁₀ alkynyl group, which are exemplarily recited as the “hydrocarbon group”, each optionally has 1 to 3 substituents at substitutable positions.

As such substituents, for example,
(1) a C₃₋₁₀ cycloalkyl group (e.g., cyclopropyl, cyclohexyl) optionally substituted by 1 to 3 substituents selected from the group consisting of
halogen;
hydroxy;
carboxyl;
sulfo;
cyano;
azido;
nitro;
nitroso;
optionally halogenated C₁₋₄ alkyl;
optionally halogenated C₂₋₄ alkenyl;
optionally halogenated C₂₋₄ alkynyl;
C₃₋₇ cycloalkyl;
C₆₋₁₄ aryl;
C₇₋₁₆ aralkyl;
formyl;
optionally halogenated C₁₋₆ alkyl-carbonyl;
optionally halogenated C₁₋₆ alkoxy-carbonyl;
optionally halogenated C₁₋₆ alkylsulfonyl;
carbamoyl;
carbamoyl mono- or di-substituted by optionally halogenated C₁₋₆ alkyl;
mono- or di-C₆₋₁₄ aryl-carbamoyl;
thiocarbamoyl optionally mono- or di-substituted by optionally halogenated C₁₋₆ alkyl;
ureido optionally mono- or di-substituted by optionally halogenated C₁₋₆ alkyl;
mono- or di-C₆₋₁₄ aryl-ureido;
sulfamoyl optionally mono- or di-substituted by optionally halogenated C₁₋₆ alkyl;
optionally halogenated C₁₋₆ alkoxy;
optionally halogenated C₂₋₆ alkenyloxy;
C₃₋₁₀ cycloalkyloxy;
C₇₋₁₆ aralkyloxy;
C₆₋₁₄ aryloxy;
C₁₋₆ alkyl-carbonyloxy;
C₃₋₁₀ cycloalkyl-C₁₋₆ alkoxy;
C₁₋₆ alkylsulfonyloxy;
mercapto;
optionally halogenated C₁₋₆ alkylthio;
C₇₋₁₆ aralkylthio;
C₆₋₁₄ arylthio;
C₁₋₆ alkylsulfinyl;
oxo;
C₁₋₃ alkylenedioxy (e.g., methylenedioxy, ethylenedioxy);
hydroxyimino optionally substituted by C₁₋₆ alkyl;
and the like (Substituent Group S);
(2) a C₆₋₁₄ aryl group (e.g., phenyl, naphthyl) optionally substituted by 1 to 3 substituents selected from
Substituent Group S;
(3) a heterocyclic group optionally substituted by 1 to 3 substituents selected from Substituent Group S;
(4) an amino group optionally substituted by 1 or 2 substituents selected from the group consisting of C₁₋₆ alkyl optionally substituted by substituent(s) selected from the group consisting of halogen, hydroxy, C₃₋₇ cycloalkyl, C₁₋₆ alkylsulfonyl, C₁₋₆ alkoxy and the like;
optionally halogenated C₂₋₄ alkenyl;
optionally halogenated C₂₋₄ alkynyl;
C₃₋₇ cycloalkyl;
C₆₋₁₄ aryl;
C₇₋₁₆ aralkyl;
4 to 7-membered (preferably 5 or 6-membered) heterocyclic group (e.g., non-aromatic heterocyclic group such as morpholinyl and the like) containing, as a ring-constituting atom besides carbon atoms, 1 to 4 hetero atoms selected from the group consisting of an oxygen atom, a sulfur atom and a nitrogen atom;
formyl;
C₁₋₆ alkyl-carbonyl optionally substituted by substituent(s) selected from the group consisting of halogen, hydroxy, C₃₋₇ cycloalkyl, C₁₋₆ alkylsulfonyl, C₁₋₆ alkoxy and the like;
C₁₋₆ alkoxy-carbonyl;
C₆₋₁₄ aryl-carbonyl (e.g., benzoyl);
C₇₋₁₆ aralkyl-carbonyl (e.g., benzylcarbonyl, phenethylcarbonyl);
C₃₋₇ cycloalkyl-carbonyl;
C₁₋₆ alkyl-carbamoyl (e.g., methylaminocarbonyl, ethylaminocarbonyl);
C₆₋₁₄ aryl-carbamoyl (e.g., phenylaminocarbonyl, 1-naphthylaminocarbonyl, 2-naphthylaminocarbonyl);
C₇₋₁₆ aralkyl-carbamoyl (e.g., benzylaminocarbonyl);
C₁₋₆ alkylsulfonyl (e.g., methylsulfonyl, ethylsulfonyl, isopropylsulfonyl);
C₆₋₁₄ arylsulfonyl (e.g., benzenesulfonyl, toluenesulfonyl, 1-naphthalenesulfonyl, 2-naphthalenesulfonyl);
C₇₋₁₆ aralkylsulfonyl (e.g., benzylsulfonyl);
and the like (Substituent Group T);
(5) an amidino group;
(6) an optionally formylated or halogenated C₁₋₆ alkyl-carbonyl group;
(7) an optionally halogenated C₁₋₆ alkoxy-carbonyl group;
(8) an optionally halogenated C₁₋₆ alkylsulfonyl group (e.g., methylsulfonyl);
(9) a carbamoyl group optionally substituted by 1 or 2 substituents selected from Substituent Group T;
(10) a thiocarbamoyl group optionally mono- or di-substituted by optionally halogenated C₁₋₆ alkyl group;
(11) a ureido group optionally substituted by 1 or 2 substituents selected from Substituent Group T;
(12) a sulfamoyl group optionally substituted by 1 or 2 substituents selected from Substituent Group T;
(13) a carboxyl group;
(14) a hydroxy group;
(15) a C₁₋₆ alkoxy group optionally substituted by 1 to 3 substituents selected from the group consisting of halogen, carboxyl, C₁₋₆ alkoxy and C₁₋₆ alkoxy-carbonyl;
(16) an optionally halogenated C₂₋₆ alkenyloxy group (e.g., ethenyloxy);
(17) a C₃₋₁₀ cycloalkyloxy group (e.g., cyclohexyloxy);
(18) a C₇₋₁₆ aralkyloxy group (e.g., benzyloxy);
(19) a C₆₋₁₄ aryloxy group (e.g., phenyloxy, naphthyloxy);
(20) a C₁₋₆ alkyl-carbonyloxy group (e.g., acetyloxy, tert-butylcarbonyloxy);
(21) a mercapto group;
(22) an optionally halogenated C₁₋₆ alkylthio group (e.g., methylthio, ethylthio);
(23) a C₇₋₁₆ aralkylthio group (e.g., benzylthio);
(24) a C₆₋₁₄ arylthio group (e.g., phenylthio, naphthylthio);
(25) a sulfo group;
(26) a cyano group;
(27) an azido group;
(28) a nitro group;
(29) a nitroso group;
(30) a halogen atom;
(31) a C₁₋₆ alkylsulfinyl group (e.g., methylsulfinyl);
(32) an oxo group;
(33) a C₃₋₁₀ cycloalkyl-C₁₋₆ alkoxy group (e.g., cyclopropylmethoxy);
(34) a C₁₋₃ alkylenedioxy group (e.g., methylenedioxy, ethylenedioxy);
(35) a hydroxyimino group optionally substituted by C₁₋₆ alkyl;
and the like (Substituent Group U) can be mentioned. When the number of the substituents is not less than 2, respective substituents may be the same or different.

The above-mentioned C₃₋₁₀ cycloalkyl group, C₃₋₁₀ cycloalkenyl group, C₄₋₁₀ cycloalkadienyl group, C₆₋₁₄ aryl group, C₇₋₁₆ aralkyl group, C₈₋₁₃ arylalkenyl group and C₃₋₁₀ cycloalkyl-C₁₋₆ alkyl group, which are exemplarily recited as the “hydrocarbon group”, each optionally have 1 to 3 substituents at substitutable positions.

As such substituents, for example,
(1) a substituent selected from Substituent Group U;
(2) a C₁₋₁₀ alkyl group optionally substituted by 1 to 3 substituents selected from Substituent Group U;
(3) a C₂₋₁₀ alkenyl group (e.g., ethenyl, 1-propenyl) optionally substituted by 1 to 3 substituents selected from Substituent Group U;
(4) a C₇₋₁₆ aralkyl group (e.g., benzyl) optionally substituted by 1 to 3 substituents selected from Substituent Group U;
and the like (Substituent Group V) can be mentioned. When the number of the substituents is not less than 2, respective substituents may be the same or different.

In the present specification, unless otherwise specified, as the “heterocyclic group” of the “optionally substituted heterocyclic group”, an aromatic heterocyclic group and a non-aromatic heterocyclic group can be mentioned.

As the aromatic heterocyclic group, for example, a 4 to 7-membered (preferably 5 or 6-membered) monocyclic aromatic heterocyclic group containing, as a ring-constituting atom besides carbon atoms, 1 to 4 hetero atoms selected from the group consisting of an oxygen atom, a sulfur atom and a nitrogen atom and a fused aromatic heterocyclic group can be mentioned. As the fused aromatic heterocyclic group, for example, a group derived from a fused ring wherein a ring corresponding to such 4- to 7-membered monocyclic aromatic heterocyclic group, and 1 or 2 rings selected from the group consisting of a 5- or 6-membered ring containing 1 or 2 nitrogen atoms, a 5-membered ring containing one sulfur atom, a benzene ring and the like are condensed, and the like can be mentioned.

As preferable examples of the aromatic heterocyclic group,
monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,2,4-triazin-1-yl, 1,2,4-triazin-3-yl) and the like;
fused aromatic heterocyclic groups such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like;
and the like can be mentioned.

As the non-aromatic heterocyclic group, for example, a 4 to 7-membered (preferably 5 or 6-membered) monocyclic non-aromatic heterocyclic group containing, as a ring-constituting atom besides carbon atoms, 1 to 4 hetero atoms selected from the group consisting of an oxygen atom, a sulfur atom and a nitrogen atom and a fused non-aromatic heterocyclic group can be mentioned. As the fused non-aromatic heterocyclic group, for example, a group derived from a fused ring wherein a ring corresponding to such 4- to 7-membered monocyclic non-aromatic heterocyclic group, and 1 or 2 rings selected from the group consisting of a 5- or 6-membered ring containing 1 or 2 nitrogen atoms, a 5-membered ring containing one sulfur atom, a benzene ring and the like are condensed, and the like can be mentioned.

As preferable examples of the non-aromatic heterocyclic group,
monocyclic non-aromatic heterocyclic groups such as oxetanyl (e.g., 2-oxetanyl, 3-oxetanyl), pyrrolidinyl (e.g., 1-pyrrolidinyl, 2-pyrrolidinyl), piperidinyl (e.g., piperidino, 2-piperidinyl, 3-piperidinyl, 4-piperidinyl), morpholinyl (e.g., morpholino), thiomorpholinyl (e.g., thiomorpholino), piperazinyl (e.g., 1-piperazinyl, 2-piperazinyl, 3-piperazinyl), hexamethyleniminyl (e.g., hexamethylenimin-1-yl), oxazolidinyl (e.g., oxazolidin-2-yl), thiazolidinyl (e.g., thiazolidin-2-yl), imidazolidinyl (e.g., imidazolidin-2-yl, oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tetrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxidotetrahydrothiopyranyl (e.g., 1-oxidotetrahydrothiopyran-4-yl), 1,1-dioxidotetrahydrothiopyranyl (e.g., 1,1-dioxidotetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl) and the like;
fused non-aromatic heterocyclic groups such as dihydroindolyl (e.g., 2,3-dihydro-1H-indol-1-yl), dihydroisoindolyl (e.g., 1,3-dihydro-2H-isoindol-2-yl), dihydrobenzofuranyl (e.g., 2,3-dihydro-1-benzofuran-5-yl), dihydrobenzodioxinyl (e.g., 2,3-dihydro-1,4-benzodioxinyl), dihydrobenzodioxepinyl (e.g., 3,4-dihydro-2H-1,5-benzodioxepinyl), tetrahydrobenzofuranyl (e.g., 4,5,6,7-tetrahydro-1-benzofuran-3-yl), chromenyl (e.g., 4H-chromen-2-yl, 2H-chromen-3-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquinolinyl (e.g., 1,2,3,4-tetrahydroisoquinolin-4-yl), dihydrophthalazinyl (e.g., 1,4-dihydrophthalazin-4-yl) and the like;
and the like can be mentioned.

The “heterocyclic group” of the “optionally substituted heterocyclic group” optionally has 1 to 3 substituents at substitutable positions. As such substituents, for example, substituents selected from Substituent Group V can be mentioned. When the number of the substituents is not less than 2, respective substituents may be the same or different.

In the present specification, unless otherwise specified, as the “aliphatic hydrocarbon group” of the “optionally substituted aliphatic hydrocarbon group”, a linear or branched aliphatic hydrocarbon group having 1 to 10 carbon atoms (preferably, 1 to 8 carbon atoms) can be mentioned. As the “aliphatic hydrocarbon group”, for example, a C₁₋₁₀ alkyl group, a C₂₋₁₀ alkenyl group, a C₂₋₁₀ alkynyl group and a C₃₋₁₀ cycloalkyl group can be mentioned (each group is as defined above).

The “aliphatic hydrocarbon group” is optionally substituted by substituent(s) selected from Substituent Group V, particularly, 1 to 3 substituents selected from the group consisting of halogen, hydroxy, C₁₋₄ alkoxy, C₁₋₄ alkyl-carbonyl, carboxy, C₁₋₄ alkoxy-carbonyl, cyano, carbamoyl, sulfamoyl, nitro, amino, C₁₋₄ alkyl-carbonylamino, C₁₋₄ alkoxy-carbonylamino and C₁₋₄ alkylsulfonylamino. When the number of the substituents is not less than 2, respective substituents may be the same or different.

In the present specification, unless otherwise specified, as the “acyl group”, for example, —COR^{Y1}, —CO—OR^{Y1}, —SO₂R^{Y1}, —SOR^{Y1}, —PO(OR^{Y1})(OR^{Y2}) (wherein R^{Y1} and R^{Y2} are the same or different and each is a hydrogen atom, an optionally substituted hydrocarbon group, or an optionally substituted heterocyclic group), and the like can be mentioned.

In the present specification, unless otherwise specified, the “amino group” of the “optionally substituted amino group”, the “carbamoyl group” of the “optionally substituted carbamoyl group”, the “ureido group” of the “optionally substituted ureido group” and the “sulfamoyl group” of the “optionally substituted sulfamoyl group” optionally have 1 or 2 substituents at substitutable position(s). As such substituents, for example, an optionally substituted hydrocarbon group, an optionally substituted heterocyclic group, an acyl group and the like can be mentioned. Of these, 1 or 2 substituents selected from Substituent Group T are preferable. When the number of the substituents is not less than 2, respective substituents may be the same or different.

When the nitrogen atom constituting the above-mentioned amino group, carbamoyl group, ureido group or sulfamoyl group is substituted by two substituents, these substituents may in combination form, together with the adjacent nitrogen atom, a nitrogen-containing heterocycle. As the “nitrogen-containing heterocycle”, for example, a 3 to 8-membered nitrogen-containing heterocycle containing, as a ring-constituting atom besides carbon atoms, at least one nitrogen atom and optionally further containing one or two heteroatoms selected from the group consisting of an oxygen atom, a sulfur atom and a nitrogen atom can be mentioned. As preferable examples of the nitrogen-containing heterocycle, a 5 or 6-membered cyclic amine optionally containing an oxygen atom (e.g., 1-pyrrolidine, piperidine, 1-piperazine, morpholine) can be mentioned.

In the present specification, unless otherwise specified, the “imino group” of the “optionally substituted imino group” optionally has 1 or 2 substituents at substitutable position(s). As such substituents, for example, an optionally substituted hydrocarbon group, an optionally substituted heterocyclic group, an acyl group and the like can be mentioned. Of these, substituents selected from Substituent Group T are preferable. When the number of the substituents is not less than 2, respective substituents may be the same or different.

In the present specification, unless otherwise specified, as the “optionally substituted group bonded via a carbon atom, a nitrogen atom or an oxygen atom”, a group represented by the formula: —X^{x}—R^{x}, an amino group and a hydroxy group can be mentioned.

In the above-mentioned formula, X^{x} is a bond, —NR^{Y}— (wherein R^{Y} is a hydrogen atom or a C₁₋₆ alkyl group), or —O—.

In the above-mentioned formula, R^{x} is a cyano group, or a C₁₋₈ alkyl group, a C₂₋₈ alkenyl group, a C₂₋₈ alkynyl group, a carbamoyl group, a C₁₋₈ alkyl-carbonyl group, a C₃₋₈ cycloalkyl group, a C₆₋₁₈ aryl group, a C₆₋₁₈ aryl-C₁₋₄ alkyl group, a C₆₋₁₈ aryl-carbonyl group, a C₆₋₁₈ aryl-C₁₋₄ alkyl-carbonyl group, a heterocyclic group, a heterocyclyl-C₁₋₄ alkyl group, a heterocyclylcarbonyl group or a heterocyclyl-C₁₋₄ alkyl-carbonyl group, each of which is optionally substituted.

In the above-mentioned formula, the “C₁₋₈ alkyl group”, “C₂₋₈ alkenyl group”, “C₂₋₈ alkynyl group”, “carbamoyl group”, “C₁₋₈ alkyl-carbonyl group”, “C₃₋₈ cycloalkyl group”, “C₆₋₁₈ aryl group”, “C₆₋₁₈ aryl-C₁₋₄ alkyl group”, “C₆₋₁₈ aryl-carbonyl group”, “C₆₋₁₈ aryl-C₁₋₄ alkyl-carbonyl group”, “heterocyclic group”, “heterocyclyl-C₁₋₄ alkyl group”, “heterocyclylcarbonyl group” and “heterocyclyl-C₁₋₄ alkyl-carbonyl group” for R^{x} are optionally substituted by one or more (preferably 1 to 5, more preferably 1 to 3) substituents selected from, for example, the following group (Substituent Group X)
(a) a halogen atom,
(b) an oxo group,
(c) an optionally halogenated C₁₋₄ alkyl group,
(d) —(CH₂)ₘ-Q^{x} group,
(e) —(CH₂)ₘ—Z^{1x}-optionally halogenated C₁₋₄ alkyl group,
(f) —(CH₂)ₘ—Z^{1x}—C₃₋₈ cycloalkyl group,
(g) —(CH₂)ₘ—Z^{2x}—(CH₂)ₙ-Q^{x} group,
(h) —(CH₂)ₘ—Z^{2x}—(CH₂)ₙ—Z^{1x}-optionally halogenated C₁₋₄ alkyl group,
(i) —(CH₂)ₘ—Z^{2x}—(CH₂)ₙ—Z^{1x}—C₃₋₈ cycloalkyl group,
(j) —(CH₂)ₘ—Z^{1x}-optionally substituted heterocyclic group (preferably, the heterocyclic group is a 5- to 8-membered heterocyclic group containing 1 to 3 hetero atoms selected from the group consisting of a nitrogen atom, an oxygen atom and optionally oxidized sulfur atom),
(k) —(CH₂)ₘ—Z^{2x}—C₁₋₄ alkoxy group, and
(l) —(CH₂)ₘ—Z^{2x}—(CH₂)ₙ—Z^{1x}—(CH₂)ₙ—Z^{1x}—C₁₋₄ alkyl group.

R^{Y} is preferably a hydrogen atom or methyl, particularly preferably a hydrogen atom.

In the above-mentioned formula,
m is an integer of 0 to 4,
n is an integer of 1 to 4,
Q^{x} is hydroxy, carboxy, cyano, nitro, —NR^{1x}R^{2x}, —CONR^{1x}R^{2x} or —SO₂NR^{1x}R^{2x},
Z^{1x} is —O—, —CO—, —C(OH)R^{3x}—, —C(═N—OR^{3x})—, —S—, —SO—, —SO₂—, —N(COR^{3x})—, —N(CO₂R^{4x})—, —N(SO₂R^{4x})—, —CO—O—, —O—CO—, —CO—NR^{3x}—, —NR^{3x}—CO—, —NR^{3x}—CO₂—, —NR^{3x}—CO—NH—, —NR^{3x}—SO₂— or —NR^{3x}—C(═NH)—NH—,
Z^{2x} is —O—, —CO—, —C(OH)R^{3x}—, —C(═N—OR^{3x})—, —S—, —SO—, —SO₂—, —NR^{3x}—, —N(COR^{3x})—, —N(CO₂R^{4x})—, —N(SO₂R^{4x})—, —CO—O—, —O—CO—, —CO—NR^{3x}—, —NR^{3x}—CO—, —NR^{3x}—CO₂—, —NR^{3x}—CO—NH—, —NR^{3x}—C(═NH)—NH—, —NR^{3x}—SO₂— or —SO₂—NR^{3x}—.

In the above-mentioned formula, —(CH₂)ₘ— and —(CH₂)ₙ— are optionally substituted by one or more (preferably 1 to 5, more preferably 1 to 3) substituents selected from, for example, the group consisting of halogen, optionally halogenated C₁₋₄ alkyl and hydroxy, and when m or n is not less than 2, a subset —CH₂CH₂— of —(CH₂)ₘ— or —(CH₂)ₙ— is optionally replaced by —CH═CH— or —C≡C—.

In the above-mentioned formula, R^{1x} and R^{2x} are the same or different and each is a hydrogen atom or a C₁₋₄ alkyl, or R^{1x} and R^{2x} are optionally bonded to form a ring together with the nitrogen atom. In the above-mentioned formula, R^{3x} is a hydrogen atom or a C₁₋₄ alkyl, and R^{4x} is a C₁₋₄ alkyl.

When R^{1x} and R^{2x} are bonded to form a ring together with the nitrogen atom, as the nitrogen-containing heterocycle, for example, 3 to 8-membered (preferably 5 or 6-membered) saturated or unsaturated (preferably saturated) aliphatic heterocyclic groups such as azetidine, pyrrolidine, piperidine, homopiperidine, heptamethylenimine, morpholine, thiomorpholine, piperazine, homopiperazine and the like can be mentioned.

In the present specification, unless otherwise specified, as the “optionally substituted group bonded via a carbon atom or a sulfur atom”, a C₁₋₈ alkyl group, a C₂₋₈ alkenyl group, a C₂₋₈ alkynyl group, a carbamoyl group, a C₁₋₈ alkyl-carbonyl group, a C₁₋₈ alkylthio group, a C₁₋₈ alkylsulfonyl group, a C₃₋₈ cycloalkyl group, a C₆₋₁₈ aryl group, a C₆₋₁₈ aryl-C₁₋₄ alkyl group, a C₆₋₁₈ aryl-carbonyl group, a C₆₋₁₈ aryl-C₁₋₄ alkyl-carbonyl group, a C₆₋₁₈ arylthio group, a C₆₋₁₈ arylsulfonyl group, a heterocyclic group, a heterocyclyl-C₁₋₄ alkyl group, a heterocyclylcarbonyl group, a heterocyclyl-C₁₋₄ alkyl-carbonyl group, a heterocyclylthio group and a heterocyclyl-C₁₋₄ alkylthio group, each of which is optionally substituted, and the like can be mentioned.

The “C₁₋₈ alkyl group”, “C₂₋₈ alkenyl group”, “C₂₋₈ alkynyl group”, “carbamoyl group”, “C₁₋₈ alkyl-carbonyl group”, “C₁₋₈ alkylthio group”, “C₁₋₈ alkylsulfonyl group”, “C₃₋₈ cycloalkyl group”, “C₆₋₁₈ aryl group”, “C₆₋₁₈ aryl-C₁₋₄ alkyl group”, “C₆₋₁₈ aryl-carbonyl group”, “C₆₋₁₈ aryl-C₁₋₄ alkyl-carbonyl group”, “C₆₋₁₈ arylthio group”, “C₆₋₁₈ arylsulfonyl group”, “heterocyclic group”, “heterocyclyl-C₁₋₄ alkyl group”, “heterocyclylcarbonyl group”, “heterocyclyl-C₁₋₄ alkyl-carbonyl group”, “heterocyclylthio group” and “heterocyclyl-C₁₋₄ alkylthio group” are optionally substituted by one or more (preferably 1 to 5, more preferably 1 to 3) substituents selected from, for example, Substituent Group X.

### [Compound (Ia)]

The present invention provides a compound represented by the formula (Ia) or a salt thereof (in the present specification, hereinafter sometimes to be abbreviated as “compound (Ia)”).

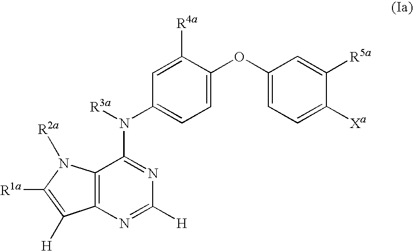

wherein each symbol is as defined above.

R^{2a} is preferably a C₁₋₆ alkyl group (particularly, an ethyl group) substituted by a group represented by the formula “—NR^{6aa}—CO—CR^{7a}R^{8a}—SO₂—C₁₋₄ alkyl”.

In the formula, R^{6aa} is a hydrogen atom or a methyl group, and R^{7a} and R^{8a} are the same or different and each is a hydrogen atom or a methyl group. R^{7a} and R^{8a} are preferably methyl groups.

R^{3a} is preferably a hydrogen atom.

As the “halogen atom” for R^{4a}, a chlorine atom is preferable. As the “C₁₋₆ alkyl group” for R^{4a}, a methyl group is preferable. R^{4a} is preferably a chlorine atom or a methyl group.

As the “halogen atom” for R^{5a}, a fluorine atom and a chlorine atom are preferable. As the “C₁₋₆ alkyl group” for R^{5a}, a methyl group is preferable. R^{5a} is preferably a fluorine atom, a chlorine atom or a methyl group.

As the “halogen atom” for X^{a}, a fluorine atom is preferable. X^{a} is preferably a hydrogen atom or a fluorine atom, more preferably a hydrogen atom.

As preferable embodiment of compound (Ia), compound (Ia) wherein
R^{1a} is a hydrogen atom,
R^{2a} is a C₁₋₆ alkyl group (particularly, an ethyl group) substituted by a group represented by —NR^{6aa}—CO—CR^{7a}R^{8a}—SO₂—C₁₋₄ alkyl
wherein R^{6aa} is a hydrogen atom or a methyl group, R^{7a} and R^{8a} are the same or different and each is a hydrogen atom or a methyl group,
R^{3a} is a hydrogen atom,
R^{4a} is a chlorine atom or a methyl group,
R^{5a} is a fluorine atom, a chlorine atom or a methyl group, and
X^{a} is a hydrogen atom or fluorine atom (preferably, a hydrogen atom),
can be mentioned.

As more preferable embodiment of compound (Ia), compound (Ia) wherein
R^{1a} is a hydrogen atom,
R^{2a} is a C₁₋₆ alkyl group (particularly, an ethyl group) substituted by a group represented by —NR^{6aa}—CO—CR^{7a}R^{8a}—SO₂—C₁₋₄ alkyl
wherein R^{6aa} is a hydrogen atom or a methyl group, R^{7a} and
R^{8a} are methyl groups,
R^{3a} is a hydrogen atom,
R^{4a} is a chlorine atom or a methyl group,
R^{5a} is a fluorine atom, a chlorine atom or a methyl group, and
X^{a} is a hydrogen atom or fluorine atom (preferably, a hydrogen atom),
can be mentioned.

As compound (Ia), particularly preferably,
N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide,
N-[2-(4-{[3-chloro-4-(3-chlorophenoxy)phenyl]amino}-5H-pyrrolo[3,2-d]pyrimidin-5-yl)ethyl]-2-(ethylsulfonyl)acetamide,
N-[2-(4-{[3-chloro-4-(3-chlorophenoxy)phenyl]amino}-5H-pyrrolo[3,2-d]pyrimidin-5-yl)ethyl]-N,2-dimethyl-2-(methylsulfonyl)propanamide,
N-[2-(4-{[3-chloro-4-(3-methylphenoxy)phenyl]amino}-5H-pyrrolo[3,2-d]pyrimidin-5-yl)ethyl]-2-(methylsulfonyl)acetamide,
N-[2-(4-{[3-chloro-4-(3-fluorophenoxy)phenyl]amino}-5H-pyrrolo[3,2-d]pyrimidin-5-yl)ethyl]-2-(methylsulfonyl)acetamide, and
N-[2-(4-{[4-(3-chlorophenoxy)-3-methylphenyl]amino}-5H-pyrrolo[3,2-d]pyrimidin-5-yl)ethyl]-2-methyl-2-(methylsulfonyl)propanamide,
and salts and hydrates thereof can be mentioned.

### [Compound (Ib)]

The present invention provides also a compound represented by the formula (Ib) or a salt thereof (in the present specification, hereinafter sometimes to be abbreviated as “compound (Ib)”).

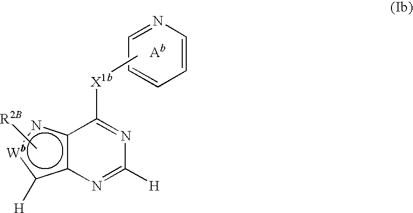

wherein each symbol is as defined above.

In the above-mentioned formula (Ib), the “pyridine ring” of the “optionally substituted pyridine ring” for ring A^{b} is optionally substituted by, for example, a group represented by the formula: —Y^{2b}B^{b′}. Y^{2b} is a bond, —O—, —O—(C₁₋₃ alkylene)-, —NR^{Zb}— (wherein R^{Zb} is a hydrogen atom or a C₁₋₆ alkyl group), or —S—, and B^{b′} is a C₆₋₁₈ aryl group (preferably, a C₆₋₁₄ aryl group, more to preferably a phenyl group), a heterocyclic group (preferably, a 5 or 6-membered heterocyclic group, more preferably a pyridyl group or a piperidyl group), a C₃₋₈ cycloalkyl group (preferably, a cyclohexyl group), a carbamoyl group, a ureido group, a C₆₋₁₈ aryl-carbonyl group or a C₆₋₁₈ aryl-C₁₋₄ alkyl-carbonyl group, each of which is optionally substituted.

Y^{2b} is preferably a bond, —O— or —OCH₂—, more preferably —O— or —OCH₂—, particularly preferably —O—.

The “C₆₋₁₈ aryl group”, “heterocyclic group”, “C₃₋₈ cycloalkyl group”, “carbamoyl group”, “ureido group”, “C₆₋₁₈ aryl-carbonyl group” and “C₆₋₁₈ aryl-C₁₋₄ alkyl-carbonyl group” of the “C₆₋₁₈ aryl group, heterocyclic group, C₃₋₈ cycloalkyl group, carbamoyl group, ureido group, C₆₋₁₈ aryl-carbonyl group or C₆₋₁₈ aryl-C₁₋₄ alkyl-carbonyl group, each of which is optionally substituted” for B^{b′} each optionally have 1 to 5, the same or different substituents at any substitutable positions. As the substituents, substituents similar to the above-mentioned Substituent Group V can be mentioned. Of these, optionally halogenated C₁₋₆ alkyl, optionally halogenated C₁₋₆ alkoxy, C₁₋₆ alkyl-carbamoyl and halogen are preferable.

B^{b′} is preferably an optionally substituted C₆₋₁₄ aryl group, more preferably a phenyl group optionally substituted by substituent(s) selected from the group consisting of optionally halogenated C₁₋₆ alkyl, optionally halogenated C₁₋₆ alkoxy, C₁₋₆ alkyl-carbamoyl and halogen (preferably a phenyl group optionally substituted by substituent(s) selected from the group consisting of optionally halogenated C₁₋₆ alkyl, optionally halogenated C₁₋₆ alkoxy and C₁₋₆ alkyl-carbamoyl), particularly preferably a phenyl group optionally substituted at the 3-position by substituent(s) selected from the group consisting of optionally halogenated C₁₋₆ alkyl, optionally halogenated C₁₋₆ alkoxy, C₁₋₆ alkyl-carbamoyl and halogen (preferably, a phenyl group optionally substituted at the 3-position by substituent(s) selected from the group consisting of optionally halogenated C₁₋₆ alkyl, optionally halogenated C₁₋₆ alkoxy and C₁₋₆ alkyl-carbamoyl).

The “pyridine ring” of the “optionally substituted pyridine ring” for ring A^{b} optionally further has, besides the group represented by the formula: —Y^{2b}—B^{b′}, 1 to 3, the same or different substituents at any substitutable positions. As the substituents, substituents similar to the above-mentioned Substituent Group V can be mentioned. Of these, halogen and methyl are preferable.

Ring A^{b} is preferably a pyridine ring optionally further substituted, besides the group represented by the formula: —Y^{2b}—B^{b′}, by substituent(s) selected from the group consisting of halogen and methyl, more preferably a pyridine ring optionally further substituted, besides the group represented by the formula: —Y^{2b}—B^{b′}, by halogen.

As the “aliphatic hydrocarbon group” of the “optionally substituted aliphatic hydrocarbon group” for R^{3b}, a C₁₋₆ alkyl group is preferable.

The “C₁₋₄ alkylene” and “—O—(C₁₋₄ alkylene)-” of the “C₁₋₄ alkylene or —O—(C₁₋₄ alkylene)-, each of which is optionally substituted” for Y^{1b} are optionally substituted by 1 to 3 substituent selected from the group consisting of halogen, hydroxy, C₁₋₄ alkoxy, C₁₋₄ alkyl-carbonyl, carboxy, C₁₋₄ alkoxy-carbonyl, cyano, carbamoyl, sulfamoyl, nitro, amino, C₁₋₄ alkyl-carbonylamino, C₁₋₄ alkoxy-carbonylamino and C₁₋₄ alkylsulfonylamino.

X^{1b} is preferably —NR^{3b}—. In the formula, R^{3b} is preferably a hydrogen, atom or a C₁₋₆ alkyl group, more preferably a hydrogen atom.

W^{b} is preferably C(R^{1b}).

As the “optionally substituted group bonded via a carbon atom, a nitrogen atom or an oxygen atom” for R^{1b}, a cyano group and an optionally substituted C₁₋₈ alkyl group are preferable. As the C₁₋₆ alkyl group, a C₁₋₆ alkyl group is preferable.

As the substituents for the alkyl group, substituents similar to the above-mentioned Substituent Group X can be mentioned. Of these, halogen is preferable.

R^{1b} is preferably a hydrogen atom, a halogen atom, a cyano group or an optionally halogenated C₁₋₆ alkyl group, more preferably a hydrogen atom.

As the “optionally substituted group bonded via a carbon atom or a sulfur atom” for R^{2b}, an optionally substituted C₁₋₆ alkyl group is preferable. As the C₁₋₈ alkyl group, a C₁₋₆ alkyl group is preferable.

As the substituents for the alkyl group, substituents similar to the above-mentioned Substituent Group X can be mentioned, preferably, substituent(s) selected from the group consisting of
(i) —NR^{6ba}—CO—(CH₂)ₙ₁—SO₂—C₁₋₄ alkyl
wherein R^{6ba} is a hydrogen atom or a methyl group, n1 is an integer of 1 to 4, and —(CH₂)ₙ₁— is optionally substituted by C₁₋₄ alkyl,
(ii) —NR^{6bb}—CO—(CH₂)ₙ₂—OH
wherein R^{6bb} is a hydrogen atom or a methyl group, n2 is an integer of 1 to 4, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl, and
(iv) hydroxy
can be used.

As the “ring structure” of the “optionally substituted ring structure” formed by R^{3b} bonded to the carbon atom on the pyridine ring for ring A^{b}, a saturated or unsaturated (preferably saturated) 4 to 8-membered (preferably 5 or 6-membered) nitrogen-containing heterocycle can be mentioned.

Specifically,

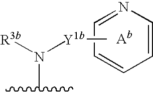

wherein each symbol is as defined above, is, for example,

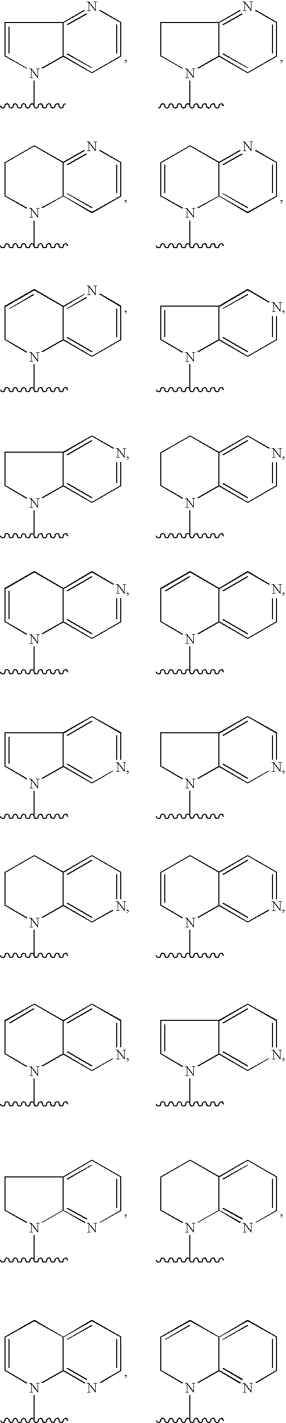

and the like.

The “ring structure” optionally has 1 to 5 (preferably 1 to 3, more preferably 1 or 2), the same or different substituents at any substitutable positions. As the substituents, substituents similar to the above-mentioned Substituent Group V can be mentioned.

R^{1b} and R^{2b} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{1b} and R^{2b} bonded to each other, for example,

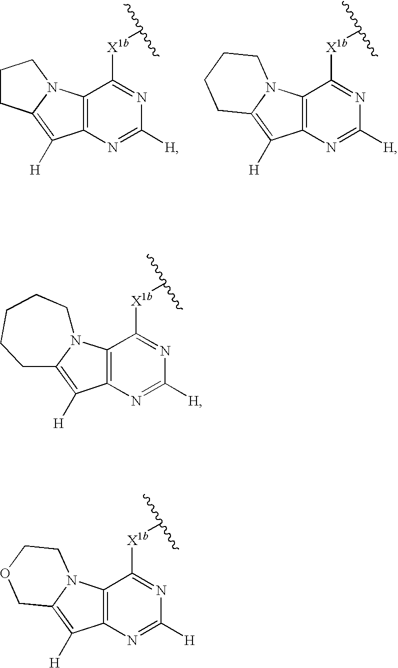

wherein each symbol is as defined above,
and the like can be mentioned.

R^{2b} and R^{3b} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring' structure” formed by R^{2b} and R^{3b} bonded to each other, for example,

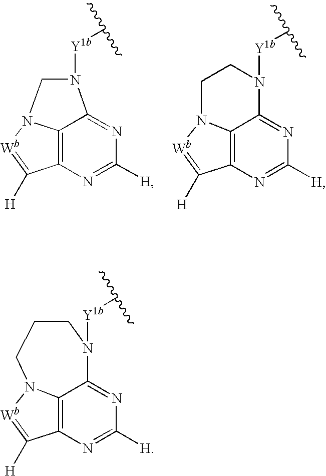

wherein each symbol is as defined above,
and the like can be mentioned.

The “ring structure” of the “optionally substituted ring structure” formed by R^{1b} and R^{2b}, or R^{2b} and R^{3b} optionally has 1 to 5 (preferably 1 to 3, more preferably 1 or 2), the same or different substituents at any substitutable positions. As the substituents, substituents similar to the above-mentioned Substituent Group V can be mentioned

When W^{b} is C(R^{1b}), compound (Ib) is represented by the following formula (IbA):

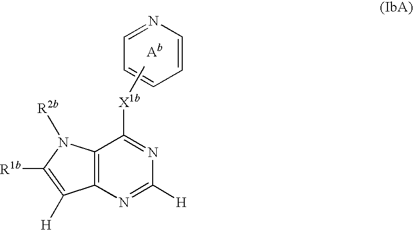

wherein each symbol is as defined above.

When W^{b} is N, compound (Ib) is represented by the following formula (IbB) or (IbC):

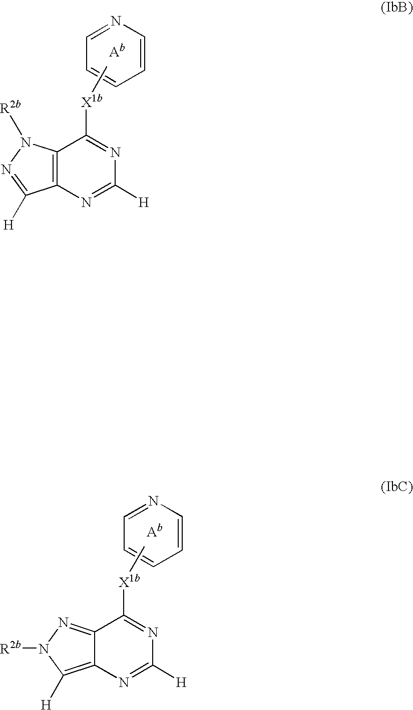

wherein each symbol is as defined above.

In the above-mentioned formulas, the partial structural formula

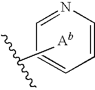

is preferably

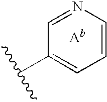

wherein each symbol is as defined above.

As specific examples, a compound represented by the following formula (Ib′) or a salt thereof (in the present specification, hereinafter sometimes to be abbreviated as “compound (Ib′)”) can be mentioned:

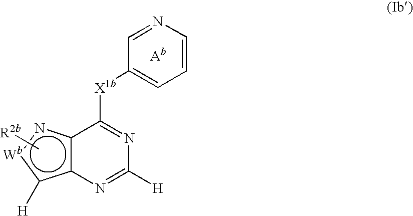

wherein each symbol is as defined above.

### [Compound (Iba)]

As preferable embodiment of compound (Ib), a compound represented by the following formula (Iba) or a salt thereof (in the present specification, hereinafter sometimes to be abbreviated as “compound (Iba)”) can be mentioned:

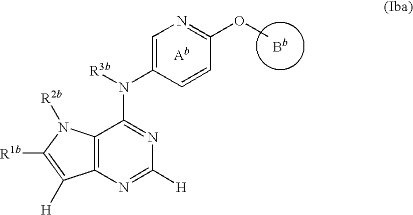

wherein ring A^{b′} is an optionally further substituted pyridine ring, ring B^{b} is an optionally substituted C₆₋₁₄ aryl group, and the other symbols are as defined above.

In the above-mentioned formula (Iba), the “pyridine ring” of the “optionally further substituted pyridine ring” for ring A^{b′} optionally further has, besides the group represented by the formula: —O—B^{b}, 1 to 3, the same or different substituents at any substitutable positions. As the substituents, substituents similar to the above-mentioned Substituent Group V can be mentioned. Of these, halogen and methyl are preferable.

Ring A^{b} is preferably a pyridine ring optionally further substituted, besides the group represented by the formula: —O—B^{b}, by substituent(s) selected from the group consisting of halogen and methyl, more preferably a pyridine ring optionally further substituted, besides the group represented by the formula: —O—B^{b}, by halogen.

The “C₆₋₁₄ aryl group” of the “optionally substituted C₆₋₁₄ aryl group” for ring B^{b} optionally has 1 to 5, the same or different substituents at any substitutable positions. As the substituents, substituents similar to the above-mentioned Substituent Group V can be mentioned. Of these, optionally halogenated C₁₋₆ alkyl, optionally halogenated C₁₋₆ alkoxy, C₁₋₆ alkyl-carbamoyl and halogen are preferable.

Ring B^{b} is preferably a phenyl group optionally substituted by substituent(s) selected from the group consisting of optionally halogenated C₁₋₆ alkyl, optionally halogenated C₁₋₆ alkoxy, C₁₋₆ alkyl-carbamoyl and halogen (preferably a phenyl group optionally substituted by substituent(s) selected from the group consisting of optionally halogenated C₁₋₆ alkyl, optionally halogenated C₁₋₆ alkoxy and C₁₋₆ alkyl-carbamoyl), more preferably a phenyl group optionally substituted at the 3-position by substituent(s) selected from the group consisting of optionally halogenated C₁₋₆ alkyl, optionally halogenated C₁₋₆ alkoxy, C₁₋₆ alkyl-carbamoyl and halogen (preferably, a phenyl group optionally substituted at the 3-position by substituent(s) selected from the group consisting of optionally halogenated C₁₋₆ alkyl, optionally halogenated C₁₋₆ alkoxy and C₁₋₆ alkyl-carbamoyl).

As more preferable embodiment of compound (Ib), compound (Iba) wherein, the above-mentioned formula (Iba),
R^{1b} is a hydrogen atom, a halogen atom, a cyano group or an optionally halogenated C₁₋₆ alkyl group,
R^{2b} is a C₁₋₆ alkyl group substituted by substituent(s) selected from the group consisting of
(i) —NR^{6ba}—CO—(CH₂)ₙ₁—SO₂—C₁₋₄ alkyl
wherein R^{6ba} is a hydrogen atom or a methyl group, n1 is an integer of 1 to 4, and —(CH₂)ₙ₁— is optionally substituted by C₁₋₄ alkyl,
(ii) —NR^{6bb}—CO—(CH₂)ₙ₂—OH
wherein R^{6bb} is a hydrogen atom or a methyl group, n2 is an integer of 1 to 4, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl, and
(iv) hydroxy,
R^{3b} is a hydrogen atom,
ring A^{b′} is a pyridine ring optionally substituted by substituent(s) selected from the group consisting of halogen and methyl, and
ring B^{b} is a phenyl group optionally substituted by substituent(s) selected from the group consisting of optionally halogenated C₁₋₆ alkyl, optionally halogenated C₁₋₆ alkoxy, C₁₋₆ alkyl-carbamoyl and halogen,
can be mentioned.

As another more preferable embodiment of compound (Ib), compound (Iba) wherein, the above-mentioned formula (Iba),
ring A^{b′} is a pyridine ring optionally substituted by halogen, and
ring B^{b} is a phenyl group optionally substituted at the 3-position by substituent(s) selected from the group consisting of optionally halogenated C₁₋₆ alkyl, optionally halogenated C₁₋₆ alkoxy, C₁₋₆ alkyl-carbamoyl and halogen,
can be mentioned.

As compound (Ib), particularly preferably,
2-{2-[4-({5-chloro-6-[3-(trifluoromethyl)phenoxy]pyridin-3-yl}amino)-5H-pyrrolo[3,2-d]pyrimidin-5-yl]ethoxy}ethanol,
N-{2-[4-({5-chloro-6-[3-(trifluoromethyl)phenoxy]pyridin-3-yl}amino)-5H-pyrrolo[3,2-d]pyrimidin-5-yl]ethyl}-2-(methylsulfonyl)acetamide,
N-{2-[4-({5-chloro-6-[3-(trifluoromethyl)phenoxy]pyridin-3-yl}amino)-5H-pyrrolo[3,2-d]pyrimidin-5-yl]ethyl}-3-hydroxy-3-methylbutanamide,
N-{2-[4-({5-chloro-6-[3-(trifluoromethoxy)phenoxy]pyridin-3-yl}amino)-5H-pyrrolo[3,2-d]pyrimidin-5-yl]ethyl}-2-(methylsulfonyl)acetamide, and
N-(tert-butyl)-3-[(3-chloro-5-{[5-(2-hydroxyethyl)-5H-pyrrolo[3,2-d]pyrimidin-4-yl]amino}pyridin-2-yl)oxy]benzamide,
and salts thereof can be mentioned.

### [Compound (Ic)]

The present invention provides also a compound represented by the formula (Ic) or a salt thereof (in the present specification, hereinafter sometimes to be abbreviated as “compound (Ic)”).

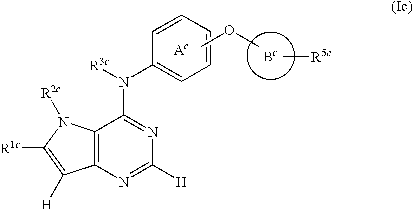

wherein each symbol is as defined above.

In the above-mentioned formula (Ic), as the “optionally substituted group bonded via a carbon atom, a nitrogen atom or an oxygen atom” for R^{1c}, a cyano group and an optionally substituted C₁₋₆ alkyl group are preferable. As the C₁₋₈ alkyl group, a C₁₋₆ alkyl group is preferable.

As the substituents for the alkyl group, substituents similar to the above-mentioned Substituent Group X can be mentioned. Of these, halogen is preferable.

R^{1c} is preferably a hydrogen atom, a cyano group or an optionally halogenated C₁₋₆ alkyl group, more preferably a hydrogen atom or a cyano group, particularly preferably a hydrogen atom.

As the “optionally substituted group bonded via a carbon atom or a sulfur atom” for R^{2c}, an optionally substituted C₁₋₆ alkyl group is preferable. As the C₁₋₈ alkyl group, a C₁₋₆ alkyl group is preferable.

As the substituents for the alkyl group, substituents similar to the above-mentioned Substituent Group X can be mentioned, preferably, substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
can be used, and more preferably, substituent(s) selected from the group consisting of
(i) —NH—CO—CR^{7c}R^{8c}—SO₂—C₁₋₄ alkyl
wherein R^{7c} and R^{8c} are the same or different and each is a hydrogen atom or a C₁₋₄ alkyl group,
(ii) —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
(iii) —O—(CH₂)ₙ₃—OH
wherein n3 is an integer of 1 to 4, —(CH₂)ₙ₃— is optionally substituted by C₁₋₄ alkyl,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
can be used.

As the “aliphatic hydrocarbon group” of the “optionally substituted aliphatic hydrocarbon group” for R^{3c}, a C₁₋₆ alkyl group is preferable.

R^{3c} is preferably a hydrogen atom or a C₁₋₆ alkyl group, more preferably a hydrogen atom.

As the “ring structure” of the “optionally substituted ring structure” formed by R^{3c} bonded to the carbon atom on the adjacent benzene ring, a saturated or unsaturated (preferably saturated) 4 to 8-membered (preferably 5 or 6-membered) nitrogen-containing heterocycle can be mentioned.

Specifically,

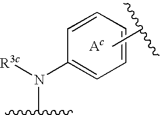

wherein each symbol is as defined above, is, for example,

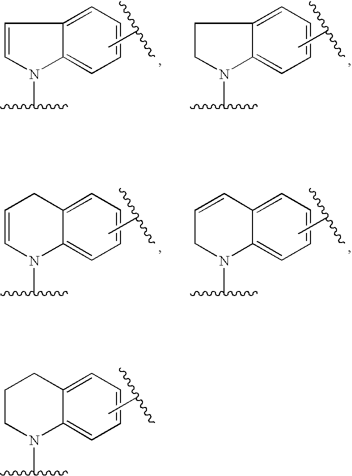

and the like.

The “ring structure” optionally has 1 to 5 (preferably 1 to 3, more preferably 1 or 2), the same or different substituents at any substitutable positions. As the substituents, substituents similar to the above-mentioned Substituent Group V can be mentioned.

R^{1c} and R^{2c} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{1c} and R^{2c} bonded to each other, for example,

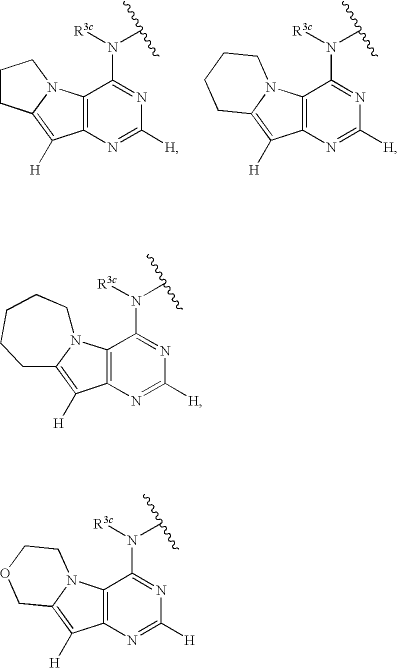

wherein each symbol is as defined above, and the like can be mentioned.

R^{2c} and R^{3c} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure”, formed by R^{2c} and R^{3c} bonded to each other, for example,

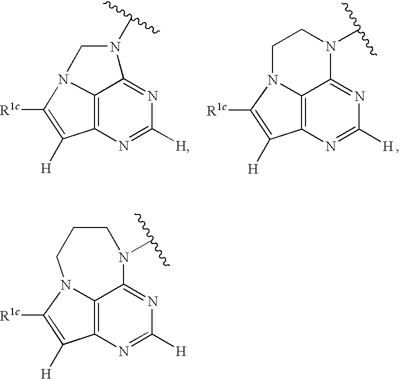

wherein each symbol is as defined above,
and the like can be mentioned.

The “ring structure” of the “optionally substituted ring structure” formed by R^{1c} and R^{2c}, or R^{2c} and R^{3c} optionally has 1 to 5 (preferably 1 to 3, more preferably 1 or 2), the same or different substituents at any substitutable positions. As the substituents, substituents similar to the above-mentioned Substituent Group V can be mentioned.

The “benzene ring” of the “optionally substituted benzene ring” for ring A^{c} optionally has 1 to 3, the same or different substituents at any substitutable positions. As the substituents, substituents similar to the above-mentioned Substituent Group V can be mentioned. Of these, halogen and methyl are preferable.

Ring A^{c} is preferably a benzene ring optionally substituted by substituent(s) selected from the group consisting of halogen and methyl.

As the “optionally substituted amino group” for R^{5c}, an amino group, a mono- or di-C₁₋₆ alkyl-amino group, an optionally halogenated C₁₋₆ alkanoyl-amino group, a hydroxy-C₁₋₆ alkanoyl-amino group, a C₁₋₆ alkanoyl-amino group having hydroxy and halogen, a C₃₋₇ cycloalkyl-C₁₋₆ alkanoyl-amino group, a C₁₋₆ alkanoyl-amino group having C₃₋₇ cycloalkyl and halogen, a C₁₋₆ alkylsulfonyl-C₁₋₆ alkanoyl-amino group, a C₃₋₇ cycloalkyl-carbonyl-amino group and a C₁₋₆ alkoxy-carbonyl-amino group are preferable.

As the “optionally substituted carbamoyl group” for R^{5c}, a carbamoyl group, an optionally halogenated C₁₋₆ alkyl-carbamoyl group, a hydroxy-C₁₋₆ alkyl-carbamoyl group, a C₁₋₆ alkyoxy-C₁₋₆ alkyl-carbamoyl group, a C₆₋₁₄ aryl-C₁₋₆ alkyl-carbamoyl group, a C₂₋₆ alkynyl-carbamoyl group, a piperidyl-C₁₋₆ alkyl-carbamoyl group, a morpholinyl-C₁₋₆ alkyl-carbamoyl group, a C₃₋₇ cycloalkyl-carbamoyl group optionally substituted by C₁₋₆ alkyl or C₂₋₆ alkynyl, and a 5 or 6-membered cyclic amino-carbonyl group optionally containing an oxygen atom are preferable.

As the “optionally substituted ureido group” for R^{5c}, a ureido group, a C₁₋₆ alkyl-ureido group, a C₃₋₇ cycloalkyl-ureido group, and a 5- to 8-membered heterocyclyl-ureido group containing, besides carbon atoms, 1 to 3 hetero atoms selected from the group consisting of a nitrogen atom, an oxygen atom and a sulfur atom are preferable.

As the “optionally substituted sulfamoyl group” for R^{5c}, a sulfamoyl group optionally substituted by C₁₋₆ alkyl is preferable.

As the “optionally substituted heterocyclic group” for R^{5c}, a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl is preferable.

As the “optionally substituted C₂₋₆ alkoxy group” for R^{5c}, a C₂₋₆ alkoxy group optionally substituted by substituent(s) selected from the group consisting of C₃₋₇ cycloalkyl, halogen, C₁₋₆ alkoxy and C₁₋₆ alkyl-carbamoyl is preferable.

As the “optionally substituted aminomethyl group” for R^{5c}, an aminomethyl group optionally substituted by C₁₋₆ alkyl-carbonyl is preferable.

As the “optionally substituted carbamoylmethyl group” for R^{5c}, a carbamoylmethyl group optionally substituted by C₁₋₆ alkyl is preferable.

As the “optionally substituted alkylsulfonyl group” for R^{5c}, a C₁₋₆ alkylsulfonyl group optionally having C₃₋₇ cycloalkyl or halogen is preferable.

As the “C₆₋₁₄ aryl group” of the “optionally further substituted C₆₋₁₄ aryl group” for ring B^{c}, a phenyl group is preferable.

As the “C₅₋₈ cycloalkyl group” of the “optionally further substituted C₅₋₈ cycloalkyl group” for ring B^{c}, a cyclohexyl group is preferable.

The “C₆₋₁₄ aryl group” of the “optionally further substituted C₆₋₁₄ aryl group” for ring B^{c} and the “C₅₋₈ cycloalkyl group” of the “optionally further substituted C₅₋₈ cycloalkyl group” for ring B^{c}, each optionally have, besides R^{5c}, 1 to 5, the same or different substituents at any substitutable positions. As the substituents, substituents similar to the above-mentioned Substituent Group V can be mentioned. Of these, optionally halogenated C₁₋₆ alkyl and halogen are preferable.

In the above-mentioned formula, the partial structural formula

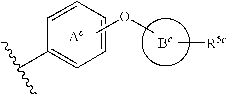

is preferably,

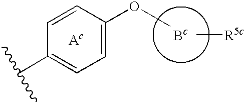

wherein each symbol is as defined above.

As specific examples, a compound represented by the following formula (Ic′) or a salt thereof (in the present specification, hereinafter sometimes to be abbreviated as “compound (Ic′)”), and a compound represented by the following formula (Ic″) or a salt thereof (in the present specification, hereinafter, sometimes to be abbreviated as “compound (Ic″)”) can be mentioned:

### [Compound (Ic′)]

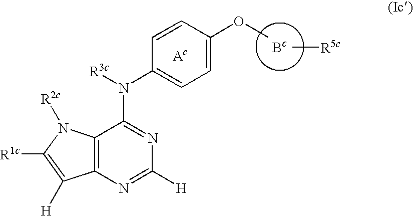 [1149] wherein each symbol is as defined above.

### [Compound (Ic″)]

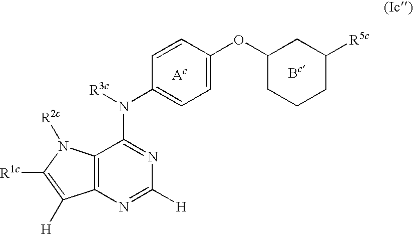 [1151] wherein ring B^{c′} is a phenyl group or a cyclohexyl group, each of which is optionally further substituted besides R^{5c}, and the other symbols are as defined above.

As preferable embodiment of compound (Ic), compound (Ic) wherein
R^{2c} is a C₁₋₆ alkyl group optionally substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
can be mentioned.

As another preferable embodiment of compound (Ic), compound (Ic) wherein
R^{1c} is a hydrogen atom or a cyano group,
R^{2c} is a C₁₋₆ alkyl group optionally substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, ring A^{c} is a benzene ring optionally substituted by substituent(s) selected from the group consisting of halogen and methyl,
R^{5c} is
(i) an amino group,
(ii) a mono-C₁₋₆ alkyl-amino group,
(iii) a di-C₁₋₆ alkyl-amino group,
(iv) an optionally halogenated C₁₋₆ alkanoyl-amino group,
(v) a hydroxy-C₁₋₆ alkanoyl-amino group,
(vi) a C₁₋₆ alkanoyl-amino group having hydroxy and halogen,
(vii) a C₃₋₇ cycloalkyl-C₁₋₆ alkanoyl-amino group,
(viii) a C₁₋₆ alkanoyl-amino group having C₃₋₇ cycloalkyl and halogen,
(ix) a C₁₋₆ alkylsulfonyl-C₁₋₆ alkanoyl-amino group,
(x) a C₃₋₇ cycloalkyl-carbonyl-amino group,
(xi) a C₁₋₆ alkoxy-carbonyl-amino group,
(xii) a carbamoyl group,
(xiii) an optionally halogenated C₁₋₆ alkyl-carbamoyl group,
(xiv) a hydroxy-C₁₋₆ alkyl-carbamoyl group,
(xv) a C₁₋₆ alkoxy-C₁₋₆ alkyl-carbamoyl group,
(xvi) a C₆₋₁₄ aryl-C₁₋₆ alkyl-carbamoyl group,
(xvii) a C₂₋₆ alkynyl-carbamoyl group,
(xviii) a piperidyl-C₁₋₆ alkyl-carbamoyl group,
(xix) a morpholinyl-C₁₋₆ alkyl-carbamoyl group,
(xx) a C₃₋₇ cycloalkyl-carbamoyl group optionally substituted by C₁₋₆ alkyl or C₂₋₆ alkynyl,
(xxi) a 5 or 6-membered cyclic amino-carbonyl group optionally containing an oxygen atom,
(xxii) a ureido group,
(xxiii) a C₁₋₆ alkyl-ureido group,
(xxiv) a C₃₋₇ cycloalkyl-ureido group,
(xxv) a 5- to 8-membered heterocyclyl-ureido group containing, besides carbon atoms, 1 to 3 hetero atoms selected from the group consisting of a nitrogen atom, an oxygen atom and a sulfur atom,
(xxvi) a sulfamoyl group optionally substituted by C₁₋₆ alkyl,
(xxvii) a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl,
(xxviii) a C₂₋₆ alkoxy group optionally substituted by substituent(s) selected from the group consisting of C₃₋₇ cycloalkyl, halogen, C₁₋₆ alkoxy and C₁₋₆ alkyl-carbamoyl,
(xxix) a carbamoylmethyl group optionally substituted by C₁₋₆ alkyl,
(xxx) an aminomethyl group optionally substituted by C₁₋₆ alkyl-carbonyl,
(xxxi) a C₁₋₆ alkylsulfonyl group optionally having C₃₋₇ cycloalkyl or halogen, or
(xxxii) a cyano group, and
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen, can be mentioned.

As another preferable embodiment of compound (Ic), compound (Ic) wherein
R^{5c} is an amino group optionally substituted by substituent(s) selected from the group consisting of
(i) C₁₋₆ alkyl,
(ii) optionally halogenated C₁₋₆ alkanoyl,
(iii) hydroxy-C₁₋₆ alkanoyl,
(iv) C₁₋₆ alkanoyl having hydroxy and halogen,
(v) C₃₋₇ cycloalkyl-C₁₋₆ alkanoyl,
(vi) C₁₋₆ alkanoyl having C₃₋₇ cycloalkyl and halogen,
(vii) C₁₋₆ alkylsulfonyl-C₁₋₆ alkanoyl,
(viii) C₃₋₇ cycloalkyl-carbonyl, and
(ix) C₁₋₆ alkoxy-carbonyl,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and ring A^{c} is a benzene ring optionally substituted by substituent(s) selected from the group consisting of halogen and methyl,
can be mentioned.

In the above-mentioned embodiment, more preferably, R^{2c} is a C₁₋₆ alkyl group substituted by substituent(s) selected from the group consisting of
(i) —NH—CO—CR^{7c}R^{8c}—SO₂—C₁₋₄ alkyl
wherein R^{7c} and R^{8c} are the same or different and each is a hydrogen atom or a C₁₋₄ alkyl group,
(ii) —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino.

As another preferable embodiment of compound (Ic), compound (Ic) wherein
R^{5c} is a carbamoyl group optionally substituted by substituent (s) selected from the group consisting of
(i) optionally halogenated C₁₋₆ alkyl,
(ii) hydroxy-C₁₋₆ alkyl,
(iii) C₁₋₆ alkoxy-C₁₋₆ alkyl,
(iv) C₆₋₁₄ aryl-C₁₋₆ alkyl,
(v) C₂₋₆ alkynyl,
(vi) piperidyl-C₁₋₆ alkyl,
(vii) morpholinyl-C₁₋₆ alkyl, and
(viii) C₃₋₇ cycloalkyl optionally substituted by C₁₋₆ alkyl or C₂₋₆ alkynyl,
ring B^{c} is a C₆₋₁₄ aryl group or a C₅₋₈ cycloalkyl group, each of which is optionally further substituted, besides R^{5c}, by substituent (s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and ring A^{c} is a benzene ring optionally substituted by substituent(s) selected from the group consisting of halogen and methyl,
can be mentioned.

In the above-mentioned embodiment, more preferably, R^{2c} is a C₁₋₆ alkyl group substituted by substituent(s) selected from the group consisting of
(i) —NH—CO—CR^{7c}R^{8c}—SO₂—C₁₋₄ alkyl
wherein R^{7c} and R^{8c} are the same or different and each is a hydrogen atom or a C₁₋₄ alkyl group,
(ii) —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino.

As another preferable embodiment of compound (Ic), compound (Ic) wherein
R^{5c} is a ureido group optionally substituted by substituent(s) selected from the group consisting of
(i) C₁₋₆ alkyl,
(ii) C₃₋₇ cycloalkyl, and
(iii) 5- to 8-membered heterocyclic group containing, besides carbon atoms, 1 to 3 hetero atoms selected from the group consisting of a nitrogen atom, an oxygen atom and a sulfur atom,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and ring A^{c} is a benzene ring optionally substituted by substituent(s) selected from the group consisting of halogen and methyl,
can be mentioned.

In the above-mentioned embodiment, more preferably, R^{2c} is a C₁₋₆ alkyl group substituted by substituent(s) selected from the group consisting of
(i) —NH—CO—CR^{7c}R^{8c}—SO₂—C₁₋₄ alkyl
wherein R^{7c} and R^{8c} are the same or different and each is a hydrogen atom or a C₁₋₄ alkyl group,
(ii) —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino.

As another preferable embodiment of compound (Ic), compound (Ic) wherein
R^{5c} is a sulfamoyl group optionally substituted by C₁₋₆ alkyl,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl, and
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and ring A^{c} is a benzene ring optionally substituted by substituent(s) selected from the group consisting of halogen and methyl,
can be mentioned.

In the above-mentioned embodiment, more preferably, R^{2c} is a C₁₋₆ alkyl group substituted by substituent(s) selected from the group consisting of
(i) —NH—CO—CR^{7c}R^{8c}—SO₂—C₁₋₄ alkyl
wherein R^{7c} and R^{8c} are the same or different and each is a hydrogen atom or a C₁₋₄ alkyl group,
(ii) —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino.

As another preferable embodiment of compound (Ic), compound (Ic) wherein
R^{5c} is a 5- to 8-membered heterocyclic group containing, besides carbon atoms, 1 to 3 hetero atoms selected from the group consisting of a nitrogen, atom, an oxygen atom and a sulfur atom, which is optionally substituted by substituent(s) selected from the group consisting of optionally halogenated C₁₋₆ alkyl and C₁₋₆ alkoxy-carbonyl,
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen,
R^{1c} is a hydrogen atom,
R^{2c} is a C₁₋₆ alkyl group substituted by substituent(s) selected from the group consisting of
(i) —NR^{6c}—CO—(CH₂)ₙ—SO₂-optionally halogenated C₁₋₄ alkyl,
(ii) —NR^{6c}—CO—(CH₂)ₙ—OH,
(iii) —O—(CH₂)ₙ—OH,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group, and ring A^{c} is a benzene ring optionally substituted by substituent(s) selected from the group consisting of halogen and methyl,
can be mentioned.

In the above-mentioned embodiment, more preferably, R^{2c} is a C₁₋₆ alkyl group substituted by substituent(s) selected from the group consisting of
(i) —NH—CO—CR^{7c}R^{8c}—SO₂—C₁₋₄ alkyl
wherein R^{7c} and R^{8c} are the same or different and each is a hydrogen atom or a C₁₋₄ alkyl group,
(ii) —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl,
(iv) hydroxy,
(v) —NR^{6c}—CO—C₁₋₄ alkyl,
(vi) —O—C₁₋₄ alkyl,
(vii) —S—C₁₋₄ alkyl,
(viii) —SO₂—C₁₋₄ alkyl, and
(ix) amino.

As another preferable embodiment of compound (Ic), compound (Ic) wherein
R^{2c} is a C₁₋₆ alkyl group substituted by a group represented by —NR^{6ca}—CO—(CH₂)ₙ₁—SO₂-optionally halogenated C₁₋₄ alkyl
wherein n1 is an integer of 1 to 4, R^{6ca} is a hydrogen atom or a C₁₋₄ alkyl group, and —(CH₂)ₙ₁— is optionally substituted by C₁₋₄ alkyl,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group optionally (a) mono-substituted by C₁₋₆ alkanoyl optionally having C₁₋₆ alkylsulfonyl, or (b) mono- or di-substituted by C₁₋₆ alkyl,
a carbamoyl group optionally substituted by C₁₋₆ alkyl,
(iii) a 5- to 8-membered heterocyclic group containing, besides carbon atoms, 1 to 3 hetero atoms selected from the group consisting of a nitrogen atom, an oxygen atom and a sulfur atom, which is optionally substituted by optionally halogenated C₁₋₆ alkyl,
(iv) a C₂₋₆ alkoxy group optionally substituted by C₃₋₇ cycloalkyl, halogen, C₁₋₆ alkoxy or C₁₋₆ alkyl-carbamoyl,
(v) an aminomethyl group optionally substituted by C₁₋₆ alkyl-carbonyl,
(vi) a C₁₋₆ alkylsulfonyl group optionally substituted by C₃₋₇ cycloalkyl, or
(vii) a cyano group, and
ring B^{c} is a C₆₋₁₄ aryl group optionally further substituted, besides R^{5c}, by substituent(s) selected from the group consisting of optionally halogenated C₁₋₆ alkyl and halogen,
can be mentioned.

In the above-mentioned embodiment, more preferably, R^{2c} is a C₁₋₆ alkyl group substituted by a group represented by —NH—CO—CR^{7c}R^{8c}—SO₂—C₁₋₄ alkyl wherein R^{7c} and R^{8c} are the same or different and each is a hydrogen atom or a C₁₋₄ alkyl group.

As another preferable embodiment of compound (Ic), compound (Ic) wherein
R^{2c} is a C₁₋₆ alkyl group substituted by a group represented by —NR^{6cb}—CO—(CH₂)ₙ₂—OH
wherein n2 is an integer of 1 to 4, R^{6cb} is a hydrogen atom or a C₁₋₄ alkyl group, and —(CH₂)ₙ₂— is optionally substituted by C₁₋₄ alkyl,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group optionally (a) mono-substituted by C₁₋₆ alkanoyl optionally having hydroxy, or (b) mono- or di-substituted by C₁₋₆ alkyl,
(ii) a carbamoyl group optionally substituted by C₁₋₆ alkyl,
(iii) a 5- to 8-membered heterocyclic group containing, besides carbon atoms, 1 to 3 hetero atoms selected from the group consisting of a nitrogen atom, an oxygen atom and a sulfur atom, which is optionally substituted by optionally halogenated C₁₋₆ alkyl,
(iv) a C₂₋₆ alkoxy group optionally substituted by C₃₋₇ cycloalkyl, halogen, C₁₋₆ alkoxy or C₁₋₆ alkyl-carbamoyl,
(v) an aminomethyl group optionally substituted by C₁₋₆ alkyl-carbonyl,
(vi) a C₁₋₆ alkylsulfonyl group optionally substituted by C₃₋₇ cycloalkyl, or
(vii) a cyano group, and
ring B^{c} is a C₆₋₁₄ aryl group optionally further substituted, besides R^{5c}, by substituent(s) selected from the group consisting of optionally halogenated C₁₋₆ alkyl and halogen,
can be mentioned.

In the above-mentioned embodiment, more preferably, R^{2c} is a C₁₋₆ alkyl group substituted by a group represented by —NH—CO—CH₂—CR^{9c}R^{10c}—OH
wherein R^{9c} and R^{10c} are the same or different and each is a C₁₋₄ alkyl group.

As another preferable embodiment of compound (Ic), compound (Ic) wherein
R^{2c} is a C₁₋₆ alkyl group substituted by a group represented by —O—(CH₂)ₙ₃—OH
wherein n3 is an integer of 1 to 4, and —(CH₂)ₙ₃— is optionally substituted by C₁₋₄ alkyl,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group,
(ii) a C₁₋₆ alkyl-amino group,
(iii) an optionally halogenated C₁₋₆ alkanoyl-amino group,
(iv) a hydroxy-C₁₋₆ alkanoyl-amino group,
(v) a C₁₋₆ alkanoyl-amino group having hydroxy and halogen,
(vi) a C₃₋₇ cycloalkyl-C₁₋₆ alkanoyl-amino group,
(vii) a C₁₋₆ alkanoyl-amino group having C₃₋₇ cycloalkyl and halogen,
(viii) a C₃₋₇ cycloalkyl-carbonyl-amino group,
(ix) a C₁₋₆ alkoxy-carbonyl-amino group,
(x) a carbamoyl group,
(xi) an optionally halogenated C₁₋₆ alkyl-carbamoyl group,
(xii) a hydroxy-C₁₋₆ alkyl-carbamoyl group,
(xiii) a C₁₋₆ alkoxy-C₁₋₆ alkyl-carbamoyl group,
(xiv) a C₃₋₇ cycloalkyl-carbamoyl group,
(xv) a ureido group,
(xvi) a C₁₋₆ alkyl-ureido group,
(xvii) a C₃₋₇ cycloalkyl-ureido group,
(xviii) a 5- to 8-membered heterocyclyl-ureido group containing, besides carbon atoms, 1 to 3 hetero atoms selected from the group consisting of a nitrogen atom, an oxygen atom and a sulfur atom,
(xix) a 5 or 6-membered cyclic amino-carbonyl group optionally containing an oxygen atom,
(xx) a 5- to 8-membered heterocyclic group containing, besides carbon atoms, 1 to 3 hetero atoms selected from the group consisting of a nitrogen atom, an oxygen atom and a sulfur atom, which is optionally substituted by optionally halogenated C₁₋₆ alkyl or C₁₋₆ alkoxy-carbonyl,
(xxi) an optionally halogenated C₂₋₆ alkoxy group,
(xxii) a C₁₋₆ alkylsulfonyl group, or
(xxiii) a cyano group, and
ring B^{c} is a C₆₋₁₄ aryl group optionally further substituted, besides R^{5c}, by substituent(s) selected from the group consisting of optionally halogenated C₁₋₆ alkyl and halogen,
can be mentioned.

As another preferable embodiment of compound (Ic), compound (Ic) wherein
R^{2c} is a C₁₋₆ alkyl group substituted by hydroxy,
R^{1c} is a hydrogen atom,
R^{3c} is a hydrogen atom,
ring A^{c} is a benzene ring optionally substituted by substituent(s) selected from the group consisting of halogen and methyl,
R^{5c} is
(i) an amino group optionally (a) mono-substituted by C₁₋₆ alkanoyl optionally having hydroxy, or (b) mono- or di-substituted by C₁₋₆ alkyl,
(ii) a carbamoyl group optionally substituted by optionally halogenated C₁₋₆ alkyl,
(iii) a C₃₋₇ cycloalkyl-carbamoyl group optionally substituted by C₁₋₆ alkyl or C₂₋₆ alkynyl,
(iv) a C₆₋₁₄ aryl-C₁₋₆ alkyl-carbamoyl group,
(v) a hydroxy-C₁₋₆ alkyl-carbamoyl group,
(vi) a morpholinyl-C₁₋₆ alkyl-carbamoyl group,
(vii) a C₂₋₆ alkynyl-carbamoyl group,
(viii) a carbamoylmethyl group optionally substituted by C₁₋₆ alkyl,
(ix) a C₂₋₆ alkoxy group optionally substituted by C₃₋₇ cycloalkyl, halogen, C₁₋₆ alkoxy or C₁₋₆ alkyl-carbamoyl,
(x) an aminomethyl group optionally substituted by C₁₋₆ alkoxy-carbonyl, or
(xi) a C₁₋₆ alkylsulfonyl group optionally substituted by C₃₋₇ cycloalkyl, and
ring B^{c} is a C₆₋₁₄ aryl group optionally further substituted, besides R^{5c}, by substituent(s) selected from the group consisting of optionally halogenated C₁₋₆ alkyl and halogen,
can be mentioned.

As another preferable embodiment of compound (Ic), compound (Ic) wherein
R^{1c} is i a cyano group or an optionally halogenated C₁₋₆ alkyl group,
R^{2c} is
(i) a C₁₋₆ alkyl group, or
(ii) a C₁₋₆ alkyl group substituted by substituent(s) selected from the group consisting of
(a) —NR^{6c}—CO—(CH₂)ₙ—SO₂-optionally halogenated C₁₋₄ alkyl,
(b) —NR^{6c}—CO—(CH₂)ₙ—OH,
(c) —O—(CH₂)ₙ—OH, and
(d) hydroxy
wherein n is an integer of 1 to 4, R^{6c} is a hydrogen atom or a C₁₋₄ alkyl group, and —(CH₂)ₙ— is optionally substituted by C₁₋₄ alkyl,
R^{3c} is a hydrogen atom or a C₁₋₆ alkyl group,
ring A^{c} is a benzene ring optionally substituted by substituent(s) selected from the group consisting of halogen and methyl,
R^{5c} is
(i) an amino group,
(ii) a C₁₋₆ alkyl-amino group,
(iii) an optionally halogenated C₁₋₆ alkanoyl-amino group,
(iv) a hydroxy-C₁₋₆ alkanoyl-amino group,
(v) a C₁₋₆ alkanoyl-amino group having hydroxy and halogen,
(vi) a C₃₋₇ cycloalkyl-C₁₋₆ alkanoyl-amino group,
(vii) a C₁₋₆ alkanoyl-amino group having C₃₋₇ cycloalkyl and halogen,
(viii) a C₁₋₆ alkylsulfonyl-C₁₋₆ alkanoyl-amino group,
(ix) a C₃₋₇ cycloalkyl-carbonyl-amino group,
(x) a C₁₋₆ alkoxy-carbonyl-amino group,
(xi) a carbamoyl group,
(xii) an optionally halogenated C₁₋₆ alkyl-carbamoyl group,
(xiii) a hydroxy-C₁₋₆ alkyl-carbamoyl group,
(xiv) a C₁₋₆ alkoxy-C₁₋₆ alkyl-carbamoyl group,
(xv) a C₃₋₇ cycloalkyl-carbamoyl group,
(xvi) a 5 or 6-membered cyclic amino-carbonyl group optionally containing an oxygen atom,
(xvii) a ureido group,
(xviii) a C₁₋₆ alkyl-ureido group,
(xix) a C₃₋₇ cycloalkyl-ureido group,
(xx) a 5- to 8-membered heterocyclyl-ureido group containing, besides carbon atoms, 1 to 3 hetero atoms selected from the group consisting of a nitrogen atom, an oxygen atom and a sulfur atom,
(xxi) a sulfamoyl group optionally substituted by C₁₋₆ alkyl, or
(xxii) a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl, and
ring B^{c} is a C₆₋₁₄ aryl group or a C₅₋₈ cycloalkyl group, each of which is optionally further substituted, besides R^{5c}, by substituent(s) selected from the group consisting of optionally halogenated C₁₋₆ alkyl and halogen, can be mentioned.

Of the above-mentioned preferable embodiments of compound (Ic), a compound corresponding compound (Ic″) is particularly preferable. That is,
(i) a compound wherein ring B^{c} is a phenyl group or a cyclohexyl group, each of which is optionally further substituted, besides R^{5c}, by substituent(s) selected from the group consisting of optionally halogenated C₁₋₆ alkyl and halogen, and is substituted by R^{5c} at the meta-position of the phenyl group or the p-position of the cyclohexyl group, and
(ii) a compound wherein ring B^{c} is a phenyl group optionally further substituted, besides R^{5c}, by substituent(s) selected from the group consisting of optionally halogenated C₁₋₆ alkyl and halogen, which phenyl is substituted by R^{5c} at the meta-position of the phenyl group,
are particularly preferable.

As compound (Ic), particularly preferably,
2-{2-[4-({3-chloro-4-[3-(1,3-thiazol-5-yl)phenoxy]phenyl}amino)-5H-pyrrolo[3,2-d]pyrimidin-5-yl]ethoxy}ethanol,
N-(tert-butyl)-3-[2-chloro-4-({5-[2-(2-hydroxyethoxy)ethyl]-5H-pyrrolo[3,2-d]pyrimidin-4-yl}amino)phenoxy]benzamide,
3-[2-chloro-4-({5-[2-(2-hydroxyethoxy)ethyl]-5H-pyrrolo[3,2-d]pyrimidin-4-yl}amino)phenoxy]-N-(2-hydroxy-1,1-dimethylethyl)benzamide,
N-(tert-butyl)-3-(2-chloro-4-{[5-(2-hydroxyethyl)-5H-pyrrolo[3,2-d]pyrimidin-4-yl]amino}phenoxy)benzamide,
N-(3-{2-chloro-4-[(6-cyano-5-methyl-5H-pyrrolo[3,2-d]pyrimidin-4-yl)amino]phenoxy}phenyl)cyclopropanecarboxamide,
N-(tert-butyl)-5-(2-chloro-4-{[5-(2-hydroxyethyl)-5H-pyrrolo[3,2-d]pyrimidin-4-yl]amino}phenoxy)-2-fluorobenzamide,
N-{2-[4-({3-chloro-4-[3-(dimethylamino)phenoxy]phenyl}amino)-5H-pyrrolo[3,2-d]pyrimidin-5-yl]ethyl}-3-hydroxy-3-methylbutanamide,
N-{2-[4-({3-chloro-4-[3-(dimethylamino)phenoxy]phenyl}amino)-5H-pyrrolo[3,2-d]pyrimidin-5-yl]ethyl}-2-(methylsulfonyl)acetamide,
N-(tert-butyl)-2-[3-(2-chloro-4-{[5-(2-hydroxyethyl)-5H-pyrrolo[3,2-d]pyrimidin-4-yl]amino}phenoxy)phenyl]acetamide,
N-{2-[4-({3-chloro-4-[3-(cyclopropylmethoxy)phenoxy]phenyl}amino)-5H-pyrrolo[3,2-d]pyrimidin-5-yl]ethyl}-2-(methylsulfonyl)acetamide,
N-{2-[4-({3-chloro-4-[3-(2,2-dimethylpropoxy)phenoxy]phenyl}amino)-5H-pyrrolo[3,2-d]pyrimidin-5-yl]ethyl}-2-(methylsulfonyl)acetamide,
2-(methylsulfonyl)-N-{2-[4-({3-methyl-4-[3-(2,2,2-trifluoroethoxy)phenoxy]phenyl}amino)-5H-pyrrolo[3,2-d]pyrimidin-5-yl]ethyl}acetamide,
2-[4-({3-chloro-4-[3-(isopropylsulfonyl)phenoxy]phenyl}amino)-5H-pyrrolo[3,2-d]pyrimidin-5-yl]ethanol, and
N-[2-(4-{[3-chloro-4-(3-cyanophenoxy)phenyl]amino}-5H-pyrrolo[3,2-d]pyrimidin-5-yl)ethyl]-2-(methylsulfonyl)acetamide,
and salts thereof can be mentioned.

### [Compound (Id)]

The present invention-also provides a compound represented by the formula (Id) or a salt thereof (in the present specification, hereinafter sometimes to be abbreviated as “compound (Id)”).

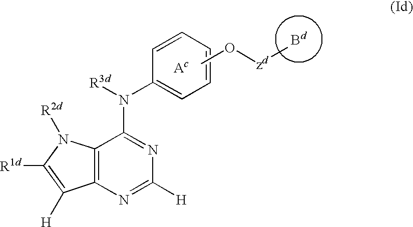

wherein each symbol is as defined above.

In the above-mentioned formula (Id), as the “optionally substituted group bonded via a carbon atom, a nitrogen atom or an oxygen atom” for R^{1d}, a cyano group and an optionally substituted C₁₋₈ alkyl group are preferable. As the C₁₋₈ alkyl group, a C₁₋₆ alkyl group is preferable.

As the substituents for the alkyl group, substituents similar to the above-mentioned Substituent Group X can be mentioned. Of these, halogen is preferable.

R^{1d} is preferably a hydrogen atom, a cyano group or an optionally halogenated C₁₋₆ alkyl group, more preferably a hydrogen atom.

As the “optionally substituted group bonded via a carbon atom or a sulfur atom” for R^{2d}, an optionally substituted C₁₋₈ alkyl group is preferable. As the C₁₋₈ alkyl group, a C₁₋₆ alkyl group is preferable.

As the substituents for the alkyl group, substituents similar to the above-mentioned Substituent Group X can be mentioned, preferably, substituent(s) selected from the group consisting of
(a) —NR^{6d}—CO—(CH₂)ₙ—SO₂-optionally halogenated C₁₋₄ alkyl,
(b) —NR^{6d}—CO—(CH₂)ₙ—OH,
(c) —O—(CH₂)ₙ—OH, and
(d) hydroxy
wherein n is an integer of 1 to 4, R^{6d} is a hydrogen atom or a C₁₋₄ alkyl group, and —(CH₂)ₙ— is optionally substituted by C₁₋₄ alkyl,
can be used.
As the “aliphatic hydrocarbon group” of the “optionally substituted aliphatic hydrocarbon group” for R^{3d}, a C₁₋₆ alkyl group is preferable.

R^{3d} is preferably a hydrogen atom or a C₁₋₆ alkyl group, more preferably a hydrogen atom.

As the “ring structure” of the “optionally substituted ring structure” formed by R^{3d} bonded to the carbon atom on the adjacent benzene ring, a saturated or unsaturated (preferably saturated) 4 to 8-membered (preferably 5 or 6-membered) nitrogen-containing heterocycle can be mentioned.

Specifically,

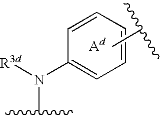

wherein each symbol is as defined above, is, for example,

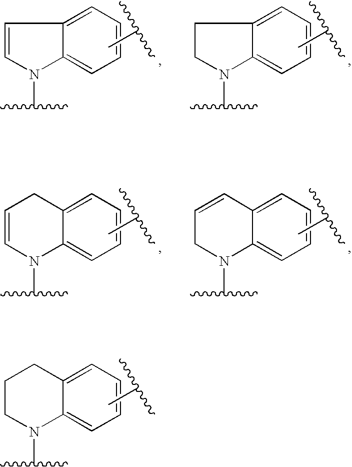

and the like.

The “ring structure” optionally has 1 to 5 (preferably 1 to 3, more preferably 1 or 2), the same or different substituents at any substitutable positions. As the substituents, substituents similar to the above-mentioned Substituent Group V can be mentioned.

R^{1d} and R^{2d} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{1d} and R^{2d} bonded to each other, for example,

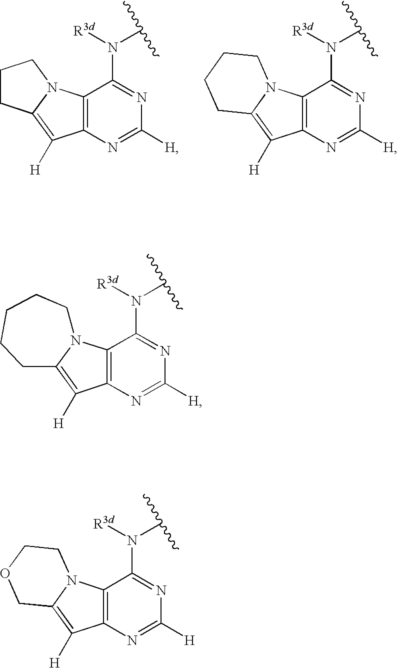

wherein each symbol is as defined above,
and the like can be mentioned.

R^{2d} and R^{3d} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{2d} and R^{3d} bonded to each other, for example,

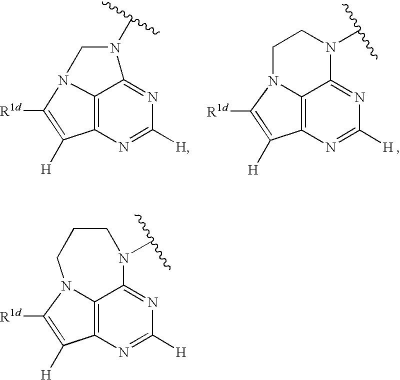

wherein each symbol is as defined above,
and the like can be mentioned.

The “ring structure” of the “optionally substituted ring structure” formed by R^{1d} and R^{2d}, or R^{2d} and R^{3d} optionally has 1 to 5 (preferably 1 to 3, more preferably 1 or 2), the same or different substituents at any substitutable positions. As the substituents, substituents similar to the above-mentioned Substituent Group V can be mentioned

The “benzene ring” of the “optionally substituted benzene ring” for ring A^{d} optionally has 1 to 3, the same or different substituents at any substitutable positions. As the substituents, substituents similar to the above-mentioned Substituent Group V can be mentioned. Of these, halogen and methyl are preferable.

Ring A^{d} is preferably a benzene ring optionally substituted by substituent(s) selected from the group consisting of halogen and methyl, more preferably a benzene ring optionally substituted by halogen.

As the “heterocyclic group” of the “optionally substituted heterocyclic group” for ring B^{d}, a 5 or 6-membered monocyclic heterocyclic group is preferable, and a piperidyl group is more preferable.

The “heterocyclic group” of the “optionally substituted heterocyclic group” for ring B^{d} optionally has 1 to 5, the same or different substituents at any substitutable positions. As the substituents, acyl and substituents similar to the above-mentioned Substituent Group V can be mentioned. Of these, acyl and optionally substituted ureido are preferable, and C₁₋₆ alkoxy-carbonyl, C₅₋₈ cycloalkyl-carbonyl, C₁₋₆ alkyl-ureido and C₅₋₈ cycloalkyl-ureido are more preferable.

Ring B^{d} is preferably a heterocyclic group (preferably, a 5 or 6-membered monocyclic heterocyclic group, more preferably, a piperidyl group) optionally substituted by acyl or optionally substituted ureido, more preferably a heterocyclic group (preferably, a 5 or 6-membered monocyclic heterocyclic group, more preferably, a piperidyl group) optionally substituted by C₁₋₆ alkoxy-carbonyl, C₅₋₈ cycloalkyl-carbonyl, C₁₋₆ alkyl-ureido or C₅₋₈ cycloalkyl-ureido.

As the “C₁₋₃ alkylene” of the “optionally substituted C₁₋₃ alkylene” for Z^{d}, methylene is preferable.

The “C₁₋₃ alkylene” of the “optionally substituted C₁₋₃ alkylene” for Z^{d} is optionally substituted by 1 to 3 substituents selected from the group consisting of halogen, hydroxy, C₁₋₄ alkoxy, C₁₋₄ alkyl-carbonyl, carboxy, C₁₋₄ alkoxy-carbonyl, cyano, carbamoyl, sulfamoyl, nitro, amino, C₁₋₄ alkyl-carbonylamino, C₁₋₄ alkoxy-carbonylamino and C₁₋₄ alkylsulfonylamino.

In the above-mentioned formula, the partial structural formula

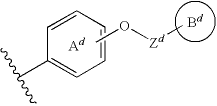

is preferably

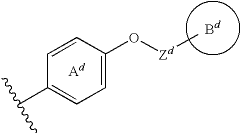

wherein each symbol is as defined above.

As specific examples, a compound represented by the following formula (Id′) or a salt thereof (in the present specification, hereinafter sometimes to be abbreviated as “compound (Id′)”) can be mentioned:

### [Compound (Id′)]

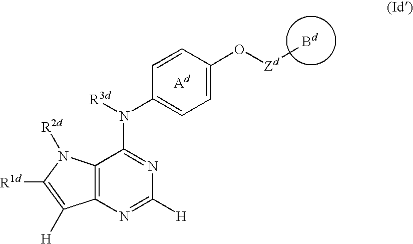 [1566] wherein each symbol is as defined above.

As preferable embodiment of compound (Id), a compound represented by the following formula (Ida) or a salt thereof (in the present specification, hereinafter sometimes to be abbreviated as “compound (Ida)”) can be mentioned:

### [Compound (Ida)]

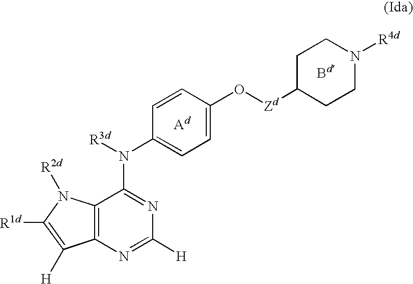 [1569] wherein R^{4d} is an acyl group or an optionally substituted ureido group, ring B^{d′} is a piperidyl group optionally further substituted besides R^{4d}, and the other symbols are as defined above.

In the above-mentioned formula (Ida), as the “acyl group” for R^{4d}, a C₁₋₆ alkoxy-carbonyl group and a C₅₋₈ cycloalkyl-carbonyl group are preferable.

In the above-mentioned formula (Ida), as the “optionally substituted ureido group” for R^{4d}, a C₁₋₆ alkyl-ureido group and a C₅₋₈ cycloalkyl-ureido group are preferable.

The “piperidyl group” of the “optionally further substituted piperidyl group” for ring B^{d′} optionally has, besides R^{4d}, 1 to 5, the same or different substituents at any substitutable positions. As the substituents, substituents similar to the above-mentioned Substituent Group V can be mentioned.

As more preferable embodiment of compound (Id), compound (Ida) wherein, in the above-mentioned formula (Ida),
R^{1d} is a hydrogen atom, a cyano group or an optionally halogenated C₁₋₆ alkyl group,
R^{2d} is
(i) a C₁₋₆ alkyl group, or
(ii) a C₁₋₆ alkyl group substituted by substituent(s) selected from the group consisting of
(a) —NR^{6d}—CO—(CH₂)ₙ—SO₂-optionally halogenated C₁₋₄ alkyl,
(b) —NR^{6d}—CO—(CH₂)ₙ—OH,
(c) —O—(CH₂)ₙ—OH, and
(d) hydroxy
wherein n is an integer of 1 to 4, R^{6d} is a hydrogen atom or a C₁₋₄ alkyl group, and —(CH₂)ₙ— is optionally substituted by C₁₋₄ alkyl,
R^{3d} is a hydrogen atom or a C₁₋₆ alkyl group,
ring A^{d} is a benzene ring optionally substituted by substituent(s) selected from the group consisting of halogen and methyl,
Z^{d} is methylene,
ring B^{d′} is a piperidyl group, and
R^{4d} is a C₁₋₆ alkoxy-carbonyl group, a C₅₋₈ cycloalkyl-carbonyl group, a C₁₋₆ alkyl-ureido group or a C₅₋₈ cycloalkyl-ureido group,
can be mentioned.

As another more preferable embodiment of compound (Id), compound (Ida) wherein, the above-mentioned formula (Ida),
R^{3d} is a hydrogen atom, and
ring A^{d} is a benzene ring optionally substituted by substituent(s) selected from the group consisting of halogen and methyl,
can be mentioned.

As compound (Id), particularly preferably, tert-butyl 4-{[2-chloro-4-({5-[2-(2-hydroxyethoxy)ethyl]-5H-pyrrolo[3,2-d]pyrimidin-4-yl}amino)phenoxy]methyl}piperidine-1-carboxylate, and tert-butyl 4-[(2-chloro-4-{[5-(2-hydroxyethyl)-5H-pyrrolo[3,2-d]pyrimidin-4-yl]amino}phenoxy)methyl]piperidine-1-carboxylate, and salts thereof can be mentioned.

### [Compound (Ie)]

The present invention provides also a compound represented by the formula (Ie) or a salt thereof (in the present specification, hereinafter sometimes to be abbreviated as “compound (Ie)”).

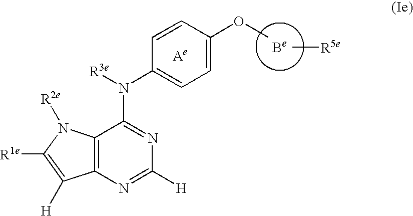

wherein each symbol is as defined above.

In the above-mentioned formula (Ie), as the “optionally substituted group bonded via a carbon atom, a nitrogen atom or an oxygen atom” for R^{1e}, a cyano group and an optionally substituted C₁₋₈ alkyl group are preferable. As the C₁₋₈ alkyl group, a C₁₋₆ alkyl group is preferable.

As the substituents for the alkyl group, substituents similar to the above-mentioned Substituent Group X can be mentioned. Of these, halogen is preferable.

R^{1e} is preferably a hydrogen atom, a cyano group or an optionally halogenated C₁₋₆ alkyl group, more preferably a hydrogen atom or a cyano group, particularly preferably a hydrogen atom.

As the “optionally substituted group bonded via a carbon atom or a sulfur atom” for R^{2e}, an optionally substituted C₁₋₈ alkyl group is preferable. As the C₁₋₈ alkyl group, a C₁₋₆ alkyl group is preferable.

As the substituents for the alkyl group, substituents similar to the above-mentioned Substituent Group X can be mentioned, preferably, substituent(s) selected from the group consisting of
(a) —NR^{6e}—CO—(CH₂)ₙ—SO₂-optionally halogenated C₁₋₄ alkyl,
(b) —NR^{6e}—CO—(CH₂)ₙ—OH,
(c) —O—(CH₂)ₙ—OH, and
(d) hydroxy
wherein n is an integer of 1 to 4, R^{6e} is a hydrogen atom or a C₁₋₄ alkyl group, and —(CH₂)ₙ— is optionally substituted by C₁₋₄ alkyl,
can be used.

As the “aliphatic hydrocarbon group” of the “optionally substituted aliphatic hydrocarbon group” for R^{3e}, a C₁₋₆ alkyl group is preferable.

R^{3e} is preferably a hydrogen atom or a C₁₋₆ alkyl group, more preferably a hydrogen atom.

As the “ring structure” of the “optionally substituted ring structure” formed by R^{3e} bonded to the carbon atom on the adjacent benzene ring, a saturated or unsaturated (preferably saturated) 4 to 8-membered (preferably 5 or 6-membered) nitrogen-containing heterocycle can be mentioned.

Specifically,

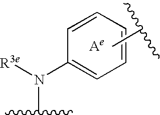

wherein each symbol is as defined above, is, for example,

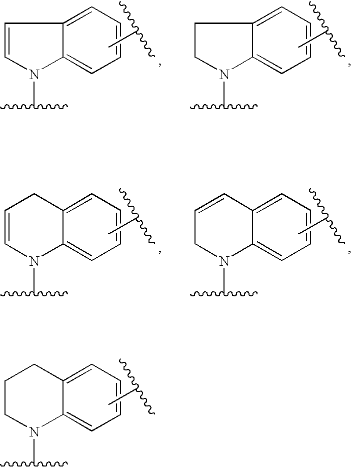

and the like.

The “ring structure” optionally has 1 to 5 (preferably 1 to 3, more preferably 1 or 2), the same or different substituents at any substitutable positions. As the substituents, substituents similar to the above-mentioned Substituent Group V can be mentioned.

R^{1e} and R^{2e} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{1e} and R^{2e} bonded to each other, for example,

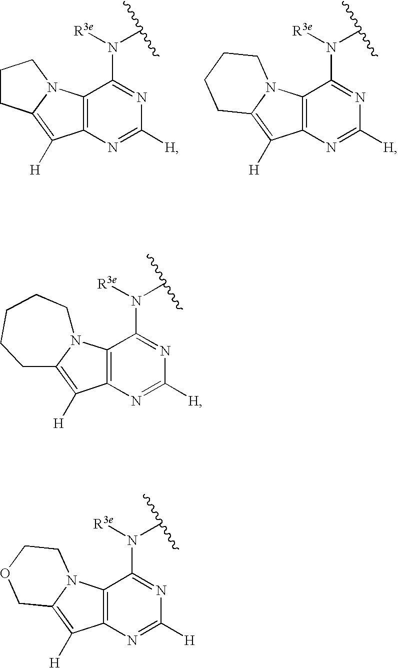

wherein each symbol is as defined above,
and the like can be mentioned.

R^{2e} and R^{3e} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{2e} _{and R}^{3e} _{bonded} to each other, for example,

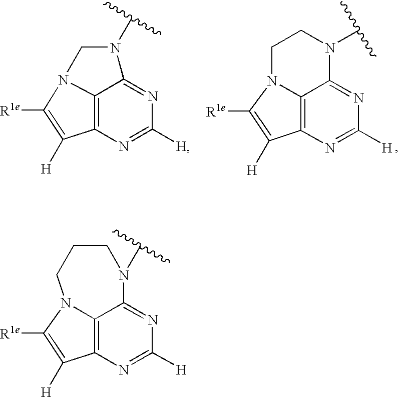

wherein each symbol is as defined above,
and the like can be mentioned.

The “ring structure” of the “optionally substituted ring structure” formed by R^{1e} and R^{2e}, or R^{2e} and R^{3e} optionally has 1 to 5 (preferably 1 to 3, more preferably 1 or 2), the same or different substituents at any substitutable positions. As the substituents, substituents similar to the above-mentioned Substituent Group V can be mentioned

The “benzene ring” of the “optionally substituted benzene ring” for ring A^{e} optionally has 1 to 3, the same or different substituents at any substitutable positions. As the substituents, substituents similar to the above-mentioned Substituent Group V can be mentioned. Of these, halogen and methyl are preferable.

Ring A^{e} is preferably a benzene ring optionally substituted by substituent(s) selected from the group consisting of halogen and methyl.

As the linear alkyl group at R^{5e}, a linear alkyl group having 1 to 10 (preferably 1 to 8, more preferably 1 to 6) carbon atoms can be mentioned. Specifically, methyl, ethyl, propyl, butyl, pentyl, hexyl, heptyl, octyl, nonyl and decyl can be mentioned.

As the branched alkyl group at R^{5e}, a branched alkyl group having 3 to 10 (preferably 3 to 8, more preferably 3 to 6) carbon atoms can be mentioned. Specifically, isopropyl, isobutyl, sec-butyl, tert-butyl, isopentyl, neopentyl, 1-ethylpropyl, isohexyl, 1,1-dimethylbutyl, 2,2-dimethylbutyl, 3,3-dimethylbutyl, 2-ethylbutyl and the like can be mentioned.

As the substituent for the “aryl” of the “linear alkyl group substituted by optionally substituted aryl, which is optionally further halogenated or hydroxylated”, “hydroxy group substituted by optionally substituted aryl” and “C₁₋₆ alkyl-carbonyl group optionally substituted by optionally substituted aryl” for R^{5e}, substituents similar to the above-mentioned Substituent Group V can be mentioned.

As the substituent of “optionally substituted branched alkyl group”, “optionally substituted alkenyl group” and “optionally substituted cycloalkyl group” for R^{5e}, substituents similar to the above-mentioned Substituent Group U can be mentioned.

As the “linear alkyl group substituted by optionally substituted heterocyclic group” for R^{5e}, a 5- to 8-membered heterocyclyl-linear C₁₋₆ alkyl group containing, besides carbon atoms, 1 to 3 hetero atoms selected from the group consisting of a nitrogen atom, an oxygen atom and a sulfur atom, and optionally having C₁₋₆ alkyl is preferable.

As the “linear alkyl group substituted by optionally substituted imino” for R^{5e}, a linear C₁₋₆ alkyl group substituted by hydroxyimino or C₁₋₆ alkoxyimino is preferable.

As the “linear alkyl group substituted by optionally substituted aryl, which is optionally further halogenated or hydroxylated” for R^{5e}, a linear C₁₋₆ alkyl group substituted by C₆₋₁₄ aryl, which is optionally further halogenated or hydroxylated is preferable.

As the “optionally substituted branched alkyl group” for R^{5e}, an optionally halogenated branched C₃₋₆ alkyl group is preferable.

As the “optionally substituted alkenyl group” for R^{5e}, a C₂₋₆ alkenyl group is preferable.

As the “hydroxy group substituted by optionally substituted aryl” for R^{5e}, a hydroxy group substituted by C₆₋₁₄ aryl is preferable.

As the “halogenated C₂₋₆ alkyl” of the “hydroxy group substituted by halogenated C₂₋₆ alkyl” for R^{5e} and “halogenated C₂₋₆ alkyl group” for R^{5e}, ethyl, propyl, isopropyl, butyl, isobutyl, t-butyl, pentyl, isopentyl, neopentyl, hexyl and the like, each of which is halogenated, can be mentioned. Of these, halogenated ethyl is preferable.

As the “optionally substituted cycloalkyl group” for R^{5e}, a C₃₋₇ cycloalkyl group optionally substituted by cyano or carbamoyl is preferable.

As the “C₁₋₆ alkyl-carbonyl group optionally substituted by optionally substituted aryl” for R^{5e}, C₁₋₆ alkyl-carbonyl group optionally substituted by phenyl is preferable.

As the “linear alkyl group substituted by optionally substituted heterocyclic group” for R^{5e},
(i) a methyl group substituted by optionally substituted heterocyclic group, and
(ii) a linear alkyl group substituted by substituted heterocyclic group
are preferable.

The “C₆₋₁₄ aryl group” of the “optionally further substituted C₆₋₁₄ aryl group” for ring B^{e} optionally has, besides R^{5c}, 1 to 3, the same or different substituents at any substitutable positions. As the substituents, substituents similar to the above-mentioned Substituent Group V can be mentioned. Of these, optionally halogenated C₁₋₆ alkyl and halogen are preferable.

Ring B^{e} is preferably a C₆₋₁₄ aryl group (preferably, a phenyl group) optionally further substituted, besides R^{5e}, by substituent(s) selected from the group consisting of optionally halogenated C₁₋₆ alkyl and halogen.

In the above-mentioned formula, the partial structural formula

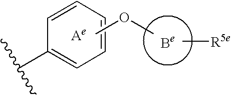

is preferably

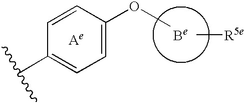

wherein each symbol is as defined above.

As specific examples, a compound represented by the following formula (Ie′) or a salt thereof (in the present specification, hereinafter sometimes to be abbreviated as “compound (Ie′)”) can be mentioned:

### [Compound (Ie′)]

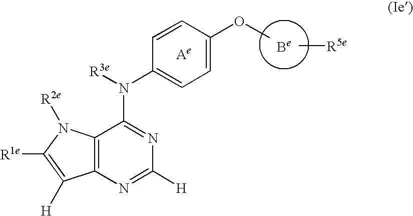 [1649] wherein each symbol is as defined above.

As preferable embodiment of compound (Ie), compound (Ie) wherein
R^{1e} is a hydrogen atom, a cyano group or an optionally halogenated C₁₋₆ alkyl group,
R^{2e} is
(i) a C₁₋₆ alkyl group, or
(ii) a C₁₋₆ alkyl group substituted by substituent(s) selected from the group consisting of
(a) —NR^{6e}—CO—(CH₂)ₙ—SO₂-optionally halogenated C₁₋₄ alkyl,
(b) —NR^{6e}—CO—(CH₂)ₙ—OH,
(c) —O—(CH₂)ₙ—OH, and
(d) hydroxy
wherein n is an integer of 1 to 4, R^{6e} is a hydrogen atom or a C₁₋₄ alkyl group, and —(CH₂)ₙ— is optionally substituted by C₁₋₄ alkyl,
R^{3e} is a hydrogen atom,
ring A^{e} is a benzene ring optionally substituted by substituent(s) selected from the group consisting of halogen and methyl,
R^{5e} is
(i) a 5- to 8-membered heterocyclyl-linear C₁₋₆ alkyl group containing, besides carbon atoms, 1 to 3 hetero atoms selected from the group consisting of a nitrogen atom, an oxygen atom and a sulfur atom, and optionally having C₁₋₆ alkyl,
(ii) a linear C₁₋₆ alkyl group substituted by hydroxyimino or C₁₋₆ alkoxyimino,
(iii) a linear C₁₋₆ alkyl group substituted by C₆₋₁₄ aryl, which is optionally further halogenated or hydroxylated,
(iv) an optionally halogenated branched C₃₋₆ alkyl group,
(v) a C₂₋₆ alkenyl group,
(vi) a hydroxy group substituted by C₆₋₁₄ aryl,
(vii) a hydroxy group substituted by C₁₋₆ alkyl,
(viii) a hydroxy group substituted by halogenated C₂₋₆ alkyl,
(ix) a halogenated C₂₋₆ alkyl group,
(x) a C₃₋₇ cycloalkyl group optionally substituted by cyano or carbamoyl, or
(xi) a C₁₋₆ alkyl-carbonyl group optionally substituted by phenyl, and
ring B^{e} is a C₆₋₁₄ aryl group optionally further substituted, besides R^{5e}, by substituent(s) selected from the group consisting of optionally halogenated C₁₋₆ alkyl and halogen,
can be mentioned.

In the above-mentioned preferable embodiment of compound (Ie), a compound wherein
the “5- to 8-membered heterocyclyl-linear C₁₋₆ alkyl group containing, besides carbon atoms, 1 to 3 hetero atoms selected from the group consisting of a nitrogen atom, an oxygen atom and a sulfur atom, and optionally having C₁₋₆ alkyl” for R^{5e} is
(i) a 5- to 8-membered heterocyclyl-methyl group containing, besides carbon atoms, 1 to 3 hetero atoms selected from the group consisting of a nitrogen atom, an oxygen atom and a sulfur atom, and optionally having C₁₋₆ alkyl, or
(ii) a 5- to 8-membered heterocyclyl-linear C₁₋₆ alkyl group containing, besides carbon atoms, 1 to 3 hetero atoms selected from the group consisting of a nitrogen atom, an oxygen atom and a sulfur atom, and having C₁₋₆ alkyl,
is preferable.

As compound (Ie), particularly preferably,
2-[4-({3-chloro-4-[3-(1,1-difluoroethyl)phenoxy]phenyl}amino)-5H-pyrrolo[3,2-d]pyrimidin-5-yl]ethanol,
(1Z)-1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one O-ethyloxime,
1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-ol,
1-[3-(2-chloro-4-{[5-(2-hydroxyethyl)-5H-pyrrolo[3,2-d]pyrimidin-4-yl]amino}phenoxy)phenyl]-3,3-dimethylbutan-1-one,
N-(2-{4-[(3-methyl-4-{3-[(1E)-3-methylbut-1-en-1-yl]phenoxy}phenyl)amino]-5H-pyrrolo[3,2-d]pyrimidin-5-yl}ethyl)-2-(methylsulfonyl)acetamide, and
N-{2-[4-({3-chloro-4-[3-(1-cyanocyclopropyl)phenoxy]phenyl}amino)-5H-pyrrolo[3,2-d]pyrimidin-5-yl]ethyl}-2-(methylsulfonyl)acetamide,
and salts thereof can be mentioned.

### [Compound (If)]

The present invention provides also a compound represented by the formula (If) or a salt thereof (in the present specification, hereinafter sometimes to be abbreviated as “compound (If)”).

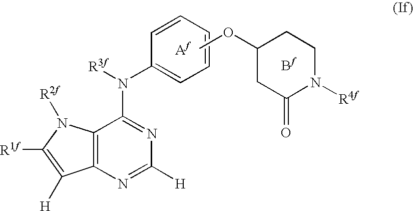

wherein each symbol is as defined above.

In the above-mentioned formula (If), as the “optionally substituted group bonded via a carbon atom, a nitrogen atom or an oxygen atom” for R^{1f}, a cyano group and an optionally substituted C₁₋₈ alkyl group are preferable. As the C₁₋₈ alkyl group, a C₁₋₆ alkyl group is preferable.

As the substituents for the alkyl group, substituents similar to the above-mentioned Substituent Group X can be mentioned. Of these, halogen is preferable.

R^{1f} is preferably a hydrogen atom, a cyano group or an optionally halogenated C₁₋₆ alkyl group, more preferably a hydrogen atom or a cyano group, particularly preferably a hydrogen atom.

As the “optionally substituted group bonded via a carbon atom or a sulfur atom” for R^{2f}, an optionally. substituted C₁₋₈ alkyl group is preferable. As the C₁₋₈ alkyl group, a C₁₋₆ alkyl group is preferable.

As the substituents for the alkyl group, substituents similar to the above-mentioned Substituent Group X can be mentioned, preferably, substituent(s) selected from the group consisting of
(a) —NR^{6f}—CO—(CH₂)ₙ—SO₂-optionally halogenated C₁₋₄ alkyl,
(b) —NR^{6f}—CO—(CH₂)ₙ—OH,
(c) —O—(CH₂)ₙ—OH, and
(d) hydroxy
wherein n is an integer of 1 to 4, R^{6f} is a hydrogen atom or a C₁₋₄ alkyl group, and —(CH₂)ₙ— is optionally substituted by C₁₋₄ alkyl,
can be used.

As the “aliphatic hydrocarbon group” of the “optionally substituted aliphatic hydrocarbon group” for R^{3f}, a C₁₋₆ alkyl group is preferable.

R^{3f} is preferably a hydrogen atom or a C₁₋₆ alkyl group, more preferably a hydrogen atom.

As the “ring structure” of the “optionally substituted ring structure” formed by R^{3f} bonded to the carbon atom on the adjacent benzene ring, a saturated or unsaturated (preferably saturated) 4 to 8-membered (preferably 5 or 6-membered) nitrogen-containing heterocycle can be mentioned.

Specifically,

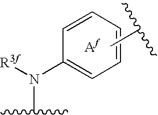

wherein each symbol is as defined above, is, for example,

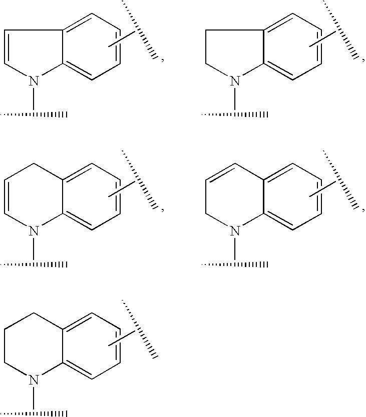

and the like.

The “ring structure” optionally has 1 to 5 (preferably 1 to 3, more preferably 1 or 2), the same or different substituents at any substitutable positions. As the substituents, substituents similar to the above-mentioned Substituent Group V can be mentioned.

R^{1f} and R^{2f} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{1f} and R^{2f} bonded to each other, for example,

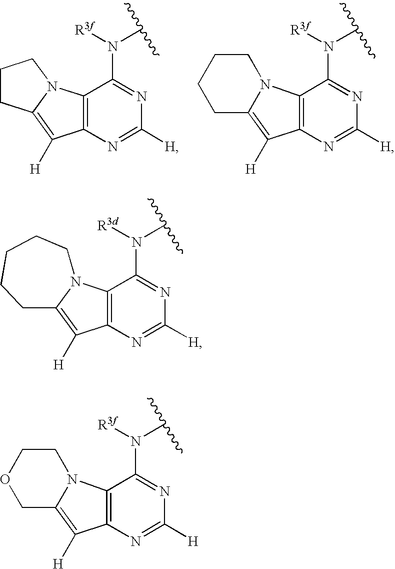

wherein each symbol is as defined above,
and the like can be mentioned.

R^{2f} and R^{3f} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{2f} and R^{3f} bonded to each other, for example,

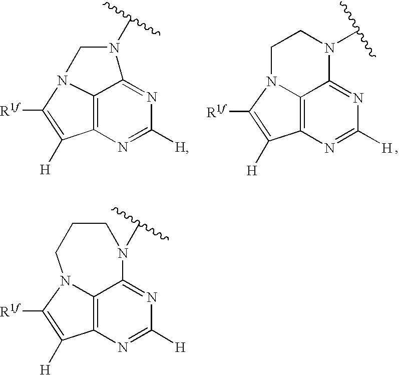

wherein each symbol is as defined above,
and the like can be mentioned.

The “ring structure” of the “optionally substituted ring structure” formed by R^{1f} and R^{2f}, or R^{2f} and R^{3f} optionally has 1 to 5 (preferably 1 to 3, more preferably 1 or 2), the same or different substituents at any substitutable positions. As the substituents, substituents similar to the above-mentioned Substituent Group V can be mentioned

The “benzene ring” of the “optionally substituted benzene ring” for Ring A^{f} optionally has 1 to 3, the same or different substituents at any substitutable positions. As the substituents, substituents similar to the above-mentioned Substituent Group V can be mentioned. Of these, halogen and methyl are preferable.

Ring A^{f} is preferably a benzene ring optionally substituted by substituent(s) selected from the group consisting of halogen and methyl.

The “piperidyl group” of the “optionally further substituted piperidyl group” for ring B^{f} optionally has, besides R^{4f}, 1 to 3, the same or different substituents at any substitutable positions. As the substituents, substituents similar to the above-mentioned Substituent Group V can be mentioned.

The “C₁₋₆ alkyl group” of the “optionally substituted C₁₋₆ alkyl group” for R^{4f} optionally has 1 to 5, the same or different substituents at any substitutable positions. As the substituents, substituents similar to the above-mentioned Substituent Group U can be mentioned.

The “C₅₋₈ cycloalkyl group” of the “optionally substituted C₅₋₈ cycloalkyl group” for R^{4f} optionally has 1 to 5, the same or different substituents at any substitutable positions. As the substituents, substituents similar to the above-mentioned Substituent Group V can be mentioned.

In the above-mentioned formula, the partial structural formula

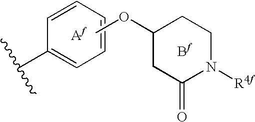

is preferably

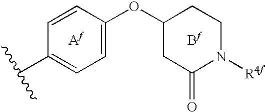

wherein each symbol is as defined above.

As specific examples, a compound represented by the following formula (If′) or a salt thereof (in the present specification, hereinafter sometimes to be abbreviated as “compound (If′)”) can be mentioned:

### [Compound (If′)]

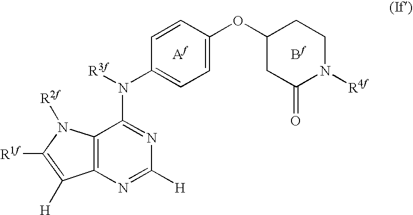 [1728] wherein each symbol is as defined above.

As preferable embodiment of compound (If), compound (If) wherein
R^{1f} is a hydrogen atom, a cyano group or an optionally halogenated C₁₋₆alkyl group,
R^{2f} is
(i) a C₁₋₆ alkyl group, or
(ii) a C₁₋₆ alkyl group substituted by substituent(s) selected from the group consisting of
(a) —NR^{6f}—CO—(CH₂)ₙ—SO₂-optionally halogenated C₁₋₄ alkyl,
(b) —NR^{6f}—CO—(CH₂)ₙ—OH,
(c) —O—(CH₂)ₙ—OH, and
(d) hydroxy
wherein n is an integer of 1 to 4, R^{6f} is a hydrogen atom or a C₁₋₄ alkyl group, and —(CH₂)ₙ— is optionally substituted by C₁₋₄ alkyl,
R^{3f} is a hydrogen atom or a C₁₋₆ alkyl group,
ring A^{f} is a benzene ring optionally substituted by substituent(s) selected from the group consisting of halogen and methyl,
ring B^{f} is a piperidyl group, and
R^{4f} is (i) an optionally substituted C₁₋₆ alkyl group, or (ii) an optionally substituted C₅₋₈ cycloalkyl group, can be mentioned.

As another preferable embodiment of compound (If), compound (If) wherein
R^{3f} is a hydrogen atom, and
ring A^{f} is a benzene ring optionally substituted by substituent(s) selected from the group consisting of halogen and methyl,
can be mentioned.

### [Compound (Ig)]

The present invention provides also a compound represented by the formula (Ig) or a salt thereof (in the present specification, hereinafter sometimes to be abbreviated as “compound (Ig)”).

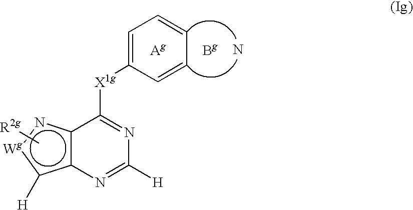

wherein each symbol is as defined above.

In the formula (Ig), the “benzene ring” of the “optionally substituted benzene ring” for ring A^{g} optionally has 1 to 3, the same or different substituents at any substitutable positions. As the substituents, substituents similar to the above-mentioned Substituent Group V can be mentioned.

As the “nitrogen-containing heterocycle” of the “optionally substituted nitrogen-containing heterocycle” for ring B^{g}, for example, a 3 to 8-membered (preferably 5 or 6-membered) aromatic heterocycle or a saturated or unsaturated (preferably saturated) aliphatic heterocycle and the like can be mentioned. Of these, 3 to 8-membered (preferably 5 or 6-membered) saturated or unsaturated (preferably saturated) aliphatic heterocyclic groups such as azetidine, pyrrolidine, piperidine, homopiperidine, heptamethylenimine, morpholine, thiomorpholine, piperazine, homopiperazine and the like, and the like can be preferably used.

The “nitrogen-containing heterocycle” optionally has 1 to 5, the same or different substituents at any substitutable positions. As the substituent, substituents similar to the above-mentioned Substituent Group V can be mentioned.

As the “aliphatic hydrocarbon group” of the “optionally substituted aliphatic hydrocarbon group” for R^{3g}, a C₁₋₆ alkyl group is preferable.

The “C₁₋₄ alkylene” and “—O—(C₁₋₄ alkylene)-” of the “C₁₋₄ alkylene or —O—(C₁₋₄ alkylene)-, each of which is optionally substituted,” for Y^{1g} are optionally substituted by 1 to 3 substituents selected form halogen, hydroxy, C₁₋₄ alkoxy, C₁₋₄ alkyl-carbonyl, carboxy, C₁₋₄ alkoxy-carbonyl, cyano, carbamoyl, sulfamoyl, nitro, amino, C₁₋₄ alkyl-carbonylamino, C₁₋₄ alkoxy-carbonylamino and C₁₋₄ alkylsulfonylamino.

X^{1g} is preferably —NR^{3g}—. In the formula, R^{3g} is preferably a hydrogen atom or a C₁₋₆ alkyl group, more preferably a hydrogen atom.

W^{g} is preferably C(R^{1g}).

As the “optionally substituted group bonded via a carbon atom, a nitrogen atom or an oxygen atom” for R^{1g}, a cyano group and an optionally substituted C₁₋₈ alkyl group are preferable. As the C₁₋₈ alkyl group, a C₁₋₆ alkyl group is preferable.

As the substituents for the alkyl group, substituents similar to the above-mentioned Substituent Group X can be mentioned. Of these, halogen is preferable.

R^{1g} is preferably a hydrogen atom, a halogen atom, a cyano group or an optionally halogenated C₁₋₆ alkyl group, more preferably a hydrogen atom.

As the “optionally substituted group bonded via a carbon atom or a sulfur atom” for R^{2g}, an optionally substituted C₁₋₈ alkyl group is preferable. As the C₁₋₈ alkyl group, a C₁₋₆ alkyl group is preferable.

As the substituents for the alkyl group, substituents similar to the above-mentioned Substituent Group X can be mentioned, preferably, substituent(s) selected from the group consisting of
(a) —O—(CH₂)ₙ—OH,
(b) —NR^{5g}—CO—(CH₂)ₙ—OH,
(c) —NR^{5g}—CO—(CH₂)ₙ—SO₂-optionally halogenated C₁₋₄ alkyl,
(d) hydroxy, and
(e) amino
wherein n is an integer of 1 to 4, R^{5g} is a hydrogen atom or a C₁₋₄ alkyl group, and —(CH₂)ₙ— is optionally substituted by C₁₋₄ alkyl,
can be used.

As the “ring structure” of the “optionally substituted ring structure” formed by R^{3g} bonded to the carbon atom on the benzene ring for ring A^{g}, a saturated or unsaturated (preferably saturated) 4 to 8-membered (preferably 5 or 6-membered) nitrogen-containing heterocycle can be mentioned.

Specifically,

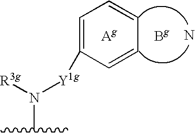

wherein each symbol is as defined above, is, for example,

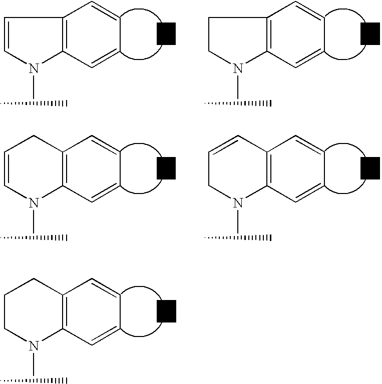

and the like.

The “ring structure” optionally has 1 to 5 (preferably 1 to 3, more preferably 1 or 2), the same or different substituents at any substitutable positions. As the substituents, substituents similar to the above-mentioned Substituent Group V can be mentioned.

R^{1g} and R^{2g} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{1g} and R^{2g} bonded to each other, for example,

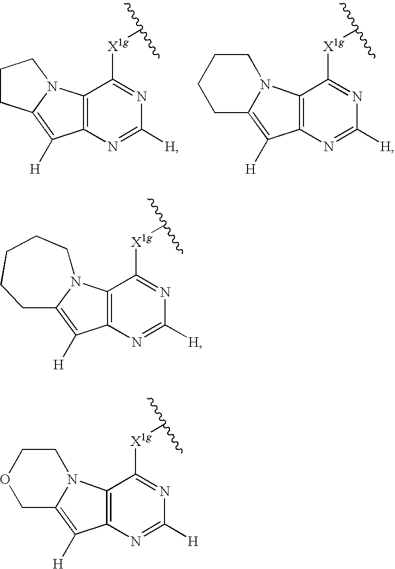

wherein each symbol is as defined above,
and the like can be mentioned.

R^{2g}and R^{3g} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{2g} and R^{3g} bonded to each other, for example,

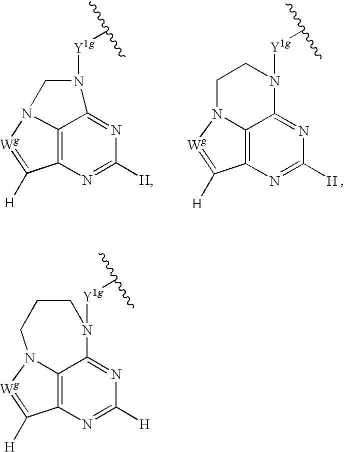

wherein each symbol is as defined above,
and the like can be mentioned.

The “ring structure” of the “optionally substituted ring structure” formed by R^{1g} and R^{2g}, or R^{2g} and R^{3g} optionally has 1 to 5 (preferably 1 to 3, more preferably 1 or 2), the same or different substituents at any substitutable positions. As the substituents, substituents similar to the above-mentioned Substituent Group V can be mentioned

When W^{g} is C(R^{1g}), compound (Ig) is represented by the following formula (IgA):

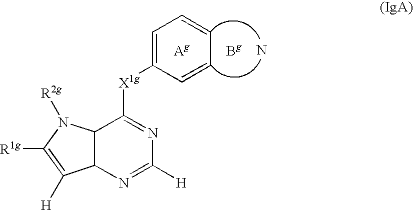

wherein each symbol is as defined above.

When W^{g} is N, compound (Ig) is represented by the following formula (IgB) or (IgC):

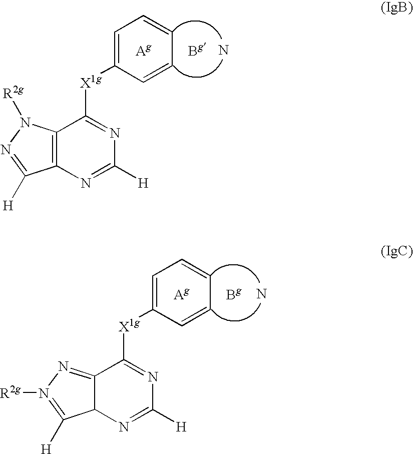

wherein each symbol is as defined above.

### [Compound (Iga)]

As preferable embodiment of compound (Ig), a compound represented by the following formula (Iga) or a salt thereof (in the present specification, hereinafter sometimes to be abbreviated as “compound (Iga)”) can be mentioned:

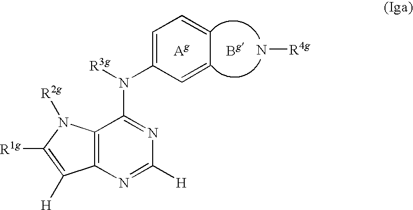

wherein R^{4g} is an optionally substituted hydrocarbon group, ring B^{g′} is a 5 or 6-membered nitrogen-containing heterocycle optionally further substituted besides R^{4g}, and the other symbols are as defined above.

In the above-mentioned formula (Iga), as the “5 or 6-membered nitrogen-containing heterocycle” of the “optionally further substituted 5 or 6-membered nitrogen-containing heterocycle” for ring B^{g′}, a 5 or 6-membered “nitrogen-containing heterocycle” from the “nitrogen-containing heterocycle” of the “optionally substituted nitrogen-containing heterocycle” for ring B^{g} can be mentioned.

R^{4g} is preferably (i) an optionally substituted C₆₋₁₄ aryl-C₁₋₈ alkyl group, (ii) an optionally substituted heterocyclyl-C₁₋₈ alkyl group, (iii) a C₁₋₈ alkyl group, or (iv) an optionally substituted C₆₋₁₄ aryl group, more preferably (i) a C₆₋₁₄ aryl-C₁₋₈ alkyl group optionally substituted by substituents) selected from the group consisting of halogen, C₁₋₆ alkyl-carbamoyl and halo C₁₋₆ alkoxy, (ii) an optionally substituted heterocyclyl-C₁₋₈ alkyl group, or (iii) an optionally substituted C₆₋₁₄ aryl group.

The “C₆₋₁₄ aryl-C₁₋₈ alkyl group” of the “optionally substituted C₆₋₁₄ aryl-C₁₋₈ alkyl group” for R^{4g}, “heterocyclyl-C₁₋₈ alkyl group” of the “optionally substituted heterocyclyl-C₁₋₈ alkyl group” for R^{4g} and “C₆₋₁₄ aryl group” of the “optionally substituted C₆₋₁₄ aryl group” for R^{4g} optionally have 1 to 5, the same or different substituents at any substitutable positions. As the substituents, substituents similar to the above-mentioned Substituent Group V can be mentioned.

In the above-mentioned formula, the partial structural formula

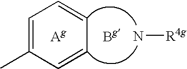

is preferably

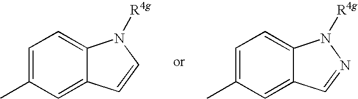

wherein each symbol is as defined above.

As preferable embodiment of compound (Ig), compound (Iga) wherein, in the above-mentioned formula (Iga),
R^{1g} is a hydrogen atom, a halogen atom, a cyano group or an optionally halogenated C₁₋₆ alkyl group,
R^{2g} is a hydrogen atom or an optionally substituted C₁₋₆ alkyl group,
R^{3g} is a hydrogen atom or a C₁₋₆ alkyl group,
R^{4g} is (i) an optionally substituted C₆₋₁₄ aryl-C₁₋₈ alkyl group, (ii) an optionally substituted heterocyclyl-C₁₋₈ alkyl group, (iii) a C₁₋₈ alkyl group, or (iv) an optionally substituted C₆₋₁₄ aryl group,
can be mentioned.

As another more preferable embodiment of compound (Ig), compound (Iga) wherein, in the above-mentioned formula (Iga),
R^{1g} is a hydrogen atom, a halogen atom, a cyano group or an optionally halogenated C₁₋₆ alkyl group,
R^{2g} is
(i) a hydrogen atom,
(ii) a C₁₋₆ alkyl group, or
(iii) a C₁₋₆ alkyl group substituted by substituent(s) selected from the group consisting of
(a) —O—(CH₂)ₙ—OH,
(b) —NR^{5g}—CO—(CH₂)ₙ—OH,
(c) —NR^{5g}—CO—(CH₂)ₙ—SO₂-optionally halogenated C₁₋₄ alkyl,
(d) hydroxy, and
(e) amino
wherein n is an integer of 1 to 4, R^{5g} is a hydrogen atom or a C₁₋₄ alkyl group, and —(CH₂)ₙ— is optionally substituted by C₁₋₄ alkyl,
R^{3g} is a hydrogen atom or a C₁₋₆ alkyl group,

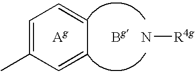

is the formula

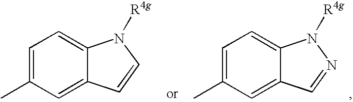

and
R^{4g} is (i) a C₆₋₁₄ aryl-C₁₋₈ alkyl group optionally substituted by substituent(s) selected from the group consisting of halogen, C₁₋₆ alkyl-carbamoyl and halo C₁₋₆ alkoxy, (ii) an optionally substituted heterocyclyl-C₁₋₈ alkyl group, or (iii) an optionally substituted C₆₋₁₄ aryl group,
can be mentioned.

As compound (Ig), particularly preferably,
N-[2-(4-{[1-(3-fluorobenzyl)-1H-indazol-5-yl]amino}-5H-pyrrolo[3,2-d]pyrimidin-5-yl)ethyl]-2-(methylsulfonyl)acetamide,
N-[2-(4-{[1-(3-fluorobenzyl)-1H-indol-5-yl]amino}-5H-pyrrolo[3,2-d]pyrimidin-5-yl)ethyl]-3-hydroxy-3-methylbutanamide,
N-(tert-butyl)-3-[(5-{[5-(2-hydroxyethyl)-5H-pyrrolo[3,2-d]pyrimidin-4-yl]amino}-1H-indol-1-yl)methyl]benzamide,
N-(tert-butyl)-3-[(5-{[5-(2-hydroxyethyl)-5H-pyrrolo[3,2-d]pyrimidin-4-yl]amino}-1H-indazol-1-yl)methyl]benzamide, and
N-(tert-butyl)-6-[(5-{[5-(2-hydroxyethyl)-5H-pyrrolo[3,2-d]pyrimidin-4-yl]amino}-1H-indol-1-yl)methyl]pyridine-2-carboxamide,
and salts thereof can be mentioned.

### [Compound (Ih)]

The present invention provides also a compound represented by the formula (Ih) or a salt thereof (in the present specification, hereinafter sometimes to be abbreviated as “compound (Ih)”).

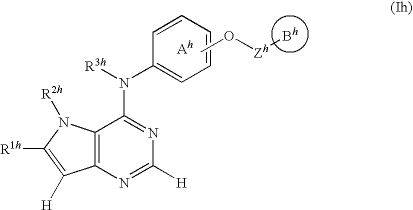

wherein each symbol is as defined above.

In the above-mentioned formula (Ih), as the “optionally substituted group bonded via a carbon atom or a sulfur atom” for R^{2h}, an optionally substituted C₁₋₈ alkyl group is preferable. As the C₁₋₈ alkyl group, a C₁₋₆ alkyl group is preferable.

As the substituents for the alkyl group, substituents similar to the above-mentioned Substituent Group X can be mentioned, preferably, substituent(s) selected from the group consisting of
(a) —O—(CH₂)ₙ—OH,
(b) —NR^{5g}—CO—(CH₂)ₙ—OH,
(c) —NR^{5g}—CO—(CH₂)ₙ—SO₂-optionally halogenated C₁₋₄ alkyl, and
(d) hydroxy,
wherein n is an integer of 1 to 4, R^{6h} is a hydrogen atom or a C₁₋₄ alkyl group, and —(CH₂)ₙ— is optionally substituted by C₁₋₄ alkyl,
can be used.

As the “aliphatic hydrocarbon group” of the “optionally substituted aliphatic hydrocarbon group” for R^{3h}, a C₁₋₆ alkyl group is preferable.

R^{3h} is preferably a hydrogen atom or a C₁₋₆ alkyl group, more preferably a hydrogen atom.

As the “ring structure” of the “optionally substituted ring structure” formed by R^{3h} bonded to the carbon atom on the adjacent benzene ring, a saturated or unsaturated (preferably saturated) 4 to 8-membered (preferably 5 or 6-membered) nitrogen-containing heterocycle can be mentioned.

Specifically,

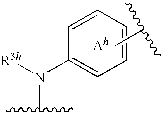

wherein each symbol is as defined above, is, for example,

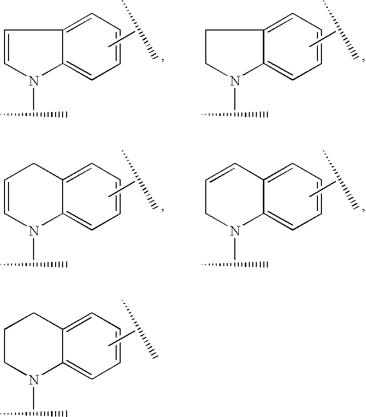

and the like.

The “ring structure” optionally has 1 to 5 (preferably 1 to 3, more preferably 1 or 2), the same or different substituents at any substitutable positions. As the substituents, substituents similar to the above-mentioned Substituent Group V can be mentioned.

R^{1h} and R^{2h} are optionally bonded to each other to form an optionally substituted ring structure. As the “ring structure”, a saturated or unsaturated (preferably saturated) 4 to 8-membered (preferably 5- to 7-membered) heterocycle can be mentioned.

As the “ring structure” of the “optionally substituted ring structure” formed by R^{1h} and R^{2h} bonded to each other, for example,

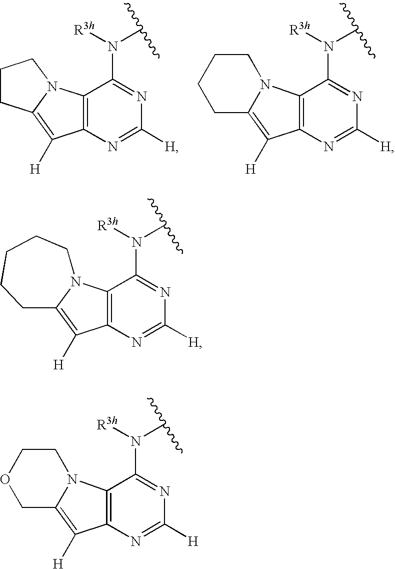

wherein each symbol is as defined above,
and the like can be mentioned.

R^{2h} and R^{3h} are optionally bonded to each other to form an optionally substituted ring structure. As the “ring structure”, a saturated or unsaturated (preferably saturated). 4 to 8-membered (preferably 5- to 7-membered) heterocycle can be mentioned.

As the “ring structure” of the “optionally substituted ring structure” formed by R^{2h} and R^{3h} bonded to each other, for example,

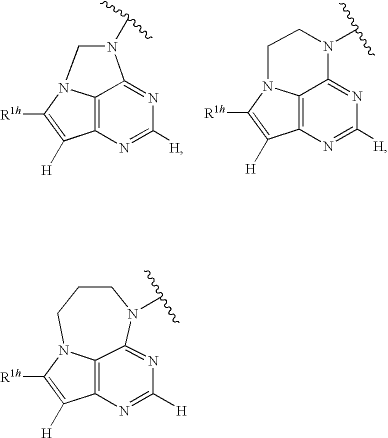

wherein each symbol is as defined above,
and the like can be mentioned.

The “ring structure” of the “optionally substituted ring structure” formed by R^{1h} and R^{2h}, or R^{2h} and R^{3h} optionally has 1 to 5 (preferably 1 to 3, more preferably 1 or 2), the same or different substituents at any substitutable positions. As the substituents, substituents similar to the above-mentioned Substituent Group V can be mentioned

The “benzene ring” of the “optionally substituted benzene ring” for ring A^{h} optionally has 1 to 3, the same or different substituents at any substitutable positions. As the substituents, substituents similar to the above-mentioned Substituent Group V can be mentioned. Of these, halogen and methyl are preferable.

Ring A^{h} is preferably a benzene ring optionally substituted by substituent(s) selected from the group consisting of halogen and methyl.

As the “C₁₋₃ alkylene” of the “optionally substituted C₁₋₃ alkylene” for Z^{h}, methylene is preferable.

The “C₁₋₃ alkylene” of the “optionally substituted C₁₋₃ alkylene” for Z^{h} is optionally substituted by 1 to 3 substituents selected from the group consisting of halogen, hydroxy, C₁₋₄ alkoxy, C₁₋₄ alkyl-carbonyl, carboxy, C₁₋₄ alkoxy-carbonyl, cyano, carbamoyl, sulfamoyl, nitro, amino, C₁₋₄ alkyl-carbonylamino, C₁₋₄ alkoxy-carbonylamino and C₁₋₄ alkylsulfonylamino.

As the “C₆₋₁₄ aryl group” of the “optionally substituted C₆₋₁₄ aryl group” for ring B^{h}, a phenyl group is preferable.

As the “heterocyclic group” of the “optionally substituted heterocyclic group” for ring B^{h}, a pyridyl group and a piperidyl group are preferable.

As the “C₅₋₈ cycloalkyl group” of the “optionally substituted C₅₋₈ cycloalkyl group” for ring B^{h}, a cyclohexyl group is preferable.

The “C₆₋₁₄ aryl group” of the “optionally substituted C₆₋₁₄ aryl group” for ring B^{h}, the “heterocyclic group” of the “optionally substituted heterocyclic group” for ring B^{h} and the “C₅₋₈ cycloalkyl group” of the “optionally substituted C₅₋₈ cycloalkyl group” for ring B^{h} optionally have 1 to 5, the same or different substituents at any substitutable positions. As the substituents, substituents similar to the above-mentioned Substituent Group V can be mentioned.

In the above-mentioned formula, the partial structural formula

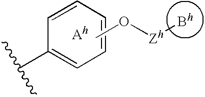

is preferably

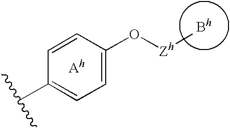

wherein each symbol is as defined above.

As specific examples, a compound represented by the following formula (Ih′) or a salt thereof (in the present specification, hereinafter sometimes to be abbreviated as “compound (Ih′)”) can be mentioned:

### [Compound (Ih′)]

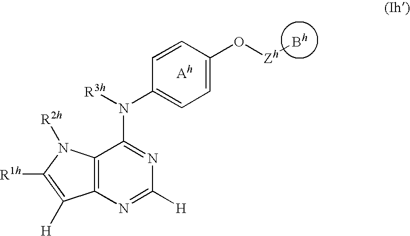 [1863] wherein each symbol is as defined above.

As preferable embodiment of compound (Ih), a compound represented by the following formula (Iha) or a salt thereof (in the present specification, hereinafter sometimes to be abbreviated as “compound (Iha)”) can be mentioned:

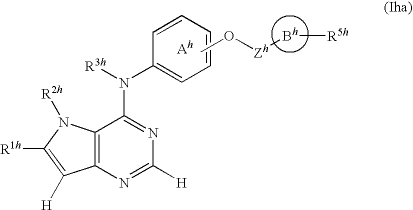

wherein
R^{5h} is
(i) an optionally substituted amino group,
(ii) an optionally substituted carbamoyl group,
(iii) an optionally substituted ureido group,
(iv) an optionally substituted sulfamoyl group,
(v) an optionally substituted heterocyclic group,
(vi) an optionally substituted hydrocarbon group,
(vii) a halogen atom, or
(viii) an optionally substituted carboxyl group, and ring B^{h′} is (i) a C₆₋₁₄ aryl group, (ii) a heterocyclic group, or (iii) a C₅₋₈ cycloalkyl group, each of which is optionally further substituted besides R^{5h}, and the other symbols are as defined above.

In the above-mentioned formula (Iha), as the “optionally substituted amino group” for R^{5h}, an amino group, a C₁₋₆ alkyl-amino group, an optionally halogenated C₁₋₆ alkanoyl-amino group, a hydroxy-C₁₋₆ alkanoyl-amino group, a C₁₋₆ alkanoyl-amino group having hydroxy and halogen, a C₃₋₇ cycloalkyl-C₁₋₆ alkanoyl-amino group, a C₁₋₆ alkanoyl-amino group having C₃₋₇ cycloalkyl and halogen, a C₁₋₆ alkylsulfonyl-C₁₋₆ alkanoyl-amino group, a C₃₋₇ cycloalkyl-carbonyl-amino group and a C₁₋₆ alkoxy-carbonyl-amino group are preferable.

As the “optionally substituted carbamoyl group” for R^{5h}, a carbamoyl group, an optionally halogenated C₁₋₆ alkyl-carbamoyl group, a hydroxy-C₁₋₆ alkyl-carbamoyl group, a C₁₋₆ alkoxy-C₁₋₆ alkyl-carbamoyl group, a C₃₋₇ cycloalkyl-carbamoyl group, and a 5 or 6-membered cyclic amino-carbonyl group optionally containing an oxygen atom are preferable.

As the “optionally substituted ureido group” for R^{5h}, a ureido group, a C₁₋₆ alkyl-ureido group, a C₃₋₇ cycloalkyl-ureido group, and a 5- to 8-membered heterocyclyl-ureido group containing, besides carbon atoms, 1 to 3 hetero atoms selected from the group consisting of a nitrogen atom, an oxygen atom and a sulfur atom are preferable.

As the “optionally substituted sulfamoyl group” for R^{5h}, a sulfamoyl group optionally substituted by C₁₋₆ alkyl is preferable.

As the “optionally substituted heterocyclic group” for R^{5h}, a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl is preferable.

As the “optionally substituted hydrocarbon group” for R^{5h}, an optionally halogenated C₁₋₆ alkyl group and a C₁₋₆ alkoxy-carbonyl group are preferable.

As the “optionally substituted carboxyl group” for R^{5h}, a carboxyl group is preferable.

In the above-mentioned formula (Iha), as the “C₆₋₁₄ aryl group” of the “optionally substituted C₆₋₁₄ aryl group” for ring B^{h′}, a phenyl group is preferable.

As the “heterocyclic group” of the “optionally substituted heterocyclic group” for ring B^{h′}, a pyridyl group and a piperidyl group are preferable.

As the “C₅₋₈ cycloalkyl group” of the “optionally substituted C₅₋₈ cycloalkyl group” for ring B^{h′}, a cyclohexyl group is preferable.

The “C₆₋₁₄ aryl group” of the “optionally substituted C₆₋₁₄ aryl group” for ring B^{h′}, the “heterocyclic group” of the “optionally substituted, heterocyclic group” for ring B^{h′} and the “C₅₋₈ cycloalkyl group” of the “optionally substituted C₅₋₈ cycloalkyl group” for ring B^{h′} optionally have besides R^{5h}, 1 to 5, the same or different substituents, at any substitutable positions. As the substituents, substituents similar to the above-mentioned Substituent Group V can be mentioned.

Ring B^{h′} is preferably a phenyl group, a pyridyl group or a piperidyl group, each of which is optionally further substituted besides R^{5h}.

As more preferable embodiment of compound (Ih), compound (Iha) wherein, in the above-mentioned formula (Iha),
R^{1h} is a halogen atom or an optionally halogenated C₁₋₆ alkyl group,
R^{2h} is
(i) a hydrogen atom,
(ii) a C₁₋₆ alkyl group, or
(iii) a C₁₋₆ alkyl group substituted by substituent(s) selected from the group consisting of
(a) —O—(CH₂)ₙ—OH,
(b) —NR^{6h}—CO—(CH₂)ₐ—OH,
(c) —NR^{6h}—CO—(CH₂)ₙ—SO₂-optionally halogenated C₁₋₄ alkyl, and
(d) hydroxy
wherein n is an integer of 1 to 4, R^{6h} is a hydrogen atom or a C₁₋₄ alkyl group, and —(CH₂)ₙ— is optionally
substituted by C₁₋₄ alkyl,
R^{3h} is a hydrogen atom or a C₁₋₆ alkyl group,
Z^{h} is a bond or methylene,
ring A^{h} is a benzene ring optionally substituted by substituent(s) selected from the group consisting of halogen and methyl,
R^{5h} is
(i) an amino group,
(ii) a C₁₋₆ alkyl-amino group,
(iii) an optionally halogenated C₁₋₆ alkanoyl-amino group,
(iv) a hydroxy-C₁₋₆ alkanoyl-amino group,
(v) a C₁₋₆ alkanoyl-amino group having hydroxy and halogen,
(vi) a C₃₋₇ cycloalkyl-C₁₋₆ alkanoyl-amino group,
(vii) a C₁₋₆ alkanoyl-amino group having C₃₋₇ cycloalkyl and halogen,
(viii) a C₁₋₆ alkylsulfonyl-C₁₋₆ alkanoyl-amino group,
(ix) a C₃₋₇ cycloalkyl-carbonyl-amino group,
(x) a C₁₋₆ alkoxy-carbonyl-amino group,
(xi) a carbamoyl group,
(xii) an optionally halogenated C₁₋₆ alkyl-carbamoyl group,
(xiii) a hydroxy-C₁₋₆ alkyl-carbamoyl group,
(xiv) a C₁₋₆ alkoxy-C₁₋₆ alkyl-carbamoyl group,
(xv) a C₃₋₇ cycloalkyl-carbamoyl group,
(xvi) a 5 or 6-membered cyclic amino-carbonyl group optionally containing an oxygen atom,
(xvii) a ureido group,
(xviii) a C₁₋₆ alkyl-ureido group,
(xix) a C₃₋₇ cycloalkyl-ureido group,
(xx) a 5- to 8-membered heterocyclyl-ureido group containing, besides carbon atoms, 1 to 3 hetero atoms selected from the group consisting of a nitrogen atom, an oxygen atom and a sulfur atom,
(xxi) a sulfamoyl group optionally substituted by C₁₋₆ alkyl,
(xxii) a 5- to 8-membered heterocyclic group containing, besides carbon atoms, 1 to 3 hetero atoms selected from the group consisting of a nitrogen atom, an oxygen atom and a sulfur atom, which is optionally substituted by substituent(s) selected from the group consisting of optionally halogenated C₁₋₆ alkyl and C₁₋₆ alkoxy-carbonyl,
(xxiii) an optionally halogenated C₁₋₆ alkyl group,
(xxiv) a C₁₋₆ alkoxy-carbonyl group,
(xxv) a halogen atom, or
(xxvi) a carboxyl group, and
ring B^{h′} is a phenyl group, a pyridyl group or a piperidyl group, each of which is optionally further substituted besides R^{5h},
can be mentioned.

As compound (Ih), particularly preferably,
N-(3-{2-chloro-4-[(6-chloro-5-methyl-5H-pyrrolo[3,2-d]pyrimidin-4-yl)amino]phenoxy}phenyl)cyclopropanecarboxamide,
6-chloro-N-{3-chloro-4-[3-(trifluoromethyl)phenoxy]phenyl}-5-methyl-5H-pyrrolo[3,2-d]pyrimidine-4-amine,
N-[3-(2-chloro-4-{[6-chloro-5-(2-hydroxyethyl)-5H-pyrrolo[3,2-d]pyrimidin-4-yl]amino}phenoxy)phenyl]cyclopropanecarboxamide, and
N-(tert-butyl)-3-(2-chloro-4-{[6-chloro-5-(2-hydroxyethyl)-5H-pyrrolo[3,2-d]pyrimidin-4-yl]amino}phenoxy)benzamide,
and salts thereof can be mentioned.

As the salts of the compounds represented by the formulas, for example, metal salts, ammonium salts, salts with organic base, salts with inorganic acid, salts with organic acid, salts with basic or acidic amino acid and the like can be mentioned.

As preferable examples of the metal salt, for example, alkali metal salts such as sodium salt, potassium salt and the like; alkaline earth metal salts such as calcium salt, magnesium salt, barium salt and the like; aluminum salt and the like can be mentioned.

As preferable examples of the salts with organic base, for example, salts with trimethylamine, triethylamine, pyridine, picoline, 2,6-lutidine, ethanolamine, diethanolamine, triethanolamine, tromethamine[tris(hydroxymethyl)methylamine], t-butylamine, cyclohexylamine, dicyclohexylamine, N,N′-dibenzylethylenediamine and the like can be mentioned.

As preferable examples of salts with inorganic acid, for example, salts with hydrochloric acid, hydrobromic acid, nitric acid, sulfuric acid, phosphoric acid and the like can be mentioned.

As preferable examples of the salts with organic acid, for example, salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like can be mentioned.

As preferable examples of the salts with basic amino acid, for example, salts with arginine, lysine, ornithine and the like can be mentioned.

As preferable examples of the salts with acidic amino acid, for example, salts with aspartic acid, glutamic acid and the like can be mentioned.

Of these, pharmaceutically acceptable salts are preferable. When a compound contains an acidic functional group, for example, inorganic salts such as alkali metal salts (e.g., sodium salt, potassium salt etc.), alkaline earth metal salts (e.g., calcium salt, magnesium salt, barium salt etc.) and the like, ammonium salt and the like can be mentioned. And when a compound contains a basic functional group, for example, salts with inorganic acid such as hydrochloric acid, hydrobromic acid, nitric acid, sulfuric acid, phosphoric acid and the like, and salts with organic acid such as acetic acid, phthalic acid, fumaric acid, oxalic acid, tartaric acid, maleic acid, citric acid, succinic acid, methanesulfonic acid, benzenesulfonic acid, p-toluenesulfonic acid and the like can be mentioned.

### [Production Methods]

Hereinafter the production methods of the compounds (Ia) to (Ih) of the present invention are explained.

### [Production Method A]

Compound (Ia) of the present invention can be obtained by, for example, the method shown by the following scheme or a method analogous thereto and the like.

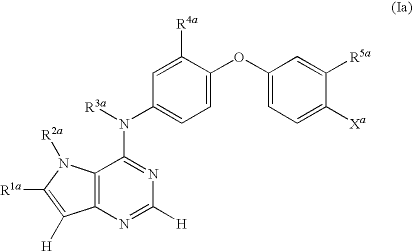

wherein each symbol is as defined above.

Each compound in the following schemes includes salts, and as such salts, for example, those similar to the salts of compound (Ia) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (Ia) of the present invention can be produced, for example, by reacting a compound represented by the formula:

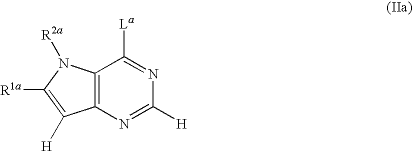

wherein L^{a} is a leaving group, and the other symbols are as defined above,
or a salt thereof and a compound represented by the formula:

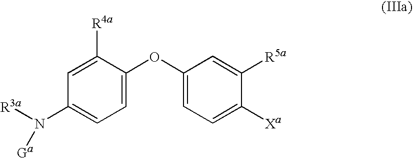

wherein G^{a} is a hydrogen atom or a metal atom, and the other symbols are as defined above,
or a salt thereof.

G^{a} is mainly a hydrogen atom, but may be an alkali metal such as lithium, sodium, potassium, cesium and the like, or an alkaline earth metal such as magnesium, calcium and the like.

Compound (IIIa) or a salt thereof is preferably used in an amount of 1-5 equivalents, preferably 1-2 equivalents, relative to compound (IIa) and the reaction is preferably carried out in a solvent. In addition, a base or an ammonium salt may be used in an amount of about 1-10 equivalents, preferably 1-2 equivalents.

In the aforementioned formula, as the leaving group for L^{a}, a halogen atom such as chlorine, bromine, iodine and the like, a group represented by the formula: —S(O)ₖR^{z} wherein k is an integer of 0, 1 or 2, and R^{z} is a lower (C₁₋₄)alkyl group such as methyl, ethyl, propyl and the like, a C₆₋₁₀ aryl group such as phenyl, tolyl and the like, or a group represented by the formula: —OR^{z} wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

A compound within the scope of the present invention can be also produced by applying means known per se to the obtained compound (Ia) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (Ia), which is a product of the reaction, may be produced as a single compound or as a mixture.

The compound (Ia) of the present invention thus obtained can be subjected to a means known per se, such as solvent extraction, concentration, neutralization, filtration, crystallization, recrystallization, column chromatography, high performance liquid chromatography and the like, whereby the objective compound can be isolated and purified at high purity from a reaction mixture.

As the starting compound (IIIa) of this production method, a commercially available one is used or can be produced by a means known per se.

The starting compound (IIa) of this production method can be produced by, for example, a method shown by the following scheme. Here, compounds (IIaa), (IIab), (IIac) and (IIad) are encompassed in compound (IIa).

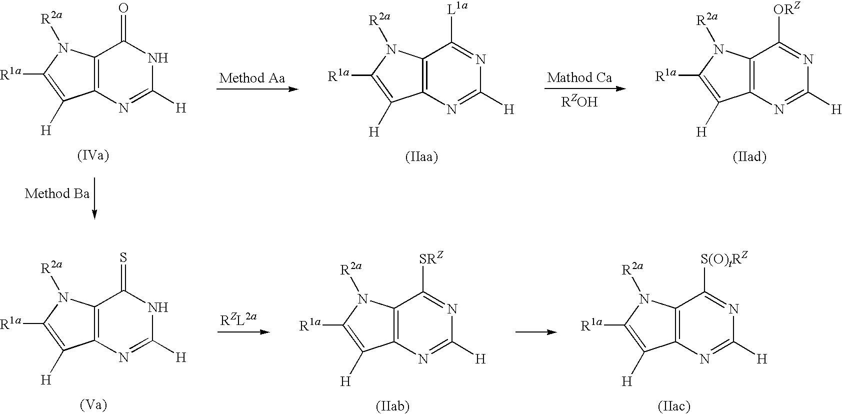

wherein L^{1a} and L^{2a} are halogen atoms, R^{z} is as defined above, and t is an integer of 1 or 2.

As Method Aa, compound (IIaa) can be produced by reacting compound (IVa) with a halogenating agent. As Method Ba, compound (IVa) is reacted with a thionating agent to give compound (Va), which is then reacted with a compound represented by R^{z}L^{2a} in the presence of a base to give compound (IIab), which is further subjected to an oxidation reaction to give compound (IIac). As Method Ca, compound (IIaa) is reacted with a compound represented by R^{z}OH in the presence of a base to give compound (IIad).

As the halogenating agent in Method Aa, for example, about 1-100 equivalents of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons, such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1-20 hr, preferably about 1-10 hr.

As the thionating agent used in the production step from compound (IVa) to compound (Va) in Method Ba, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and the reaction time is generally about 1-20 hr, preferably about 1-10 hr.

As R^{z}L^{2a} in the production step from compound (Va) to compound (IIab) in Method Ba, for example, about 1-5 equivalents of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

As the oxidizing agent in the production step from compound (IIab) to compound (IIac) in Method Ba, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIac) wherein t=1 is produced, the oxidizing agent is used in about 1-1.5 equivalents relative to compound (IIab), and when compound (IIac) wherein t=2 is produced, it is used in about 2-3 equivalents relative to compound (IIab). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and, the reaction time is generally about 1-20 hr, preferably about 1-10 hr.

As R^{z}OH in the production step from compound (IIaa) to compound (IIad) in Method Ca, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Furthermore, compound (IVa) can be produced by, for example, a method shown by the following formula:

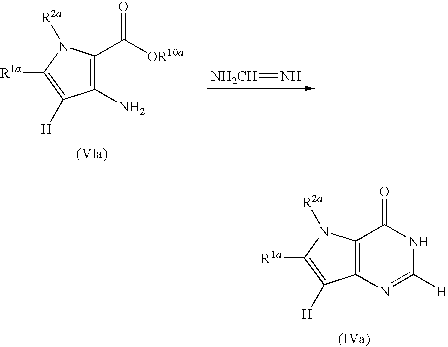

wherein R^{10a} is a C₁₋₄ alkyl group, and other symbols are as defined above.

That is, compound (VIa) is reacted with about 1-4 equivalents of formamidine or a salt thereof to give compound (IVa). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Compound (IIa) can be also produced by, for example, a method shown by the following formula:

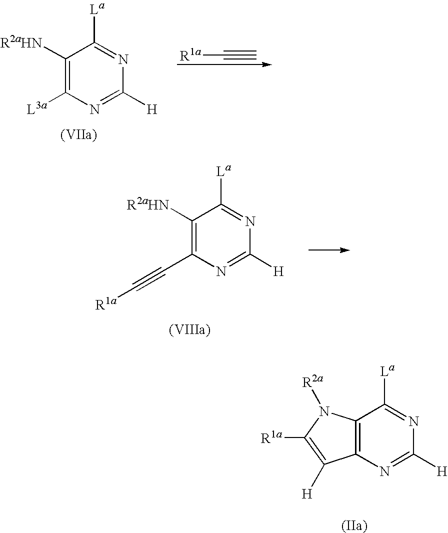

wherein L^{3a} is a halogen atom, and other symbols are as defined above.

For the production step from compound (VIIa) to compound (VIIIa) in this method, a reaction generally known as a Sonogashira reaction or a reaction analogous thereto can be carried out, and generally, compound (VIIIa) can be produced by reacting compound (VIIa) with about 1-3 equivalents of a compound represented by the formula:

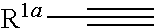

in the presence of a base, about 0.01-1 equivalent of a palladium catalyst and copper iodide. As the base, for example, triethylamine, N-ethyldiisopropylamine, diisopropylamine, pyridine, N,N-dimethylaminopyridine, diazabicycloundecene (DBU), sodium carbonate, potassium carbonate, sodium hydrogencarbonate, potassium hydrogencarbonate and the like can be used. As the palladium catalyst, for example, dichlorobis(triphenylphosphine)palladium(II), palladium on carbon, palladium(II) diacetate, bis(benzonitrile)dichloropalladium(II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried out at room temperature or under heating, and the reaction time is generally about 1-50 hr, preferably about 1-20 hr.

For the production step from compound (VIIIa) to Compound (IIa) in this method, a cyclization reaction is generally carried out in the presence of about 1-3 equivalents of base or about 0.01-1 equivalent of copper iodide to give compound (IIa). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine, pyridine, N,N-dimethylaminopyridine, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IIa), a starting compound (IIa) having a different substituent can be produced by substituent conversion from, as a starting material, compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IIa) can be also produced.

### [Production Method B]

Compound (Ib) of the present invention can be obtained by, for example, the method shown by the following schemes or a method analogous thereto and the like.

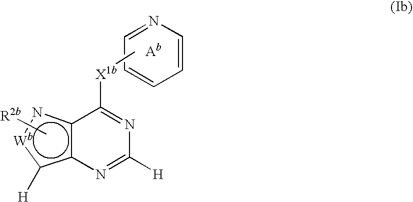

wherein each symbol is as defined above.

Each compound in the following schemes includes salts, and as such salts, for example, those similar to the salts of compound (Ib) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (Ib) of the present invention can be produced, for example, by reacting a compound represented by the formula:

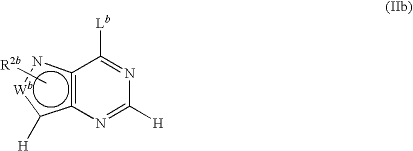

wherein L^{b} is a leaving group, and the other symbols, are as defined above;
or a salt thereof and a compound represented by the formula:

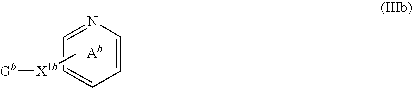

wherein G^{b} is a hydrogen atom or a metal atom, and the other symbols are as defined above,
or a salt thereof.

When X^{1b} is —NR^{3b}—Y^{1b}—, —O— or —S—, G^{b} is mainly a hydrogen atom, but it may be an alkali metal such as lithium, sodium, potassium, cesium and the like, or an alkaline earth metal such as magnesium, calcium and the like.

When X^{1b} is —CHR^{3b}—, G^{b} may be a metal such as lithium, halogenated magnesium, copper, zinc and the like.

Compound (IIIb) or a salt thereof is preferably used in an amount of 1-5 equivalents, preferably 1-2 equivalents, relative to compound (IIb) and the reaction is preferably carried out in a solvent. In addition, a base or an ammonium salt may be used in an amount of about 1-10 equivalents, preferably 1-2 equivalents.

In the aforementioned formula, as the leaving group for L^{b}, a halogen atom such as chlorine, bromine, iodine and the like, a group represented by the formula: —S(O)ₖR^{z} wherein k is an integer of 0, 1 or 2, and R^{z} is a lower (C₁₋₄)alkyl group such as methyl, ethyl, propyl and the like, a C₆₋₁₀ aryl group such as phenyl, tolyl and the like, or a group represented by the formula: —OR^{z} wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

Compound (Ib) wherein X^{1b} is —SO— or —SO₂— can be produced by subjecting compound (Ib) wherein X^{1b} is —S— to an oxidization reaction. As the oxidizing agent in the production step, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (Ib) wherein X^{1b} is —SO— is produced, the oxidizing agent is used in about 1-1.5 equivalents relative to the starting compound, and when compound (Ib) wherein X^{1b} is —SO₂— is produced, it is used in about 2-3 equivalents relative to the starting compound. The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

A compound within the scope of the present invention can be also produced by applying means known per se to the obtained compound (Ib) of the present invention for introduction of substituents and conversion of functional groups. For conversion of substituents, a known conventional method can be used. For example, conversion to carboxy group by hydrolysis of ester, conversion to carbamoyl group by amidation'of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (Ib), which is a product of the reaction, may be produced as a single compound or as a mixture.

The compound (Ib) of the present invention thus obtained can be subjected to a means known per se, such as solvent extraction, concentration, neutralization, filtration, crystallization, recrystallization, column chromatography, high performance liquid chromatography and the like, whereby the objective compound, can be isolated and purified at high purity from a reaction mixture.

As the starting compound (IIIb) of this production method, a commercially available one is used or can be produced by a means known per se.

The starting compound (IIb) of this production method can be produced by, for example, a method shown by the following scheme. Here, compounds (IIba), (IIbb), (IIbc), (IIbd) and (IIbe) are encompassed in compound

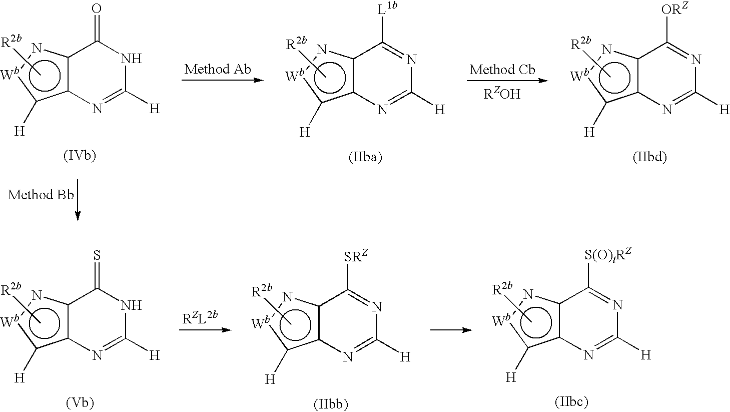

wherein L^{1b} and L^{2b} are halogen atoms, R^{z} is as defined above, and t is an integer of 1 or 2.

As Method Ab, compound (IIba) can be produced by reacting compound (IVb) with a halogenating agent. As Method Bb, compound (IVb) is reacted with a thionating agent to give compound (Vb), which is then reacted with a compound represented by R^{z}L^{2b}) in the presence of a base to give compound (IIbb), which is further subjected to an oxidation reaction to give compound (IIbc). As Method Cb, compound (IIba) is reacted with a compound represented by R^{z}OH in the presence of a base to give compound (IIbd).

As the halogenating agent in Method Ab, for example, about 1-100 equivalents of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1-20 hr, preferably about 1-10 hr.

As the thionating agent used in the production step from compound (IVb) to compound (Vb) in Method Bb, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and the reaction time is generally about 1-20 hr, preferably about 1-10 hr.

As R^{z}L^{2b} in the production step from compound (Vb) to compound (IIbb) in Method Bb, for example, about 1-5 equivalents of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

As the oxidizing agent in the production step from compound (IIbb) to compound (IIbc) in Method Bb, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIbc) wherein t=1 is produced, the oxidizing agent is used in about 1-1.5 equivalents relative to compound (IIbb), and when compound (IIbc) wherein t=2 is produced, it is used in about 2-3 equivalents relative to compound (IIbb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

As R^{z}OH in the production step from compound (IIba) to compound (IIbd) in Method Cb, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Furthermore, compound (IVb) can be produced by, for example, a method shown by the following formula:

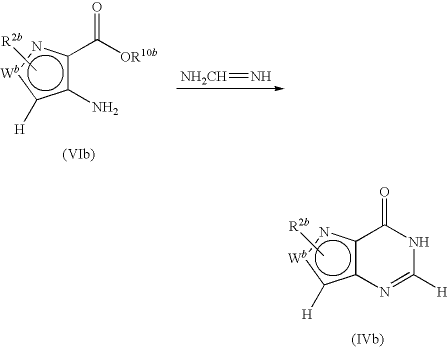

wherein R^{10b} is a C₁₋₄ alkyl group, and other symbols are as defined above.

That is, compound (VIb) is reacted with about 1-4 equivalents of formamidine or a salt thereof to give compound (IVb). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

When W^{b} is C(R^{1b}), compound (IIbe) can be also produced by, for example, a method shown by the following formula:

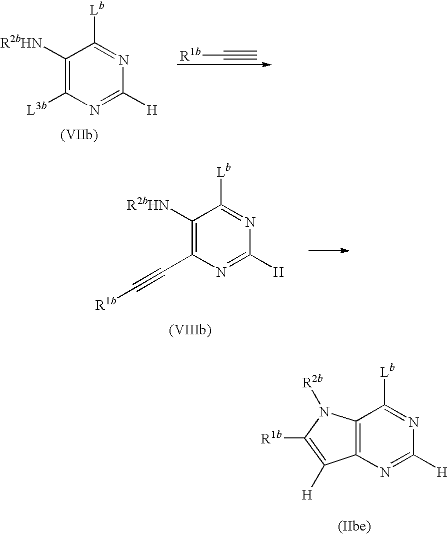

wherein L^{3b} is a halogen atom, and other symbols are as defined above.

For the production step from compound (VIIb) to compound (VIIIb) in this method, a reaction generally known as a Sonogashira reaction or a reaction analogous thereto can be carried out, and generally, compound (VIIIb) can be produced by reacting compound (VIIb) with about 1-3 equivalents of a compound represented by the formula:

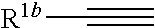

in the presence of a base, about 0.01-1 equivalent of a palladium catalyst and copper iodide. As the base, for example, triethylamine, N-ethyldiisopropylamine, diisopropylamine, pyridine, N,N-dimethylaminopyridine, diazabicycloundecene (DBU), sodium carbonate, potassium carbonate, sodium hydrogencarbonate, potassium hydrogencarbonate and the like can be used. As the palladium catalyst, for example, dichlorobis(triphenylphosphine)palladium(II), palladium on carbon, palladium(II) diacetate, bis(benzonitrile)dichloropalladium(II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried out at room temperature or under heating, and the reaction time is generally about 1-50 hr, preferably about 1-20 hr.

For the production step from compound (VIIIb) to compound (IIbe) in this method, a cyclization reaction is generally carried out in the presence of about 1-3 equivalents of base or about 0.01-1 equivalent of copper iodide to give compound (IIbe). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine, pyridine, N,N-dimethylaminopyridine, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IIb), a starting compound (IIb) having a different substituent can be produced by substituent conversion from, as a starting material a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IIb) can be also produced.

### [Production Method C]

Compound (Ic) of the present invention can be obtained by, for example, the method shown by the following scheme or a method analogous thereto and the like.

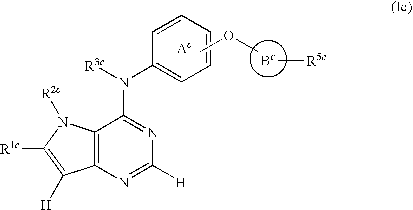

wherein each symbol is as defined above.

Each compound in the following schemes includes salts, and as such salts, for example, those similar to the salts of compound (Ic) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (Ic) of the present invention can be produced, for example, by reacting a compound represented by the formula:

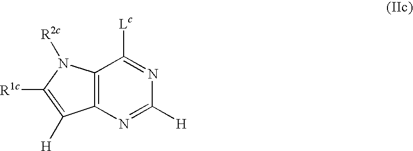

wherein L^{c} is a leaving group, and the other symbols are as defined above,
or a salt thereof and a compound represented by the formula:

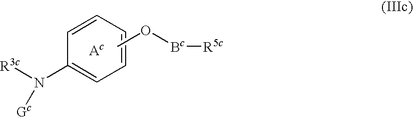

wherein G^{c} is a hydrogen atom or a metal atom, and the other symbols are as defined above,
or a salt thereof.

G^{c} is mainly a hydrogen atom, but it may be an alkali metal such as lithium, sodium, potassium, cesium and the like, or an alkaline earth,metal such as magnesium, calcium and the like.

Compound (IIIc) or a salt thereof is preferably used in an amount of 1-5 equivalents, preferably 1-2 equivalents, relative to compound (IIc) and the reaction is preferably carried out in a solvent. In addition, a base or an ammonium salt may be used in an amount of about 1-10 equivalents, preferably 1-2 equivalents.

In the aforementioned formula, as the leaving group for L^{c}, a halogen atom such as chlorine, bromine, iodine and the like, a group represented by the formula: —S(O)ₖR^{z} wherein k is an integer of 0, 1 or 2, and R^{z} is a lower (C₁₋₄)alkyl group such as methyl, ethyl, propyl and the like, a C₆₋₁₀ aryl group such as phenyl, tolyl and the like, or a group represented by the formula: —OR^{z} wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

A compound within the scope of the present invention can be also produced by applying means known per se to the obtained compound (Ic) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (Ic), which is,a product of the reaction, may be produced as a single compound or as a mixture.

The compound (Ic) of the present invention thus obtained can be subjected to a means known per se, such as solvent extraction, concentration, neutralization, filtration, crystallization, recrystallization, column chromatography, high performance liquid chromatography and the like, whereby the objective compound can be isolated and purified at high purity from a reaction mixture.

As the starting compound (IIIc) of this production method, a commercially available one is used or can be produced by a means known per se.

The starting compound (IIc) of this production method can be produced by, for example, a method shown by the following scheme. Here, compounds (IIca), (IIcb), (IIcc) and (IIcd) are encompassed in compound (IIc).

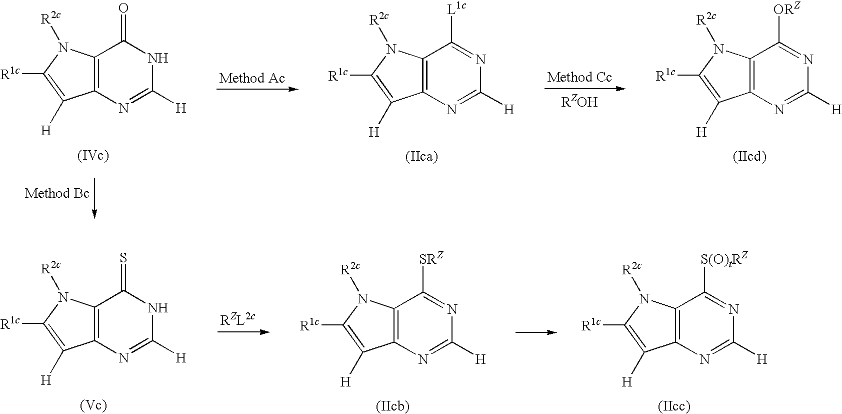

wherein L^{1c} and L^{2c} are halogen atoms, R^{z} is as defined above, and t is an integer of 1 or 2.

As Method Ac, compound (IIca) can be produced by reacting compound (IVc) with a halogenating agent. As Method Bc, compound (IVc) is reacted with a thionating agent to give compound (Vc), which is then reacted with a compound represented by R^{z}L^{2c} in the presence of a base to give compound (IIcb), which is further subjected to an oxidation reaction to give compound (IIcc). As Method Cc, compound (IIca) is reacted with a compound represented by R^{Z}OH in the presence of a base to give compound (IIcd).

As the halogenating agent in Method Ac, for example, about 1-100 equivalents of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1-20 hr, preferably about 1-10 hr.

As the thionating agent used in the production step from compound (IVc) to compound (Vc) in Method Bc, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons, such as benzene, toluene, xylene and the like; ethers such as diethyl ether, tetrahydrofuran, dioxane and the like; and the like can be used. The reaction is carried out at room temperature or under heating, and the reaction time is generally about 1-20 hr, preferably about 1-10 hr.

As R^{z}L^{2c} in the production step from compound (Vc) to compound (IIcb) in Method Bc, for example, about 1-5 equivalents of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

As the oxidizing agent in the production step from compound (IIcb) to compound (IIcc).in Method Bc, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIcd) wherein t=1 is produced, the oxidizing agent is used in about 1-1.5 equivalents relative to compound (IIcb), and when compound (IIcc) wherein t=2 is produced, it is used in about 2-3 equivalents relative to compound (IIcb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

As R^{Z}OH in the production step from compound (IIca) to compound (IIcd) in Method Cc, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and'the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Furthermore, compound (IVc) can be produced by, for example, a method shown by the following formula:

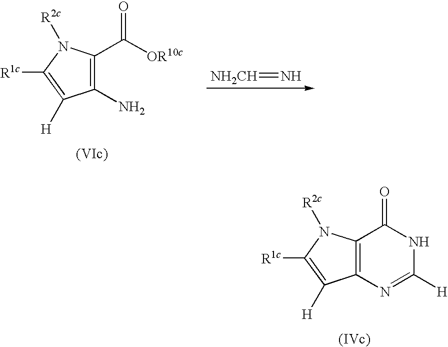

wherein R^{10c} is a C₁₋₄ alkyl group, and other symbols are as defined above.

That is, compound (VIc) is reacted with about 1-4 equivalents of formamidine or a salt thereof to give compound (IVc). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Compound (IIc) can be also produced by, for example, a method shown by the following formula:

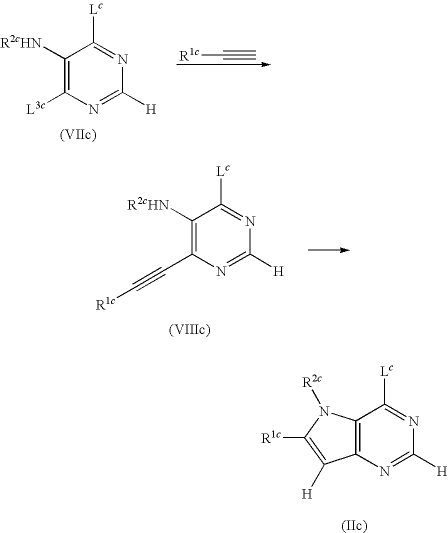

wherein L^{3c} is a halogen atom, and other symbols are as defined above.

For the production step from compound (VIIc) to compound (VIIIc) in this method, a reaction generally known as a Sonogashira reaction or a reaction analogous thereto can be carried out, and generally, compound (VIIIc) can be produced by reacting compound (VIIc) with about 1-3 equivalents of a compound represented by the formula:

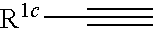

in the presence of a base, about 0.01-1 equivalent of a palladium catalyst and copper iodide. As the base, for example, triethylamine, N-ethyldiisopropylamine, diisopropylamine, pyridine, N,N-dimethylaminopyridine, diazabicycloundecene (DBU), sodium carbonate, potassium carbonate, sodium hydrogencarbonate, potassium hydrogencarbonate and the like can be used. As the palladium catalyst, for example, dichlorobis(triphenylphosphine)palladium(II), palladium on carbon, palladium(II) diacetate, bis(benzonitrile)dichloropalladium(II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried out at room temperature or under heating, and the reaction time is generally about 1-50 hr, preferably about 1-20 hr.

For the production step from compound (VIIIc) to compound (IIc) in this method, a cyclization reaction is generally carried out in the presence of about 1-3 equivalents of base or about 0.01-1 equivalent of copper iodide to give compound (IIc). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine; pyridine, N,N-dimethylaminopyridine, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethokyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IIc), a starting compound (IIc) having a different substituent can be produced by substituent conversion from, as a starting material, a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IIc) can be also produced.

The starting compound (IIc) of this production method can also be produced, for example, by a method using compound (IIc′), as shown by the following scheme:

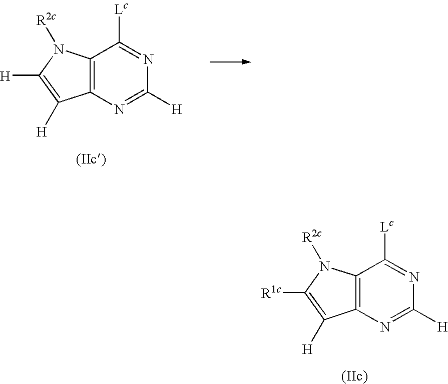

wherein each symbol is as defined above.

In this method, generally, compound (IIc′) is converted to the anion by withdrawing a proton from compound (IIc′) using a base, which is then reacted with a cation having R^{1c} to give compound (IIc). As the base, for example, n-butyllithium, s-butyllithium, t-butyllithium, lithium t-butoxide, lithium diisopropylamide and the like can be used. As a reagent for generating the cation, for example, p-toluenesulfonyl chloride, benzenesulfonyl bromide, p-toluenesulfonyl cyanide, S-(trifluoromethyl) dibenzothiophenium trifluoromethanesulfonate, N,N-dimethylformamide and the like can be used. As the reaction solvent, for example, halogenated hydrocarbons such as dichloromethane, chloroform, carbon tetrachloride, 1,2-dichloroethane and the like; ethers such as diethyl ether, tetrahydrofuran, dioxane, 1,2-dimethoxyethane and the like, a mixed solvent thereof and the like can be used. The aforementioned reaction can be carried out under cooling, preferably about not more than −20° C., and the reaction time is generally about 15 min-50 hr, preferably about 30 min-4 hr.

### [Production Method D]

Compound (Id) of the present invention can be obtained by, for example, the method shown by the following scheme or a method analogous thereto and the like.

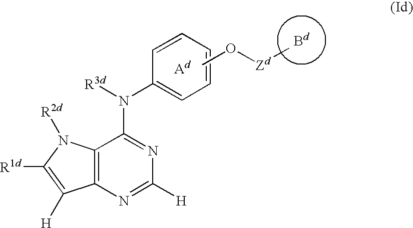

wherein each symbol is as defined above.

Each compound in the following schemes includes salts, and as such salts, for example, those similar to the salts of compound (Id) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (Id) of the present invention can be produced, for example, by reacting a compound represented by the formula:

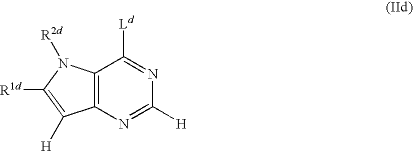

wherein L^{d} is a leaving group, and the other symbols are as defined above,
or a salt thereof and a compound represented by the formula:

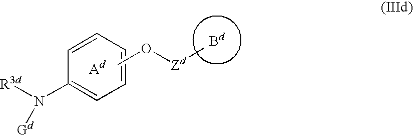

wherein G^{d} is a hydrogen atom or a metal atom, and the other symbols are as defined above,
or a salt thereof.

G^{d} is mainly a hydrogen atom, but it may be an alkali metal such as lithium, sodium, potassium, cesium and the like, or an alkaline earth metal such as magnesium, calcium and the like.

Compound (IIId) or a salt thereof is preferably used in an amount of 1-5 equivalents, preferably 1-2 equivalents, relative to compound (IId) and the reaction is preferably carried out in a solvent. In addition, a base or an ammonium salt may be used in an amount of about 1-10 equivalents, preferably 1-2 equivalents. In the aforementioned formula, as the leaving group for L^{d}, a halogen atom such as chlorine, bromine, iodine and the like, a group represented by the formula: —S(O)ₖR^{z} wherein k is an integer of 0, 1 or 2, and R^{z} is a lower (C₁₋₄)alkyl group such as methyl, ethyl, propyl and the like, a C₆₋₁₀ aryl group such as phenyl, tolyl and the like, or a group represented by the formula: —OR^{z} wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

A compound within the scope of the present invention can be also produced by applying means known per se to the obtained compound (Id) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (Id), which is a product of the reaction, may be produced as a single compound or as a mixture.

The compound (Id) of the present invention thus obtained can be subjected to a means known per se, such as solvent extraction, concentration, neutralization, filtration, crystallization, recrystallization, column chromatography, high performance liquid chromatography and the like, whereby the objective compound can be isolated and purified at high purity from a reaction mixture.

As the starting compound (IIId) of this production method, a commercially available one is used or can be produced by a means known per se.

The starting compound (IId) of this production method can be produced by, for example, a method shown by the following scheme. Here, compounds (IIda), (IIdb), (IIdc) and (IIdd) are encompassed in compound (IId).

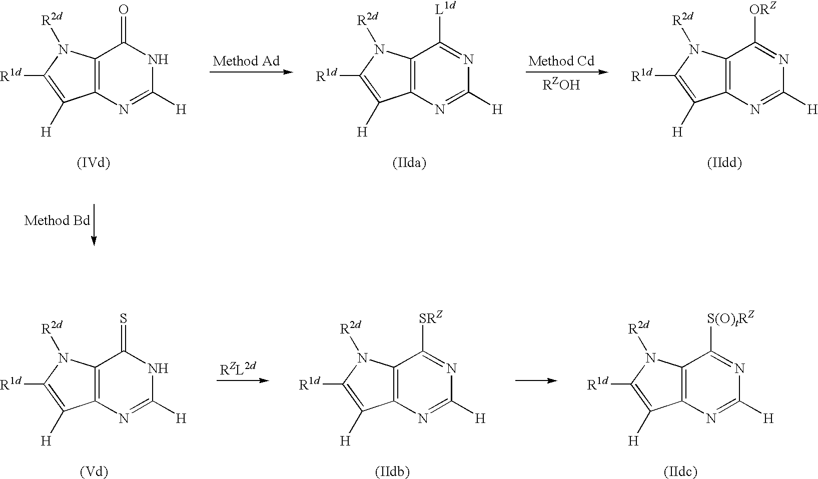

wherein L^{1d} and L^{2d} are halogen atoms, R^{z} is as defined above, and t is an integer of 1 or 2.

As Method Ad, compound (IIda) can be produced by reacting compound (IVd) with a halogenating agent. As Method Bd, compound (IVd) is reacted with a thionating agent to give compound (Vd), which is then reacted with a compound represented by R^{z}L^{2d} in the presence of a base to give compound (IIdb), which is further subjected to an oxidation reaction to give compound (IIdc). As Method Cd, compound (IIda) is reacted with a compound represented by R^{z}OH in the presence of a base to give compound (IIdd).

As the halogenating agent in Method Ad, for example, about 1-100 equivalents of phosphorus pxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1-20 hr, preferably about 1-10 hr.

As the thionating agent used in the production step from compound (IVd) to compound (Vd) in Method Bd, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and the reaction time is generally about 1-20 hr, preferably about 1-10 hr.

As R^{z}L^{2d} in the production step from compound (Vd) to compound (IIdb) in Method Bd, for example, about 1-5 equivalents of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

As the oxidizing agent in the production step from compound (IIdb) to compound (IIdc) in Method Bd, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIdc) wherein t=1 is produced, the oxidizing agent is used in about 1-1.5 equivalents relative to compound (IIdb), and when compound (IIdc) wherein t=2 is produced, it is used in about 2-3 equivalents relative to compound (IIdb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

As R^{z}OH in the production step from compound (IIda) to compound (IIdd) in Method Cd, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 Thr, preferably about 1-10 hr.

Furthermore, compound (IVd) can be produced by, for example, a method shown by the following formula:

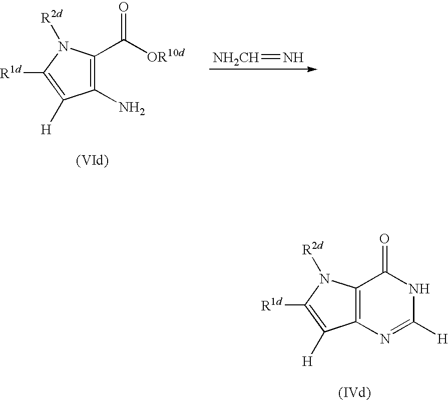

wherein R^{10d} is a C₁₋₄ alkyl group, and other symbols are as defined above.

That is, compound (VId) is reacted with about 1-4 equivalents of formamidine or a salt thereof to give compound (IVd). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Compound (IId) can be also produced by, for example, a method shown by the following formula:

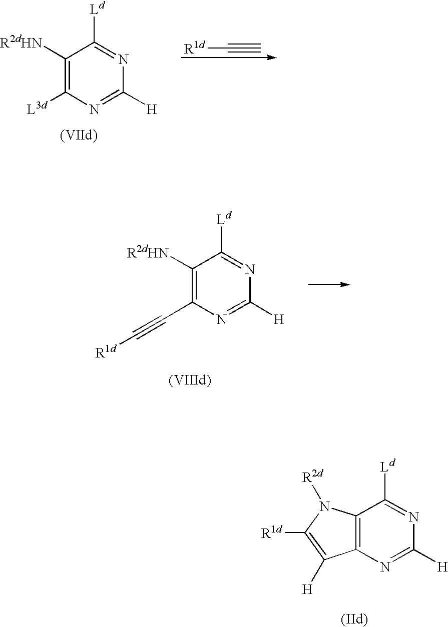

wherein L^{3d} is a halogen atom, and other symbols are as defined above.

For the production step from compound (VIId) to compound (VIIId) in this method, a reaction generally known as a Sonogashira reaction or a reaction analogous thereto can be carried out, and generally, compound (VIIId) can be produced by reacting compound (VIId) with about 1-3 equivalents of a compound represented by the formula:

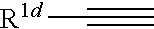

in the presence of a base, about 0.01-1 equivalent of a palladium catalyst and copper iodide. As the base, for example, triethylamine, N-ethyldiisopropylamine, diisopropylamine, pyridine, N,N-dimethylaminopyridine, diazabicycloundecene (DBU), sodium carbonate, potassium carbonate, sodium hydrogencarbonate, potassium hydrogencarbonate and the like can be used. As the palladium catalyst, for example, dichlorobis(triphenylphosphine)palladium(II), palladium on carbon, palladium(II) diacetate, bis(benzonitrile)dichloropalladium(II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried out at room temperature or under heating, and the reaction time is generally about 1-50 hr, preferably about 1-20 hr.

For the production step from compound (VIIId) to compound (IId) in this method, a cyclization reaction is generally carried out in the presence of about 1-3 equivalents of base or about 0.01-1 equivalent of copper iodide to give compound (IId). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine, pyridine, N,N-dimethylaminopyridine, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IId), a starting compound (IId) shaving a different substituent can be produced by substituent conversion from, as a starting material, a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IId) can be also produced.

### [Production Method E]

Compound (Ie) of the present invention can be obtained by, for example, the method shown by the following scheme or a method analogous thereto and the like.

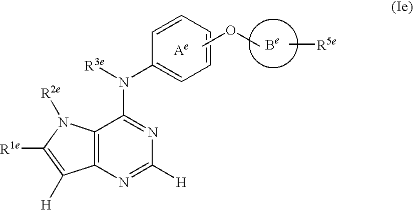

Wherein each symbol is as defined above.

Each compound in the following schemes includes salts, and as such salts, for example, those similar to the salts of compound (Ie) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (Ie) of the present invention can be produced, for example, by reacting a compound represented by the formula:

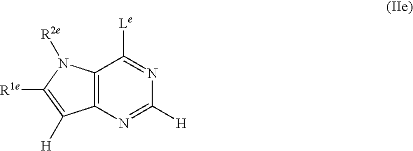

wherein L^{e} is a leaving group, and the other symbols are as defined above,
or a salt thereof and a compound represented by the formula:

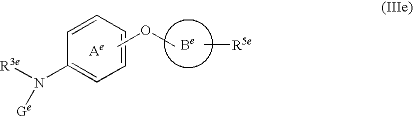

wherein G^{e} is a hydrogen atom or a metal atom, and the other symbols are as defined above,
or a salt thereof.

G^{e} is mainly a hydrogen atom, but it may be an alkali metal such as lithium, sodium, potassium, cesium and the like, or an alkaline earth metal such as magnesium, calcium and the like.

Compound (IIIe) or a salt thereof is preferably used in an amount of 1-5 equivalents, preferably 1-2 equivalents, relative to compound (IIe) and the reaction is preferably carried out in a solvent. In addition, a base or an ammonium salt may be used in an amount of about 1-10 equivalents, preferably 1-2 equivalents.

In the aforementioned formula, as the leaving group for L^{e}, a halogen atom such as chlorine, bromine, iodine and the like, a group represented by the formula: —S(O)ₖR^{z} wherein k is an integer of 0, 1 or 2, and R^{z} is a lower (C₁₋₄)alkyl group such as methyl, ethyl, propyl and the like, a C₆₋₁₀ aryl group such as phenyl, tolyl and the like, or a group represented by the, formula: —OR^{z} wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

A compound within the scope of the present invention can be also produced by applying means known per se to the obtained compound (Ie) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (Ie), which is a product of the reaction, may be produced as a single compound or as a mixture.

The compound (Ie) of the present invention thus obtained can be subjected to a means known per se, such as solvent extraction, concentration, neutralization, filtration, crystallization, recrystallization, column chromatography, high performance liquid chromatography and the like, whereby the objective compound can be isolated and purified at high purity from a reaction mixture.

As the starting compound (IIIe) of this production method, a commercially available one is used or can be produced by a means known per se.

The starting compound (IIe) of this production method can be produced by, for example, a method shown by the following scheme. Here, compounds (IIea), (IIeb), (IIec) and (IIed) are encompassed in compound (IIe).

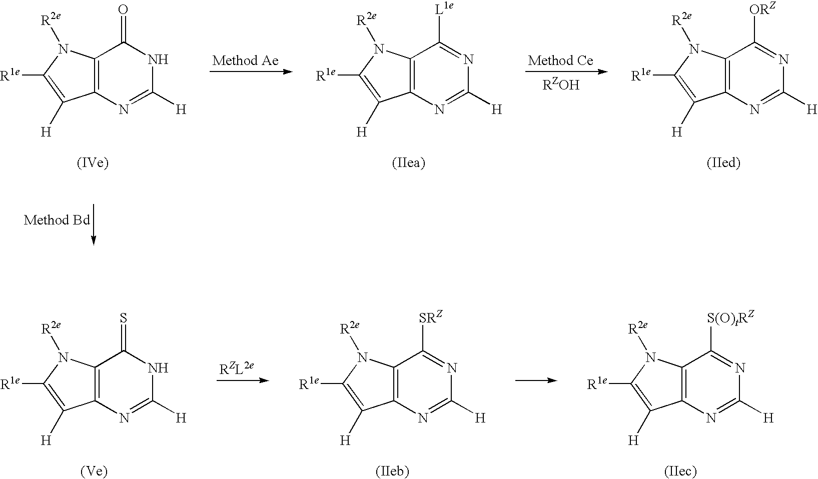

wherein L^{1e} and L^{2e} are halogen atoms, R^{z} is as defined above, and t is an integer of 1 or 2.

As Method Ae, compound (IIea) can be produced by reacting compound (IVe) with a halogenating agent. As Method Be, compound (IVe) is reacted with a thionating agent to give compound (Ve), which is then reacted with a compound represented by R^{z} L^{2e} in the presence of a base to give compound (IIeb), which is further subjected to an oxidation reaction to give compound (IIec). As Method Ce, compound (IIea) is reacted with a compound represented by R^{z}OH in the presence of a base to give compound (IIed).

As the halogenating agent in Method Ae, for example, about 1-100 equivalents of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1-20 hr, preferably about 1-10 hr.

As the thionating agent used in the production step from compound (IVe) to compound (Ve) in Method Be, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and the reaction time is generally about 1-20 hr, preferably about 1-10 hr.

As R^{z}L^{2e} in the production step from compound (Ve) to compound (IIeb) in Method Be, for example, about 1-5 equivalents of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed ;solvent thereof and the like can be used. The reaction is carried out under cooling, at room temperature or, under heating, and the reaction time is generally about 1-20 hr, preferably about 1-10 hr.

As the oxidizing agent in the production step from compound (IIeb) to compound (IIec) in Method Be, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIec) wherein t=1 is produced, the oxidizing agent is used in about 1-1.5 equivalents relative to compound (IIeb), and when compound (IIec) wherein t=2 is produced, it is used in about 2-3 equivalents relative to compound (IIeb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

As R^{z}OH in the production step from compound (IIea) to compound (IIed) in Method Ce, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Furthermore, compound (IVe) can be produced by, for example, a method shown by the following formula:

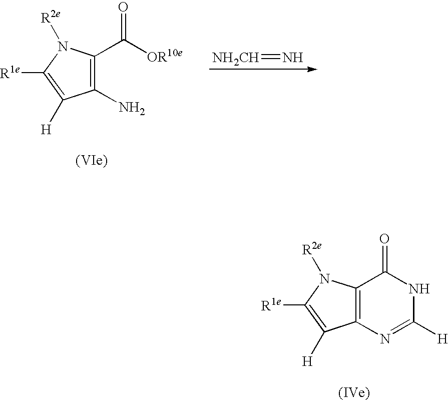

wherein R^{10e} is a C₁₋₄ alkyl group, and other symbols are as defined above.

That is, compound (VIe) is reacted with about 1-4 equivalents of formamidine or a salt thereof to give compound (IVe). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Compound (IIe) can be also produced by, for example, a method shown by the following formula:

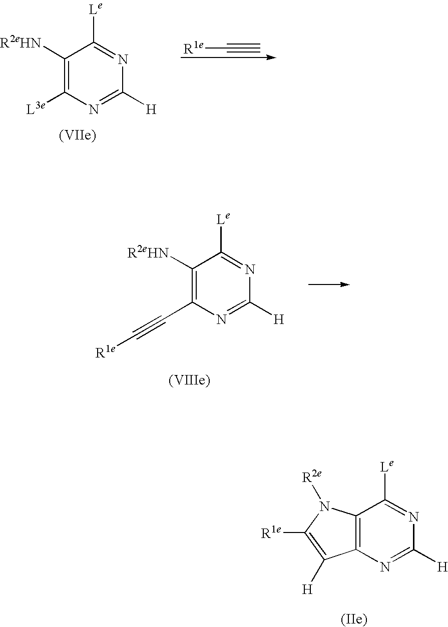

wherein L^{3e} is a halogen atom, and other symbols are as defined above.

For the production step from compound (VIIe) to compound (VIIIe) in this method, a reaction generally known as a Sonogashira reaction or a reaction analogous thereto can be carried out, and generally, compound (VIIIe) can be produced by reacting compound (VIIe) with about 1-3 equivalents of a compound represented by the formula:

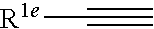

in the presence of a base, about 0.01-1 equivalent of a palladium catalyst and copper iodide. As the base, for example, triethylamine, N-ethyldiisopropylamine, diisopropylamine, pyridine, N,N-dimethylaminopyridine, diazabicycloundecene (DBU), sodium carbonate, potassium carbonate, sodium hydrogencarbonate, potassium hydrogencarbonate and the like can be used. As the palladium catalyst, for example, dichlorobis(triphenylphosphine)palladium(II), palladium on carbon, palladium(II) diacetate, bis(benzonitrile)dichloropalladium(II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried out at room temperature or under heating, and the reaction time is generally about 1-50 hr, preferably about 1-20 hr.

For the production step from compound (VIIIe) to compound (IIe) in this method, a cyclization reaction is generally carried out in the presence of about 1-3 equivalents of base or about 0.01-1 equivalent of copper iodide to give compound (IIe). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine; pyridiner, N,N-dimethylaminopyridine, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IIe), a starting compound (IIe) having a different substituent can be produced by substituent conversion from, as a starting material, a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IIe) can be also produced.

### [Production Method F]

Compound (If) of the present invention can be obtained by, for example, the method shown by the following scheme or a method analogous thereto and the like.

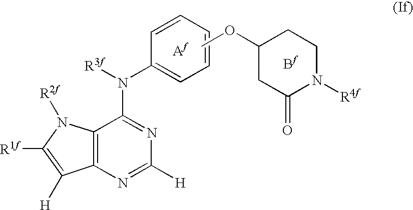

wherein each symbol is as defined above.

Each compound in the following schemes includes salts, and as such salts, for example, those similar to the salts of compound (If) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (If) of the present invention can be produced, for example, by reacting a compound represented by the formula:

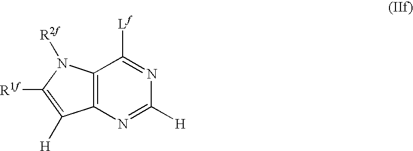

wherein L^{f} is a leaving group, and the other symbols are as defined above,
or a salt thereof and a compound represented by the formula:

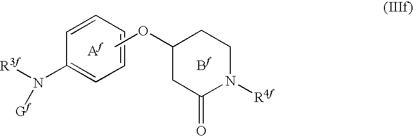

wherein G^{f} is a hydrogen atom or a metal atom, and the other symbols are as defined above,
or a salt thereof.

G^{f} is mainly a hydrogen atom, but it may be an alkali metal such as lithium, sodium, potassium, cesium and the like, or an alkaline earth metal such as magnesium, calcium and the like.

Compound (IIIf) or a salt thereof is preferably used in an amount of 1-5 equivalents, preferably 1-2 equivalents, relative to compound (IIf) and the reaction is preferably carried out in a solvent. In addition, a base or an ammonium salt may be used in an amount of about 1-10 equivalents, preferably 1-2 equivalents.

In the aforementioned formula, as the leaving group for L^{f}, a halogen atom such as chlorine, bromine, iodine and the like, a group represented by the formula: —S(O)ₖR^{z} wherein k is an integer of 0, 1 or 2, and R^{z} is a lower (C₁₋₄)alkyl group such as methyl, ethyl, propyl and the like, a C₆₋₁₀ aryl group such as phenyl, tolyl and the like, or a group represented by the formula: —OR wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

A compound within the scope of the present invention can be also produced by applying means known per se to the obtained compound (If) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (If), which is a product of the reaction, may be produced as a single compound or as a mixture.

The compound (If) of the present invention thus obtained can be subjected to a means known per se, such as solvent extraction, concentration, neutralization, filtration, crystallization, recrystallization, column chromatography, high performance liquid chromatography and the like, whereby the objective compound can be isolated and purified at high purity from a reaction mixture.

As the starting compound (IIIf) of this production method, a commercially available one is used or can be produced by a means known per se.

The starting compound (IIf) of this production method can be produced by, for example, a method shown by the following scheme. Here, compounds (IIfa), (IIfb), (IIfc) and (IIfd) are encompassed in compound (IIf).

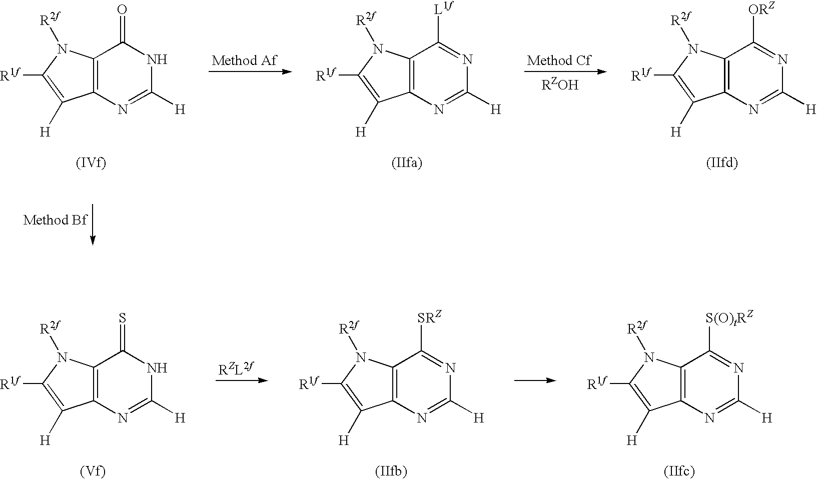

wherein L^{1f} and L^{2f} are halogen atoms, R^{z} is as defined above, and t is an integer of 1 or 2.

As Method Af, compound (IIfa) can be produced by reacting compound (IVf) with a halogenating agent. As Method Bf, compound (IVf) is reacted with a thionating agent to give compound (Vf), which is then reacted with a compound represented by R^{z}L^{2f} in the presence of a base to give compound (IIfb), which is further, subjected to an oxidation reaction to give compound (IIfc). As Method Cf, compound (Ilfa) is reacted with a compound represented by R^{z}OH in the presence of a base to give compound (IIfd).

As the halogenating agent in Method Af, for example, about 1-100 equivalents of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1-20 hr, preferably about 1-10 hr.

As the thionating agent used in the production step from compound (IVf) to compound (Vf) in Method Bf, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and, the reaction time is generally about 1-20 hr, preferably about 1-10 hr.

As R^{z}L^{2f} in the production step from compound (Vf) to compound (IIfb) in Method Bf, for example, about 1-5 equivalents of methyl iodide, benzyl chloride, benzyl bromide and the like can be used, and as the base, for example, sodium hydroxide, potassium hydroxide, sodium carbonate, potasium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

As the oxidizing agent in the production step from compound (IIfb) to compound (IIfc) in Method Bf, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIfc) wherein t=1 is produced, the oxidizing agent is used in about 1-1.5 equivalents relative to compound (IIfb), and when compound (IIfc) wherein t=2 is produced, it is used in about 2-3 equivalents relative to compound (IIfb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

As R^{z}OH in the production step from compound (IIfa) to compound (IIfd) in Method Cf, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Furthermore, compound (IVf) can be produced by, for example, a method shown by the following formula:

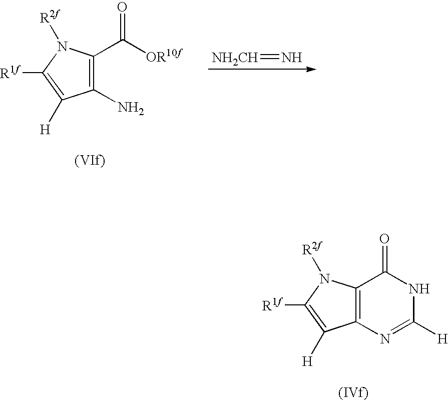

wherein R^{10f} is a C₁₋₄ alkyl group, and other symbols are as defined above.

That is, compound (VIf) is reacted with about 1-4 equivalents of formamidine or a salt thereof to give compound (IVf). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Compound (IIf) can,be also produced by, for example, a method shown by the following formula:

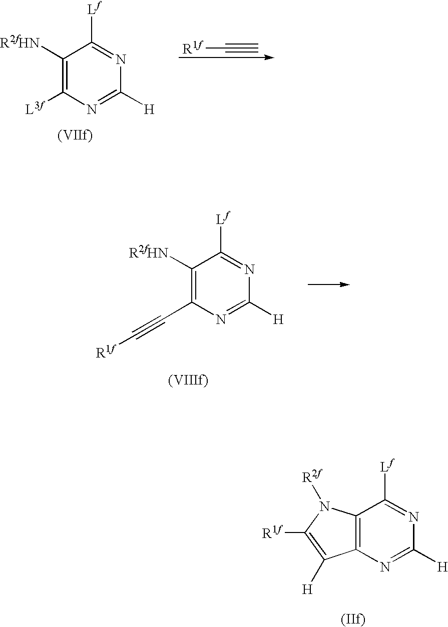

wherein L^{3f} is a halogen atom, and other symbols are as defined above.

For the production step from compound (VIIf) to compound (VIIIf) in this method, a reaction generally known as a Sonogashira reaction or a reaction analogous thereto can be carried out, and generally, compound (VIIIf) can be produced by reacting compound (VIIf) with about 1-3 equivalents of a compound represented by the formula:

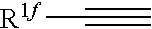

in the presence of a base, about 0.01-1 equivalent of a palladium catalyst and copper iodide. As the base, for example, triethylamine, N-ethyldiisopropylamine, diisopropylamine, pyridine, N,N-dimethylaminopyridine, diazabicycloundecene (DBU), sodium carbonate, potassium carbonate, sodium hydrogencarbonate, potassium hydrogencarbonate and the like can be used. As the palladium catalyst, for example, dichlorobis(triphenylphosphine)palladium(II), palladium on carbon, palladium(II) diacetate, bis(benzonitrile)dichloropalladium(II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried, out at room temperature,or under heating, and the reaction time is generally about 1-50 hr, preferably about 1-20 hr.

For the production step from compound (VIIIf) to compound (IIf) in this method, a cyclization reaction is generally carried out in the presence of about 1-3 equivalents of base or about 0.01-1 equivalent of copper iodide to give compound (IIf). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine, pyridine, N,N-dimethylaminopyridine, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IIf), a starting compound (IIf) having a different substituent can be produced by substituent conversion from, as a starting material, a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IIf) can be also produced.

### [Production Method G]

Compound (Ig) of the present invention can be obtained by, for example, the method shown by the following scheme or a method analogous thereto and the like.

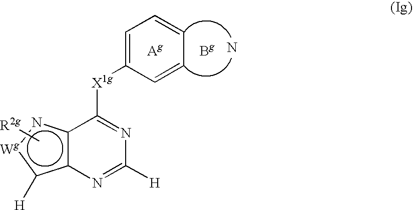

wherein each symbol is as defined above.

Each compound in the following schemes includes salts, and as such salts, for example, those similar to the salts of compound (Ig) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (Ig) of the present invention can be produced, for example, by reacting a compound represented by the formula:

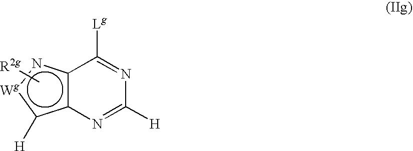

wherein L^{g} is a leaving group, and the other symbols are as defined above,
or a salt thereof and a compound represented by the formula:

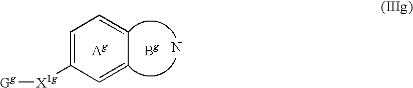

wherein G^{g} is a hydrogen atom or a metal atom, and the other symbols are as defined above,
or a salt thereof.

When X^{1g} is —NR^{3g}—Y^{1g}—, —O— or —S—, G^{g} is mainly a hydrogen atom, but it may be an alkali metal such as lithium, sodium, potassium, cesium and the like, or an alkaline earth metal such as magnesium, calcium and the like.

When X^{1g} is —CHR^{3g}—, G^{g} may be a metal such as lithium, halogenated magnesium, copper, zinc and the like.

Compound (IIIg) or a salt thereof is preferably used in an amount of 1-5 equivalents, preferably 1-2 equivalents, relative to compound (IIg) and the reaction is preferably carried out in a solvent. In addition, a base or an ammonium salt may be used in an amount of about 1-10 equivalents, preferably 1-2 equivalents.

In the aforementioned formula, as the leaving group for L^{g}, a halogen atom such as chlorine, bromine, iodine and the like, a group represented by the formula: —S(O)ₖR^{z} wherein k is an integer of 0, 1 or 2, and R^{z} is a lower (C₁₋₄)alkyl group such as methyl, ethyl, propyl and the like, a C₆₋₁₀ aryl group such as phenyl, tolyl and the like, or a group represented by the formula: —OR^{z} wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

Compound (Ig) wherein X^{1g} is —SO— or —SO₂— can be produced by subjecting compound (Ig) wherein X^{1g} is —S— to an oxidization reaction. As the oxidizing agent in the production step, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (Ig) wherein X^{1g} is —SO— is produced, the oxidizing agent is used in about 1-1.5 equivalents relative to the starting compound, and when compound (Ig) wherein X^{1g} is —SO₂— is produced, it is used in about 2-3 equivalents relative to the starting compound The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

A compound within the scope of the present invention can be also produced by applying means known per se to the obtained compound (Ig) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of Camino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (Ig), which is a product of the reaction, may be produced as a single compound or as a mixture.

The compound (Ig) of the present invention thus obtained can be subjected to a means known per se, such as solvent extraction, concentration, neutralization, filtration, crystallization, recrystallization, column chromatography, high performance liquid chromatography and the like, whereby the objective compound can be isolated and purified at high purity from a reaction mixture.

As the starting compound (IIIg) of this production method, a commercially available one is used or can be produced by a means known per se.

The starting compound (IIg) of this production method can be produced by, for example, a method shown by the following scheme. Here, compounds (IIga), (IIgb), (IIgc), (IIgd) and (IIge) are encompassed in compound (IIg).

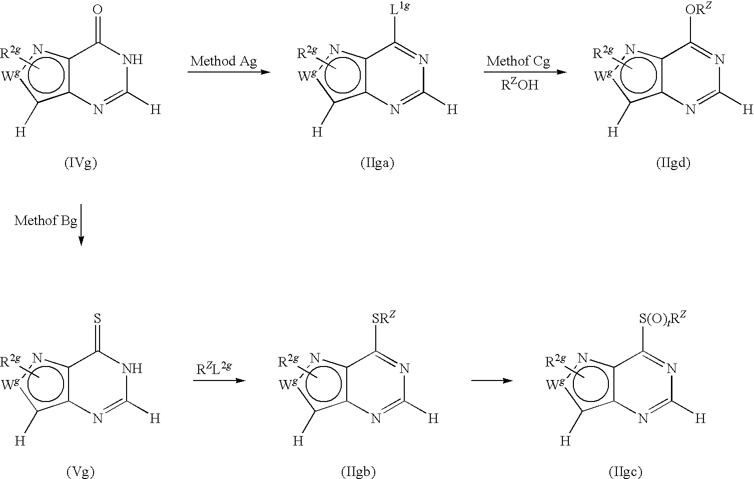

wherein L^{1g} and L^{2g} are halogen atoms, R^{z} is as defined above, and t is an integer of 1 or 2.

As Method Ag, compound (IIga) can be produced by reacting compound (IVg) with a halogenating agent. As Method Bg, compound (IVg) is reacted with a thionating agent to give compound (Vg), which is then reacted with a compound represented by R^{z}L^{2g} in the presence of a base to give compound (IIgb), which is further subjected to an oxidation reaction to give compound (IIgc). As Method Cg, compound (IIga) is reacted with a compound represented by R^{z}OH in the presence of a base to give compound (IIgd).

As the halogenating agent in Method Ag, for example, about 1-100 equivalents of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time generally about 1-20 hr, preferably about 1-10 hr.

As the thionating agent used in the production step from compound (IVg) to compound (Vg) in Method Bg, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and the reaction time is generally about 1-20 hr, preferably about 1-10 hr.

As R^{z}L^{2g} in the production step from compound (Vg) to compound (IIgb) in Method Bg, for example, about 1-5 equivalents of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.,

As the oxidizing agent in the production step from compound (IIgb) to compound (IIgc) in Method Bg, for example, m-chloroperbenzoic acid, hydrogen peroxide, peracetic acid, t-butyl hydroperoxide, potassium peroxysulfate, potassium permanganate; sodium perborate, sodium periodate, sodium hypochlorite, halogen and the like can be used. When compound (IIgc) wherein t=1 is produced, the oxidizing agent is used in about 1-1.5 equivalents relative to compound (IIgb), and when compound (IIgc) wherein t=2 is produced, it is used in about 2-3 equivalents relative to compound (IIgb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

As R^{z}OH in the production step from compound (IIga) to compound (IIgd) in Method Cg, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-diffethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Furthermore, compound (IVg) can be produced by, for example, a method shown by the following formula:

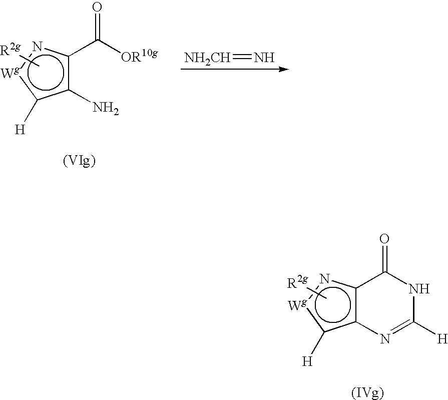

wherein R^{10g} is a C₁₋₄ alkyl group, and other symbols are as defined above:

That is, compound (VIg) is reacted with about 1-4 equivalents of formamidine or a salt thereof to give compound (IVg). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

When W^{g} is C(R^{1g}), compound (IIge) can be also produced by, for example, a method shown by the following formula:

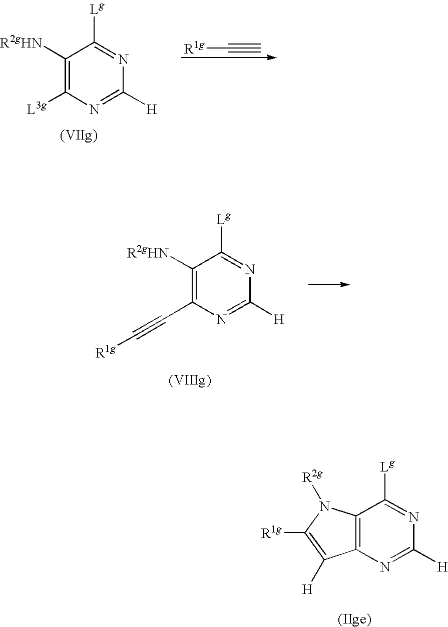

wherein L^{g} is a halogen atom, and other symbols are as defined above.

For the production step from compound (VIIg) to compound (VIIIg) in this method, a reaction generally known as a Sonogashira reaction or a reaction analogous thereto can be carried out, and generally, compound. (VIIIg) can be produced by reacting compound (VIIg) with about 1-3 equivalents of a compound represented by the formula:

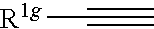

in the presence of a base, about 0.01-1 equivalent of a palladium catalyst and copper iodide. As the base, for example, triethylamine, N-ethyldiisopropylamine, diisopropylamine, pyridine, N,N-dimethylahinopyridine, diazabicycloundecene (DBU), sodium carbonate, potassium carbonate, sodium hydrogencarbonate, potassium hydrogencarbonate and the like can be used. As the palladium catalyst, for example, dichlorobis(triphenylphosphine)palladium(II), palladium on carbon, palladium(II) diacetate, bis(benzonitrile)dichloropalladium(II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried out at room temperature or under heating, and the reaction time is generally about 1-50 hr, preferably about 1-20 hr.

For the production step from compound (VIIIg) to compound (IIge) in this method, a cyclization reaction is generally carried out in the presence of about 1-3 equivalents of base or about 0.01-1 equivalent of copper iodide to give compound (IIge). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine, pyridine, N,N-dimethylaminopyridine, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IIg), a starting compound (IIg) having a different substituent can be produced by substituent conversion from, as a starting material, a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, Oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IIg) can be also produced.

### [Production Method H]

Compound (Ih) of the present invention can be obtained by, for example, the method shown by the following scheme or a method analogous thereto and the like.

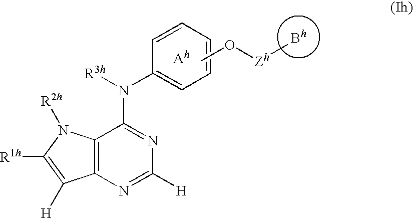

wherein each symbol is as defined above.

Each compound in the following schemes includes salts, and as such salts, for example, those similar to the salts of compound (Ih) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (Ih) of the present invention can be produced, for example, by reacting a compound represented by the formula:

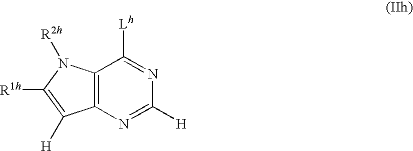

wherein L^{h} is a leaving group, and the other symbols are as defined above,
or a salt thereof and a compound represented by the formula:

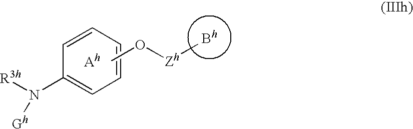

wherein G^{h} is a hydrogen atom or a metal atom, and the other symbols are as defined above,
or a salt thereof.

G^{h} is mainly a hydrogen atom, but it may be an alkali metal such as lithium, sodium, potassium, cesium and the like, or an alkaline earth metal such as magnesium, calcium and the like.

Compound (IIIh) or a salt thereof is preferably used in an amount of 1-5 equivalents, preferably 1-2 equivalents, relative to compound (IIh) and the reaction is preferably carried out in a solvent. In addition, a base or an ammonium salt may be used in an amount of about 1-10 equivalents, preferably 1-2 equivalents.

In the aforementioned formula, as the leaving group for L^{h}, a halogen atom such as chlorine, bromine, iodine and the like, a group represented by the formula: —S(O)ₖFe wherein k is an integer of 0, 1 or 2, and R^{z} is a lower (C₁₋₄) alkyl group such as methyl, ethyl, propyl and the like, a C₆₋₁₀ aryl group such as phenyl, tolyl and the like, or a group represented by the formula: —OR^{z} wherein R^{z} is as defined above, and the like can be used.

As the solvent in the aforementioned reaction, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40-200° C., preferably about 40-160° C.), and the reaction time is generally about 1-30 hr, preferably about 1-20 hr, more preferably about 1-10 hr.

A compound within the scope of the present invention can be also produced by applying means known per se to the obtained compound (Ih) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

The compound (Ih), which is a product of the reaction, may be produced as a single compound or as a mixture.

The compound (Ih) of the present invention thus obtained can be subjected to a means known per se, such as solvent extraction, concentration, neutralization, filtration, crystallization, recrystallization, column chromatography, high performance liquid chromatography and the like, whereby the objective compound can be isolated and purified at high purity from a reaction mixture.

As the starting compound (IIIh) of this production method, a commercially available one is used or can be produced by a means known per se.

The starting compound (IIh) of this production method can be produced by, for example, a method shown by the following scheme. Here, compounds (IIha), (IIhb), (IIhc) and (IIhd) are encompassed in compound (IIh).

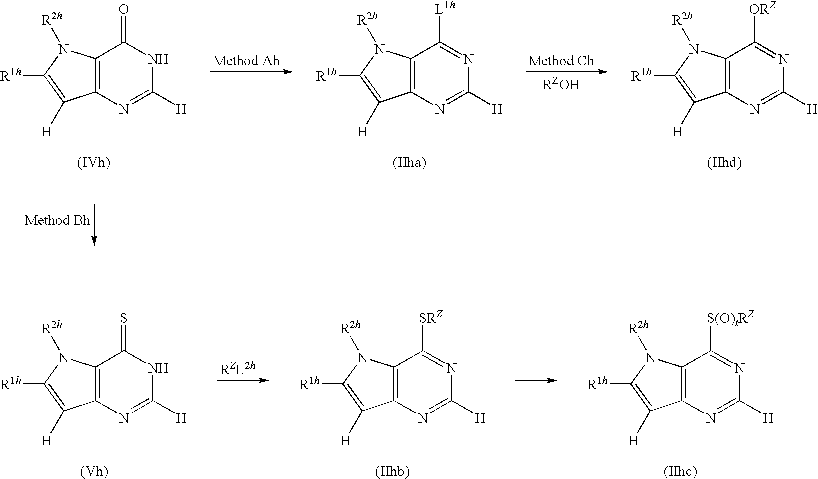

wherein L^{th} and L^{2h} are halogen atoms, R^{z} is as defined above, and t is an integer of 1 or 2.

As Method Ah, compound (IIha) can be produced by reacting compound (IVh) with a halogenating agent. As Method Bh, compound (IVh) is reacted with a thionating agent to give compound (Vh), which is then reacted with a compound represented by R^{z}L^{2h} in the presence of a base to give compound (IIhb), which is further subjected to an oxidation reaction to give compound (IIhc). As Method Ch, compound (IIha) is reacted with a compound represented by R^{z}OH in the presence of a base to give compound (IIhd).

As the halogenating agent in Method Ah, for example, about 1-100 equivalents of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1-20 hr, preferably about 1-10 hr.

As the thionating agent used in the production, step from compound (IVh) to compound (Vh) in Method Bh, for example, about 1-5 equivalents of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and the reaction time is generally about 1-20 hr, preferably about 1-10 hr.

As R^{z}L^{2h} in the production step from compound (Vh) to compound (IIhb) in Method Bh, for example, about 1-5 equivalents of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

As the oxidizing agent in the production step from compound (IIhb) to compound (IIhc) in Method Bh, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIhc) wherein t=1 is produced, the oxidizing agent is used in about 1-1.5 equivalents relative to compound (IIhb), and when compound (IIhc) wherein t=2 is produced, it is used in about 2-3 equivalents relative to compound (IIhb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1-20 hr, preferably about 1-10 hr.

As R^{z}OH in the production step from compound (IIha) to compound (IIhd) in Method Ch, for example, about 1-10 equivalents of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Furthermore, compound (IVh) can be produced by, for example, a method shown by the following formula:

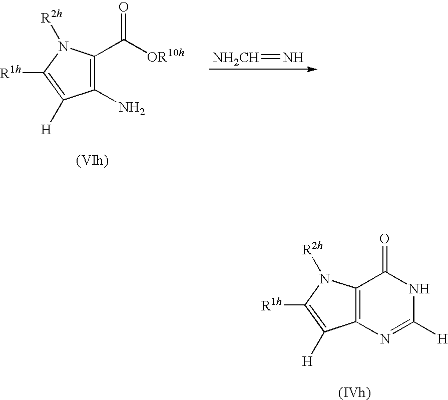

wherein R^{10h} is a C₁₋₄ alkyl group, and other symbols are as defined above.

That is, compound (VIh) is reacted with about 1-4 equivalents of formamidine or a salt thereof to give compound (IVh). As the reaction solvent, for example, alcohols such as methanol, ethanol, isopropanol, t-butanol and the like; halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1-20 hr, preferably about 1-10 hr.

Compound (IIh) can be also produced by, for example, a method shown by the following formula:

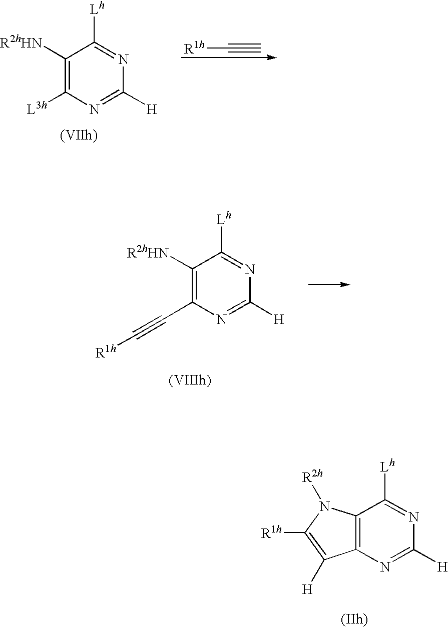

wherein L^{3h} is a halogen atom, and other symbols are as defined above.

For the production step from compound (VIIh) to compound (VIIIh) in this method, a reaction generally known as a Sonogashira reaction or a reaction analogous thereto can be carried out, and generally, compound (VIIIh) can be produced by reacting compound (VIIh) with about 1-3 equivalents of a compound represented by the formula:

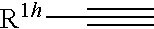

in the presence of a base, about 0.01-1 equivalent of a palladium catalyst and copper iodide. As the base, for example, triethylamine, N-ethyldiisopropylamine, diisopropylamine, pyridine, N,N-dimethylaminopyridine, diazabicycloundecene (DBU), sodium carbonate, potassium carbonate, sodium hydrogencarbonate, potassium hydrogencarbonate and the like can be used. As the palladium catalyt, for example, dichlorobis(triphenylphosphine)palladium(II), palladium on carbon, palladium(II) diacetate, bis(benzonitrile)dichloropalladium (II) and the like can be used. This reaction may be carried out in the co-presence of a tertiary phosphine compound such as triphenylphosphine, tributylphosphine and the like as a ligand. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such,as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. This reaction is carried out at room temperature or under heating, and the reaction time is generally about 1-50 hr, preferably about 1-20 hr.

For the production step from compound (VIIIh) to compound (IIh) in this method, a cyclization reaction is generally carried out in the presence of about 1-3 equivalents of base or about 0.01-1 equivalent of copper iodide to give compound (IIh). As the base, for example, potassium t-butoxide, sodium t-butoxide, cesium t-butoxide, sodium ethoxide, potassium hydride, sodium hydride, cesium hydroxide, sodium hydroxide, potassium hydroxide, sodium carbonate, potassium carbonate, sodium hydrogencarbonate, potassium hydrogencarbonate, triethylamine, N-ethyldiisopropylamine, diisopropylamine, pyridine, N,N-dimethylaminopyridine, diazabicycloundecene (DBU) and the like can be used: As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane, 1,2-dimethoxyethane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out at low temperature, at room temperature or under heating, and the reaction time is generally about 1-50 hr, preferably about 1-20 hr.

Depending on the kind of the substituent of starting compound (IIh), a starting compound (IIh) having a different substituent can be produced by substituent conversion from, as a starting material, a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (IIh) can be also produced.

The starting compound (IIh) of this production method can also be produced, for example, by a method using compound (IIh′), as shown by the following scheme:

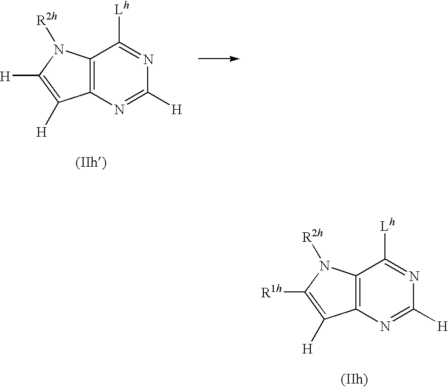

wherein each symbol is as defined above.

In this method, generally, compound (IIh′) is converted to the anion by withdrawing a proton from compound (IIh′) using a base, which is then reacted with a cation having R^{1h} to give compound (IIh). As the base, for example, n-butyllithium, s-butyllithium, t-butyllithium, lithium t-butoxide, lithium diisopropylamide and the like can be used. As a reagent for generating the cation, for example, p-toluenesulfonyl chloride, benzenesulfonyl bromide, p-toluenesulfonyl cyanide, S-(trifluoromethyl) dibenzothiophenium trifluoromethanesulfonate, N,N-dimethylformamide and the like can be used. As the reaction solvent, for example, halogenated hydrocarbons such as dichloromethane, chloroform, carbon tetrachloride, 1,2-dichloroethane and the like; ethers such as diethyl ether, tetrahydrofuran, dioxane, 1,2-dimethoxyethane and the like, a mixed solvent thereof and the like can be used. The aforementioned reaction can be carried out under cooling, preferably about not more than −20° C., and the reaction time is generally about 15 min-50 hr, preferably about 30 min-4 hr.

Thus-obtained compounds (Ia)-(Ih) can be isolated and purified by a separation means known per se, such as concentration, concentration under reduced pressure, solvent extraction, crystallization, recrystallization, phase transfer, chromatography and the like.

If compounds (Ia)-(Ih) are obtained as a free form, it can be converted into a desired salt by a method known per se or a modification thereof; conversely, if compounds (Ia)-(Ih) are obtained as a salt, it can be converted into a free form or another desired salt by a method known per se or a modification thereof.

When compounds (Ia)-(Ih) have isomers such as optical isomer, stereoisomer, positional isomer, rotational isomer and the like, and any isomers and mixtures are encompassed in the compound (Ia)-(Ih). For example, when compounds (Ia)-(Ih) have an optical isomer, an optical isomer separated from a racemate is also encompassed in the compound (Ia)-(Ih). These isomers can be obtained as independent products by a synthesis means or a separation means (concentration, solvent extraction, column chromatography, recrystallization and the like) known per se.

The compounds (Ia)-(Ih) may be a crystal, and both a single crystal and crystal mixtures are encompassed in the compound (Ia)-(Ih). Crystals can be produced by crystallization according to crystallization methods known per se.

The compounds (Ia)-(Ih) may be a solvate (e.g., hydrate etc.) or a non-solvate, both of which are encompassed in the compound (Ia)-(Ih).

A compound labeled with an isotope (e.g., ³H, ¹⁴C, ³⁵S, ¹²⁵I and the like) is also encompassed in the compound (Ia)-(Ih).

A prodrug of the compounds (Ia)-(Ih) or salts thereof (hereinafter referred to as compound (Ia)-(Ih)) means a compound which is converted to the compounds (Ia)-(Ih) with a reaction due to an enzyme, an gastric acid, etc. under the physiological, condition in the living body, that is, a compound which is converted to the compounds (Ia)-(Ih) with oxidation, reduction, hydrolysis, etc. due to an enzyme; a compound which is converted to the compounds (Ia)-(Ih) by hydrolysis etc. due to gastric acid, etc. A prodrug for compounds (Ia)-(Ih) may be a compound obtained by subjecting an amino group in compounds (Ia)-(Ih) to an acylation, alkylation or phosphorylation (e.g., a compound obtained by subjecting an amino group in compounds (Ia)-(Ih) to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation or tert-butylation); a compound obtained by subjecting a hydroxy group in compounds (Ia)-(Ih) to an acylation, alkylation, phosphorylation or boration (e.g., a compound obtained by subjecting an hydroxy group in compounds (Ia)-(Ih) to an acetylation, palmitoylation, propanoylation, pivaloylation, succinylation, fumarylation, alanylation or dimethylaminomethylcarbonylation); a compound obtained by subjecting a carboxyl group in compounds (Ia)-(Ih) to an esterification or amidation (e.g., a compound obtained by subjecting a carboxyl group in compounds (Ia)-(Ih) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. Any one of these compounds can be produced from compounds (Ia)-(Ih) by a method known per se.

A prodrug for compounds (Ia)-(Ih) may also be one which is converted into compounds (Ia)-(Ih) under a physiological condition, such as those described in IYAKUHIN no KAIHATSU (Development of Pharmaceuticals), Vol. 7, Design of Molecules, p.163-198, Published by HIROKAWA SHOTEN (1990).

The compounds (Ia)-(Ih) of the present invention, or a salt thereof or a prodrug thereof (hereinafter referred to as the compound of the present invention) possess tyrosine kinase-inhibiting activity and can be used for the prophylaxis or treatment of tyrosine kinase-dependent diseases in mammals. Tyrosine kinase-dependent diseases include diseases characterized by increased cell proliferation due to abnormal tyrosine kinase enzyme activity.

Particularly, the compound of the present invention specifically inhibits HER2 kinase and/or EGFR kinase and is therefore also useful as a therapeutic agent for suppressing the growth of HER2 and/or EGFR kinase-expressing cancer. Also, the compound of the present invention is useful as a preventive agent for preventing hormone-dependent cancer and the transition of hormone-dependent cancer to hormone-independent cancer.

In addition, the compound of the present invention is useful as a pharmaceutical agent because it shows low toxicity (e.g., acute toxicity, chronic toxicity, genetic toxicity, reproductive toxicity, cardiotoxicity, drug interaction, carcinogenicity and the like), high water solubility, and is superior in stability, pharmacokinetics (absorption, distribution, metabolism, excretion and the like) and efficacy expression.

Accordingly, the compound of the present invention can be used as a safe agent for the prophylaxis or treatment of diseases due to abnormal cell proliferation such as various cancers (particularly, breast cancer (e.g., invasive ductal carcinoma, ductal cancer in situ, inflammatory breast cancer etc.), prostate cancer (e.g., hormone-dependent prostate cancer, non-hormone dependent prostate cancer etc.), pancreatic cancer (e.g., pencreatic duct cancer etc.), gastric cancer (e.g., papillary adenocarcinoma, mucinous adenocarcinoma, adenosquamous carcinoma etc.), lung cancer (e.g., non-small cell lung cancer, small cell lung cancer, malignant mesothelioma etc.), colorectal cancer (e.g., familial colorectal cancer, hereditary nonpolyposis colorectal cancer, gastrointestinal stromal tumor etc.), colon cancer (e.g., gastrointestinal stromal tumor etc.), rectal cancer (e.g., gastrointestinal stromal tumor etc.), esophagus cancer, duodenal cancer, cancer of the tongue, cancer of pharynx (e.g., nasopharyngeal carcinoma, oropharyngeal cancer, hypopharyngeal cancer etc.), salivary gland cancer, brain tumor (e.g., pineal astrocytoma, pilocytic astrocytoma, diffuse astrocytoma, anaplastic astrocytoma etc.), neurinoma, non-small cell lung cancer, small cell lung cancer, liver cancer (e.g., primary liver cancer, Extrahepatic Bile Duct Cancer etc.), kidney cancer (e.g., renal cell carcinoma, renal pelvis and ureter, transitional cell cancer etc.), cancer of the bile duct, cancer of the uterine body, endometrial carcinoma, cancer of the uterine cervix, ovarian cancer (e.g., ovarian epithelial, extragonadal germ cell tumor, ovarian germ cell tumor, ovarian low malignant potential tumor etc.), urinary bladder cancer, urethral cancer, skin cancer (e.g., ocular melanoma, Merkel cell carcinoma etc.), hemangioma, malignant lymphoma, malignant melanoma, thyroid cancer (e.g., medullary thyroid carcinoma etc.), parathyroid cancer, nasal cavity cancer, paranasal sinus cancer, bone tumors (e.g., osteosarcoma, Ewing's tumor, uterus sarcoma, soft tissue sarcoma etc.), vascular fibroma, retinoblastoma, penile cancer, solid cancer in childhood, Kaposi s sarcoma, Kaposi's sarcoma derived from AIDS, maxillary tumor, fibrous histiocytoma, leiomyosarcoma, rhabdomyosarcoma, leukemia (e.g., acute myeloid leukemia, acute lymphoblastic leukemia etc.) etc.), atherosclerosis, angiogenesis (e.g., angiogenesis associated with growth of solid cancer and sarcoma, angiogenesis associated with tumor metastasis, angiogenesis associated with diabetic retinopathy, etc.), and viral diseases (HIV infection etc:).

Tyrosine kinase-dependent diseases further include cardiovascular diseases associated with abnormal tyrosine kinase enzyme activity. The compound of the present invention can therefore be used as an agent for prophylaxis or treatment of cardiovascular diseases such as restenosis.

The compound of the present invention is useful as an anticancer agent for the prophylaxis or treatment of cancer, especially breast cancer, ovarian cancer, colorectal cancer, gastric cancer, esophagus cancer, prostate cancer, lung cancer, pancreatic cancer, kidney cancer and the like.

The compound of the present invention shows low toxicity and can be used as a pharmaceutical agent as it is, or as a pharmaceutical composition in admixture with a commonly known pharmaceutically acceptable carrier etc. in mammals (e.g., humans, horses, bovines, dogs, cats, rats, mice, rabbits, pigs, monkeys and the like).

In addition to the compound of the present invention, said pharmaceutical composition may contain other active ingredients, e.g., the following hormonal therapeutic agents, anticancer agent (e.g., chemotherapeutic agents, immunotherapeutic agents, or pharmaceutical agents inhibiting the action of cell growth factors or cell growth factor receptors), and the like.

As a pharmaceutical agent for-mammals such as humans, the compound of the present invention can be administered orally in the form of, for example, tablets, capsules (including soft capsules and microcapsules), powders, granules and the like, or parenterally in the form of injections, suppositories, pellets and the like. Examples of the “parenteral administration route” include intravenous, intramuscular, subcutaneous, intra-tissue, intranasal, intradermal, instillation, intracerebral, intrarectal, intravaginal, intraperitoneal, intratumoral, juxtaposition of tumor and administration directly to the lesion.

The dose of the compound of the present invention varies depending on the route of administration, symptoms, etc. For example, when it is administered orally as an anticancer agent to a patient (body weight 40 to 80 kg) with breast cancer or prostate cancer, its dose is, for example, 0.5 to 100 mg/kg body weight per day, preferably 1 to 50 mg/kg body weight per day, and more preferably 1 or 25 mg/kg body weight per day. This amount may be administered once or in 2 to 3 divided portions daily.

The compound of the present invention can be safely administered orally or parenterally (e.g., topical, rectal, intravenous administrations etc.) as a single agent, or a pharmaceutical composition containing a pharmacologically acceptable carrier according to a conventional method (e.g., a method described in the Japanese Pharmacopoeia etc.), such as tablet (including sugar-coated tablet, film-coated tablet), powder, granule, capsule, liquid, emulsion, suspension, injection, suppoSitory, sustained release preparation, plaster and the like.

And (1) administering an effective amount of a compound of the present invention and (2) a combination of 1 to 3 selected from the group consisting of (i) administering an effective amount of other anticancer agents, (ii) administering an effective amount of hormonal therapeutic agents and (iii) non-drug therapy can prevent and/or treat cancer more effectively. As the non-drug therapy, for example, surgery, radiotherapy, gene therapy, thermotherapy, cryotherapy, laser cauterization and the like are exemplified and two or more of these may be combined.

For example, the compound of the present invention can be administered to the same subject simultaneously, with hormonal therapeutic agents, anticancer agents (e.g., chemotherapeutic agents, immunotherapeutic agents, or pharmaceutical agents inhibiting the action of cell growth factors or cell growth factor receptors) (hereafter, these are referred to as a concomitant drug).

Although the compound of the present invention exhibits excellent anticancer action even when used as a simple agent, its effect can be enhanced by using it in combination with one or more of the concomitant drug(s) mentioned above (multi-agent co-administration).

As examples of said “hormonal therapeutic agents”, there may be mentioned fosfestrol, diethylstylbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, Tadenan, mepartricin, raloxifene, ormeloxifene, levormeloxifene, anti-estrogens (e.g., tamoxifen citrate, toremifene citrate, and the like), ER down regulator (e.g., fulvestrant, and the like), human menopausal gonadotrophin, follicle stimulating hormone, pill preparations, mepitiostane, testrolactone, aminoglutethimide, LH-RH agonists (e.g., goserelin acetate, buserelin, leuprorelin, and the like), droloxifene, epitiostanol, ethinylestradiol sulfonate, aromatase inhibitors (e.g., fadrozole hydrochloride, anastrozole, retrozole, exemestane, vorozole, formestane, and the like), anti-androgens (e.g., flutamide, bicartamide, nilutamide, and the like), 5α-reductase inhibitors (e.g., finasteride, dutasteride, epristeride, and the like), adrenocorticohormone drugs (e.g., dexamethasone, prednisolone, betamethasone, triamcinolone, and the like), androgen synthesis inhibitors (e.g., abiraterone, and the like), retinoid and drugs that retard retinoid metabolism (e.g., liarozole, and the like), etc and LH-RH agonists (e.g., goserelin acetate, buserelin, leuprorelin) are preferable.

As examples of said “chemotherapeutic agents”, there may be mentioned alkylating agents, antimetabolites, anticancer antibiotics, plant-derived anticancer agents, and the like.

As examples of “alkylating agents”, there may be mentioned nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, sodium estramustine phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin, and the like.

As examples of “antimetabolites”, there may be mentioned mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emmitefur, and the like), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine, and the like.

As examples of “anticancer antibiotics”, there may be mentioned actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, and the like.

As examples of “plant-derived anticancer agents”, there may be mentioned etoposide, etoposide phosphate, vinblastine sulfate, vincristine sulfate, vindesine sulfate, teniposide, paclitaxel (Taxol (trade mark)), docetaxel, vinorelbine, and the like.

As examples of said “immunotherapeutic agents (BRM)”, there may be mentioned picibanil, krestin, sizofiran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, and the like.

The “growth factor” in said “pharmaceutical agents inhibiting the action of cell growth factors or cell growth factor receptors”, there may be mentioned any substances that promote cell proliferation, which are normally peptides having a molecular weight of not more than 20,000 that are capable of exhibiting their activity at low concentrations by binding to a receptor, including (1) EGF (epidermal growth factor) or substances possessing substantially the same activity as it [e.g., EGF, heregulin, and the like], (2) insulin or substances possessing substantially the same activity as it [e.g., insulin, IGF (insulin-like growth factor)-1, IGF-2, and the like], (3) FGF (fibroblast growth factor) or substances possessing substantially the same activity as it [e.g., acidic FGF, basic FGF, KGF (keratinocyte growth factor), FGF-10, and the like], (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor (3), HGF (hepatocyte growth factor), VEGF (vascular endothelial growth factor), and the like], and the like.

As examples of said “growth factor receptors”, there may be mentioned any receptors capable of binding to the aforementioned growth factors, including EGF receptor, heregulin receptor (HER2), insulin receptor, IGF receptor, FGF receptor-1 or FGF receptor-2, and the like.

As examples of said “pharmaceutical agents inhibiting the action of cell growth factor”, there may be mentioned HER2 antibody (trastuzumab (Herceptin (trade mark))), imatinib mesilate, ZD1839 or EGFR antibody (cetuximab (Erbitux (trade mark)) etc.), antibody against VEGF (e.g., bevacizumab (Avastin (trade mark))), VEGFR antibody, VEGFR inhibitor, EGFR inhibitor (gefitinib (Iressa (trade mark)), erlotinib (Tarceva, (trade mark)) etc.) and the like.

In addition to the aforementioned drugs, L-asparaginase, aceglatone, procarbazine hydrochloride, protoporphyrin-cobalt complex salt, mercuric hematoporphyrin-sodium, topoisomerase I inhibitors (e.g., irinotecan, topotecan, and the like), topoisomerase II inhibitors (e.g., sobuzoxane, and the like), differentiation inducers (e.g., retinoid, vitamin D, and the like), angiogenesis inhibitors (e.g., thalidomide, SU11248, and the like), α-blockers (e.g., tamsulosin hydrochloride, naftopidil, urapidil, alfuzosin, terazosin, prazosin, silodosin, and the like), serine/threonine kinase inhibitor, endothelin receptor antagonist (e.g., atrasentan, and the like), proteasome inhibitor (e.g., bortezomib, and the like), Hsp 90 inhibitor (e.g., 17-AAG, and the like), spironolactone, minoxidil, 11α-hydroxyprogesterone, bone resorption inhibiting/metastasis suppressing agent (e.g., zoledronic acid, alendronic acid, pamidronic acid, etidronic acid, ibandronic acid, clodronic acid) and the like can be used.

Of those mentioned above, as the concomitant drug, LH-RH agonist (e.g., goserelin acetate, buserelin, leuprorelin, and the like), HER2 antibody (trastuzumab (Herceptin (trade mark))), EGFR antibody (cetuximab (Erbitux) (trade mark) etc.), EGFR inhibitor (erlotinib (Tarceva) (trade mark), gefitinib (Iressa (trade mark)) etc.), VEGFR inhibitor or chemotherapeutic agent (paclitaxel(Taxol (trade mark) etc.) are preferable.

Particularly, trastuzumab (Herceptin (trade mark)), cetuximab (Erbitux (trade mark)), erlotinib (Tarceva) (trade mark)), gefitinib (Iressa (trade mark)), paclitaxel (Taxol(trade mark)) and the like preferable.

In combination of the compound of the present invention and the concomitant drug, the administration time of the compound of the present invention and the concomitant drug is not restricted, and the compound of the present invention and the concomitant drug can be administered to the administration subject simultaneously, or may be administered at different times. The dosage of the concomitant drug may be determined according to the administration amount clinically used, and can be appropriately selected depending on the administration subject, administration route, disease, combination and the like.

The administration mode of the compound of the present invention and the concomitant drug is not particularly restricted, and it is sufficient that the compound of the present invention and the concomitant drug are combined in administration. Examples of such administration mode include the following methods:

(1) The compound of the present invention and the concomitant drug are simultaneously produced to give a single preparation which is administered. (2) The compound of the present invention and the concomitant drug are separately produced to give two kinds of preparations which are administered simultaneously by the same administration route. (3) The compound of the present invention and the concomitant drug are separately produced to give two kinds of preparations which are administered by the same administration route only at the different times. (4) The compound of the present invention and the concomitant drug are separately produced to give two kinds of preparations which are administered simultaneously by different administration routes. (5) The compound of the present invention and the concomitant drug are separately produced to give two kinds of preparations which are administered by different administration routes at different times (for example, the compound of the present invention and the concomitant drug are administered in this order, or in, the reverse order).

### EXAMPLES

The present invention is explained in detail in the following by referring to Examples, Formulation Examples and Experimental Examples, which are not to be construed as limitative.

### Example A-1

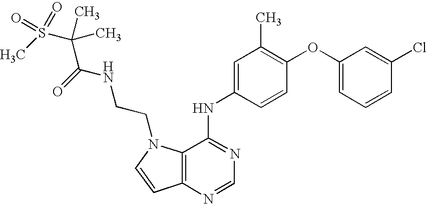 [2296] Production of N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide

To a solution of 5-(2-aminoethyl)-N-[3-chloro-4-(3-chlorophenoxy)phenyl]-5H-pyrrolo[3,2-d]pyrimidin-4-amine dihydrochloride (487 mg), 2-methyl-2-(methylsulfonyl)propanoic acid (249 mg) and 1-hydroxybenzotriazole (225 mg) in N,N-dimethylformamide (5.0 mL) were added triethylamine (0.69 mL) and 1-ethyl-3-(3-dimethylaminopropyl)carbodiimide hydrochloride (316 mg) under ice-cooling, and the mixture was stirred at room temperature for 15 hr. Water was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→90:10) and further recrystallized from ethyl acetate/diisopropyl ether to give the title lo compound (419 mg) as colorless crystals.

¹H-NMR (CDCl₃) δ: 1.70 (6H, s), 2.93 (3H, s), 3.60-3.80 (2H, m), 4.40-4.60 (2H, m), 6.46 (1H, d, J=2.8 Hz), 6.85-7.00 (2H, m), 7.00-1.15 (2H; m), 7.15-7.30 (2H, m), 7.30-7.40 (1H, m), 7.85-7.95 (1H, m), 8.00-8.05 (1H, m), 8.36 (1H, br s), 8.54 (1H, s).

### Example A-2

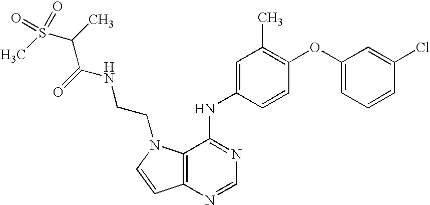 [2300] Production of N-[2-(4-{[3-chloro-4-(3-chlorophenoxy)phenyl]amino}-5H-pyrrolo[3,2-d]pyrimidin-5-yl)ethyl]-2-(methylsulfonyl)propanamide

To a solution of 5-(2-aminoethyl)-N-[3-chloro-4-(3-chlorophenoxy)phenyl]-5H-pyrrolo[3,2-d]pyrimidin-4-amine dihydrochloride (200 mg), 2-chloropropanoic acid (67 mg) and 1-hydroxybenzotriazole (90 mg) in N,N-dimethylformamide (4.0 mL) were added triethylamine (0.29 mL) and 1-ethyl-3-(3-dimethylaminopropyl)carbodiimide hydrochloride (126 mg) under ice-cooling, and the mixture was stirred at room temperature for 17 hr. Water was added to the reaction 30 mixture and the mixture was extracted with ethyl acetate. The organic layer was washed with brine and dried over anhydrous magnesium sulfate. After concentration under reduced pressure, the residue was dissolved in N,N-dimethylformamide (2 mL), sodium methanesulfinic acid (420 mg) and pyridine (0.40 mL) were added, and the mixture was stirred at 70° C. for 2 days. After cooling to room temperature, water was added to the reaction Mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→95:5) and further recrystallized from ethyl acetate/diisopropyl ether to give the title compound (97 mg) as colorless crystals.

¹H-NMR (CDCl₃) δ: 1.71 (3H, d, J=7.2 Hz), 2.98 (3H, s), 3.65-3.75 (2H, m), 3.81 (1H, q, J=7.2 Hz), 4.45-4.55 (2H, m), 6.61 (1H, d, J=3.3 Hz), 6.85-6.90 (1H, m), 6.90-6.95 (1H, m), 7.00-7.10 (2H, m), 7.20-7.30 (1H, m), 7.30-7.40 (1H, m), 7.75-7.85 (1H, m), 7.97 (1H, d, J=2.4 Hz), 8.28 (1H, s), 8.51 (1H, s).

### Example A-3

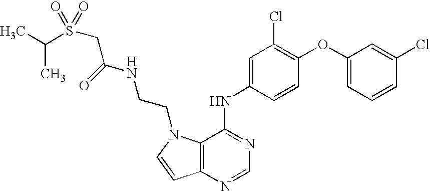

Production of N-[2-(4-{[3-chloro-4-(3-chlorophenoxy)phenyl]amino}-5H-pyrrolo[3,2-d]pyrimidin-5-yl)ethyl]-2-(isopropylsulfonyl)acetamide

### (i) Production of N-[2-(4-{[3-chloro-4-(3-chlorophenoxy)phenyl]amino}-5H-pyrrolo[3,2-d]pyrimidin-5-yl)ethyl]-2-(isopropylthio)acetamide

To a solution of 5-(2-aminoethyl)-N-[3-chloro-4-(3-chlorophenoxy)phenyl]-5H-pyrrolo[3,2-d]pyrimidin-4-amine dihydrochloride (300 mg), chloroacetic acid (87 mg) and 1-hydroxybenzotriazole (135 mg) in N,N-dimethylformamide (5.0 mL) were added triethylamine (0.43 mL) and 1-ethyl-3-(3-dimethylaminopropyl)carbodiimide hydrochloride (189 mg) under ice-cooling, and the mixture was stirred at room temperature for 18 hr. Water was added to the reaction mixture and the mixture was extracted with lo ethyl acetate. The organic layer was washed with brine and dried over anhydrous magnesium, sulfate. After concentration under reduced pressure, the residue was dissolved in N,N-dimethylformamide (2 mL)/tetrahydrofuran (4 mL), sodium propane-2-thiolate (605 mg) was added, and the mixture was stirred at room temperature for 6 hr. Aqueous sodium bicarbonate was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→95:5) to give the title compound (201 mg) as a white powder.

¹H-NMR (CDCl₃) δ: 1.24 (6H, d, J=6.9 Hz), 2.80-2.90 (1H, m), 3.33 (2H, s), 3.60-3.70 (2H, m), 4.45-4.55 (2H, m), 6.62 (1H, d, J=3.3 Hz), 6.85-6.90 (1H, m), 6.95-7.00 (1H, m), 7.00-7.05 (1H, m), 7.07 (1H, d, J=8.7 Hz), 7.20-7.30 (2H, m), 7.40-7.50 (1H, m), 7.73 (1H, dd, J=2.4, 8.7 Hz), 8.05 (1H, d, J=2.4 Hz), 8.51 (1H, s).

### (ii) Production of N-[2-(4-{[3-chloro-4-(3-chlorophenoxy)phenyl]amino}-5H-pyrrolo[3,2-d]pyrimidin-5-yl)ethyl]-2-(isopropylsulfonyl)acetamide

To a solution of N-[2-(4-{[3-chloro-4-(3-chlorophenoxy)phenyl]amino}-5H-pyrrolo[3,2-d]pyrimidin-5-yflethyl]-2-(isopropylthio)acetamide in methanol (6 mL)/water (1.5 mL) was added OXONE® monopersulfate compound (339 mg), and the mixture was stirred at room temperature for 21 hr. Water was added to the reaction mixture and the mixture was extracted with dichloromethane. The organic layer was dried over anhydrous magnesium sulfate and concentrated under reduced pressure. The residue was recrystallized from dichloromethane/methanol/diisopropyl ether to give the title compound (173 mg) as pale-yellow crystals.

¹H-NMR (DMSO-d₆) δ: 1.23 (6H, d, J=6.9 Hz), 3.40-3.65 (3H, m), 4.03 (2H, s), 4.50-4.70 (2H, m), 6.58-(1H, s), 6.90-6.95 (1H, m), 6.99 (1H, s), 7.15-7.25 (1H, m), 7.30 (1H, d, J=8.7 Hz), 7.40-7.50 (1H, m), 7.65-7.75 (1H, m), 7.79 (1H, s), 7.92 (1H, s), 8.53 (1H, s), 8.70-8.80 (1H, m), 9.28 (1H, br s).

### Example A-4

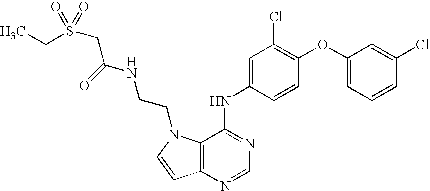 [2310] Production of N-[2-(4-{[3-chloro-4-(3-chlorophenoxy)phenyl]amino}-5H-pyrrolo[3,2-d]pyrimidin-5-yl)ethyl]-2-(ethylsulfonyl)acetamide

### (i) Production of N-[2-(4-{[3-chloro-4-(3-chlorophenoxy)phenyl]amino}-5H-pyrrolo[3,2-d]pyrimidin-5-yl)ethyl]-2-(ethylthio)acetamide

Using 5-(2-aminoethyl)-N-[3-chloro-4-(3-chlorophenoxy)phenyl]-5H-pyrrolo[3,2-d]pyrimidin-4-amine dihydrochloride (200 mg), ethylthioacetic acid (99 mg), 1-hydroxybenzotriazole (123 mg), triethylamine (0.57 mL), 1-ethyl-3-(3-dimethylaminopropyl)carbodiimide hydrochloride (173 mg) and N,N-dimethylformamide (4.0 mL) and in the same manner as in Example. A-1, the title compound (186 mg) was obtained as a white powder.

¹H-NMR (CDCl₃) δ: 1.24 (3H, t, J=7.5 Hz), 2.52 (2H, q, J=7.5 Hz), 3.32 (2H, s), 3.60-3.70 (2H, m), 4.45-4.55 (2H, m), 6.62 (1H, d, J=3.0 Hz), 6.88 (1H, d, J=8.1 Hz), 6.95-7.00 (1H, m), 7.00-7.10 (2H, m), 7.15-7.25 (1H, m), 7.40-7.50 (1H, m), 7.70-7.80 (1H, m), 8.05-8.10 (1H, m), 8.50 (1H, s), 8.51 (1H, s).
(ii) Production of N-[2-(4-{[3-chloro-4-(3-chlorophenoxy)phenyl]amino}-5H-pyrrolo[3,2-d]pyrimidin-5-yl)ethyl]-2-(ethylsulfonyl)acetamide

Using N-[2-(4-{[3-chloro-4-(3-chlorophenoxy)phenyl]amino}-5H-pyrrolo[3,2-d]pyrimidin-5-yl)ethyl]-2-(ethylthio)acetamide (180 mg), OXONE® monopersulfate compound (322 mg) and methanol (6 mL)/water (1.2 mL) and in the same manner as in Example A-3(ii), the title compound (149 mg) was obtained as colorless crystals.

¹H-NMR (DMSO-d₆) δ: 1.21 (3H, t, J=7.2 Hz), 3.22 (2H, q, J=7.2 Hz), 3.45-3.55 (2H, m), 4.03 (2H, s), 4.55-4.65 (2H, m), 6.55-6.60 (1H, m), 6.90-6.95 (1H, m), 6.99 (1H, s), 7.15-7.20 (1H, m), 7.29 (1H, d, J=8.7 Hz), 7.41 (1H, t, J=8.2 Hz), 7.65-7.75 (1H, m), 7.75-7.80 (1H, m), 7.93 (1H, s), 8.52 (1H, s), 8.72 (1H, br s), 9.22 (1H, br s).

### Example A-5

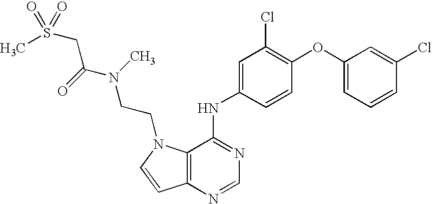 [2317] Production of N-[2-(4-{[3-chloro-4-(3-chlorophenoxy)phenyl]amino}-5H-pyrrolo[3,2-d]pyrimidin-5-yl)ethyl]-N-methyl-2-(methylsulfonyl)acetamide

### (i) Production of tert-butyl [2-{4-([3-chloro-4-(3-chlorophenoxy)phenyl]amino}-5H-pyrrolo[3,2-d]pyrimidin-5-yl)ethyl]methylcarbamate

A mixture of tert-butyl [2-(4-chloro-5H-pyrrolo[3,2-d]pyrimidin-5-yl)ethyl]methylcarbamate (2.56 g), 3-chloro-4-(3-chlorophenoxy)aniline (2.51 g) and isopropyl alcohol (25 mL) was stirred at. 80° C. for 18 hr. After cooling to room temperature, the mixture was stirred for 5 hr. The precipitate was collected by filtration, and washed with diisopropyl ether to give the title compound (3.72 g) as a white powder.

¹H-NMR (CDCl₃) δ: 1.52 (9H, s), 3.01 (3H, s), 3.50-3.60 (2H, m), 4.40-4.50 (2H, m), 6.60 (1H, d, J=3.0 Hz), 6.85-6.95 (1H, m), 6.95-7.00 (1H, m), 7.00-7.05 (1H, m), 7.07 (1H, d, J=9.0 Hz), 7.15-7.25 (2H, m), 7.90 (1H, d, J=9.0 Hz), 8.01 (1H, br s), 8.52 (1H, s), 8.83 (1H, s).

### (ii) Production of N-[3-chloro-4-(3-chlorophenoxy)phenyl]-5-[2-(methylamino)ethyl]-5H-pyrrolo[3,2-d]pyrimidin-4-amine dihydrochloride

A mixture of tert-butyl [2-(4-{[3-chloro-4-(3-chlorophenoxy)phenyl]amino}-5H-pyrrolo[3,2-d]pyrimidin-5-yl)ethyl]methylcarbamate (3.72 g) and 10% (W/W) hydrochloric acid/methanol (30 mL) was stirred at 65° C. for 24 hr. The reaction mixture was concentrated under reduced pressure, and the precipitate was collected by filtration, and washed with diethyl ether to give the title compound (2.70 g) as pale-yellow crystals.

¹H-NMR (DMSO-d₆) δ: 2.50-2.60 (3H, m), 3.30-3.50 (2H, m), 5.00-5.20 (2H, m), 6.75 (1H, d, J=3.0 Hz), 6.90-7.00 (1H, m), 7.02 (1H, s), 7.21 (1H, d, J=7.8 Hz), 7.32 (1H, d, J=8.7 Hz), 7.44 (1H, t, J=8.1 Hz), 7.66 (1H, d, J=8.7 Hz), 7.93 (1H, s), 8.07 (1H, d, J=3.0 Hz), 8.73 (1H, s), 9.10-9.30 (2H, m), 10.17 (1H, br s).

### (iii) Production of N-[2-(4-{[3-chloro-4-(3-chlorophenoxy)phenyl]amino}-5H-pyrrolo[3,2-d]pyrimidin-5-yl)ethyl]-N-methyl-2-(methylsulfonyl)acetamide

Using N-[3-chloro-4-(3-chlorophenoxy)phenyl]-5-[2-(methylamino)ethyl]-5H-pyrrolo[3,2-d]pyrimidin-4-amine dihydrochloride (200 mg), methylsulfonylacetic acid (83 mg), 1-hydroxybenzotriazole (87 mg), triethylamine (0.28 mL), 1-ethyl-3-(3-dimethylaminopropyl)carbodiimide io hydrochloride (123 mg) and N,N-dimethylformamide (5.0 mL) and in the same manner as in Example A-1, the title. compound (164 mg) was obtained as colorless crystals.

¹H-NMR (CDCl₃) δ: 3.17 (3H, s), 3.33 (3H, s), 3.70-3.85 (2H, m), 4.17 (2H, s), 4.45-4.55 (2H, m), 6.63 (1H, d, J=3.0 Hz), 6.85-6.95 (2H, m), 7.00-7.10 (2H, m), 7.20-7.30 (2H, m), 7.82 (1H, dd, J=2.7 Hz, 9.0 Hz), 7.92 (1H, d, J=2.7 Hz), 8.44 (1H, s), 8.52 (1H, s).

### Example A-6

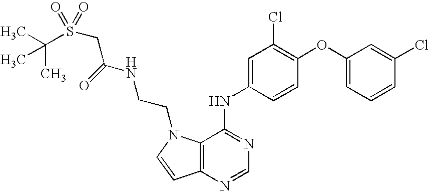 [2325] Production of 2-(tert-butylsulfonyl)-N-[2-(4-{[3-chloro-4-(3-chlorophenoxy)phenyl]amino}-5H-pyrrolo[3,2-d]pyrimidin-5-yl)ethyl]acetamide

### (i) Production of 2-(tert-butylthio)-N-[2-(4-{[3-chloro-4-(3-chlorophenoxy)phenyl]amino}-5H-pyrrolo[3,2-d]pyrimidin-5-yl)ethyl]acetamide

To a solution of 5-(2-aminoethyl)-N-[3-chloro-4-(3-chlorophenoxy)phenyl]-5H-pyrrolo[3,2-d]pyrimidin-4-amine dihydrochloride (200 mg), chloroacetic acid (58 mg) and 1-hydroxybenzotriazole (90 mg) in N,N-dimethylformamide (4.0 mL) were added triethylamine (0.29 mL) and 1-ethyl-3-(3-dimethylaminopropyl)carbodiimide hydrochloride (126 mg) under ice-cooling, and the mixture was stirred at room temperature for 4 hr. Water was added to the reaction mixture and the mixture was extracted with ethyl acetate. The organic layer was washed with brine and dried over anhydrous magnesium sulfate. After concentration under reduced pressure, the residue was dissolved in N,N-dimethylformamide (2 mL)/tetrahydrofuran (4 mL), sodium 2-methylpropane-2-thiolate (511 mg) was added, and the mixture was stirred at room temperature for 2 hr. Aqueous sodium bicarbonate was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→95:5) to give the title compound (159 mg) as a white powder.

¹H-NMR (CDCl₃) δ: 1.30 (9H, s), 3.33 (2H, s), 3.60-3.70 (2H, m), 4.40-4.50 (2H, m), 6.61 (1H, d, J=3.3 Hz), 6.85-6.90 (1H, m), 6.95-7.00 (1H, m), 7.00-7.05 (1H, m), 7.07 (1H, d, J=9.0 Hz), 7.15-7.25 (2H, m), 7.45-7.55 (1H, m), 7.73 (1H, dd, J=3.0 Hz, 9.0 Hz), 8.06 (1H, d, J=2.7 Hz), 8.51 (1H, s), 8.56 (1H, s).

### (ii) Production of 2-(tert-butylsulfonyl)-N-[2-(4-{[3-chloro-4-(3-chlorophenoxy)phenyl]amino}-5H-pyrrolo[3,2-d]pyrimidin-5-yl)ethyl]acetamide

Using 2-(tert-butylthio)-N-[2-(4-{[3-chloro-4-(3-chlorophenoxy)phenyl]amino}-5H-pyrrolo[3,2-d]pyrimidin-5-yl)ethyl]acetamide (159 mg), OXONE® monopersulfate compound (269 mg) and methanol (5 mL)/water (1.5 mL) and in the same manner as in Example A-3(ii), the title compound (99 mg) was obtained as pale-yellow crystals.

¹H-NMR (95% CDCl₃+5% DMSO-d₆) δ: 1.43 (9H, s), 3.50-3.70 (2H, m), 4.00 (2H, s), 4.60-4.70 (2H, m), 6.60 (1H, d, J=3.0 Hz), 6.85-6.95 (2H, m), 7.05-7.15 (2H, m), 7.31 (1H, t, J=8.1 Hz), 7.60-7.70 (2H, m), 7.92 (1H, s), 8.49 (1H, s), 8.80-8.90 (1H, m), 9.30-9.50 (1H, m).

### Example A-7

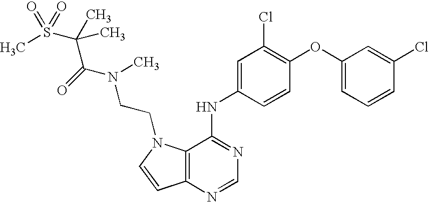 [2331] Production of N-[2-(4-{[3-chloro-4-(3-chlorophenoxy)phenyl]amino}-5H-pyrrolo[3,2-d]pyrimidin-5-yl)ethyl]-N,2-dimethyl-2-(methylsulfonyl)propanamide

To a solution of N-[3-chlorO-4-(3-chlorophenoxy)phenyl]-5-[2-(methylamino)ethyl]-5H-pyrrolo[3,2-d]pyrimidin-4-amine dihydrochloride (200 mg) and 2-methyl-2-(methylsulfonyl)propanoic acid (100 mg) in N,N-dimethylformamide (5.0 mL) were added triethylamine (0.28 mL) and diethyl cyanophosphonate (0.097 mL) under ice-cooling, and the mixture was stirred at room temperature for 25 hr. Aqueous sodium bicarbonate was added to the reaction mixture and the 20 mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→90:10) and further recrystallized from ethyl acetate/diisopropyl ether to give the title compound (94 mg) as pale-yellow crystals.

¹H-NMR (CDCl₃) δ: 1.85 (6H, s), 2.97 (3H, s), 3.47 (3H, s), 3.70-3.80 (2H, m), 4.40-4.50 (2H, m), 6.63 (1H, d, J=3.6 Hz), 6.85-6.95 (2H, m), 7.00-7.05 (1H, m), 7.06 (1H, d, J=8.7 Hz), 7.20-7.30 (2H, m), 7.90-8.00 (1H, m), 8.01 (1H, d, J=2.4 Hz), 8.52 (1H, s), 8.69 (1H, br s)

### Example A-8

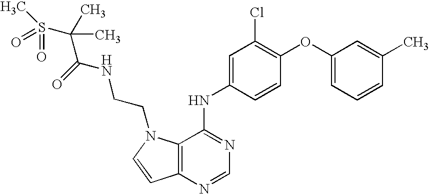 [2335] Production of N-[2-(4-{[3-chloro-4-(3-methylphenoxy)phenyl]amino}-5H-pyrrolo[3,2-d]pyrimidin-5-yl)ethyl]-2-methyl-2-(methylsulfonyl)propanamide

### (i) Production of 5-(2-aminoethyl)-N-[3-chloro-4-(3-methylphenoxy)phenyl]-5H-pyrrolo[3,2-d]pyrimidin-4-amine dihydrochloride

A mixture of tert-butyl [2-(4-chloro-5H-pyrrolo[3,2-d]pyrimidin-5-yl)ethyl]carbamate (594 mg), 3-chloro-4-(3-methylphenoxy)aniline (467 mg) and isopropyl alcohol (10 mL) was stirred at 80° C. for 6 hr. To the reaction mixture was added aqueous sodium hydrogencarbonate solution, and, the mixture was extracted with ethyl acetate. The organic layer was washed with saturated brine and dried over anhydrous magnesium sulfate. The solvent was evaporated under reduced pressure, and the obtained residue was purified by silica gel column chromatography (eluent, ethyl acetate:hexane=50:50→100:0). The objective fractions were concentrated under reduced pressure. To a solution of the residue in methanol (10 mL) was added concentrated hydrochloric acid (3 mL), and the mixture was stirred at room temperature overnight and further at 60° C. for 3 hr. The reaction mixture was concentrated under reduced pressure. Isopropyl alcohol and toluene were added to the residue, and the mixture was concentrated under reduced pressure. Methanol was added to the residue, and the mixture was concentrated under reduced pressure. Isopropyl alcohol and diisopropyl ether were added to the residue, and the precipitated solid was collected by filtration to give the title compound (805 mg) as a pale-yellow powder.

¹H-NMR (DMSO-d6) δ: 2.31(3H, s), 3.23-3.37 (2H, m), 5.04 (2H, t, J=6.2 Hz), 6.72-6.80 (2H, m), 6.83 (1H, m), 6.98 (1H, d, J=7.5 Hz), 7.18 (1H,^{,}d, J=8.9 Hz), 7.29 (1H, t, J=7.8 Hz), 7.59 (1H, dd, J=8.8, 2.5 Hz), 7.87 (1H, d, J=2.5 Hz), 8.07 (1H, d, J=3.2 Hz), 8.35 (3H, br s), 8.73 (1H, s), 10.15 (1H, br s).

### (ii) Production of N-[2-(4-{[3-chloro-4-(3-methylphenoxy)phenyl]amino}-5H-pyrrolo[3,2-d]pyrimidin-5-yl)ethyl]-2-methyl-2-(methylsulfonyl)propanamide

A mixture of 5-(2-aminoethyl)-N-[3-chloro-4-(3-methylphenoxy)phenyl]-5H-pyrrolo[3,2-d]pyrimidin-4-amine dihydrochloride (140 mg), 2-methyl-2-(methylsulfonyl)propanoic acid (75 mg), 1-ethyl-3-(3-dimethylaminopropyl)carbodiimide hydrochloride (86 mg), 1-hydroxybenzotriazole (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to basic silica gel column chromatography (eluent, methanol:ethyl acetate=0:100→20:80). The objective fractions were concentrated under reduced pressure. The residue was crystallized from ethyl acetate-diisopropyl ether to give the title compound (155 mg) as a white powder.

¹H-NMR (CDCl₃) δ: 1.69 (6H, s), 2.33 (3H, s), 2.93 (3H, s), 3.61-3.74 (2H, m), 4.41-4.51 (2H, m), 6.61 (1H, d, J=3.3 Hz), 6.75-6.84 (2H, m), 6.89 (1H, d, J=7.7 Hz), 7.02 (1H, d, J=8.8 Hz), 7.16-7.24 (2H, m), 7.34 (1H, t, J=5.8 Hz), 7.80 (1H, dd, J=8.8 Hz, 2.5 Hz), 7.97 (1H, d, J=2.5 Hz), 8.31 (1H, br s), 8.51 (1H, s).

### Example A-9

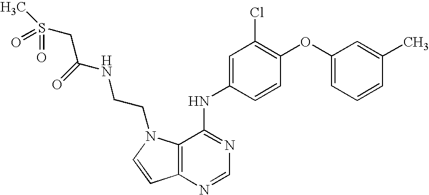 [2341] Production of N-[2-(4-{[3-chloro-4-(3-methylphenoxy)phenyl]amino}-5H-pyrrolo[3,2-d]pyrimidin-5-yl)ethyl]-2-(methylsulfonyl)acetamide

A mixture of 5-(2-aminoethyl)-N-[3-chloro-4-(3-methylphenoxy)phenyl]-5H-pyrrolo[3,2-d]pyrimidin-4-amine dihydrochloride (140 mg), methylsulfonylacetic acid (62 mg), 1-ethyl-3-(3-dimethylaminopropyl)carbodiimide hydrochloride (86 mg), 1-hydroxybenzotriazole (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine and dried over anhydrous magnesium sulfate. The solvent was evaporated under reduced pressure and the obtained residue was subjected to basic silica gel column chromatography (eluent, methanol:ethyl acetate=0:100→15:85). The objective fractions were concentrated under reduced pressure. The residue was crystallized from ethyl acetate-diisopropyl ether to give the title compound (147 mg) as a white powder.

¹H-NMR (CDCl₃) δ: 2.33 (3H, s), 3.13 (3H,$), 3.63-3.76 (2H, m), 3.70 (2H, s), 4.41-4.53 (2H, m), 6.58 (1H, d, J=3.3 Hz), 6.75-6.84 (2H, m), 6.90 (1H, d, J=7.4 Hz), 7.01 (1H, d, J=8.7 Hz), 7.16-7.24 (2H, m), 7.55-7.64 (1H, m), 7.69 (1H, dd, J=8.7, 2.7 Hz), 7.89 (1H, d, J=2.7 Hz), 8.14 (1H, br s), 8.48 (1H, s).

### Example A-10

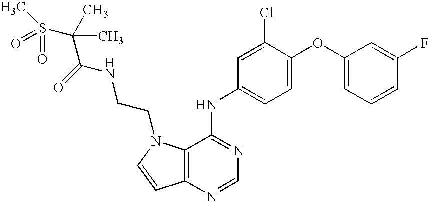 [2345] Production of N-[2-(4-{[3-chloro-4-(3-fluorophenoxy)phenyl]amino}-5H-pyrrolo[3,2-d]pyrimidin-5-yl)ethyl]-2-methyl-2-(methylsulfonyl)propanamide

### (i) Production of 5-(2-aminoethyl)-N-[3-chloro-4-(3-fluorophenoxy)phenyl]-5H-pyrrolo[3,2-d]pyrimidin-4-amine dihydrochloride

A mixture of tert-butyl [2-(4-chloro-5H-pyrrolo[3,2-d]pyrimidin-5-yl)ethyl]carbamate (594 mg), 3-chloro-4-(3-fluorophenoxy)aniline (475 mg) and isopropyl alcohol (10 mL) was stirred at 80° C. for 6 hr. An aqueous sodium hydrogencarbonate solution was added to the reaction mixture, and the mixture was extracted with ethyl acetate. The ethyl acetate layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure. Methanol (10 mL), tetrahydrofuran (1 mL) and concentrated hydrochloric acid (3 mL) were added to the residue, and the mixture was stirred at room temperature overnight and further stirred at 60° C. for 3 hr. The reaction mixture was concentrated under reduced pressure. Isopropyl alcohol and toluene were added tb the residue, and the mixture was concentrated under reduced pressure. Methanol was added to the residue, and the mixture was concentrated under reduced pressure. Isopropyl alcohol and diisopropyl ether were added to the residue and the precipitated solid was collected by filtration, to give the title compound (809 mg) as a pale-yellow powder.

¹H-NMR (DMSO-d₆) δ: 3.22-3.39 (2H, m), 5.09 (2H, t, J=6.3 Hz), 6.73-6.82 (2H, m), 6.83-6.92 (1H, m), 6.96-7.05 (1H, m), 7.31 (1H, d, J=8.9 Hz), 7.39-7.51 (1H, m), 7.66 (1H, dd, J=2.4 Hz, 8.9 Hz), 7.93 (1H, d, J=2.4 Hz), 8.10 (1H, d, J=3.2 Hz), 8.42 (3H, br s), 8.74 (1H, s), 10.30 (1H, br s).

### (ii) Production of N-[2-(4-{[3-chloro-4-(3-fluorophenoxy)phenyl]amino}-5H-pyrrolo[3,2-d]pyrimidin-5-yl)ethyl]-2-methyl-2-(methylsulfonyl)propanamide

A mixture of 5-(2-aminoethyl)-N-[3-chloro-4-(3-fluorophenoxy)phenyl]-5H-pyrrolo[3,2-d]pyrimidin-4-amine dihydrochloride (141 mg), 2-methyl-2-(methylsulfonyl)propanoic acid (75 mg), 1-ethyl-3-(3-dimethylaminopropyl)carbodiimide hydrochloride (86 mg), 1-hydroxybenzotriazole (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to basic silica gel column chromatography (eluent, methanol:ethyl acetate=0:100→20:80). The objective fractions were concentrated under reduced pressure. The residue was crystallized from ethyl acetate-diisopropyl ether to give the title compound (161 mg) as a white powder.

¹H-NMR (CDCl₃) δ: 1.70 (6H, s), 2.93 (3H, s), 3.63-3.74 (2H, m), 4.42-4.53 (2H, m), 6.63 (1H, d, J=3.3 Hz), 6.64-6.71 (1H, m), 6.74-6.82 (2H, m), 7.09 (1H, d, J=8.9 Hz), 7.19-7.12 (2H, m), 7.37 (1H, t, J=5.8 Hz), 7:88 (1H, dd, J=2.7 Hz, 8.9 Hz), 8.02 (1H, d, J=2.7 Hz), 8.36 (1H, br s), 8.53 (1H, s).

### Example A-11

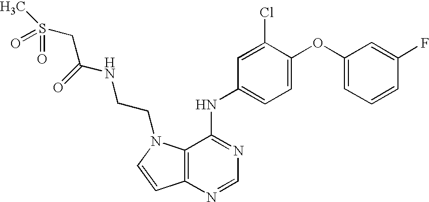 [2351] Production of N-[2-(4-{[3-chloro-4-(3-fluorophenoxy)phenyl]amino}-5H-pyrrolo[3,2-d]pyrimidin-5-yl)ethyl]-2-(methylsulfonyl)acetamide

A mixture of 5-(2-aminoethyl)-N-[3-chloro-4-(3-fluorophenoxy)phenyl]-5H-pyrrolo[3,2-d]pyrimidin-4-amine dihydrochloride (141 mg), methylsulfonylacetic acid (62 mg), 1-ethyl-3-(3-dimethylaminopropyl)carbodiimide hydrochloride (86 mg), 1-hydroxybenzotriazole (69 mg), triethylamine (0.100 mL) and N,N-dimethylformamide (3 mL) was stirred at room temperature overnight. Water was added to the reaction mixture, and the mixture was extracted with ethyl acetate. The ethyl acetate layer was washed successively with water and saturated brine, and dried over anhydrous magnesium sulfate. The solvent was evaporated under reduced pressure and the obtained residue was subjected to basic silica gel column chromatography (eluent, methanol:ethyl acetate=0:100→15:85). The objective fractions were concentrated under reduced pressure. The residue was crystallized from ethyl acetate-diisopropyl ether to give the title compound (146 mg) as a white powder.

¹H-NMR (CDCl₃) δ: 3.14 (3H, s), 3.64-3.76 (2H, m), 3.98 (2H, s), 4.43-4.54 (2H, m), 6.59 (1H, d, J=3.3 Hz), 6.63-6.70 (1H, m), 6.73-6.82 (2H, m), 7.08 (1H, d, J=8.9 Hz), 7.18-7.31 (2H, m), 7.57-7.65 (1H, m), 7.75 (1H, dd, J=2.5 Hz, 8.9 Hz), 7.93 (1H, d, J=2.5 Hz), 8.19 (1H, br s), 8.49 (1H, s).

### Example A-12

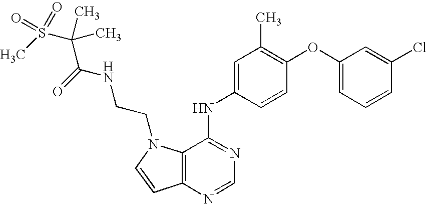 [2355] Production of N-[2-(4-{[4-(3-chlorophenoxy)-3-methylphenyl]amino}-5H-pyrrolo[3,2-d]pyrimidin-5-yl)ethyl]-2-methyl-2-(methylsulfonyl)propanamide

### (i) Production of tert-butyl [2-(4-{[4-(3-chlorophenoxy)-3-methylphenyl]amino}-5H-pyrrolo[3,2-d]pyrimidin-5-yl)ethyl]carbamate

A solution of tert-butyl [2-(4-chloro-5H-pyrrolo[3,2-d]pyrimidin-5-yl)ethyl]carbamate (1.0 g) and 3-methyl-4-[3-chlorophenoxy]aniline (1.18 g) in isopropyl alcohol (10 mL) was stirred at 80° C. for 12 hr. Aqueous sodium bicarbonate was added to the reaction mixture and the mixture was extracted with ethyl acetate. The organic layer was washed with saturated brine and dried over anhydrous magnesium sulfate. After concentration under reduced pressure, the residue was separated and purified by silica gel column chromatography (eluent, hexane:ethyl acetate=8:2→ethyl acetate) to give the title compound (1.7 g) as colorless crystals.

¹H-NMR (CDCl₃) δ: 1.47 (9H, s), 2.20 (3H, s), 3.48 (2H, m), 4.45 (2H,m), 5.16 (1H, m), 6.57 (1H, d, J=3 Hz), 6.80-7.00 (4H, m), 7.10-7.30 (2H, m), 7.68 (2H, m), 8.40 (1H, br s), 8.49 (1H, s).

### (ii) Production of 5-(2-aminoethyl)-N-[4-(3-chlorophenoxy)-3methylphenyl]-5H-pyrrolo[3,2-d]pyrimidin-4-amine dihydrochloride

A mixture of tert-butyl [2-(4-{[4-(3-chlorophenoxy)-3-methylphenyl]amino}-5H-pyrrolo[3,2-d]pyrimidin-5-yl)ethyl]carbamate (1.6 g), 2N hydrochloric acid (23 mL) and tetrahydrofuran (46 mL) was stirred at 60° C. for 20 hr. The solvent was evaporated under reduced pressure, ethanol was added, and the mixture was further concentrated. The resulting crystals were collected by filtration. The crystals were washed with isopropyl ether to give the title compound (1.35 g) as a pale-yellow powder.

¹H-NMR (DMSO-d₆) δ: 2.19 (3H, s), 3.30 (2H, m), 5.04 (2H, m), 6.72 (1H, d, J=3 Hz), 6.80-7.00 (2H, m), 7.08 (1H, d, J=9 Hz), 7.16 (1H, dd, J=2 Hz, 8 Hz), 7.30-7.50 (2H, m), 7.54 (1H, m), 8.06 (1H, m), 8.40 (3H, br s), 8.68 (1H, s), 10.00 (1H, br s).

### (iii) Production of N-[2-(4-{[4-(3-chlorophenoxy)-3-methylphenyl]amino}-5H-pyrrolo[3,2-d]pyrimidin-5-yl)ethyl]-2-methyl-2-(methylsulfonyl)propanamide

A mixture of 5-(2-aminoethyl)-N-[4-(3-chlorophenoxy)-3-methylphenyl]-5H-pyrrolo[3,2-d]pyrimidin-4-amine dihydrochloride (167 mg), 2-methyl-2-(methylsulfonyl)propanoic acid (89 mg), 1-ethyl-3-(3-dimethylaminopropyl)carbodiimide hydrochloride (103 mg), 1-hydroxybenzotriazole (72.5 mg), triethylamine (0.15 mL) and N,N-dimethylformamide (6.9 mL) was stirred at room temperature for 16 hr. Water was added to the reaction mixture and the mixture was extracted with ethyl acetate. The organic layer was washed successively with water and saturated brine, and dried over anhydrous magnesium sulfate. After concentration under reduced pressure, the residue was separated and purified by basic silica gel column chromatography (eluent, ethyl acetate→ethyl acetate:methanol=85:15) to give the title compound (179 mg) as colorless crystals.

¹H-NMR (DMSO-d₆) δ: 1.42 (6H, s), 2.14 (3H, s), 2.96 (3H, s), 3.47 (2H, q, J=6 Hz), 4.56 (2H, t, J=6 Hz), 6.45 (1H, d, J=3 Hz), 6.80-6.90 (2H, M), 7.02 (1H, d, J=9 Hz), 7.11 (1H, dd, J=1 Hz, 8 Hz), 7.37 (1H, t, J=8 Hz), 7.52 (1H, d, J=3 Hz), 7.58 (2H, m), 8.20 (1H, t, J=6 Hz), 8.28 (1H, s), 8.49 (1H, br s).

### Example A-13

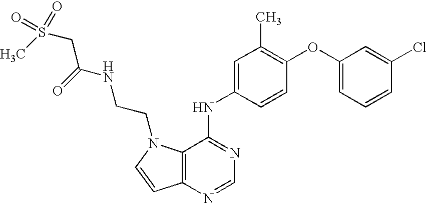 [2363] Production of N-[2-(4-{[4-(3-chlorophenoxy)-3-methylphenyl]amino}-5H-pyrrolo[3,2-d]pyrimidin-5-yl)ethyl]-2-(methylsulfonyl)acetamide

A mixture of 5-(2-aminoethyl)-N-[4-(3-chlorophenoxy)-3-methylphenyl]-5H-pyrrolo[3,2-d]pyrimidin-4-amine dihydrochloride (167 mg), methylsulfonylacetic acid (74 mg), 1-ethyl-3-(3-dimethylaminopropyl)carbodiimide hydrochloride (103 mg), 1-hydroxybenzotriazole (72.5 mg), triethylamine (0.15 mL) and N,N-dimethylformamide (6.9 mL) was stirred at room temperature for 16 hr. Water was added to the reaction mixture and the mixture was extracted with ethyl acetate. The organic layer was washed successively with water and saturated brine, and dried over anhydrous magnesium sulfate. After concentration under reduced pressure, the residue was separated and purified by basic silica gel column chromatography (eluent, ethyl acetate→ethyl acetate:methanol=85:15) to give the title compound (177 mg) as colorless crystals.

¹H-NMR (DMSO-d₆) δ: 2.13 (3H, s), 3.09 (3H, s), 3.45 (2H, q, J=6 Hz), 4.05 (2H, s), 4.55 (2H, t, J=6 Hz), 6.46 (1H, d, J=3 Hz), 6.80-6.95 (2H, m), 7.00 (1H, d, J=9 Hz), 7.11 (1H, m), 7.37 (1H, t, J=8 Hz), 7.56 (3H, m), 8.28 (1H, s), 8.52 (1H, br s), 8.66 (1H, m).

### Example A-14

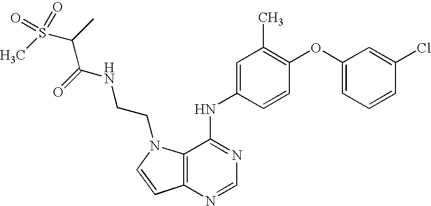 [2367] Production of N-[2-(4-{[4-(3-chlorophenoxy)-3-methylphenyl]amino}-5H-pyrrolo[3,2-d]pyrimidin-5-yl)ethyl]-2-(methylsulfonyl)propanamide

A mixture of 5-(2-aminoethyl)-N-[4-(3-chlorophenoxy)-3-methylphenyl]-5H-pyrrolo[3,2-d]pyrimidin-4-amine dihydrochloride (192 mg), 2-chloropropanoic acid (0.057 mL), 1-ethyl-3-(3-dimethylaminopropyl)carbodiimide hydrochloride (126 mg), 1-hydroxybenzotriazole (90 mg), triethylamine (0.29 mL) and N,N-dimethylformamide (4 mL) was stirred at room temperature for 16 hr. Water was added to the reaction mixture and the mixture was extracted with ethyl acetate. The organic layer was washed successively with water and saturated brine, and dried over anhydrous magnesium sulfate. After concentration under reduced pressure, the residue was separated and purified by silica gel column chromatography (eluent, ethyl acetate→ethyl acetate:methanol=90:10), and the fraction containing 2-chloro-N-[2-(4-{[4-(3-chlorophenoxy)-3-methylphenyl]amino}-5H-pyrrolo[3,2-d]pyrimidin-5-yl)ethyl]propanamide was concentrated under reduced pressure. The residue was dissolved in N,N-dimethylformamide (4 mL) and pyridine (0.4 mL), sodium methanesulfinic acid (420 mg) was added and the mixture was stirred at 70° C. for 2 days. After cooling to room temperature, water was added to the reaction mixture and the mixture was extracted with ethyl acetate. The organic layer was washed successively with water and saturated brine, and dried over anhydrous magnesium sulfate. After concentration under reduced pressure, the residue was separated and purified by basic silica gel column chromatography (eluent, ethyl acetate→ethyl acetate:methanol=85:15) to give the title compound (116 mg) as colorless crystals.

¹H-NMR (DMSO-d₆) δ: 1.36 (3H, d, J=7 Hz), 2.13 (3H, s), 2.95 (3H, s), 3.50 (2H, m), 3.82 (1H, m), 4.53 (2H, m), 6.46 (1H, d, J=3 Hz), 6.80-6.90 (2H, m), 7.01 (1H, d, J=9 Hz), 7.10 (1H, d, J=8 Hz), 7.37 (1H, t, J=8 Hz), 7.57 (3H, m), 8.28 (1H, s), 8.49 (1H, br s), 8.59 (1H, t, J=6 Hz).

### Example A-15

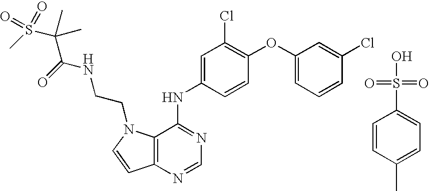 [2371] Production of N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide p-toluenesulfonate

Ethyl acetate (200 mL) and ethanol (70 mL) were added to N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide (9.0 g), the mixture was dissolved by heating at 65° C., and p-toluenesulfonic acid monohydrate (3.04 g) was added. The mixture was stood at room temperature under light shielding for 23 hr and the resulting crystals were collected by filtration. The crystals were washed with a small amount of ethyl acetate and diisopropyl ether to give the title compound (11.5 g) as colorless crystals.

¹H-NMR (DMSO-d₆) δ: 1.40 (6H, s), 2.28 (3H, s), 2.93 (3H, s), 3.50-3.60 (2H, m), 4.65-4.75 (2H, m), 6.65 (1H, d, J=3.0 Hz), 6.90-7.00 (1H, m), 7.00-7.05.(1H, m), 7.10 (2H, d, J=7.8 Hz), 7.20-7.25 (1H, m), 7.35 (1H, d, J=9.0 Hz), 7.40-7.50 (3H, m), 7.60-7.70 (1H, m), 7.89 (1H, d, J=3.0 Hz), 7.91 (1H, d, J=1.8 Hz), 8.15-8.25 (1H, m), 8.74 (1H, s), 9.80 (1H, br s). elemental analysis for C₃₂H₃₃Cl₂N₅O₇S₂

Calculated: C,52.32; H,4.53; N,9.53.

Found : C,52.35; H,4.54; N,9.49.

mp 217-218° C.

### Example A-16

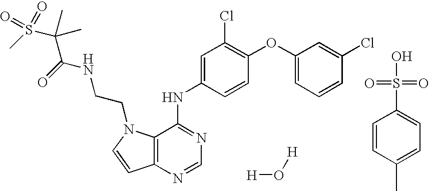 [2378] Production of N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide p-toluenesulfonate monohydrate

Acetone (20 mL) was added to N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide (500 mg), and the mixture was dissolved by heating at 40° C., and p-toluenesulfonic acid monohydrate (168 mg) was added. The mixture was stood at room temperature under light shielding for 4 days, and concentrated under reduced pressure. Ethyl acetate (12 mL) and ethanol (4 mL) were added to the residue, and the mixture was dissolved by heating at 60° C. The mixture was stood at room temperarure for 17 hr under light shielding, and resulting crystals were collected by filtration. The crystals were washed with diisopropyl ether to give the title compound (543 mg) as colorless crystals.

¹H-NMR (DMSO-d₆) δ: 1.40 (6H, s), 2.29 (3H, s), 2.93 (3H, s), 3.50-3.60 (2H, m), 4.65-4.75 (2H, m), 6.65 (1H, d, J=3.0 Hz), 6.90-7.00 (1H, m), 7.00-7.05.(1H, m), 7.10 (2H, d, J=7.8 Hz), 7.20-7.25 (1H, m), 7.35 (1H, d, J=9.0 Hz), 7.40-7.50 (3H, m), 7.67 (1H, dd, J=2.4 Hz, 9.0 Hz), 7.88 (1H, d, J=3.0 Hz), 7.92 (1H, d, J=2.4 Hz), 8.15-8.25 (1H, m), 8.73 (1H, s), 9.76 (1H, br s).

elemental analysis for C₃₂H₃₃Cl₂N₅O₇S₂.1.0H₂O

Calculated: C,51.06; H,4.69; N,9.30.

Found: C,50.49; H,4.52; N,9.23.

mp 216-217° C.

### Example A-17

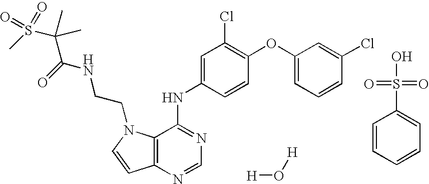 [2386] Production of N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide benzenesulfonate monohydrate

To N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide (400 mg) were added ethyl acetate (12 mL) and ethanol (4 mL), and the mixture was dissolved by heating at 60° C., and benzenesulfonic acid monohydrate (132 mg) was added. The mixture was stood at room temperature for 17 hr under light shielding and concentrated under reduced pressure, and ethyl acetate (10 mL) was added to the residue. The to mixture was stood at room temperature for 17 hr under light shielding, and resulting crystals were collected by filtration. The crystals were washed with diisopropyl ether to give the title compound (447 mg) as colorless crystals.

¹H-NMR (DMSO-d₆) δ: 1.41 (6H, s), 2.93 (3H, s), 3.50-3.60 (2H, m), 4.65-4.75 (2H, m), 6.65 (1H, d, J=3.0 Hz), 6.95-7.00 (1H, m), 7.00-7.05 (1H; m), 7.20-7.25 (1H, m), 7.25-7.35 (3H, m), 7.35 (1H, d, J=8.4 Hz), 7.45 (1H, t, J=8.4 Hz), 7.55-7.65 (2H, m), 7.67 (1H, dd, J=2.4, 8.7 Hz), 7.88 (1H, d, J=3.0 Hz), 7.93 (1H, d, J=2.4 Hz), 8.20-8.25 (1H, m), 8.73 (1H, s), 9.74 (1H, br s).

elemental analysis for C₃₁H₃₁Cl₂N₅O₇S₂.1.0H₂O

Calculated: C,50.41; H,4.50; N,9.48.

Found : C,50.53; H,4.43; N,9.48.

mp 142-144° C.

### Example A-18

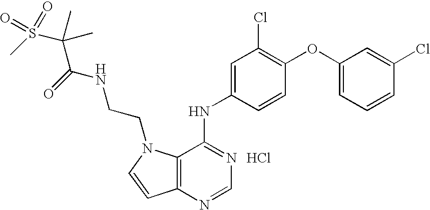

### Production of N-[2-(4-{[3-chloro-4-(3-chlorophenoxy)phenyl]amino}-5H-pyrrolo[3,2-d]pyrimidin-5-yl)ethyl]-2-methyl⁻2-(methylsulfonyl)propanamide hydrochloride

Acetone (20 mL) was added to N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide (400 mg), and the mixture was dissolved by heating at 40° C. 4N Hydrogen chloride/ethyl acetate solution (0.196 mL) was added. The mixture was stood at room temperature for 4 days under light shielding, and resulting crystals were collected by filtration. The crystals were washed with diisopropyl ether to give the title compound (401 mg) as pale-yellow crystals.

¹H-NMR (DMSO-d₆) δ: 1.40 (6H, s), 2.93 (3H, s), 3.50-3.65 (2H, m), 4.4.70-4.80 (2H, m), 6.65 (1H, d, J=3.0 Hz), 6.90-7.00 (1H, m), 7.00-7.05 (1H, m), 7.20-7.25 (1H, m), 7.35 (1H, d, J=8.7 Hz), 7.45 (1H, t, J=8.1 Hz), 7.68 (1H, dd, J=2.4 Hz, 8.7 Hz), 7.89 (1H, d, J=3.0 Hz), 7.94 (1H, d, J=2.4 Hz), 8.20-8.30 (1H, m), 8.73 (1H, s), 9.89 (1H, br s).

elemental analysis for C₂₅H₂₆Cl₃N₅O₄S

Calculated: C,50.13; H,4.38; N,11.69.

Found: C,49.70; H,4.41; N,11.48.

mp 194-195° C.

### Example A-19

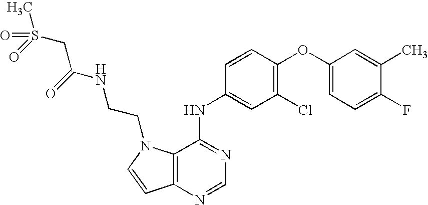

### Production of N-(2-(4-((3-chloro-4-(4-fluoro-3-methylphenoxy)phenyl)amino)-5H-pyrrolo[3,2-d]pyrimidin-5-yl)ethyl)-2-(methylsulfonyl)acetamide

A mixture of tert-butyl[2-(4-chloro-5H-pyrrolo[3,2-d]pyrimidin-5-yl)ethyl]carbamate (1.00 g), 3-chloro-4-(4-fluoro-3-methylphenoxy)aniline (1.51 g) and isopropyl alcohol (10 mL) was stirred at 80° C. for 12 hr. Aqueous sodium bicarbonate was added to the reaction mixture under ice-cooling and the mixture was extracted with ethyl acetate. The organic layer was washed with brine and dried over anhydrous magnesium sulfate. The residue was separated and purified by silica gel column chromatography (eluent, ethyl acetate:hexane=60:40→100:0) to give a crude product (1.52 g). The obtained crude product (150 mg) was dissolved in, tetrahydrofuran (22.2 mL). 4N Hydrogen chloride/ethyl acetate solution (11.5 mL) was added, and the mixture was stirred at 70° C. for 20 hr. The solvent was evaporated under reduced pressure, ethanol was added, and the mixture was further concentrated. Diisopropyl ether was added, and the precipitated powder was collected by filtration. A mixture of the obtained powder, methylsulfonylacetic acid (74 mg), 1-ethyl-3-(3-dimethylaminopropyl)carbodiimide hydrochloride (103 mg), 1-hydroxybenzotriazole (72 mg), triethylamine (0.15 mL) and N,N-dimethylformamide (7.0 mL) was stirred at room temperature for 16 hr. Water was added to the reaction mixture and the mixture was extracted with ethyl acetate. The organic layer was washed successively with water and saturated brine and dried over magnesium sulfate. After concentration under reduced pressure, the residue was separated and purified by basic silica gel column chromatography (eluent, ethyl acetate-Methyl acetate:methanol=90:10) and crystallized from diisopropyl ether to give the title compound (116 mg) as colorless crystals.

¹H-NMR (DMSO-d₆) δ: 2.22 (3H, s), 3.10 (3H, s), 3.46 (2H, q, J=6.0 Hz), 4.04 (2H, s), 4.55 (2H, t, J=6.0 Hz), 6.49-7.17 (5H, m), 7.61-7.93 (3H, m), 8.33 (1H, s), 8.65-8.66 (2H, m).

### Example B-1

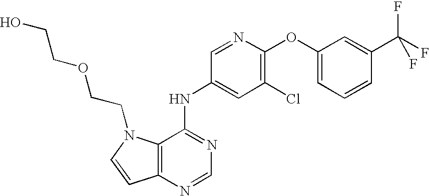

### Production of 2-{2-[4-({5-chloro-6-[3-(trifluoromethyl)phenoxy]pyridin-3-yl}amino)-5H-pyrrolo[3,2-d]pyrimidin-5-yl]ethoxy}ethanol

### (i) Production of 3-chloro-5-nitro-2-[3-(trifluoromethyl)phenoxy]pyridine

Under an argon atmosphere, to a solution of 3-(trifluoromethyl)phenol (0.42 g) in tetrahydrofuran (8.0 mL) was added sodium hydride (60% dispersion in mineral oil, 0.11 g) under ice-cooling. After stirring under ice-cooling for 1 hr, 2,3-dichloro-5-nitropyridine (0.50 g) was added. After stirring at room temperature for 2.5 hr, water was added to the reaction mixture and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=9:1→3:1) to give the title compound (746 mg) as a colorless oil.

¹H-NMR (CDCl₃) δ: 7.35-7.43 (1H, m), 7.45-7.51 (1H, m), 7.55-7.65 (1H, m), 8.61 (1H, d, J=2.7 Hz), 8.88 (1H, d, J=2.7 Hz).

### (ii) Production of 5-chloro-6-[3-(trifluoromethyl)phenoxy]pyridin-3-amine

A mixture of 3-chloro⁻5-nitro-2-[3-(trifluoromethyl)phenoxy]pyridine (746 mg), reduced iron (0.65 g), calcium chloride (0.13 g) and 15% water-containing ethanol (23 mL) was stirred at 80° C. for 8 hr. The solid was removed by filtration, and the filtrate was concentrated under reduced pressure. Water was added to the residue, and the mixture was extracted with ethyl acetate. The organic layer was washed with saturated lo brine and dried over magnesium sulfate. After concentration under reduced pressure, the residue was separated and purified by silica gel column chromatography (eluent, hexane:ethyl acetate=4:1→41:1) to give the title compound (290 mg) as a brown oil.

¹H-NMR (CDCl₃) δ: 3.65 (2H, br s), 7.20 (1H, d, J=2.9 Hz), 7.22-7.26 (1H, m), 7.27-7.32 (1H, m), 7.37-7.40 (1H, m), 7.44-7.50 (1H, m), 7.59 (1H, d, J=2.9 Hz).

### (iii) Production of 2-{2-[4-({5-chloro-6-[3-(trifluoromethyl)phenoxy]pyridin-3-yl}amino)-5H-pyrrolo[3,2-d]pyrimidin-5-yl]ethoxy}ethanol

A solution of 2-[2-(4-chloro-5H-pyrrolo[3,2-d]pyrimidin-5-yl)ethoxy]ethyl benzoate (100 mg) and 5-chloro-6-[3-(trifluoromethyl)phenoxy]pyridin-3-amine (100 mg) in isopropyl alcohol (2.0 mL) was stirred at 80° C. for 16 hr. Aqueous sodium bicarbonate was added to the reaction mixture, and the mixture was extracted with ethyl acetate. The organic layer was washed with saturated brine and dried over magnesium sulfate. After concentration under reduced pressure, the residue was separated and purified by silica gel column chromatography (eluent, ethyl acetate:hexane=1:1→ethyl acetate) to give 2-{2-[4-({5-chloro-6-[3-(trifluoromethyl)phenoxy]pyridin-3-yl)amino)-5H-pyrrolo[3,2-d]pyrimidin-5-yl]ethoxy}ethyl benzoate (130 mg) as a colorless amorphous. To a solution of 2-(2-[4-({5-chloro-6-[3-(trifluoromethyl)phenoxy]pyridin-3-yl}amino)-5H-pyrrolo[3,2-d]pyrimidin-5-yl]ethoxy}ethyl benzoate (130 mg) in isopropyl alcohol-tetrahydrofuran (3 mL-2 mL) was added 1N aqueous sodium hydroxide solution (0.5 mL) at room temperature and the mixture was stirred for 3 hr. The reaction mixture was diluted with ethyl acetate, and the organic layer was washed with saturated brine and dried over magnesium sulfate. After concentration under reduced pressure, the residue was separated and purified by silica gel column chromatography (eluent, ethyl acetate→ethyl acetate:methanol=9:1) to give the title compound (72 mg) as colorless crystals.

¹H-NMR (CDCl₃) δ: 3.69-3.80 (4H, m), 4.00-4.04 (2H, m), 4.54-4.59 (2H, m), 6.65 (1H, d, J=3.3 Hz), 7.23 (1H, d, J=3.3 Hz), 7.31-7.36 (1H, m), 7.40-7.55 (3H, m), 8.24 (1H, d, J=2.7 Hz), 8.47 (1H, J=2.7 Hz), 8.51 (1H, s), 8.83 (1H, s).

### Example B-2

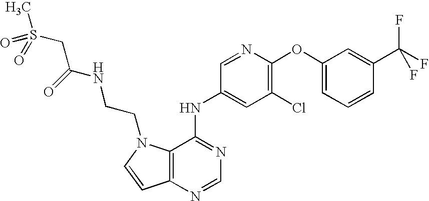

### Production of N-{2-[4-({5-chloro-6-[3-(trifluoromethyl)phenoxy]pyridin-3-yl}amino)-5H-pyrrolo[3,2-d]pyrimidin-5-yl]ethyl}-2-(methylsulfonyl) acetamide

### (i) Production of tert-butyl{2-[4-({5-chloro-6-[3-(trifluoromethyl)phenoxy]pyridin-3-yl}amino)-5H-pyrrolo[3,2-d]pyrimidin-5-yl]ethyl}carbamate

A solution of tert-butyl[2-(4-chloro-5H-pyrrolo[3,2-d]pyrimidin-5-yl)ethyl]carbamate (189 mg) and 5-chloro-6-[3-(trifluoromethyl)phenoxy]pyridin-3-amine (184 mg) in isopropyl alcohol (4.0 mL) was stirred at 80° C. for 20 hr. Aqueous sodium bicarbonate was added to the reaction mixture and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=1:1→ethyl acetate) to give the title compound (257 mg) as a pale-yellow solid.

¹H-NMR (CDCl₃) δ: 1.49 (9H, s), 3.43-4.54 (2H, m), 4.40-4.51 (2H, m), 5.05-5.15 (1H, m), 6.60 (1H, d, J=3.0 Hz), 7.19 (1H, d, J=3.0 Hz), 7.33-7.39 (1H, m), 7.41-7.53 (3H, m), 8.39 (1H, d, J=2.4 Hz), 8.47 (1H, s), 8.64 (1H, d, J=2.4 Hz), 8.79 (1H, s).

### (ii) Production of 5-(2-aminoethyl)-N-{5-chloro-6-[3-(trifluoromethyl)phenoxy]pyridin-3-yl}-5H-pyrrolo[3,2-d]pyrimidin-4-amine trihydrochloride

To a solution of tert-butyl{2-[4-({5-chloro-6-[3-(trifluoromethyl)phenoxy]pyridin-3-yl}amino)-5H-pyrrolo[3,2-d]pyrimidin-5-yl]ethyl}carbamate (257 mg) in tetrahydrofuran (10 mL) was added 2N hydrochloric acid (5.0 mL) at room temperature, and the mixture was stirred at 60° C. for 20 hr. After concentration under reduced pressure, ethanol was added to the residue, and the mixture was concentrated again. Precipitated solid was collected by filtration and the solid was washed with diisopropyl ether to give the title compound (220 mg) as a pale-yellow solid.

¹H-NMR (DMSO-d₆) δ: 3.23-3.37 (2H, m), 4.95-5.08 (2H, m), 6.74 (1H, d, J=2.7 Hz), 7.56 (1H, d, J=8.4 Hz), 7.64-7.74 (3H, m), 8.06 (1H, br s), 8.23-8.45 (5H, m), 8.71 (1H, s), 10.15 (1H, br s).

### (iii) Production of N-{2-[4-({5-chloro-6-[3-(trifluoromethyl)phenoxy]pyridin-3-yl}amino)-5H-pyrrolo[3,2-d]pyrimidin-5-yl]ethyl}-2-(methylsulfonyl)acetamide

A mixture of 5-(2-aminoethyl)-N-{5-chloro-6-[3-(trifluoromethyl)phenoxy]pyridin-3-yl}-5H-pyrrolo[3,2-d]pyrimidin-4-amine trihydrochloride (95 mg), methylsulfonylacetic acid (47 mg), 1-ethyl-3-(3-dimethylaminopropyl)carbodiimide hydrochloride (98 mg), 1-hydroxybenzotriazole (78 mg) and triethylamine (0.12 mL) in N,N-dimethylformamide (5.0 mL) was stirred at room temperature for 14 hr. Water was added to the reaction mixture and the mixture was extracted with ethyl acetate. The organic layer was washed successively with water and saturated brine, and dried over magnesium sulfate. After concentration under reduced pressure, the residue was separated and purified by basic silica gel column chromatography (eluent, ethyl acetate-ethyl acetate:methanol=85:15) to give the title compound (86 mg) as colorless crystals.

¹H-NMR (CDCl₃) δ: 3.10 (3H, s), 3.62-3.78 (2H, m), 3.98 (2H, s), 4.41-4.53 (2H, m), 6.63 (1H, d, J=3.0 Hz), 7.21 (1H, d, J=3.0 Hz), 7.29-7.55 (5H, m), 8.41-8.50 (4H, m).

### Example B-3

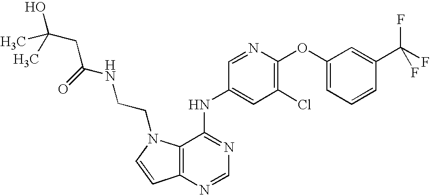

### Production of N-{2-[4-({5-chloro-6-[3-(trifluoromethyl)phenoxy]pyridin-3-yl}amino)-5H-pyrrolo[3,2-d]pyrimidin-5-yl]ethyl}-3-hydroxy-3-methylbutanamide

Using 5-(2-aminoethyl)-N-{5-chloro-6-[3-(trifluoromethyl)phenoxy]pyridin-3-yl}-5H-pyrrolo[3,2-d]pyrimidin-4-amine trihydrochloride (95 mg), 3-hydroxy-3-methylbutanoic acid (46 mg), 1-ethyl-3-(3-dimethylaminopropyl)carbodiimide hydrochloride (98 mg), 1-hydroxybenzotriazole (78 mg), triethylamine (0.12 mL) and N,N-dimethylformamide (5.0 mL) and in the same manner as in Example B-2(iii), the title compound (73 mg) was obtained as colorless crystals.

¹H-NMR (CDCl₃) δ: 1.33 (6H, s), 2.36-2.43 (1H, m), 2.48 (2H, s), 3.55-3.66 (2H, m), 4.41-4.50 (2H, m), 6.60 (1H, d, J=3.0 Hz), 7.18-7.22 (2H, m), 7.34-7.39 (1H, m), 7.42-7.53 (3H, m), 8.44 (1H, d, J=2.4 Hz), 8.47 (1H, s), 8.54 (1H, d, J=2.4 Hz), 8.97 (1H, s).

### Example B-4

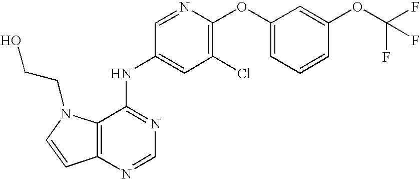

### Production of 2-[4-({5-chloro-6-[3-(trifluoromethoxy)phenoxy]pyridin-3-yl}amino)-5H-pyrrolo[3,2-d]pyrimidin-5-yl]ethanol

### (i) Production of 3-chloro-5-nitro-2-[3-(trifluoromethoxy)phenoxy]pyridine

Under an argon atmosphere, using 3-(trifluoromethoxy)phenol (0.93 g), 2,3-dichloro-5-nitropyridine (1.0 g), sodium hydride (60% dispersion in mineral oil, 0.23 g) and tetrahydrofuran (10 mL) and in the same manner as in Example B-1(i), the title compound (1.57 g) was obtained as a pale-yellow oil.

¹H-NMR (CDCl₃) δ: 7.06-7.22 (3H, m), 7.49 (1H, t, J=8.3 Hz), 8.59 (1H, d, J=2.4 Hz), 8.88 (1H, d, J=2.4 Hz).

### (ii) Production of 5-chloro-6-[3-(trifluoromethoxy)phenoxy]pyridin-3-amine

Using 3-chloro-5-nitro-2-[3-(trifluoromethoxy)phenoxy]pyridine (1.57g), reduced iron (1.31 g), calcium chloride (0.26 g), and 15% water-containing ethanol (50 mL) and in the same manner as in Example B-1(ii), the title compound (1.23 g) was obtained as an orange oil.

¹H-NMR (CDCl₃) δ: 3.65 (2H, br s), 6.91-7.02 (3H, m), 7.18 (1H, d, J=2.7 Hz), 7.35 (1H, t, J=8.1 Hz), 7.59 (1H, d, J=2.7′Hz).

### (iii) Production of 2-[4-({5-chloro-6-[3-(trifluoromethoxy)phenoxy]pyridin-3-yl}amino)-5H-pyrrolo[3,2-d]pyrimidin-5-yl]ethanol

A solution of 2-(4-chloro-5H-pyrrolo[3,2-d]pyrimidin-5-yl)ethyl benzoate (100 mg) and 5-chloro-6-[3-(trifluoromethoxy)phenoxy]pyridin-3-amine (101 mg) in isopropyl alcohol (2.0 mL) was stirred at 80° C. for 2 days. The reaction mixture was cooled to room temperature, 1N aqueous sodium hydroxide solution (1.0 mL) was added thereto. The reaction mixture was stirred at room temperature for 4 hr, water was added thereto, and the mixture was extracted with ethyl acetate. The organic layer was washed successively with water and saturated brine, and dried over magnesium sulfate. After concentration under reduced pressure, the residue was separated and purified by column chromatography (eluent, ethyl acetate→ethyl acetate:methanol=9:1) to give the title compound (112 mg) as colorless crystals.

¹H-NMR (CDCl₃) δ: 4.11-4.19 (2H, m), 4.39-4.45 (2H, m), 4.83-4.99 (1H, m), 6.31 (1H, d, J=3.3 Hz), 7.02-7.10 (4H, m), 7.36-7.42 (1H, m), 8.17 (1H, d, J=2.7 Hz), 8.31 (1H, s), 8.34 (1H, d, J=2.7 Hz), 9.44 (1H, s).

### Example B-5

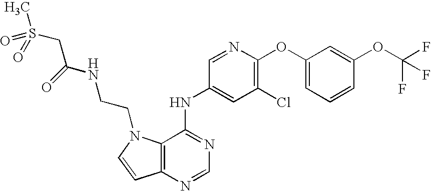

### Production of N-{2-[4-({5-chloro-6-[3-(trifluoromethoxy)phenoxy]pyridin-3-yl}amino)-5H-pyrrolo[3,2-d]pyrimidin-5-yl]ethyl}-2-(methylsulfonyl)acetamide

### (i) Production of tert-butyl{2-[4-({5-chloro-6-[3-(trifluoromethoxy)phenoxy]pyridin-3-yl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (300 mg), 5-chloro-6-[3-(trifluoromethoxy)phenoxy]pyridin-3-amine (308 mg) and isopropyl alcohol (3.0 mL) and in the same manner as in Example B-2(i), the title compound (372 mg) was obtained as colorless crystals.

¹H-NMR (CDCl₃) δ: 1.49 (9H, s), 3.45-3.53 (2H; m), 4.43-4.49 (2H, m), 5.10 (1H, t, J=5.4 Hz), 6.60 (1H, d, J=3.0 Hz), 7.02-7.12 (3H, m), 7.18 (1H, d, J=3.0 Hz), 7.36-7.42 (1H, m), 8.38 (1H, d, J=2.4 Hz), 8.47 (1H, s), 8.65 (1H, d, J=2.4 Hz), 8.77 (1H, br s).

### (ii) Production of 5-(2-aminoethyl)-N-{5-chloro-6-[3-(trifluoromethoxy)phenoxy]pyridin-3-yl}-5H-pyrrolo[3,2-d]pyrimidin-4-amine trihydrochloride

Using tert-butyl{2-[4-({5-chloro-6-[3-(trifluoromethoxy)phenoxy]pyridin-3-yl}amino)-5H-pyrrolo[3,2-d]pyrimidin-5-yl]ethyl}carbamate (350 mg), 2N hydrochloric acid (5.0 mL) and tetrahydrofuran (10 mL) and in the same manner as in Example B-2(ii), the title compound (294 mg) was obtained as pale-yellow crystals.

¹H-NMR (DMSO-d₆) δ: 3.20-3.34 (2H, m), 4.91-5.03 (2H, m), 6.66-6.76 (1H, m), 7.20-7.32 (3H, m), 7.59 (1H, t, J=8.1 Hz), 8.01 (1H, br s), 8.12-8.37 (5H, m), 8.68 (1H, br s), 9.94-10.06 (1H, m).

### (iii) Production of N-{2-[4-({5-chloro-6-[3-(trifluoromethoxy)phenoxy]pyridin-3-yl}amino)-5H-pyrrolo[3,2-d]pyrimidin-5-yl]ethyl}-2-(methylsulfonyl)acetamide

Using 5-(2-aminoethyl)-N-{5-chloro-6-[3-(trifluoromethoxy)phenoxy]pyridin-3-yl}-5H-pyrrolo[3,2-d]pyrimidin-4-amine trihydrochloride (90 mg), methylsulfonylacetic acid (43 mg), 1-ethyl-3-(3-dimethylaminopropyl)carbodiimide hydrochloride (90 mg), 1-hydroxybenzotriazole monohydrate (72 mg), triethylamine (0.12 mL) and N,N-dimethylformamide (5.0 mL) and in the same manner as in Example B-2(iii), the title compound (59 mg) was obtained as pale-yellow crystals.

¹H-NMR (CDCl₃) δ: 3.10 (3H, s), 3.64-3.75 (2H, m), 3.98 (2H, s), 4.43-4.53 (2H, m), 6.62 (1H, d, J=3.0 Hz), 7.03-7.13 (3H, m), 7.15-7.23 (2H, m), 7.41 (1H, t, J=8.4 Hz), 8.42 (1H, s), 8.44-8.47 (2H, m), 8.49 (1H, s).

### Example B-6

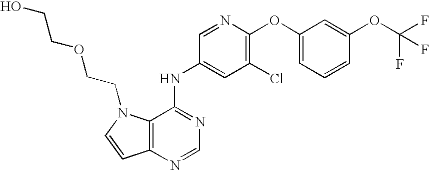

### Production of 2-{2-[4-({5-chloro-6-[3-(trifluoromethoxy)phenoxy]pyridin-3-yl}amino)-5H-pyrrolo[3,2-d]pyrimidin-5-yl]ethoxy}ethanol

Using 2-[2-(4-chloro-5H-pyrrolo[3,2-d]pyrimidin-5-yl)ethoxy]ethyl benzoate (100 mg), 5-chloro-6-[3-(trifluoromethoxy)phenoxy]pyridin-3-amine (80 mg), isopropyl alcohol (2.0 mL) and 1N aqueous sodium hydroxide solution (1.0 mL) and in the same manner as in Example B-4(iii), the title compound (71 mg) was obtained as pale-yellow crystals.

¹H-NMR (CDCl₃) δ: 1.77 (1H, br s), 3.66-3.80 (4H, m), 4.01 (2H, t, J=4.5 Hz), 4.56 (2H, t, J=4.5 Hz), 6.64 (1H, d, J=3.3 Hz), 7.01-7.09 (3H, m), 7.22 (1H, d, J=3.3 Hz), 7.36-7.42 (1H, m), 8.25 (1H, d, J=2.7 Hz), 8.47 (1H, d, J=2.7 Hz), 8.49 (1H, s), 8.83 (1H, s).

### Example B-7

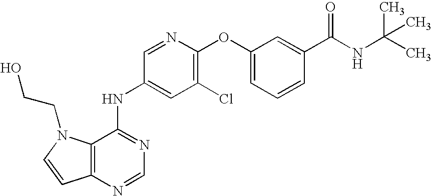

### Production of N-(tert-butyl)-3-[(3-chloro-5-{[5-(2-hydroxyethyl)-5H-pyrrolo[3,2-d]pyrimidin-4-yl]amino}pyridin-2-yl)oxy]benzamide

### (i) Production of methyl 3-[(3-chloro-5-nitropyridin-2-yl)oxy]benzoate

Using methyl 3-hydroxybenzoate (0.83 g), 2,3-dichloro-5-nitropyridine (1.0 g), sodium hydride (60% dispersion in mineral oil, 0.24 g) and tetrahydrofuran (10 mL) and in the same manner as in Example B-1(i), the title compound (1.61 g) was obtained as a colorless oil.

¹H-NMR (CDCl₃) δ: 3.93 (3H, s), 7.37-7.41 (1H, m), 7.52-7.57 (1H, m), 7.84-7.86 (1H, m), 7.98-8.02 (1H, m), 8.58 (1H, d, J=2.7 Hz), 8.86 (1H, d, J=2.7 Hz).

### (ii) Production of 3-[(3-chloro-5-nitropyridin-2-yl)oxy]benzoic acid

To a solution of methyl 3-[(3-chloro-5-nitropyridin-2-yl)oxy]benzoate (1.61 g) in isopropyl alcohol (20 mL) and tetrahydrofuran (10 mL) was added 1N aqueous sodium hydroxide solution (6.0 mL) at room temperature. After stirring at room temperature for 24 hr, 1N hydrochloric acid (6.0 mL) was added to the reaction mixture and the mixture was extracted with ethyl acetate. The organic layer was washed with saturated brine and dried over magnesium sulfate. After concentration under reduced pressure, the resulting crystals were collected by filtration. The crystals were washed with diisopropyl ether to give the title compound (0.62 g) as colorless crystals.

¹H-NMR (CDCl₃) δ: 7.42-7.48 (2H, m), 7.57-7.63 (1H, m), 7.90-7.94 (1H, m), 8.06-8.08 (1H, m), 8.60-8.61 (1H, m), 8.88 (1H, d, J=2.4 Hz).

### iii) Production of N-(tert-butyl)-3-[(3-chloro-5-nitropyridin-2-yl)oxy]benzamide

To a solution of 3-[(3-chloro-5-nitropyridin-2-yl)oxy]benzoic acid (0.62 g) and N,N-dimethylformamide (0.1 mL) in tetrahydrofuran (12 mL) was added thionyl chloride (0.23 mL) at room temperature. After stirring at room temperature for 2 hr, the mixture was concentrated under reduced pressure. A solution of the residue in tetrahydrofuran (10 mL) was added dropwise to a solution of tert-butylamine (0.3 g) and triethylamine (0.89 mL) in tetrahydrofuran (5.0 mL) at 0° C. After stirring at room temperature for 20 hr, water was added to the reaction mixture and the mixture was extracted with ethyl acetate. The organic layer was washed successively with water and saturated brine, and dried over magnesium sulfate. After concentration under reduced pressure, the residue was separated and purified by column chromatography (eluent, hexane:ethyl acetate=9:1→2:1) to give the title compound (0.61 g) as a pale-yellow solid.

¹H-NMR (CDCl₃) δ: 1.47 (9H, s), 5.93 (1H, br s), 7.28-7.32 (1H, m), 7.52 (1H, t, J=8.0 Hz), 7.57 (1H, t, J=2.1 Hz), 7.62-7.65 (1H, m), 8.59 (1H, d, J=2.4 Hz), 8.87 (1H, d, J=2.4 Hz).

### (iv) Production of 3-[(5-amino-3-chloropyridin-2-yl)oxy]-N-(tert-butyl)benzamide

Using N-(tert-butyl)-3-[(3-chloro-5-nitropyridin-2-yl)oxy]benzamide (570 mg), reduced iron (0.46 g), calcium chloride (90 mg) and 15% water-containing ethanol (17 mL) and in the same manner as in Example B-1(ii), the title compound (373 mg) was obtained as pale-yellow crystals.

¹H-NMR (CDCl₃) δ: 1.45 (9H, s), 3.63 (2H, br s), 5.91 (1H, br s), 7.15-7.19 (2H, m), 7.36-7.47 (3H, m), 7.56 (1H, d, J=2.7 Hz).

### (v) Production of N-(tert-butyl)-3-[(3-chloro-5-{[5-(2-hydroxyethyl)-5H-pyrrolo[3,2-d]pyrimidin-4-yl]amino}pyridin-2-yl)oxy]benzamide

Using 2-(4-chloro-5H-pyrrolo[3,2-d]pyrimidin-5-yl)ethyl benzoate (80 mg), 3-[(5-amino-3-chloropyridin-2-yl)oxy]-N-(tert-butyl)benzamide (85 mg), isopropyl alcohol (2.0 mL) and 1N aqueous sodium hydroxide solution (1.0 mL) and in the same manner as in. Example B-4(iii), the title compound. (78 mg) was obtained as colorless crystals.

¹H-NMR (CDCl₃) δ: 1.49 (9H, s), 4.11 (2H, t, J=4.5 Hz), 4.41 (2H, t, J=4.5 Hz), 5.44-5.56 (1H, m), 5.98 (1H, s), 6.30 (1H, d, J=3.0 Hz), 7.06 (1H, d, J=3.0 Hz), 7.20-7.28 (1H, m), 7.37-7.43 (1H, m), 7.46-7.50 (2H, m), 8.09 (1H, d, J=2.7 Hz), 8.28 (1H, s), 8.31 (1H, d, J=2.7 Hz), 9.57 (1H, s).

### Example C-1

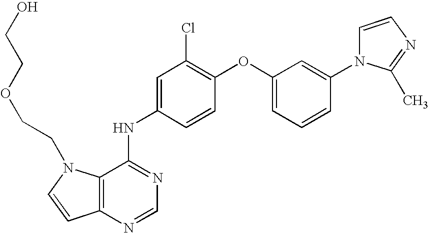

### Production of 2-{2-[4-({3-chloro-4-[3-(2-methyl-1H-imidazol-1-yl)phenoxy]phenyl}amino)-5H-pyrrolo[3,2-d]pyrimidin-5-yl]ethoxy}ethanol

### (i) Production of 1-[3-(2-chloro-4-nitrophenoxy)phenyl]-2-methyl-1H-imidazole

To a solution of 3-(2-methyl-1H-imidazol-1-yl)phenol (1.10 g) and 3-chloro-4-fluoronitrobenzene (1.28 g) in N,N-dimethylformamide (10 mL) was added potassium carbonate (1.31 g) and the mixture was stirred at room temperature for 18 hr. Brine was added to the reaction mixture under ice-cooling, and the mixture was extracted with ethyl acetate twice, and the organic layer was dried over anhydrous magnesium sulfate. After concentration under reduced pressure, the residue was separated and purified by silica gel column chromatography (eluent, ethyl acetate:methanol=100:0→95:5) to give the title compound (1.86 g) as a pale-yellow oil.

¹H-NMR (CDCl₃) δ: 2.40 (3H, s), 7.00-7.25 (6H, m), 7.54 (1H, t, J=8.2 Hz), 8.12 (1H, dd, J=2.7, 9.0 Hz), 8.41 (1H, d, J=2.4 Hz).

### (ii) Production of 3-chloro-4-[3-(2-methyl-1H-imidazol-1-yl)phenoxy]aniline

To a solution of 1-[3-(2-chloro-4-nitrophenoxy)phenyl]-2-methyl-1H-imidazole (1.86 g) in ethyl acetate (30 mL)/methanol (2 mL) was added 5% platinum-activated carbon (0.37 g) under a nitrogen atmosphere. The reaction mixture was stirred under a hydrogen atmosphere at room temperature for 3.5 hr, the platinum-activated carbon was filtered off, and the filtrate was concentrated under reduced pressure. The residue was separated and purified by basic silica gel column chromatography (eluent, ethyl acetate:hexane=60:40→100:0) to give the title compound (1.26 g) as colorless crystals.

¹H-NMR (CDCl₃) δ: 2.34 (3H, s), 3.73 (2H, br s), 6.58 (1H, dd, J=2.7, 8.7 Hz), 6.74 (1H, t, J=2.1 Hz), 6.79 (1H, d, J=2.4 Hz), 6.9-7.05 (5H, m), 7.37 (1H, t, J=8.1 Hz).

### (iii) Production of 2-{2-[4-({3-chloro-4-[3-(2-methyl-1H-imidazol-1-yl)phenoxy]phenyl}amino)-5H-pyrrolo[3,2-d]pyrimidin-5-yl]ethoxy}ethanol

A mixture of 2-[2-(4-chloro-5H-pyrrolo[3,2-d]pyrimidin-5-yl)ethoxy]ethyl benzoate (207 mg), 3-chloro-4-[3-(2-methyl-1H-imidazol-1-yl)phenoxy]aniline (180 mg), 1-methyl-2-pyrrolidone (4.0 mL) and pyridine hydrochloride (139 mg) was stirred, at 120° C. for 17 hr. Aqueous sodium bicarbonate was added to the reaction mixture and the mixture was extracted with ethyl acetate. The organic layer was washed with brine and dried over anhydrous magnesium sulfate. After concentration under reduced pressure, the residue was separated and purified by basic silica gel column chromatography (eluent, ethyl acetate:methanol=100:0→95:5). To the obtained compound were added 1N aqueous sodium hydroxide solution (2.6 mL) and tetrahydrofuran (5 mL) and the mixture was stirred at room temperature for 3 days. The reaction mixture was neutralized with 1N hydrochloric acid, and aqueous sodium bicarbonate and brine were added. The mixture was extracted with ethyl acetate and dried over anhydrous magnesium sulfate. After concentration under reduced pressure, the residue was separated and purified by basic silica gel column chromatography (eluent, ethyl acetate:methanol=100:0→95:5) and the obtained solid was collected by filtration and washed with diisopropyl ether to give the title compound (158 mg) as a white powder.

¹H-NMR (CDCl₃) δ: 2.35 (3H, s), 3.70-3.75 (2H, m), 3.75-3.85 (2H, m), 4.02 (2H, t, J=4.4 Hz), 4.57 (2H, t, J=4.4 Hz), 6.64 (1H, d, J=3.0 Hz), 6.80-6.85 (1H, m), 6.95-7.05 (4H, m), 7.11 (2H, d, J=9.0 Hz), 7.22 (1H, d, J=3.6 Hz), 7.40 (1H, t, J=8.4 Hz), 7.64 (1H, dd, J=2.4, 9.0 Hz), 7.90 (1H, d, J=2.4 Hz), 8.53 (1H, s), 8.82 (1H, s).

### Example C-2

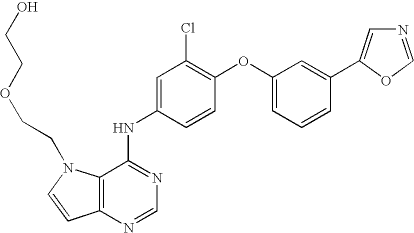

### Production of 2-{2-[4-({3-chloro-4-[3-(1,3-oxazol-5-yl)phenoxy]phenyl}amino)-5H-pyrrolo[3,2-d]pyrimidin-5-yl]ethoxy}ethanol

### (i) Production of 5-[3-(benzyloxy)phenyl]-1,3-oxazole

To a solution of 3-(benzyloxy)benzaldehyde (2.12 g) and p-toluenesulfonylmethyl isocyanide (1.95 g) in methanol (40 mL) was added potassium carbonate (1.66 g) under ice-cooling, and the mixture was stirred at room temperature for 20 min and refluxed for 1 hr. After concentration under reduced pressure, water was added, and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:hexane=20:80→50:50) to give the title compound (2.04 g) as a white powder.

¹H-NMR (CDCl₃) δ: 5.12 (2H, s), 6.90-7.00 (1H, m), 7.25-7.50 (9H, m), 7.91 (1H, s).

### (ii) Production of 3-(1,3-oxazol-5-yl)phenol

To a solution of 5-[3-(benzyloxy)phenyl]-1,3-oxazole (2.01 g) in methanol (10 mL)/tetrahydrofuran (10 mL) was added 10% palladium-activated carbon (0.40 g) and the mixture was stirred under a hydrogen atmosphere at room temperature for 5 hr. The palladium-activated carbon was filtered off, and the filtrate was concentrated under reduced pressure. The precipitate was washed with diisopropyl ether and hexane to give the title compound (1.25 g) as pale-gray crystals.

¹H-NMR (95% CDCl₃+5% DMSO-d₆) δ: 6.80-6.90 (1H, m), 7.10-7.20 (2H, m), 7.24 (1H, t, J=8.0 Hz), 7.31 (1H, s), 7.94 (1H, s), 9.13 (1H, s).

### (iii) Production of 5-[3-(2-chloro-4-nitrophenoxy)phenyl]-1,3-oxazole

To a solution of 3-(1,3-oxazol-5-yl)phenol (1.20 g) and 3-chloro-4-fluoronitrobenzene (1.45 g) in N,N-dimethylformamide (10 mL) was added potassium carbonate (1.54 g) and the mixture was stirred at room temperature for 18 hr. To the reaction mixture was added brine under ice-cooling, and the mixture was extracted with ethyl acetate, and the organic layer was dried over anhydrous magnesium sulfate. After concentration under reduced pressure, the residue was recrystallized from ethyl acetate/diisopropyl ether/hexane to give the title compound (2.00 g) as pale-yellow crystals.

¹H-NMR (CDCl₃) δ: 6.96 (1H, t, J=9.0 Hz), 7.00-7.10 (1H, m), 7.35-7.45 (2H, m), 7.45-7.60 (2H, m), 7.93 (1H, s), 8.08 (1H, dd, J=3.0 Hz, 9.0 Hz), 8.40 (1H, d, J=3.0 Hz).

### (iv) Production of 3-chloro-4-[3-(1,3-oxazol-5-yl)phenoxy]aniline

Using 5-[3-(2-chloro-4-nitrophenoxy)phenyl]-1,3-oxazole (1.95 g), 5% platinum-activated carbon (0.33 g) and ethyl acetate (30 mL)/methanol (5 mL) and in the same manner as in Example C-1(ii), the title compound (1.80 g) was obtained as pale-yellow crystals.

¹H-NMR (95% CDCl₃+5% DMSO-d₆) δ: 6.8-6.9 (2H, m), 6.95-7.05 (2H, m), 7.15-7.2 (2H, m), 7.3-7.4 (3H, m), 7.91 (1H, s), 8.09 (1H, s).

### (v) Production of 2-{2-[4-({3-chloro-4-[3-(1,3-oxazol-5-yl)phenoxy]phenyl}amino)-5H-pyrrolo[3,2-d]pyrimidin-5-yl]ethoxy}ethanol

A mixture of 2-[2-(4-chloro-5H-pyrrolo[3,2-d]pyrimidin-5-yl)ethoxy]ethyl benzoate (346 mg), 3-chloro-4-[3-(1,3-oxazol-5-yl)phenoxy]aniline (344 mg) and isopropyl alcohol (10 mL) was stirred at 80° C. for 18 hr. Aqueous sodium bicarbonate was added to the reaction mixture under ice-cooling and the mixture was extracted with ethyl acetate. The organic layer was washed with brine and dried over anhydrous magnesium sulfate. The residue was separated and purified by silica gel column chromatography (eluent, ethyl acetate:methanol=100:0→95:5). 1N Aqueous sodium hydroxide solution (0.8 mL) and tetrahydrofuran (4.0 mL) were added to the obtained compound, and the mixture was stirred at room temperature for 2 days. The reaction mixture was neutralized with 1N hydrochloric acid, and aqueous sodium bicarbonate and brine were added. The mixture was extracted with ethyl acetate, and the extract was dried over anhydrous magnesium sulfate. After concentration under reduced pressure, the residue was separated and purified by silica gel column chromatography (eluent, ethyl acetate:methanol=100:0→95:5) to give the title compound (26 mg) as a pale-yellow powder.

¹H-NMR (CDCl₃) δ: 3.70-3.75 (2H, m), 3.75-3.85 (2H, m), 4.03 (2H, t, J=4.5 Hz), 4.58 (2H, t, J=4.5 Hz), 6.64 (1H, d, J=3.0 Hz), 6.90-6.95 (1H, m), 7.08 (1H, d, J=9.0 Hz), 7.22 (1H, d, J=3.3 Hz), 7.25-7.30 (1H, m), 7.30-7.40 (3H, m), 7.61 (1H, dd, J=2.4 Hz, 9.0 Hz), 7.89 (2H, d, J=2.1 Hz), 8.53 (1H, s), 8.78 (1H, s).

### Example C-3

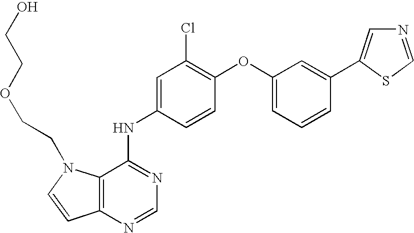

### Production of 2-{2-[4-({3-chloro-4-[3-(1,3-thiazol-5-yl)phenoxy]phenyl}amino)-5H-pyrrolo[3,2-d]pyrimidin-5-yl]ethoxy}ethanol

### (i) Production of 5-[3-(2-chloro-4-nitrophenoxy)phenyl]-1,3-thiazole

Using 3-(1,3-thiazol-5-yl)phenol (343 mg), 3-chloro-4-fluoronitrobenzene (429 mg), potassium carbonate (401 mg) and N,N-dimethylformamide (5.0 mL) and in the same manner as in Example C-1(i), the title compound (624 mg) was obtained as a colorless oil.

¹H-NMR (CDCl₃) δ: 6.96 (1H, t, J=9.3 Hz), 7.00-7.10 (1H, m), 7.30-7.35 (1H, m), 7.50-7.55 (2H, m), 8.07 (1H, d, J=2.7 Hz), 8.10-8.15 (1H, m), 8.41 (1H, d, J=2.4 Hz), 8.79 (1H, d, J=0.6 Hz).

### (ii) Production of 2-{2-[4-({3-chloro-4-[3-(1,3-thiazol-5-yl)phenoxy]phenyl}amino)-5H-pyrrolo[3,2-d]pyrimidin-5-yl]ethoxy}ethanol

A mixture of the compound obtained using 5-[3-(2-chloro-4-nitrophenoxy)phenyl]-1,3-thiazole (624 mg), 5% platinum-activated carbon (312 mg) and ethyl acetate (10 mL) and in the same manner as in Example C-1(ii), 2-[2-(4-chloro-5H-pyrrolo[3,2-d]pyrimidin-5-yl)ethoxy]ethyl benzoate (450 mg) and isopropyl alcohol (10 mL) was stirred at 80° C. for 20 hr. Aqueous sodium bicarbonate was added to the reaction mixture under ice-cooling, and the mixture was extracted with ethyl acetate. The organic layer was washed with brine and dried over anhydrous magnesium sulfate. The residue was separated and purified by silica gel column chromatography (eluent, ethyl acetate:methanol=100:0→95:5). To the obtained compound were added 1N aqueous sodium hydroxide solution (2.2 mL) and tetrahydrofuran (5 mL) and the mixture was stirred at room temperature for 2 days. The) reaction mixture was neutralized with 1N hydrochloric acid, and aqueous sodium bicarbonate and brine were added. The mixture was extracted with ethyl acetate and dried over anhydrous magnesium sulfate. After concentration under reduced pressure, the residue was separated and purified by silica gel column chromatography (eluent, ethyl acetate:methanol=100:0→95:5) and further washed with diisopropyl ether to give the title compound (63.5 mg) as a pale-yellow powder.

¹H-NMR (CDCl₃) δ: 3.70-3.85 (4H, m), 4.00-4.10 (2H, m), 4.50-4.60 (2H, m), 6.64 (1H, d, J=3.0 Hz), 6.85-6.95 (1H, m), 7.08 (1H, d, J=8.7 Hz), 7.20-7.40 (4H, m), 7.61 (1H, dd, J=2.4, 8.7 Hz), 7.90 (1H, d, J=2.4 Hz), 8.06 (1H, s), 8.53 (1H, s), 8.75 (1H, s), 8.78 (1H, s).

### Example C-4

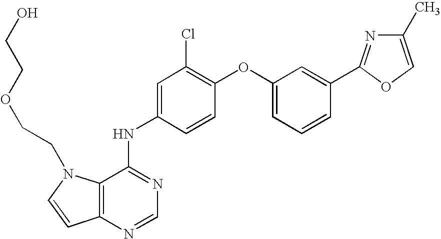

### Production of 2-{2-[4-({3-chloro-4-[3-(4-methyl-1,3-oxazol-2-yl)phenoxy]phenyl}amino)-5H-pyrrolo[3,2-d]pyrimidin-5-yl]ethoxy}ethanol

### (i) Production of 2-(3-methoxyphenyl)-4-methyl-1,3-oxazole

A suspension of 3-methoxybenzamide (4.91 g) and chloroacetone (3.61 g) in toluene (30 mL) was stirred at 110° C. for 2 days. Water was added to the reaction mixture and the mixture was extracted with ethyl acetate. The organic layer was washed successively with aqueous sodium bicarbonate and brine, and dried over anhydrous magnesium sulfate. After concentration under reduced pressure; the residue was separated and purified by silica gel column chromatography (eluent, ethyl acetate:hexane=10:90→30:70) to give the title compound (1.54 g) as a yellow oil.

¹H-NMR (CDCl₃) δ: 2.25 (3H, s), 3.88 (3H, s), 6.95-7.05 (1H, m), 7.35 (1H, t, J=8.0 Hz), 7.40-7.45 (1H, m), 7.50-7.65 (2H, m).

### (ii) Production of 3-(4-methyl-1,3-oxazol-2-yl)phenol

A solution (10 mL) of 2-(3-methoxyphenyl)-4-methyl-1,3-oxazole (1.54 g) in 48% hydrobromic acid was refluxed for 24 hr. Water was added to the reaction mixture and the mixture was extracted with ethyl acetate. The organic layer was washed with aqueous sodium bicarbonate and brine, and dried over anhydrous magnesium sulfate. After concentration under reduced pressure, the residue was separated and purified by silica gel column chromatography (eluent, ethyl acetate:hexane=10:90→40:60) to give the title compound (1.14 g) as a white powder.

¹H-NMR (CDCl₃) δ: 2.24 (3H, s), 6.09 (1H, br s), 6.90-7.00 (1H, m), 7.30 (1H, t, J=8.0 Hz), 7.40-7.45 (1H, m), 7.50-7.60 (2H, m).

### (iii) Production of 2-[3-(2-chloro-4-nitrophenoxy)phenyl]-4-methyl-1,3-oxazole

Using 3-(4-methyl-1,3-oxazol-2-yl)phenol (1.09 g), 3-chloro-4-fluoronitrobenzene (1.21 g), potassium carbonate (1.29 g) and N,N-dimethylformamide (10 mL) and in the same manner as in Example C-1(i), the title compound (1.86 g) was obtained as a pale-yellow oil.

¹H-NMR (CDCl₃) δ: 2.38 (3H, s), 6.94 (1H, t, J=9.2 Hz), 7.10-7.20 (1H, m), 7.40-7.45 (1H, m), 7.53 (1H, t, J=8.0 Hz), 7.70-7.75 (1H, m), 7.85-7.95 (1H, m), 8.07 (1H, dd, J=2.6, 9.2 Hz), 8.40 (1H, d, J=2.6 Hz).

### (iv) Production of 3-chloro-4-[3-(4-methyl-1,3-oxazol-2-yl)phenoxy]aniline

Using 2-[3-(2-chloro-4-nitrophenoxy)phenyl]-4-methyl-1,3-oxazole (1.86 g), 5% platinum-activated carbon (0.31 g) and ethyl acetate (20 mL) and in the same manner as in Example C-1(ii); the title compound (0.41 g) was obtained as a colorless oil.

¹H-NMR (CDCl₃) δ: 2.23 (3H, s), 3.69 (2H, br s), 6.58 (1H, dd, J=2.7 Hz, 9.0 Hz), 6.80 (1H, d, J=3.0 Hz), 6.96 (1H, d, J=6.9 Hz), 6.95-7.00 (1H, m), 7.36 (1H, t, J=8.0 Hz), 7.35-7.40 (1H, m), 7.50-7.55 (1H, m), 7.70-7.75 (1H, m).

### (v) Production of 2-{2-[4-({3-chloro-4-[3-(4-methyl-1,3-oxazol-2-yl)phenoxy]phenyl}amino)-5H-pyrrolo[3,2-d]pyrimidin-5-yl]ethoxy}ethanol

Using 2-[2-(4-chloro-5H-pyrrolo[3,2-d]pyrimidin-5-yl)ethoxy]ethyl benzoate (392 mg), 3-chloro-4-[3-(4-methyl-1,3-oxazol-2-yl)phenoxy]aniline (410 mg) and isopropyl alcohol (10 mL), the reaction was carried out in the same manner as in Example C-2(v). Then, the obtained compound was reacted in the same manner as in Example C-2(v) and using 1N aqueous sodium hydroxide solution (4.7 mL) and tetrahydrofuran (10 mL) to give the title compound (371 mg) as a white powder.

¹H-NMR (CDCl₃) δ: 2.21 (3H, s), 3.65-3.85 (4H, m), 4.02 (2H, t, J=4.4 Hz), 4.57 (2H, t, J=4.4 Hz), 6.62 (1H, d, J=3.2 Hz), 7.05-7.15 (2H, m), 7.20 (1H, d, J=3.0 Hz), 7.30-7.45 (2H, m), 7.50-7.55 (1H, m), 7.62 (1H, dd, J=2.6 Hz, 8.8 Hz), 7.70-7.75 (1H, m), 7.90.(1H, d, J=2.6 Hz), 8.52 (1H, s), 8.79 (1H, s).

### Example C-5

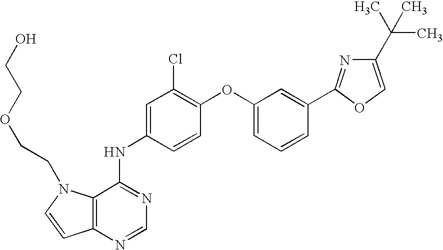

### Production of 2-{2-[4-({4-[3-(4-tert-butyl-1,3-oxazol-2-yl)phenoxy]-3-chlorophenyl}amino)-5H-pyrrolo[3,2-d]pyrimidin-5-yl]ethoxy}ethanol

### (i) Production of 4-tert-butyl-2-(3-methoxyphenyl)-1,3-oxazole

Using 3-methoxybenzamide (1.51 g), 1-bromopinacolone (2.15 g) and toluene (10 mL) and in the lo same manner as in Example C-4(i), the title compound (2.01 g) was obtained as a pale-yellow oil.

¹H-NMR (CDCl₃) δ: 1.32 (9H, s), 3.88 (3H, s), 6.96 (1H, dd, J=2.6 Hz, 8.4 Hz), 7.30-7.40 (2H, m), 7.55-7.65 (2H, m).

### (ii) Production of 3-(4-tert-butyl-1,3-oxazol-2-yl)phenol

Using 4-tert-butyl-2-(3-methoxyphenyl)-1,3-oxazole (2.01 g) and 48% hydrobromic acid (10 mL) and in the same manner, as in Example C-4(ii), the title compound (0.62 g) was obtained as a pale-yellow powder.

¹H-NMR (CDCl₃) δ: 1.31 (9H, s), 5.20-5.50 (1H, m), 6.90 (1H, dd, J=1.8 Hz, 8.0 Hz), 7.31 (1H, d, J=7.6 Hz), 7.36 (1H, s), 7.45-7.55 (1H, m), 7.58 (1H, d, J=7.2 Hz).

### (iii) Production of 4-tert-butyl-2-[3-(2-chloro-4-nitrophenoxy)phenyl]-1,3-oxazole

Using 3-(4-tert-butyl-1,3-oxazol-2-yl)phenol (1.48 g), 3-chloro-4-fluoronitrobenzene (1.26 g), potassium carbonate (1.41 g) and N,N-dimethylformamide (12 mL) and in the same manner as in Example C-1(i), the title compound (2.13 g) was obtained as a white powder.

¹H-NMR (CDCl₃) δ: 1.31 (9H, s), 6.92 (1H, t, J=9.3 Hz), 7.10-7.20 (1H, m), 7.37 (1H, s), 7.51 (1H, t, J=8.1 Hz), 7.75-7.80 (1H, m), 7.94 (1H, t, J=7.8 Hz), 8.06 (1H, dd, J=2.7 Hz, 9.3 Hz), 8.40 (1H, d, J=2.7 Hz).

### (iv) Production of 4-[3-(4-tert-butyl-1,3-oxazol-2-yl)phenoxy]-3-chloroaniline

Using 4-tert-butyl-2-[3-(2-chloro-4-nitrophenoxy)phenyl]-1,3-oxazole (1.12 g), 5% platinum-activated carbon (0.19 g) and ethyl acetate (20 mL)/methanol (4 mL) and in the same manner as in Example C-1(ii), the title compound (985 mg) was obtained as a colorless oil.

¹H-NMR (CDCl₃) δ: 1.30 (9H, s), 3.68 (2H, br s), 6.58 (1H, dd, J=2.6, 8.4 Hz), 6.80 (1H, d, J=2.6 Hz), 6.85-6.95 (2H, m),7.33 (2H, t, J=8.4 Hz), 7.55-7.60 (1H, m), 7.70-7.75 (1H, m).

### (v) Production of 2-{2-[4-({4-[3-(4-tert-butyl-1,3-oxazol-2-yl)phenoxy]-3-chlorophenyl}amino)-5H-pyrrolo[3,2-d]pyrimidin-5-yl]ethoxy}ethanol

Using 2-[2-(4-chloro-5H-pyrrolo[3,2-d]pyrimidin-5-yl)ethoxy]ethyl benzoate (444 mg), 4-[3-(4-tert-butyl-1,3-oxazol-2-yl)phenoxy]-3-chloroaniline (660 mg) and isopropyl alcohol (10 mL), the reaction was carried out in the same manner as in Example C-2(v). Then, the obtained compound was reacted in the same manner as in Example C-2(v) and using 1N aqueous sodium hydroxide solution (6.0 mL) and tetrahydrofuran (12 mL) to give the title compound (316 mg) as a white powder.

¹H-NMR (CDCl₃) δ: 1.30 (9H, s), 3.70-3.80 (4H, m), 4.02 (2H, t, J=4.2 Hz), 4.56 (2H, t, J=4.2 Hz), 6.62 (1H, d, J=3.3 Hz), 7.00 (1H, dd, J=2.4 Hz, 8.4 Hz), 7.05 (1H, d, J=8.7 Hz), 7.20 (1H, d, J=3..3 Hz), 7.34 (1H, s), 7.37 (1H, t, J=7.8 Hz), 7.59 (1H, dd, J=2.4 Hz, 9.0 Hz), 7.60-7.65 (1H, m), 7.75 (1H, d, J=7.8 Hz), 7.89 (1H, d, J=2.7 Hz), 8.51 (1H, s), 8.77 (1H, s).

### Example C-6

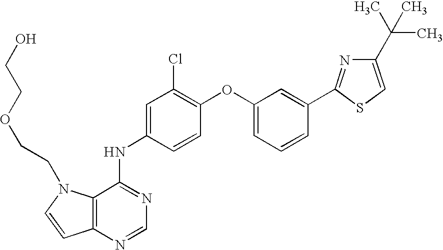

### Production of 2-{2-[4-({4-[3-(4-tert-butyl-1,3-thiazol-2-yl)phenoxy]-3-chlorophenyl}amino)-5H-pyrrolo[3,2-d]pyrimidin-5-yl]ethoxy}ethanol

### (i) Production of 3-methoxybenzenecarbothioamide

A mixture of 3-methoxybenzonitrile (9.32 g), O,O-diethyl dithiophosphate (11.85 mL) and 4N hydrochloric acid (70 mL) was stirred at room temperature for 20 hr. The precipitate was collected by filtration, and washed with ethyl acetate and diisopropyl ether to give the title compound (8.51 g) as a pale-green powder.

¹H-NMR (CDCl₃) δ: 3.27 (2H, br s), 3.89 (3H, s), 7.10-7.20 (1H, m), 7.36 (1H, t, J=7.8 Hz), 7.40-7.50 (1H, m), 7.50-7.60 (1H, m).

### (ii) Production of 4-tert-butyl-2-(3-methoxyphenyl)-1,3-thiazole

A solution of 3-methoxybenzenecarbothioamide (4.18 g) and 1-bromopinacolone (4.48 g) in ethanol (50 mL) was stirred at room temperature for 1 hr. Water was added to the reaction mixture and the mixture was extracted with ethyl acetate. The organic layer was washed successively with aqueous sodium bicarbonate and brine, and dried over anhydrous magnesium sulfate. After concentration under reduced pressure, the residue was separated and purified by silica gel column chromatography (eluent, ethyl acetate:hexane=0:100→10:90) to give the title compound (4.91 g) as a colorless oil.

¹H-NMR (CDCl₃) δ: 1.39 (9H, s), 3.88 (3H, s), 6.88 (1H, s), 6.90-7.00 (1H, m), 7.32 (1H, t, J=8.1 Hz), 7.50-7.60 (2H, m).

### (iii) Production of 3-(4-tert-butyl-1,3-thiazol-2-yl)phenol

Using 4-tert-butyl-2-(3-methoxyphenyl)-1,3-thiazole (4.91 g) and 48% hydrobromic acid (30 mL) and in the same manner as in Example C-4(ii), the title compound (3.59 g) was obtained as a colorless oil.

¹H-NMR (CDCl₃) δ: 1.40 (9H, s), 5.08 (1H, s), 6.80-6.85 (1H, m), 6.89 (1H, s), 7.28 (1H, t, J=8.0 Hz), 7.45-7.55 (2H, m).

### (iv) Production of 4-tert-butyl-2-[3-(2-chloro-4-nitrophenoxy)phenyl]-1,3-thiazole

Using 3-(4-tert-butyl-1,3-thiazol-2-yl)phenol (3.13 g), 3-chloro-4-fluoronitrobenzene (2.48 g), potassium carbonate (2.78 g) and N,N-dimethylformamide (24 mL) and in the same manner as in Example C-1(i), the title compound (1.49 g) was obtained as a pale-yellow oil.

¹H-NMR (CDCl₃) δ: 1.38 (9H, s), 6.93 (2H, t, J=4.6 Hz), 7.05-7.15 (1H, m), 7.49 (1H, t, J=8.0 Hz), 7.75-7.80 (1H, m), 7.80-7.90 (1H, m), 8.06 (1H, dd, J=2.6, 8.8 Hz), 8.40 (1H, d, J=2.6 Hz).

### (v) Production of 4-[3-(4-tert-butyl-1,3-thiazol-2-yl)phenoxy]-3-chloroaniline

Using 4-tert-butyl-2-[3-(2-chloro-4-nitrophenoxy)phenyl]-1,3-thiazole (1.49 g), 5% platinum-activated carbon (0.25 g) and ethyl acetate (10 mL) and in the same manner as in Example C-1(ii), the title compound (1.37 g) was obtained as a pale-yellow oil.

¹H-NMR (CDCl₃) δ: 1.38 (9H, s), 3.68 (2H, br s), 6.58 (1H, dd, J=2.8, 8.6 Hz), 6.80-6.95 (4H, m), 7.30 (1H, t, J=8.1 Hz), 7.55-7.65 (2H, m).

### (vi) Production of 2-{2-[4-({4-[3-(4-tert-butyl-1,3-thiazol-2-yl)phenoxy]-3-chlorophenyl}amino)-5H-pyrrolo[3,2-d]pyrimidin-5-yl]ethoxy}ethanol

Using 2-[2-(4-chloro-5H-pyrrolo[3,2-d]pyrimidin-5-yl)ethoxy]ethyl benzoate (277 mg), 4-[3-(4-tert-butyl-1,3-thiazol-2-yl)phenoxy]-3-chloroaniline (359 mg) and isopropyl alcohol (5.0 mL), the reaction was carried out lo in the same manner as in Example C-2(v). Then, the obtained compound was reacted in the same manner as in Example C-2(v) and using 1N aqueous sodium hydroxide solution (3.7 mL) and tetrahydrofuran (7.5 mL) to give the title compound (163 mg) as a white powder.

¹H-NMR (CDCl₃) δ: 1.39 (9H, s), 3.70-3.80 (4H, m), 4.02 (2H, t, J=4.5 Hz), 4.57 (2H, t, J=4.5 Hz), 6.63 (1H, d, J=3.3 Hz), 6.89 (1H, s), 6.92 (1H, d, J=2.4 Hz), 7.05 (1H, d, J=9.0 Hz), 7.21 (1H, d, J=3.0 Hz), 7.34 (1H, t, J=8.4 Hz), 7.58 (1H, dd, J=2.4 Hz, 8.7 Hz), 7.65-7.70 (2H, m), 7.89 (1H, d, J=2.4 Hz), 8.52 (1H, s), 8.75 (1H, s).

### Example C-7

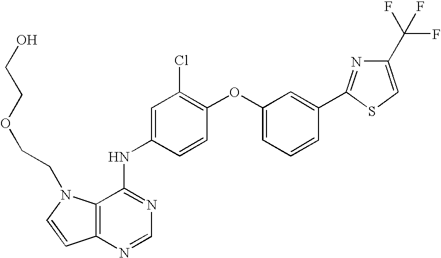

### Production of 2-(2-{4-[(3-chloro-4-{3-[4-(trifluoromethyl)-1,3-thiazol-2-yl]phenoxy}phenyl)amino]-5H-pyrrolo[3,2-d]pyrimidin-5-yl}ethoxy)ethanol

### (i) Production of 2-(3-methoxyphenyl)-4-(trifluoromethyl)-1,3-thiazole

Using 3-methoxybenzenecarbothioamide (4.18 g), 3-bromo-1,1,1-trifluoroacetone (4.77 g) and ethanol (50 mL) and in the same manner as in Example C-6(ii), the title compound (4.29 g) was obtained as a pale-yellow oil.

¹H-NMR (CDCl₃) δ: 3.90 (3H, s), 6.95-7.05 (1H, m), 7.37 (1H, d, J=8.2 Hz), 7.50-7.60 (2H, m), 7.73 (1H, d, J=1.0 Hz).

### (ii) Production of 3-[4-(trifluoromethyl)-1,3-thiazol-2-yl]phenol

Using 2-(3-methoxyphenyl)-4-(trifluoromethyl)-1,3-thiazole (4.23 g) and 48% hydrobromic acid (30 mL) and in the same manner as in Example C-4(ii), the title compound (4.61 g) was obtained as a yellow oil.

¹H-NMR (CDCl₃) δ: 5.10-5.50 (1H, m), 6.90-7.00 (1H, m), 7.33 (1H, t, J=8.1 Hz), 7.50-7.60 (2H, m), 7.73 (1H, s).

### (iii) Production of 2-[3-(2-chloro-4-nitrophenoxy)phenyl]-4-(trifluoromethyl)-1,3-thiazole

Using 3-[4-(trifluoromethyl)-1,3-thiazol-2-yl]phenol (4.00 g), 3-chloro-4-fluoronitrobenzene (3.01 g), potassium carbonate (3.38 g) and N,N-dimethylformamide (20 mL) and in the same manner as in Example C-1(i), the title compound (4.82 g) was obtained as a pale-yellow powder.

¹H-NMR (CDCl₃) δ: 6.96 (1H, t, J=9.0 Hz), 7.15-7.25 (1H, m), 7.55 (1H, t, J=8.1 Hz), 7.75-7.80 (2H, m), 7.85 (1H, t, J=8.1 Hz), 8.09 (1H, dd, J=2.7 Hz, 9.0 Hz), 8.41 (1H, d, J=2.7 Hz).

### (iv) Production of 3-chloro-4-{3-[4-(trifluoromethyl)-1,3-thiazol-2-yl]phenoxy}aniline

Using2-[3-(2-chloro-4-nitrophenoxy)phenyl]-4-(trifluoromethyl)-1,3-thiazole (2.00 g), 5% platinum-activated carbon (0.33 g) and ethyl acetate (15 mL) and in the same manner as in Example C-1(ii), the title compound (1.87 g) was obtained as a colorless oil.

¹H-NMR (CDCl₃) δ: 3.70 (2H, br s), 6.59 (1H, dd, J=2.7 Hz, 8.7 Hz), 6.81 (1H, d, J=2.7 Hz), 6.94 (2H, d, J=8.7 Hz), 7.36 (1H, t, J=8.1 Hz), 7.50-7.55 (1H, m), 7.63 (1H, d, J=7.5 Hz), 7.73 (1H, s).

### (v) Production of 2-(2-{4-[(3-chloro-4-{3-[4-(trifluoromethyl)-1,3-thiazol-2-yl]phenoxy}phenyl)amino]-5H-pyrrolo[3,2-d]pyrimidin-5-yl}ethoxy)ethanol

Using 2-[2-(4-chloro-5H-pyrrolo[3,2-d]pyrimidin-5-yl)ethoxy]ethyl benzoate (262 mg), 3-chloro-4-{3-[4-(trifluoromethyl)-1,3-thiazol-2-yl]phenoxy}aniline (338 mg) and isopropyl alcohol (5.0 mL), the reaction was carried out in the same manner as in Example C-2(v). Then, the obtained compound was reacted in the same manner as in Example C-2(v) and using 1N aqueous sodium hydroxide solution (3.4 mL) and tetrahydrofuran (7 mL) to give the title compound (173 mg) as a white powder.

¹H-NMR (CDCl₃) δ: 3.70-3.85 (4H, m), 4.02 (2H, t, J=4.5 Hz), 4.57 (2H, t, J=4.5 Hz), 6.63 (1H, d, J=3.3 Hz), 7.00-7.10 (1H, m), 7.08 (1H, d, J=8.7 Hz), 7.21 (1H, d, J=3.0 Hz), 7.39 (1H, t, J=8.0 Hz), 7.6-7.65 (2H, 25 m), 7.67 (1H, d, J=7.8 Hz), 7.74 (1H, s), 7.91 (1H, d, J=2.7 Hz), 8.52 (1H, s), 8.79 (1H, s).

### Example C-8

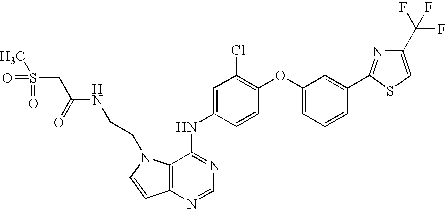

### Production of N-(2-{4-[(3-chloro-4-{3-[4-(trifluoromethyl)-1,3-thiazol-2-yl]phenoxy}phenyl)amino]-5H-pyrrolo[3,2-d]pyrimidin-5-yl}ethyl)-2-(methylsulfonyl)acetamide

### (i) Production of tert-butyl(2-{4-[(3-chloro-4-{3-[4-(trifluoromethyl)-1,3-thiazol-2-yl]phenoxy}phenyl)amino]-5H-pyrrolo[3,2-d]pyrimidin-5-yl}ethyl)carbamate

A mixture of tert-butyl[2-(4-chloro-5H-pyrrolo[3,2-d]pyrimidin-5-yl)ethyl]carbamate (1.01 g), 3-chloro-4-{3-[4-(trifluoromethyl)-1,3-thiazol-2-yl]phenoxy}aniline (1.51 g) and isopropyl alcohol (10 mL) was stirred at 80° C. for 12 hr. To the reaction mixture was added aqueous sodium bicarbonate under ice-cooling, and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:hexane=60:40→100:0). The obtained solid was collected by filtration, washed with diisopropyl ether and hexane to give the title compound (1.88 g) as a white powder.

¹H-NMR (CDCl₃) δ: 1.50 (9H, s), 3.45-3.55 (2H, m), 4.45-4.55 (2H, m), 5.05-5.15 (1H, m), 6.61 (1H, d, J=1.5 Hz), 7.00-7.10 (2H, m), 7.18 (1H, d, J=2.1 Hz), 7.40 (1H, t, J=8.5 Hz), 7.65 (1H, s), 7.68 (1H, d, J=7.5 Hz), 7.74 (1H, s), 7.89 (1H, d, J=9.0 Hz), 8.03 (1H, s), 8.51 (1H, s), 8.61 (1H, br s).

### (ii) Production of 5-(2-aminoethyl)-N-(3-chloro-4-{3-[4-(trifluoromethyl)-1,3-thiazol-2-yl]phenoxy}phenyl)-5H-pyrrolo[3,2-d]pyrimidin-4-amine dihydrochloride

A mixture of tert-butyl(2-{4-[(3-chloro-4-{3-[4(trifluoromethyl)-1,3-thiazol-2-yl]phenoxy}phenyl)amino]-5H-pyrrolo[3,2-d]pyrimidin-5-yl}ethyl)carbamate (1.70 g) and 10% (W/W) hydrochloric acid/methanol (12 mL) was stirred at 65° C. for 4 hr. The reaction mixture was concentrated under reduced pressure, and the precipitate was collected by filtration, and washed with diethyl ether to give the title compound (1.53 g) as pale-yellow crystals.

¹H-NMR (DMSO-d₆) δ: 3.25-3.35 (2H, m), 5.00-5.1.0 (2H, m), 6.75 (1H, d, J=3.3 Hz), 7.17 (1H, dd, J=2.4, 8.1 Hz), 7.35 (1H, d, J=8.7 Hz), 7.5-7.7 (3H, m), 7.78 (1H, d, J=7.8 Hz), 7.93 (1H, d, J=2.4 Hz), 8.07 (1H, d, J=3.0 Hz), 8.20-8.40 (3H, m), 8.61 (1H, s), 8.72 (1H, s), 10.10 (1H, br s).

### (iii) Production of N-(2-{4-[(3-chloro-4-{3-[4-(trifluoromethyl)-1,3-thiazol-2-yl]phenoxy}phenyl)amino]-5H-pyrrolo[3,2-d]pyrimidin-5-yl}ethyl)-2-(methylsulfonyl)acetamide

To a solution of 5-(2-aminoethyl)-N-(3-chloro-4-{3-[4-(trifluoromethyl)-1,3-thiazol-2-yl]phenoxy}phenyl)-5H-pyrrolo[3,2-d]pyrimidin-4-amine dihydrochloride (200 mg), methylsulfonylacetic acid (69 mg) and 1-hydroxybenzotriazole (75 mg) in N,N-dimethylformamide (5.0 mL) were added triethylamine (0.23 mL) and 1-ethyl-3-(3-dimethylaminopropyl)carbodiimide hydrochloride (105 mg) under ice-cooling, and the mixture was stirred at room temperature for 6 hr. Water was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→95:5) and further recrystallized from ethyl acetate/diisopropyl ether to give the title compound (179 mg) as colorless crystals.

¹H-NMR (CDCl₃) δ: 3.12 (3H, s), 3.65-3.75 (2H, m), 3.98 (2H, s), 4.45-4.55 (2H, m), 6.60-6,65 (1H, m), 7.08 (2H, d, J=9.0 Hz), 7.21 (1H, d, J=3.0 Hz), 7.25-7.30 (2H, m), 7.42 (1H, t, J=8.0 Hz), 7.65-7.75 (2H, m), 7.75 (1H, s), 7.95 (1H, s), 8.20 (1H, s), 8.51 (1H, s).

### Example C-9

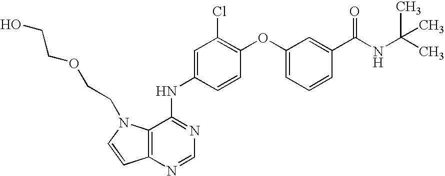

### Production of N-(tert-butyl)-3-[2-chloro-4-({5-[2-(2-hydroxyethoxy)ethyl]-5H-pyrrolo[3,2-d]pyrimidin-4-yl}amino)phenoxy]benzamide

### (i) Production of diphenylmethyl 3-hydroxybenzoate

To a solution of 3-hydroxybenzoic acid (2.76 g) in acetone (40 mL) was added diphenyldiazomethane (3.88 g) under ice-cooling, and the mixture was stirred at room temperature overnight. The reaction mixture was concentrated under reduced pressure and the obtained residue was subjected to silica gel column chromatography (eluent, ethyl acetate:hexane=15:85→35:65). The objective fractions were concentrated under reduced pressure to give the title compound (5.16 g) as a pale-yellow oil.

¹H-NMR (CDCl₃) δ: 5.13 (1H, s), 7.03-7.08 (1H, m), 7.10 (1H, s), 7.25-7.46 (11H, m), 7.58-7.62 (1H, m), 7.70-7.76 (1H, m).

### (ii) Production of diphenylmethyl 3-(2-chloro-4-nitrophenoxy)benzoate

A mixture of 2-chloro-1-fluoro-4-nitrobenzene (2.81 g), diphenylmethyl 3-hydroxybenzoate (5.16 g), potassium carbonate (3.32 g) and N,N-dimethylformamide (50 mL) was lo stirred at room temperature overnight. The reaction mixture was concentrated under reduced pressure, water was added and the mixture was extracted with ethyl acetate. The ethyl acetate layer was washed with saturated brine and dried over anhydrous magnesium sulfate. The solvent was evaporated under reduced pressure, diisopropyl ether was added to the obtained residue, and the precipitated solid was collected by filtration to give the title compound (6.93 g) as a colorless powder.

¹H-NMR (CDCl₃) δ: 6.90 (1H, d, J=9.3 Hz), 7.12 (1H, s), 7.27-7.46 (11H, m), 7.55 (1H, t, J=8.0 Hz), 7.82 (1H, m), 8.02-8.11 (2H, m), 8.40 (1H, d, J=2.7 Hz).

### (iii) Production of diphenylmethyl 3-(4-amino-2-chlorophenoxy)benzoate

To diphenylmethyl 3-(2-chloro-4-nitrophenoxy)benzoate (4.60 g) were added ethyl acetate (80 mL) and 5% platinum-activated carbon (50 mg) and the mixture was stirred under a hydrogen atmosphere at room temperature for 5 hr. The catalyst was filtered off, the filtrate was concentrated and the obtained residue was subjected to basic silica gel column chromatography (eluent, ethyl acetate:hexane=20:80→40:60), and silica gel column chromatography (eluent, ethyl acetate:hexane=15:85→35:65). The objective fractions were concentrated under reduced pressure to give the title compound (3.58 g) as a colorless solid.

¹H-NMR (CDCl₃) δ: 3.69 (2H, br s), 6.57 (1H, dd, J=2.7 Hz, 8.6 Hz), 6.79 (1H, d, J=2.7 Hz), 6.91 (1H, d, J=8.6 Hz), 7.04-7.10 (2H, m), 7.26-7.44 (11H, m), 7.62-7.65 (1H, m), 7.78-7.83 (1H, m).

### (iv) Production of 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride

A mixture of 2-[2-(4-chloro-5H-pyrrolo[3,2-d]pyrimidin-5-yl)ethoxy]ethyl benzoate (1.04 g), diphenylmethyl 3-(4-amino-2-chlorophenoxy)benzoate (1.29 g) and isopropyl alcohol (20 mL) was stirred at 80° C. overnight. An aqueous sodium hydrogencarbonate solution was added to the reaction mixture and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure. To the residue were added trifluoroacetic acid (10 mL) and anisole (10 mL) and the mixture was stirred at room temperature for 4 hr. The reaction mixture was concentrated under reduced pressure. 4N Hydrogen chloride/ethyl acetate solution was added to the residue, and the mixture was concentrated under reduced pressure. Ethyl acetate and acetonitrile were added to the residue and the precipitated solid was collected by filtration to give the title compound (1.24 g) as a white powder.

¹H-NMR (DMSO-d₆) δ: 3.76-3.83 (2H, m), 3.91 (2H, t, J=4.7 Hz), 4.27-4.33 (2H, m), 4.89 (2H, m), 6.60-6.64 (1H, m), 7.22 (1H, d, J=8.8 Hz), 7.26-7.75 (10H, m), 7.91 (1H, d, J=2.5 Hz), 8.01 (1H, d, J=3.0 Hz), 8.64 (1H, s), 9.91 (1H, m).

### (v) Production of N-(tert-butyl)-3-[2-chloro-4-({5-[2-(2-hydroxyethoxy)ethyl]-5H-pyrrolo[3,2-d]pyrimidin-4-yl}amino)phenoxy]benzamide

A mixture of 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), tert-butylamine (0.038 mL), 1-ethyl-3-(3-dimethylaminopropyl)carbodiimide hydrochloride (69 mg), 1-hydroxybenzotriazole (55 mg), triethylamine (0.050 mL) and N,N-dimethylformamide (3 mL) was stirred at room temperature overnight. Water was added to the reaction mixture, and the mixture was extracted with ethyl acetate. The ethyl acetate layer was washed with saturated brine and dried over anhydrous magnesium sulfate. The solvent was evaporated under reduced pressure and the obtained residue was subjected to silica gel column chromatography (eluent, methanol:ethyl acetate=0:100→10:90). The objective fractions were concentrated under reduced pressure. To the residue were added methanol (5 mL), tetrahydrofuran (1 mL) and 1N aqueous sodium hydroxide solution (0.6 mL) and the mixture was stirred at room temperature overnight. Water was added to the reaction mixture, and the mixture was extracted with ethyl acetate. The ethyl acetate layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate-diethyl ether to give the title compound (106 mg) as white crystals.

¹H-NMR (CDCl₃) δ: 1.45 (9H, s), 2.36 (1H, br s), 3.69-3.81 (4H, m), 3.99-4.05 (2H, m), 4.53-4.60 (2H, m), 5.96 (1H, br s), 6.61 (1H, d, J=3.0 Hz), 7.03 (1H, d, J=8.8 Hz), 7.05-7.12 (1H, m), 7.21 (1H, d, J=3.0 Hz), 7.27-7.37 (3H, m), 7.57 (1H, dd, J=2.5 Hz, 8.8 Hz), 7.91 (1H, d, J=2.5 Hz), 8.51 (1H, s), 8.79 (1H, br s).

### Example C-10

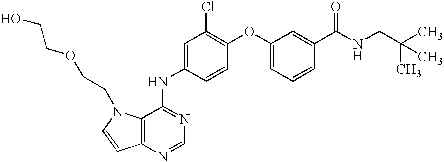

### Production of 3-[2-chloro-4-({5-[2-(2-hydroxyethoxy)ethyl]-5H-pyrrolo[3,2-d]pyrimidin-4-yl}amino)phenoxy]-N-(2,2-dimethylpropyl)benzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), neopentylamine (0.042 mL), 1-ethyl-3-(3-dimethylaminopropyl)carbodiimide hydrochloride (69 mg), 1-hydroxybenzotriazole (55 mg), triethylamine (0.050 mL), N,N-dimethylformamide (3 mL), methanol (5 mL), tetrahydrofuran (1 mL) and 1N aqueous sodium hydroxide solution (0.6 mL) and in the same manner as in Example C-9(v), the title compound (116 mg) was obtained as white crystals.

¹H-NMR (CDCl₃) δ: 0.97 (9H, s), 2.30 (1H, br s), 3.25 (2H, d, J=6.3 Hz), 3.69-3.81 (4H, m), 3.99-4.05 (2H, m), 4.53-4.60 (2H, m), 6.14-6.26 (1H, m), 6.61 (1H, d, J=3.3 Hz), 7.04 (1H, d, J=8.8 Hz), 7.06-7.12 (1H, m), 7.21 (1H, d, J=3.3 Hz), 7.32-7.44 (3H, m), 7.57 (1H, dd, J=2.5 Hz, 8.8 Hz), 7.90 (1H, d, J=2.5 Hz), 8.51 (1H, s), 8.79 (1H, br s).

### Example C-11

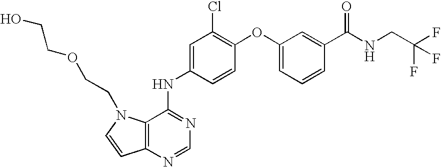

### Production of 3-[2-chloro-4-({5-[2-(2-hydroxyethoxy)ethyl]-5H-pyrrolo[3,2-d]pyrimidin-4-yl}amino)phenoxy]-N-(2,2,2-trifluoroethyl)benzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), 2,2,2-trifluoroethylamine (0.029 mL), 1-ethyl-3-(3-dimethylaminopropyl)carbodiimide hydrochloride (69 mg), 1-hydroxybenzotriazole (55 mg), triethylamine (0.050 mL), N,N-dimethylformamide (3 mL), methanol (5 mL), tetrahydrofuran (1 mL) and 1N aqueous sodium hydroxide solution (0.6 mL) and in the same manner as in Example C-9(v), the title compound (125 mg) was obtained as white crystals.

¹H-NMR (CDCl₃) δ: 2.11 (1H, br s), 3.70-3.82 (4H, m), 3.99-4.17 (4H, m), 4.54-4.62 (2H, m), 6.61 (1H, d, J=3.0 Hz), 6.67-6.78 (1H, m), 7.03 (1H, d, J=8.8 Hz), 7.14-7.20 (1H, m), 7.21 (1H, d, J=3.0 Hz), 7.33 (1H, m), 7.40 (1H, t, J=8.0 Hz), 7.46-7.51 (1H, m), 7.55 (1H, dd, J=8.8, 2.6 Hz), 7.88 (1H, d, J=2.6 Hz), 8.46 (1H, s), 8.78 (1H, br s).

### Example C-12

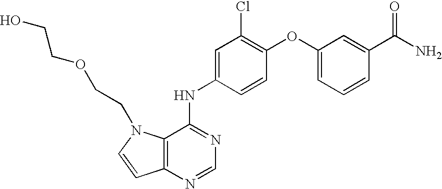

### Production of 3-[2-chloro-4-({5-[2-(2-hydroxyethoxy)ethyl]-5H-pyrrolo[3,2-d]pyrimidin-4-yl}amino)phenoxy]benzamide

To a solution of 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg) in N,N-dimethylformamide (3 mL) were added triethylamine (0.050 mL) and 1,1′-carbonylbis(1H-imidazole) (58 mg) and the mixture was stirred at room temperature for 0.5 hr. 7N ammonia/methanol (0.086 mL) was added and the mixture was stirred at room temperature for 4 hr. Water was added to the reaction mixture, and the mixture was extracted with ethyl acetate. The organic layer was washed with saturated brine and dried over anhydrous magnesium sulfate. The, solvent was evaporated under reduced pressure and the obtained residue was subjected to silica gel column chromatography (eluent, methanol:ethyl acetate=0:100→15:85). The objective fractions were concentrated under reduced pressure. To the residue were added methanol (5 mL), tetrahydrofuran (1 mL) and 1N aqueous sodium hydroxide solution (0.6 mL) and the mixture was stirred at room temperature overnight. Water was added to the reaction mixture, and the mixture was extracted with ethyl acetate. The ethyl acetate layer was washed with saturated brine and dried over anhydrous magnesium sulfate. The solvent was evaporated under reduced pressure and the obtained residue was subjected to basic silica gel column chromatography (eluent, methanol:ethyl acetate=0:100→15:85). The objective fractions were concentrated under reduced pressure. The residue was crystallized from ethanol-ethyl acetate to give the title compound (95 mg) as white crystals.

¹H-NMR (DMSO-d₆) δ: 3.49 (4H, m), 3.84 (2H, t, J=4.4 Hz), 4.65 (2H, t, J=4.4 Hz), 4.72 (1H, t, J=4.4 Hz), 6.52 (1H, d, J=2.7 Hz), 7.08-7.15 (1H, m), 7.21 (1H, d, J=8.7 Hz), 7.36-7.50 (3H, m), 7.58-7.72 (3H, m), 7.98-8.08 (2H, m), 8.35 (1H, s), 8.97 (1H, br s).

### Example C-13

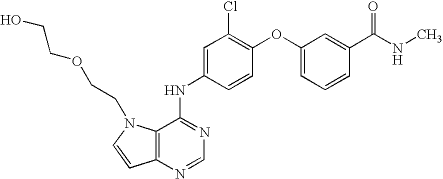

### Production of 3-[2-chloro-4-({5-[2-(2-hydroxyethoxy)ethyl]-5H-pyrrolo[3,2-d]pyrimidin-4-yl}amino)phenoxy]-N-methylbenzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), N,N-dimethylformamide (3 mL), triethylamine (0.050 mL), 1,1′-carbonylbis(1H-imidazole) (58 mg), 2M methylamine/tetrahydrofuran (0.30 mL), methanol (5 mL), tetrahydrofuran (1 mL) and 1N aqueous sodium hydroxide solution (0.6 mL) and in the same manner as in Example C-12, the title compound (114 mg) was obtained as white crystals.

¹H-NMR (DMSO-d₆) δ: 2.76 (3H, d, J=4.5 Hz), 3.50 (4H, m), 3.84 (2H, t, J=4.4 Hz), 4.65 (2H, t, J=4.4 Hz), 4.72 (1H, t, J=4.5 Hz), 6.52 (1H, d, J=3.0 Hz), 7.07-7.15 (1H, m), 7.20 (1H, d, J=8.7 Hz), 7.34 (1H, m), 7.46 (1H, t, J=7.8 Hz), 7.52-7.60 (1H, m), 7.61-7.73 (2H, m), 8.01 (1H, d, J=2.7 Hz), 8.35 (1H, s), 8.44-8.53 (1H, m), 8.97 (1H, br s).

### Example C-14

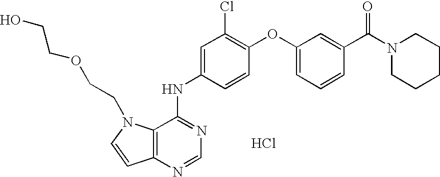

### Production of 2-{2-[4-({3-chloro-4-[3-(piperidin-1-ylcarbonyl)phenoxy]phenyl}amino)-5H-pyrrolo[3,2-d]pyrimidin-5-yl]ethoxy}ethanol hydrochloride

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), N,N-dimethylformamide (3 mL), triethylamine (0.050 mL), 1,1′-carbonylbis(1H-imidazole) (58 mg), piperidine (0.059 mL), methanol (5 mL), tetrahydrofuran (1 mL) and 1N aqueous sodium hydroxide solution (0.6 mL) and in the same manner as in Example C-12, 2-{2-[4-({3-chloro-4-[3-(piperidin-1-ylcarbonyl)phenoxy]phenyl}amino)-5H-pyrrolo[3,2-d]pyrimidin-5-yl]ethoxy}ethanol was obtained. The compound was dissolved in ethyl acetate-ethanol and 1N hydrogen chloride/ethyl acetate solution (0.3 mL) was added. The solvent was evaporated under reduced pressure and the obtained residue was crystallized from ethanol-ethyl acetate to give the title compound (126 mg) as white crystals.

¹H-NMR (DMSO-d₆) δ: 1.34-1.68 (6H, m), 3.15-3.75 (8H, m), 3.84 (2H, t, J=4.5 Hz), 4.81 (2H, m), 6.70 (1H, d, J=3.0 Hz), 6.86 (1H, m), 7.04-7.10 (1H, m), 7.12 (1H, d, J=7.7 Hz), 7.31 (1H, d, J=8.8 Hz), 7.44-7.51 (1H, m), 7.64 (1H, dd, J=2.5 Hz, 8.8 Hz), 7.97 (1H, d, J=2.5 Hz), 8.02 (1H, d, J=3.3 Hz), 8.74 (1H, s), 9.90 (1H, br s).

### Example C-15

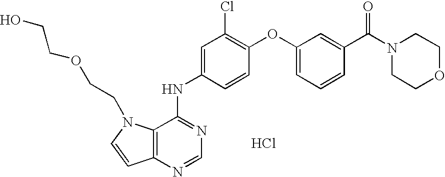

### Production of 2-{2-[4-({3-chloro-4-[3-(morpholin-4-ylcarbonyl)phenoxy]phenyl}amino)-5H-pyrrolo[3,2-d]pyrimidin-5-yl]ethoxy}ethanol hydrochloride

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), morpholine (0.031 mL), 1-ethyl-3-(3-dimethylaminapropyl)carbodiimide hydrochloride (69 mg), 1-hydroxybenzotriazole (55 mg), triethylamine (0.050 mL), N,N-dimethylformamide (3 mL), methanol (5 mL), tetrahydrofuran (1 mL) and 1N aqueous sodium hydroxide solution (0.6 mL) and in the same manner as in Example. C-9(v), 2-{2-[4-({3-chloro-4-[3-(morpholin-4-ylcarbonyl)phenoxy]phenyl}amino)-5H-pyrrolo[3,2-d]pyrimidin-5-yl]ethoxy}ethanol was obtained. The compound was dissolved in ethyl acetate-ethanol, and 1N hydrogen chloride/ethyl acetate solution (0.3 mL) was added. The solvent was evaporated under reduced pressure and the obtained residue was crystallized from ethanol-ethyl acetate to give the title compound (116 mg) as white crystals.

¹H-NMR (DMSO-d₆) δ: 3.20-3.80 (12H, m), 3.85 (2H, t, J=4.4 Hz), 4.81 (2H, t, J=4.4 Hz), 6.70 (1H, d, J=3.0 Hz), 6.94 (1H, m), 7.05-7.12 (1H, m), 7.15-7.21 (1H, m), 7.30 (1H, d, J=8.8 Hz), 7.45-7.53 (1H, m), 7.64 (1H, dd, J=2.5 Hz, 8.8 Hz), 7.97 (1H, d, J=2.5 Hz), 8.02 (1H, d, J=3.3 Hz), 8.74 (1H, s), 9.90 (1H, br s).

### Example C-16

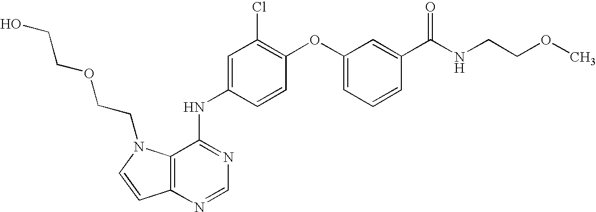

### Production of 3-[2-chloro-4-({5-[2-(2-hydroxyethoxy)ethyl]-5H-pyrrolo[3,2-d]pyrimidin-4-yl}amino)phenoxy]-N-(2-methoxyethyl)benzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), 2-methoxyethylamine (0.031 mL), 1-ethyl-3-(3-dimethylaminopropyl)carbodiimide hydrochloride (69 mg), 1-hydroxybenzotriazole (55 mg), triethylamine (0.050 mL), N,N-dimethylformamide (3 mL), methanol (5 mL), tetrahydrofuran (1 mL) and 1N aqueous sodium hydroxide solution (0.6 mL) and in the same manner as in Example C-9(v), the title compound (134 mg) was obtained as white crystals.

¹H-NMR (CDCl₃) δ: 2.07-2.31 (1H, m), 3.38 (3H, s), 3.51-3.66 (4H, m), 3.69-3.81 (4H, m), 3.99-4.05 (2H, m), 4.54-4.60 (2H, m), 6.51-6.59 (1H, m), 6.62 (1H, d, J=3.3 Hz), 7.04 (1H, d, J=8.8 Hz), 7.08-7.13 (1H, m), 7.21 (1H, d, J=3.3 Hz), 7.31-7.46 (3H, m), 7.58 (1H, dd, J=8.8, 2.8 Hz), 7.90 (1H, d, J=2.8 Hz), 8.51 (1H, s), 8.78 (1H, br s).

### Example C-17

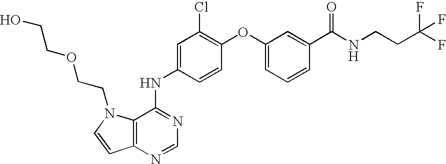

### Production of 3-[2-chloro-4-({5-[2-(2-hydroxyethoxy)ethyl]-5H-pyrrolo[3,2-d]pyrimidin-4-yl}amino)phenoxy]-N-(3,3,3-trifluoropropyl)benzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), 3,3,3-trifluoropropylamine hydrochloride (53 mg), 1-ethyl-3-(3-dimethylaminopropyl)carbodiimide hydrochloride (69 mg), 1-hydroxybenzotriazole (55 mg), triethylamine (0.092 mL), N,N-dimethylformamide (3 mL), methanol (5 mL), tetrahydrofuran (1 mL) and 1N aqueous sodium hydroxide solution (0.6 mL) and in the same manner as in Example C-9(v), the title compound (150 mg) was obtained as white crystals.

¹H-NMR (CDCl₃) δ: 2.08 (1H, br s), 2.37-2.54 (2H, m), 3.64-3.83 (6H, m), 3.99-4.06 (2H, m), 4.55-4.61 (2H, m), 6.48-6.58 (1H, m), 6.62 (1H, d, J=3.2 Hz), 7.04 (1H, d, J=9.0 Hz), 7.11-7.17 (1H, m), 7.22 (1H, d, J=3.2 Hz), 7.27 (1H, m), 7.34-7.45 (2H, m), 7.57 (1H, dd, J=2.5 Hz, 9.0 Hz), 7.89 (1H, d, J=2.5 Hz), 8.50 (1H, s), 8.78 (1H, br s).

### Example C-18

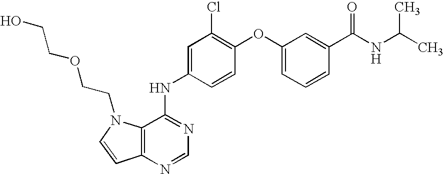

### Production of 3-[2-chloro-4-({5-[2-(2-hydroxyethoxy)ethyl]-5H-pyrrolo[3,2-d]pyrimidin-4-yl}amino)phenoxy]-N-isopropylbenzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), isopropylamine (0.031 mL), 1-ethyl-3-(3-dimethylaminopropyl)carbodiimide hydrochloride (69 mg), 1-hydroxybenzotriazole (55 mg), triethylamine (0.050 mL), N,N-dimethylformamide (3 mL), methanol (5 mL), tetrahydrofuran (1 mL) and 1N aqueous sodium hydroxide solution (0.6 mL) and in the same manner as in Example C-9(v), the title compound (125 mg) was obtained as white crystals.

¹H-NMR (CDCl₃) δ: 1.25 (6H, d, J=6.6 Hz), 2.13-2.37 (1H, m), 3.69-3.81 (4H, m), 3.99-4.05 (2H, m), 4.18-4.31 (1H, m), 4.53-4.60 (2H, m), 5.92-6.02 (1H, m), 6.62 (1H, d, J=3.0 Hz), 7.03 (1H, d, J=8.8 Hz), 7.06-7.12 (1H, m), 7.21 (1H, d, J=3.0 Hz), 7.30-7.42 (3H, m), 7.56 (1H, dd, J=2.8 Hz, 8.8 Hz), 7.90 (1H, d, J=2.8 Hz), 8.50 (1H, s), 8.78 (1H, br s).

### Example C-19

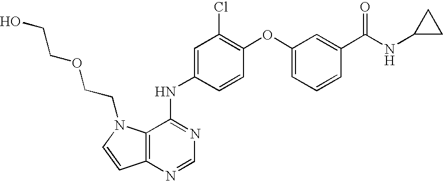

### Production of 3-[2-chloro-4-({5-[2-(2-hydroxyethoxy)ethyl]-5H-pyrrolo[3,2-d]pyrimidin-4-yl}amino)phenoxy]-N-cyclopropylbenzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), cyclopropylamine (0.025 mL), 1-ethyl-3-(3-dimethylaminopropyl)carbodiimide hydrochloride (69 mg), 1-hydroxybenzotriazole (55 mg), triethylamine (0.050 mL), N,N-dimethylformamide (3 mL), methanol (5 mL), tetrahydrofuran (1 mL) and 1N aqueous sodium hydroxide solution (0.6 mL) and in the same manner as, in Example C-9(v), the title compound (118 mg) was obtained as white crystals.

¹H-NMR (DMSO-d₆) δ: 0.52-0.73 (4H, m), 2.76-2.87 (1H, m), 3.49 (4H, m), 3.84 (2H, t, J=4.6 Hz), 4.65 (2H, t, J=4.6 Hz), 4.72 (1H, t, J=4.6 Hz), 6.52 (1H, d, J=3.2 Hz), 7.06-7.12 (1H, m), 7.19 (1H, d, J=8.9 Hz), 7.35 (1H, m), 7.44 (1H, t, J=7.8 Hz), 7.52-7.58 (1H, m), 7.64 (1H, dd, J=8.9, 2.5 Hz), 7.69 (1H, d, J=3.2 Hz), 8.00 (1H, d, J=2.5 Hz), 8.34 (1H, s), 8.49 (1H, d, J=4.1 Hz), 8.97 (1H, br s).

### Example C-20

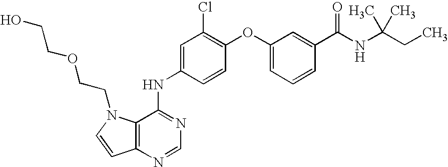

### Production of 3-[2-chloro-4-({5-[2-(2-hydroxyethoxy)ethyl]-5H-pyrrolo[3,2-d]pyrimidin-4-yl}amino)phenoxy]-N-(1,1-dimethylpropyl)benzamide

Using 3-{4-[(5-{2-[2-(benzoyloxy)ethoxy]ethyl}-5H-pyrrolo[3,2-d]pyrimidin-4-yl)amino]-2-chlorophenoxy}benzoic acid hydrochloride (183 mg), tert-amylamine (0.042 mL), 1-ethyl-3-(3-dimethylaminopropyl)carbodiimide hydrochloride (69 mg), 1-hydroxybenzotriazole (55 mg), triethylamine (0.050 mL), N,N-dimethylformamide (3 mL), methanol (5 mL), tetrahydrofuran (1 mL) and 1N aqueous sodium hydroxide solution (0.6 mL) and in the same manner as in Example C-9(v), the title compound (135 mg) was obtained as white crystals.

¹H-NMR (CDCl₃) δ: 0.90 (3H, t, J=7.5 Hz), 1.40 (6H, s), 1.83 (2H, q, J=7.5 Hz), 3.70-3.80 (4H, m), 3.99-4.05 (2H, m), 4.54-4.60 (2H, m), 5.84 (1H, br s), 6.63 (1H, d, J=3.2 Hz), 7.02-7.1.2 (2H, m), 7.21 (1H, d, J=3.2 Hz), 7.28-7.39 (3H, m), 7.58 (1H, dd, J=8.8, 2.7 Hz), 7.91 (1H, d, J=2.7 Hz), 8.51 (1H, s), 8.79 (1H, br s).

### Example C-21

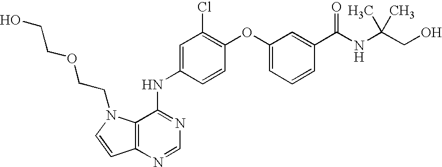

### Production of 3-[2-chloro-4-({5-[2-(2-hydroxyethoxy)ethyl]-5H-pyrrolo[3,2-d]pyrimidin-4-yl}amino)phenoxy]-N-(2-hydroxy-1,1-dimethylethyl)benzamide

Using 3-14-[(5-{2-[2-(benzoyloxy)ethoxy]ethyl}-5H-pyrrolo[3,2-d]pyrimidin-4-yl)amino]-2-chlorophenoxylbenzoic acid hydrochloride (183 mg), 2-amino-2-methyl-1-propanol (0.034 mL), 1-ethyl-3-(3-dimethylaminopropyl)carbodiimide hydrochloride (69 mg), 1-hydroxybenzotriazole (55 mg), triethylamine (0.050 mL), N,N-dimethylformamide (3 mL),-methanol (5 mL), tetrahydrofuran (1 mL) and 1N aqueous sodium hydroxide solution (0.6 mL) and in the same manner as in Example C-9(v), the title compound (106 mg) was obtained as white crystals.

¹H-NMR (CDCl₃) δ: 1.40 (6H, s), 3.67 (2H, s), 3.69-3.81 (4H, m), 3.98-4.05 (2H, m), 4.54-4.60 (2H, m), 6.23 (1H, br s), 6.62 (1H, d, J=3.0 Hz), 7.04 (1H, d, J=8.8 Hz), 7.08-7.15 (1H, m), 7.21 (1H, d, J=3.0 Hz), 7.28 (1H, m), 7.32-7.40 (2H, m), 7.57 (1H, dd, J=2.5 Hz, 8.8 Hz), 7.90 (1H, d, J=2.5 Hz), 8.49 (1H, s), 8.80 (1H, br s).

### Example C-22

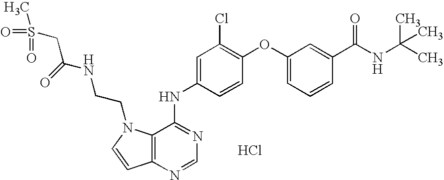

### Production of N-(tert-butyl)-3-(2-chloro-4-{[5-(2-{[methylsulfonyl)acetyl]amino}ethyl)-5H-pyrrolo[3,2-d]pyrimidin-4-yl]amino}phenoxy)benzamide hydrochloride

### (i) Production of methyl 3-{4-[(5-{2-[(tert-butoxycarbonyl)amino]ethyl}-5H-pyrrolo[3,2-d]pyrimidin-4-yl)amino]-2-chlorophenoxy}benzoate

A mixture of tert-butyl[2-(4-chloro-5H-pyrrolo[3,2-d]pyrimidin-5-yl)ethyl]carbamate (2.08 g), methyl 3-(4-amino-2-chlorophenoxy)benzoate (1.94 g) and isopropyl alcohol (20 mL) was stirred at 80° C. overnight. To the reaction mixture was added aqueous sodium hydrogencarbonate solution and the mixture was extracted with ethyl acetate. The ethyl acetate layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure. Ethyl acetate and diisopropyl ether were added to the residue, and the precipitated solid was collected by filtration to give the title compound (3.26 g) as a white powder.

¹H-NMR (CDCl₃) δ: 1.50 (9H, s), 3.44-3.54 (2H, m), 3.90 (3H, s), 4.43-4.53 (2H, m), 5.12 (1H, t, J=5.6 Hz), 6.60 (1H, d, J=3.2 Hz), 7.05 (1H, d, J=8.9 Hz), 7.16-7.22 (2H, m), 7.39 (1H, t, J=8.0 Hz), 7.63 (1H, m), 7.74-7.78 (1H, m), 7.89 (1H, dd, J=2.7 Hz, 8.9 Hz), 8.03 (1H, d, J=2.7 Hz), 8.52 (1H, s), 8.61 (1H, br s).

### (ii) Production of 3-{4-[(5-{2-[(tert-butoxycarbonyl)amino]ethyl}-5H-pyrrolo[3,2-d]pyrimidin-4-yl)amino]-2-chlorophenoxy}benzoic acid

To methyl 3-{4-[(5-{2-[(tert-butoxycarbonyl)amino]ethyl}-5H-pyrrolo[3,2-d]pyrimidin-4-yl)amino]-2-chlorophenoxy}benzoate (2.96 g) were added methanol (50 mL), tetrahydrofuran (10 mL) and 1N aqueous sodium hydroxide solution (11 mL) and the mixture was stirred at 60° C. overnight. After concentration under reduced pressure, water and acetic acid (0.63 mL) were added, and the mixture was extracted with ethyl acetate. The organic layer was washed with saturated brine and dried over anhydrous magnesium sulfate. The solvent was evaporated under reduced pressure and the obtained residue was crystallized from methanol-acetonitrile-diethyl ether to give the title compound (2.58 g) as white crystals.

¹H-NMR (DMSO-d₆) δ: 1.32 (9H, s), 3.22-3.32 (2H, m), 4.51 (2H, t, J=6.2 Hz), 6.49 (1H, d, J=3.0 Hz), 7.10-7.20 (1H, m), 7.24-7.34 (3H, m), 7.52 (1H, t, J=8.0 Hz), 7.61,(1H, m), 7:67 (1H, d, J=7.7 Hz), 7.75-7.84 (1H, m), 7.97 (1H, m), 8.33 (1H, s), 8.66 (1H, br s).

### (iii) Production of 3-(4-{[5-(2-aminoethyl)-5H-pyrrolo[3,2-d]pyrimidin-4-yl]amino}-2-chlorophenoxy)-N-(tert-butyl)benzamide dihydrochloride

A mixture of 3-{4-[(5-{2-[(tert-butoxycarbonyl)amino]ethyl}-5H-pyrrolo[3,2-d]pyrimidin-4-yl)amino]-2-chlorophenoxy}benzoic acid (524 mg), tert-butylamine (0.126 mL), 1-ethyl-3-(3-dimethylaminopropyl)carbodiimide hydrochloride (230 mg), 1-hydroxybenzotriazole (184 mg) and N,N-dimethylformamide (10 mL) was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure. To the residue were added ethanol (2 mL) and 4N hydrogen chloride/ethyl acetate solution (2 mL) and the mixture was stirred at room temperature overnight. Ethyl acetate was added to the reaction mixture, and the precipitated solid was collected by filtration to give the title compound (427 mg) as a pale-yellow powder.

¹H-NMR (DMSO-d₆) δ: 1.36 (9H, s), 3.23-3.37 (2H, m), 4.96-5.06 (2H, m), 6.74 (1H, d, J=3.3 Hz), 7.17 (1H, dd, J=2.6 Hz, 8.1 Hz), 7.25 (1H, d, J=8.8 Hz), 7.35 (1H, m), 7.47 (1H, t, J=7.8 Hz), 7.58-7.66 (2H, m), 7.85 (1H, s), 7.90 (1H, m), 8.05 (1H, m), 8.27 (3H, br s), 8.74 (1H, s), 10.04 (1H, br s).

### (iv) Production of N-(tert-butyl)-3-(2-chloro-4-{[5-(2-{[(methylsulfonyl)acetyl]amino}ethyl)-5H-pyrrolo[3,2-d]pyrimidin-4-yl]amino}phenoxy)benzamide hydrochloride

A mixture of 3-(4-{[5-(2-aminoethyl)-5H-pyrrolo[3,2-d]pyrimidin-4-yl]amino}-2-chlorophenoxy)-N-(tert-butyl)benzamide dihydrochloride (166 mg), methylsulfonylacetic acid (62 mg), 1-ethyl-3-(3-dimethylaminopropyl)carbodiimide hydrochloride (86 mg), 1-hydroxybenzotriazole (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to silica gel column chromatography (eluent, methanol:ethyl acetate=0:100→15:85). The objective fractions were concentrated under reduced pressure. The residue was dissolved in ethyl acetate-ethanol and 1N hydrogen chloride/ethyl acetate solution (0.3 mL) was added. The solvent was evaporated under reduced pressure and the obtained residue was crystallized from ethanol-ethyl acetate to give the title compound (121 mg) as white crystals.

¹H-NMR (DMSO-d₆) δ: 1.36 (9H, s), 3.06 (3H, s), 3.50-3.61 (2H, m), 4.07 (2H, s), 4.67-4.77 (2H, m), 6.67 (1H, d, J=3.1 Hz), 7.13-7.20 (1H, m), 7.25 (1H, d, J=8.9 Hz), 7.37 (1H, m), 7.47 (1H, t, J=8.0 Hz), 7.60-7.69 (2H, m), 7.85 (1H, s), 7.93 (1H, d, J=2.5 Hz), 7.96 (1H, d, J=3.1 Hz), 8.74 (1H, s), 8.78-8.87 (1H, m), 9.99 (1H, br s).

### Example C-23

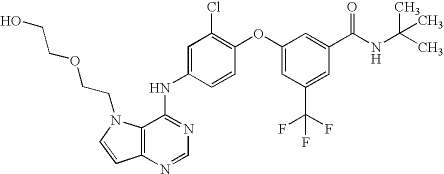

### Production of N-(tert-butyl)-3-[2-chloro-4-({5-[2-(2-hydroxyethoxy)ethyl]-5H-pyrrolo[3,2-d]pyrimidin-4-yl}amino)phenoxy]-5-(trifluoromethyl)benzamide

### (i) Production of methyl 3-hydroxy-5-(trifluoromethyl)benzoate

3-Amino-5-(trifluoromethyl)benzoic acid (2.80 g) was dissolved in concentrated sulfuric acid (50 g) and water (50 mL) and the mixture was cooled to −10° C. Water (120 mL) was added, and an aqueous solution (20 mL) of sodium nitrite (0.942 g) was added dropwise. Water (10 mL) was added, and the mixture was stirred at −10° C. for 10 min and at 0° C. for 30 min. The mixture was further stirred with heating under reflux for 1 hr. After allowing to cool, water was added to the reaction mixture, and the mixture was extracted with ethyl acetate. The organic layer was washed with saturated brine and dried over anhydrous magnesium sulfate. The solvent was evaporated under reduced pressure and the obtained residue was dissolved in methanol (30 mL). Concentrated hydrochloric acid (0.9 mL) was added and the mixture was stirred with heating under reflux overnight. The reaction mixture was concentrated under reduced pressure, aqueous sodium hydrogencarbonate solution was added to the residue, and the mixture was extracted with ethyl acetate. The ethyl acetate layer was washed with saturated brine and dried over anhydrous magnesium sulfate. The solvent was evaporated under reduced pressure and the obtained residue was subjected to silica gel column chromatography (eluent, ethyl acetate:hexane=10:90→4.30:70). The objective fractions were concentrated under reduced pressure to give the title compound (1.86 g) as a pale-yellow powder.

¹H-NMR (CDCl₃) δ: 3.95 (3H, s), 5.49 (1H, s), 7.29 (1H, m), 7.70 (1H, m), 7.87 (1H, m).

### (ii) Production of methyl 3-(4-amino-2-chlorophenoxy)-5-(trifluoromethyl)benzoate

A mixture of 2-chloro-l-fluoro-4-nitrobenzene (1.48 g), methyl 3-hydroxy-5-(trifluoromethyl)benzoate (1.86 g), potassium carbonate (1.75 g) and N,N-dimethylformamide (10 mL) was stirred at room temperature overnight. The reaction mixture was concentrated under reduced pressure, water was added and the mixture was extracted with ethyl acetate. The organic layer was washed with saturated brine, and dried over anhydrous magnesium sulfate. The solvent was evaporated under reduced pressure and the obtained residue was subjected to silica gel column chromatography (eluent, ethyl acetate:hexane=5:95→15:85). The objective fractions were concentrated under reduced pressure. To the residue were added ethyl acetate (30 mL) and 5% platinum-activated carbon (90 mg) and the mixture was stirred under a hydrogen atmosphere at room temperature for 4 hr. The catalyst was filtered off, and the filtrate was concentrated and the obtained residue was subjected to silica gel column chromatography (eluent, ethyl acetate:hexane=15:85→35:65). The objective fractions were concentrated under reduced pressure. Hexane was added to the residue, and the precipitated solid was collected by filtration to give the title compound (2.50 g) as a white powder.

¹H-NMR (CDCl₃) δ: 3.76 (2H, br s), 3.92 (3H, s), 6.61 (1H, dd, J=8.6, 2.7 Hz), 6.81 (1H, d, J=2.7 Hz), 6.94 (1H, d, J=8.6 Hz), 7.31 (1H, m), 7.64 (1H, m), 7.95 (1H, m).

### (iii) Production of methyl 3-{4-[(5-{2-[2-(benzoyloxy)ethoxy]ethyl}-5H-pyrrolo[3,2-d]pyrimidin-4-yl)amino]-2-chlorophenoxy}-5-(trifluoromethyl)benzoate

A mixture of 2-[2-(4-chloro-5H-pyrrolo[3,2-d]pyrimidin-5-yl)ethoxy]ethyl benzoate (346 mg), methyl 3-(4-amino-2-chlorophenoxy)-5-(trifluoromethyl)benzoate (346 mg) and isopropyl alcohol (5 mL) was stirred at 80° C. for 5 hr. 2-[2-(4-Chloro-5H-pyrrolo[3,2-d]pyrimidin-5-yl)ethoxy]ethyl benzoate (69 mg) was added, and the mixture was further stirred at 80° C. for 3 hr. The reaction mixture was concentrated under reduced pressure, aqueous sodium hydrogencarbonate solution was added, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure to give the title compound (609 mg) as a white powder.

¹H-NMR (CDCl₃) δ: 3.93 (3H, s), 3.95-4.00 (2H, m), 4.06-4.12 (2H, m), 4.47-4.53 (2H, m), 4.56-4.63 (2H, m), 6.64 (1H, d, J=3.3 Hz), 6.83 (1H, d, J=8.8 Hz), 7.24 (1H, d, J=3.3 Hz), 7.29-7.43 (4H, m), 7.45-7.52 (1H, m), 7.69 (1H, m), 7.77-7.83 (2H, m), 7.92 (1H, d, J=2.5 Hz), 8.00 (1H, m), 8.52 (1H, s), 8.83 (1H, br s).

### (iv) Production of 3-[2-chloro-4-({5-[2-(2-hydroxyethoxy)ethyl]-5H-pyrrolo[3,2-d]pyrimidin-4-yl}amino)phenoxy]-5-(trifluoromethyl)benzoic acid

To methyl 3-{4-[(5-{2-[2-(benzoyloxy)ethoxy]ethyl}-5H-pyrrolo[3,2-d]pyrimidin-4-yl)amino]-2-chlorophenoxy}-5-(trifluoromethyl)benzoate (609 mg) were added methanol (12 mL) and 1N aqueous sodium hydroxide solution (3 mL) and the mixture was stirred at room temperature overnight. Water and 1N hydrochloric acid (3 mL) were added to the reaction mixture, and the mixture was extracted with ethyl acetate. The organic layer was washed with saturated brine and dried over anhydrous magnesium sulfate. The solvent was evaporated under reduced pressure, ethanol-acetonitrile-diethyl ether was added to the obtained residue, and the precipitated solid was collected by filtration to give the title compound (416 mg) as a white powder.

¹H-NMR (DMSO-d₆) δ: 3.50 (4H, m), 3.85 (2H, t, J=4.4 Hz), 4.56-4.88 (1H, m), 4.68 (2H, t, J=4.4 Hz), 6.54 (1H, d, J=3.0 Hz), 7.38 (1H, d, J=8.7 Hz), 7.52 (1H, m), 7.59-7.80 (3H, m), 7.90 (1H, m), 8.05 (1H, m), 8.40 (1H, s), 9.11 (1H, br s).

### (v) Production of N-(tert-butyl)-3-[2-chloro-4-({5-[2-(2-hydroxyethoxy)ethyl]-5H-pyrrolo[3,2-d]pyrimidin-4-yl}amino)phenoxy]-5-(trifluoromethyl)benzamide

A mixture of 3-[2-chloro-4-({5-[2-(2-hydroxyethoxy)ethyl]-5H-pyrrolo[3,2-d]pyrimidin-4-yl}amino)phenoxy]-5-(trifluoromethyl)benzoic acid (322 mg), tert-butylamine (0.126 mL), 1-ethyl-3-(3-dimethylaminopropyl)carbodiimide hydrochloride (138 mg), 1-hydroxybenzotriazole (153 mg) and N,N-dimethylformamide (5 mL) was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, methanol:ethyl acetate=0:100→20:80). The objective fractions were concentrated under reduced pressure. The residue was crystallized from ethyl acetate-diethyl ether to give the title compound (247 mg) as white crystals.

¹H-NMR (CDCl₃) δ: 1.46 (9H, s), 3.70-4.82 (4H, m), 4.02 (2H, t, J=4.4 Hz), 4.57 (2H, t, J=4.4Hz), 6.00 (1H, br s), 6.61 (1H, d, J=3.2 Hz), 7.06 (1H, d, J=8.8 Hz), 7.21 (1H, d, J=3.2 Hz), 7.29 (1H, m), 7.44 (1H, m), 7.57 (1H, m), 7.62 (1H, dd, J=2.8,Hz, 8.8 Hz), 7.93 (1H, d, J=2.8 Hz), 8.51 (1H, s), 8.87 (1H, br s).

### Example C-24

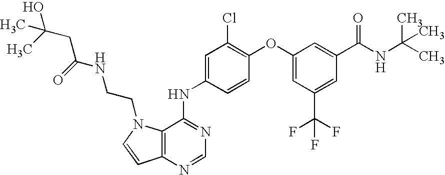

### Production of N-(tert-butyl)-3-{2-chloro-4-[(5-{2-[(3-hydroxy-3-methylbutanoyl)amino]ethyl}-5H-pyrrolo[3,2-d]pyrimidin-4-yl)amino]phenoxy}-5-(trifluoromethyl)benzamide

### (i) Production of methyl 3-{4-[(5-{2-[(tert-butoxycarbonyl)amino]ethyl}-5H-pyrrolo[3,2-d]pyrimidin-4-yl)amino]-2-chlorophenoxy}-5-(trifluoromethyl)benzoate

A mixture of tert-butyl[2-(4-chloro-5H-pyrrolo[3,2-d]pyrimidin-5-yl)ethyl]carbamate (1.48 g), methyl 3-(4-amino-2-chlorophenoxy)-5-(trifluoromethyl)benzoate (1.73 g) and isopropyl alcohol (20 mL) was stirred at 80° C. for 5 hr. tert-Butyl[2-(4-chloro-5H-pyrrolo[3,2-d]pyrimidin-5-yl)ethyl]carbamate (0.30 g) was added and the mixture was further stirred at 80° C. for 3 hr. The reaction mixture was concentrated under reduced pressure, aqueous sodium hydrogencarbonate solution was added and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure. To the residue were added acetone and diisopropyl ether, and the precipitated solid was collected by filtration to give the title compound (2.06 g) as a white powder.

¹H-NMR (CDCl₃) δ: 1.50 (9H, s), 3.45-3.55 (2H, m), 3.93 (3H, s), 4.44-4.54 (2H, m), 5.10-5.18 (1H, m), 6.61 (1H, d, J=3.2 Hz), 7.11 (1H, d, J=8.9 Hz), 7.20 (1H, d, J=3.2 Hz), 7.39 (1H, m), 7.77 (1H, m), 7.96 (1H, dd, J=8.9, 2.5 Hz), 8.00 (1H, s), 8.07 (1H, d, J=2.5 Hz), 8.53 (1H, s), 8.67 (1H, br s).

### (ii) Production of 3-{4-[(5-{2-[(tert-butoxycarbonyl)amino]ethyl}-5H-pyrrolo[3,2-d]pyrimidin-4-yl)amino]-2-chlorophenoxy}-5-(trifluoromethyl)benzoic acid

To methyl 3-{4-[(5-{2-[(tert-butoxycarbonyl)amino]ethyl}-5H-pyrrolo[3,2-d]pyrimidin-4-yl)amino]-2-chlorophenoxy}-5-(trifluoromethyl)benzoate (2.42 g) were added methanol (10 mL), tetrahydrofuran (10 mL) and 1N aqueous sodium hydroxide solution (8 mL) and the mixture was stirred at room temperature overnight. To the reaction mixture were added water and 1N hydrochloric acid (8 mL), and the mixture was extracted with ethyl acetate. The organic layer was washed with saturated brine and dried over anhydrous magnesium sulfate. The solvent was evaporated under reduced pressure and the obtained residue was crystallized from methanol-diethyl ether to give the title compound (2.03 g) as white crystals.

¹H-NMR (DMSO-d₆) δ: 1.32 (9H, s), 3.20-3.36 (2H, m), 4.53 (2H, t, J=6.4 Hz), 6.51 (1H, d, J=3.0 Hz), 7.10-7.23 (1H, m), 7.39 (1H, d, J=8.7 Hz), 7.54 (1H, m), 7.63 (2H, m), 7.80-7.90 (1H, m), 7.90 (1H, m), 8.02 (1H, m), 8.35 (1H, s), 8.73 (1H, br s).

### (iii) Production of 3-(4-([5-(2-aminoethyl)-5H-pyrrolo[3,2-d]pyrimidin-4-yl]amino1-2-chlorophenoxy)-N-(tert-butyl)-5-(trifluoromethyl)benzamide dihydrochloride

A mixture of 3-{4-[(5-{2-[(tert-butoxycarbonyl)amino]ethyl}-5H-pyrrolo[3,2-d]pyrimidin-4-yl)amino]-2-chlorophenoxy}-5-(trifluoromethyl)benzoic acid (888 mg), tert-butylamine (0.189 mL), 1-ethyl-3-(3-dimethylaminopropyl)carbodiimide hydrochloride (345 mg), 1-hydroxybenzotriazole (276 mg) and N,N-dimethylformamide (10 mL) was stirred at room temperature overnight. Water was, added to the reaction mixture, and the mixture was extracted with ethyl acetate. The ethyl acetate layer was washed with saturated brine and dried over anhydrous magnesium sulfate. The solvent was evaporated under reduced pressure and the obtained residue was subjected to silica gel column chromatography (eluent, ethyl acetate:hexane=70:30→100:0→methanol:ethyl acetate=10:90). The objective fractions were concentrated under reduced pressure. To the residue were added ethanol (1 mL) and 4N hydrogen chloride/ethyl acetate solution (5 mL) and the mixture was stirred at room temperature for 2 hr. The reaction mixture was concentrated under reduced pressure, ethanol and diethyl ether were added, and the precipitated solid was collected by filtration to give the title compound (815 mg) as a white powder.

¹H-NMR (DMSO-d₆) δ: 1.37 (9H, s), 3.23-3..38 (2H, m), 5.06 (2H, m), 6.76 (1H, d, J=3.0 Hz), 7.37-7.44 (1H, m), 7.52 (1H, m), 7.59 (1H, m), 7.66-7.75 (1H, m), 7.94-8.55 (7H, m), 8.76 (1H, s), 9.95-10.04 (1H, m).

### (iv) Production of N-(tert-butyl)-3-{2-chloro-4-[(5-{2-[(3-hydroxy-3-methylbutanoyl)amino]ethyl}-5H-pyrrolo[3,2-d]pyrimidin-4-yl)amino]phenoxy}-5-(trifluoromethyl)benzamide

A mixture of 3-(4-{[5-(2-aminoethyl)-5H-pyrrolo[3,2-d]pyrimidin-4-yl]amino}-2-chlorophenoxy)-N-(tert-butyl)-5-(trifluoromethyl)benzamide dihydrochloride (186 mg), 3-hydroxy-3-methylbutanoic acid (53 mg), 1-ethyl-3-(3-dimethylaminopropyl)carbodiimide hydrochloride (86 mg), 1-hydroxybenzotriazole (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to silica gel column chromatography (eluent, methanol:ethyl acetate=0:100→10:90). The objective fractions were concentrated under reduced pressure. The residue was crystallized from ethyl acetate-diisopropyl ether to give the title compound (152 mg) as white crystals.

¹H-NMR (CDCl₃) δ: 1.31 (6H, s), 1.48 (9H, s), 2.48 (2H, s), 3.54-3.66 (2H, m), 4.41-4.53 (2H, m), 6.01 (1H, br s), 6.57 (1H, d, J=3.3 Hz), 7.07 (1H, d, J=9.0 Hz), 7.18 (1H, d, J=3.3 Hz), 7.25-7.35 (2H, m), 7.48 (1H, m), 7.62 (1H, m), 7.78 (1H, dd, J=2.4 Hz, 9.0 Hz), 8.10 (1H, d, J=2.4 Hz), 8.49 (1H, s), 8.72 (1H, br s).

### Example C-25

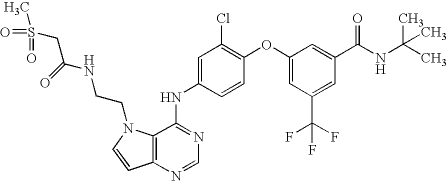

### Production of N-(tert-butyl)-3-(2-chloro-4-{[5-(2-{[(methylsulfonyl)acetyl]amino}ethyl)-5H-pyrrolo[3,2-d]pyrimidin-4-yl]amino}phenoxy)-5-(trifluoromethyl)benzamide

A mixture of 3-(4-{[5-(2-aminoethyl)-5H-pyrrolo[3,2-d]pyrimidin-4-yl]amino}-2-chlorophenoxy)-N-(tert-butyl)-5-(trifluoromethyl)benzamide dihydrochloride (186 mg), methylsulfonylacetic acid (62 mg), 1-ethyl-3-(3-dimethylaminopropyl)carbodiimide hydrochloride (86 mg), 1-hydroxybenzotriazole (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to silica gel column chromatography (eluent, methanol:ethyl acetate=0:100→15:85) and basic silica gel column chromatography (eluent, methanol:ethyl acetate=0:100→10:90). The objective fractions were concentrated under reduced pressure. The residue was crystallized from ethyl acetate-diisopropyl ether to give the title compound (146 mg) as white crystals.

¹H-NMR (CDCl₃) δ: 1.48 (9H, s), 3.14 (3H, s), 3.60-3.74 (2H, m), 4.00 (2H, s), 4.40-4.54 (2H, m), 6.06 (1H, br s), 6.58 (1H, d, J=3.0 Hz), 7.08 (1H, d, J=8.8 Hz), 7.21 (1H, d, J=3.0 Hz), 7.35 (1H, m), 7.46 (1H, m), 7.58 (1H, m), 7.78 (1H, dd, J=2.3 Hz, 8.8 Hz), 7.87-7.96 (1H, m), 7.97 (1H, d, J=2.3 Hz), 8.29 (1H, br s), 9.46 (1H, s).

### Example C-26

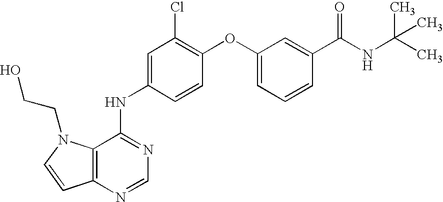

### Production of N-(tert-butyl)-3-(2-chloro-4-{[5-(2-hydroxyethyl)-5H-pyrrolo[3,2-d]pyrimidin-4-yl]amino}phenoxy)benzamide

### (i) Production of 3-(2-chloro-4-{[5-(2-hydroxyethyl)-5H-pyrrolo[3,2-d]pyrimidin-4-yl]amino}phenoxy)benzoic acid

A mixture of 2-(4-chloro-5H-pyrrolo[3,2-d]pyrimidin-5-yl)ethyl benzoate (1.51 g), methyl 3-(4-amino-2-chlorophenoxy)benzoate (1.39 g) and isopropyl alcohol (20 mL) was stirred at 80° C. overnight. Aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure. To the residue were added methanol (30 mL) and 1N aqueous sodium hydroxide solution (13.5 mL) and the mixture was stirred at room temperature for 3 days. The reaction mixture was concentrated under reduced pressure, water and 1N hydrochloric acid (13.5 mL) were added, and the mixture was extracted with ethyl acetate-tetrahydrofuran. The extract was washed with water and the solvent was evaporated under reduced pressure. The residue was crystallized from acetonitrile-diethyl ether to give the title compound (1.88 g) as white crystals.

¹H-NMR (DMSO-d₆) δ: 3.88 (2H, m), 4.50-4.60 (2H, m), 6.31 (1H, br s), 6.52 (1H, d, J=3.0 Hz), 7.23-7.34 (3H, m), 7.51 (1H, t, J=8.0 Hz), 7.59-7.71 (3H, m), 7.99 (1H, d, J=2.7 Hz), 8.35 (1H, s), 9.90 (1H, br s).

### (ii) Production of N-(tert-butyl)-3-(2-chloro-4-{[5-(2-hydroxyethyl)-5H-pyrrolo[3,2-d]pyrimidin-4-yl]amino}phenoxy)benzamide

A mixture of 3-(2-chloro-4-{[5-(2-hydroxyethyl)-5H-pyrrolo[3,2-d]pyrimidin-4-yl]amino}phenoxy)benzoic acid (850 mg), tert-butylamine (0.420 mL), 1-ethyl-3-(3-dimethylaminopropyl)carbodiimide hydrochloride (466 mg), 1-hydroxybenzotriazole (368 mg) and N,N-dimethylformamide (10 mL) was stirred at room temperature overnight. The reaction mixture was concentrated under reduced pressure, water was added and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, methanol:ethyl acetate=0:100→20:80) and basic silica gel column chromatography (eluent, methanol:ethyl acetate=0:100→20:80). The objective fractions were concentrated under reduced pressure. The residue was crystallized from ethyl acetate-diisopropyl ether to give the title compound (630 mg) as white crystals.

¹H-NMR (CDCl₃) δ: 1.45 (9H, s), 1.66 (1H, br s), 4.08-4.16 (2H, m), 4.35-4.42 (2H, m), 5.99 (1H, br s), 6.16 (1H, d, J=3.3 Hz), 6.98-7.03 (2H, m), 7.04-7.12 (1H, m), 7.30-7.37 (3H, m), 7.41 (1H, dd, J=2.6 Hz, 8.8 Hz), 7.80 (1H, d, J=2.6 Hz), 8.23 (1H, s), 9.68 (1H, br s).

### Example C-27

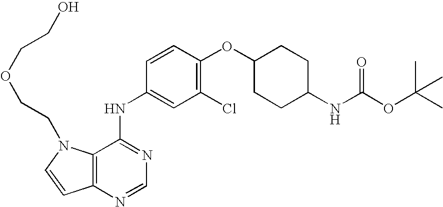

### Production of tert-butyl{4-[2-chloro-4-({5-[2-(2-hydroxyethoxy)ethyl]-5H-pyrrolo[3,2-d]pyrimidin-4-yl}amino)phenoxy]cyclohexyl}carbamate

A mixture of 2-[2-(4-chloro-5H-pyrrolo[3,2-d]pyrimidin-5-yl)ethoxy]ethyl benzoate (300 mg), tert-butyl[4-(4-amino-2-chlorophenoxy)cyclohexyl]carbamate (384 mg) and isopropyl alcohol (7.0 mL) was stirred at 80° C. overnight. The reaction mixture was concentrated under reduced pressure, water and saturated aqueous sodium hydrogencarbonate were added and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:methanol=100:0→80:20). The objective fractions were concentrated under reduced pressure. The crude product was dissolved in methanol (5.0 mL) and tetrahydrofuran (1.0 mL), 1N aqueous sodium hydroxide solution (2.5 mL) was added and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate/hexane to give the title compound (163 mg) as a white powder.

¹H-NMR (DMSO-d₆) δ: 1.20-1.52 (4H, m), 1.38 (9H, s), 1.82-2.14 (4H, m), 3.25-3.32 (4H, m), 3.81 (2H, t, J=4.9 Hz), 4.15-4.29 (1H, m), 4.60-4.72 (3H, m), 6.48 (1H, d, J=3 Hz), 6.80-6.83 (1H, m), 7:17 (1H, d, J=9 Hz), 7.46-7.49 (1H, m), 7.63 (1H, d, J=3 Hz), 7.77 (1H, d, J=3 Hz), 8.26 (1H, s), 8.68 (1H, br s).

### Example C-28

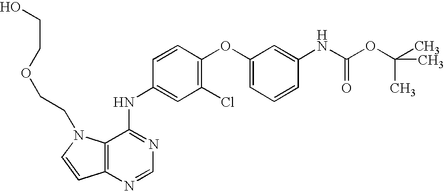

### Production of tert-butyl{3-[2-chloro-4-({5-[2-(2-hydroxyethoxy)ethyl]-5H-pyrrolo[3,2-d]pyrimidin-4-yl}amino)phenoxy]phenyl}carbamate

Using 2-[2-(4-chloro-5H-pyrrolo[3,2-d]pyrimidin-5-yl)ethoxy]ethyl benzoate (165 mg), tert-butyl[3-(4-amino-2-chlorophenoxy)phenyl]carbamate (200 mg), isopropyl alcohol (7.0 mL), methanol (5.0 mL), tetrahydrofuran (1.0 mL) and 1N aqueous sodium hydroxide solution (2.5 mL) and in the same manner as in Example C-27, the title compound (90 mg) was obtained as crystals.

¹H-NMR (DMSO-d₆) δ: 1.45 (9H, s), 3.49 (4H, s), 3.83 (2H, t, J=4.7 Hz), 4.63-4.72 (3H, m), 6.51 (2H, d, J=3 Hz), 7.12-7.24 (4H, m), 7.63-7.69 (2H, m), 7.99 (1H, s), 8.34 (1H, s), 8.93 (1H; s), 9.43 (1H, s).

### Example C-29

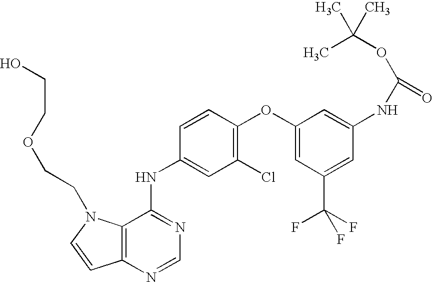

### Production of tert-butyl[3-[2-chloro-4-({5-[2-(2-hydroxyethoxy)ethyl]-5H-pyrrolo[3,2-d]pyrimidin-4-yl}amino)phenoxy]-5-(trifluoromethyl)phenyl]carbamate

Using 2-[2-(4-chloro-5H-pyrrolo[3,2-d]pyrimidin-5-yl)ethoxy]ethyl benzoate (206 mg), tert-butyl[3-(4-amino-2-chlorophenoxy)-5-(trifluoromethyl)phenyl]carbamate (300 mg), isopropyl alcohol (10 mL), methanol (2.0 mL), tetrahydrofuran (1.0 mL) and 1N aqueous sodium hydroxide solution (2.0 mL) and in the same manner as in Example C-27, the title compound (53 mg) was obtained as crystals.

¹H-NMR (CDCl₃) δ: 1.53 (9H, s), 3.72-3.82 (5H, m), 4.02 (2H, t, J=4.9 Hz), 4.58 (2H, t, J=4.9 Hz), 6.36 (1H, d, J=2.1 Hz), 6.58 (2H, d, J=4.7 Hz), 6.66 (1H, d, J=3.2 Hz), 7.07 (1H, d, J=8.9 Hz), 7.23-7.25 (2H, m), 7.62 (1H, dt, J=8.9, 2.1 Hz), 7.87 (1H, s), 8.54 (1H, s), 8.79 (1H, s).

### Example C-30

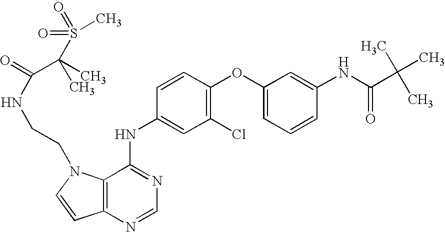

### Production of N-[3-(2-chloro-4-{[5-(2-{[2-methyl-2-(methylsulfonyl)propanoyl]amino}ethyl)-5H-pyrrolo[3,2-d]pyrimidin-4-yl]amino}phenoxy)phenyl]-2,2-dimethylpropanamide

### (i) Production of 5-(2-aminoethyl)-N-[4-(3-aminophenoxy)-3-chlorophenyl]-5H-pyrrolo[3,2-d]pyrimidin-4-amine trihydrochloride

A solution of tert-butyl[-(4-chloro-5H-pyrrolo[3,2-d]pyrimidin-5-yl)ethyl]carbamate (267 mg) and tert-butyl[3-(4-amino-2-chlorophenoxy)phenyl]carbamate (430 mg) in 1-methyl-2-pyrrolidone (15 mL) was stirred at 120° C. for 5 hr. After cooling to room temperature, water was added to the reaction mixture and the mixture was extracted with ethyl acetate. The organic layer was washed successively with water and saturated brine, and dried over magnesium sulfate. The residue was separated and purified by silica gel column chromatography (hexane:ethyl acetate=19:1→3:2-3ethyl acetate) to give a brown solid. To a solution of the obtained solid in tetrahydrofuran (20 mL) was added 2N hydrochloric acid (10 mL) at room temperature, and the mixture was stirred at 60° C. for 20 hr. After concentration under reduced pressure, ethanol was added and the mixture was further concentrated. Diisopropyl ether was added to the residue and the resulting crystals were collected by filtration. The crystals were washed with diisopropyl ether to give the title compound (342 mg) as pale-yellow crystals.

¹H-NMR (DMSO-d₆) δ: 3.26-3.34 (2H, m), 5.04-5.13 (2H, m), 6.76-7.05 (4H, m), 7.28-7.69 (3H, m), 7.94 (1H, s), 8.11 (1H, s), 8.45 (3H, br s), 8.76 (1H, s), 10.39 (2H, br s).

### (ii) Production of N-[3-(2-chloro-4-{[5-(2-{[2-methyl-2-(methylsulfonyl)propanoyl]amino}ethyl)-5H-pyrrolo[3,2-d]pyrimidin-4-yl]amino}phenoxy)phenyl]-2,2-dimethylpropanamide

A mixture of 5-(2-aminoethyl)-N-[4-(3-aminophenoxy)-3-chlorophenyl]-5H-pyrrolo[3,2-d]pyrimidin-4-amine trihydrochloride (114 mg), 2-methyl-2-(methylsulfonyl)propanoic acid (210 mg), 1-ethyl-3-(3-dimethylaminopropyl)carbodiimide hydrochloride (380 mg), 1-hydroxybenzotriazole (41 mg), triethylamine (1.0 mL) and tetrahydrofuran (5.0 mL) was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, methanol:ethyl acetate=0:100→10:90). The objective fractions were concentrated under reduced pressure. The obtained crude product was dissolved in tetrahydrofuran (5.0 mL), N-methylmorpholine (1.0 mL) and 2,2-dimethylpropanoyl chloride (0.25 mL) were added, and the mixture was stirred for 1 hr. Under ice-cooling, saturated aqueous sodium hydrogencarbonate was added, and the mixture was extracted with dichloromethane. The extract was dried over magnesium sulfate and concentrated, and the residue was separated and purified by silica gel column chromatography (eluent, ethyl acetate:methanol=100:0→ethyl acetate:methanol=80:20), and crystallized from diethyl ether/ethyl acetate to give the title compound (82 mg) as crystals.

¹H-NMR (DMSO-d₆) δ: 1.19 (9H, s), 1.14 (6H, s), 2.96 (3H, s), 3.39-3.50 (2H, m), 4.55-4.59 (2H, m), 6.49 (1H, d, J=3.2 Hz), 6.66-6.68 (1H, m), 7.17-7.74 (6H, m), 7.97 (1H, s), 8.22 (1H, br s), 8.34 (1H, s), 8.65 (1H, s), 9.26 (1H, s).

### Example C-31

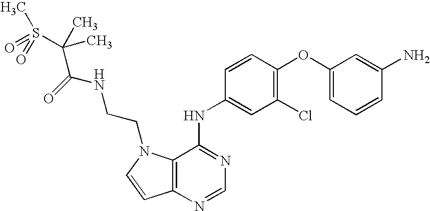

### Production of N-[2-(4-{[4-(3-aminophenoxy)-3-chlorophenyl]amino}-5H-pyrrolo[3,2-d]pyrimidin-5-yl)ethyl]-2-methyl-2-(methylsulfonyl)propanamide

A mixture of 5-(2-aminoethyl)-N-[4-(3-aminophenoxy)-3-chlorophenyl]-5H-pyrrolo[3,2-d]pyrimidin-4-amine trihydrochloride (860 mg), 2-methyl-2-(methylsulfonyl)propanoic acid (700 mg), 1-ethyl-3-(3-dimethylaminopropyl)carbodiimide hydrochloride (1.10 g), 1-hydroxybenzotriazole (50 mg), triethylamine (3.0 mL) and tetrahydrofuran (30 mL) was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel, column chromatography (eluent, methanol:ethyl acetate=0:100→10:90). The objective fractions were concentrated under reduced pressure. The obtained crude product was crystallized from diethyl ether/ethyl acetate to give the title compound (850 mg).

¹H-NMR (DMSO-d₆) δ: 1.41(6H, s), 2.96 (3H, s), 3.39-3.51 (2H, m), 4.54-4.59 (2H, m), 5.32 (2H, m), 6.07-6.11 (2H, m), 6.29 (1H, d, J=8.8 Hz), 6.53 (1H, d, J=3.0 Hz), 6.94-6.71 (1H, m), 7.11 (1H, d, J=8.8 Hz), 7.56-7.60 (2H, m), 7.70 (1H, d, J=3.0 Hz), 8.10 (1H, br s), 8.34 (1H, s), 8.91 (1H, s).

### Example C-32

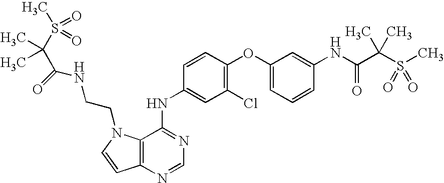

### Production of N-[3-(2-chloro-4-{[5-(2-{[2-methyl-2-(methylsulfonyl)propanoyl]amino}ethyl)-5H-pyrrolo[3,2-d]pyrimidin-4-yl]amino}phenoxy)phenyl]-2-methyl-2-(methylsulfonyl)propanamide

Using 5-(2-aminoethyl)-N-[4-(3-aminophenoxy)-3-chlorophenyl]-5H-pyrrolo[3,2-d]pyrimidin-4-amine trihydrochloride (80 mg), 2-methyl-2-(methylsulfonyl)propanoic acid (87 mg), 1-ethyl-3-(3-dimethylaminopropyl)carbodiimide hydrochloride (210 mg), 1-hydroxybenzotriazole (71 mg), triethylamine (0.9 mL) and tetrahydrofuran (8.0 mL) and in the same manner as in Example C-31, the title compound (89 mg) was obtained as crystals.

¹H-NMR (DMSO-d₆) δ: 1.41 (6H, s), 1.64 (6H, s), 2.96 (3H, s), 3.03 (3H, s), 3.41-3.52 (2H, m), 4.55-4.59 (2H, m), 6.49 (1H, d, J=3.2 Hz), 6.73-6.76 (1H, m), 7.17-7.76 (6H, m), 7.99 (1H, d. J=3.2 Hz), 8.22 (1H, br s), 8.34 (1H, s), 8.66 (1H, s), 9.52 (1H, s).

### Example C-33

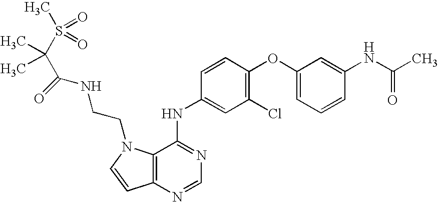

### Production of N-{2-[4-({4-[3-(acetylamino)phenoxy]-3-chlorophenyl}amino)-5H-pyrrolo[3,2-d]pyrimidin-5-yl]ethyl}-2-methyl-2-(methylsulfonyl)propanamide

A mixture of N-[2-(4-{[4-(3-aminophenoxy)-3-chlorophenyl]amino}-5H-pyrrolo[3,2-d]pyrimidin-5-yl)ethyl]-2-methyl-2-(methylsulfonyl)propanamide (91 mg), triethylamine (0.2 mL), acetic anhydride (0.3 mL) and tetrahydrofuran (7.0 mL) was stirred at room temperature for 1 hr. Under ice-cooling, saturated aqueous sodium hydrogencarbonate was added, and the mixture was extracted with dichloromethane. The extract was dried over magnesium sulfate and concentrated, and the residue was separated and purified by silica gel column chromatography (eluent, ethyl acetate:methanol=100:0→380:20), and crystallized from diethyl ether/ethyl acetate to give the title compound (84 mg) as crystals.

¹H-NMR (DMSO-d₆) δ: 1.41(6H, s), 2.00 (3H, s), 2.96 (3H, s), 3.39-3.50 (2H, m), 4.55-4.59 (2H, m), 6.49 (1H, d, J=3.2 Hz), 6.63-6.68 (1H, m), 7.23-7.30 (4H, m), 7.59 (1H, s), 7.72-7.75 (1H, m), 7.96 (1H, s), 8.20 (1H, br s), 8.34 (1H, s), 8.65 (1H, s), 10.00 (1H, s).

### Example C-34

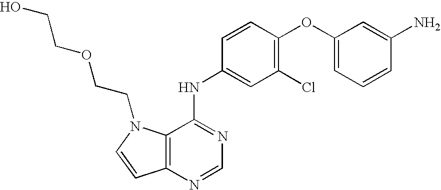

### Production of 2-[2-(4-{[4-(3-aminophenoxy)-3-chlorophenyl]amino}-5H-pyrrolo[3,2-d]pyrimidin-5-yl)ethoxy]ethanol

tert-Butyl{3-[2-chloro-4-({5-[2-(2-hydroxyethoxy)ethyl]-5H-pyrrolo[3,2-d]pyrimidin-4-yl}amino)phenoxy]phenyl}carbamate (120 mg) was dissolved in methanol (7.0 mL), 4N hydrogen chloride/ethyl acetate solution (8.0 mL) was added and the mixture was stirred for 5 hr. 8N aqueous sodium hydroxide solution (8.0 mL) and water (10 mL) were added, and the mixture was extracted with dichloromethane. The extract was dried over magnesium sulfate and concentrated, and the residue was separated and purified by silica gel column chromatography (eluent, ethyl acetate:methanol=100:0→80:20), and crystallized from diethyl ether/ethyl acetate to give: the title compound (59 mg) as crystals.

¹H-NMR (DMSO-d₆) δ: 3.49 (4H, s), 3.81-3.85 (2H, m), 4.62-4.65 (2H, t, J=4.8Hz), 4.71 (1H, m), 5.32 (2H, m), 6.06-6.09 (2H, m), 6.27 (1H, d, J=8.8 Hz), 6.51 (1H, d, J=3.0 Hz), 6.94-6.70 (1H, m), 7.11 (1H, d, J=8.8 Hz), 7.56-7.60 (1H, m), 7.70 (1H, d, J=3.0 Hz), 7.95 (1H, s), 8.34 (1H, s), 8.93 (1H, s).

### Example C-35

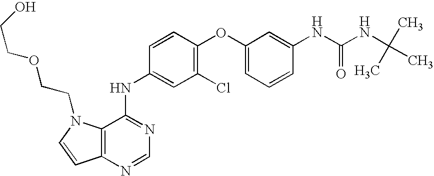

### Production of N-(tert-butyl)-N′-{3-[2-chloro-4-({5-[2-(2-hydroxyethoxy)ethyl]-5H-pyrrolo[3,2-d]pyrimidin-4-yl}amino)phenoxy]phenyl}urea

### (i) Production of 2-[2-(4-{[4-(3-aminophenoxy)-3-chlorophenyl]amino}-5H-pyrrolo[3,2-d]pyrimidin-5-yl)ethoxy]ethyl benzoate dihydrochloride

A mixture of 2-[2-(4-chloro-5H-pyrrolo[3,2-d]pyrimidin-5-yl)ethoxy]ethyl benzoate (206 mg), tert-butyl[3-(4-amino-2-chlorophenoxy)phenyl]carbamate (300 mg) and isopropyl alcohol (7.0 mL) was stirred at 80° C. for 12 hr. The reaction mixture was concentrated under reduced pressure, water and saturated aqueous sodium hydrogencarbonate were added and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, hexane:ethyl acetate=80:20→40:100). The objective fractions were concentrated under reduced pressure. The crude product was dissolved in methanol (8.0 mL), and using 4N hydrogen chloride/ethyl acetate solution (8.0 mL) and in the same manner as in Example C-34, the title compound (370 mg) was obtained as crystals.

¹H-NMR (DMSO-d₆) δ: 3.76-3.80 (2H, m), 3.87-3.94 (2H, m), 4.22-4.35 (2H, m), 4.85-4.93 (2H, m), 6.62-6.77 (3H, m), 6.87-7.18 (3H, m), 7.30-7.71 (8H, m), 7.90 (1H, s), 8.01 (1H, s), 8.65 (1H, s).

### (ii) Production of N-(tert-butyl)-N′-{3-[2-chloro-4-({5-[2-(2-hydroxyethoxy)ethyl]-5H-pyrrolo[3,2-d]pyrimidin-4-yl}amino)phenoxy]phenyl}urea

2-[2-(4-{[4-(3-Aminophenoxy)-3-chlorophenyl]amino}-5H-pyrrolo[3,2-d]pyrimidin-5-yl)ethoxy]ethyl benzoate dihydrochloride (200 mg) was suspended in toluene (10 mL), triethylamine (0.9 mL) and 2-isocyanato-2-methylpropane (0.4 mL) were added, and the mixture was to stirred at 120° C. for 6 hr. Water was added to the reaction mixture, and the mixture was extracted with ethyl acetate. The organic layer was washed with saturated brine and dried over anhydrous magnesium sulfate. The solvent was evaporated under reduced is pressure and the obtained residue was subjected to basic silica gel column chromatography (eluent, methanol:ethyl acetate=0:100→15:85). The objective fractions were concentrated under reduced pressure. The residue was dissolved in methanol (6.0 mL) and tetrahydrofuran (6.0 mL). 1N Aqueous sodium hydroxide solution (3.0 mL) was added and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate-hexane to give the title compound (74 mg) as white crystals.

¹H-NMR (DMSO-d₆) δ: 1.26 (9H, s), 3.49 (4H, s), 3.83 (2H, t, J=6.0 Hz), 4.64 (2H, t, J=6.0 Hz), 4.71 (1H, m), 5.93 (1H, s), 6.43-6.52 (2H, m), 6.96-7.21 (4H, m), 7.60-7.69 (2H, m), 7.98 (1H, d, J=3.0 Hz), 8.34 (1H, s), 8.35 (1H, s), 8.92 (1H, s).

### Example C-36

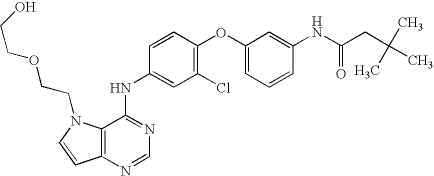

### Production of N-{3-[2-chloro-4-({5-[2-(2-hydroxyethoxy)ethyl]-5H-pyrrolo[3,2-d]pyrimidin-4-yl}amino)phenoxy]phenyl}-3,3-dimethylbutanamide

A mixture of 2-[2-(4-{[4-(3-aminophenoxy)-3-chlorophenyl]amino}-5H-pyrrolo[3,2-d]pyrimidin-5-yl)ethoxy]ethyl benzoate dihydrochloride (270 mg), triethylamine (1.9 mL), 3,3-dimethylbutanoyl chloride (0.3 mL) and tetrahydrofuran (20 mL) was stirred at room temperature for 1 hr. Under ice-cooling, saturated aqueous sodium hydrogencarbonate was added, and the mixture was extracted with dichloromethane. The extract was dried over magnesium sulfate and concentrated. The residue was subjected to silica gel column chromatography (eluent, ethyl acetate:methanol=100:0→80:20). The objective fractions were concentrated under reduced pressure. The residue was dissolved in methanol (6.0 mL) and tetrahydrofuran (6.0 mL). 1N Aqueous sodium hydroxide solution (2.0 mL) was added and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were _concentrated under reduced pressure. The residue was crystallized from ethyl acetate-hexane to give the title compound (126 mg) as white crystals.

¹H-NMR (DMSO-d₆) δ: 1.00 (9H, s), 2.15 (2H, s), 3.49 (4H, s), 3.84 (2H, t, J=6.0 Hz), 4.66 (2H, t, J=6.0 Hz), 4.71 (1H, m), 6.52 (1H, d, J=3.0 Hz), 6.68-6.70 (1H, m), 7.16-7.37 (4H, m), 7.61-7.69 (2H, m), 7.99 (1H, d, J=3.0 Hz), 8.34 (1H, s), 8.94 (1H, s), 9.85 (1H, s).

### Example C-37

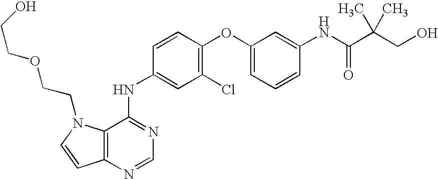

### Production of N-{3-[2-chloro-4-({5-[2-(2-hydroxyethoxy)ethyl]-5H-pyrrolo[3,2-d]pyrimidin-4-yl}amino)phenoxy]phenyl}-3-hydroxy-2,2-dimethylpropanamide

A mixture of 2-[2-(4-{[4-(3-aminophenoxy)-3-chlorophenyl]amino}-5H-pyrrolo[3,2-d]pyrimidin-5-yl)ethoxy]ethyl benzoate dihydrochloride (260 mg), 3-hydroxy-2,2-dimethylpropanoic acid (200 mg), 1-ethyl-3-(3-dimethylaminopropyl)carbodiimide hydrochloride (621 mg), 1-hydroxybenzotriazole (70 mg), triethylamine (2.0 mL) and tetrahydrofuran (15 mL) was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, methanol:ethyl acetate=0:100→10:90). The objective fractions were concentrated under reduced pressure. The obtained crude product was dissolved in methanol (6.0 mL) and tetrahydrofuran (6.0 mL), 1N aqueous sodium hydroxide solution (2.0 mL) was added, and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate-hexane to give the title compound (84 mg) as white crystals.

¹H-NMR (DMSO-d₆) δ: 1.30 (6H, s), 3.49 (4H, s), 3.56 (2H, br s), 3.84 (2H, t, J=6.0 Hz), 4.64 (2H, t, J=6.0 Hz), 4.71 (1H, s), 4.73 (1H, m), 6.50 (1H, d, J=3.2 Hz), 6.64 (1H, d, J=7.7 Hz), 7.15-7.38 (4H, m), 7.62-7.70 (2H, m), 7.99 (1H, d, J=3.2 Hz), 8.34 (1H, s), 8.94 (1H, s), 9.90 (1H, s).

### Example C-38

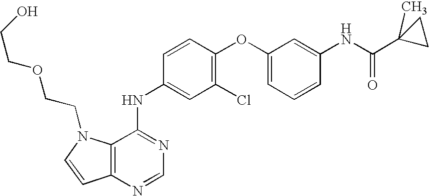

### Production of N-{3-[2-chloro-4-({5-[2-(2-hydroxyethoxy)ethyl]-5H-pyrrolo[3,2-d]pyrimidin-4-yl}amino)phenoxy]phenyl}-1-methylcyclopropanecarboxamide

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (200 mg), 1-methylcyclopropanecarboxylic acid (179 mg), 1-ethyl-3-(3-dimethylaminopropyl)carbodiimide hydrochloride (600 mg), 1-hydroxybenzotriazole (70 mg), triethylamine (2.3 mL), tetrahydrofuran (20 mL), 1N aqueous sodium hydroxide solution (2.0 mL), tetrahydrofuran (6.0 mL) and ethanol (6.0 mL) and in the same manner as in Example C-37, the title compound (69 mg) was obtained as crystals.

¹H-NMR (DMSO-d₆) δ: 0.59-0.62 (2H, m), 1.04-1.08 (2H, m), 1.37 (3H, s), 3.49 (4H, s), 3.84 (2H, t, J=6.0 Hz), 4.66 (2H, t, J=6.0 Hz), 4.71 (1H, m), 6.52 (1H, d, J=3.0 Hz), 6.60-6.70 (1H, m), 7.15 (1H, d, J=6.0 Hz), 7.23-7.41 (3H, m), 7.60-7.64 (1H, m), 7.70 (1H, d, J=3.0 Hz), 7.98 (1H, d, J=3.0 Hz), 8.36 (1H, s), 8.98 (1H, s), 9.22 (1H, s).

### Example C-39

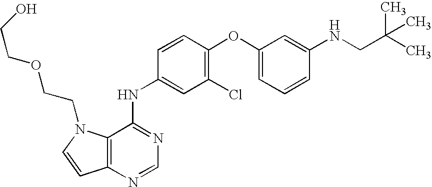

### Production of 2-(2-{4-[(3-chloro-4-{3-[(2,2-dimethylpropyl)amino]phenoxy}phenyl)amino]-5H-pyrrolo[3,2-d]pyrimidin-5-yl}ethoxy)ethanol

2-[2-(4-{[4-(3-Aminophenoxy)-3-chlorophenyl]amino}-5H-pyrrolo[3,2-d]pyrimidin-5-yl)ethoxy]ethyl benzoate dihydrochloride (220 mg) was suspended in dichloromethane (7.0 mL), and acetic acid (0.7 mL), molecular sieves 4A (300 mg) and pivalaldehyde (120 mg) were added, and the mixture was stirred for 30. min. Sodium triacetoxyborohydride (470 mg) was added, and the mixture was further stirred for 3 hr. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→15:85). The objective fractions were concentrated under reduced pressure. The residue was dissolved in methanol (6.0 mL) and tetrahydrofuran (6.0 mL). 1N Aqueous sodium hydroxide solution (2.0 mL) was added and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate-hexane to give the title compound (67 mg) as white crystals.

¹H-NMR (DMSO-d₆) δ: 0.93 (9H, s), 2.77 (2H, d, J=6.0 Hz), 3.49 (4H, s), 3.83 (2H, t, J=4.7 Hz), 4.63-4.72 (3H, m), 5.63 (1H, t, J=6.0 Hz), 6.02-6.05 (1H, m), 6.24 (1H, t, J=3.0 Hz), 6.34-6.36 (1H, m), 6.51 (1H, d, J=3.0 Hz), 7.00 (1H, t, J=6.0 Hz), 7.08 (1H, d, J=9.0 Hz), 7.56-7.60 (1H, m), 7.68 (1H, d, J=3.0 Hz), 7.95 (1H, d, J=3.0 Hz), 8.33 (1H, s), 8.91 (1H, s).

### Example C-40

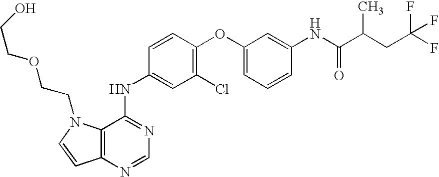

### Production of N-{3-[2-chloro-4-({5-[2-(2-hydroxyethoxy)ethyl]-5H-pyrrolo[3,2-d]pyrimidin-4-yl}amino)phenoxy]phenyl}-4,4,4-trifluoro-2-methylbutanamide

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (50 mg), 4,4,4-trifluoro-2-methylbutanoic acid (47 mg), 1-ethyl-3-(3-dimethylaminopropyl)carbodiimide hydrochloride (125 mg), 1-hydroxybenzotriazole (5 mg), triethylamine (0.7 mL), tetrahydrofuran (7.0 mL), 1N aqueous sodium hydroxide solution (1.5 mL), tetrahydrofuran (3.0 mL) and methanol (2.0 mL) and in the same manner as in Example C-37, the title compound (25 mg) was obtained as crystals.

¹H-NMR (DMSO-d₆) δ: 1.18 (3H, d, J=6.0 Hz), 2.26-2.85 (3H, m), 3.49 (4H, s), 3.84 (2H, t, J=6.0 Hz), 4.66 (2H, t, J=6.0 Hz), 4.71 (1H, m), 6.52 (1H, d, J=3.0 Hz), 6.68-6.70 (1H, m), 7.14-7.38 (4H, m), 7.62-7.70 (2H, m), 7.99 (1H, d, J=3.0 Hz), 8.34 (1H, s), 8.95 (1H, s), 10.14 (1H, s).

### Example C-41

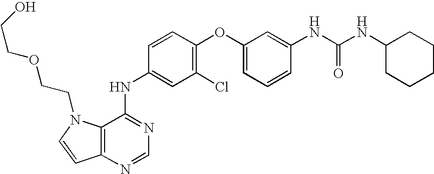

### Production of N-{3-[2-chloro-4-({5-[2-(2-hydroxyethoxy)ethyl]-5H-pyrrolo[3,2-d]pyrimidin-4-yl}amino)phenoxy]phenyl}-N′-cyclohexylurea

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (240 mg), toluene (15 mL), triethylamine (2.0 mL), cyclohexylisocyanate (137 mg), 1N aqueous sodium hydroxide solution (3.0 mL), tetrahydrofuran (6.0 mL) and methanol (6.0 mL) and in the same manner as in Example C-35(ii), the title compound (56 mg) was obtained as crystals.

¹H-NMR (DMSO-d₆) δ: 1.09-1.82 (10H, m), 3.34-3.51 (1H, m), 3.49 (4H, s), 3.84 (2H, t, J=6.0 Hz), 4.64 (2H, t, J=6.0 Hz), 4.71 (1H, m), 6.00 (1H, d, J=9.0 Hz), 6.46-6.52 (2H, m), 6.96-7.21 (4H, m), 7.61-7.69 (2H, m), 7.98 (1H, d, J=2.7 Hz), 8.34 (1H, s), 8.41 (1H, s), 8.93 (1H, s).

### Example C-42

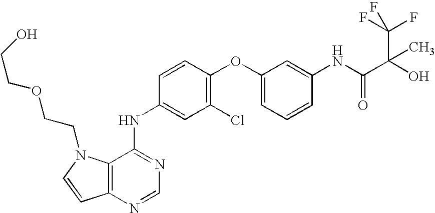

### Production of N-{3-[2-chloro-4-({5-[2-(2-hydroxyethoxy)ethyl]-5H-pyrrolo[3,2-d]pyrimidin-4-yl}amino)phenoxy]phenyl}-3,3,3-trifluoro-2-hydroxy-2-5 methylpropanamide

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (201 mg), 3,3,3-trifluoro-2-hydroxy-2-methylpropanoic acid (175 mg), 1-ethyl-3-(3-dimethylaminopropyl)carbodiimide hydrochloride (415 mg), 1-hydroxybenzotriazole (47 mg), triethylamine (1.8mL), tetrahydrofuran (28 mL), 1N aqueous sodium hydroxide solution (4.0 mL), tetrahydrofuran (6.0 mL) and methanol (6.0 mL) and in the same manner as in Example C-37, the title compound (47 mg) was obtained as crystals.

¹H-NMR (DMSO-d₆) δ: 1.01 (3H, s), 3.49 (4H, s), 3.84 (2H, t, J=6.0 Hz), 4.65 (2H, t, J=6.0 Hz), 4.71 (1H, m), 6.52 (1H, d, J=3.0 Hz), 6.60-6.70 (1H, m), 7.14 (1H, d, J=6.0 Hz), 7.22-7.45 (3H, m), 7.60-7.65 (1H, m), 7.71 (1H, d, J=3.0 Hz), 7.98 (1H, d, J=3.0 Hz), 8.36 (1H, s), 8.98 (1H, s), 9.22 (1H, s).

### Example C-43

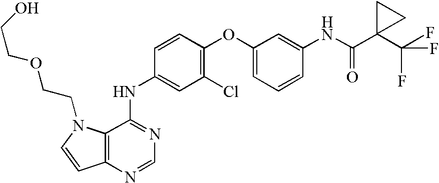

### Production of N-{3-[2-chloro-4-({5-[2-(2-hydroxyethoxy)ethyl]-5H-pyrrolo[3,2-d]pyrimidin-4-yl}amino)phenoxy]phenyl}-1-(trifluoromethyl)cyclopropanecarboxamide

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (220 mg), 1-(trifluoromethyl)cyclopropanecarboxylic acid (300 mg), 1-ethyl-3-(3-dimethylaminopropyl)carbodiimide hydrochloride (450 mg), 1-hydroxybenzotriazole (110 mg), triethylamine (2.6 mL), tetrahydrofuran (15 mL), 1N aqueous sodium hydroxide solution (4.0 mL), tetrahydrofuran (6.0 mL) and methanol (6.0 mL) and in the same manner as in Example C-37, the title compound (23 mg) was obtained as crystals.

¹H-NMR (DMSO-d₆) δ: 1.14-1.31 (2H, m), 1.42-1.45 (2H, m), 3.49 (4H, s), 3.84 (2H, t, J=6.0 Hz), 4.64 (2H, t, J=6.0 Hz), 4.71 (1H, m), 6.51 (1H, d, J=3.0 Hz), 6.70-6.73 (1H, m), 7.15-7.41 (4H, m), 7.60-7.69 (2H, m), 7.99 (1H, d, J=3.0 Hz), 8.34 (1H, s), 8.95 (1H, s), 9.84 (1H, s).

### Example C-44

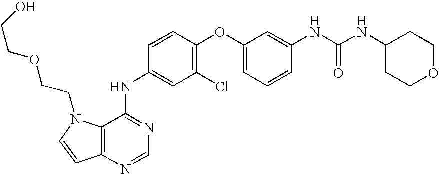

### Production of N-{3-[2-chloro-4-({5-[2-(2-hydroxyethoxy)ethyl]-5H-pyrrolo[3,2-d]pyrimidin-4-yl}amino)phenoxy]phenyl}-N′-(tetrahydro-2H-pyran-4-yl)urea

To a solution of 1,1′-carbonylbis(1H-imidazole) (401 mg) in toluene (10 mL) was added tetrahydro-2H-pyran-4-amine (250 mg) and the mixture was stirred at room temperature for 1 hr. 2-[2-(4-{[4-(3-Aminophenoxy)-3-chlorophenyl]amino}-5H-pyrrolo[3,2-d]pyrimidin-5-yl)ethoxy]ethyl benzoate dihydrochloride (220 mg) and triethylamine (2.0 mL) were added, and the mixture was stirred at 70° C. for 30 min. Water was added to the reaction mixture, and the mixture was extracted with ethyl acetate. The organic layer was washed with saturated brine and dried over anhydrous magnesium sulfate. The solvent was evaporated under reduced pressure and the obtained residue was dissolved in methanol (8.0 mL) and tetrahydrofuran (2.0 mL). 1N Aqueous sodium hydroxide solution (3.0 mL) was added and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The solvent was evaporated under reduced pressure and the obtained residue was crystallized from ethyl acetate-hexane to give the title compound (12 mg) as white crystals.

¹H-NMR (DMSO-₆) δ: 1.09-1.82 (4H, m), 3.20-3.63 (4H, m), 3.33-3.55 (1H, m), 3.49 (4H, s), 3.84 (2H, t, J=6.0, Hz), 4.64 (2H, t; J=6.0 Hz), 4.71 (1H, m), 6.00 (1H, d, J=9.0 Hz), 6.45-6.52 (2H, m), 6.97-7.21 (4H, m), 7.59-7.71 (2H, m), 7.99 (1H, d, J=2.7 Hz), 8.34 (1H, s), 8.41 (1H, s), 8.94 (1H, s).

### Example C-45

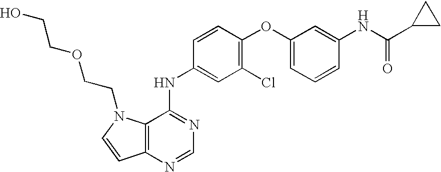

### Production of N-{3-[2-chloro-4-({5-[2-(2-hydroxyethoxy)ethyl]-5H-pyrrolo[3,2-d]pyrimidin-4-yl}amino)phenoxy]phenyl}cyclopropanecarboxamide

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (200 mg), cyclopropanecarboxylic acid (200 mg), 1-ethyl-3-(3-dimethylaminopropyl)carbodiimide hydrochloride (470 mg), 1-hydroxybenzotriazole (42 mg), triethylamine (2.0 mL), tetrahydrofuran (29 mL), 1N aqueous sodium hydroxide solution (3.0 mL), tetrahydrofuran (6.0 mL) and methanol (6.0 mL) and in the same manner as in Example C-37, the title compound (98 mg) was obtained as crystals.

¹H-NMR (DMSO-d₆) δ: 0.76-0.78 (2H, m), 1.04 (2H, d, J=6.0 Hz), 1.71-1.75 (1H, m), 3.49 (4H, s), 3.84 (2H, t, J=6.0 Hz), 4.66 (2H, t, J=6.0 Hz), 4.71 (1H, m), 6.52 (1H, d, J=3.0 Hz), 6.62-6.66 (1H, m), 7.17 (1H, d, J=9.0 Hz), 7.23-7.31 (3H, m), 7.61-7.69 (2H, m), 7.99 (1H, d, J=3.0 Hz), 8.34 (1H, s), 8.94 (1H, s), 10.25 (1H, s).

### Example C-46

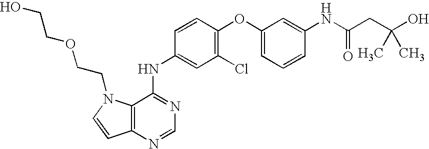

### Production of N-{3-[2-chloro-4-({5-[2-(2-hydroxyethoxy)ethyl]-5H-pyrrolo[3,2-d]pyrimidin-4-yl}amino)phenoxy]phenyl}-3-hydroxy-3-methylbutanamide

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (200. mg), 3-hydroxy-3-methylbutanoic acid (200 mg), 1-ethyl-3-(3-dimethylaminopropyl)carbodiimide hydrochloride (470 mg), 1-hydroxybenzotriazole (41 mg), triethylamine (1.8 mL), tetrahydrofuran (28 mL), 1N aqueous sodium hydroxide solution (2.0 mL), tetrahydrofuran (6.0 mL) and methanol (6.0 mL) and in the same manner as in Example C-37, the title compound (53 mg) was obtained as crystals.

¹H-NMR (DMSO-d₆) δ: 1.21 (6H, s), 2.38 (2H, s), 3.49 (4H, s), 3.84 (2H, t, J=6.0 Hz), 4.64 (2H, t, J=6.0 Hz), 4.71 (1H, s), 4.73 (1H, m), 6.51 (1H, d, J=3.2 Hz), 6.63 (1H, d, J=7.7 Hz), 7.15-7.37 (4H, m), 7.61-7.69 (2H, m), 7.99 (1H, d, J=3.2 Hz), 8.34 (1H, s), 8.94 (1H, s), 9.87 (1H, s).

### Example C-47

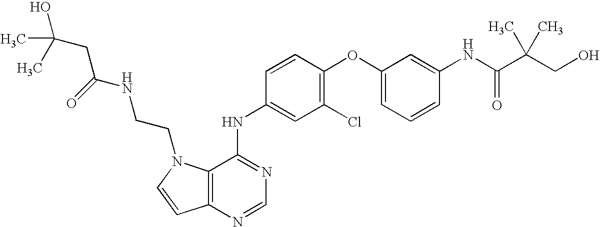

### Production of N-(2-{4-[(3-chloro-4-{3-[(3-hydroxy-2,2-dimethylpropanoyl)amino]phenoxy}phenyl)amino]-5H-pyrrolo[3,2-d]pyrimidin-5-yl}ethyl)-3-hydroxy-3-methylbutanamide

Using 5-(2-aminoethyl)-N-[4-(3-aminophenoxy)-3-chlorophenyl]-5H-pyrrolo[3,2-d]pyrimidin-4-amine trihydrochloride (150 mg), 3-hydroxy-3-methylbutanoic acid (42 mg), 1-ethyl-3-(3-dimethylaminopropyl)carbodiimide hydrochloride (357 mg), 1-hydroxybenzotriazole (18 mg) and triethylamine (0.9 mL) in tetrahydrofuran (4.0 mL) and in the same manner as in Example C-31, the reaction was carried out. Using the obtained crude product and 3-hydroxy-2,2-dimethylpropanoic acid (200 mg), 1-ethyl-3-(3-dimethylaminopropyl)carbodiimide hydrochloride (460 mg), 1-hydroxybenzotriazole (150 mg), triethylamine (2.0 mL) in tetrahydrofuran (7.0 mL) and in the same manner as in Example C-31, the title compound (90 mg) was obtained as crystals.

¹H-NMR (CDCl₃) δ: 1.25 (6H, s), 1.32 (6H, s), 2.48 (2H, s), 2.80 (2H, br s), 3.59 (2H, s), 3.57-3.65 (2H, m), 4.45-4.51 (2H, m), 6.62 (1H, d, J=3.0 Hz), 7.05-7.44(8H, m), 7.73 (1H, dd, J=8.7 Hz, 2.7 Hz), 8.03 (1H, d, J=2.7 Hz), 8.30 (1H, s), 8.50 (1H, s).

### Example C-48

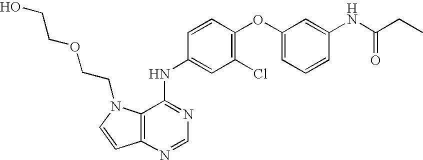

### Production of N-{3-[2-chloro-4-({5-[2-(2-hydroxyethoxy)ethyl]-5H-pyrrolo[3,2-d]pyrimidin-4-yl}amino)phenoxy]phenyl}propanamide

Using 2-[2-(4-{[4-(3-aminophenoxy)-3-chlorophenyl]amino}-5H-pyrrolo[3,2-d]pyrimidin-5-yl)ethoxy]ethyl benzoate dihydrochloride (200 mg), propionic acid (0.5 mL), 1-ethyl-3-(3-dimethylaminopropyl)carbodiimide hydrochloride (560 mg), 1-hydroxybenzotriazole (67 mg), triethylamine (2.1 mL), tetrahydrofuran (10 mL), 1N aqueous sodium hydroxide solution (2.0 mL), tetrahydrofuran (6.0 mL) and methanol (6.0 mL) and in the same manner as in Example C-37, the title compound (70 mg) was obtained as crystals.

¹H-NMR (DMSO-d₆) δ: 1.04 (3H, t, J=7.5 Hz), 2.28 (2H, dd, J=7.5 Hz), 3.49 (4H, s), 3.84 (2H, t, J=6.0 Hz), 4.64 (2H, t, J=6.0 Hz), 4.73 (1H, m), 6.51 (1H, d, J=3.2 Hz), 6.63 (1H, d, J=7.7 Hz), 7.15-7.32 (4H, m), 7.61-7.69 (2H, m), 7.99.(1H, d, J=3.2 Hz), 8.34 (1H, s), 8.94 (1H, s), 9.92 (1H, s).

### Example C-49

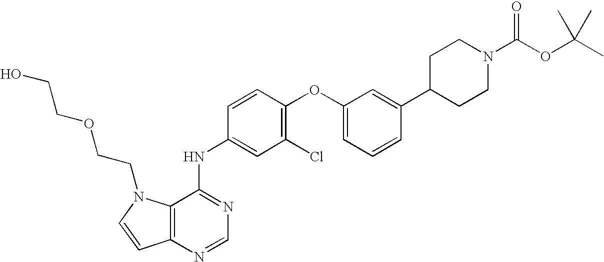

### Production of tert-butyl 4-{3-[2-chloro-4-({5-[2-(2-hydroxyethoxy)ethyl]-5H-pyrrolo[3,2-d]pyrimidin-4-yl}amino)phenoxy]phenyl}piperidine-1-carboxylate

### (i) Production of tert-butyl 4-[3-(2-chloro-4-nitrophenoxy)phenyl]piperidine-1-carboxylate

2-Chloro-1-fluoro-4-nitrobenzene (7.27 g) and tert-butyl 4-(3-hydroxyphenyl)piperidine-1-carboxylate (11.5 g) were dissolved in N,N-dimethylformamide (42 mL), potassium carbonate (8.28 g) was added and the mixture lo was stirred at room temperature for 16 hr. The reaction mixture was diluted with ethyl acetate (300 mL) and washed with water (300 mL). The organic layer was dried over anhydrous magnesium sulfate, and evaporated under reduced pressure. The residue was subjected to silica gel column chromatography (hexane/ethyl acetate=100/0→60/40) to give the title compound (17.6 g) as an oil.

¹H-NMR (CDCl₃) δ: 1.47 (9H, s), 1.50-1.70 (2H, m), 1.84 (2H, d, J=13 Hz), 2.60-2.90 (3H,m), 4.23 (2H, m), 6.87 (1H, d, J=9 Hz), 6.92 (2H, m), 7.12 (1H, d, J=8 Hz), 7.37 (1H, m), 8.04 (1H, dd, J=3 Hz, 9 Hz), 8.38 (1H, d, J=3 Hz).

### (ii) Production of tert-butyl 4-[3-(4-amino-2-chlorophenoxy)phenyl]piperidine-1-carboxylate

tert-Butyl 4-[3-(2-chloro-4-nitrophenoxy)phenyl]piperidine-1-carboxylate (1.9 g) was suspended in ethanol (43 mL)/water (4.81 mL), calcium chloride (270 mg) was added thereto and the mixture was dissolved by heating with stirring at 90° C. for 10 min. Reduced iron (1.63 g) was added thereto, and the mixture was stirred with heating at 90° C. for 16 hr. After cooling to room temperature, the reaction mixture was filtered through celite, and the filtrate was concentrated under reduced pressure. The residual solid was diluted with ethyl acetate (150 mL) and washed with saturated brine (80 mL). The organic layer was dried over anhydrous magnesium sulfate and concentrated under reduced pressure. The residue was purified by silica gel chromatography (hexane/ethyl acetate=80/20→60/40) to give the title compound (1.71 g) as an oil.

¹H-NMR (CDCl₃) δ: 1.48 (9H, s), 1.50-1.70 (2H, m), 1.80 (2H, d, J=13 Hz), 2.58 (1H, m), 2.77 (2H, t, J=13 Hz), 3.69 (2H, br s), 4.22 (2H, m), 6.57 (1H, dd, J=3 Hz, 9 Hz), 6.60-6.90 (5H, m), 7.20 (1H, t, J=8 Hz).

### (iii) Production of tert-butyl 4-{3-[2-chloro-4-({5-[2-(2-hydroxyethoxy)ethyl]-5H-pyrrolo[3,2-d]pyrimidin-4-yl}amino)phenoxy]phenyl}piperidine-1-carboxylate

A mixture of 2-[2-(4-chloro-5H-pyrrolo[3,2-d]pyrimidin-5-yl)ethoxy]ethyl benzoate (150 mg), tert-butyl 4-[3-(4-amino-2-chlorophenoxy)phenyl]piperidine-1-carboxylate (260 mg) and isopropyl alcohol (1.5 mL) was stirred with heating at 80° C. for 16 hr. The reaction mixture was diluted with ethyl acetate (80 mL), and washed with aqueous sodium bicarbonate (40 mL). The organic layer was separated, dried over anhydrous magnesium sulfate, and evaporated under reduced pressure. The residue was purified by silica gel column chromatography (hexane/ethyl acetate=90/10→0/100), and the objective fractions were concentrated under reduced pressure. The obtained, residue was dissolved in methanol (1.89 mL), 1N aqueous sodium hydroxide solution (0.433 mL) was added thereto and the mixture was stirred at room temperature for 2 hr. 1N hydrochloric acid (0.433 mL) was added, and the mixture was diluted with ethyl acetate (30 mL), and washed with saturated brine (30 mL). The organic layer was dried over anhydrous magnesium sulfate and concentrated under reduced pressure, and the residue was subjected to basic silica gel column chromatography (ethyl acetate/methanol=100/0→85/15) to give the title compound (88 mg) as a powder.

¹H-NMR (CDCl₃) δ: 1.47 (9H, s), 1.50-1.80 (2H, m), 1.81 (2H, m), 2.61 (1H, m), 2.77 (2H, m), 3.72 (2H, m), 3.79 (2H, m), 4.01 (2H, t, J=5 Hz), 4.21 (2H, m), 4.55 (2H, t, J=5 Hz), 6.59 (1H, d, J=3 Hz), 6.79 (2H, m), 6.90 (1H, d, J=7.5 Hz), 7.00 (1H, d, J=9 Hz), 7.18-7.30 (2H, m), 7.55 (1H, dd, J=3 Hz, 9 Hz), 7.86 (1H, d, J=3 Hz), 8.49 (1H, s), 8.78 (1H, br s).

### Example C-50

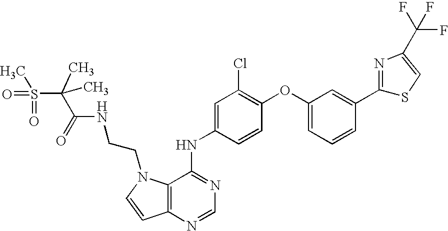

### Production of N-(2-{4-[(3-chloro-4-{3-[4-(trifluoromethyl)-1,3-thiazol-2-yl]phenoxy}phenyl)amino]-5H-pyrrolo[3,2-d]pyrimidin-5-yl}ethyl)-2-methyl-2-(methylsulfonyl)propanamide

Using 5-(2-aminoethyl)-N-(3-chloro-4-{3-[4-(trifluoromethyl)-1,3-thiazol-2-yl]phenoxy}phenyl)-5H-pyrrolo[3,2-d]pyrimidin-4-amine dihydrochloride (200 mg), 2-methyl-2-(methylsulfonyl)propanoic acid (83 mg), 1-hydroxybenzotriazole (75 mg), triethylamine (0.23 mL), 1-ethyl-3-(3-dimethylaminopropyl)carbodiimide hydrochloride (105 mg) and N,N-dimethylformamide (5.0 mL) and in the same manner as in Example C-8(iii), the title compound (171 mg) was obtained as a pale-yellow powder. ¹H-NMR (CDCl₃) δ: 1.70 (6H, s), 2.93 (3H, s), 3.6-3.75 (2H, m), 4.40-4.55 (2H, m), 6.63 (1H, d, J=3.3 Hz), 7.00-7.10 (1H, m), 7.09 (1H, d, J=8.7 Hz), 7.21 (1H, d, J=3.0 Hz), 7.25-7.45 (2H, m), 7.60-7.70 (2H, m), 7.75 (1H, s), 7.80-7.95 (1H, m), 8.04 (1H, d, J=2.4 Hz), 8.36 (1H, s), 8.53 (1H, s).

### Example C-51

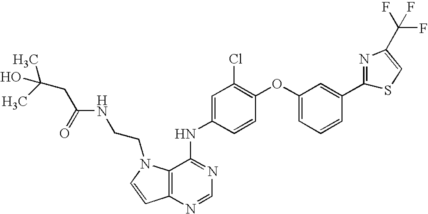

### Production of N-(2-{4-[(3-chloro-4-{3-[4-(trifluoromethyl)-1,3-thiazol-2-yl]phenoxy}phenyl)amino]-5H-pyrrolo[3,2-d]pyrimidin-5-yl}ethyl)-3-hydroxy-3-methylbutanamide

Using 5-(2-aminoethyl)-N-(3-chloro-4-{3-[4-(trifluoromethyl)-1,3-thiazol-2-yl]phenoxy}phenyl)-5H-pyrrolo[3,2-d]pyrimidin-4-amine dihydrochloride (200 mg), 3-hydroxy-3-methylbutanoic acid (59 mg), 1-hydroxybenzotriazole (75 mg), triethylamine (0.23 mL), 1-ethyl-3-(3-dimethylaminopropyl)carbodiimide hydrochloride (105 mg) and N,N-dimethylformamide (5.0 mL) and in the same manner as in Example C-8(iii), the title compound (95.3 mg) was obtained as a white powder.

¹H-NMR (CDCl₃) δ: 1.33 (6H, s), 2.48: (2H, s), 3.60-3.70 (2H, m), 4.40-4.50 (2H, m), 6.60 (1H, d, J=3.0 Hz), 6.85-6.95 (1H, m), 7.00-7.10 (2H, m), 7.18 (1H, d, J=3.0 Hz), 7.40 (1H, t, J=9.0 Hz), 7.60-7.80 (4H, m), 8.07 (1H, s), 8.52 (1H, s), 8.63 (1H, s).

### Example C-52

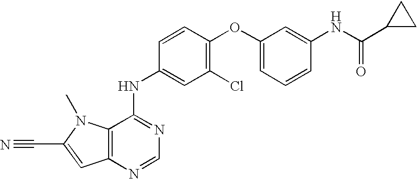

### Production of N-(3-{2-chloro-4-[(6-cyano-5-methyl-5H-pyrrolo[3,2-d]pyrimidin-4-yl)amino]phenoxy}phenyl)cyclopropanecarboxamide

To a solution of diisopropylamine (545 mg) in tetrahydrofuran (15 mL) was added n-butyllithium (2.9 mL) at 0° C. After stirring for 30 min, the mixture was cooled to −78° C., and 4-chloro-5-methyl-5H-pyrrolo[3,2-d]pyrimidine (603 mg) was added thereto. The mixture was stirred for 1 hr, p-toluenesulfonyl cyanide (1300 mg) was added thereto, and the mixture was warmed to −40° C. over 1 hr. Water was added to the reaction mixture and the mixture was extracted with ethyl acetate. The organic layer was washed successively with water and saturated brine, and dried over magnesium sulfate. After concentration under reduced pressure, the residue was separated and purified by basic silica gel column chromatography (eluent, hexane:ethyl acetate=80:20→30:70). After concentration under reduced pressure, resulting crystals were dissolved in isopropyl alcohol (7.0 mL). N-[3-(4-Amino-2-chlorophenoxy)phenyl]cyclopropanecarboxamide (232 mg) was added thereto, and the mixture was stirred at 80° C. for 3 hr. After concentration under reduced pressure, water and saturated aqueous sodium hydrogencarbonate were added and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, hexane:ethyl acetate=80:20→0:100). The objective fractions were concentrated under reduced pressure. The residue was crystallized from ethyl acetate-hexane to give the title compound (103 mg) as a white powder.

¹H-NMR (DMSO-d₆) δ: 0.77(4H, d, J=6.1 Hz), 1.73-1.81 (1H, m), 4.31 (3H, s), 6.66-6.70 (1H, m), 7.21-7.40 (4H, m), 7.53 (1H, s), 7.62 (1H, d, J=8.7 Hz), 7.90 (1H, s), 8.64 (1H, s), 9.87 (1H, br s), 10.25 (1H, s).

### Example C-53

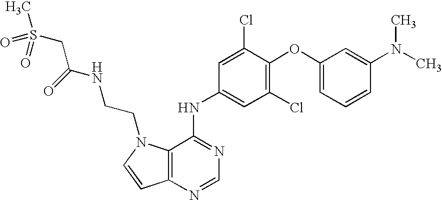

### Production of N-{2-[4-({3,5-dichloro-4-[3-(dimethylamino)phenoxy]phenyl}amino)-5H-pyrrolo[3,2-d]pyrimidin-5-yl]ethyl}-2-(methylsulfonyl)acetamide

### (i) Production of 3,5-dichloro-4-[3-(dimethylamino)phenoxy]aniline

To a solution of 3-(dimethylamino)phenol (470 mg) and 1,3-dichloro-2-iodo-5-nitrobenzene (1.00 g) in N,N-dimethylformamide (15 mL) was added potassium carbonate (850 mg) and the mixture was stirred at room temperature for 18 hr. Under ice-cooling, brine was added to the reaction mixture, and the mixture was extracted twice with ethyl acetate, and the organic layer was dried over anhydrous magnesium sulfate. After concentration under reduced pressure, the residue was separated and purified by silica gel column chromatography (eluent, ethyl acetate:methanol=100:0→95:5) and the obtained crude product was dissolved in 15% water-containing ethanol (23 mL). Reduced iron (750 mg) and calcium chloride (120 mg) were added, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→1:1) to give the title compound (402 mg) as a brown oil.

¹H-NMR (DMSO-d₆) δ: 2.85 (6H, s), 5.58 (2H, s), 5.88 (1H, dd, J=1.9 Hz, 8.0 Hz), 6.19 (1H, t, J=2.2 Hz), 6.37 (1H, dd, J=1.9 Hz, 8.0 Hz), 6.69 (2H, s), 7.04 (1H, t, J=8.3 Hz).

### (ii) Production of N-{2-[4-({3,5-dichloro-4-[3-(dimethylamino)phenoxy]phenyl}amino)-5H-pyrrolo[3,2-d]pyrimidin-5-yl]ethyl}-2-(methylsulfonyl)acetamide

A mixture of tert-butyl[2-(4-chloro-5H-pyrrolo[3,2-d]pyrimidin-5-yl)ethyl]carbamate (150 mg), 3,5-dichloro-4-[3-(dimethylamino)phenoxy]aniline (150 mg) and isopropyl alcohol (8.0 mL) was stirred at 80° C. for 12 hr. Under ice-cooling, to the reaction mixture was added aqueous sodium bicarbonate, and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:hexane=60:40→100:0), the obtained crude product (150 mg) was dissolved in tetrahydrofuran (10 mL), 4N hydrogen chloride/ethyl acetate solution (5.0 mL) was added, and the mixture was stirred at 70° C. for 20 hr. The solvent was evaporated under reduced pressure, ethanol and diisopropyl ether were added to the residue, and precipitated powder was collected by filtration and dissolved in N,N-dimethylformamide (7.0 mL). Methylsulfonylacetic acid (70 mg), 1-ethyl-3-(3-dimethylaminopropyl)carbodiimide hydrochloride (160 mg), 1-hydroxybenzotriazole (70 mg) and triethylamine (0.15 mL) were added to the mixture, and the mixture was stirred at room temperature for 16 hr. Water was added to the reaction mixture and the mixture was extracted lo with ethyl acetate. The organic layer was washed successively with water and saturated brine, and dried over magnesium sulfate. After concentration under reduced pressure, the residue was separated and purified by basic silica gel column chromatography (eluent, ethyl acetate→ethyl acetate:methanol=90:10), and crystallized from diisopropyl ether to give the title compound (74 mg).

¹H-NMR (DMSO-d₆) δ: 2.89 (6H, s), 3.11 (3H, s), 3.44-3.49 (2H, m), 4.06 (2H, s), 4.55-4.59 (2H, m), 5.89-7.11 (5H, m), 7.66-8.69 (5H, m), 8.77 (1H, s).

### Example C-54

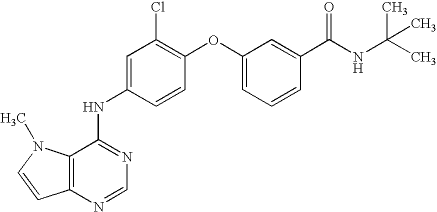

### Production of N-(tert-butyl)-3-{2-chloro-4-[(5-methyl-5H-pyrrolo[3,2-d]pyrimidin-4-yl)amino]phenoxy}benzamide

### (i) Production of methyl 3-{2-chloro-4-[(5-methyl-5H-pyrrolo[3,2-d]pyrimidin-4-yl)amino]phenoxy}benzoate

A mixture of 4-chloro-5-methyl-5H-pyrrolo[3,2-d]pyrimidine (1.01 g), methyl 3-(4-amino-2-chlorophenoxy)benzoate (1.39 g) and isopropyl alcohol (10 mL) was stirred at 80° C. overnight. An aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=60:40→100:0). The objective fraction were concentrated under reduced pressure. Ethyl acetate-diethyl ether was added to the residue, and the precipitated solid was collected by filtration to give the title compound (1.77 g) as a yellow powder.

¹H-NMR (CDCl₃) δ: 3.90 (3H, s), 4.15 (3H, s), 6.56 (1H, d, J=3.3 Hz), 6.83 (1H, br s), 7.06 (1H, d, J=8.8 Hz), 7.16-7.22 (2H, m), 7.40 (1H, t, J=8.0 Hz), 7.46 (1H, dd, J=2.5 Hz, 8.8 Hz), 7.56-7.60 (1H, m), 7.74-7.79 (1H, m), 7.81 (1H, d, J=2.5 Hz), 8.51 (1H, s).

### (ii) Production of 3-{2-chloro-4-[(5-methyl-5H-pyrrolo[3,2-d]pyrimidin-4-yl)amino]phenoxy}benzoic acid

To methyl 3-{2-chloro-4-[(5-methyl-5H-pyrrolo[3,2-d]pyrimidin-4-yl)amino]phenoxy}benzoate (1.68 g) were added methanol (20 mL), tetrahydrofuran (5 mL) and 1N aqueous sodium hydroxide solution (8.2 mL), and the mixture was stirred at room temperature overnight. The reaction mixture was concentrated under reduced pressure, 1N hydrochloric acid (8.2 mL), ethyl acetate and diisopropyl ether were added thereto. The precipitated solid was collected by filtration, washed with water and diisopropyl ether to give the title compound (1.62 g) as a white powder.

¹H-NMR (DMSO-d₆) δ: 4.16 (3H, s), 6.45 (1H, d, J=3.0 Hz), 7.24-7.32 (3H, m), 7.51 (1H, t, J=8.0 Hz), 7.60 (1H, d, J=3.0 Hz), 7.64-7.73 (2H, m), 7.96 (1H, d, J=2.4 Hz), 8.32 (1H, s), 8.62 (1H, br s).

### (iii) Production of N-(tert-butyl)-3-{2-chloro-4-[(5-methyl-5H-pyrrolo[3,2-d]pyrimidin-4-yl)amino]phenoxy}benzamide

A mixture of 3-{2-chloro-4-[(5-methyl-5H-pyrrolo[3,2-d]pyrimidin-4-yl)amino]phenoxy}benzoic acid (197 mg), tert-butylamine (0.105 mL), 1-ethyl-3-(3-dimethylaminopropyl)carbodiimide hydrochloride (115 mg), 1-hydroxybenzotriazole monohydrate (92 mg) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to silica gel column chromatography (eluent, methanol:ethyl acetate=0:100→20:80). The objective fractions were concentrated under reduced pressure. The residue was crystallized from ethyl acetate-diisopropyl ether to give the title compound (215 mg) as a white powder.

¹H-NMR (CDCl₃) δ: 1.45 (9H, s), 4.15 (3H, s), 5.96 (1H, br s), 6.56 (1H, d, J=3.0 Hz), 6.85 (1H, br s), 7.02 (1H, d, J=8.5 Hz), 7.05-7.10 (1H, m), 7.18 (1H, d, J=3.0 Hz), 7.31-7.44 (4H, m), 7.80 (1H, d, J=2.5 Hz), 8.50 (1H, s).

### Example C-55

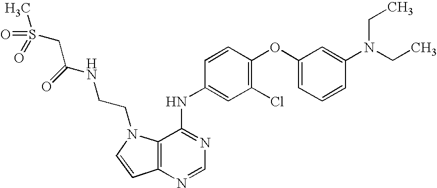

### Production of N-{2-[4-({3-chloro-4-[3-(diethylamino)phenoxy]phenyl}amino)-5H-pyrrolo[3,2-d]pyrimidin-5-yl]ethyl}-2-(methylsulfonyl)acetamide

### (i) Production of 3-(4-amino-2-chlorophenoxy)-N,N-diethylaniline

Using 3-(diethylamino)phenol (920 mg), 3-chloro-4-fluoronitrobenzene (1.01 g), potassium carbonate (1.38 g), N,N-dimethylformamide (20 mL), 5% platinum-activated carbon (300 mg), ethyl acetate (10 mL) and methanol (5.0 mL) and in the same manner as in Example C-6(iv) and (v), the title compound (954 mg) was obtained as crystals.

¹H-NMR (DMSO-d₆) δ: 1.06-1.12 (6H, m), 3.25-3.34 (4H, m), 5.26 (2H, s), 5.94-6.57 (4H, m), 6.68 (1H, d, J=2.0 Hz), 6.86 (1H, d, J=8.0 Hz), 7.12 (1H, t, J=8.3 Hz).

### (ii) Production of N-{2-[4-({3-chloro-4-[3-(diethylamino)phenoxy]phenyl}amino)-5H-pyrrolo[3,2-d]pyrimidin-5-yl]ethyl}-2-(methylsulfonyl)acetamide

Using 3-(4-amino-2-chlorophenoxy)-N,N-diethylaniline (150 mg), tert-butyl[2-(4-chloro-5H-pyrrolo[3,2-d]pyrimidin-5-yl)ethyl]carbamate (150 mg), isopropyl alcohol (10 mL), tetrahydrofuran (15 mL), 4N hydrogen chloride/ethyl acetate solution (5.0 mL), methylsulfonylacetic acid (190 mg), 1-ethyl-3-(3-dimethylaminopropyl)carbodiimide hydrochloride (290 mg), 1-hydroxybenzotriazole (10 mg), triethylamine (4.0 mL) and N,N-dimethylformamide (15 mL) and in the same manner as in Example C-53(ii), the title compound (117 mg) was obtained as crystals.

¹H-NMR (DMSO-d₆) δ: 1.05-1.10 (6H, m), 3.10 (3H, s), 3.27-3.34 (4H, m), 3.44-3.49 (2H, m), 4.05 (2H, s), 4.53-4.57 (2H, m), 6.00-6.49 (4H, m), 7.07-7.91 (5H, m), 8.32 (1H, s), 8.62-8.68 (2H, m).

### Example C-56

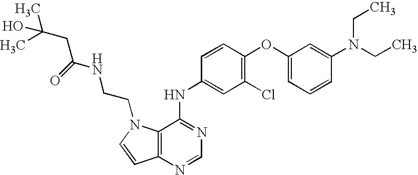

### Production of N-{2-[4-({3-chloro-4-[3-(diethylamino)phenoxy]phenyl}amino)-5H-pyrrolo[3,2-d]pyrimidin-5-yl]ethyl}-3-hydroxy=3-methylbutanamide

Using 3-(4-amino-2-chlorophenoxy)-N,N-diethylaniline (150 mg), tert-butyl[2-(4-chloro-5H-pyrrolo[3,2-d]pyrimidin-5-yl)ethyl]carbamate (150 mg), isopropyl alcohol (10 mL), tetrahydrofuran (15 mL), 4N hydrogen chloride/ethyl acetate solution (5.0 mL), 3-hydroxy-3-methylbutanoic acid (75 mg), 1-ethyl-3-(3-dimethylaminopropyl)carbodiimide hydrochloride (191 mg), 1-hydroxybenzotriazole (5.0 mg), triethylamine (0.3 mL) and N,N-dimethylformamide (6 mL) and in the same manner as in Example C-53(ii), the title compound (94 mg) was obtained as crystals.

¹H-NMR (DMSO-d₆) δ: 1.05-1.17 (12H, m), 2.20 (2H, s), 3.24-3.34 (4H, m), 3.37-3.44 (2H, m), 4.48-4.53 (2H, m), 4.65 (1H, s), 6.01-6.48 (4H, m), 7.06-7.97 (5H, m), 8.22-8.26 (1H, m), 8.31 (1H, s), 8.80 (1H, s).

### Example C-57

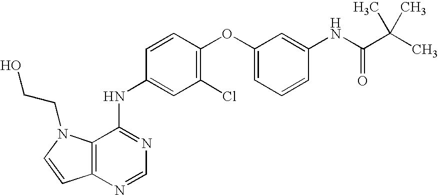

### Production of N-[3-(2-chloro-4-{[5-(2-hydroxyethyl)-5H-pyrrolo[3,2-d]pyrimidin-4-yl]amino}phenoxy)phenyl]-2,2-dimethylpropanamide

A mixture of N-[3-(4-amino-2-chlorophenoxy)phenyl]-2,2-dimethylpropanamide (70 mg) and 2-(4-chloro-5H-pyrrolo[3,2-d]pyrimidin-5-yl)ethyl benzoate (63 mg) was dissolved in isopropyl alcohol (3 mL), pyridine hydrochloride (5 mg) was added thereto, and the mixture was stirred at 80° C. for 16 hr. The reaction mixture was cooled to room temperature, 1N aqueous sodium hydroxide solution (2 mL) was added thereto, and the mixture was stirred at room temperature for 9 hr. A saturated aqueous ammonium chloride solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=10:90→0:100→ethyl acetate:methanol=90:10), and crystallized from diisopropyl ether/ethyl acetate to give the title compound (49 mg) as crystals.

¹H-NMR (DMSO-d₆) δ: 1.16 (9H, s), 3.86-3.89 (2H, m), 4.52-4.55 (2H, m), 6.50-6.66 (2H, m), 7.18-7.97 (7H, m), 8.33 (1H, s), 9.22 (1H, s), 9.83 (1H, br s).

### Example C-58

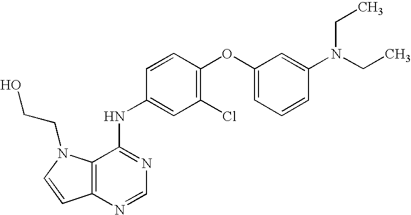

### Production of 2-[4-({3-chloro-4-[3-(diethylamino)phenoxy]phenyl}amino)-5H-pyrrolo[3,2-d]pyrimidin-5-yl]ethanol

Using 3-(4-amino-2-chlorophenoxy)-N,N-diethylaniline (112 mg), 2-(4-chloro-5H-pyrrolo[3,2-d]pyrimidin-5-yl)ethyl benzoate (100 mg), isopropyl alcohol (5.0 mL), 1N aqueous sodium hydroxide solution (5.0 mL) and methanol (10 mL) and in the same manner as in Example C-57, the title compound (52 mg) was obtained as crystals.

¹H-NMR (DMSO-d₆) δ: 1.05-1.09 (6H, m), 3.26-3.33 (4H, m), 3.85-3.88 (2H, m), 4.51-4.54 (2H, m), 5.99-6.41 (3H, m), lo 6.49 (1H, d, J=3.0 Hz), 7.05-7.94 (5H, m), 8.32 (1H, s), 9.76 (1H, br s).

### Example C-59

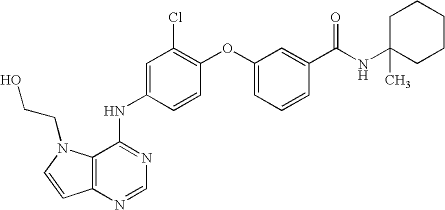

### Production of 3-(2-chloro-4-{[5-(2-hydroxyethyl)-5H-pyrrolo[3,2-d]pyrimidin-4-yl]amino}phenoxy)-N-(1-methylcyclohexyl)benzamide

A mixture of 3-(2-chloro-4-{[5-(2-hydroxyethyl)-5H-pyrrolo[3,2-d]pyrimidin-4-yl]amino}phenoxy)benzoic acid (170 mg), 1-methylcyclohexaneamine hydrochloride (180 mg), 1-ethyl-3-(3-dimethylaminopropyl)carbodiimide hydrochloride (115 mg), 1-hydroxybenzotriazole monohydrate (92 mg), triethylamine (0.170 mL) and N,N-dimethylformamide (5 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to basic silica gel column chromatography (eluent, methanol:ethyl acetate=0:100→20:80) and silica gel column chromatography (eluent, methanol:ethyl acetate=0:100→20:80), and the objective fractions were concentrated under reduced pressure. The residue was crystallized from ethyl acetate-diisopropyl ether to give the title compound (92 mg) as a white powder.

¹H-NMR (CDCl₃) δ: 1.20-1.70 (13H, m), 2.05-2.20 (2H, m), 4.07-4.17 (2H, m), 4.35-4.42 (2H, m), 5.86 (1H, br s), 6.14 (1H, d, J=3.3 Hz), 6.68 (1H, br s), 6.97-7.12 (3H, m), 7.30-7.45 (4H, m), 7.81 (1H, d, J=2.4 Hz), 8.23 (1H, s), 9.69 (1H, br s).

### Example C-60

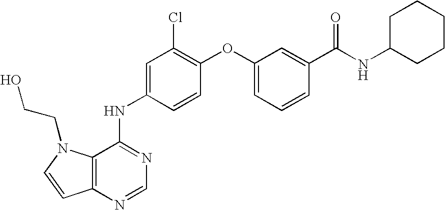

### Production of 3-(2-chloro-4-{[5-(2-hydroxyethyl)-5H-pyrrolo[3,2-d]pyrimidin-4-yl]amino}phenoxy)-N-cyclohexylbenzamide

A mixture of 3-(2-chloro-4-{[5-(2-hydroxyethyl)-5H-pyrrolo[3,2-d]pyrimidin-4-yl]amino}phenoxy)benzoic acid (170 mg), cyclohexanamine (119 mg), 1-ethyl-3-(3-dimethylaminopropyl)carbodiimide hydrochloride (115 mg), 1-hydroxybenzotriazole monohydrate (92 mg) and N,N-dimethylformamide (5 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to silica gel column chromatography (eluent, methanol:ethyl acetate=0:100→20:80). The objective fractions were concentrated under reduced pressure. The residue was crystallized from ethyl acetate-diisopropyl ether to give the title compound (178 mg) as a white powder.

¹H-NMR (CDCl₃) δ: 1.10-1.82 (8H, m), 1.95-2.07 (2H, m), 3.86-4.01 (1H, m), 4.01-4.18 (2H, m), 4.37-4.44 (2H, m), 6.03 (1H, d, J=8.1 Hz), 6.04-6.12 (1H, m), 6.22 (1H, d, J=3.0 Hz), 6.98-7.11 (3H, m), 7.32-7.44 (4H, m), 7.79 (1H, d, J=2.7 Hz), 8.28 (1H, s), 9.57 (1H, br s).

### Example C-61

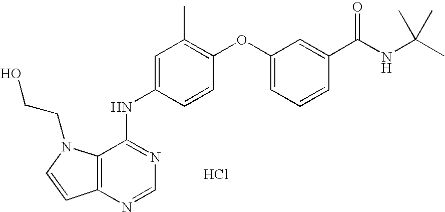

### Production of N-(tert-butyl)-3-(4-{[5-(2-hydroxyethyl)-5H-pyrrolo[3,2-d]pyrimidin-4-yl]amino}-2-methylphenoxy)benzamide hydrochloride

### (i) Production of methyl 3-(2-methyl-4-nitrophenoxy)benzoate

A mixture of methyl 3-hydroxybenzoate (3.04 g), 2-fluoro-5-nitrotoluene (3.10 g), potassium carbonate (4.15 g) and N,N-dimethylformamide (20 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to silica gel column chromatography (eluent, ethyl acetate:hexane=5:95→15:85). The objective fractions were concentrated under reduced pressure to give the title compound (5.66 g) as a pale-yellow solid.

¹H-NMR (CDCl₃) δ: 2.41 (3H, s), 3.91 (3H, s), 6.78 (1H, d, J=8.9 Hz), 7.21-7.27 (1H, m), 7.49 (1H, t, J=7.8 Hz), 7.65-7.68 (1H, m), 7.86-7.91 (1H, m), 8.01 (1H, dd, J=2.8 Hz, 8.9 Hz), 8.17 (1H, d, J=2.8 Hz).

### (ii) Production of 3-(2-methyl-4-nitrophenoxy)benzoic acid

To methyl 3-(2-methyl-4-nitrophenoxy)benzoate (5.66 g) were added isopropyl alcohol (100 mL) and 1N aqueous sodium hydroxide solution (22 mL) and the mixture was stirred at room temperature overnight. To the reaction mixture was added 1N hydrochloric acid (25 mL) and the precipitated solid was collected by filtration, and washed with water to give the title compound (4.54 g) as a white powder.

¹H-NMR (CDCl₃) δ: 2.41 (3H, s), 6.81 (1H, d, J=8.9 Hz), 7.26-7.32 (1H, m), 7.52 (1H, t, J=7.9 Hz), 7.70-7.74 (1H, m), 7.92-7.97 (1H, m), 8.02 (1H,. J=2.9 Hz, 8.9 Hz), 8.17 (1H, d, J=2.9 Hz).

### (iii) Production of N-(tert-butyl)-3-(2-methyl-4-nitrophenoxy)benzamide

A mixture of 3-(2-methyl-4-nitrophenoxy)benzoic acid (820 mg), thionyl chloride (0.438 mL), N,N-dimethylformamide (one drop) and toluene (10 mL) was stirred at 80° C. for 2 hr. Thionyl chloride (0.656 mL) was added and the mixture was further stirred for 1 hr. The reaction mixture was concentrated under reduced pressure, toluene was added, and the mixture was concentrated again under reduced pressure. A solution of the residue in tetrahydrofuran (5 mL) was added to a solution of tert-butylamine (439 mg) and triethylamine (0.627 mL) in tetrahydrofuran (10 mL) and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=10:90→30:70). The objective fractions were concentrated under reduced pressure. The residue was crystallized from ethyl acetate-hexane to give the title compound (948 mg) as a white powder.

¹H-NMR (CDCl₃) δ: 1.47 (9H, s), 2.40 (3H, s), 5.93 (1H, br s), 6.79 (1H, d, J=8.9 Hz), 7.14 (1H, ddd, J=1.2 Hz, 2.5 Hz, 7.8 Hz), 7.40-7.53 (3H, m), 8.00 (1H, dd, J=2.8 Hz, 8.9 Hz), 8.15 (1H, d, J=2.8 Hz).

### (iv) Production of 3-(4-amino-2-methylphenoxy)-N-(tert-butyl)benzamide

To a solution of N-(tert-butyl)-3-(2-methyl-4-nitrophenoxy)benzamide (948 mg) in ethyl acetate (20 mL) was added 5% platinum-activated carbon (50 mg) and the mixture was stirred under a hydrogen atmosphere at room temperature for 6 hr. The catalyst was filtered off, the filtrate was concentrated and the obtained residue was subjected to silica gel column chromatography (eluent, ethyl acetate:hexane=25:75→45:55). The objective fractions were concentrated under reduced pressure to give the title compound (892 mg) as a red purple oil.

¹H-NMR (CDCl₃) δ: 1.45 (9H, s), 2.08 (3H, s), 3.56 (2H, br s), 5.89 (1H, br s), 6.51 (1H, dd, J=2.5 Hz, 8.4 Hz), 6.58 (1H, d, J=2.5 Hz), 6.77 (1H, d, J=8.4 Hz), 6.87-6.94 (1H, m), 7.22-7.30 (3H, m).

### (v) Production of N-(tert-butyl)-3-(4-{[5-(2-hydroxyethyl)-5H-pyrrolo[3,2-d]pyrimidin-4-yl]amino}-2-methylphenoxy)benzamide hydrochloride

A mixture of 2-(4-chloro-5H-pyrrolo[3,2-d]pyrimidin-5-yl)ethyl benzoate (121 mg), 3-(4-amino-2-methylphenoxy)-N-(tert-butyl)benzamide (119 mg) and isopropyl alcohol (5 mL) was stirred at 80° C. overnight. An aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl (acetate:hexane=60:40→100:0). The objective fractions were concentrated under reduced pressure. The residue was crystallized from ethyl acetate-diisopropyl ether, and collected by filtration. To the obtained powder were added methanol (5 mL), tetrahydrofuran (1 mL) and 1N aqueous sodium hydroxide solution (1 mL) and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, methanol:ethyl acetate=0:100→20:80). The objective fractions were concentrated under reduced pressure. The residue was dissolved in ethyl acetate-ethanol and 1N hydrogen chloride/ethyl acetate solution (0.4 mL) was added. The solvent was evaporated under reduced pressure and the obtained residue was crystallized from ethanol-ethyl acetate to give the title compound (139 mg) as a white powder.

¹H-NMR (DMSO-d₆) δ: 1.36 (9H, s), 2.20 (3H, s), 3.90 (2H, t, J=4.4 Hz), 4.69 (2H, t, J=4.4 Hz), 6.30-6.55 (1H, m), 6.69 (1H, d, J=3.0 Hz), 7.02-7.12 (2H, m), 7.28-7.32 (1H, m), 7.43 (1H, t, J=8.0 Hz), 7.48-7.59 (3H, m), 7.81 (1H, br s), 7.99 (1H, d, J=3.0 Hz), 8.72 (1H, s), 10.77 (1H, br s).

### Example C-62

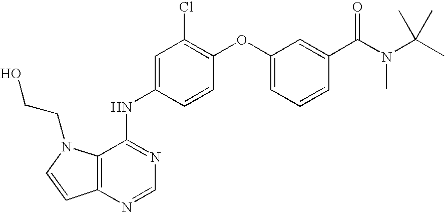

### Production of N-(tert-butyl)-3-(2-chloro-4-{[5-(2-hydroxyethyl)-5H-pyrrolo[3,2-d]pyrimidin-4-yl]amino}phenoxy)-N-methylbenzamide

### (i) Production of methyl 3-(2-chloro-4-nitrophenoxy)benzoate

Using methyl 3-hydroxybenzoate (8.20 g), 2-chloro 1-fluoro-4-nitrobenzene(9.46 g), potassium carbonate (11.2 g) and N,N-dimethylformamide (50 mL) and in the same manner as in Example C-61(i), the title compound (16.0 g) was obtained as a pale-yellow solid.

¹H-NMR (CDCl₃) δ: 3.92 (3H, s), 6.91 (1H, d, J=9.1 Hz), 7.29 (1H, ddd, J=0.8 Hz, 2.6 Hz, 8.0 Hz), 7.52 (1H, t, J=8.0 Hz), 7.70-7.73 (1H, m), 7.91-7.97 (1H, m), 8.07 (1H, dd, J=2.8 Hz, 9.1 Hz), 8.39 (1H, d, J=2.8 Hz).

### (ii) Production of 3-(2-chloro-4-nitrophenoxy)benzoic acid

Using methyl 3-(2-chloro-4-nitrophenoxy)benzoate (7.08 g), isopropyl alcohol (150 mL), tetrahydrofuran (50 mL) and 1N aqueous sodium hydroxide solution (25.3 mL) and in the same manner as in Example C-61(ii), the title compound (5.31 g) was obtained as a white powder.

¹H-NMR (CDCl₃) δ: 6.94 (1H, d, J=9.0 Hz), 7.31-7.37 (1H, m), 7.56 (1H, t, J=8.0 Hz), 7.76-7.79 (1H, m), 7.97-8.03 (1H, m), 8.09 (1H, dd, J=2.6 Hz, 9.0 Hz), 8.40 (1H, d, J=2.6 Hz).

### (iii) Production of N-(tert-butyl)-3-(2-chloro-4-nitrophenoxy)-N-methylbenzamide

Using 3-(2-chloro-4-nitrophenoxy)benzoic acid (881 mg), thionyl chloride (1.09 mL), N,N-dimethylformamide (one drop), toluene (10 mL), tetrahydrofuran (5 mL), N-methyl-tert-butylamine (523 mg), triethylamine (0.627 mL) and tetrahydrofuran (10 mL) and in the same manner as in Example C-61(iii), the title compound (1.14 g) was obtained as a colorless oil.

¹H-NMR (CDCl₃) δ: 1.50 (9H, s), 2.87 (3H, s), 6.92 (1H, d, J=9.1 Hz), 7.08-7.16 (2H, m), 7.30-7.35 (1H, m), 7.45 (1H, t, J=7.8 Hz), 8.06 (1H, dd, J=2.6 Hz, 9.1 Hz), 8.38 (1H, d, J=2.6 Hz).

### (iv) Production of 3-(4-amino-2-chlorophenoxy)-N-(tert-butyl)-N-methylbenzamide

Using N-(tert-butyl)-3-(2-chloro-4-nitrophenoxy)-N-methylbenzamide (1.14 g), ethyl acetate (20 mL) and 5% platinum-activated carbon (50 mg) and in the same manner as in Example C-61(iv), the title compound (868 mg) was obtained as a yellow powder.

¹H-NMR (CDCl₃) δ: 1.48 (9H, s), 2.84 (3H, s), 3.69 (2H, br s), 6.55 (1H, dd, J=2.8 Hz, 8.7 Hz), 6.76 (1H, d, J=2.8 Hz), 6.86-6.93 (3H, m), 7.03-7.08 (1H, m), 7.27 (1H, t, J=7.8 Hz).

### (v) Production of N-(tert-butyl)-3-(2-chloro-4-{[5-(2-hydroxyethyl)-5H-pyrrolo[3,2-d]pyrimidin-4-yl]amino}phenoxy)-N-methylbenzamide

A mixture of 2-(4-chloro-5H-pyrrolo[3,2-d]pyrimidin-5-yl)ethyl benzoate (121 mg), 3-(4-amino-2-chlorophenoxy)-N-(tert-butyl)-N-methylbenzamide (133 mg) and isopropyl alcohol (5 mL) was stirred at 80° C. overnight. An aqueous sodium hydrogencarbonate solution was added to the reaction mixture and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=60:40→100:0). The objective fractions were concentrated under reduced pressure. To the residue were added methanol (5 mL), tetrahydrofuran (1 mL) and 1N aqueous sodium hydroxide solution (1 mL), and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, methanol:ethyl acetate=0:100→20:80). The objective fractions were concentrated under reduced pressure. The residue was crystallized from ethyl acetate-diisopropyl ether to give the title compound (173 mg) as a white powder.

¹H-NMR (CDCl₃) δ: 1.48 (9H, s), 2.86 (3H, s), 4.09 (2H, t, J=4.5 Hz), 4.38 (2H, t, J='4.5 Hz), 6.19 (1H, d, J=3.3 Hz), 6.50-6.90 (1H, m), 6.9.5-7.03 (4H, m), 7.04-7.09 (1H, m), 7.26-7.39 (2H, m), 7.82 (1H, d, J=2.5 Hz), 8.26 (1H, s), 9.73 (1H, br s).

### Example C-63

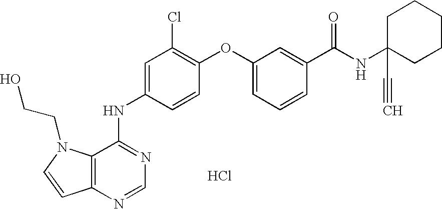

### Production of 3-(2-chloro-4-{[5-(2-hydroxyethyl)-5H-pyrrolo[3,2-d]pyrimidin-4-yl]amino}phenoxy)-N-(1-ethynylcyclohexyl)benzamide hydrochloride

A mixture of 3-(2-chloro-4-{[5-(2-hydroxyethyl)-5H-pyrrolo[3,2-d]pyrimidin-4-yl]amino}phenoxy)benzoic acid (170 mg), 1-ethynylcyclohexaneamine (148 mg), 1-ethyl-3-(3-dimethylaminopropyl)carbodiimide hydrochloride (115 mg), 1-hydroxybenzotriazole monohydrate (92 mg) and N,N-dimethylformamide (5 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to basic silica gel column chromatography (eluent, methanol:ethyl acetate=0:100→20:80). The objective fractions were concentrated under reduced pressure. The residue was dissolved in ethyl acetate-ethanol, and 1N hydrogen chloride/ethyl acetate solution (0.4 mL) was added thereto. The solvent was evaporated under reduced pressure and the obtained residue was crystallized from ethanol-ethyl acetate to give the title compound (160 mg) as a white powder.

¹H-NMR (DMSO-d₆) δ: 1.20-1.37 (1H, m), 1.43-1.64 (5H, m), 1.71-1.88 (2H, m), 2.04-2.18 (2H, m), 3.16 (1H, s), 3.85-3.95 (2H, m), 4.64-4.74 (2H, m), 6.20-6.40 (1H, m), 6.70 (1H, d, J=3.0 Hz), 7.17 (1H, dd, J=2.3 Hz, 8.1 Hz), 7.27-7.35 (2H, m), 7.48 (1H, t, J=8.0 Hz), 7.57-7.65 (2H, m), 7.94 (1H, d, J=2.5 Hz), 7.98-8.03 (1H, m), 8.17 (1H, br s), 8.76 (1H, s), 10.76-10.86 (1H, m).

### Example C-64

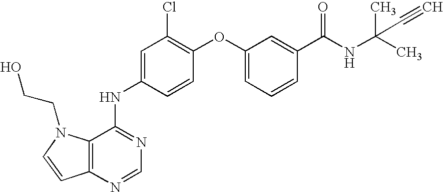

### Production of 3-(2-chloro-4-{[5-(2-hydroxyethyl)-5H-pyrrolo[3,2-d]pyrimidin-4-yl]amino}phenoxy)-N-(1,1-dimethylprop-2-yn-1-yl)benzamide

Using 3-(2-chloro-4-{[5-(2-hydroxyethyl)-5H-pyrrolo[3,2-d]pyrimidin-4-yl]amino}phenoxy)benzoic acid (170 mg), 3-amino-3-methyl-1-butyne (120 mg), 1-ethyl-3-(3-dimethylaminopropyl)carbodiimide hydrochloride (115 mg), 1-hydroxybenzotriazole monohydrate (92 mg) and N,N-dimethylformamide (5 mL) and in the same manner as in Example C-59, the title compound (155 mg) was obtained as a white powder.

¹H-NMR (CDCl₃) δ: 1.75 (6H, s), 2.37 (1H, s), 4.13 (2H, t, J=4.3 Hz), 4.39 (2H, t, J=4.3 Hz), 6.15 (1H, d, J=3.3 Hz), 6.25 (1H, br s), 6.45 (1H, br s), 6.98-7.03 (2H, m), 7.08-7.14 (1H, m), 7.31-7.45 (4H, m), 7.78 (1H, d, J′=2.5 Hz), 8.23 (1H, s), 9.62 (1H, br s).

### Example C-65

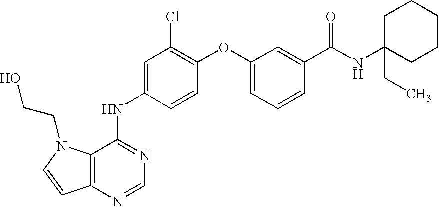

### Production of 3-(2-chloro-4-{[5-(2-hydroxyethyl)-5H-pyrrolo[3,2-d]pyrimidin-4-yl]amino}phenoxy)-N-(1-ethylcyclohexyl)benzamide

Using 3-(2-chloro-4-{[5-(2-hydroxyethyl)-5H-pyrrolo[3,2-d]pyrimidin-4-yl]amino}phenoxy)benzoic acid (170 mg), 1-ethylcyclohexaneamine (153 mg), 1-ethyl-3-(3-dimethylaminopropyl)carbodiimide hydrochloride (115 mg), 1-hydroxybenzotriazole monohydrate (92 mg) and N,N-dimethylformamide (5 mL) and in the same manner as in Example C-59, the title compound (136 mg) was obtained as a white powder.

¹H-NMR (CDCl₃) δ: 0.84 (3H, t, J=7.4 Hz), 1.20-1.70 (8H, m), 1.88 (2H, q, J=7.4 Hz), 2.10-2.21 (2H, m), 4.12 (2H, t, J=4.5 Hz), 4.38 (2H, t, J=4.5 Hz), 5.70 (1H, br s), 6.15 (1H, d, J=3.0 Hz), 6.50 (1H, br s), 6.99 (1H, d, J=3.0 Hz), 7.02 (1H, d, J=8.8 Hz), 7.04-7.11 (1H, m), 7.30-7.44 (4H, m), 7.80 (1H, d, J=2.5 Hz), 8.23 (1H, s), 9.64 (1H, br s).

### Example C-66

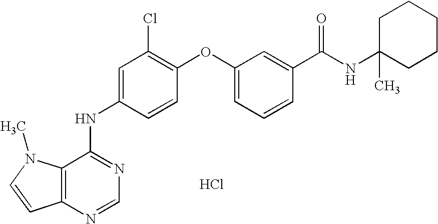

### Production of 3-{2-chloro-4-[(5-methyl-5H-pyrrolo[3,2-d]pyrimidin-4-yl)amino]phenoxy}-N-(1-methylcyclohexyl)benzamide hydrochloride

Using 3-{2-chloro-4-[(5-methyl-5H-pyrrolo[3,2-d]pyrimidin-4-yl)amino]phenoxy}benzoic acid (197 mg), 1-methylcyclohexaneamine hydrochloride (150 mg), 1-ethyl-3-(3-dimethylaminopropyl)carbodiimide hydrochloride (144 mg), 1-hydroxybenzotriazole monohydrate (115 mg), triethylamine (0.139 mL) and N,N-dimethylformamide (5 mL) and in the same manner as in Example C-63, the title compound (178 mg) was obtained as a white powder.

¹H-NMR (DMSO-d₆) δ: 1.18-1.57 (8H, m), 1.31 (3H, s), 2.13-2.29 (2H, m), 4.29 (3H, s), 6.62-6.65 (1H, m), 7.16 (1H, dd, J=2.6 Hz, 8.1 Hz), 7.28 (1H, d, J=8.8 Hz), 7.33 (1H, m), 7.48 (1H, t, J=8.0 Hz), 7.55-7.68 (3H, m), 7.91-7.98 (2H, m), 8.71 (1H, s), 9.78-9.94 (1H, m).

### Example C-67

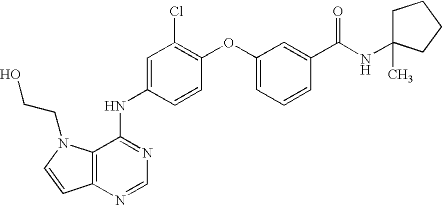

### Production of 3-(2-chloro-4-{[5-(2-hydroxyethyl)-5H-pyrrolo[3,2-d]pyrimidin-4-yl]amino}phenoxy)-N-(1-methylcyclopentyl)benzamide

Using 3-(2-chloro-4-{[5-(2-hydroxyethyl)-5H-pyrrolo[3,2-d]pyrimidin-4-yl]amino}phenoxy)benzoic acid (212 mg), 1-methylcyclopentaneamine hydrochloride (136 mg), 1-ethyl-3-(3-dimethylaminopropyl)carbodiimide hydrochloride (144 mg), 1-hydroxybenzotriazole (101 mg), triethylamine (0.139 mL) and N,N-dimethylformamide (5 mL) and in the same manner as in Example C-59, the title compound (162 mg) was obtained as a white powder.

¹H-NMR (CDCl₃) δ: 1.50 (3H, s), 1.65-1.82 (6H, m), 1.98-2.13 (2H, m), 4.12 (2H, t, J=4.4 Hz), 4.39 (2H, t, J=4.4 Hz), 6.06 (1H, br s), 6.17 (1H, d, J=3.0 Hz), 6.52 (1H, br s), 7.00 (1H, d, J=3.0 Hz), 7.01 (1H, d, J=8.8 Hz), 7.05-7.12 (1H, m), 7.30-7:37 (3H, m), 7.41 (1H, dd, J=2.6 Hz, 8.8 Hz), 7.80 (1H, d, J=2.6 Hz), 8.24 (1H, s), 9.66 (1H, br s).

### Example C-68

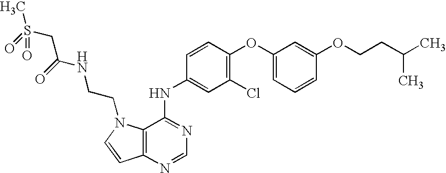

### Production of N-{2-[4-({3-chloro-4-[3-(3-methylbutoxy)phenoxy]phenyl}amino)-5H-pyrrolo[3,2-d]pyrimidin-5-yl]ethyl}-2-(methylsulfonyl)acetamide

### (i) Production of 3-chloro-4-[3-(3-methylbutoxy)phenoxy]aniline

To a solution of 3-(2-chloro-4-nitrophenoxy)phenol (1.0 g) and 1-iodo-3-methylbutane (1.0 mL) in N,N-dimethylformamide (20 mL) was added cesium carbonate (1.6 g) and the mixture was stirred at room temperature for 2 hr. Under ice-cooling, water was added to the reaction mixture, and the mixture was extracted twice with ethyl acetate, and the organic layer was dried over anhydrous magnesium sulfate. After concentration under reduced pressure, the residue was separated and purified by silica gel column chromatography (eluent, hexane:ethyl acetate=1:0→1:1). The obtained crude product was dissolved in 15% water-containing ethanol (25 mL), reduced iron (1.60 g) and calcium chloride (220 mg) were added, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→1:1) to give the title compound (806 mg) as a brown oil.

¹H-NMR (DMSO-d₆) δ: 0.89-0.92 (6H, m), 1.53-1.60 (2H, m), 1.70-1.79 (1H, m), 3.91-3.95 (2H, m), 5.33 (2H, s), 6.31-6.33 (2H, m), 6.53-6.60 (2H, m),6.71-6.72 (1H, m), 6.88-6.91 (1H, m), 7.14-7.20 (1H, m).

### (ii) Production of N-{2-[4-({3-chloro-4-[3-(3-methylbutoxy)phenoxy]phenyl}amino)-5H-pyrrolo[3,2-d]pyrimidin-5-yl]ethyl}-2-(methylsulfonyl)acetamide

Using 3-chloro-4-[3-(3-methylbutoxy)phenoxy]aniline (250 mg), tert-butyl[2-(4-chloro-5H-pyrrolo[3,2-d]pyrimidin-5-yl)ethyl]carbamate (250 mg), isopropyl alcohol (20 mL), ethyl acetate (10 mL), 4N hydrogen chloride/ethyl acetate solution (15 mL), methylsulfonylacetic acid (72 mg), 1-ethyl-3-(3-dimethylaminopropyl)carbodiimide hydrochloride (187 mg), 1-hydroxybenzotriazole (10 mg), triethylamine (1.5 mL) and tetrahydrofuran (10 mL) and in the same manner as in Example C-53(ii), the title compound (277 mg) was obtained as crystals.

¹H-NMR (DMSO-d₆) δ: 0.91-0.93 (6H, m), 1.56-1.78 (3H, m), 3.10 (3H, s), 3.42-3.49 (2H, m), 3.95-4.05 (4H, m), 4.52-4.60 (2H, m), 6.43-6.70 (4H, m), 7.16-7.94 (5H, m), 8.34 (1H, s), 8.64-8.68 (2H, m).

### Example C-69

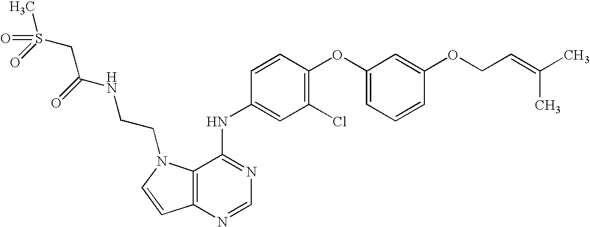

### Production of N-(2-{4-[(3-chloro-4-{3-[(3-methylbut-2-en-1-yl)oxy]phenoxy}phenyl)amino]-5H-pyrrolo[3,2-d]pyrimidin-5-yl}ethyl)-2-(methylsulfonyl)acetamide

### (i) Production of 3-chloro-4-{3-[(3-methylbut-2en-1-yl)oxy]phenoxy}aniline

To a solution of 3-(2-chloro-4-nitrophenoxy)phenol (1.0 g) and 1-bromo-3-methylbut-2-ene (1.5 mL) in N,N-dimethylformamide (20 mL) was added cesium carbonate (1.8 g) and the mixture was stirred at room temperature for 2 hr. Under ice-cooling, water was added to the reaction mixture, and the mixture was extracted twice with ethyl acetate, and the organic layer was dried over anhydrous magnesium sulfate. After concentration under reduced pressure, the residue was separated and purified by silica gel column chromatography (eluent, hexane:ethyl acetate=1:0→41:1). The obtained crude product was dissolved in 15% water-containing ethanol (25 mL), reduced iron (1.60 g) and calcium chloride (220 mg) were added, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→41:1) to give the title compound (730 mg) as a brown oil.

¹H-NMR (DMSO-d₆) δ: 1.62-1.71 (6H, m), 4.42-4.47 (2H, m), 5.32-5.36 (1H, m), 6.32-6.35 (2H, m), 6.56-6.59 (2H, m), 6.71-6.72 (1H, m), 6.87-6.90 (1H, m), 7.13-7.19 (1H, m).

### (ii) Production of N-(2-{4-[(3-chloro-4-{3-[(3-methylbut-2-en-1-yl)oxy]phenoxy}phenyl)amino]-5H-pyrrolo[3,2-d]pyrimidin-5-yl}ethyl)-2-(methylsulfonyl)acetamide

Using 3-chloro-4-{3-[(3-methylbut-2-en-1-yl)oxy]phenoxy}aniline (150 mg), tert-butyl[2-(4-chloro-5H-pyrrolo[3,2-d]pyrimidin-5-yl)ethyl]carbamate (150 mg), isopropyl alcohol (16 mL), ethyl acetate (10 mL), 4N hydrogen chloride/ethyl acetate solution (5.mL), 15 methylsulfonylacetic acid (120 mg), 1-ethyl-3-(3-dimethylaminopropyl)carbodiimide hydrochloride (470 mg), 1-hydroxybenzotriazole (10 mg), triethylamine (1.5 mL) and N,N-dimethylformamide (15 mL) and in the same manner as in Example C-53(ii), the title compound (108 mg) was obtained as crystals.

¹H-NMR (DMSO-d₆) δ: 1.75-1.79 (6H, m), 3.10 (3H, s), 3.40-3.52 (2H, m), 4.03 (2H, s), 4.47-4.60 (4H, m), 5.40-5.51 (1H, m), 6.40-6.70 (4H, m), 7.15-7.95 (5H, m), 8.35 (1H, s), 8.62-8.70 (2H, m).

### Example C-70

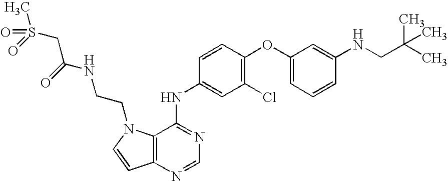

### Production of N-(2-{4-[(3-chloro-4-{3-[(2,2-dimethylpropyl)amino]phenoxy}phenyl)amino]-5H-pyrrolo[3,2-d]pyrimidin-5-yl}ethyl)-2-(methylsulfonyl)acetamide

### (i) Production of 3-(2-chloro-4-nitrophenoxy)-N-(2,2-dimethylpropyl)aniline

3-(2-Chloro-4-nitrophenoxy)aniline hydrochloride (1.98 g) was suspended in tetrahydrofuran (80 mL), and triethylamine (0.87 mL), acetic acid (6.0 mL) and pivalaldehyde (2:10 g) were added thereto, and the mixture was stirred for 30 min. Sodium triacetoxyborohydride (470 mg) was added, and the mixture was further stirred for 2 hr. Water was added to the reaction mixture, and the mixture was extracted with ethyl acetate. The organic layer was washed with saturated brine and dried over anhydrous magnesium sulfate. The solvent was evaporated under reduced pressure and the obtained residue was separated and purified by basic silica gel column chromatography (eluent, hexane :ethyl acetate=95:5→1:1) to give the title compound (1.79 g) as a brown oil.

¹H-NMR (CDCl₃) δ: 0.99 (9H, s), 2.89 (2H, s), 6.31-6.54 (3H, m), 6.89-7.26 (3H, m), 8.01-8.09 (1H, m), 8.37 (1H, s).

### (ii) Production of 3-chloro-4-{3-[(2,2-dimethylpropyl)amino]phenoxy}aniline

3-(2-Chloro-4-nitrophenoxy)-N-(2,2-dimethylpropyl)aniline (1.0 _{g}) was dissolved in 15% water-containing ethanol (30 mL), reduced iron (800 mg) and calcium chloride (100 mg) were added, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→1:1) to give the title compound (737 mg) as a brown oil.

¹H-NMR (DMSO-d₆) δ: 0.95 (9H, s), 2.78-2.82 (2H, m), 6.31-6.54 (3H, m), 6.89-7.26 (3H, m), 8.01-8.09 (1H, m), 8.37 (1H, s).

### (iii) Production of N-(2-{4-[(3-chloro-4-{3-[(2,2-dimethylpropyl)amino]phenoxy}phenyl)amino]-5H-pyrrolo[3,2-d]pyrimidin-5-yl}ethyl)-2-(methylsulfonyl)acetamide

Using 3-chloro-4-{3-[(2,2-dimethylpropyl)amino]phenoxy}aniline (73 mg), tert-butyl [2-(4-chloro-5H-pyrrolo[3,2-d]pyrimidin-5-yl)ethyl]carbamate (71 mg), isopropyl alcohol (5 mL), ethyl acetate (10 mL), 4N hydrogen chloride/ethyl acetate solution (10 mL), methylsulfonylacetic acid (61 mg), 1-ethyl-3-(3-dimethylaminopropyl)carbodiimide hydrochloride (220 mg), 1-hydroxybenzotriazole (5.0 mg), triethylamine (1.5 mL) and N,N-dimethylformamide (7.0 mL) and in the same manner as in Example C-53(ii), the title compound (48 mg) was obtained as crystals.

¹H-NMR (DMSO-d₆) δ: 0.93 (9H, s), 2.76-2.79 (2H, m), 3.10 (3H, s), 3.42-3.48 (2H, m), 4.05 (2H, s), 4.53-4.59 (2H, m), 5.61-6.50 (5H, m), 6.98-7.12 (2H, m), 7.61-7.91 (3H, m), 8.33 (1H, s), 8.64-8.68 (2H, m).

### Example C-71

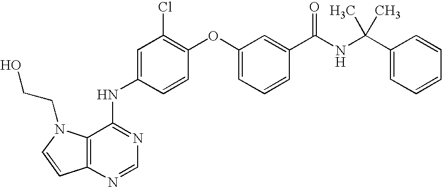

### Production of 3-(2-chloro-4-{[5-(2-hydroxyethyl)-5H-pyrrolo[3,2-d]pyrimidin-4-yl]amino}phenoxy)-N-(1-methyl-1-phenylethyl)benzamide

Using 3-(2-chloro-4-{[5-(2-hydroxyethyl)-5H-pyrrolo[3,2-d]pyrimidin-4-yl]amino}phenoxy)benzoic acid (170 mg), cumylamine (108 mg), 1-ethyl-3-(3-dimethylaminopropyl)carbodiimide hydrochloride (115 mg), 1-hydroxybenzotriazole (81 mg) and N,N-dimethylformamide (5 mL) and in the same manner as in Example C-59, the title compound (139 mg) was obtained as a white powder.

¹H-NMR (CDCl₃) δ: 1.79 (6H, s), 4.02 (2H, t, J=4.5 Hz), 4.32 (2H, t, J=4.5 Hz), 6.16 (1H, d, J=3.0 Hz), 6.50 (1H, br s), 6.95-7.00 (2H, m), 7.07-7.13 (1H, m), 7.17-7.46 (9H, m), 7.77 (1H, d, J=2.5 Hz), 8.22 (1H, s), 9.66 (1H, br s).

### Example C-72

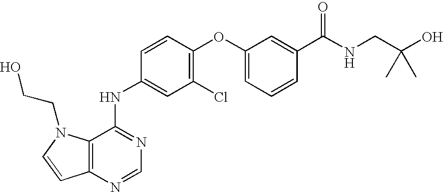

### Production of 3-(2-chloro-4-{[5-(2-hydroxyethyl)-5H-pyrrolo[3,2-d]pyrimidin-4-yl]amino}phenoxy)-N-(2-hydroxy-2-methylpropyl)benzamide

### (i) Production of 3-(2-chloro-4-nitrophenoxy)-N-(2-hydroxy-2-methylpropyl)benzamide

3-(2-Chloro-4-nitrophenoxy)benzoic acid (500 mg) was dissolved in a mixed solvent of tetrahydrofuran (5 mL)/N,N-dimethylformamide (5 mL), and 1-amino-2-methylpropan-2-ol (199 mg), 1-hydroxybenzotriazole (347 mg), triethylamine (0.7 mL) and 1-ethyl-3-(3-dimethylaminopropyl)carbodiimide hydrochloride (496 mg) were added successively, and the mixture was stirred at room temperature for 2.5 hr. Water was added to the reaction mixture, and the mixture was extracted with ethyl acetate. The organic layer was washed, with saturated brine, dried over anhydrous magnesium sulfate and concentrated under reduced pressure. The residue was separated and purified by silica gel column chromatography (hexane:ethyl acetate=33:67→0:100) to give the title compound (448 mg) as a white powder.

¹H-NMR (CDCl₃) δ: 1.30 (6H, s), 3.48 (2H, d, J=5.8 Hz), 6.60 (1H, br s), 6.92 (1H, d, J=9.1 Hz), 7.19-7.25 (1H, m), 7.47-7.58 (2H, m), 7.61-7.69 (1H, m), 8.07 (1H, dd, J=2.8 Hz, 9.1 HZ), 8.39 (1H, d, J=2.8 Hz).

### (ii) Production of 3-(4-amino-2-chlorophenoxy)-N-(2-hydroxy-2-methylpropyl)benzamide

3-(2-Chloro-4-nitrophenoxy)-N-(2-hydroxy-2-methylpropyl)benzamide (446 mg) was dissolved in a mixed solvent of ethanol (13.5 mL)/water (1.5 mL), reduced iron (347 mg) and calcium chloride (68 mg) were added, and the mixture was stirred with heating under reflux for 15 hr. The reaction mixture was filtered through celite, and the filtrate was concentrated under reduced pressure. The residue was dissolved in ethyl acetate, and the solution was washed successively with water and saturated brine, dried over anhydrous magnesium sulfate and concentrated under reduced pressure. The residue was separated and purified by silica gel column chromatography (hexane:ethyl acetate=33:67→0:100) to give the title compound (349 mg) as a colorless oil.

¹H-NMR (CDCl₃) δ: 1.27 (6H, s), 2.38 (1H, br s), 3.44 (2H, d, J=6.0 Hz), 3.71 (2H, br s), 6.53-6.64 (1H, m), 6.57 (1H, dd, J=2.8 Hz, 8.7 Hz), 6.78 (1H, d, J=2.8 Hz), 6.90 (1H, d, J=8.7 Hz), 6.97-7.03 (1H, m), 7.29-7.37 (2H, m), 7.38-7.44 (1H, m).

### (iii) Production of 3-(2-chloro-4-{[5-(2-hydroxyethyl)-5H-pyrrolo[3,2-d]pyrimidin-4-yl]amino}phenoxy)-N-(2-hydroxy-2-methylpropyl)benzamide

A mixture of 2-(4-chloro-5H-pyrrolo[3,2-d]pyrimidin-5-yl)ethyl benzoate (84.3 mg) and 3-(4-amino-2-chlorophenoxy)-N-(2-hydroxy-2-methylpropyl)benzamide (110 mg) was dissolved in isopropyl alcohol (2 mL), a catalytic amount of pyridine hydrochloride was added thereto, and the mixture was stirred at 70° C. for 16 hr. After cooling to room temperature, 1N aqueous sodium hydroxide solution (1 mL) was added thereto, and the mixture was stirred at room temperature for 9 hr. A saturated aqueous ammonium chloride solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=10:90→0:100→ethyl acetate:methanol=90:10), and crystallized from diisopropyl ether/ethyl acetate to give the title compound (99.7 mg) as white crystals.

¹H-NMR (DMSO-d₆) δ: 1.09 (6H, s), 3.23 (2H, d, J=6.0 Hz), 3.87 (2H, t, J=4.5 Hz), 4.47-4.61 (3H, m), 6.30 (1H, br s), 6.51 (1H, d, J=3.0 Hz), 7.05-7.13 (1H, m), 7.24 (1H, d, J=8.9 Hz), 7.36-7.42 (1H, m), 7.46 (1H, t, J=7.9 Hz), 7.56-7.64 (2H, m), 7.66 (1H, d, J=3.0 Hz), 7.98 (1H, d, J=2.6 Hz), 8.31 (1H, t, J=6.0 Hz), 8.34 (1H, s), 9.87 (1H, br s).

### Example C-73

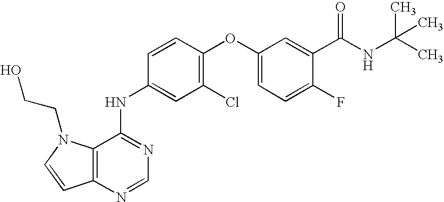

### Production of N-(tert-butyl)-5-(2-chloro-4-{[5-(2-hydroxyethyl)-5H-pyrrolo[3,2-d]pyrimidin-4-yl]amino}phenoxy)-2-fluorobenzamide

Using 5-(4-amino-2-chlorophenoxy)-N-(tert-butyl)-2-fluorobenzamide (260 mg), 2-(4-chloro-5H-pyrrolo[3,2-d]pyrimidin-5-yl)ethyl benzoate (150 mg), isopropyl alcohol (7.0 mL), 1N aqueous sodium hydroxide solution (4.0 mL) and methanol (10 mL) and in the same manner as in Example C-57, the title compound (285 mg) was obtained as crystals.

¹H-NMR (DMSO-d₆) δ: 1.33 (9H, s), 3.85-3.89 (2H, m), 4.51-4.54 (2H, m), 6.50-7.29 (5H, m), 7.58-7.97 (4H, m), 8.33 (1H, s), 9.76(1H, br s).

### Example C-74

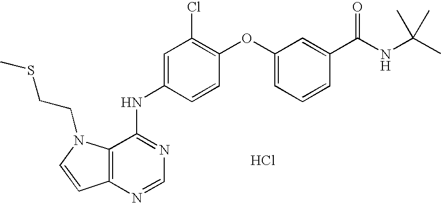

### Production of N-(tert-butyl)-3-[2-chloro-4-({5-[2-(methylthio)ethyl]-5H-pyrrolo[3,2-d]pyrimidin-4-yl}amino)phenoxy]benzamide hydrochloride

### (i) Production of 4-chloro-5-[2-(methylthio)ethyl]-5H-pyrrolo[3,2-d]pyrimidine

A mixture of 4-chloro-5H-pyrrolo[3,2-d]pyrimidine (768 mg), 2-chloroethyl methylsulfide (664 mg), cesium carbonate (1.95 g) and N,N-dimethylformamide (10 mL) was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure. To a mixture of the obtained residue, cesium carbonate (3.91 g) and N,N-dimethylformamide (10 mL) was added dropwise a solution of 2-chloroethyl methylsulfide (553 mg) in N,N-dimethylformamide (3 mL) and the mixture was stirred at room temperature overnight. A solution of 2-chloroethyl methylsulfide (553 mg) in N,N-dimethylformamide (3 mL) was again added dropwise and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=40:60→60:40). The objective fractions were concentrated under reduced pressure to give the title compound (880 mg) as a pale-yellow solid.

¹H-NMR (CDCl₃) δ: 2.04 (3H, s), 2.95 (2H, t, J=6.9 Hz), 4.67 (2H, t, J=6.9 Hz), 6.75 (1H, d, J=3.2 Hz), 7.56 (1H, d, J=3.2 Hz), 8.71 (1H, s).

### (ii) Production of N-(tert-butyl)-3-[2-chloro-4-({5-[2-(methylthio)ethyl]-5H-pyrrolo[3,2-d]pyrimidin-4-yl}amino)phenoxy]benzamide hydrochloride

A mixture of 4-chloro-5-[2-(methylthio)ethyl]-5H-pyrrolo[3,2-d]pyrimidine (455 mg), 3-(4-amino-2-chlorophenoxy)-N-(tert-butyl)benzamide (638 mg) and isopropyl alcohol (10 mL) was stirred at 80° C. overnight. An aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure to give N-(tert-butyl)-3-[2-chloro-4-({5-[2-(methylthio)ethyl]-5H-pyrrolo[3,2-d]pyrimidin-4-yl}amino)phenoxy]benzamide (1.00 g) as an amorphous powder. The obtained N-(tert-butyl)-3-[2-chloro-4-({5-[2-(methylthio)ethyl]-5H-pyrrolo[3,2-d]pyrimidin-4-yl}amino)phenoxy]benzamide (200 mg) was dissolved in ethyl acetate-ethanol, and 1N hydrogen chloride/ethyl acetate solution (0.5 mL) was added. The solvent was evaporated under reduced pressure and the obtained residue was crystallized from ethanol-ethyl acetate to give the title compound (138 mg) as a pale-yellow powder.

¹H-NMR (DMSO-d₆) δ: 1.36 (9H, s), 1.99 (3H, s), 2.88 (2H, t, J=6.4 Hz),4.92 (2H, t, J=6.4 Hz), 6.69 (1H, d, J=3.2 Hz), 7.16 (1H, dd, J=3.0 Hz, 7.7 Hz), 7.24 (1H, d, J=8.8 Hz), 7.36-7.39 (1H, m), 7.47 (1H, t, J=8.0 Hz), 7.57-7.65 (2H, m), 7.84 (1H, br s), 7.92 (1H, d, J=2.5 Hz), 8.06 (1H, d, J=3.2 Hz), 8.73 (1H, s), 10.06 (1H, br s).

### Example C-75

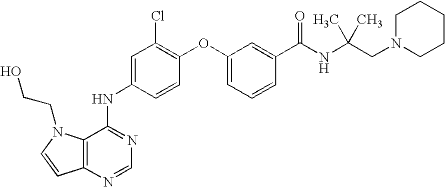

### Production of 3-(2-chloro-4-{[5-(2-hydroxyethyl)-5H-pyrrolo[3,2-d]pyrimidin-4-yl]amino}phenoxy)-N-(1,1-dimethyl-2-(piperidin-1-yl)ethyl)benzamide dihydrochloride

Using 3-(2-chloro-4-{[5-(2-hydroxyethyl)-5H-pyrrolo[3,2-d]pyrimidin-4-yl]amino}phenoxy)benzoic acid (170 mg), 2-methyl-1-(piperidin-1-yl)propan-2-amine (125 mg), 1-ethyl-3-(3-dimethylaminopropyl)carbodiimide hydrochloride (115 mg), 1-hydroxybenzotriazole (81 mg) and N,N-dimethylformamide (5 mL) and in the same manner as in Example C-63, the title compound (136 mg) was obtained as a white powder.

¹H-NMR (DMSO-d₆) δ: 1.33-2.00 (6H, m), 1.51 (6H, s), 2.91-3.09 (2H, m), 3.27-3.62 (4H, m), 3.90 (2H, t, J=4.5 Hz), 4.71 (2H, t, J=4.5 Hz), 6.30-6.65 (1H, m), 6.71 (1H, d, J=3.0 Hz), 7.15 (1H, dd, J=2.3, 7.8 Hz), 7.30 (1H, d, J=8.9 Hz), 7.45-7.53 (2H, m), 7.62 (1H, dd, J=2.5 Hz, 8.9 Hz), 7.73 (1H, d, J=7.7 Hz), 7.96 (1H, d, J=2.5 Hz), 8.02 (1H, d, J=3.0 Hz), 8.17 (1H, br s), 8.76 (1H, s), 9.70 (1H, br s), 10.88 (1H, br s).

### Example C-76

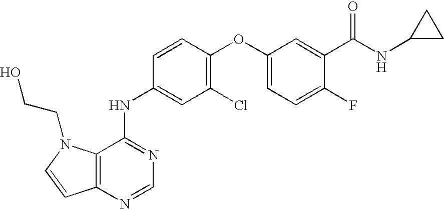

### Production of 5-(2-chloro-4-{[5-(2-hydroxyethyl)-5H-pyrrolo[3,2-d]pyrimidin-4-yl]amino}phenoxy)-N-cyclopropyl-2-fluorobenzamide

### (i) Production of methyl 5-[4-({5-[2-(benzoyloxy)-ethyl]-5H-pyrrolo[3,2-d]pyrimidin-4-yl}amino)-2-chlorophenoxy]-2-fluorobenzoate

Using methyl 5-(4-amino-2-chlorophenoxy)-2-fluorobenzoate (1.50 g), 2-(4-chloro-5H-pyrrolo[3,2-d]pyrimidin-5-yl)ethyl benzoate (1.53 g) and isopropyl alcohol (15 mL) and in the same manner as in Example C-2(v), the title compound (2.12 g) was obtained as crystals.

¹H-NMR (DMSO-d₆) δ: 3.80 (3H, s), 4.54-4.57 (2H, m), 4.94-4.97 (2H, m), 6.56 (1H, s), 7.21-7.79 (12H, m), 8.32 (1H, s), 8.78 (1H, br s).

### (ii) Production of 5-(2-chloro-4-{[5-(2-hydroxyethyl)-5H-pyrrolo[3,2-d]pyrimidin-4-yl]amino}phenoxy)-N-cyclopropyl-2-fluorobenzamide

Using methyl 5-[4-({5-[2-(benzoyloxy)ethyl]-5H-pyrrolo[3,2-d]pyrimidin-4-yl}amino)-2-chlorophenoxy]-2-fluorobenzoate (200 mg), 1N aqueous sodium hydroxide solution (3.0 mL) and tetrahydrofuran (10 mL) and in the same manner as in Example C-2(v), a compound was obtained. The obtained compound was reacted in the same manner as in Example C-9(v) and using cyclopropaneamine (60 mg), 1-ethyl-3-(3-dimethylaminopropyl)carbodiimide hydrochloride (200 mg), 1-hydroxybenzotriazole (5.0 mg), triethylamine (1.5 mL) and N,N-dimethylformamide (5.0 mL) to give the title compound (101 mg) as crystals.

¹H-NMR (DMSO-d₆) δ: 0.52-0.71 (4H, m), 2.77-2.83 (1H, m), 3.86-3.89 (2H, m), 4.52-4.55 (2H, m), 6.15-6.51 (2H, m), 7.01-7.97 (7H, m), 8.33 (1H, s), 8.37-8.38 (1H, m), 9.84(1H, br s).

### Example C-77

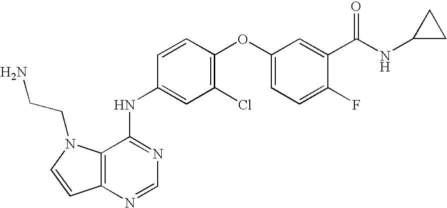

### Production of 5-(4-{[5-(2-aminoethyl)-5H-pyrrolo[3,2-d]pyrimidin-4-yl]amino}-2-chlorophenoxy)-N-cyclopropyl-2-fluorobenzamide

A mixture of tert-butyl[2-(4-chloro-5H-pyrrolo[3,2-d]pyrimidin-5-yl)ethyl]carbamate (286 mg), 5-(4-amino-2-chlorophenoxy)-N-cyclopropyl-2-fluorobenzamide (310 mg) and isopropyl alcohol (5.0 mL) was stirred at 80° C. for 12 hr. Under ice-cooling, aqueous sodium bicarbonate was added to the reaction mixture, and the mixture was extracted with ethyl acetate. The organic layer was washed with brine and dried over anhydrous magnesium sulfate. The residue was separated and purified by silica gel column chromatography (eluent, ethyl acetate:hexane=60:40→100:0). The obtained crude product was dissolved in methanol (10 mL), 4N hydrogen chloride/ethyl acetate solution (5.0 mL) was added, and the mixture was stirred at 70° C. for 20 hr. Ethyl acetate and saturated aqueous sodium hydrogencarbonate were added, and the organic layer was dried over magnesium sulfate. The residue was separated and purified by silica gel column chromatography (hexane:ethyl acetate=10:90→0:100→ethyl acetate:methanol=90:10), and crystallized from diisopropyl ether/ethyl acetate to give the title compound (356 mg) as crystals.

¹H-NMR (DMSO-d₆) δ: 0.53-0.70 (4H, m), 2.77-2.85 (1H, m), 3.06 (2H, br s), 4.36 (2H, br s), 5.95 (2H, br s), 6.48-8.01 (8H, m), 8.31 (1H, s), 8.37-8.38 (1H, m).

### Example C-78

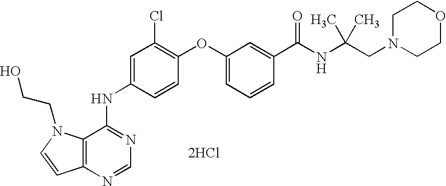

### Production of 3-(2-chloro-4-{[5-(2-hydroxyethyl)-5H-pyrrolo[3,2-d]pyrimidin-4-yl]amino}phenoxy)-N-(1,1-dimethyl-2-(morpholin-4-yl)ethyl)benzamide dihydrochloride

Using 3-(2-chloro-4-{[5-(2-hydroxyethyl)-5H-pyrrolo[3,2-d]pyrimidin-4-yl]amino}phenoxy)benzoic acid (170 mg), 2-methyl-1-(morpholin-4-yl)propan-2-amine (127 mg), 1-ethyl-3-(3-dimethylaminopropyl)carbodiimide hydrochloride (115 mg), 1-hydroxybenzotriazole (81 mg) and N,N-dimethylformamide (5 mL) and in the same manner as in Example C-63, the title compound (128 mg) was obtained as a white powder.

¹H-NMR (DMSO-d₆) δ: 1.52 (6H, s), 3.00-4.20 (12H, m), 4.70 (2H, m), 6.20-6.70 (1H, m), 6.71 (1H, d, J=3.0 Hz), 7.15 (1H, dd, J=2.5 Hz, 7.8 Hz), 7.29 (1H, d, J=8.8 Hz), 7.44-7.54 (2H, m), 7.62 (1H, dd, J=2.5 Hz, 8.8 Hz), 7.6.8-7.76 (1H, m), 7.95 (1H, d, J=2.7 Hz), 8.01 (1H, d, J=2.7 Hz), 8.15 (1H, br s), 8.75 (1H, s), 10.38 (1H, br s), 10.85 (1H, br s).

### Example C-79

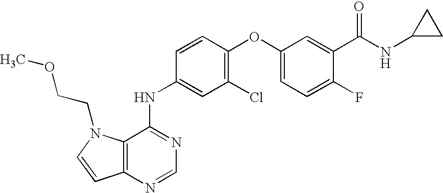

### Production of 5-(2-chloro-4-{[5-(2-methoxyethyl)-5H-pyrrolo[3,2-d]pyrimidin-4-yl]amino}phenoxy)-N-cyclopropyl-2-fluorobenzamide

Using 5-(4-amino-2-chlorophenoxy)-N-cyclopropyl-2-fluorobenzamide (100 mg), 4-chloro-5-(2-methoxyethyl)-5H-pyrrolo[3,2-d]pyrimidine (66 mg) and isopropyl alcohol (5.0 mL) and in the same manner as in Example C-22(i), the title compound (131 mg) was obtained as crystals.

¹H-NMR (DMSO-d₆) δ: 0.52-0.71 (4H, m), 2.77-2.83 (1H, m), 3.31 (3H, s), 3.72-3.75 (2H, m), 4.64-4.67 (2H, m), 6.50-7.95 (8H, m), 8.34 (1H, s), 8.37-8.39 (1H, m), 9.97(1H, br s).

### Example C-80

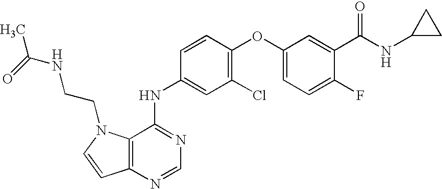

### Production of 5-[4-({5-[2-(acetylamino)ethyl]-5H-pyrrolo[3,2-d]pyrimidin-4-yl}amino)-2-chlorophenoxy]-N-cyclopropyl-2-fluorobenzamide

Using 5-(4-{[5-(2-aminoethyl)-5H-pyrrolo[3,2-d]pyrimidin-4-yl]amino}-2-chlorophenoxy)-N-cyclopropyl-2-fluorobenzamide (100 mg), acetic acid (40 mg), 1-ethyl-3-(3-dimethylaminopropyl)carbodiimide hydrochloride (120 mg), 1-hydroxybenzotriazole (5.0 mg), triethylamine (0.5 mL) and N,N-dimethylformamide (5.0 mL) and in the same manner as in Example C-23(v), the title compound (71 mg) was obtained as crystals.

¹H-NMR (DMSO-d₆) δ: 0.52-0.71 (4H, m), 1.79 (3H, s), 2.79-2.82 (1H, m), 3.33-3.39 (2H, m), 4.48-4.53 (2H, m), 6.49-6.50 (1H, m), 7.02-7.32 (4H, m), 7.63-8.43 (6H, m), 8.79(1H, s).

### Example C-81

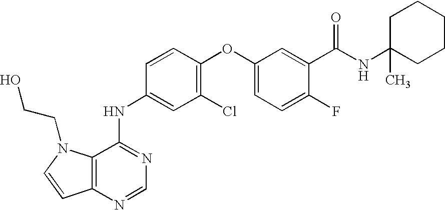

### Production of 5-(2-chloro-4-{[5-(2-hydroxyethyl)-5H-pyrrolo[3,2-d]pyrimidin-4-yl]amino}phenoxy)-2-fluoro-N-(1-methylcyclohexyl)benzamide

Using methyl 5-[4-({5-[2-(benzoyloxy)ethyl]-5H-pyrrolo[3,2-d]pyrimidin-4-yl}amino)-2-chlorophenoxy]-2-fluorobenzoate (150 mg), 1N aqueous sodium hydroxide solution (3.0 mL) and tetrahydrofuran (10 mL) and in the same manner as in Example C-2(v), a compound was obtained. The obtained compound was reacted in the same manner as in Example C-9(v) and using 1-methylcyclohexaneamine (110 mg), 1-ethyl-3-(3-dimethylaminopropyl)carbodiimide hydrochloride (250 mg), 1-hydroxybenzotriazole (5.0 mg), triethylamine (1.5 mL) and N,N-dimethylformamide (5.0 mL) to give the title compound (82 mg) as crystals.

¹H-NMR (DMSO-d₆) δ: 1.20-1.51 (13H, m), 2.14-2.18 (1H, m), 3.86-3.89 (2H, m), 4.52-4.56 (2H, m), 6.49-6.51 (1H, m), 7.17-7.68 (7H, m), 7.98 (1H, s), 8.34(1H, br s), 9.85 (1H, br s).

### Example C-82

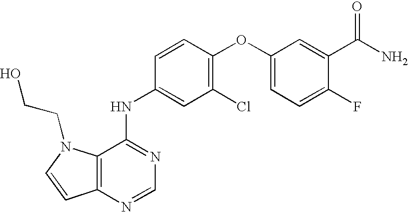

### Production of 5-(2-chloro-4-{[5-(2-hydroxyethyl)-5H-pyrrolo[3,2-d]pyrimidin-4-yl]amino}phenoxy)-2-fluorobenzamide

Using methyl 5-[4-({5-[2-(benzoyloxy)ethyl]-5H-pyrrolo[3,2-d]pyrimidin-4-yl}amino)-2-chlorophenoxy]-2-fluorobenzoate (150 mg), 1N aqueous sodium hydroxide solution (3.0 mL) and tetrahydrofuran (10 mL) and in the same manner as in Example C-2(v), a compound was obtained. The obtained compound was reacted in the same manner as in Example C-9(v) and using 30% ammonia/methanol solution (5.0 mL), 1-ethyl-3-(3-dimethylaminopropyl)carbodiimide hydrochloride (300 mg), 1-hydroxybenzotriazole (30 mg), triethylamine (1.5 mL) and N,N-dimethylformamide (10 mL) to give the title compound (58 mg) as crystals.

¹H-NMR (DMSO-d₆) δ: 3.86-3.89 (2H, m), 4.52-4.56 (2H, m), 6.50-6.51 (1H, m), 7.22-8.04 (9H, m), 8.34(1H, br s), 9.86 (1H, br s).

### Example C-83

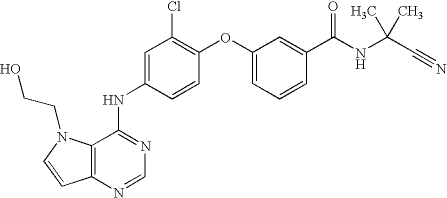

### Production of 3-(2-chloro-4-{[5-(2-hydroxyethyl)-5H-pyrrolo[3,2-d]pyrimidin-4-yl]amino}phenoxy)-N-(1-cyano-1-methylethyl)benzamide

### (i) Production of methyl N-[3-(2-chloro-4-nitrophenoxy)benzoyl]-2-methylalaninate

A mixture of 3-(2-chloro-4-nitrophenoxy)benzoic acid (1.47 g), thionyl chloride (1.00 mL), N,N-dimethylformamide (one drop) and toluene (20 mL) was stirred at 80° C. for 2 hr. After concentration under reduced pressure, toluene was added, and the mixture was again concentrated under reduced pressure. A solution of the residue in tetrahydrofuran (5 mL) was added to a mixture of methyl 2-aminoisobutyrate hydrochloride (922 mg), triethylamine (1.67 mL) and tetrahydrofuran (10 mL) under ice-cooling, and the mixture was stirred under ice-cooling for 1 hr and at room temperature overnight. Water was added to the reaction mixture, and the mixture was extracted with ethyl acetate. The organic layer was washed with saturated brine and,dried over anhydrous magnesium sulfate. The solvent was'evaporated under reduced pressure and the obtained residue was subjected to silica gel column chromatography (eluent, ethyl acetate:hexane=20:80→40:60). The objective fractions were concentrated under reduced pressure to give the title compound (1.72 g) as a white solid.

¹H-NMR (CDCl₃) δ: 1.69 (6H, s), 3.79 (3H, s), 6.84 (1H, br s), 6.92 (1H, d, J=9.2 Hz), 7.20-7.25 (1H, m), 7.48-7.54 (2H, m), 7.61-7.66 (1H, m), 8.08 (1H, dd, J=2.7 Hz, 9.2 Hz), 8.40 (1H, d, J=2.7 Hz).

### (ii) Production of N-[3-(2-chloro-4-nitrophenoxy)benzoyl]-2-methylalanine

To methyl N-[3-(2-chloro-4-nitrophenoxy)benzoyl]-2-methylalaninate (1.72 g) were added isopropyl alcohol (20 mL), tetrahydrofuran (5 mL) and 1N aqueous sodium hydroxide solution (6 mL) and the mixture was stirred at room temperature overnight. 1N hydrochloric acid (6.6 mL) was added to the reaction mixture, and the solvent was evaporated under reduced pressure. Water was added and the precipitated solid was collected by filtration, and washed with water to give the, title compound (1.53 g) as a white powder.

¹H-NMR (DMSO-d₆) δ: 1.45 (6H, s), 7.08 (1H, d, J=9.1 Hz), 7.39 (1H, dd, J=2.5 Hz, 8.0 Hz), 7.61 (1H, t, J=8.0 Hz), 7.68 (1H, m), 7.83 (1H, d, J=8.0 Hz), 8.20 (1H, dd, J=2.7 Hz, 9.1 Hz), 8.50 (1H, d, J=2.7 Hz), 8.55 (1H, br s).

### (iii) Production of 3-(2-chloro-4-nitrophenoxy)-N-(1-cyano-1-methylethyl)benzamide

To a solution of N-[3-(2-chloro-4-nitrophenoxy)benzoyl]-2-methylalanine (1.52 g) in N,N-dimethylformamide (20 mL) were added 1-ethyl-3-(3-dimethylaminopropyl)carbodiimide hydrochloride (920 mg) and 1-hydroxybenzotriazole (649 mg) under ice-cooling, and the mixture was stirred for 1 hr under ice-cooling. 28% aqueous ammonia (1.4 mL) was added to the reaction mixture, and the mixture was stirred under ice-cooling for 1 hr and at room temperature overnight. The reaction mixture was concentrated under reduced pressure, water was added, and the mixture was extracted with ethyl acetate. The organic layer was washed successively with aqueous sodium hydroxide solution and saturated brine, and dried over anhydrous magnesium sulfate. The solvent was evaporated under reduced pressure and the obtained residue was crystallized from ethyl acetate. The obtained powder was subjected to basic silica gel column chromatography (eluent, ethyl acetate). The objective fractions were concentrated under reduced pressure to give N-(2-amino-1,1-dimethyl-2-oxoethyl)-3-(2-chloro-4-nitrophenoxy)benzamide (0.95 g) as a white powder. To a solution of the obtained N-(2-amino-1,1-dimethyl-2-oxoethyl)-3-(2-chloro-4-nitrophenoxy)benzamide (0.95 g) and triethylamine (1.12 mL) in tetrahydrofuran (30 mL) was added dropwise a solution of trifluoroacetic anhydride (0.556 mL) in tetrahydrofuran (5 mL) under ice-cooling, and the mixture was stirred at room temperature overnight. Triethylamine (0.697 mL) and trifluoroacetic anhydride (0.348 mL) were again added to the reaction mixture under ice-cooling, and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=15:85→50:50). The objective fractions were concentrated under reduced pressure to give the title compound (419 mg) as a white amorphous powder.

¹H-NMR (CDCl₃) δ: 1.82 (6H, s), 6.21 (1H, br s), 6.93 (1H, d, J=9.1 Hz), 7.22-7.27 (1H, m), 7.48-7.55 (2H, m), 7.58-7.63 (1H, m), 8.08 (1H, dd, J=2.6, 9.1 Hz), 8.39 (1H, d, J=2.6 Hz).

### (iv) Production of 3-(4-amino-2-chlorophenoxy)-N-(1-cyano-1-methylethyl)benzamide

To a solution of 3-(2-chloro-4-nitrophenoxy)-N-(1-cyano-1-methylethyl)benzamide (419 mg) in ethyl acetate (10 mL) was added 5% platinum-activated carbon (20 mg) under a hydrogen atmosphere and the mixture was stirred at room temperature for 6 hr. The catalyst was filtered off, the filtrate was concentrated and the obtained residue was subjected to silica gel column chromatography (eluent, ethyl acetate:hexane=30:70→60:40). The objective fractions were concentrated under reduced pressure to give the title compound (283 mg) as a yellow-green amorphous powder.

¹H-NMR (CDCl₃) δ: 1.80 (6H, s), 3.72 (2H, br s), 6.15 (1H, br s), 6.58 (1H, dd, J=2.7 Hz, 8.4 Hz), 6.78 (1H, d, J=2.7 Hz), 6.90 (1H, d, J=8.4 Hz), 7.00-7.10 (1H, m), 7.25-7.40 (3H, m).

### (v) Production of 3-(2-chloro-4-{[5-(2-hydroxyethyl)-5H-pyrrolo[3,2-d]pyrimidin-4-yl]amino}phenoxy)-N-(1-cyano-1-methylethyl)benzamide

Using 2-(4-chloro-5H-pyrrolo[3,2-d]pyrimidin-5-yl)ethyl benzoate (121 mg), 3-(4-amino-2-chlorophenoxy)-N-(1-cyano-1-methylethyl)benzamide (132 mg), isopropyl alcohol (5 mL), methanol (5 mL), tetrahydrofuran (1 mL) and 1N aqueous sodium hydroxide solution (0.8 mL) and in the same manner as in Example C-62(v), the title compound (68 mg) was obtained as a white powder.

¹H-NMR (CDCl₃) δ: 1.80 (6H, s), 4.13 (2H, t, J=4.3 Hz), 4.38 (2H, t, J=4.3 Hz), 6.24 (1H, d, J=3.0 Hz), 6.50 (1H, br s), 6.97 (1H, d, J=9.1 Hz), 7.06 (1H, d, J=3.0 Hz), 7.13-7.20 (1H, m), 7.28-7.44 (4H, m), 7.76 (1H, d, J=2.7 Hz), 8.24 (1H, s), 9.58 (1H, br s).

### Example C-84

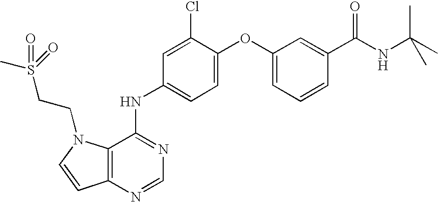

### Production of N-(tert-butyl)-3-[2-chloro-4-({5-[2-(methylsulfonyl)ethyl]-5H-pyrrolo[3,2-d]pyrimidin-4-yl}amino)phenoxy]benzamide

To a solution of N-(tert-butyl)-3-[2-chloro-4-({5-[2-(methylthio)ethyl]-5H-pyrrolo[3,2-d]pyrimidin-4-yl}amino)phenoxy]benzamide (255 mg) in methanol (2 mL) was added a solution of OXONE® monopersulfate compound (615 mg) in water (1 mL) under ice-cooling. Methanol (18 mL) and water (9 mL) were added thereto and the mixture was stirred at room temperature overnight. An aqueous sodium hydrogencarbonate solution was added to the reaction mixture and the mixture was extracted with ethyl acetate. The organic layer was washed successively with 5% aqueous sodium thiosulfate solution and saturated brine, and dried over anhydrous magnesium sulfate. The solvent was evaporated under reduced pressure and the obtained residue was subjected to silica gel column chromatography (eluent, ethyl acetate:hexane=60:40→100:0→methanol:ethyl acetate→10:90). The objective fractions were concentrated under reduced pressure. The residue was crystallized from ethyl acetate-diisopropyl ether to give the title compound (207 mg) as a white powder.

¹H-NMR (CDCl₃) δ: 1.46 (9H, s), 2.73 (3H, s), 3.67 (2H, t, J=6.2 Hz), 4.87 (2H, t, J=6.2 Hz), 5.95 (1H, br s), 6.74 (1H, d, J=3.4 Hz), 7.00-7.12 (2H, m), 7.31-7.40 (4H, m), 7.48 (1H, dd, J=2.7 Hz, 8.7 Hz), 7.86 (1H, d, J=2.7 Hz), 7.97 (1H, br s), 8.56 (1H, s).

### Example C-85

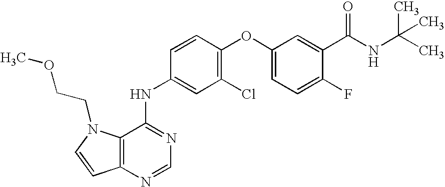

### Production of N-(tert-butyl)-5-(2-chloro-4-{[5-(2-methoxyethyl)-5H-pyrrolo[3,2-d]pyrimidin-4-yl]amino}phenoxy)-2-fluorobenzamide

Using 5-(4-amino-2-chlorophenoxy)-N-(tert-butyl)-2-fluorobenzamide (60 mg), 4-chloro-5-(2-methoxyethyl)-5H-pyrrolo[3,2-d]pyrimidine (38 mg) and isopropyl alcohol (3.0 mL) and in the same manner as in Example C-22(i), the title compound (84 mg) was obtained as crystals.

¹H-NMR (DMSO-d₆) δ: 1.32 (9H, s), 3.72-3.76 (2H, m), 3.80 (3H, s), 4.64-4.68 (2H, m), 6.51-7.99 (8H, m), 8.36 (1H, s), 9.01 (1H, s), 9.97(1H, br s).

### Example C-86

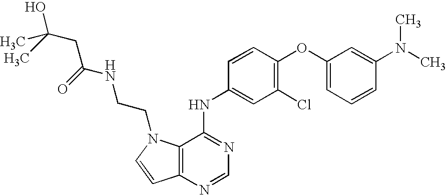

### Production of N-{2-[4-({3-chloro-4-[3-(dimethylamino)phenoxy]phenyl}amino)-5H-pyrrolo[3,2-d]pyrimidin-5-yl]ethyl}-3-hydroxy-3-methylbutanamide

### (i) Production of 3-(4-amino-2-chlorophenoxy)-N,N-dimethylaniline

Using 3-(dimethylamino)phenol (5.0 g), 3-chloro-4-fluoronitrobenzene (6.38 g), potassium carbonate (5.38 g), N,N-dimethylformamide (100 mL), 5% platinum-activated carbon (0.73 g) and ethyl acetate (75 mL) and in the same manner as in Example C-1(i) and (ii), the title compound (8.95 g) was obtained.

¹H-NMR (DMSO-d₆) δ: 2.84 (6H, s), 5.27 (2H, s), 5.95-6.55 (4H, m), 6.69 (1H, d, J=2.0 Hz), 6.86 (1H, d, J=8.0 Hz), 7.04 (1H, t, J=8.3 Hz).

### (ii) Production of N-{2-[4-({3-chloro-4-[3-(dimethylamino)phenoxy]phenyl}amino)-5H-pyrrolo[3,2-d]pyrimidin-5-yl]ethyl}-3-hydroxy-3-methylbutanamide

Using 3-(4-amino-2-chlorophenoxy)-N,N-dimethylaniline (100 mg), tert-butyl[2-(4-chloro-5H-pyrrolo[3,2-d]pyrimidin-5-yl)ethyl]carbamate (89 mg), isopropyl alcohol (5.0 mL), ethyl acetate (5.0 mL), 4N hydrogen chloride/ethyl acetate solution (5.0 mL), 3-hydroxy-3-methylbutanoic acid (54 mg), 1-ethyl-3-(3-dimethylaminopropyl)carbodiimide hydrochloride (136 mg), 1-hydroxybenzotriazole (5.0 mg), triethylamine (1.0 mL) and tetrahydrofuran (10 mL) and in the same manner as in Example C-53(ii), the title compound (67 mg) was obtained as crystals.

¹H-NMR (DMSO-d₆) δ: 1.13 (6H, s), 2.20 (2H, s), 2.89 (6H, s), 3.38-3.44 (2H, m), 4.49-4.53 (2H, m), 4.66 (1H, s), 6.09-7.15 (6H, m), 7.62-8.26 (4H, m), 8.31 (1H, s), 8.81 (1H, s).

### Example C-87

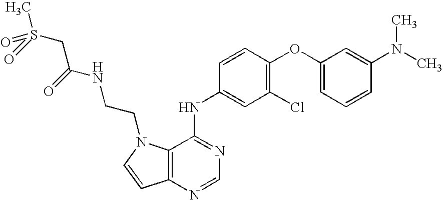

### Production of N-{2-[4-({3-chloro-4-[3-(dimethylamino)phenoxy]phenyl}amino)-5H-pyrrolo[3,2-d]pyrimidin-5-yl]ethyl}-2-(methylsulfonyl)acetamide

Using 3-(4-amino-2-chlorophenoxy)-N,N-dimethylaniline (100 mg), tert-butyl[2-(4-chloro-5H-pyrrolo[3,2-d]pyrimidin-5-yl)ethyl]carbamate (89 mg), isopropyl alcohol (5.0 mL), ethyl acetate (5.0 mL), 4N hydrogen chloride/ethyl acetate solution (5.0 mL), methylsulfonylacetic acid (52 mg), 1-ethyl-3-(3-dimethylaminopropyl)carbodiimide hydrochloride (141 mg), 1-hydroxybenzotriazole (5.0 mg), triethylamine (1.0 mL) and tetrahydrofuran (10 mL) and in the same manner as in Example C-53(ii), the title compound (74 mg) was obtained as crystals.

¹H-NMR (DMSO-d₆) δ: 2.89 (6H, s), 3.09 (3H, s), 3.44-3.47 (2H, m), 4.04 (2H, s), 4.51-4.59 (2H, m), 6.08-7.16 (6H, m), 7.60-7.91 (3H, m), 8.32 (1H, s), 8.61-8.69 (2H, m).

### Example C-88

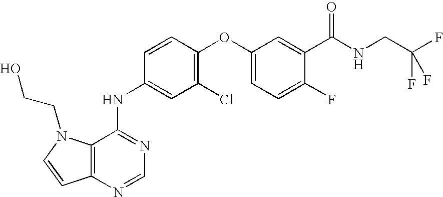

### Production of 5-(2-chloro-4-{[5-(2-hydroxyethyl)-5H-pyrrolo[3,2-d]pyrimidin-4-yl]amino}phenoxy)-2-fluoro-N-(2,2,2-trifluoroethyl)benzamide

Using methyl 5-[4-({5-[2-(benzoyloxy)ethyl]-5H-pyrrolo[3,2-d]pyrimidin-4-yl}amino)-2-chlorophenoxy]-2-fluorobenzoate (100 mg), 1N aqueous sodium hydroxide solution (2.0 mL) and tetrahydrofuran (5.0 mL) and in the same manner as in Example C-2(v), a compound was obtained. The obtained compound was reacted in the same manner as in Example C-9(v) and using 2,2,2-trifluoroethaneamine (70 mg), 1-ethyl-3-(3-dimethylaminopropyl)carbodiimide hydrochloride (220 mg), 1-hydroxybenzotriazole (5.0 mg), triethylamine (0.5 mL) and N,N-dimethylformamide (5.0 mL) to give the title compound (52 mg) as crystals.

¹H-NMR (DMSO-d₆) δ: 3.86-3.90 (2H, m), 3.99-4.10 (2H, m), 4.52-4.56 (2H, m), 6.15-6.52 (1H, m), 7.24-7.99 (7H, m), 8.34 (1H, s), 9.13-9.17 (1H, m), 9.87(1H, br s).

### Example C-89

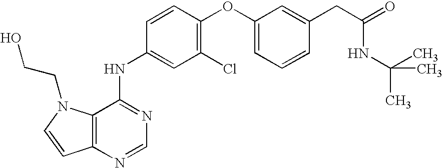

### Production of N-(tert-butyl)-2-[3-(2-chloro-4-{[5-(2-hydroxyethyl)-5H-pyrrolo[3,2-d]pyrimidin-4-yl]amino}phenoxy)phenyl]acetamide

### (i) Production of benzyl[3-(2-chloro-4-nitrophenoxy)phenyl]acetate

Using benzyl (3-hydroxyphenyl)acetate (2.50 g), chloro-4-fluoronitrobenzene (1.83 g), potassium carbonate (1.90 g) and N,N-dimethylformamide (20 mL) and in the same manner as in Example C-1(i), the title compound (1.21 g) was obtained as a brown oil.

¹H-NMR (DMSO-d₆) δ: 3.81(2H, s), 5.12 (2H, s), 7.00-7.25 (4H, m), 7.31-7.37 (5H, m), 7.43-7.48 (1H, m), 8.14-8.18 (1H, m), 8.46-8.47 (1H, m).

### (ii) Production of 2-[3-(4-amino-2-chlorophenoxy)phenyl]-N-(tert-butyl)acetamide

To benzyl[3-(2-chloro-4-nitrophenoxy)phenyl]acetate (1.00 g) were added 1N aqueous sodium hydroxide solution (5.3 mL) and tetrahydrofuran (4 mL) and the mixture was stirred at room temperature for 21 hr. The reaction mixture was neutralized with 1N hydrochloric acid, and aqueous sodium bicarbonate and brine were added. The mixture was extracted with ethyl acetate, and the organic layer was dried over anhydrous magnesium sulfate and concentrated under reduced pressure. The obtained residue, 2-methylpropan-2-amine (1.1 mL), 1-ethyl-3-(3-dimethylaminopropyl)carbodiimide hydrochloride (2.11 g), 1-hydroxybenzotriazole (30 mg), triethylamine (3.5 mL) and N,N-dimethylformamide (10 mL) were reacted in the same manner as in Example C-9(v), and the obtained compound, 5% platinum-activated carbon (130 mg) and ethyl acetate (10 mL) were reacted in the same manner as in Example C-1(ii) to give the title compound (340 mg) as a pale-yellow oil.

¹H NMR (DMSO-d₆) δ: 1.21 (9H, s), 3.61(2H, s), 5.27 (2H, s), 5.92-6.59 (4H, m), 6.65-6.69 (1H, m), 6.87 (1H, d, J=8.0 Hz), 7.13 (1H, t, J=8.3 Hz).

### (iii) Production of N-(tert-butyl)-2-[3-(2-chloro-4-{[5-(2-hydroxyethyl)-5H-pyrrolo[3,2-d]pyrimidin-4-yl]amino}phenoxy)phenyl]acetamide

Using 2-[3-(4-amino-2-chlorophenoxy)phenyl]-N-(tert-butyl)acetamide (330 mg), 2-(4-chloro-5H-pyrrolo[3,2-d]pyrimidin-5-yl)ethyl benzoate (270 mg), isopropyl alcohol (20 mL), 1N aqueous sodium hydroxide solution (2.0 mL) and methanol (6.0 mL) and in the same manner as in Example C-57, the title compound (115 mg) was obtained as crystals.

¹H-NMR (DMSO-d₆) δ: 1.22 (9H, s), 3.34-3.40 (2H, m), 3.84-3.92 (2H, m), 4.51-4.57 (2H, m), 6.30 (1H, br s), 6.51-7.30 (6H, m), 7.56-7.96 (4H, m), 8.36 (1H, s), 9.91(1H, br s).

### Example C-90

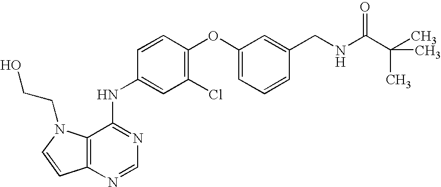

### Production of N-[3-(2-chloro-4-{[5-(2-hydroxyethyl)-5H-pyrrolo[3,2-d]pyrimidin-4-yl]amino}phenoxy)benzyl]-2,2-dimethylpropanamide

### (i) Production of N-[3-(4-amino-2-chlorophenoxy)benzyl]-2,2-dimethylpropanamide

Using N-(3-hydroxybenzyl)-2,2-dimethylpropanamide (2.07 g), 3-chloro-4-fluoronitrobenzene (1.79 g), N,N-dimethylformamide (40 mL), potassium carbonate (1.78 g), 15% water-containing ethanol (23 mL), reduced iron (750 mg) and calcium chloride (120 mg) and in the same manner as in Example C-53(i), the title compound (1.73 g) was obtained as a brown oil.

¹H-NMR (DMSO-d₆) δ: 1.09 (9H, s), 4.21-4.25 (2H, m), 5.26 (2H, s), 5.90-6.58 (4H, m), 6.64-6.68 (1H, m), 6.85-6.98 (1H, m), 7.13 (1H, t, J=8.3 Hz).

### (ii) Production of N-[3-(2-chloro-4-{[5-(2-hydroxyethyl)-5H-pyrrolo[3,2-d]pyrimidin-4-yl]amino}phenoxy)benzyl]-2,2-dimethylpropanamide

Using N-[3-(4-amino-2-chlorophenoxy)benzyl]-2,2-dimethylpropanamide (190 mg), 2-(4-chloro-5H-pyrrolo[3,2-d]pyrimidin-5-yl)ethyl benzoate (172 mg), isopropyl alcohol (5.0 mL), 1N aqueous sodium hydroxide solution (2.0 mL) and methanol (5.0 mL) and in the same manner as in Example C-57, the title compound (121 mg) was obtained as crystals.

¹H-NMR (DMSO-d₆) δ: 1.07 (9H, s), 3.86-3.89 (2H, m), 4.21-4.23 (2H, m), 4.51-4.55 (2H, m), 6.51-7.32 (6H, m), 7.56-8.06 (4H, m), 8.33 (1H, s), 9.82(1H, br s).

### Example C-91

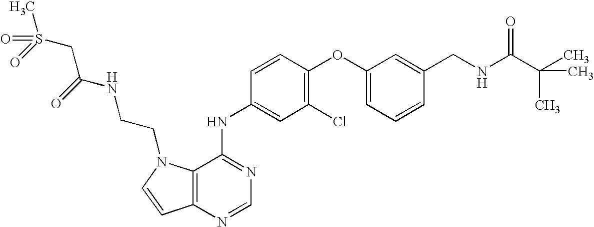

### Production of N-[3-(2-chloro-4-{[5-(2-{[(methylsulfonyl)acetyl]amino}ethyl)-5H-pyrrolo[3,2-d]pyrimidin-4-yl]amino}phenoxy)benzyl]-2,2-dimethylpropanamide

Using N-[3-(4-amino-2-chlorophenoxy)benzyl]-2,2-dimethylpropanamide (350 mg), tert-butyl[2-(4-chloro-5H-pyrrolo[3,2-d]pyrimidin-5-yl)ethyl]carbamate (312 mg), isopropyl alcohol (11 mL), tetrahydrofuran (17 mL), 4N hydrogen chloride/ethyl acetate solution (7.0 mL), methylsulfonylacetic acid (220 mg), 1-ethyl-3-(3-dimethylaminopropyl)carbodiimide hydrochloride (380 mg), 1-hydroxybenzotriazole (20 mg), triethylamine (4.0 mL) and N,N-dimethylformamide (15 mL) and in the same manner as in Example C-53(ii), the title compound (263 mg) was obtained as crystals.

¹H-NMR (DMSO-d₆) δ: 1.08 (9H, s), 3.10 (3H, s), 3.45-3.49 (2H, m), 4.04 (2H, s), 4.22-4.24 (2H, m), 4.54-4.59 (2H, m), 6.48-7.33 (6H, m), 7.62-8.08 (4H, m), 8.34 (1H, s), 8.67(1H, br s).

### Example C-92

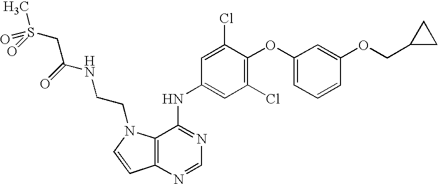

### Production of N-{2-[4-({3,5-dichloro-4-[3-(cyclopropylmethoxy)phenoxy]phenyl}amino)-5H-pyrrolo[3,2-d]pyrimidin-5-yl]ethyl}-2-(methylsulfonyl)acetamide

### (i) Production of 3-(2,6-dichloro-4-nitrophenoxy)phenyl benzoate

To a solution of 3-hydroxyphenyl benzoate (675 mg) and 1,3-dichloro-2-iodo-5-nitrobenzene (1.0 g) in N,N-dimethylformamide (15 mL) was added potassium carbonate (1.25 g) and the mixture was stirred at room temperature for 18 hr. Under ice-cooling, to the reaction mixture was added brine, and the mixture was extracted twice with ethyl acetate, and the organic layer was dried over anhydrous magnesium sulfate. After concentration under reduced pressure, the residue was separated and purified by silica gel column chromatography (eluent, hexane:ethyl acetate=4:1→1:1) to give the title compound (269 mg) as a brown oil.

¹H-NMR (DMSO-d₆) δ: 6.90-6.97 (2H, m), 7.07-7.10 (1H, m), 7.44-7.77 (4H, m), 8.10-8.12 (2H, m), 8.55(2H, s).

### (ii) Production of 3,5-dichloro-4-[3-(cyclopropylmethoxy)phenoxy]aniline

To 3-(2,6-dichloro-4-nitrophenoxy)phenyl benzoate (269 mg) were added methanol (5 mL), tetrahydrofuran (1 mL) and 1N aqueous sodium hydroxide solution (0.6 mL) and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure. To a solution of the obtained residue and 1-(bromomethyl)cyclopropane (0.78 mL) in N,N-dimethylformamide (15 mL) was added potassium carbonate (430 mg) and the mixture was stirred at room temperature for 18 hr. Under ice-cooling, brine was added to the reaction mixture, and the mixture was extracted twice with ethyl acetate, and the organic layer was dried over anhydrous magnesium sulfate. After concentration under reduced pressure, the residue was separated and purified by silica gel column chromatography (eluent, hexane:ethyl acetate=4:1→1:1). The obtained crude product was dissolved in 15% water-containing ethanol (10 mL), reduced iron (250 mg) and calcium chloride (70 mg) were added, and the mixture was stirred at 80° C. for 8 hr. The solid was, removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→0:1) to give the title compound (112 mg) as a brown oil.

¹H-NMR (DMSO-d₆) δ: 0.30-0.57 (4H, m), 1.11-1.35 (1H, m), 3.79-3.81 (2H, m), 5.56 (2H, s), 5.90 (1H, dd, J=2.0 Hz, 8.0 Hz), 6.22 (1H, t, J=2.2 Hz), 6.36 (1H, dd, J=2.0 Hz, 8.0 Hz), 6.71 (2H, s), 7.05 (1H, J=8.3 Hz).

### (iii) Production of N-{2-[4-({3,5-dichloro-4-[3-(cyclopropylmethoxy)phenoxy]phenyl}amino)-5H-pyrrolo[3,2-d]pyrimidin-5-yl]ethyl}-2-(methylsulfonyl)acetamide

Using 3,5-dichloro-4-[3-(cyclopropylmethoxy)phenoxy]aniline (110 mg), tert-butyl [2-(4-chloro-5H-pyrrolo[3,2-d]pyrimidin-5-yl)ethyl]carbamate (101 mg), isopropyl alcohol (5.0 mL), methanol (10 mL), 4N hydrogen chloride/ethyl acetate solution (3.0 mL), methylsulfonylacetic acid (90 mg), 1-ethyl-3-(3-dimethylaminopropyl)carbodiimide hydrochloride (120 mg), 1-hydroxybenzotriazole (5.0 mg), triethylamine (0.8 mL) and N,N-dimethylformamide (5 mL) and in the same manner as in Example C-53(ii), the title compound (43 mg) was obtained as crystals.

¹H-NMR (DMSO-d₆) δ: 0.30-0.57 (4H, m), 1.11-1.35 (1H, m), 3.11 (3H, s), 3.40-3.50 (2H, m), 3.79-3.81 (2H, m), 4.07 (2H, s), 4.54-4.59 (2H, m), 6.32-6.67 (4H, m), 7.19-7.67 (2H, m), 8.02 (2H, s), 8.41 (1H, s), 8.68(1H, br s), 8.80 (1H, s).

### Example C-93

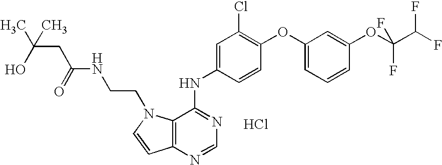

### Production of N-{2-[4-({3-chloro-4-[3-(1,1,2,2-tetrafluoroethoxy)phenoxy]phenyl}amino)-5H-pyrrolo[3,2-d]pyrimidin-5-yl]ethyl}-3-hydroxy-3-methylbutanamide hydrochloride

### (i) Production of 2-chloro-4-nitro-1-[3-(1,1,2,2-tetrafluoroethoxy)phenoxy]benzene

A mixture of 3-chloro-4-fluoronitrobenzene (5 g) and 3-(1,1,2,2-tetrafluoroethoxy)phenol (6 g) was lo dissolved in N,N-dimethylformamide (28 mL), potassium carbonate (5.92 g) was added thereto, and the mixture was stirred at room temperature for 15 hr. Water (200 mL) was added to the reaction mixture, and the mixture was extracted with ethyl acetate (200 mL). The organic layer was dried over anhydrous magnesium sulfate and concentrated under reduced pressure. The residue was separated and purified by silica gel column chromatography (hexane:ethyl acetate=90:10→60:40) to give the title compound (10 g) as a yellow oil.

¹H-NMR (CDCl₃) δ: 5.91 (1H, tt, J=3.0 Hz, 53.0 Hz), 6.90-7.10 (3H, m), 7.10-7.15 (1H, m), 7.44 (1H, t, J=8.0 Hz), 8.10 (1H, dd, J=3.0 Hz, 9.0 Hz), 8.39 (1H, d, J=3.0 Hz).

### (ii) Production of 3-chloro-4-[3-(1,1,2,2-tetrafluoroethoxy)phenoxy]aniline

A mixture of 2-chloro-4-nitro-1-[3-(1,1,2,2-tetrafluoroethoxy)phenoxy]benzene (10 g), reduced iron (10.2 g) and calcium chloride (1.7 g) in ethanol (270 mL)/water (30 mL) was stirred with heating at 90° C. for 16 hr. After cooling to room temperature, the mixture was filtered through celite. The filtrate was concentrated under reduced pressure, and the residue was partitioned between ethyl acetate (300 mL)/saturated brine (200 mL). The organic layer was dried over anhydrous magnesium sulfate and concentrated under reduced pressure. The residue was separated and purified by silica gel column chromatography (hexane:ethyl acetate=90:10→60:40) to give the title compound (7.91 g) as colorless crystals.

¹H-NMR (CDCl₃) δ: 3.70 (2H, br s), 5.87 (1H, tt, J=3.0 Hz, 53.0 Hz), 6.58 (1H, dd, J=3.0,Hz, 8.0 Hz), 6.70-6.85 (3H, m), 6.85-7.00 (2H, m), 7.26 (1H, t, J=8.0 Hz).

### (iii) Production of tert-butyl{2-[4-({3-chloro-4-[3-(1,1,2,2-tetrafluoroethoxy)phenoxy]phenyl}amino)-5H-pyrrolo[3,2-d]pyrimidin-5-yl]ethyl}carbamate

tert-Butyl[2-(4-chloro-5H-pyrrolo[3,2-d]pyrimidin-5-yl)ethyl]carbamate (1.0 g) and 3-chloro-4-[3-(1,1,2,2-tetrafluoroethoxy)phenoxy]aniline (1.70 g) were dissolved in isopropyl alcohol (10 mL), and the mixture was stirred at 80° C. for 20 hr. A saturated aqueous sodium hydrogencarbonate solution (50 mL) was added to the reaction mixture and the mixture was extracted with ethyl acetate (100 mL). The organic layer was dried over anhydrous magnesium sulfate and concentrated under reduced pressure. The residue was separated and purified by silica gel column chromatography (hexane:ethyl acetate=80:20→30:100) to give the title compound (2.04 g) as an oil.

¹H-NMR (CDCl₃) δ: 1.49 (9H, s), 3.40-3.60 (2H, m), 4.40-4.60 (2H, m), 5.11 (1H, t, J=6.0 Hz), 5.89 (1H, tt, J=3.0 Hz, 53.0 Hz), 6.60 (1H, d, J=3.0 Hz), 6.80-7.00 (3H, m), 7.09 (1H, d, J=9.0 Hz), 7.19 (1H, d, J=3.0 Hz), 7.31 (1H, t, J=8.0 Hz), 7.89 (1H, dd, J=3.0 Hz, 9.0 Hz), 8.03 (1H, d, J=3.0 Hz), 8.52 (1H, s), 8.64 (1H, br s).

### (iv) Production of 5-(2-aminoethyl)-N-{3-chloro-4-[3-(1,1,2,2-tetrafluoroethoxy)phenoxy]phenyl}-5H-pyrrolo[3,2-d]pyrimidin-4-amine dihydrochloride

tert-Butyl{2-[4-({3⁻chloro-4-[3-(1,1,2,2-tetrafluoroethoxy)phenoxy]phenyl}amino)-5H-pyrrolo[3,2-d]pyrimidin-5-yl]ethyl}carbamate (1.98 g) was dissolved in tetrahydrofuran (47.2 mL), 2N hydrochloric acid (23.6 mL) was added thereto, and the mixture was stirred at 60° C. for 15 hr. The reaction mixture was concentrated under reduced pressure, the residue was dissolved in ethanol (50 mL), and the mixture was concentrated again under reduced pressure. This operation was repeated twice. The residual solid was filtrated with isopropyl alcohol and dried to give the title compound (1.65 g) as a pale-yellow powder.

¹H-NMR (DMSO-d₆) δ: 3.20-3.40 (2H, m), 5.00-5.10 (2H, m), 6.60-7.00 (4H, m), 7.35 (1H, d, J=9.0 Hz), 7.51 (1H, t, J=9.0 Hz), 7.60-7.70 (1H, m), 7.94 (1H, d, J=2.4 Hz), 8.11 (1H, d, J=3.0 Hz), 8.40 (3H, br s), 8.75 (1H, s).

### (v) Production of N-(2-[4-({3-chloro-4-[3-(1,1,2,2-tetrafluoroethoxy)phenoxy]phenyl)amino)-5H-pyrrolo[3,2-d]pyrimidin-5-yl]ethyl}-3-hydroxy-3-methylbutanamide hydrochloride

5-(2-Aminoethyl)-N-{3-chloro-4-[3-(1,1,2,2-tetrafluoroethoxy)phenoxy]phenyl}-5H-pyrrolo[3,2-d]pyrimidin-4-amine dihydrochloride (204 mg), 3-hydroxy-3-methylbutanoic acid (63.5 mg), N,N-dimethylformamide (6.9 mL), 1-hydroxybenzotriazole (72.5 mg), triethylamine (0.15 mL) and 1-ethyl-3-(3-dimethylaminopropyl)carbodiimide hydrochloride (103 mg) were stirred at room temperature for 16 hr. The mixture was partitioned between ethyl acetate (80 mL)/water (50 mL), the organic layer was dried over anhydrous magnesium sulfate and concentrated under reduced pressure. The residue was separated and purified by silica gel column chromatography (ethyl acetate:methanol=100:0→485:15) and basic silica gel column chromatography (ethyl acetate:methanol=100:0→85:15). The residue was dissolved in ethyl acetate (5 mL), treated with 4N hydrogen chloride/ethyl acetate solution (0.18 mL), and crystallized train diisopropyl ether/ethyl acetate to give the title compound (134 mg) as white crystals.

¹H-NMR (DMSO-d₆) δ: 1.11 (6H, s), 2.20 (2H, s), 3.44-3.56 (2H, m), 4.68 (2H, t, J=6.0 Hz), 6.60-7.10 (5H, m), 7.35 (1H, d, J=9.0 Hz), 7.51 (1H, t, J=8.0 Hz), 7.73 (1H, dd, J=3.0 Hz, 9.0 Hz), 7.98 (2H, dd, J=3.0 Hz, 7.0 Hz), 8.43 (1H, m), 8.74 (1H, s).

### Example C-94

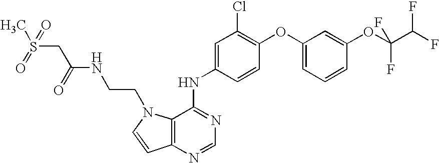

### Production of N-{2-[4-({3-chloro-4-[3-(1,1,2,2-tetrafluoroethoxy)phenoxy]phenyl}amino)-5H-pyrrolo[3,2-d]pyrimidin-5-yl]ethyl}-2-(methylsulfonyl)acetamide

5-(2-Aminoethyl)-N-{3-chloro-4-[3-(1,1,2,2-tetrafluoroethoxy)phenoxy]phenyl}-5H-pyrrolo[3,2-d]pyrimidin-4-amine dihydrochloride (204 mg), methylsulfonylacetic acid (74.2 mg), N,N-dimethylformamide (6.9 mL), 1-hydroxybenzotriazole (72.5 mg), triethylamine (0.15 mL) and 1-ethyl-3-(3-dimethylaminopropyl)carbodiimide hydrochloride (103 mg) were stirred at room temperature for 16 hr. The mixture was partitioned between ethyl acetate (80 mL)/water (50 mL). The organic layer was dried over anhydrous magnesium sulfate and concentrated under reduced pressure. The residue was separated and purified by silica gel column chromatography (ethyl acetate:methanol=100:0→85:15) and basic silica gel column chromatography (ethyl acetate:methanol=100:0→85:15) to give the title compound (140 mg) as pale-yellow crystals.

¹H-NMR (DMSO-d₆) δ: 3.10 (3H, s), 3.40-3.50 (2H, m), 4.05 (2H, s), 4.56 (2H, t, J=7.0 Hz), 6.50 (1H, d, J=3.0 Hz), 6.60-7.10 (4H, m), 7.29 (1H, d, J=9.0 Hz), 7.47 (1H, t, J=8.0 Hz), 7.63 (1H, d, J=3.0 Hz), 7.76 (1H, dd, J=2.0 Hz, 9.0 Hz), 7.98 (1H, d, J=3.0 Hz), 8.35 (1H, s), 8.68 (1H, m), 8.71 (1H, br s).

### Example C-95

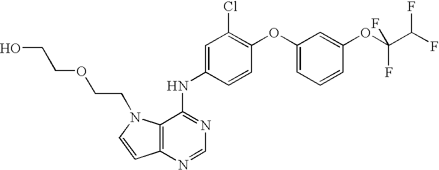

### Production of 2-{2-[4-({3-chloro-4-[3-(1,1,2,2-tetrafluoroethoxy)phenoxy]phenyl}amino)-5H-pyrrolo[3,2-d]pyrimidin-5-yl]ethoxy}ethanol

2-[2-(4-Chloro-5H-pyrrolo[3,2-d]pyrimidin-5-yl)ethoxy]ethyl benzoate (150 mg) and 3-chloro-4-[3-(1,1,2,2-tetrafluoroethoxy)phenoxy]aniline (217 mg) were stirred in isopropyl alcohol (3 mL) at 80° C. for 16 hr. A saturated aqueous sodium hydrogencarbonate solution (40 mL) was added to the reaction mixture and the mixture was extracted with ethyl acetate (80 mL). The organic layer was dried over anhydrous magnesium sulfate and concentrated under reduced pressure. The residue was separated and purified by silica gel column chromatography (hexane:ethyl acetate=90:10→0:100). The obtained oil residue was dissolved in methanol (1.89 mL), 1N aqueous sodium hydroxide solution (0.433 mL) was added and the mixture was stirred at room temperature stirred for 1 hr. 1N hydrochloric acid (0.433 mL) was added to the mixture and the mixture was partitioned between ethyl acetate (50 mL) and saturated brine (20 mL). The organic layer was dried over anhydrous magnesium sulfate and concentrated under reduced pressure. The residue was separated and purified by silica gel column chromatography (ethyl acetate:methanol=100:0→390:10) and basic silica gel column chromatography (ethyl acetate:methanol=100:0→90:10) to give the title compound (136 mg) as white crystals.

¹H-NMR (DMSO-d₆) δ: 3.49 (4H, br s), 3.83 (2H, t, J=4.5 Hz), 4.64 (2H, t, J=4.5 Hz), 4.73 (1H, t, J=4.5 Hz), 6.52 (1H, d, J=3.0 Hz), 6.76 (1H, m), 6.79 (1H, tt, J=3.0 Hz, 52.0 Hz), 6.91 (1H, dd, J=2.0 Hz, 8.0 Hz), 7.02 (1H, d, J=9.0 Hz), 7.27 (1H, d, J=9.0 Hz), 7.47 (1H, t, J=8.0 Hz), 7.60-7.70 (2H, m), 8.01 (1H, d, J=2.0 Hz), 8.34 (1H, s), 8.99 (1H, br s).

### Example C-96

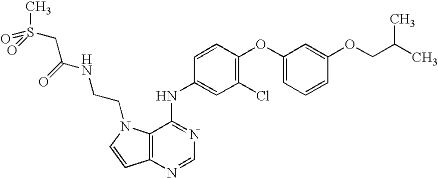

### Production of N-[2-(4-{[3-chloro-4-(3-isobutoxyphenoxy)phenyl]amino}-5H-pyrrolo[3,2-d]pyrimidin-5-yl)ethyl]-2-(methylsulfonyl)acetamide

### (i) Production of 3-chloro-4-(3-isobutoxyphenoxy)aniline

To a solution of 3-(2-chloro-4-nitrophenoxy)phenol (1.00 g) and 1-iodo-2-methylpropane (1.5 mL) in N,N-dimethylformamide (15 mL) was added potassium carbonate (1.50 g) and the mixture was stirred at room temperature for 12 hr. Under ice-cooling, brine was added to the reaction mixture, and the mixture was extracted twice with ethyl acetate, and the organic layer was dried over anhydrous magnesium sulfate. After concentration under reduced pressure; the residue was separated and purified by silica gel column chromatography (eluent, hexane:ethyl acetate=1:0→1:1). The obtained crude product was dissolved in 15% water-containing ethanol (30 mL), reduced iron (750 mg) and calcium chloride (70 mg) were added, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→0:1) to give the title compound (390 mg) as a brown oil.

¹H-NMR (DMSO-d₆) δ: 0.94-0.96 (6H, m), 1.90-2.01 (1H, m), 3.67-3.69 (2H, m), 5.33 (2H, s), 6.32-7.34 (2H, m), 6.53-6.59 (2H, m), 6.71-6.72 (1H, m), 6.89-6.91 (1H, m), 7.14-7.20 (1H, m).

### (ii) Production of N-[2-(4-{[3-chloro-4-(3-isobutoxyphenoxy)phenyl]amino}-5H-pyrrolo[3,2-d]pyrimidin-5-yl)ethyl]-2-(methylsulfonyl)acetamide

Using 3-chloro-4-(3-isobutoxyphenoxy)aniline (1.00 g), tert-butyl[2-(4-chloro-5H-pyrrolo[3,2-d]pyrimidin-5-yl)ethyl]carbamate (1.02 g), isopropyl alcohol (25 mL), methanol (35 mL), 4N hydrogen chloride/ethyl acetate solution (10 mL), methylsulfonylacetic acid (870 mg), 1-ethyl-3-(3-dimethylaminopropyl)carbodiimide hydrochloride (2.50 g), 1-hydroxybenzotriazole (100 mg), triethylamine (4.1 mL) and N,N-dimethylformamide (100 mL) and in the same manner as in Example C-53(ii), the title compound (933 mg) was obtained as crystals.

¹H-NMR (DMSO-d₆) δ: 0.95-0.97 (6H, m), 1.95-2.04 (1H, m), 3.09 (3H, s), 3.40-3.50 (2H, m), 3.72-3.74 (2H, m), 4.04 (2H, s), 4.54-4.59 (2H, m), 6.44-7.27 (6H, m), 7.61-7.75 (2H, m), 7.94 (114, s), 8.33 (1H, s), 8.66 (2H, s).

### Example C-97

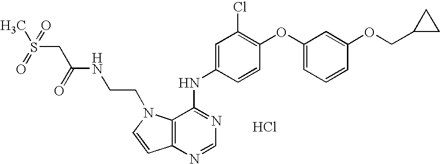

### Production of N-{2-[4-({3-chloro-4-[3-(cyclopropylmethoxy)phenoxy]phenyl}amino)-5H-pyrrolo[3,2-d]pyrimidin-5-yl]ethyl}-2-(methylsulfonyl)acetamide hydrochloride

### (i) Production of 2-chloro-1-[3-(cyclopropylmethoxy)phenoxy]-4-nitrobenzene

To a solution of 3-(2-chloro-4-nitrophenoxy)phenol (2.92 g) in N,N-dimethylformamide (10.9 mL) were added cesium carbonate (5.39 g) and 1-(bromomethyl)cyclopropane (1.6 mL) and the mixture was stirred at room temperature for 16 hr. The mixture was partitioned between ethyl acetate (150 mL)/water (80 mL). The organic layer was dried over anhydrous magnesium sulfate and concentrated under reduced pressure. The residue was separated and purified by silica gel column chromatography (hexane:ethyl acetate=100:0→70:30) to give the title compound (3.17 g) as white crystals.

¹H-NMR (CDCl₃) δ: 0.30-0.40 (2H, m), 0.60-0.70 (2H, m), 1.20-1.40 (1H, m), 3.79 (2H, d, J=7.0 Hz), 6.60-6.70 (2H, m), 6.70-6.82 (1H, m), 6.92(1H, d, J=9.0 Hz), 7.29 (1H, m), 8.00-8.10 (1H, m), 8.36 (1H, d, J=3.0 Hz).

### (ii) Production of 3-chloro-4-[3-(cyclopropylmethoxy)phenoxy]aniline

2-Chloro-1-[3-(cyclopropylmethoxy)phenoxy]-4-nitrobenzene (3.1 g) was dissolved in ethanol (97 mL)/water (11 mL), reduced iron (3.61 g) and calcium chloride (717 mg) were added thereto, and the mixture was stirred at 90° C. for 16 hr. The solid was removed by filtration through celite, and the filtrate was concentrated under reduced pressure. Ethyl acetate (150 mL) was added to the residue, and the mixture was washed with saturated brine (100 mL). The organic layer was dried over magnesium sulfate, concentrated under reduced pressure, and the residue was separated and purified by silica gel column chromatography (eluent, hexane:ethyl acetate=90:10→60:40) to give the title compound (2.50 g) as an oil.

¹H-NMR (CDCl₃) δ: 0.20-0.40 (2H, m), 0.50-0.70 (2H, m), 1.10-1.30 (1H, m), 3.66 (2H, br s), 3.74 (2H, d, J=7.0 Hz), 6.40-6.50 (2H, m), 6.50-6.60 (2H, m), 6.76 (1H, d, J=3.0 Hz), 6.85 (1H, d, J=9.0 Hz), 7.10-7.20 (1H, m).

### (iii) Production of tert-butyl{2-[4-({3-chloro-4-[3-(cyclopropylmethoxy)phenoxy]phenyl}amino)-5H-pyrrolo[3,2-d]pyrimidin-5-yl]ethyl}carbamate

tert-Butyl[2-(4-chloro-5H-pyrrolo[3,2-d]pyrimidin-5-yl)ethyl]carbamate (0.5 g) and 3-chloro-4-[3-(cyclopropylmethoxy)phenoxy]aniline (732 mg) were dissolved in isopropyl alcohol (5 mL), and the mixture was stirred at 80° C. for 20 hr. A saturated aqueous sodium hydrogencarbonate solution (60 mL) was added to the reaction mixture, and the mixture was extracted with ethyl acetate (80 mL). The organic layer was dried over anhydrous magnesium sulfate and concentrated under reduced pressure. The residue was separated and purified by silica gel column chromatography (hexane:ethyl acetate-90:10→40:100) to give the title compound (742 mg) as a white powder.

¹H-NMR (CDCl₃) δ: 0.30-0.40 (2H, m), 0.60-0.70 (2H, m), 1.10-1.30 (1H, m), 1.49 (9H, s), 3.40-3.60 (2H, m), 3.76 (2H, d, J=7.0 Hz), 4.40-4.50 (2H, m), 5.21 (1H, t, J=5.0 Hz), 6.50-6.70 (4H, m), 7.04 (1H, d, J=9.0 Hz), 7.10-7.30 (2H, m), 7.82 (1H, dd, J=2.0 Hz, 9.0 Hz), 7.98 (1H, d, J=3.0 Hz), 8.49 (1H, s), 8.59 (1H, br s).

### (iv) Production of 5-(2-aminoethyl)-N-{3-chloro-4-[3-(cyclopropylmethoxy)phenoxy]phenyl}-5H-pyrrolo[3,2-d]pyrimidin-4-amine dihydrochloride

tert-Butyl{2-[4-({3-chloro-4-[3-(cyclopropylmethoxy)phenoxy]phenyl}amino)-5H-pyrrolo[3,2-d]pyrimidin-5-yl]ethyl}carbamate (700 mg) was dissolved in tetrahydrofuran (18 mL), 2N hydrochloric acid (9.0 mL) was added thereto, and the mixture was stirred at 60° C. for 15 hr. The reaction mixture was concentrated under reduced pressure, the residue was dissolved in ethanol (50 mL), and the solution was concentrated again under reduced pressure. This operation was repeated twice. The precipitated solid was collected with isopropyl alcohol and dried to give the title compound (599 mg) as a pale-yellow powder.

¹H-NMR (DMSO-d₆) δ: 0.20-0.40 (2H, m), 0.50-0.60 (2H, m), 1.10-1.40 (1H, m), 3.20-3.40 (2H, m), 3.80 (2H, d, J=7.0 Hz), 5.00-5.10 (2H, m), 6.40-6.60 (2H, m), 6.60-6.80 (2H, m), 7.10-7.30 (2H, m), 7.50-7.70 (1H, m), 7.88 (1H, m), 8.08 (1H, m), 8.37 (3H, br s), 8.73 (1H, s), 10.19 (1H, br s).

### (v) Production of N-{2-[4-({3-chloro-4-[3-(cyclopropylmethoxy)phenoxy]phenyl}amino)-5H-pyrrolo[3,2-d]pyrimidin-5-yl]ethyl}-2-(methylsulfonyl)acetamide hydrochloride

5-(2-Aminoethyl)-N-{3-chloro-4-[3-(cyclopropylmethoxy)phenoxy]phenyl}-5H-pyrrolo[3,2-d]pyrimidin-4-amine dihydrochloride (187 mg), methylsulfonylacetic acid (74.2 mg), N,N-dimethylformamide (6.9 mL), 1-hydroxybenzotriazole (72.5 mg), triethylamine (0.15 mL) and 1-ethyl-3-(3-dimethylaminopropyl)carbodiimide hydrochloride (103 mg) were stirred at room temperature for 16 hr. The mixture was partitioned between ethyl acetate (80 mL)/water (50 mL). The organic layer was dried over anhydrous magnesium sulfate and concentrated/under reduced pressure. The residue was separated and purified by silica gel column chromatography (ethyl acetate:methanol=100:0→85:15) and basic silica gel column chromatography (ethyl acetate:methanol=100:0→485:15). The obtained compound was dissolved in ethyl acetate (4 mL), treated with 4N hydrogen chloride/ethyl acetate solution (0.18 mL), and crystallized from diisopropyl ether/ethyl acetate to give the title compound (167 mg) as white crystals.

¹H-NMR (DMSO-d₆) δ: 0.20-0.40 (2H, m), 0.50-0.60 (2H, m), 1.10-1.40 (1H, m), 3.05 (3H, s), 3.50-3.60 (2H, m), 3.80 (2H, d, J=7.0 Hz), 4.07 (2H, s), 4.73 (1H, t, J=7.0 Hz), 6.40-6.55 (2H, m), 6.60-6.75 (2H, m), 7.20-7.30 (2H, m), 7.63 (1H, dd, J=2.0 Hz, 9.0 Hz), 7.90 (1H, d, J=2.0 Hz), 7.96 (1H, d, J=3.0 Hz), 8.72 (1H, s), 8.87 (1H, m), 10.05 (1H, br s).

### Example C-98

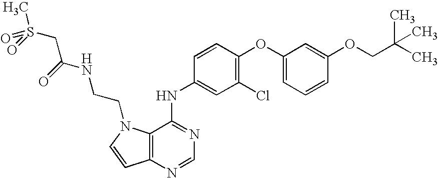

### Production of N-{2-[4-({3-chloro-4-[3-(2,2-dimethylpropoxy)phenoxy]phenyl}amino)-5H-pyrrolo[3,2-d]pyrimidin-5-yl]ethyl}-2-(methylsulfonyl)acetamide

### (i) Production of 2-chloro-1-[3-(2,2-dimethylpropoxy)phenoxy]-4-nitrobenzene

To a solution of 3-(2-chloro-4-nitrophenoxy)phenol (1.0 g) in N,N-dimethylformamide (10 mL) were added potassium hydroxide (423 mg) and tris(dimethylamino)(2,2-dimethylpropoxy)phosphonium hexafluorophosphate (2.99 g) and the mixture was stirred at 180° C. for 30 min. Under ice-cooling, water was added to the reaction mixture, and the mixture was extracted twice with ethyl acetate, and the organic layer was dried over anhydrous magnesium sulfate. After concentration under reduced pressure, the residue was separated and purified by silica gel column chromatography (eluent, hexane:ethyl acetate=1:0→1:1) to give the title compound (180 mg) as crystals.

¹H-NMR (CDCl₃) δ: 1.03 (9H, s), 3.58(2H, s), 6.63-6.65 (2H, m), 6.80-6.94 (2H, m), 7.30-7.34 (1H, m), 8.04-8.08 (1H, m), 8.37-8.38 (1H, m).

### (ii) Production of 3-chloro-4-[3-(2,2-dimethylpropoxy)phenoxy]aniline

2-Chloro-1-[3-(2,2-dimethylpropoxy)phenoxy]-4-nitrobenzene (210 mg) was dissolved in 15% water-containing ethanol (7 mL), reduced iron (150 mg) and calcium chloride (20 mg) were added, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→4.0:1) to give the title compound (75 mg) as a brown oil.

¹H-NMR (DMSO-d₆) δ: 0.98 (9H, s), 3.92 (2H, s), 5.34 (2H, s), 6.31-7.33 (2H, m), 6.52-6.58 (2H, m), 6.70-6.72 (1H, m), 6.88-6.92 (1H, m), 7.13-7.20 (1H, m).

### (iii) Production of N-{2-[4-({3-chloro-4-[3-(2,2-dimethylpropoxy)phenoxy]phenyl}amino)-5H-pyrrolo[3,2-d]pyrimidin-5-yl]ethyl}-2-(methylsulfonyl)acetamide

Using 3-chloro-4-[3-(2,2-dimethylpropoxy)phenoxy]aniline (75 mg), tert-butyl[2-(4-chloro-5H-pyrrolo[3,2-d]pyrimidin-5-yl)ethyl]carbamate (73 mg), isopropyl alcohol (10 mL), methanol (10 mL), 4N hydrogen chloride/ethyl acetate solution (5 mL), methylsulfonylacetic acid (50 mg), 1-ethyl-3-(3-dimethylaminopropyl)carbodiimide hydrochloride (120 mg), 1-hydroxybenzotriazole (10 mg), triethylamine (0.7 mL) and N,N-dimethylformamide (10 mL) and in the same manner as in Example C-53(ii), the title compound (62 mg) was obtained as crystals.

¹H-NMR (DMSO-d₆) δ: 0.98 (9H, s), 3.09 (3H, s), 3.45-3.49 (2H, m), 3.62 (2H, s), 4.05 (2H, s), 4.54-4.59 (2H, m), 6.44-7.28 (6H, m), 7.66-7.94 (3H, m), 8.39 (1H, s), 8.67 (1H, br s), 8.81 (1H, s).

### Example C-99

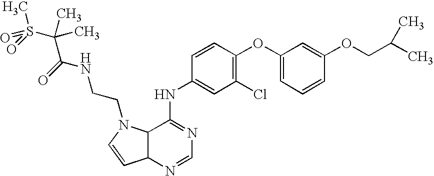

### Production of N-[2-(4-{[3-chloro-4-(3-isobutoxyphenoxy)phenyl]amino}-5H-pyrrolo[3,2-d]pyrimidin-5-yl)ethyl]-2-methyl-2-(methylsulfonyl)propanamide

Using 3-chloro-4-(3-isobutoxyphenoxy)aniline (180 mg), tert-butyl[2-(4-chloro-5H-pyrrolo[3,2-d]pyrimidin-5-yl)ethyl]carbamate (185 mg), isopropyl alcohol (5.0 mL), methanol (6.0 mL), 4N hydrogen chloride/ethyl lo acetate solution (3.0 mL), 2-methyl-2-(methylsulfonyl)propanoic acid (160 mg), 1-ethyl-3-(3-dimethylaminopropyl)carbodiimide hydrochloride (320 mg), 1-hydroxybenzotriazole (20 mg), triethylamine (1.0 mL) and N,N-dimethylformamide (25 mL) and in the same manner as in Example C-53(ii), the title compound (146 mg) was obtained as crystals.

¹H-NMR (DMSO-d₆) δ: 0.95-0.97 (6H, m), 1.41 (6H, s), 1.95-2.04 (1H, m), 2.95 (3H, s), 3.40-3.50 (2H, m), 3.72-3.74 (2H, m), 4.54-4.59 (2H, m), 6.44-7.27 (6H, m), 7.56-7.97 (3H, m), 8.19-8.22 (1H, m), 8.33 (1H, s), 8.65(1H, s).

### Example C-100

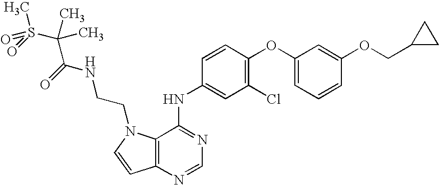

### Production of N-{2-[4-({3-chloro-4-[3-(cyclopropylmethoxy)phenoxy]phenyl}amino)-5H-pyrrolo[3,2-d]pyrimidin-5-yl]ethyl}-2-methyl-2-(methylsulfonyl)propanamide

To a solution of 5-(2-aminoethyl)-N-{3-chloro-4-[3-(cyclopropylmethoxy)phenoxy]phenyl}-5H-pyrrolo[3,2-d]pyrimidin-4-amine dihydrochloride (200 mg), 2-methyl-2-(methylsulfonyl)propanoic acid (170′mg) and 1-hydroxybenzotriazole (20 mg) in N,N-dimethylformamide (25 mL) were added triethylamine (0.87 mL) and 1-ethyl-3-(3-dimethylaminopropyl)carbodiimide hydrochloride (320 mg) under ice-cooling and the mixture was stirred at room temperature for 15 hr. Water was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→90:10) and further recrystallized from ethyl acetate/diisopropyl ether to give the title compound (160 mg) as colorless crystals.

¹H-NMR (DMSO-d₆) δ: 0.29-0.58 (4H, m), 1.11-1.28 (1H, m), 1.41 (6H, s), 2.95 (3H, s), 3.44-3.50 (2H, m), 3.78-3.80 (2H, m), 4.55-4.59 (2H, m), 6.32-6.68 (4H, m), 7.16-7.96 (5H, m), 8.20 (1H, br s), 8.33 (1H, s), 8.65(1H, br s).

### Example C-101

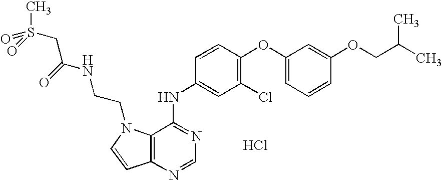

### Production of N-[2-(4-{[3-chloro-4-(3-isobutoxyphenoxy)'phenyl]amino}-5H-pyrrolo[3,2-d]pyrimidin-5-yl)ethyl]-2-(methylsulfonyl)acetamide hydrochloride

N-[2-(4-{[3-Chloro-4-(3-isobutoxyphenoxy)phenyl]amino}-5H-pyrrolo[3,2-d]pyrimidin-5-yl)ethyl]-2-(methylsulfonyl)acetamide (1.00 g) was dissolved in ethyl acetate (10 mL), 4N hydrogen chloride/ethyl acetate solution (5.0 mL) was added thereto, and the precipitated solid was collected by filtration. The solid was washed with diisopropyl ether to give the title compound (1.03 g) as colorless crystals.

¹H-NMR (DMSO-d₆) δ: 0.95-0.98 (6H, m), 1.92-2.04 (1H, m), 3.05 (3H, s), 3.40-3.56 (2H, m), 3.72-3.75 (2H, m), 4.07 (2H, s), 4.71-4.75 (2H, m), 6.47-7.32 (6H, m), 7.62-7.97 (3H, m), 8.73 (1H, s), 8.83-8.88 (1H, m), 10.03 (1H, s).

### Example C-102

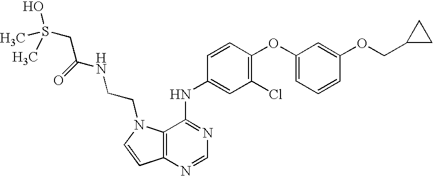

### Production of N-{2-[4-({3-chloro-4-[3-(cyclopropylmethoxy)phenoxy]phenyl}amino)-5H-pyrrolo[3,2-d]pyrimidin-5-yl]ethyl}-3-hydroxy-3-methylbutanamide

To a solution of 5-(2-aminoethyl)-N-{3-chloro-4-[3-(cyclopropylmethoxy)phenoxy]phenyl}-5H-pyrrolo[3,2-d]pyrimidin-4-amine dihydrochloride (100 mg), 3-hydroxy-3-methylbutanoic acid (293 mg) and 1-hydroxybenzotriazole (10 mg) in tetrahydrofuran (15 mL) were added triethylamine (0.3 mL) and 1-ethyl-3-(3-dimethylaminopropyl)carbodiimide hydrochloride (216 mg) under ice-cooling, and the mixture was stirred at room temperature for 3 hr. Water was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:methanol=100:0→90:10) and further recrystallized from ethyl acetate/diisopropyl ether to give the title compound (66 mg) as colorless crystals.

¹H-NMR (DMSO-d₆) δ: 0.29-0.58 (4H, m), 1.11-1.28 (1H, m), 1.16 (6H, s), 2.20 (2H, s), 3.35-3.41 (2H, m), 3.78-3.80 (2H, m), 4.49-4.54 (2H, m), 4.66 (1H, s), 6.43-7.26 (6H, m), 7.63-8.00 (3H, m), 8.25 (1H, br s), 8.32 (1H, s), 8.86(1H, br s).

### Example C-103

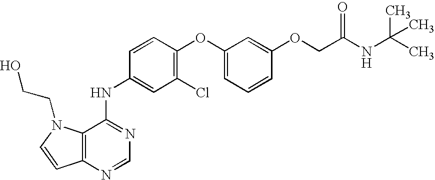

### Production of N-(tert-butyl)-2-[3-(2-chloro-4-{[5-(2-hydroxyethyl)-5H-pyrrolo[3,2-d]pyrimidin-4-yl]amino}phenoxy)phenoxy]acetamide

### (i) Production of 2-[3-(4-amino-2-chlorophenoxy)phenoxy]-N-(tert-butyl)acetamide

To a solution of 3-(2-chloro-4-nitrophenoxy)phenol (1.0 g) in N,N-dimethylformamide (10 mL) were added ethyl bromoacetate (1.26 g) and potassium carbonate (1.20 g) and the mixture was stirred at room temperature for 18 hr. Under ice-cooling, brine was added to the reaction mixture; and the mixture was extracted twice with ethyl acetate and dried over anhydrous magnesium sulfate. After concentration under reduced pressure, the residue was dissolved in tetrahydrofuran (20 mL), 1N aqueous sodium hydroxide solution (5.0 mL) was added thereto and the mixture was stirred at room temperature for 12 hr. The reaction mixture was neutralized with 1N hydrochloric acid, and aqueous sodium bicarbonate and brine were added thereto. The mixture was extracted with ethyl acetate, and the organic layer was dried over anhydrous magnesium sulfate and concentrated under reduced pressure. To a solution of the obtained residue, 2-methylpropan-2-amine (1.0 mL) and 1-hydroxybenzotriazole (10 mg) in tetrahydrofuran (15 mL) were added triethylamine (1.3 mL) and 1-ethyl-3-(3-dimethylaminopropyl)carbodiimide hydrochloride (1.50 g) under ice-cooling, and the mixture was stirred at room temperature for 3 hr. Water was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, hexane:ethyl acetate=1:0→1:1). The obtained crude product was dissolved in 15% water-containing ethanol (23 mL), reduced iron (550 mg) and calcium chloride (70 mg) were added thereto, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→0:1) to give the title compound (375 mg) as a brown oil.

¹H-NMR (DMSO-d₆) δ: 1.29 (9H, s), 4.35 (2H, s), 5.35 (2H, s), 6.31-7.34 (2H, m), 6.52-6.58 (2H, m), 6.70-6.72 (1H, m), 6.88-6.93 (1H, m), 7.13-7.20 (1H, m).

### (ii) Production of N-(tert-butyl)-2-[3-(2-chloro-4-{[5-(2-hydroxyethyl)-5H-pyrrolo[3,2-d]pyrimidin-4-yl]amino}phenoxy)phenoxy]acetamide

Using 2-[3-(4-amino-2-chlorophenoxy)phenoxy]-N-(tert-butyl)acetamide (100 mg), 2-(4-chloro-5H-pyrrolo[3,2-d]pyrimidin-5-yl)ethyl benzoate (87 mg), isopropyl alcohol (5.0 mL), 1N aqueous sodium hydroxide solution (2.0 mL) and methanol (5.0 mL) and in the same manner as in Example C-57, the title compound (73 mg) was obtained as crystals.

¹H-NMR (DMSO-d₆) δ: 1.27 (9H, s), 3.86-3.89 (2H, m), 4.37 (2H, s), 4.51-4.55 (2H, m), 6.48-7.66 (9H, m), 7.95-8.97 (1H, m), 8.33 (1H, s), 9.87(1H, br s).

### Example C-104

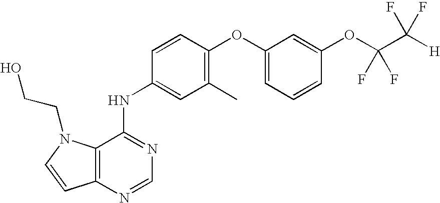

### Production of 2-[4-({3-methyl-4-[3-(1,1,2,2-tetrafluoroethoxy)phenoxy]phenyl}amino)-5H-pyrrolo[3,2-d]pyrimidin-5-yl]ethanol

### (i) Production of 2-methyl-4-nitro-1-[3-(1,1,2,2-tetrafluoroethoxy)phenoxy]benzene

A mixture of 2-fluoro-5-nitrotoluene (2.10 g) and 3-(1,1,2,2-tetrafluoroethoxy)phenol (3.01 g) was dissolved in N,N-dimethylformamide (13 mL), potassium carbonate (3.04 g) was added thereto, and the mixture was stirred at room temperature for 15 hr. Water was added to the reaction mixture, and the mixture was extracted with ethyl acetate. The organic layer was washed successively with water and'saturated brine, dried over anhydrous magnesium sulfate, and concentrated under reduced pressure. The residue was separated and purified by silica gel column chromatography (hexane:ethyl acetate=100:0→80:20) to give the title compound (4.05 g) as a yellow oil.

¹H-NMR (CDCl₃) δ: 2.39.(3H, s), 5.91 (1H, tt, J=2.8 Hz, 53.0 Hz), 6.87 (1H, d, J=9.0 Hz), 6.89-6.98 (2H, m), 7.02-7.14 (1H, m), 7.41 (1H, t, J=8.2 Hz), 8.04 (1H, dd, J=2.6 Hz, 9.0 Hz), 8.18 (1H, d, J=2.6 Hz).

### (ii) Production of 3-methyl-4-[3-(1,1,2,2-tetrafluoroethoxy)phenoxy]aniline

Using 2-methyl-4-nitro-1-[3-(1,1,2,2-tetrafluoroethoxy)phenoxy]benzene (4.04 g), reduced iron (3.45 g), calcium chloride (691 mg) and ethanol (108 mL)/water (12 mL) and in the same manner as in Example C-72(ii), the title compound (3.45 g) was obtained as a brown oil.

¹H-NMR (CDCl₃) δ: 2.09 (3H, s), 3.59 (2H, br s), 5.86 (1H, tt, J=2.9 Hz, 53.0 Hz), 6.53 (1H, dd, J=3.0 Hz, 8.7 Hz), 6.59 (1H, d, J=3.0 Hz), 6.67 (1H, t, J=2.2 Hz), 6.72-6.77 (1H, m), 6.80 (1H, d, J=8.7 Hz), 6.81-6.86 (1H, m), 7.19-7.27 (1H, m).

### (iii) Production of 2-[4-({3-methyl-4-[3-(1,1,2,2-tetrafluoroethoxy)phenoxy]phenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (102 mg), 3-methyl-4-[3-(1,1,2,2-tetrafluoroethoxy)phenoxy]aniline (110 mg), isopropyl alcohol (3 mL) and 1N aqueous sodium hydroxide solution (1.5 mL) and in the same manner as in Example C-72(iii), the title compound (114 mg) was obtained as white crystals.

¹H-NMR (CDCl₃) δ: 2.23 (3H, s), 4.14 (2H, t, J=4.5 Hz), 4.32-4.42 (2H, m), 5.89 (1H, tt, J=2.9 Hz, 53.0 Hz), 6.15 (1H, d, J=3.4 Hz), 6.75-7.02 (5H, m), 7.20-7.34 (1H, m), 7.40-7.52 (2H, m), 8.24 (1H, s), 9.30 (1H, s).

### Example C-105

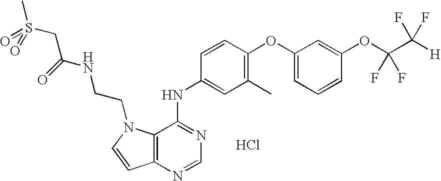

### Production of 2-(methylsulfonyl)-N-{2-[4-({3-methyl-4-[3-(1,1,2,2-tetrafluoroethoxy)phenoxy]phenyl}amino)-5H-pyrrolo[3,2-d]pyrimidin-5-yl]ethyl}acetamide hydrochloride

### (i) Production of tert-butyl{2-[4-({3-methyl-4-[3-(1,1,2,2-tetrafluoroethoxy)phenoxy]phenyl}amino)-5H-pyrrolo[3,2-d]pyrimidin-5-yl]ethyl}carbamate

tert-Butyl[2-(4-chloro-5H-pyrrolo[3,2-d]pyrimidin-5-yl)ethyl]carbamate (304 mg) and 3-methyl-4-[3-(1,1,2,2-tetrafluoroethoxy)phenoxy]aniline (331 mg) were dissolved in isopropyl alcohol (10 mL), and the mixture was stirred at 70° C. for 20 hr. A saturated aqueous sodium hydrogencarbonate solution was added to the reaction mixture, and the mixture was extracted with ethyl acetate. The organic layer was washed successively with water and saturated brine, dried over anhydrous magnesium sulfate and concentrated under,reduced pressure. The residue was separated and purified by silica gel column chromatography (hexane:ethyl acetate=67:33→10:90) to give the title compound (540 mg)) as a colorless oil.

¹H-NMR (CDCl₃) δ: 1.46 (9H, s), 2.21 (3H, s), 3.43-3.57 (2H, m), 4.40-4.54 (2H, m), 5.00 (1H, t, J=5.5 Hz), 5.88 (1H, tt, J=2.9 Hz, 53.0 Hz), 6.59 (1H, d, J=3.0 Hz), 6.75-6.80 (1H, m), 6.82-6.93, (2H, m), 6.94-7.02 (1H, m), 7.16 (1H, d, J=3.0 Hz), 7.27 (1H, t, J=8.1 Hz), 7.64-7.74 (2H, m), 8.36 (1H, br s), 8.50 (1H, s).

### (ii) Production of 5-(2-aminoethyl)-N-{3-methyl-4-[3-(1,1,2,2-tetrafluoroethoxy)phenoxy]phenyl}-5H-pyrrolo[3,2-d]pyrimidin-4-amine dihydrochloride

tert-Butyl{2-[4-({3-methyl-4-[3-(1,1,2,2-tetrafluoroethoxy)phenoxy]phenyl}amino)-5H-pyrrolo[3,2-d]pyrimidin-5-yl]ethyl}carbamate (537 mg) was dissolved in tetrahydrofuran (2 mL), 6N hydrochloric acid (0.8 mL) was added thereto, and the mixture was stirred at 50° C. for 15 hr. The reaction mixture was concentrated under reduced pressure, the residue was dissolved in ethanol, and the mixture was concentrated again under reduced pressure. The precipitate was collected by filtration to give the title compound (460 mg) as a yellow powder.

¹H-NMR (DMSO-d₆) δ: 2.19 (3H, s), 3.22-3.48 (2H, m), 4.99 (2H, t, J=6.1 Hz), 6.72 (1H, d, J=3.2 Hz), 6.76 (1H, t, J=2.3 Hz), 6.80 (1H, tt, J=3.1 Hz, 52 Hz), 6.90-6.96 (1H, m), 7.00-7.07 (1H, m), 7.11 (1H, d, J=8.7 Hz), 7.47 (1H, dd, J=2.5 Hz, 8.7 Hz), 7.49 (1H, t, J=8.3 Hz), 7.54 (1H, d, J=2.5 Hz), 8.03 (1H, d, J=3.2 Hz), 8.29 (3H, br s), 8.68 (1H, s), 9.87 (1H, br s).

### (iii) Production of 2-(methylsulfonyl)-N-{2-[4-({3-methyl-4-[3-(1,1,2,2-tetrafluoroethoxy)phenoxy]phenyl}amino)-5H-pyrrolo[3,2-d]pyrimidin-5-yl]ethyl}acetamide hydrochloride

Using 5-(2-aminoethyl)-N-{3-methyl-4-[3-(1,1,2,2-tetrafluoroethoxy)phenoxy]phenyl}-5H-pyrrolo[3,2-d]pyrimidin-4-amine dihydrochloride (121 mg), methylsulfonylacetic acid (48.8 mg), tetrahydrofuran (0.6 mL)/N,N-dimethylformamide (0.6 mL), 1-hydroxybenzotriazole (47 mg), triethylamine (0.3 mL) and 1-ethyl-3-(3-dimethylaminopropyl)carbodiimide hydrochloride (64.3 mg) and in the same manner as in Example C-72(i), 2-(methylsulfonyl)-N-{2-[4-({3-methyl-4-[3-(1,1,2,2-tetrafluoroethoxy)phenoxy]phenyl}amino)-5H-pyrrolo[3,2-d]pyrimidin-5-yl]ethyl}acetamide was obtained. The compound was dissolved in ethyl acetate, treated with 4N hydrogen chloride/ethyl acetate solution, and crystallized from ethyl acetate/ethanol to give the title compound (104 mg) as pale-yellow crystals.

¹H-NMR (DMSO-d₆) δ: 2.19 (3H, s), 3.05 (3H, s), 3.49-3.60 (2H, m), 4.07 (2H, m), 4.70 (2H, t, J=6.4 Hz), 6.61-6.99 (1H, m), 6.64 (1H, d, J=3.2 Hz), 6.76 (1H, t, J=2.3 Hz), 6.91-6.96 (1H, m), 6.99-7.07 (1H, m), 7.12 (1H, d, J=8.5 Hz), 7.44-7.61 (3H, m), 7.91 (1H, d, J=3.2 Hz), 8.68 (1H, s), 8.78 (1H, t, J=5.7 Hz), 9.85 (1H, s).

### Example C-106

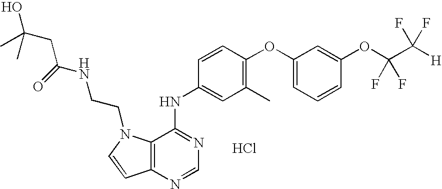

### Production of 3-hydroxy-3-methyl-N-{2-[4-({3-methyl-4-[3-(1,1,2,2-tetrafluoroethoxy)phenoxy]phenyl}amino)-5H-pyrrolo[3,2-d]pyrimidin-5-yl]ethyl}butanamide hydrochloride

Using 5-(2-aminoethyl)-N-{3-methyl-4-[3-(1,1,2,2-tetrafluoroethoxy)phenoxy]phenyl}-5H-pyrrolo[3,2-d]pyrimidin-4-amine dihydrochloride (120 mg), 3-hydroxy-3-methylbutanoic acid (40.5 mg), tetrahydrofuran (0.6 mL)/N,N-dimethylformamide (0.6 mL), 1-hydroxybenzotriazole (47.6 mg), triethylamine (0.3 mL) and 1-ethyl-3-(3′-dimethylaminopropyl)carbodiimide hydrochloride (66.1 mg) and in the same manner as in Example C-72(i), 3-hydroxy-3′-methyl-N-{2-[4-({3-methyl-4-[3-(1,1,2,2-tetrafluoroethoxy)phenoxy]phenyl}amino)-5H-pyrrolo[3,2-d]pyrimidin-5-yl]ethyl}butanamide was obtained. The compound was dissolved in ethyl acetate, treated with 4N hydrogen chloride/ethyl acetate solution, and crystallized from diisopropyl ether/ethyl acetate to give the title compound (108 mg) as white crystals.

¹H-NMR (DMSO-d₆) δ: 1.11 (6H, s), 2.18 (3H, s), 2.20 (2H, s), 3.44-3.56 (2H, m), 4.65 (2H, t, J=6.8 Hz), 6.60-6.99 (1H, m), 6.65 (1H, d, J=3.0 Hz), 6.75 (1H, t, J=2.3 Hz), 6.91-6.96 (1H, m), 6.99-7.06 (1H, m), 7.11 (1H, d, J=8.7 Hz), 7.49 (1H, t, J=8.3 Hz), 7.54 (1H, dd, J=2.5 Hz, 8.7 Hz), 7.60 (1H, d, J=2.5 Hz), 7.95 (1H, d, J=3.0 Hz), 8.37 (1H, t, J=5.5 Hz), 8.68 (1H, s), 10.16 (1H, s).

### Example C-107

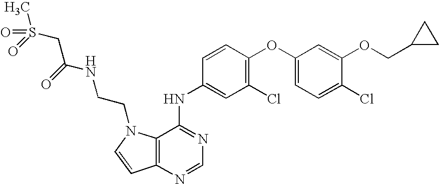

### Production of N-{2-[4-({3-chloro-4-[4-chloro-(cyclopropylmethoxy)phenoxy]phenyl}amino)-5H-pyrrolo[3,2-d]pyrimidin-5-yl]ethyl}-2-(methylsulfonyl)acetamide

### (i) Production of 3-chloro-4-[4-chloro-3-(cyclopropylmethoxy)phenoxy]aniline

To a solution of 3-chloro-4-fluoronitrobenzene (5.00 g), 4-chlorobenzene-1,3-diol (4.11 g) in N,N-dimethylformamide (35 mL) was added potassium carbonate (5.50 g) and the mixture was stirred at room temperature for 5 hr. Under ice-cooling, brine was added to the reaction mixture, and the mixture was extracted twice with ethyl acetate, and the organic layer was dried over anhydrous magnesium sulfate. After concentration under reduced pressure, the residue was separated and purified by silica gel column chromatography (eluent, hexane:ethyl acetate=1:0→1:1). The obtained crude product was dissolved in N,N-dimethylformamide (15 mL), 1-(bromomethyl)cyclopropane (1.3 mL) and potassium carbonate (1.50 g) were added thereto and the mixture was stirred at room temperature for 12 hr. Under ice-cooling, brine was added to the reaction mixture, and the mixture was extracted twice with ethyl acetate, and the organic layer was dried over anhydrous magnesium sulfate. After concentration under reduced pressure, the residue was separated and purified by silica gel column chromatography (eluent, hexane:ethyl acetate=1:0→1:1). The obtained crude product was dissolved in 15% water-containing ethanol (30 mL), reduced iron (550 mg) and calcium chloride (70 mg) were added thereto, and the mixture was stirred at 80° C. for 8 hr. The solid was removed by filtration, and the filtrate was concentrated under reduced pressure. Water was added to the residue, and the mixture was extracted with ethyl acetate. The organic layer was washed with saturated brine and dried over magnesium sulfate. After concentration under reduced pressure, the residue was separated and purified by silica gel column chromatography (eluent, hexane:ethyl acetate=4:1→0:1) to give the title compound (868 mg) as a brown oil.

¹H-NMR (CDCl₃) δ: 0.35-0.68 (4H, m), 0.84-0.90 (1H, m), 3.87 (2H, d, J=6.7 Hz), 5.36 (2H, s), 6.33-7.34 (1H, m), 6.52-6.58 (2H, m), 6.69-6.71 (1H, m), 6.86-6.92 (1H, m), 7.13-7.21 (1H, m).

### (ii) Production of N-{2-[4-({3-chloro-4-[4-chloro-3-(cyclopropylmethoxy)phenoxy]phenyl}amino)-5H-pyrrolo[3,2-d]pyrimidin-5-yl]ethyl}-2-(methylsulfonyl)acetamide

Using 3-chloro-4-[4-chloro-3-(cyclopropylmethoxy)phenoxy]aniline (200 mg), tert-butyl [2-(4-chloro-5H-pyrrolo[3,2-d]pyrimidin-5-yl)ethyl]carbamate (183 mg), isopropyl alcohol (9 mL), methanol (15 mL), 4N hydrogen chloride/ethyl acetate solution (8.0 mL), methylsulfonylacetic acid(170 mg), 1-ethyl-3-(3-dimethylaminopropyl)carbodiimide hydrochloride (300 mg), 1-hydroxybenzotriazole (50 mg), triethylamine (1.1 mL) and N,N-dimethylformamide (10 mL) and in the same manner as in. Example C-53(ii), the title compound (61 mg) was obtained as crystals.

¹H-NMR (CDCl₃) δ: 0.36-0.42 (2H, m), 0.62-0.68 (2H, m), 0.86-0.90 (1H, m), 3.13 (3H, s), 3.68-3.72 (2H, m), 3.86 (2H, d, J=6.7 Hz), 3.98 (2H, s), 4.45-4.52 (2H, m), 6.43-7.28 (6H, m), 7.50 (1H, br s), 7,68-7.72 (1H, m), 7.92 (1H, s), 8.18 (1H, br s), 8.49 (1H, s).

### Example C-108

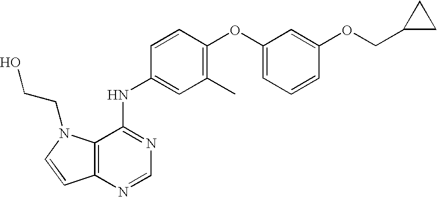

### Production of 2-[4-({4-[3-(cyclopropylmethoxy)phenoxy]-3-methylphenyl}amino)-5H-pyrrolo[3,2-d]pyrimidin-5-yl]ethanol

### (i) Production of 3-(2-methyl-4-nitrophenoxy)phenol

Using 2-fluoro-5-nitrotoluene (10.0 g), resorcinol (24.8 g), potassium carbonate (31.3 g) and N,N-dimethylformamide (120 mL) and in the same manner as in Example C-104(i), the title compound (7.19 g) was obtained as a yellow oil.

¹H-NMR (CDCl₃) δ: 2.39 (3H, s), 5.12 (1H, s), 6.52 (1H, t, J=2.3 Hz), 6.55-6.61 (1H, m), 6.64-6.70 (1H, m), 6.84 (1H, d, J=9.0 Hz), 7.24 (1H, t, J=8.1′Hz), 7.99 (1H, dd, J=2.8 Hz, 9.0 Hz), 8.14 (1H, d, J=2.8 Hz).

### (ii) Production of 1-[3-(cyclopropylmethoxy)phenoxy]-2-methyl-4-nitrobenzene

Using 3-(2-methyl-4-nitrophenoxy)phenol (939 mg), 1-(bromomethyl)cyclopropane (0.55 mL), potassium carbonate (963 mg) and N,N-dimethylformamide (10 mL) and in the same, manner as in Example C-104(i), the title compound (1.02 g) was obtained as a pale-yellow oil.

¹H-NMR (CDCl₃) δ: 0.28-0.42 (2H, m), 0.58-0.71 (2H, m), 1.17-1.36 (1H, m), 2.39 (3H, s), 3.78 (2H, d, J=7.2 Hz), 6.53-6.63 (2H, m), 6.71-6.78 (1H, m), 6.82 (1H, d, J=8.8 Hz), 7.27 (1H, t, J=8.1 Hz), 7.98 (1H, dd, J=2.8 Hz, 8.8 Hz), 8.14 (1H, d, 2.8 Hz).

### (iii) Production of 4-[3-(cyclopropylmethoxy)phenoxy]-3-methylaniline

Using 1-[3-(cyclopropylmethoxy)phenoxy]-2-methyl-4-nitrobenzene (1.01 g), reduced iron (964 mg), calcium chloride (196 mg) and ethanol (36 mL)/water (4 mL) and in the same manner as in Example C-72(ii), the title compound (809 mg) was obtained as a black-brown oil.

¹H-NMR (CDCl₃) δ: 0.26-0.38 (2H, m), 0.55-0.68 (2H, m), 1.13-1.34 (1H, m), 2.09 (3H, s), 3.54 (2H, br s), 3.73 (2H, d, J=6.9 Hz), 6.37-6.45 (2H, m), 6.47-6.54 (2H, m), 6.57 (1H, d, J=2.5 Hz), 6.78. (1H, d, J=8.5 Hz), 7.07-7.16 (1H, m).

### (iv) Production of 2-[4-({4-[3-(cyclopropylmethoxy)phenoxy]-3-methylphenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (99.7 mg), 4-[3-(cyclopropylmethoxy)phenoxy]-3-methylaniline (98.7 mg), isopropyl alcohol (3 mL) and 1N aqueous sodium hydroxide solution (1.5 mL) and in the same manner as in Example C-72(iii), the title compound (106 mg) was obtained as white crystals.

¹H-NMR (CDCl₃) δ: 0.29-0.39 (2H, m), 0.58-0.70 (2H, m), 1.17-1.33 (1H, m), 2.24 (3H, s), 3.77 (2H, d, J=7.0 Hz), 4.08-4.17 (2H, m), 4.37 (2H, t, J=4.4 Hz), 6.13 (1H, d, J=3.0 Hz), 6.47-6.62 (3H, m), 6.94 (1H, d, J=3.0 Hz), 6.97 (1H, d, J=8.5 Hz), 7.17 (1H, t, J=8.1 Hz), 7.37-7.48 (2H, m), 8.23 (1H, s), 9.28 (1H, s).

### Example C-109

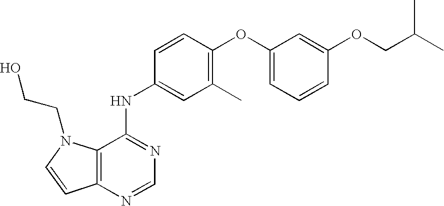

### Production of 2-(4-{[4-(3-isobutoxyphenoxy)-3-methylphenyl]amino}-5H-pyrrolo[3,2-d]pyrimidin-5-yl)ethanol

### (i) Production of 1-(3-isobutoxyphenoxy)-2-methyl-4-nitrobenzene

Using 3-(2-methyl-4-nitrophenoxy)phenol (822 mg), isobutyl bromide (0.55 mL), potassium carbonate (834 mg) sand N,N-dimethylformamide (10 mL) and in the same manner as in Example C-104(i), the title compound (884 mg) was obtained as a pale-yellow oil.

¹H-NMR (CDCl₃) δ: 1.02 (6H, d, J=6.8 Hz), 1.97-2.18 (1H, m), 2.40 (3H, s), 3.70 (2H, d, J=6.4 Hz), 6.52-6.64 (2H, m), 6.71-6.78 (1H, m), 6.82 (1H, d, J=9.1 Hz), 7.22-7.33. (1H, m), 7.99 (1H, dd, J=3.0 Hz, 9.1 Hz), 8.14 (1H, d, J=3.0 Hz).

### (ii) Production of 4-(3-isobutoxyphenoxy)-3-methylaniline

Using 1-(3-isobutoxyphenoxy)-2-methyl-4-nitrobenzene (879 mg), reduced iron (823 mg), calcium chloride (162 mg) and ethanol (27 mL)/water (3 mL) and in the same manner as in Example C-72(ii), the title compound (756 mg) was obtained as a brown oil.

¹H-NMR (CDCl₃) δ: 1.00 (6H, d, J=6.6 Hz), 1.95-2.10 (1H, m), 2.11 (3H, s), 3.55 (2H, br s), 3.66 (2H, d, J=6.3 Hz), 6.35-6.44 (2H, m), 6.49-6.52 (1H, m), 6.52-6.55 (1H, m), 6.58 (1H, d, J=3.0 Hz), 6.80 (1H, d, J=8.5 Hz), 7.12 (1H, t, J=8.1 Hz).

### (iii) Production of 2-(4-{[4-(3-isobutoxyphenoxy)-3-methylphenyl]amino}-5H-pyrrolo[3,2-d]pyrimidin-5-yl)ethanol

Using 2-(4-chloro-5H-pyrrolo[3,2-d]pyrimidin-5-yl)ethyl benzoate (110 mg), 4-(3-isobutoxyphenoxy)-3-methylaniline (103 mg), isopropyl alcohol (3 mL) and 1N aqueous sodium hydroxide solution (1.5 mL) and in the same manner as in Example C-72(iii), the title compound (116 mg) was obtained as white crystals.

¹H-NMR (CDCl₃) δ: 1.02 (6H, d, J=6.8 Hz), 1.97-2.16 (1H, m), 2.25 (3H, s), 3.70 (2H, d, J=6.4 Hz), 4.12 (2H, t, J=4.3 Hz), 4.35 (2H, t, J=4.3 Hz), 6.10 (1H, d, J=3.2 Hz), 6.45-6.54 (2H, m), 6.54-6.62 (1H, m), 6.91 (1H, d, J=3.2 Hz), 6.97 (1H, d, J=8.5 Hz), 7.17 (1H, t, J=8.3 Hz), 7.37-7.48 (2H, m), 8.21 (1H, s), 9.31 (1H, s).

### Example C-110

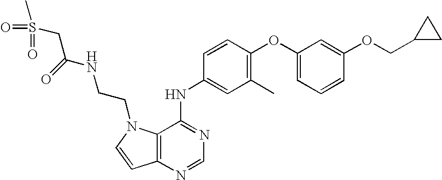

### Production of N-{2-[4-({4-[3-(cyclopropylmethoxy)phenoxy]-3-methylphenyl}amino)-5H-pyrrolo[3,2-d]pyrimidin-5-yl]ethyl}-2-(methylsulfonyl)acetamide

### (i) Production of tert-butyl{2-[4-({4-[3-(cyclopropylmethoxy)phenoxy]-3-methylphenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (402 mg), 4-[3-(cyclopropylmethoxy)phenoxy]-3-methylaniline (395 mg) and isopropyl alcohol (15 mL) and in the same manner as in Example C-105(i), the title compound (710 mg) was obtained as a pale-orange powder.

¹H-NMR (CDCl₃) δ: 0.27-0.39 (2H, m), 0.54-0.69 (2H, m), 1.16-1.32 (1H, m), 1.46 (9H, s), 2.22 (3H, s), 3.41-3.58 (2H, m), 3.76 (2H, d, J=6.8 Hz), 4.39-4.53 (2H, m), 4.99 (1H, d, J=9.5 Hz), 6.46-6.63 (4H, m), 6.95 (1H, d, J=9.5 Hz), 7.09-7.20 (2H, m), 7.55-7.73 (2H, m), 8.28 (1H, br s), 8.49 (1H, s).

### (ii) Production of 5-(2-aminoethyl)-N-{4-[3-(cyclopropylmethoxy)phenoxy]-3-methylphenyl}-5H-pyrrolo[3,2-d]pyrimidin-4-amine dihydrochloride

Using tert-butyl {2-[4-({4-[3-(cyclopropylmethoxy)phenoxy]-3-methylphenyl}amino)-5H-pyrrolo[3,2-d]pyrimidin-5-yl]ethyl}carbamate (707 mg), 6N hydrochloric acid (1 mL) and ethanol (3 mL) and in the same manner as in Example C-105(ii), the title compound (578 mg) was obtained as a yellow powder.

¹H-NMR (DMSO-d₆) δ: 0.25-0.36 (2H, m), 0.49-0.62 (2H, m), 1.12-1.27 (1H, m), 2.19 (3H, s), 3.20-3.33 (2H, m), 3.79 (2H, d, J=6.9 Hz), 5.01 (2H, t, J=6.1 Hz), 6.40-6.49 (2H, m) 6.62-6.69 (1H, m), 6.71 (1H, d, J=3.0 Hz), 7.01 (1H, d, J=8.5 Hz), 7.19-7.29-(1H, m), 7.40 (1H, dd, J=2.5 Hz, 8.5 Hz), 7.49 (1H, d, J=2.5 Hz), 8.04 (1H, d, J=3.0 Hz), 8.33 (3H, br s), 8.67 (1H, s), 9.90 (1H, br s).

### (iii) Production of N-{2-[4-({4-[3-(cyclopropylmethoxy)phenoxy]-3-methylphenyl}amino)-5H-pyrrolo[3,2-d]pyrimidin-5-yl]ethyl}-2-(methylsulfonyl)acetamide

Using 5-(2-aminoethyl)-N-{4-[3-(cyclopropylmethoxy)phenoxy]-3-methylphenyl}-5H-pyrrolo[3,2-d]pyrimidin-4-amine dihydrochloride (120 mg), methylsulfonylacetic acid (50.5 mg), tetrahydrofuran (0.6 mL)/N,N-dimethylformamide (0.6 mL), 1-hydroxybenzotriazole (51.5 mg), triethylamine (0.35 mL) and 1-ethyl-3-(3-dimethylaminopropyl)carbodiimide hydrochloride (116 mg) and in the same manner as in Example C-72(i), the title compound (72.4 mg) was obtained as a pale-yellow powder.

¹H-NMR (CDCl₃) δ: 0.28-0.39 (2H, m), 0.57-0.70 (2H, m), 1.17-1.34 (1H, m), 2.24 (3H, s), 3.08 (3H, s), 3.62-3.75 (2H, m), 3.77 (2H, d, J=7.0 Hz), 3.93 (2H, s), 4.38-4.53 (2H, m), 6.45-6.63 (4H, m), 6.95 (1H, d, J=8.5 Hz), 7.11-7.23 (2H, m), 7.36-7.50 (2H, m), 7.57 (1H, d, J=2.1 Hz), 7.83 (1H, s), 8.47 (1H, s).

### Example C-111

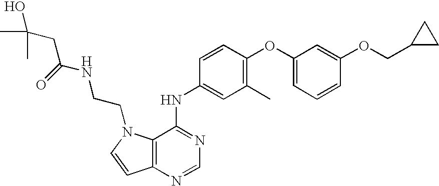

### Production of N-{2-[4-({4-[3-(cyclopropylmethoxy)phenoxy]-3-methylphenyl}amino)-5H-pyrrolo[3,2-d]pyrimidin-5-yl]ethyl}-3-hydroxy-3-methylbutanamide

Using 5-(2-aminoethyl)-N-{4-[3-(cyclopropylmethoxy)phenoxy]-3-methylphenyl}-5H-pyrrolo[3,2-d]pyrimidin-4-amine dihydrochloride (121 mg), 3-hydroxy-3-methylbutanoic acid (46.2 mg), tetrahydrofuran (0.6 mL)/N,N-dimethylformamide (0.6 mL), 1-hydroxybenzotriazole (59.8 mg), triethylamine (0.35 mL) and 1-ethyl-3-(3-dimethylaminopropyl)carbodiimide hydrochloride (88.0 mg) and in the same manner as in Example C-72(i), the title compound (60.0 mg) was obtained as a white powder.

¹H-NMR (CDCl₃) δ: 0.29-0.38. (2H, m), 0.58-0.68 (2H, m), 1.17-1.33 (1H, m), 1.30 (6H, s), 2.24 (3H, s), 2.44 (2H, s), 3.56-3.70 (2H, m), 3.77 (2H, d, J=7.0 Hz), 4.41-4.53 (2H, m), 6.48-6.62 (4H, m), 6.86-6.93 (1H, m), 6.95 (1H, d, J=8.7 Hz), 7.11-7.22 (2H, m), 7.54 (1H, dd, J=2.5 Hz, 8.7 Hz), 7.62 (1H, d, J=2.5 Hz), 8.32 (1H, s), 8.47 (1H, s).

### Example C-112

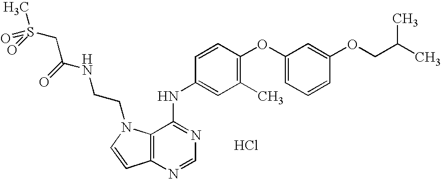

Production of N-[2-(4-{[4-(3-isobutoxyphenoxy)-3-methylphenyl]amino}-5H-pyrrolo[3,2-d]pyrimidin-5-yl)ethyl]-2-(methylsulfonyl)acetamide hydrochloride

### (i) Production of tert-butyl[2-(4-{[4-(3-isobutoxyphenoxy)-3-methylphenyl]amino}-5H-pyrrolo[3,2-d]pyrimidin-5-yl)ethyl]carbamate

Using tert-butyl[2-(4-chloro-5H-pyrrolo[3,2-d]pyrimidin-5-yl)ethyl]carbamate (307 mg), 4-(3-isobutoxyphenoxy)-3-methylaniline (304 mg) and isopropyl alcohol (12 mL) and in the same manner as in Example C-105(i), the title compound (504 mg) was obtained as a white powder.

¹H-NMR (CDCl₃) δ: 1.01 (6H, d, J=6.8 Hz), 1.46 (9H, s), 1.97-2.14 (1H, m), 2.23 (3H, s), 3.41-3.58 (2H, m), 3.68 (2H, d, J=6.4Hz), 4.39-4.54 (2H, m), 5.01 (1H, t, J=5.5 Hz), 6.45-6.63 (4H, m), 6.96 (1H, d, J=8.7 Hz), 7.09-7.21 (2H, m), 7.58-7.71 (2H, m), 8.32 (1H, br s), 8.49 (1H, s).

### (ii) Production of 5-(2-aminoethyl)-N-[4-(3-isobutoxyphenoxy)-3-methylphenyl]-5H-pyrrolo[3,2-d]pyrimidin-4-amine dihydrochloride

Using tert-butyl[2-(4-{[4-(3-isobutoxyphenoxy)-3-methylphenyl]amino}-5H-pyrrolo[3,2-d]pyrimidin-5-yl)ethyl]carbamate (499 mg), 6N hydrochloric acid (1 mL) and ethanol (3 mL) and in the same manner as in Example C-105(ii), the title compound (429 mg) was obtained as a yellow powder.

¹H-NMR (DMSO-d₆) δ: 0.96 (6H, d, J=6.6 Hz), 1.91-2.08 (1H, m), 2.20 (3H, s), 3.21-3.85 (2H, m), 3.73 (2H, d J=6.4 Hz), 5.02 (2H, t, J=6.1 Hz), 6.40-6.52 (2H, m), 6.64-6.70 (1H, m), 6.72 (1H, d, J=3.2 Hz), 7.02 (1H, d, J=8.7 Hz), 7.26 (1H, t, J=8.2 Hz), 7.41 (1H, dd, J=2.5 Hz, 8.7 Hz), 7.50 (1H, d, J=2.5 Hz), 8.05 (1H, d, J=3.2 Hz), 8.37 (3H, br s), 8.68 (1H, s), 9.94 (1H, br s).

### (iii) Production of N-[2-(4-{[4-(3-isobutoxyphenoxy)-3-methylphenyl]amino}-5H-pyrrolo[3,2-d]pyrimidin-5-yl)ethyl]-2-(methylsulfonyl)acetamide hydrochloride

Using 5-(2-aminoethyl)-N-[4-'(3-isobutoxyphenoxy)-3-methylphenyl]-5H-pyrrolo[3,2-d]pyrimidin-4-amine dihydrochloride (119 mg), methylsulfonylacetic acid (50.3 mg), tetrahydrofuran (0.6 mL)/N,N-dimethylformamide (0.6 mL), 1-hydroxybenzotriazole (50.1 mg), triethylamine (0.35 mL) and 1-ethyl-3-(3-dimethylaminopropyl)carbodiimide hydrochloride (82.7 mg) and in the same manner as in Example C-72(i), N-[2-(4-{[4-(3-isobutoxyphenoxy)-3-methylphenyl]amino}-5H-pyrrolo[3,2-d]pyrimidin-5-yl)ethyl]-2-(methylsulfonyl)acetamide was obtained. The compound was dissolved in ethyl acetate and treated with 4N hydrogen chloride/ethyl acetate solution, and the precipitate was collected by filtration to give the title compound (104 mg) as white crystals.

¹H-NMR (DMSO-d₆) δ: 0.96 (6H, d, J=6.6 Hz), 1.88-2.09 (1H, m), 2.20 (3H, s), 3.06 (3H, s), 3.54 (2H, q, J=6.0 Hz), 3.73 (2H, d, J=6.6 Hz), 4.06 (2H, s), 4.69 (2H, t, J=6.0 Hz), 6.39-6.52 (2H, m), 6.64 (1H, d, J=3.2 Hz), 6.65-6.72 (1H, m), 7.02 (1H, d, J=8.8 Hz), 7.26 (1H, t, J=8.2 Hz), 7.45 (1H, dd, J=2.5 Hz, 8.8 Hz), 7.52 (1H, d, J=2.5 Hz), 7.90 (1H, d, J=3.2 Hz), 8.67 (1H, s), 8.77 (1H, t, J=6.0 Hz), 9.82 (1H, s).

### Example C-113

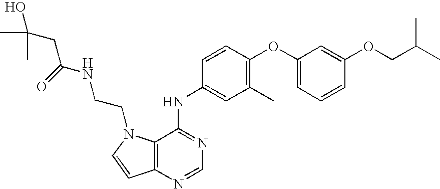

### Production of 3-hydroxy-N-[2-(4-{[4-(3-isobutoxyphenoxy)-3-methylphenyl]amino}-5H-pyrrolo[3,2-d]pyrimidin-5-yl)ethyl]-3-methylbutanamide

Using 5-(2-aminoethyl)-N-[4-(3-isobutoxyphenoxy)-3-methylphenyl]-5H-pyrrolo[3,2-d]pyrimidin-4-amine dihydrochloride (120 mg), 3-hydroxy-3-methylbutanoic acid (46.1 mg), tetrahydrofuran (0.6 mL)/N,N-dimethylformamide (0.6 mL), 1-hydroxybenzotriazole (50.2 mg), triethylamine (0.35 mL) and 1-ethyl-3-(3-dimethylaminopropyl)carbodiimide hydrochloride. (82.7 mg) and in the same manner as in Example C-72(i), the title compound (91.0 mg) was obtained as white crystals.

¹H-NMR (CDCl₃) δ: 1.01 (6H, d, J=6.6 Hz), 1.30 (6H, s), 1.97-2.15 (1H, m), 2.25 (3H, s), 2.44 (2H, s), 3.57-3.67 (2H, m), 3.69 (2H, d, J=6.6 Hz), 4.41-4.54 (2H, m), 6.44-6.63 (4H, m), 6.84 (1H, t, J=5.9 Hz), 6.95 (1H, d, J=8.7 Hz), 7.11-7.21 (2H, m), 7.55 (1H, dd, J=2.5 Hz, 8.7 Hz), 7.63 (1H, d, J=2.5 Hz), 8.31 (1H, s), 8.48 (1H, s).

### Example C-114

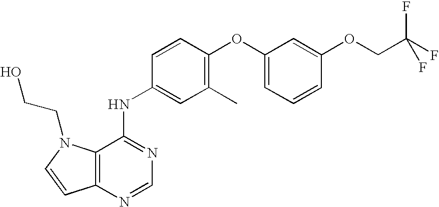

### Production of 2-[4-({3-methyl-4-[3-(2,2,2-trifluoroethoxy)phenoxy]phenyl}amino)-5H-pyrrolo[3,2-d]pyrimidin-5-yl]ethanol

### (i) Production of 2-methyl-4-nitro-1-[3-(2,2,2-trifluoroethoxy)phenoxy]benzene

Using 3-(2-methyl-4-nitrophenoxy)phenol (797 mg), 1,1,1-trifluoro-2-iodoethane (0.5 mL), potassium carbonate (903 mg) and N,N-dimethylformamide (10 mL), and in the same manner as in Example C-104(i), the title compound (780 mg) was obtained as a yellow oil.

¹H-NMR (CDCl₃) δ: 2.39 (3H, s), 4.35 (2H, q, J=8.0 Hz), 6.64 (1H, t, J=2.4 Hz), 6.67-6.74-(1H, m), 6.74-6.81 (1H, m), 6.84 (1H, d, J=9.0 Hz), 7.34 (1H, t, J=8.3 Hz), 8.02 (1H, dd, J=2.8 Hz, 9.0 Hz), 8;16 (1H, d, J=2.8 Hz).

### (ii) Production of 3-methyl-4-[3-(2,2,2-trifluoroethoxy)phenoxy]aniline

Using 2-methyl-4-nitro-1-[3-(2,2,2-trifluoroethoxy)phenoxy]benzene (774 mg), reduced iron (685 mg), calcium chloride (138 mg) and ethanol (22.5 mL)/water (2.5 mL) and in the same manner as in Example C-72(ii), the title compound (529 mg) was obtained as a white powder.

¹H-NMR (CDCl₃) δ: 2.08 (3H, s), 3.57 (2H, br s), 4.28 (2H, q, J=8.0 Hz), 6.43 (1H, t, J=2.5 Hz), 6.48-6.56 (3H, m), 6.58 (1H, d, J=2.8 Hz), 6.78 (1H, d, J=8.5 Hz), 7.17 (1H, t, J=8.3 Hz).

### (iii) Production of 2-[4-({3-methyl-4-[3-(2,2,2-trifluoroethoxy)phenoxy]phenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (101 mg), 3-methyl-4-[3-(2,2,2-trifluoroethoxy)phenoxy]aniline (100 mg), isopropyl alcohol (3 mL) and 1N aqueous sodium hydroxide solution (1 mL) and in the same manner as in Example C-72(iii), the title compound (63.9 mg) was obtained as a white powder.

¹H-NMR (CDCl₃) δ: 2.23 (3H, s), 4.13 (2H, t, J=4.4 Hz), 4.25-4.43 (4H, m), 6.13 (1H, d, J=3.2 Hz), 6.54 (1H, t, J=2.3 Hz), 6.56-6.68 (2H, m), 6.94 (1H, d, J=3.2 Hz), 6.98 (1H, d, J=8.5 Hz), 7.23 (1H, t, J=8.3 Hz), 7.39-7.49 (2H, m), 8.23 (1H, s), 9.32 (1H, s).

### Example C-115

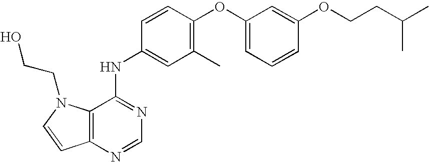

### Production of 2-[4-({3-methyl-4-[3-(3-methylbutoxy)phenoxy]phenyl}amino)-5H-pyrrolo[3,2-d]pyrimidin-5-yl]ethanol

### (i) Production of 2-methyl-1-[3-(3-methylbutoxy)phenoxy]-4-nitrobenzene

Using 3-(2-methyl-4-nitrophenoxy)phenol (855 mg), 1-iodo-3-methylbutane (0.8 mL), potassium carbonate (968 mg) and N,N-dimethylformamide (10 mL) and in the same, manner as in Example C-104(i), the title compound (983 mg) was obtained as a yellow oil.

¹H-NMR (CDCl₃) δ: 0.96 (6H, d, J=6.6 Hz), 1.67 (2H, q, J=6.6 Hz), 1.74-1.93 (1H, m), 2.40 (3H, s), 3.97 (2H, t, J=6.6 Hz), 6.55-6.62 (2H, m), 6.71-6.79 (1H, m), 6.82 (1H, d, J=9.0 Hz), 7.24-7.33 (1H, m), 8.00 (1H, dd, J=2.6 Hz, 9.0 Hz), 8.15 (1H, d, J=2.6 Hz).

### (ii) Production of 3-methyl-4-[3-(3-methylbutoxy)phenoxy]aniline

Using 2-methyl-1-[3-(3-methylbutoxy)phenoxy]-4-nitrobenzene (978 mg), reduced iron (879 mg), calcium chloride (176 mg) and ethanol (27 mL)/water (3 mL) and in the same manner as in Example C-72(ii), the title compound (835 mg) was obtained as a brown oil.

¹H-NMR (CDCl₃) δ: 0.94 (6H, d, J=6.7 Hz), 1.64 (2H, q, J=6.7 Hz), 1.72-1.90 (1H, m), 2.10 (3H, s), 3.55 (2H, br s), 3.93 (2H, t, J=6.7 Hz), 6.36-6.44 (2H, m), 6.48-6.55 (2H, m), 6.58 (1H, d, J=2.8 Hz), 6.80 (1H, d, J=8.3 Hz), 7.06-7.18 (1H, m).

### (iii) Production of 2-[4-({3-methyl-4-[3-(3-methylbutoxy)phenoxy]phenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (105 mg), 3-methyl-4-[3-(3-methylbutoxy)phenoxy]aniline (102′mg), isopropyl alcohol (3 mL) and 1N aqueous sodium hydroxide solution (1 mL) and in the same manner as in Example C-72(iii), the title compound (91.5 mg) was obtained as white crystals.

¹H-NMR (CDCl₃) δ: 0.96 (6H, d, J=6.7 Hz), 1.66 (2H, q, J=6.7 Hz), 1.75-1.92 (1H, m), 2.25 (3H, s), 3.96 (2H, t, J=6.7 Hz), 4.12 (2H, t, J=4.4 Hz), 4.31-4.41 (2H, m), 6.11 (1H, d, J=3.2 Hz), 6.46-6.54 (2H, m), 6.54-6.62 (1H, m), 6.92 (1H, d, J=3.2 Hz), 6.97 (1H, d, J=8.3 Hz), 7.12-7.22 (1H, m), 7.36-7.50 (2H, m), 8.22 (1H, s), 9.29 (1H, s).

### Example C-116

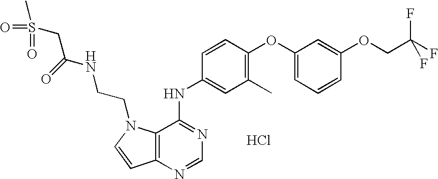

### Production of 2-(methylsulfonyl)-N-{2-[4-({3-methyl-4-[3-(2,2,2-trifluoroethoxy)phenoxy]phenyl}amino)-5H-pyrrolo[3,2-d]pyrimidin-5-yl]ethyl}acetamide hydrochloride

### (i) Production of tert-butyl{2-[4-({3-methyl-4-[3-(2,2,2-trifluoroethoxy)phenoxy]phenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (305 mg), 3-methyl-4-[3-(2,2,2-trifluoroethoxy)phenoxy]aniline (326 mg) and isopropyl alcohol (12 mL) and in the same manner as in Example C-105(i), the title compound (489 mg) was obtained as a white powder.

¹H-NMR (CDCl₃) δ: 1.46 (9H, s), 2.21 (3H, s), 3.42-3.58 (2H, m), 4.31 (2H, q, J=8.1 Hz), 4.41-4.55 (2H, m), 5.03 (1H, t, J=5.0 Hz), 6.53 (1H, t, J=2.4 Hz), 6.56-6.67 (3H, m), 6.91-7.02 (1H, m), 7.16 (1H, d, J=3.2 Hz), 7.21 (1H, t, J=8.2 Hz), 7.56-7.77 (2H, m), 8.38 (1H, br s), 8.49 (1H, s).

### (ii) Production of 5-(2-aminoethyl)-N-{3-methyl-4-[3-(2,2,2-trifluoroethoxy)phenoxy]phenyl}-5H-pyrrolo[3,2-d]pyrimidin-4-amine dihydrochloride

Using tert-butyl{2-[4-({3-methyl-4-[3-(2,2,2-trifluoroethoxy)phenoxy]phenyl}amino)-5H-pyrrolo[3,2-d]pyrimidin-5-yl]ethyl}carbamate (484 mg), 6N hydrochloric acid (1 mL) and ethanol (3 mL) and in the same manner as in Example C-105(ii), the title compound (423 mg) was obtained as a yellow powder.

¹H-NMR (DMSO-d₆) δ: 2.20 (3H, s), 3.21-3.33 (2H, m), 4.78 (2H, q, J=8.9 Hz), 5.01 (2H, t, J=6.1 Hz), 6.56 (1H, dd, J=2.0 Hz, 8.0 Hz), 6.64 (1H, t, J=2.0 Hz), 6.72 (1H, d, J=3.2 Hz), 6.81 (1H, dd, J=2.0 Hz, 8.0 Hz), 7.04 (1H, d, J=8.7 Hz), 7.33 (1H, t, J=8.0 Hz), 7.43 (1H, dd, J=2.5 Hz, 8.7 Hz), 7.52 (1H, d, J=2.5 Hz), 8.04 (1H, d, J=3.2 Hz), 8.34 (3H, br s), 8.68 (1H, s), 9.92 (1H, br s).

### (iii) Production of 2-(methylsulfonyl)-N-{2-[4-({3-methyl-4-[3-(2,2,2-trifluoroethoxy)phenoxy]phenyl}amino)-5H-pyrrolo[3,2-d]pyrimidin-5-yl]ethyl}acetamide hydrochloride

Using 5-(2-aminoethyl)-N-{3-methyl-4-[3-(2,2,2-trifluoroethoxy)phenoxy]phenyl}-5H-pyrrolo[3,2-d]pyrimidin-4-amine dihydrochloride (152 mg), methylsulfonylacetic acid (64.5 mg), tetrahydrofuran (0.8 mL)/N,N-dimethylformamide (0.8 mL), 1-hydroxybenzotriazole (70.6 mg), triethylamine (0.4 mL) and 1-ethyl-3-(3-dimethylaminopropyl)carbodiimide hydrochloride (93.8 mg) and in the same manner as in Example C-72(i), 2-(methylsulfonyl)-N-{2-[4-({3-methyl-4-[3-(2,2,2-trifluoroethoxy)phenoxy]phenyl}amino)-5H-pyrrolo[3,2-d]pyrimidin-5-yl]ethyl}acetamide was obtained. The compound was dissolved in ethyl acetate and treated with 4N hydrogen chloride/ethyl acetate solution, and the precipitate was collected by filtration to give the title compound (132 mg) as a pale-yellow powder.

¹H-NMR (DMSO-d₆) δ: 2.20 (3H, s), 3.05 (3H, s), 3.49-3.60 (2H, m), 4.07 (2H, s), 4.69 (2H, t, J=6.5 Hz), 4.77 (2H, q, J=8.9 Hz), 6.52-6.59 (1H, m), 6.61-6.68 (2H, m), 6.81 (1H, dd, J=2.3 Hz, 8.1 Hz), 7.04 (1H, d, J=8.7 Hz), 7.33 (1H, t, J=8.1 Hz), 7.47 (1H, dd, J=2.5 Hz, 8.7 Hz), 7.54 (1H, d, J=2.5 Hz), 7.91 (1H, d, J=3.0 Hz), 8.67 (1H, s), 8.79 (1H, t, J=5.5 Hz), 9.85 (1H, s).

### Example C-117

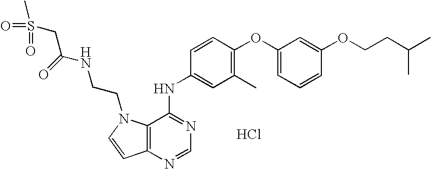

### Production of N-{2-[4-({3-methyl-4-[3-(3-methylbutoxy)phenoxy]phenyl}amino)-5H-pyrrolo[3,2-d]pyrimidin-5-yl]ethyl}-2-(methylsulfonyl)acetamide hydrochloride

### (i) Production of tert-butyl{2-[4-({3-methyl-4-[3-(3-methylbutoxy)phenoxy]phenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (400 mg), 3-methyl-4-[3-(3-methylbutoxy)phenoxy]aniline (408 mg) and isopropyl alcohol (15 mL) and in the same manner as in Example C-105(i); the title compound (692 mg) was obtained as a white powder.

¹H-NMR (CDCl₃) δ: 0.95 (6H, d, J=6.6 Hz), 1.46 (9H, s), 1.61-1.71 (2H, m), 1.74-1.91 (1H, m), 2.23 (3H, s), 3.42-3.58 (2H, m), 3.95 (2H, t, J=6.6 Hz), 4.39-4.54 (2H, m), 5.01 (1H, t, J=6.1 Hz), 6.45-6.62 (4H, m), 6.96 (1H, d, J=8.9 Hz), 7.10-7.22 (2H, m), 7.57-7.74 (2H, m), 8.33 (1H, br s), 8.49 (1H, s).

### (ii) Production of 5-(2-aminoethyl)-N-{3-methyl-4-[3-(3-methylbutoxy)phenoxy]phenyl}-5H-Pyrrolo[3,2-d]pyrimidin-4-amine dihydrochloride

Using tert-butyl{2-[4-({3-methyl-4-[3-(3-methylbutoxy)phenoxy]phenyl}amino)-5H-pyrrolo[3,2-d_{]pyrimidin-}5-yl]ethyl}carbamate (685 mg), 6N hydrochloric acid (1 mL) and ethanol (3 mL) and in the same manner as in Example C-105(ii), the title compound (602 mg) was obtained as a yellow powder.

¹H-NMR (DMSO-d₆) δ: 0.92 (6H, d, J=6.6 Hz), 1.59 (2H, q, J=6.6 Hz), 1.67-1.86 (1H, m), 2.20 (3H, s), 3.20-3.33 (2H, m), 3.97 (2H, t, J=6.6 Hz), 5.01 (2H, t, J=6.2 Hz), 6.40-6.52 (2H, m), 6.68 (1H, dd, J=2.0 Hz, 8.3 Hz), 6.72 (1H, d, J=3.0 Hz), 7.02 (1H, d, J=8.7 Hz), 7.26 (1H, t, J=8.3 Hz), 7.41 (1H, dd, J=2.5 Hz, 8.7 Hz), 7.50 (1H, d, J=2.0 Hz), 8.05 (1H, d, J=3.0 Hz), 8.35 (3H, br s), 8.67 (1H, s), 9.91 (1H, br s).

### (iii) Production of N-{2-[4-({3-methyl-4-[3-(3-methylbutoxy)phenoxy]phenyl}amino)-5H-pyrrolo[3,2-d]pyrimidin-5-yl]ethyl}-2-(methylsulfonyl)acetamide hydrochloride

Using 5-(2-aminoethyl)-N-{3-methyl-4-[3-(3-methylbutoxy)phenoxy]phenyl}-5H-pyrrolo[3,2-d]pyrimidin-4-amine dihydrochloride (1.50 mg), methylsulfonylacetic acid (60.9 mg), tetrahydrofuran (0.8 mL)/N,N-dimethylformamide (0.8 mL), 1-hydroxybenzotriazole (68.1 mg), triethylamine (0.4 mL) and 1-ethyl-3-(3-dimethylaminopropyl)carbodiimide hydrochloride (92.2 mg) and in the same manner as in Example C-72(i), N-{2-[4-({3-methyl-4-[3-(3-methylbutoxy)phenoxy]phenyl}amino)-5H-pyrrolo[3,2-d]pyrimidin-5-yl]ethyl}-2-(methylsulfonyl)acetamide was obtained. The compound was dissolved in ethyl acetate and treated with 4N-hydrogen chloride/ethyl acetate solution, and the precipitate was collected by filtration to give the title compound (116 mg) as a pale-yellow powder.

¹H-NMR (DMSO-d₆) δ: 0.92 (6H, d, J=6.6 Hz), 1.59 (2H, q, J=6.6 Hz), 1.67-1.84 (1H, m), 2.20 (3H, s), 3.05 (3H, s), 3.54 (2H, q, J=6.0 Hz), 3.97 (2H, t, J=6.6 Hz), 4.06 (2H, s), 4.69 (2H, t, J=6.0 Hz), 6.40-6.52 (2H, m), 6.64 (1H, d, J=3.2 Hz), 6.66-6.72 (1H, m), 7.02 (1H, d, J=8.7 Hz), 7.26 (1H, t, J=8.2 Hz), 7.45 (1H, dd, J=2.5 Hz, 8.7 Hz), 7.52 (1H, d, J=2.5 Hz), 7.90 (1H, d, J=3.2 Hz), 8.67 (1H, s), 8.78 (1H, t, J=6.0 Hz), 9.83 (1H, s).

### Example C-118

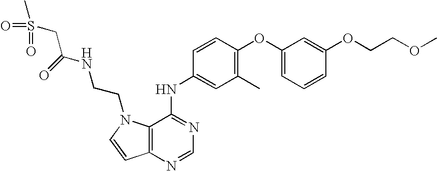

### Production of N-{2-[4-({4-[3-(2-methoxyethoxy)phenoxy]-3-methylphenyl}amino)-5H-pyrrolo[3,2-d]pyrimidin-5-yl]ethyl}-2-(methylsulfonyl)acetamide

### (i) Production of 1-[3-(2-methoxyethoxy)phenoxy]-2-methyl-4-nitrobenzene

Using 3-(2-methyl-4-nitrophenoxy)phenol (806 mg), 1-bromo-2-methoxyethane (0.5 mL), potassium carbonate (910 mg) and N,N-dimethylformamide (10 mL) and in the same manner as in Example C-104(i), the title compound (855 mg) was obtained as a,yellow oil.

¹H-NMR (CDCl₃) δ: 2.39 (3H, s), 3.45 (3H, s), 3.69-3.80 (2H, m), 4.06-4.15 (2H, m), 6.57-6.65 (2H, m), 6.75-6.80 (1H, m), 6.82 (1H, d, J=9.0 Hz),7.24-7.33 (1H, m), 7.99 (1H, dd, J=2.8 Hz, 9.0 Hz), 8.15 (1H, d, J=2.8 Hz).

### (ii) Production of 4-[3-(2-methoxyethoxy)phenoxy]-3-methylaniline

Using 1-[3-(2-methoxyethoxy)phenoxy]-2-methyl-4-nitrobenzene (850 mg), reduced iron (825 mg), calcium chloride (167 mg) and ethanol (27 mL)/water (3 mL) and in the same manner as in Example C-72(ii), the title compound (707 mg) was obtained as a brown oil.

¹H-NMR (CDCl₃) δ: 2.09 (3H, s), 3.43 (3H, s), 3.56 (2H, br s), 3.67-3.76 (2H, m), 4.02-4.09 (2H, m), 6.35-6.63 (5H, m), 6.79 (1H, d, J=8.3 Hz), 7.13 (1H, t, J=8.3 Hz)

### (iii) Production of tert-butyl{2-[4-({4-[3-(2-methoxyethoxy)phenoxy]-3-methylphenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (358 mg), 4-[3-(2-methoxyethoxy)phenoxy]-3-methylaniline (364 mg) and isopropyl alcohol (15 mL) and in the same manner as in Example C-105(i), the title compound (563 mg) was obtained as a white powder.

¹H-NMR (CDCl₃) δ: 1.46 (9H, s), 2.22 (3H, s),'3.44 (3H, s), 3.45-3.56 (2H, m), 3.69-3.77 (2H, m), 4.04-4.10 (2H, m), 4.40-4.52 (2H, m), 5.01 (1H, t, J=5.7 Hz), 6.49-6.65 (4H, m), 6.91-6.99 (1H, m), 7.12-7.23 (2H, m), 7.53-7.73 (2H, m), 8.31 (1H, br s), 8.49 (1H, s).

### (iv) Production of 5-(2-aminoethyl)-N-{4-[3-(2-methoxyethoxy)phenoxy]-3-methylphenyl}-5H-pyrrolo[3,2-d]pyrimidin-4-amine dihydrochloride

Using tert-butyl{2-[4-({4-[3-(2-methoxyethoxy)phenoxy]-3-methylphenyl}amino)-5H-pyrrolo[3,2-d]pyrimidin-5-yl]ethyl}carbamate (558 mg), 6N hydrochloric acid (1 mL) and ethanol (4 mL) and in the same manner as in Example C-105(ii), the title compound (471 mg) was obtained as a yellow powder.

¹H-NMR (DMSO-d₆) δ: 2.19 (3H, s), 3.21-3.38 (2H, m), 3.29 (3H, s), 3.58-3.69 (2H, m), 4.00-4.13 (2H, m), 5.00 (2H, t, J=6.0 Hz), 6.44-6.54 (2H, m), 6.65-6.71 (1H, m), 6.72 (1H, d, J=3.2 Hz), 7.03 (1H, d, J=8.7 Hz), 7.20-7.33 (1H, m), 7.41 (1H, dd, J=2.5 Hz, 8.7 Hz), 7.50 (1H, d, J=2.5 Hz), 8.04 (1H, d, J=3.2 Hz), 8.32 (3H, br s), 8.68 (1H, s), 9.88 (1H, br s).

### (v) Production of N-{2-[4-({4-[3-(2-methoxyethoxy)phenoxy]-3-methylphenyl}amino)-5H-pyrrolo[3,2-d]pyrimidin-5-yl]ethyl}-2-(methylsulfonyl)acetamide

Using 5-(2-aminoethyl)-N-{4-[3-(2-methoxyethoxy)phenoxy]-3-methylphenyl}-5H-pyrrolo[3,2-d]pyrimidin-4-amine dihydrochloride (151 mg), methylsulfonylacetic acid (67.3 mg), tetrahydrofuran (0.8 mL)/N,N-dimethylformamide (0.8 mL), 1-hydroxybenzotriazole (94.2 mg), triethylamine (0.4 mL) and 1-ethyl-3-(3-dimethylaminopropyl)carbodiimide hydrochloride (122 mg) and in the same manner as in Example C-72(i), the title compound (98.4 mg) was obtained as a pale-yellow powder.

¹H-NMR (CDCl₃) δ: 2.23 (3H, s), 3.08 (3H, s), 3.44 (3H, s), 3.63-3.78 (4H, m), 3.93 (2H, s), 4.04-4.13 (2H, m), 4.38-4.54 (2H, m), 6.51 (1H, t, J=2.4 Hz), 6.53-6.66 (3H, m), 6.94 (1H, d, J=8.7 Hz), 7.13-7.23 (2H, m), 7.44 (1H, dd, J=2.0 Hz, 8.7 Hz), 7.51 (1H, t, J=5.7 Hz), 7.56 (1H, d, J=2.0 Hz), 7.83 (1H, s), 8.46 (1H, s).

Example C-119

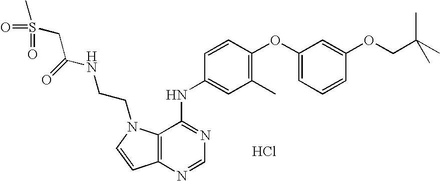

### Production of N-{2-[4-({4-[3-(2,2-dimethylpropoxy)phenoxy]-3-methylphenyl}amino)-5H-pyrrolo[3,2-d]pyrimidin-5-yl]ethyl}-2-(methylsulfonyl)acetamide hydrochloride

### (i) Production of 1-[3-(2,2-dimethylpropoxy)phenoxy]-2-methyl-4-nitrobenzene

Using 3-(2-methyl-4-nitrophenoxy)phenol (803 mg), 1-iodo-2,2-dimethylpropane (0.75 mL), potassium carbonate (911 mg) and N,N-dimethylformamide (10 mL) and in the same manner as in Example C-104(i), the title compound (826 mg) was obtained as a yellow oil.

¹H-NMR (CDCl₃) δ: 1.03 (9H, s), 2.40 (3H, s), 3.57 (2H, s), 6.55-6.62 (2H, m), 6.73-6.79 (1H, m), 6.82 (1H, d, J=9.0 Hz), 7.28 (1H, t, J=8.5 Hz), 8.00 (1H, dd, J=2.5 Hz, 9.0 Hz), 8.15 (1H, d, J=2.5 Hz).

### (ii) Production of 4-[3-(2,2-dimethylpropoxy)phenoxy]-3-methylaniline

Using 1-[3-(2,2-dimethylpropoxy)phenoxy]-2-methyl-4-nitrobenzene (821 mg), reduced iron (876 mg), calcium chloride (147 mg) and ethanol (27 mL)/water (3 mL) and in the same manner as in Example C-72(ii), the title compound (745 mg) was obtained as a brown oil.

¹H-NMR (CDCl₃) δ: 1.01 (9H, s), 2.11 (3H, s), 3.53 (2H, s), 3.54 (2H, br s), 6.35-6.41 (1H, m), 6.43 (1H, t, J=2.4 Hz), 6.48-6.56 (2H, m), 6.59 (1H, d, J=2.8 Hz), 6.80 (1H, d, J=8.0 Hz), 7.12 (1H, t, J=8.0 Hz).

### (iii) Production of tert-butyl{2-[4-({4-[3-(2,2-dimethylpropoxy)phenoxy]-3-methylphenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (351 mg), 4-[3-(2,2-dimethylpropoxy)phenoxy]-3-methylaniline (357 mg) and isopropyl alcohol (15 mL) and in the same manner as in Example C-105(i), the title compound (592 mg) was obtained as a white powder.

¹H-NMR (CDCl₃) δ: 1.02 (9H, s), 1.46 (9H, s), 2.24 (3H, s), 3.42-3.55 (2H, m), 3.56 (2H, s), 4.39-4.55 (2H, m), 5.02 (1H, t, J=5.8 Hz), 6.44-6.65 (4H, m), 6.96 (1H, d, J=8.5 Hz), 7.09-7.22 (2H, m), 7.56-7.72 (2H, m), 8.32 (1H, br s), 8.49 (1H, s).

### (iv) Production of 5-(2-aminoethyl)-N-{4-[3-(2,2-dimethylpropoxy)phenoxy]-3-methylphenyl}-5H-pyrrolo[3,2-d]pyrimidin-4-amine dihydrochloride

Using tert-butyl{2-[4-({4-[3-(2,2-dimethylpropoxy)phenoxy]-3-methylphenyl}amino)-5H-pyrrolo[3,2-d]pyrimidin-5-yl]ethyl}carbamate (586 mg), 6N hydrochloric acid (1 mL) and ethanol (4 mL) and in the same manner as in Example C-105(ii), the title compound (369 mg) was obtained as a yellow powder.

¹H-NMR (DMSO-d₆) δ: 0.98 (9H, s), 2.20 (3H, s), 3.18-3.36 (2H, m), 3.62 (2H, s), 4.99 (2H, t, J=6.2 Hz), 6.40-6.55 (2H, m), 6.63-6.77 (2H, m), 7.02 (1H, d, J=8.5 Hz), 7.26 (1H, t, J=8.2 Hz), 7.41 (1H, dd, J=2.3 Hz, 8.5 Hz), 7.50 (1H, d, J=2.3 Hz), 8.03 (1H, d, J=3.2 Hz), 8.29 (3H, br s), 8.67 (1H, s), 9.86 (1H, br s).

### (v) Production of N-{2-[4-({4-[3-(2,2-dimethylpropoxy)phenoxy]-3-methylphenyl}amino)-5H-pyrrolo[3,2-d]pyrimidin-5-yl]ethyl}-2-(methylsulfonyl)acetamide hydrochloride

Using 5-(2-aminoethyl)-N-{4-[3-(2,2-dimethylpropoxy)phenoxy]-3-methylphenyl}-5H-pyrrolo[3,2-d]pyrimidin-4-amine dihydrochloride (152 mg), methylsulfonylacetic acid (63.0 mg), tetrahydrofuran (0.8 mL)/N,N-dimethylformamide (0.8 mL), 1-hydroxybenzotriazole (86.5 mg), triethylamine (0.4 mL) and 1-ethyl-3-(3-dimethylaminopropyl)carbodiimide hydrochloride (125 mg) and in the same manner as in Example C-72(i), N-{2-[4-({4-[3-(2,2-dimethylpropoxy)phenoxy]-3-methylphenyl}amino)-5H-pyrrolo[3,2-d]pyrimidin-5-yl]ethyl}-2-(methylsulfonyl)acetamide was obtained. The compound was dissolved in ethyl acetate and treated with 4N hydrogen chloride/ethyl acetate solution, and the precipitate was collected by filtration to give the title compound (126 mg) as yellow crystals.

¹H-NMR (DMSO-d₆) δ: 0.98 (9H, s), 2.20 (3H, s), 3.05 (3H, s), 3.54 (2H, q, J=6.0 Hz), 3.61 (2H, s), 4.07 (2H, s), 4.70 (2H, t, J=6.0 Hz), 6.40-6.53 (2H, m), 6.64 (1H, d, J=3.0 Hz), 6.69 (1H, dd, J=1.7 Hz, 8.0 Hz), 7.02 (1H, d, J=8.5 Hz), 7.26 (1H, t, J=8.0 Hz), 7.45 (1H, dd, J=2.0 Hz, 8.5 Hz), 7.53 (1H, d, J=2.0 Hz), 7.91 (1H, d, J=3.0 Hz), 8.67 (1H, s), 8.80 (1H, t, J=6.0 Hz), 9.86 (1H, s).

### Example C-120

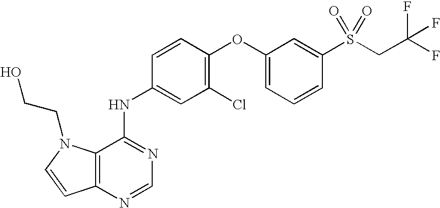

### Production of 2-{4-[(3-chloro-4-{3-[(2,2,2-trifluoroethyl)sulfonyl]phenoxy}phenyl)amino]-5H-pyrrolo[3,2-d]pyrimidin-5-yl}ethanol

### (i) Production of 2-chloro-4-nitro-1-{3-[(2,2,2-trifluoroethyl)thio]phenoxy}benzene

To a solution of 3-mercaptophenol (2.0 g) and triethylamine (2.70 mL) in N,N-dimethylformamide (20 mL) was added 2,2,2-trifluoro-1-iodoethane (1.72 mL) at room temperature. The reaction mixture was stirred at room temperature for 4 hr, 3-chloro-4-fluoronitrobenzene (2.77 g) and potassium carbonate (2.18 g) were added thereto. The reaction mixture was stirred at room temperature for 20 hr, water was added to the reaction mixture and the mixture was extracted with ethyl acetate. The organic layer was washed successively with water and saturated brine, and dried over magnesium sulfate. After concentration under reduced pressure, the residue was separated and purified by column chromatography (eluent, hexane:ethyl acetate=19:1→3:1) to give the title compound (4.27 g) as a yellow oil.

¹H-NMR (CDCl₆) δ: 3.48 (2H, q, J=9.6 Hz), 6.91 (1H, d, J=9.0 Hz), 6.98-7.02 (1H, m), 7.19-7.21 (1H, m), 7.34-7.44 (2H, m), 8.09 (1H, dd, J='2.7 Hz, 9.0 Hz), 8.40 (1H, d, J=2.7 Hz).

### (ii) Production of 2-chloro-4-nitro-1-{3-[(2,2,2-trifluoroethyl)sulfonyl]phenoxy}benzene

To a solution of 2-chloro-4-nitro-1-{3-[(2,2,2-trifluoroethyl)thio]phenoxy}benzene (2.0 g) in ethyl acetate (20 mL) was added 70% 3-chloroperbenzoic acid (2.80 g) at 0° C. The reaction mixture was stirred at 0° C. for 2 hr and at room temperature for 4 days, aqueous sodium thiosulfate solution was added to the reaction mixture, and the mixture was stirred for 1 hr. The mixture was extracted with ethyl acetate, and the organic layer was washed successively with aqueous sodium bicarbonate and saturated brine, and dried over magnesium sulfate. After concentration under reduced pressure, the residue was separated and purified by column chromatography (eluent, hexane:ethyl acetate=9:1→2:3) to give the title compound (2.34 g) as colorless crystals.

¹H-NMR (CDCl₃) δ: 3.94 (2H, q, J=8.7 Hz), 7.01 (1H, d, J=9.0 Hz), 7.38-7.43 (1H, m), 7.60-7.62 (1H, m), 7.66-7.71 (1H, m), 7.81-7.85 (1H, m), 8.15 (1H, dd, J=2.7 Hz, 9.0 Hz), 8.43 (1H, d, J=2.7 Hz).

### (iii) Production of 3-chloro-4-{3-[(2,2,2-trifluoroethyl)sulfonyl]phenoxy}aniline

A mixture of 2-chloro-4-nitro-1-{3-[(2,2,2-trifluoroethyl)sulfonyl]phenoxy}benzene (2.34 g), reduced iron (1.54 g) and calcium chloride (0.31 g) in 15% water-containing ethanol (70 mL) was heated under reflux for 10 hr. The insoluble material was filtered off, and the filtrate was concentrated under reduced pressure. Aqueous sodium bicarbonate was added to the residue, and the mixture was extracted with ethyl acetate. The organic layer was washed with saturated brine and dried over magnesium sulfate. After concentration under reduced pressure, the residue was separated and purified by column chromatography (eluent, hexane:ethyl acetate=3:1→3:7) to give the title compound (892 mg) as a yellow solid.

¹H-NMR (CDCl₃) δ: 3.77 (2H, br s), 3.87 (2H, q, J=9.0 Hz), 6.60 (1H, dd, J=2.7, 8.7 Hz), 6.79 (1H, d, J=2.7 Hz), 6.85 (1H, d, J=8.7 Hz), 7.20-7.25 (1H, m), 7.35-7.37 (1H, m), 7.51 (1H, t, J=8.1 Hz), 7.61 (1H, d, J=8.1 Hz).

### (iv) Production of 2-{4-[(3-chloro-4-{3-[(2,2,2-trifluoroethyl)sulfonyl]phenoxy}phenyl)amino]-5H-pyrrolo[3,2-d]pyrimidin-5-yl}ethanol

A solution of 5-(2-{[tert-butyl(dimethyl)silyl]oxy}ethyl)-4-chloro-5H-pyrrolo[3,2-d]pyrimidine (100 mg) and 3-chloro-4-{3-[(2,2,2-trifluoroethyl)sulfonyl]phenoxy}aniline (117 mg) in isopropyl alcohol (3.0 mL) was stirred at 80° C. for 4 days. After concentration under reduced pressure, methanol (5.0 mL) and 6N hydrochloric acid (1.0 mL) were added to the residue. The mixture was stirred at room temperature for 4 hr, and aqueous sodium bicarbonate was added to the reaction mixture and the mixture was extracted with ethyl acetate. The organic layer was washed with saturated brine and dried over magnesium sulfate. After concentration under reduced pressure, the residue was separated and purified by column chromatography (eluent, ethyl acetate→ethyl acetate:methanol=9:1) to give the title compound (99 mg) as pale-yellow crystals.

¹H-NMR (CDCl₃) δ: 3.90 (2H, q, J=9.0 Hz), 4.16-4.25 (2H, m), 4.43-4.50 (2H, m), 4.91-5.02 (1H, m), 6.32 (1H, d, J=3.0 Hz), 7.08 (1H, d, J=3.0 Hz), 7.11 (1H, d, J=8.7 Hz), 7.27-7.33 (1H, m), 7.4,2-7.47 (1H, m), 7.52-7.58 (2H, m), 7.64-7.67 (1H, m), 7.83 (1H, d, J=2.7 Hz), 8.35 (1H, s), 9.45 (1H, s).

### Example C-121

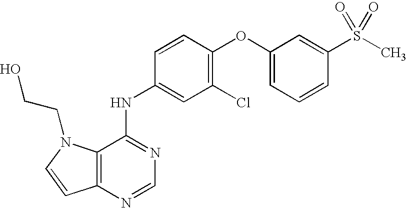

### Production of 2-[4-({3-chloro-4-[3-(methylsulfonyl)phenoxy]phenyl}amino)-5H-pyrrolo[3,2-d]pyrimidin-5-yl]ethanol

### (i) Production of 2-chloro-1-[3-(methylthio)phenoxy]-4-nitrobenzene

To a solution of 3-mercaptophenol (3.0 g) and triethylamine (3.64 mL) in N,N-dimethylformamide (30 mL) was added methyl iodide (1.48 mL) at 0° C. The reaction mixture was stirred at 0° C. for 1 hr and at room temperature for 30 min, 3-chloro-4-fluoronitrobenzene (4.18 g) and potassium carbonate (3.29 g) were added to the reaction mixture. The reaction mixture was stirred at room temperature for 14 hr, water was added to the reaction mixture, and the mixture was extracted with ethyl acetate. The organic layer was washed successively with water and saturated brine, and dried over magneium sulfate. After Concentration under reduced pressure, the residue was separated and purified by column chromatography (eluent, hexane:ethyl acetate=19:1→3:1) to give the title compound (2.84 g) as a yellow oil.

¹H-NMR (CDCl₃) δ: 2.49 (3H, s), 6.80-6.84 (1H, m), 6.91 (1H, d, J=9.3 Hz), 6.96-6.97 (1H, m) 7.11-7.15 (1H, m), 7.32-7.37 (1H, m), 8.07 (1H, dd, J=2.7 Hz, 9.3 Hz), 8.39 (1H, d, J=2.7 Hz).

### (ii) Production of 2-chloro-1-[3-(methylsulfonyl)phenoxy]-4-nitrobenzene

To a solution of 2-chloro-1-[3-(methylthio)phenoxy]-4-nitrobenzene (.2.84 g) in ethyl acetate (50 mL) was added 70% 3-chloroperbenzoic acid (5.21 g) at 0° C. The reaction mixture was stirred at 0° C. for 1 hr, aqueous sodium thiosulfate solution was added to the reaction mixture, and the mixture was stirred at room temperature for 1 hr. The mixture was extracted with ethyl acetate, the organic layer was washed successively with aqueous sodium bicarbonate and saturated brine, and dried over magnesium sulfate. After concentration under reduced pressure, the residue was separated and purified by column chromatography (eluent, hexane:ethyl acetate=4:1→2:3) to give the title compound (2.79 g) as colorless crystals.

¹H-NMR (CDCl₃) δ: 3.09 (3H, s), 7.02 (1H, d, J=8.7 Hz), 7.32-7.36 (1H, m), 7.61-7.67 (2H, m), 7.79-7.83 (1H, m), 8.13 (1H, dd, J=2.4 Hz, 8.7 Hz), 8.42 (1H, d, J=2.7 Hz).

### (iii) Production of 3-chloro-4-[3-(methylsulfonyl)phenoxy]aniline

Using 2-chloro-1-[3(methylsulfonyl)phenoxy]-4-nitrobenzene (2.90 g), reduced iron (2.50 g), calcium chloride (0.50 g) and 15% water-containing ethanol (90 mL) and in the same manner as in Example C-120(iii), the title compound (2.29 g) was obtained as pale-yellow crystals.

¹H-NMR (CDCl₃) δ: 3.03 (3H, s), 3.74 (2H, br s), 6.59 (1H, dd, J=2.7 Hz, 8.4 Hz), 6.78,(1H, d, J=2.7 Hz), 6.93 (1H, d, J=8.4 Hz), 7.13-7.17 (1H, m), 7.36-7.38 (1H, m), 7.44-7.50 (1H, m), 7.56-7.60 (1H, m).

### (iv) Production of 2-[4-({3-chloro-4-[3-(methylsulfonyl)phenoxy]phenyl}amino)-5H-pyrrolo[3,2-d]pyrimidin-5-yl]ethanol

A solution of 2-(4-chloro-5H-pyrrolo[3,2-d]pyrimidin-5-yl)ethyl benzoate (100 mg) and 3-chloro-4-[3-(methylsulfonyl)phenoxy]aniline (98.3 mg) in isopropyl alcohol (2.0 mL) was stirred at 80° C. for 3 days. The reaction mixture was cooled to room temperature, 1N aqueous sodium hydroxide solution (1.0 mL) was added thereto. The reaction mixture was stirred at room temperature for 2 hr, water was added thereto, and the mixture was extracted with ethyl acetate. The organic layer was washed with saturated brine and dried over magnesium sulfate. After concentration under reduced pressure, the residue was separated and purified by column chromatography (eluent, ethyl acetate→ethyl acetate:methanol=9:1) to give the title compound (116 mg) as colorless crystals.

¹H-NMR (CDCl₃) δ: 3.06 (3H, s), 4.18 (2H, t, J=4.7 Hz), 4.44 (2H, t, J=4.7 Hz), 6.27 (1H, d, J=3.0 Hz), 7.05 (1H, d, J=3.0 Hz), 7.11 (1H, d, J=9.0 Hz), 7.22-7.28 (1H, m), 7.43-7.45 (1H, m), 7.50-7.55 (2H, m), 7.61-7.64 (1H, m), 7.83 (1H, d, J=2.7 Hz), 8.31 (1H, s), 9.50 (1H, s).

### Example C-122

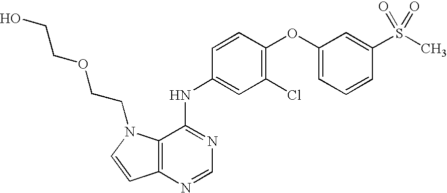

### Production of 2-{2-[4-({3-chloro-4-[3-(methylsulfonyl)phenoxy]phenyl}amino)-5H-pyrrolo[3,2-d]pyrimidin-5-yl]ethoxy}ethanol

Using 2-[2-(4-chloro-5H-pyrrolo[3,2-d]pyrimidin-5-yl)ethoxy]ethyl benzoate (100 mg), 3-chloro-4-[3-(methylsulfonyl)phenoxy]aniline (86.1 mg), isopropyl alcohol (2.0 mL) and 1N aqueous sodium hydroxide solution (1.0 mL) and in the same manner as in Example C-121(iv), the title compound (117 mg) was obtained as colorless crystals.

¹H-NMR (CDCl₃) δ: 1.78-1.85 (1H, m), 3.05 (3H, s), 3.78-3.84 (4H, m), 4.03 (2H, t, J=4.4 Hz), 4.59 (2H, t, J=4.4 Hz), 6.64 (1H, d, J=3.3 Hz), 7.09 (1H, d, J=9.0 Hz), 7.22-7.27 (2H, m), 7.39-7.41 (1H, m), 7.48-7.53 (1H, m), 7.61 (1H, s), 7.63 (1H, dd, J=2.7 Hz, 9.0 Hz), 7.94 (1H, d, J=2.7 Hz), 8.53 (1H, s), 8.85 (1H, s).

### Example C-123

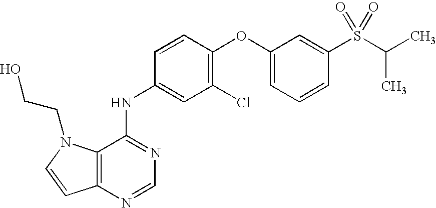

### Production of 2-[4-({3-chloro-4-[3-(isopropylsulfonyl)phenoxy]phenyl}amino)-5H-pyrrolo[3,2-d]pyrimidin-5-yl]ethanol

### (i) Production of 2-chloro-1-[3-(isopropylthio)phenoxy]-4-nitrobenzene

To a solution of 3-mercaptophenol (1.5 g) and sodium tert-butoxide (1.37 g) in N,N-dimethylformamide (25 mL) was added 2-bromopropane (1.23 mL) at room temperature. The reaction mixture was stirred at room temperature for 16 hr, sodium tert-butoxide (1.37 g), and 3-chloro-4-fluoronitrobenzene (1.88 g) were added to the reaction mixture, and the mixture was stirred for 4 hr. 3-Chloro-4-fluoronitrobenzene (0.17 g) was further added to the reaction mixture and the mixture was stirred at room temperature for 2 hr. Water was added to the reaction mixture, and the mixture was extracted with ethyl acetate. The organic layer was washed successively with water and saturated brine, and dried over magnesium sulfate. After concentration under reduced pressure, the residue was separated and purified by column chromatography (eluent, hexane:ethyl acetate=19:1→4:1) to give the title compound (3.36 g) as a yellow oil.

¹H-NMR (CDCl₃) δ: 1.32 (6H, d, J=6.9 Hz), 3.42 (1H, quintet, J=6.9 Hz), 6.88-6.92 (2H, m), 7.07-7.08 (1H, m), 7.21-7.27 (1H, m), 7.32-7.37 (1H, m), 8.06 (1H, dd, J=2.7 Hz, 9.3 Hz), 8.38 (1H, d, J=2.7 Hz).

### (ii) Production of 2-chloro-1-[3-(isopropylsulfonyl)phenoxy]-4-nitrobenzene

Using 2-chloro-1-[3-(isopropylthio)phenoxy]-4-nitrobenzene (3.36 g), 70% 3-chloroperbenzoic acid (5.63 g) and ethyl acetate (50 mL) and in the same manner as in Example C-121(ii), the title compound (3.36 g) was obtained as pale-yellow crystals.

¹H-NMR (CDCl₃) δ: 1.32 (6H, d, J=6.9 Hz), 3.22 (1H, quintet, J=6.9 Hz), 6.99 (1H, d, J=9.0 Hz), 7.33-7.37 (1H, m), 7.54-7.55 (1H, m), 7.61-7.66 (1H, m), 7.74-7.77 (1H, m), 8.13 (1H, dd, J=2.7 Hz, 9.0 Hz), 8.41 (1H, d, J=2.7 Hz).

### (iii) Production of 3-chloro-4-[3-(isopropylsulfonyl)phenoxy]aniline

Using 2-chloro-1-[3-(isopropylsulfonyl)phenoxy]-4-nitrobenzene (3.30 g), reduced iron (2.59 g), calcium chloride (0.52 g) and 15% water-containing ethanol (100 mL) and in the same manner as in Example C-120(iii), the title compound (3.00 g) was obtained as a yellow oil.

¹H-NMR (CDCl₃) 67 : 1.28 (6H, d, J=6.9 Hz), 3.16 (1H, quintet, J=6.9 Hz), 3.73 (2H, br s), 6.59 (1H, dd, J=2.7 Hz, 8.7 Hz), 6.78 (1H, d, J ₇ 2.7 Hz), 6.93 (1H, d, J=8.7 Hz), 7.14-7.19 (1H, m), 7.28-7.30 (1H, m), 7.44-7.54 (2H, m).

### (iv) Production of 2-[4-({3-chloro-4-[3-(isopropylsulfonyl)phenoxy]phenyl}amino)-5H-pyrrolo[3,2-d]pyrimidin-5-yl]ethanol

Using 2-(4-chloro-5H-pyrrolo[3,2d]pyrimidin-5-yl)ethyl benzoate (100 mg), 3-chloro-4-[3-(isopropylsulfonyl)phenoxy]aniline (140 mg), isopropyl alcohol (2.0 mL) and 1N aqueous sodium hydroxide solution (1.0 mL) and in the same manner as in Example C-121(iv), the title compound (127 mg) was obtained as pale-yellow crystals.

¹H-NMR (CDCl₃) δ: 1.30 (6H, d, J=6.9 Hz), 3.19 (1H, quintet, J=6.9 Hz), 4.16 (2H, t, J=4.5 Hz), 4.43 (2H, t, J=4.5 Hz), 5.30-5.71 (1H, m), 6.26 (1H, d, J=3.0 Hz), 7.05 (1H, d, J=3.0 Hz), 7.10 (1H, d, J=8.7 Hz), 7.23-7.30 (1H, m), 7.35-7.37 (1H, m), 7.48-7.61 (3H, m), 7.84 (1H, d, J=2.4 Hz), 8.31 (1H, s), 9.53 (1H, s).

### Example C-124

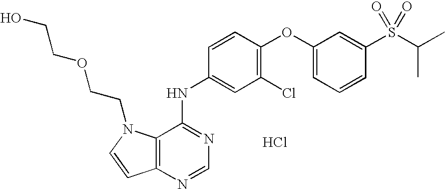

### Production of 2-{2-[4-({3-chloro-4-[3-(isopropylsulfonyl)phenoxy]phenyl}amino)-5H-pyrrolo[3,2-d]pyrimidin-5-yl]ethoxy}ethanol hydrochloride

Using 2-[2-(4-chloro-5H-pyrrolo[3,2-d]pyrimidin-5-yl)ethoxy]ethyl benzoate (100 mg), 3-chloro-4-[3-(isopropylsulfonyl)phenoxy]aniline (113 mg), isopropyl alcohol (2.0 mL) and 1N aqueous sodium hydroxide solution (1.0 mL) and in the same manner as in Example C-121(iv), a pale-yellow oil was obtained. To a solution of the obtained oil in ethanol (5.0 mL) was added 4N hydrogen chloride/ethyl acetate solution (0.50 mL) at room temperature. The mixture was concentrated under reduced pressure, and resulting crystals were collected by filtration. The crystals were washed with ethyl acetate to give the title compound (130 mg) as pale-yellow crystals.

¹H-NMR (DMSO-d₆) δ: 1.15 (6H, d, J=6.9 Hz), 3.38-3.54 (5H, m), 3.82-3.89 (2H, m), 4.75-4.83 (2H, m), 6.69 (1H, d, J=3.0 Hz), 7.23-7.25 (1H, m), 7.39-7.44 (2H, m), 7.61-7.75 (3H, m), 7.99-8.03 (2H, m), 8.73 (1H, s), 9.84 (1H, br s).

### Example C-125

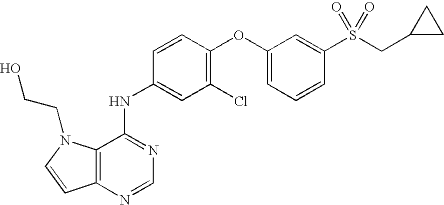

### Production of 2-{4-[(3-chloro-4-{3-[(cyclopropylmethyl)sulfonyl]phenoxy}phenyl)amino]-5H-pyrrolo[3,2-d]pyrimidin-5-yl}ethanol

### (i) Production of 2-chloro-1-{3-[(cyclopropylmethyl)thio]phenoxy}-4-nitrobenzene

To a solution of 3-mercaptophenol (1.5 g) and sodium tert-butoxide (1.26 g) in N,N-dimethylformamide (15 mL) was added 1-(bromomethyl)cyclopropane (1.27 mL) at room temperature. The reaction mixture was stirred at room temperature for 16 hr, sodium tert-butoxide (1.27 g) and 3-chloro-4-fluoronitrobenzene (1.88 g) were added to the reaction mixture, and the mixture was stirred for 3 days. Water was added to the reaction mixture, and the mixture was extracted with ethyl acetate. The organic layer was washed successively with water and saturated brine, and dried over magnesium sulfate. After concentration under reduced pressure, the residue was separated and purified by column chromatography (eluent, hexane:ethyl acetate=19:1→4:1) to give the title compound (2.93 g) as a yellow oil.

¹H-NMR (CDCl₃) δ: 0.25-0.30 (2H, m), 0.58-0.64 (2H, m), 0.99-1.13 (1H, m), 2.89 (2H, d, J=7.2 Hz), 6.84-6.89 (1H, m), 6.90 (1H, d, J=9.0 Hz), 7.05 (1H, t, J=2.1 Hz), 7.20-7.24 (2H, m), 7.31-7.36 (1H, m), 8.06 (1H, dd, J=2.7 Hz, 9.0 Hz), 8.39 (1H, d, J=2.7 Hz).

### (ii) Production of 2-chloro-1-{3-[(cyclopropylmethyl)sulfonyl]phenoxy}-4-nitrobenzene

Using 2-chloro-1-{3-[(cyclopropylmethyl)thio]phenoxy}-4-nitrobenzene (2.93 g), 70% 3-chloroperbenzoic acid (4.73 g) and ethyl acetate (60 mL) and in the same manner as in Example C-121(ii), the title compound (2.90 g) was obtained as colorless crystals.

¹H-NMR (CDCl₃) δ: 0.14-0.20 (2H, m), 0.57-0.63 (2H, m), 0.95-1.09 (1H, m), 3.05 (2H, d, J=7.2 Hz), 7.00 (1H, d, J=9.0 Hz), 7.33-7.36 (1H, m), 7.59-7.66 (2H, m), 7.78-7.82 (1H, m), 8.13 (1H, dd, J=2.7 Hz, 9.0 Hz), 8.41 (1H, d, J=2.7 Hz).

### (iii) Production of 3-chloro-4-{3-[(cyclopropylmethyl)sulfonyl]phenoxy}aniline

Using 2-chloro-1-{3-[(cyclopropylmethyl)sulfonyl]phenoxy}-4-nitrobenzene (2.84 g), reduced iron (2.57 g), calcium chloride (0.51 g) and 15% watercontaining ethanol (85 mL) and in the same manner as in. Example C-120(iii), the title compound (2.60 g) was obtained a yellow oil.

¹H-NMR (CDCl₃) δ: 0.10-0.16 (2H, m), 0.52-0.58 (2H, m), 0.90-1.06 (1H, m), 2.99 (2H, d, J=7.5 Hz), 3.74 (2H, br s), 6.59 (1H, dd, J=2.7 Hz, 8.7 Hz), 6.78 (1H, d, J=2.7 Hz), 6.93 (1H, d, J=8.7 Hz), 7.15-7.19 (1H, m), 7.34-7.35 (1H, m), 7.44-7.49 (1H, m), 7.55-7.59 (1H, m).

### (iv) Production of 2-{4-[(3-chloro-4-{3-[(cyclopropylmethyl)sulfonyl]phenoxy}phenyl)amino]-5H-pyrrolo[3,2-d]pyrimidin-5-yl}ethanol

Using 2-(4-chloro-5H-pyrrolo[3,2-d]pyrimidin-5-yl)ethyl benzoate (100 mg), 3-chloro-4-{3-[(cyclopropylmethyl)sulfonyl]phenoxy}aniline (123 mg), isopropyl alcohol (2.0 mL), 1-methyl-2-pyrrolidone (2.0 mL) and 1N aqueous sodium hydroxide solution (1.0 mL) and in the same manner as in Example C-121(iv), the title compound (126 mg) was obtained as colorless crystals.

¹H-NMR (CDCl₃) δ: 0.11-0.19 (2H, m), 0.53-0.63 (2H, m), 0.94-1.05 (1H, m), 3.01 (2H, d, J=7.2 Hz), 4.13-4.21 (2H, m), 4.39-4.47 (2H, m), 5.11-5.31 (1H, m), 6.29 (1H, d, J=3.3 Hz), 7.06 (1H, d, J=3.3 Hz), 7.10 (1H, d, J=8.7 Hz), 7.24-7.30 (1H, m), 7.40-7.42 (1H, m), 7.48-7.54 (2H, m), 7.60-7.63 (1H, m), 7.83 (1H, d, J=2.4 Hz), 8.32 (1H, s), 9.48 (1H, s).

### Example C-126

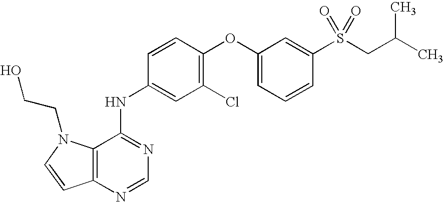

### Production of 2-[4-({3-chloro-4-[3-(isobutylsulfonyl)phenoxy]phenyl}amino)-5H-pyrrolo[3,2-d]pyrimidin-5-yl]ethanol

### (i) Production of 2-chloro-1-[3-(isobutylthio)phenoxy]-4-nitrobenzene

To a solution of 3-mercaptophenol (1.5 g) and sodium tert-butoxide (1.26 g) in N,N-dimethylformamide (15 mL) was added 1-bromo-2-methylpropane (1.42 mL) at room temperature. The reaction mixture was stirred at room temperature for 2 days, sodium tert-butoxide (1.26 g) and 3-chloro-4-fluoronitrobenzene (2.08 g) were added to the reaction mixture and the mixture was stirred for 6 hr. Water was added to the reaction mixture, and the mixture was extracted with ethyl acetate. The organic layer was washed successively with water and saturated brine, and dried over magnesium sulfate. After concentration under reduced pressure, the residue was separated and purified by column chromatography (eluent, hexane:ethyl acetate=19:1→3:1) to give the title compound (3.63 g) as a yellow oil.

¹H-NMR (CDCl₃) δ: 1.04 (6H, d, J=6.6 Hz), 1.83-1.96 (1H, m), 2.82 (2H, d, J=6.3 Hz), 6.81-6.84 (1H, m), 6.89 (1H, d, J=9.0 Hz), 6.99-7.01 (1H, m), 7.15-7.19 (1H, m), 7.29-7.34 (1H, m), 8.05 (1H, dd, J=2.4 Hz, 9.0 Hz), 8.37 (1H, d, J=2.4 Hz).

### (ii) Production of 2-chloro-1-[3-(isobutylsulfonyl)phenoxy]-4-nitrobenzene

Using 2-chloro-1-[3-(isobutylthio)phenoxy]-4-nitrobenzene (3.63 g), 70% 3-chloroperbenzoic acid (5.80 g) and ethyl acetate (50 mL) and in the same manner as in Example C-121(ii), the title compound (3.88 g) was obtained as colorless crystals.

¹H-NMR (CDCl₃) δ: 1.08 (6H, d, J=6.6 Hz), 2.20-2.23 (1H, m), 3.00 (2H, d, J=6.6 Hz), 7.00 (1H, d, J=9.0 Hz), 7.31-7.35 (1H, m), 7.58 (1H, t, J=2.1 Hz), 7.60-7.66 (1H, m), 7.76-7.80 (1H, m), 8.13 (1H, dd, J=2.7 Hz, 9.0 Hz), 8.41 (1H, d, J=2.7 Hz).

### (iii) Production of 3-chloro-4-[3-(isobutylsulfonyl)phenoxy]aniline

Using 2-chloro-1-[3-(isobutylzulfonyl)phenoxy]-4-nitrobenzene (3.88 g), reduced iron (2.93 g), calcium chloride (0.58 g) and 15% water-containing ethanol (120 mL) and in the same manner as in Example C-120(iii), the title compound (3.40 g) was obtained as a yellow oil.

¹H-NMR (CDCl₃) δ: 1.04 (6H, d, J=6.6 Hz), 2.13-2.28 (1H, m), 2.96 (2H, d, J=6.6 Hz), 3.74 (2H, br s), 6.60 (1H, dd, J=2.7 Hz, 8.7 Hz), 6.79 (1H, d, J=2.7 Hz), 6.93 (1H, d, J=8.7 Hz), 7.13-7.18 (1H, m), 7.34 (1H, t, J=2.1 Hz), 7.47 (1H, t, J=8.0 Hz), 7.54-7.57 (1H, m).

### (iv) Production of 2-[4-({3-chloro-4-[3-(isobutylsulfonyl)phenoxy]phenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (100 mg), 3-chloro-4-[3-(isobutylsulfonyl)phenoxy]aniline (123 mg), isopropyl alcohol (5.0 mL) and 1N aqueous sodium hydroxide solution (2.0 mL) and in the same manner as in Example C-121(iv), the title compound (112 mg) was obtained as colorless crystals.

¹H-NMR (CDCl₃) δ: 1.06 (6H, d, J=6.9 Hz), 2.18-2.29 (1H, m), 2.98 (2H,d, J=6.6 Hz), 4.12-4.21 (2H, m), 4.39-4.48 (2H, m), 5.45-5.60 (1H, m), 6.25 (1H, d, J=3.0 Hz), 7.04 (1H, d, J=3.0 Hz), 7.09 (1H, d, J=8.7 Hz), 7.23-7.27 (1H, m), 7.39-7.41 (1H, m), 7.48-7.53 (2H, m), 7.57-7.61 (1H, m), 7.83 (1H, d, J=2.4 Hz), 8.30 (1H, s), 9.50 (1H, s).

### Example C-127

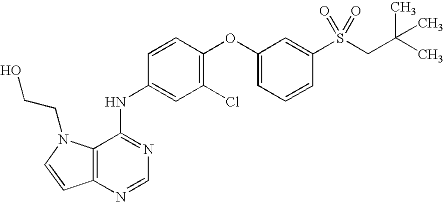

### Production of 2-{4-[(3-chloro-4-{3-[(2,2-dimethylpropyl)sulfonyl]phenoxy}phenyl)amino]-5H-pyrrolo[3,2-d]pyrimidin-5-yl}ethanol

### (i) Production of 2-chloro-1-{3-[(2,2-dimethylpropyl)thio]phenoxy}-4-nitrobenzene

To a solution of 3-mercaptophenol (1.5 g) and sodium tert-butoxide (1.26 g) in N,N-dimethylformamide (15 mL) was added 1-bromo-2,2-dimethylpropane (1.64 mL) at room temperature. After stirring at 70° C. for 24 hr, the mixture was cooled to room temperature, sodium tert-butoxide (1.26 g) and 3-chloro-4-fluoronitrobenzene (2.08 g) were added thereto, and the mixture was stirred at room temperature for 3 days. Water was added to the reaction mixture, and the mixture was extracted with ethyl acetate. The organic layer was washed successively with water and saturated brine, and dried over magnesium sulfate. After concentration under reduced pressure, the residue was separated and purified by basic silica gel column chromatography (eluent, hexane:ethyl acetate=19:1→47:3) to give the title compound (2.80 g) as a yellow oil.

¹H-NMR (CDCl₃) δ: 1.05 (9H, s), 2.89 (2H, s), 6.80-6.84 (1H, m), 6.88 (1H, d, J=9.0 Hz), 7.02-7.04 (1H, m), 7.18-7.22 (1H, m), 7.28-7.33 (1H, m), 8.05 (1H, dd, J=3.0 Hz, 9.0 Hz), 8.37 (1H, d, J=3.0 Hz).

### (ii) Production of 2-chloro-1-{3-[(2,2-dimethylpropyl)sulfonyl]phenoxy}-4-nitrobenzene

Using 2-chloro-1-{3-[(2,2-dimethylpropyl)thio]phenoxy}-4-nitrobenzene (2.80 g), 70% 3-chloroperbenzoic acid (4.32 g) and ethyl acetate (56 mL) and in the same manner as in Example C-121(ii), the title compound (3.04 g) was obtained as pale-yellow crystals.

¹H-NMR (CDCl₃) δ: 1.20 (9H, s), 3.05 (2H, s), 7.00 (1H, d, J=9.0 Hz), 7.30-7.34 (1H, m), 7.59-7.66 (2H, m), 7.77-7.81 (1H, m), 8.13 (1H, dd, J=2.7 Hz, 9.0 Hz), 8.42 (1H, d, J=2.7 Hz).

### (iii) Production of 3-chloro-4-{3-[(2,2-dimethylpropyl)sulfonyl]phenoxy}aniline

Using 2-chloro-1-{3-[(2,2-dimethylpropyl)sulfonyl]phenoxy}-4-nitrobenzene (3.0 g), reduced iron (2.18 g), calcium chloride (0.43 g) and 15% water-containing ethanol (90 mL) and in the same manner as in Example C-120(iii), the title compound (2.34 g) was obtained as a pale-yellow amorphous.

¹H-NMR (CDCl₃) δ: 1.17 (9H, s), 3.01 (2H, s), 3.73 (2H, br s), 6.59 (1H, dd, J=2.7 Hz, 9.0 Hz), 6.78 (1H, d, J=2.7 Hz), 6.92 (1H, d, J=9.0 Hz), 7.08-7.14 (1H, m), 7.35-7.36 (1H, m), 7.42-7.47 (1H, m), 7.53-7.57 (1H, m).

### (iv) Production of 2-{4-[(3-chloro-4-{3-[(2,2-dimethylpropyl)sulfonyl]phenoxy}phenyl)amino]-5H-pyrrolo[3,2-d]pyrimidin-5-yl}ethanol

Using 2-(4-chloro-5H-pyrrolo[3,2-d]pyrimidin-5-yl)ethyl benzoate (100 mg), 3-chloro-4-{3-[(2,2-dimethylpropyl)sulfonyl]phenoxy}aniline (140 mg), isopropyl alcohol (3.0 mL) and 1N aqueous sodium hydroxide solution (1.5 mL) and in the same manner as in Example C-121(iv), the title compound (131 mg) was obtained as colorless crystals.

¹H-NMR (CDCl₃) δ: 1.19 (9H, s), 3.04 (2H, s), 4.11-4.20 (2H, m), 4.35-5.46 (2H, m), 5.81-5.96 (1H, m), 6.20 (1H, d, J=3.3 Hz), 7.02 (1H, d, J=3.3 Hz), 7.08 (1H, d, J=8.7 Hz), 7.20-7.27 (1H, m), 7.41-7.43 (1H, m), 7.46-7.52 (2H, m), 7.56-7.63 (1H, m), 7.83 (1H, d, J=2.7 Hz), 8.27 (1H, s), 9.55 (1H, s).

### Example C-128

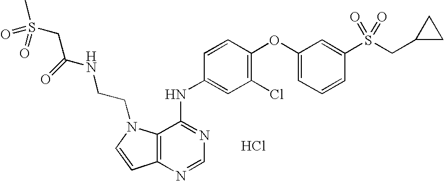

### Production of N-(2-{4-[(3-chloro-4-{3-[(cyclopropylmethyl)sulfonyl]phenoxy}phenyl)amino]-5H-pyrrolo[3,2-d]pyrimidin-5-yl}ethyl)-2-(methylsulfonyl)acetamide hydrochloride

### (i) Production of tert-butyl(2-{4-[(3-chloro-4-{3-[(cyclopropylmethyl)sulfonyl]phenoxy}phenyl)amino]-5H-pyrrolo[3,2-d]pyrimidin-5-yl}ethyl)carbamate

A solution of tert-butyl[2-(4-chloro-5H-pyrrolo[3,2-d]pyrimidin-5-yl)ethyl]carbamate (1.00 g) and 3-chloro-4-{3-[(cyclopropylmethyl)sulfonyl]phenoxy}aniline (1.14 g) in isopropyl alcohol (10 mL) was stirred at 80° C. for 14 hr. Aqueous sodium bicarbonate was added to the reaction mixture and the mixture was extracted with ethyl acetate. The organic layer was washed with saturated brine and dried over magnesium sulfate. After concentration under reduced pressure, the residue was separated and purified by column chromatography (eluent, hexane:ethyl acetate=1:1→ethyl acetate→ethyl acetate:methanol=9:1) to give the title compound (1.86 g) as pale-yellow crystals.

¹H-NMR (CDCl₃) δ: 0.10-0.20 (2H, m), 0.54-0.61 (2H, m), 0.92-1.06 (1H, m), 1.50 (9H, s), 3.01 (2H, d, J=6.9 Hz), 3.43-3.53 (2H, m), 4.43-4.52 (2H, m), 5.02-5.10 (1H, m), 6.61 (1H, d, J=3.0 Hz), 7.09 (1H, d, J=9.0 Hz), 7.19 (1H, d, J=3.0 Hz), 7.21-7.28 (1H, m), 7.46-7.52 (2H, m), 7.60-7.63 (1H, m), 7.91 (1H, dd, J=2.7 Hz, 9.0 Hz), 8.06 (1H, d, J=2.7 Hz), 8.51 (1H, s), 8.62 (1H, br s).

### (ii) Production of 5-(2-aminoethyl)-N-(3-chloro-4-{3-[(cyclopropylmethyl)sulfonyl]phenoxy}phenyl)-5H-pyrrolo[3,2-d]pyrimidin-4-amine dihydrochloride

A mixture of tert-butyl(2-{4-[(3-chloro-4-{3-[(cyclopropylmethyl)sulfonyl]phenoxy}phenyl)amino]-5H-pyrrolo[3,2-d]pyrimidin-5-yl}ethyl)carbamate (1.86 g), 6N hydrochloric acid (5.0 mL) and ethanol (20 mL) was stirred at 60° C. for 3 days. After concentration under reduced pressure, ethanol was added to the residue. The mixture was concentrated under reduced pressure, and the resulting crystals were collected by filtration and washed with diisopropyl ether to give the title compound (1.66 g) as pale-yellow crystals.

¹H-NMR (DMSO-d₆) δ: 0.09-0.14 (2H, m), 0.41-0.48 (2H, m), 0.76-0.88 (1H, m), 3.22-3.38 (4H, m), 4.94-5.05 (2H, m), 6.74 (1H, d, J=2.7 Hz), 7.30-7.45 (3H, m), 7.61-7.74 (3H, m), 7.92-7.97 (1H, m), 8.00-8.08 (1H, m), 8.23-8.32 (3H, m), 8.72 (1H, s), 9.99 (1H, br s).

### (iii) Production of N-(2-{4-[(3-chloro-4-{3-[(cyclopropylmethyl)sulfonyl]phenoxy}phenyl)amino]-5H-pyrrolo[3,2-d]pyrimidin-5-yl}ethyl)-2-(methylsulfonyl)acetamide hydrochloride

A mixture of 5-(2-aminoethyl)-N-(3-chloro-4-{3-[(cyclopropylmethyl)sulfonyl]phenoxy}phenyl)-5H-pyrrolo[3,2-d]pyrimidin-4-amine dihydrochloride (100 mg), methylsulfonylacetic acid (48.4 mg), 1-ethyl-3-(3-dimethylaminopropyl)carbodiimide hydrochloride (101 mg), 1-hydroxybenzotriazole monohydrate (80 mg) and triethylamine (0.073 mL) in N,N-dimethylformamide (5.0 mL) was stirred at room temperature for 20 hr. Water was added to the reaction mixture and the mixture was extracted with ethyl acetate. The organic layer was washed successively with water and saturated brine, and dried over magnesium sulfate. After concentration under reduced pressure, the residue was separated and purified by basic silica gel column chromatography (eluent, ethyl acetate→ethyl acetate:methanol=85:15) to give a pale-yellow amorphous. To a solution of the obtained amorphous in ethnol (5.0 mL) was added 4N hydrogen chloride/ethyl acetate solution (0.5 mL) at room temperature. After stirring at room temperature for 1 hr, the resulting crystals were collected by filtration and washed with ethyl acetate to give the title compound (81 mg) as colorless crystals.

¹H-NMR (DMSO-d₆) δ: 0.06-0.13 (2H, m), 0.40-0.47 (2H, m), 0.75-0.90 (1H, m), 3.06 (3H, s), 3.30 (2H, d, J=6.9 Hz), 3.49-3.60 (2H, m), 4.06 (2H, s), 4.64-4.73 (2H, m), 6.66 (1H, d, J=2.7 Hz), 7.30-7.44 (3H, m), 7.65-7.74 (3H, m), 7.91-7.99 (2H, m), 8.68-8.79 (2H, m), 8.86 (1H, br s).

### Example C-129

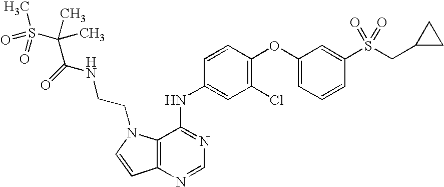

### Production of N-(2-{4-[(3-chloro-4-{3-[(cyclopropylmethyl)sulfonyl]phenoxy}phenyl)amino]-5H-pyrrolo[3,2-d]pyrimidin-5-yl}ethyl)-2-methyl-2-(methylsulfonyl)propanamide

A mixture of 5-(2-aminoethyl)-N-(3-chloro-4-{3-[(cyclopropylmethyl)sulfonyl]phenoxy}phenyl)-5H-pyrrolo[3,2-d]pyrimidin-4-amine dihydrochloride (120 mg), 2-methyl-2-(methylsulfonyl)propanoic acid (52 mg), 1-ethyl-3-(3-dimethylaminopropyl)carbodiimide hydrochloride (90 mg), 1-hydroxybenzotriazole monohydrate (72 mg) and triethylamine (0.088 mL) in N,N-dimethylformamide (5.0 mL) was stirred at room temperature for 2 days. Water was added to the reaction mixture and the mixture was extracted with ethyl acetate. The organic layer was washed successively with mater and saturated brine, and dried over magnesium sulfate. After concentration under reduced pressure, the residue was separated and purified by basic silica gel column chromatography (eluent, ethyl acetate→ethyl acetate:methanol=85:15) to give the title compound (120 mg) as pale-yellow crystals.

¹H-NMR (CDCl₃) δ: 0.11-0.18 (2H, m), 0.54-0.61 (2H, m), 0.92-1.07 (1H, m), 1.70 (6H, s), 2.93 (3H, s), 3.01 (2H, d, J=7.2 Hz), 3.64-3.74 (2H, m), 4.43-4.52 (2H, m), 6.64 (1H, d, J=3.3 Hz), 6.99 (1H, d, J=9.0 Hz), 7.21 (1H, d, J.=3.3 Hz), 7.22-7.32 (2H, m), 7.42-7.44 (1H, m), 7.51 (1H, t, J=8.1 Hz), 7.60-7.64 (1H, m), 7.89 (1H, dd, J=2.7 Hz, 9.0 Hz), 8.07 (1H, d, J=2.7 Hz), 8.37 (1H, s), 8.53 (1H, s).

### Example C-130

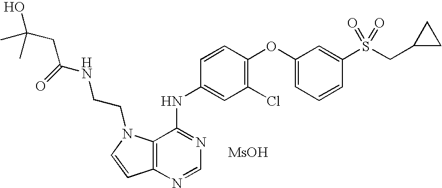

### Production of N-(2-{4-[(3-chloro-4-{3-[(cyclopropylmethyl)sulfonyl]phenoxy}phenyl)amino]-5H-pyrrolo[3,2-d]pyrimidin-5-yl}ethyl)-3-hydroxy-3-methylbutanamide methanesulfonate

A mixture of 5-(2-aminoethyl)-N-(3-chloro-4-{3-[(cyclopropylmethyl)sulfonyl]phenoxy}phenyl)-5H-pyrrolo[3,2-d]pyrimidin-4-amine dihydrochloride (120 mg), 3-hydroxy-3-methylbutanoic acid (49.7 mg), 1-ethyl-3-(3-dimethylaminopropyl)carbodiimide hydrochloride (121 mg), 1-hydroxybenzotriazole monohydrate (97 mg) and triethylamine (0.090 mL) in N,N-dimethylformamide (5.0 mL) was stirred at room temperature for 20 hr. Water was added to the reaction mixture and the mixture was extracted with ethyl acetate. The organic layer was washed successively with water and saturated brine, and dried over magnesium sulfate. After concentration under reduced pressure, the residue was separated and purified by basic silica gel column chromatography (eluent, ethyl acetate→ethyl acetate:methanol=4:1) to give a colorless amorphous form. To a solution of the obtained amorphous form in ethyl acetate (5.0 mL) was added methanesulfonic acid (12.4 μL) at room temperature. After stirring at room temperature for 1 hr, the resulting crystals were collected by filtration and washed with ethyl acetate to give the title compound (116 mg) as colorless crystals.

¹H-NMR (DMSO-d₆) δ: 0.08-0.14 (2H, m), 0.41-0.47 (2H, m), 0.75-0.88 (1H, m), 1.12 (6H, s), 2.20 (2H, s), 2.30 (3H, s), 3.29 (2H, d, J=7.2 Hz), 3.43-3.56 (2H, m), 4.62 (2H, t, J=7.5 Hz), 6.66 (1H, d, J=3.0 Hz), 7.31-7.45 (3H, m), 7.64-7.76 (3H, m), 7.93-8.01 (2H, m), 8.34 (1H, t, J=5.4 Hz), 8.72 (1H, s), 10.14 (1H, br s).

### Example C-131

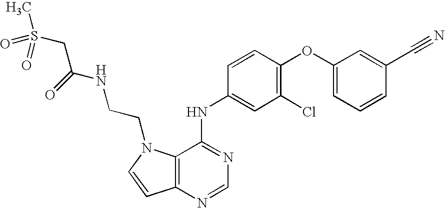

### Production of N-[2-(4-{[3-chloro-4-(3-cyanophenoxy)phenyl]amino}-5H-pyrrolo[3,2-d]pyrimidin-5-yl)ethyl]-2-(methylsulfonyl)acetamide

Using 3-(4-amino-2-chlorophenoxy)benzonitrile (115 mg), tert-butyl[2-(4-chloro-5H-pyrrolo[3,2-d]pyrimidin-5-yl)ethyl]carbamate (125 mg), isopropyl alcohol (2.0 mL), methanol (2 mL), 4N hydrogen chloride/ethyl acetate solution (3.0 mL), methylsulfonylacetic acid (170 mg), 1-ethyl-3-(3-dimethylaminopropyl)carbodiimide hydrochloride (360 mg), 1-hydroxybenzotriazole (15 mg), triethylamine (0.86 mL) and N,N-dimethylformamide (15 mL) and in the same manner as in Example C-53(ii), the title compound (169 mg) was obtained as colorless crystals.

¹H-NMR (DMSO-d₆) δ: 3.10 (3H, s), 3.44-3.49 (2H, m), 4.05 (2H, s), 4.55-4.60 (2H, m), 6.51-6.52 (1H, m), 7.24-8.01 (8H, m), 8.38 (1H, s), 8.66-8.69 (1H, m), 8.81 (1H, s).

### Example C-132

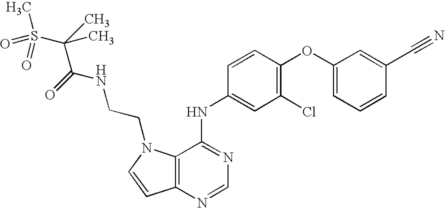

### Production of N-[2-(4-{[3-chloro-4-(3-cyanophenoxy)phenyl]amino}-5H-pyrrolo[3,2-d]pyrimidin-5-yl)ethyl]-2-methyl-2-(methylsulfonyl)propanamide

Using 3-(4-amino-2-chlorophenoxy)benzonitrile (115 mg), tert-butyl[2-(4-chloro-5H-pyrrolo[3,2-d]pyrimidin-5-yl)ethyl]carbamate (125 mg), isopropyl alcohol (2.0 mL), methanol (2 mL), 4N hydrogen chloride/ethyl acetate solution (3.0 mL), 2-methyl-2-(methylsulfonyl)propanoic acid (210 mg), 1-ethyl-3-(3-dimethylaminopropyl)carbodiimide hydrochloride (370 mg), 1-hydroxybenzotriazole (40 mg), triethylamine (1.0 mL) and N,N-dimethylformamide (20 mL) and in the same manner as in Example C-53(ii), the title compound (173 mg) was obtained as pale-yellow crystals.

¹H-NMR (DMSO-d₆) δ: 1.41 (6H, s), 2.95 (3H, s), 3.42-3.49 (2H, m), 4.58-4.62 (2H, m), 6.50-6.51 (1H, m), 7.24-8.00 (8H, m), 8.20 (1H, br s), 8.40 (1H, s), 8.88 (1H, s).

### Example C-133

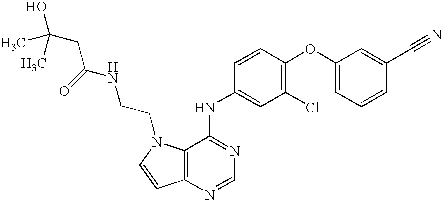

### Production of N-[2-(4-{[3-chloro-4-(3-cyanophenoxy)phenyl]amino}-5H-pyrrolo[3,2-d]pyrimidin-5-yl)ethyl]-3-hydroxy-3-methylbutanamide

Using 3-(4-amino-2-chlorophenoxy)benzonitrile (115 mg), tert-butyl[2-(4-chloro-5H-pyrrolo[3,2-d]pyrimidin-5-yl)ethyl]carbamate (125 mg), isopropyl alcohol (2.0 mL), methanol (2 mL), 4N hydrogen chloride/ethyl acetate solution (3.0 mL), 3-hydroxy-3-methylbutanoic acid (190 mg), 1-ethyl-3-(3-dimethylaminopropyl)carbodiimide hydrochloride (320 mg), 1-hydroxybenzotriazole (15 mg), triethylamine (0.5 mL) and N,N-dimethylformamide (20 mL) and in the same manner as in Example C-53(ii), the title compound (115 mg) was obtained as pale-yellow crystals.

¹H-NMR (DMSO-d₆) δ: 1.13 (6H, s), 2.20 (2H, s), 3.42-3.49 (2H, m), 4.50-4.55 (2H, m), 4.66 (1H, s), 6.50-6.51 (1H, m), 7.24-8.06 (8H, m), 8.24 (1H, br s), 8.35 (1H, s), 8.92 (1H, s).

### Example D-1

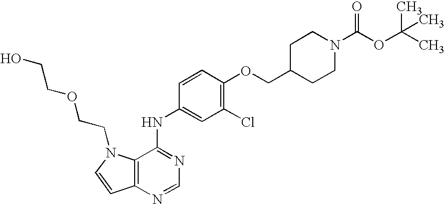

### Production of tert-butyl 4-{[2-chloro-4-({5-[2-(2-hydroxyethoxy)ethyl]-5H-pyrrolo[3,2-d]pyrimidin-4-yl}amino)phenoxy]methyl}piperidine-1-carboxylate

A mixture of 2-[2-(4-chloro-5H-pyrrolo[3,2-d]pyrimidin-5-yl)ethoxy]ethyl benzoate (156 mg), tert-butyl 4-[(4-amino-2-chlorophenoxy)methyl]piperidine-1-carboxylate (200 mg) and isopropyl alcohol (15 mL) was stirred at 80° C. overnight. After concentration, under reduced pressure, water and saturated aqueous sodium hydrogencarbonate were added to the reaction mixture and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:methanol=100:0→80:20). The objective fractions were concentrated under reduced pressure. The crude product was dissolved in methanol (5.0 mL), tetrahydrofuran (4.0 mL) and 1N aqueous sodium hydroxide solution (3.0 mL) were added and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate/hexane to give the title compound (74 mg) as crystals.

¹H-NMR (DMSO-d₆) δ: 1.13-1.27 (2H, m), 1.40 (9H, s), 1.76-1.80 (2H, m), 1.86-2.03 (1H, m), 2.65-2.86 (2H, m), 3.47 (4H, s), 3.78-4.01 (6H, m), 4.60-4.68 (3H, m), 6.47 (1H, d, J=3.1 Hz), 7.12 (1H, d, J=9.1 Hz), 7.50 (1H, dd, J=9.1, 2.7 Hz), 7.63 (1H, d, J=3.1 Hz), 7.78 (1H, d, J=2.7 Hz), 8.26 (1H, s), 8.68 (1H, br s).

### Example D-2

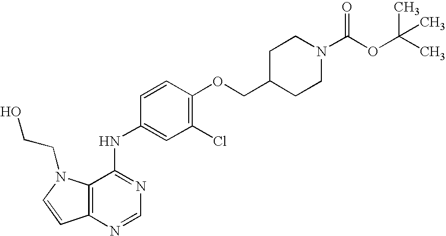

### Production of tert-butyl 4-[(2-chloro-4-{[5-(2-hydroxyethyl)-5H-pyrrolo[3,2-d]pyrimidin-4-yl]amino}phenoxy)methyl]piperidine-1-carboxylate

A mixture of 2-(4-chloro-5H-pyrrolo[3,2-d]pyrimidin-5-yl)ethyl benzoate (200 mg), tert-butyl 4-[(4-amino-2-chlorophenoxy)methyl]piperidine-1-carboxylate (293 mg) and isopropyl alcohol (5.0 mL) was stirred at 80° C. overnight. After concentration under reduced pressure, water and saturated aqueous sodium hydrogencarbonate were added to the reaction mixture and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:methanol=100:0→80:20). The objective fractions were concentrated under reduced pressure. The crude product was dissolved in methanol (5.0 mL), tetrahydrofuran (4.0 mL) and 1N aqueous sodium hydroxide solution (4.0 mL) were added to the mixture and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate/hexane to give the title compound (147 mg) as a white powder.

¹H-NMR (DMSO-d₆) δ: 1.13-1.27 (2H, m), 1.40 (9H, s), 1.76-1.80 (2H, m), 1.86-2.03 (1H, m), 2.65-2.86 (2H, m), 3.83-4.01 (6H, m), 4.50 (2H, t, J=4.1 Hz), 6.18-6.21 (1H, m), 6.46 (1H, d, J=3.1 Hz), 7.13 (1H, d, J=9.1 Hz), 7.46 (1H, dd, J=9.1, 2.7 Hz), 7.60 (1H, d, J=3.1 Hz), 7.75 (1H, d, J=2.7 Hz), 8.26 (1H, s), 9.53 (1H, br s).

### Example E-1

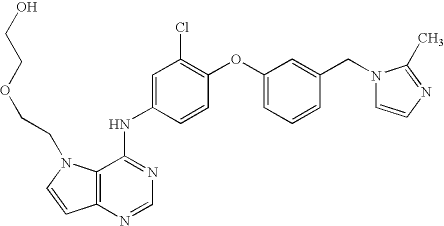

### Production of 2-(2-{4-[(3-chloro-4-{3-[(2-methyl-1H-imidazol-1-yl)methyl]phenoxy}phenyl)amino]-5H-pyrrolo[3,2-d]pyrimidin-5-yl}ethoxy)ethanol

### (i) Production of [3-(2-chloro-4-nitrophenoxy)phenyl]methanol

To a solution of 3-(hydroxymethyl)phenol (6.21 g) and 3-chloro-4-fluoronitrobenzene (9.24 g) in N,N-dimethylformamide (50 mL) was added potassium carbonate (10.37 g) at room temperature and the mixture was stirred for 4 hr. Under ice-cooling, brine was added to the reaction mixture, and the mixture was extracted with ethyl acetate. The organic layer was dried over anhydrous magnesium sulfate and concentrated under reduced pressure, and the residue was separated and purified by silica gel column chromatography (eluent, ethyl acetate:hexane=20:80→40:60) to give the title compound (5.55 g) as a pale-yellow powder.

¹H-NMR (CDCl₃) δ: 1.78 (1H, t, J=5.7 Hz), 4.74 (2H, d, J=5.7 Hz), 6.90 (1H, d, J=9.0 Hz), 6.95-7.05 (1H, m), 7.12 (1H, s), 7.25-7.30 (1H, m), 7.43 (1H, t, J=8.0 Hz), 8.05-8.10 (1H, m), 8.35-8.40 (1H, m).

### (ii) Production of 1-[3-(2-chloro-4-nitrophenoxy)benzyl]-2-methyl-1H-imidazole

To a solution of [3-(2-chloro-4-nitrophenoxy)phenyl]methanol (1.12 g) in tetrahydrofuran (30 mL) were added triethylamine (0.67 mL) and methanesulfonyl chloride (0.33 mL) under ice-cooling, and the mixture was stirred at 0° C. for 1 hr. Aqueous sodium bicarbonate was added to the reaction mixture and the mixture was extracted with ethyl acetate. The organic layer was washed with brine, dried over anhydrous magnesium sulfate and concentrated under reduced pressure. 2-Methylimidazole (328 mg), potassium carbonate (829 mg) and N,N-dimethylformamide (10 mL), were added to the obtained residue, and the mixture was Stirred at room temperature for 15 hr. Water was added to the reaction mixture and the mixture was extracted with ethyl acetate. The organic layer was washed with brine, dried over anhydrous magnesium sulfate and concentrated under reduced pressure. The residue was separated and purified by basic silica gel column chromatography (eluent, ethyl acetate) to give the title compound (0.96 g) as a pale-yellow oil.

¹H-NMR (CDCl₃) δ: 2.35 (3H, s), 5.08 (2H, s), 6.75-6.9 (3H, m), 6.90-7.05 (3H, m), 7.41 (1H, t, J=7.8 Hz), 8.06 (1H, dd, J=2.7 Hz, 9.0 Hz), 8.38 (1H, d, J=2.7 Hz).

### (iii) Production of 3-chloro-4-{3-[(2-methyl-1H-imidazol-1-yl)methyl]phenoxy}aniline

To a solution of 1-[3-(2-chloro-4-nitrophenoxy)benzyl]-2-methyl-1H-imidazole (0.96 g) in methanol (10 mL) was added 5% platinum-activated carbon (192 mg) under a nitrogen atmosphere. The reaction mixture was stirred under a hydrogen atmosphere at room temperature for 19 hr, and 5% platinum-activated carbon was filtered off. The filtrate was concentrated under reduced pressure and the residue was separated and purified by basic silica gel column chromatography (eluent, ethyl acetate:hexane=60:40→100:0) to give the title compound (494 mg) as a white powder.

¹H-NMR (CDCl₃) δ: 2.32 (3H, s), 3.69 (2H, br s), 4.99 (2H, s), 6.56 (1H, dd, J=2.7 Hz, 9.0 Hz), 6.60-6.70 (2H, m), 6.70-6.85 (3H, m), 6.87 (1H, d, J=9.0 Hz), 6.93 (1H, d, J=1.2 Hz), 7.22 (1H, t, J=7.6 Hz).

### (iv) Production of 2-(2-{4-[(3-chloro-4-{3-[(2-methyl-1H-imidazol-1-yl)methyl]phenoxy}phenyl)amino]-5H-pyrrolo[3,2-d]pyrimidin-5-yl}ethoxy)ethanol

A mixture of 2-[2-(4-chloro-5H-pyrrolo[3,2-d]pyrimidin-5-yl)ethoxy]ethyl benzoate (207 mg), 3-chloro-4-{3-[(2-methyl-1H-imidazol-1-yl)methyl]phenoxy}aniline (154 mg), 1-methyl-2-pyrrolidone (5.0 mL) and pyridine hydrochloride (139 mg) was stirred at 120° C. for 22 hr. Aqueous sodium bicarbonate was added to the reaction mixture and the mixture was extracted with ethyl acetate. The organic layer was washed with brine, dried over anhydrous magnesium sulfate and concentrated under reduced pressure. The residue was separated and purified by basic silica gel column chromatography (eluent, ethyl acetate:methanol=100:0→95:5). To the obtained compound were added 1N aqueous sodium hydroxide solution (2.3 mL) and tetrahydrofuran (4 mL) and the mixture was stirred at room temperature for 21 hr. The reaction mixture was neutralized with 1N hydrochloric acid and aqueous sodium bicarbonate and brine were added. The mixture was extracted with ethyl acetate, and the organic layer was dried over anhydrous magnesium sulfate and concentrated under reduced pressure. The residue was separated and purified by silica gel column chromatography (eluent, ethyl acetate:methanol=100:0→95:5) to give the title compound (119 mg) as a pale-yellow powder.

¹H-NMR (CDCl₃) δ: 2.21 (3H, s), 3.70-3.85 (4H, m), 4.02 (2H, t, J=4.2 Hz), 4.57 (2H, t, J=4.2 Hz), 5.01 (2H, s), 5.99 (1H, s), 6.63 (1H, d, J=3.3 Hz), 6.75-6.85 (1H, m), 6.83 (1H, d, J=8.1 Hz), 6.92 (1H, d, J=9.0 Hz), 6.95-7.05 (1H, m), 7.21 (1H, d, J=3.0 Hz), 7.25-7.30 (1H, m), 7.32 (1H, t, J=8.0 Hz), 7.65 (1H, dd, J=2.4 Hz, 9.0 Hz), 8.02 (1H, d, J=2.4 Hz), 8.54 (1H, s), 8.98 (1H, s).

### Example E-2

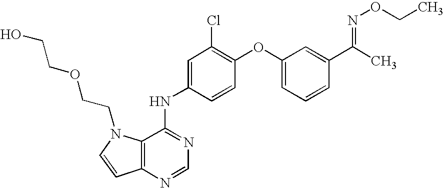

### Production of (1E)-1-{3-[2-chloro-4-({5-[2-(2-hydroxyethoxy)ethyl]-5H-pyrrolo[3,2-d]pyrimidin-4-yl}amino)phenoxy]phenyl}ethanone O-ethyloxime

### (i) Production of (1E)-1-[3-(4-amino-2-chlorophenoxy)phenyl]ethanone O-ethyloxime

To a solution of 1-[3-(4-amino-2-chlorophenoxy)phenyl]ethanone (1.31 g) in ethanol (50 mL) were added O-ethylhydroxylamine hydrochloride (2.44 g) and sodium acetate (2.05 g) and the mixture was stirred at room temperature overnight. The reaction mixture was concentrated under reduced pressure, water was added thereto, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=20:80→40:60). The objective fractions were concentrated under reduced pressure to give the title compound (1.54 g) was obtained as an orange oil.

¹H-NMR (CDCl₃) δ: 1.31 (3H, t, J=7.0 Hz), 2.19 (3H, s), 3.66 (2H, br s), 4.22 (2H, q, J=7.0 Hz), 6.56 (1H, dd, J=2.8 Hz, 8.7 Hz), 6.77-6.83 (2H, m), 6.89 (1H, d, J=8.7 Hz), 7.21-7.28 (2H, m), 7.29-7.35 (1H, m).

### (ii) Production of (1E)-1-{3-[2⁻chloro-4-({5-[2-(2-hydroxyethoxy)ethyl]-5H-pyrrolo[3,2-d]pyrimidin-4-yl}amino)phenoxy]phenyl}ethanone O-ethyloxime

A mixture of 2-[2-(4-chloro-5H-pyrrolo[3,2-d]pyrimidin-5-yl)ethoxy]ethyl benzoate (173 mg), (1E)-1-[3-(4-amino-2-chlorophenoxy)phenyl]ethanone O-ethyloxime (153 mg) and isopropyl alcohol (3 mL) was stirred at 80° C. overnight. An aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure. To the residue were added methanol (5 mL), tetrahydrofuran (1 mL) and 1N aqueous sodium hydroxide solution (1 mL), and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated under reduced pressure. The residue was crystallized from ethyl acetate-diisopropyl ether to give the title compound (134 mg) as a white powder.

¹H-NMR (CDCl₃) δ: 1.32 (3H, t, J=7.1 Hz), 1.88-2.04 (1H, m), 2.21 (3H, s), 3.70-3.82 (4H, m), 3.99-4.05 (2H, m), 4.24 (2H, q, J=7.1 Hz), 4.53-4.59 (2H, m), 6.61 (1H, d, J=3.0 Hz), 6.86-6.92 (1H, m), 7.01 (1H, d, J=8.9 Hz), 7.20 (1H, d, J=3.0 Hz), 7.27-7.40 (3H, m), 7.56 (1H, dd, J=2.7 Hz, 8.9 Hz), 7.87 (1H, d, J=2.7 Hz), 8.51 (1H, s), 8.75 (1H, br s).

### Example E-3

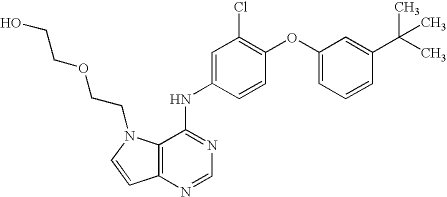

### Production of 2-[2-(4-{[4-(3-tert-butylphenoxy)-3-chlorophenyl]amino}-5H-pyrrolo[3,2-d]pyrimidin-5-yl)ethoxy]ethanol

Using 2-[2-(4-chloro-5H-pyrrolo[3,2-d]pyrimidin-5-yl)ethoxy]ethyl benzoate (173 mg), 4-(3-tert-butylphenoxy)-3-chloroaniline (138 mg), isopropyl alcohol (3 mL), methanol (5 mL), tetrahydrofuran (1 mL) and 1N aqueous sodium hydroxide solution (1 mL) and in the same manner as in Example E-2(ii), the title compound (189 mg) was obtained as a white powder.

¹H-NMR (CDCl₃) δ: 1.32 (9H, s), 1.95-2.30 (1H, m), 3.70-3.82 (4H, m), 3.99-4.05 (2H, m), 4.52-4.59 (2H, m), 6.60 (1H, d, J=3.0 Hz), 6.68-6.74 (1H, m), 6.99 (1H, d, J=8.9 Hz), 7.08-7.15 (2H, m), 7.14-7.28 (2H, m), 7.54 (1H, dd, J=2.5 Hz, 8.9 Hz), 7.86 (1H, d, J=2.5 Hz), 8.49 (1H, s), 8.73 (1H, br s).

### Example E-4

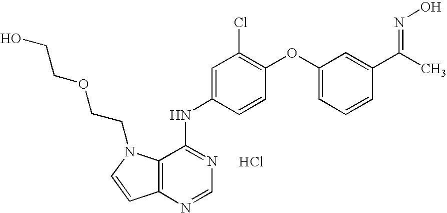

### Production of (1E)-1-{3-[2-chloro-4-({5-[2-(2-hydroxyethoxy)ethyl]-5H-pyrrolo[3,2-d]pyrimidin-4-yl}amino)phenoxy]phenyl}ethanone oxime hydrochloride

### (i) Production of 2-[2-(4-{[4-(3-acetylphenoxy)-3-chlorophenyl]amino}-5H-pyrrolo[3,2-d]pyrimidin-5-yl)ethoxy]ethyl benzoate

A mixture of 2-[2-(4-chloro-5H-pyrrolo[3,2-d]pyrimidin-5-yl)ethoxy]ethyl benzoate (1.04 g), 1-[3-(4-amino-2-chlorophenoxy)phenyl]ethanone (785 mg) and isopropyl alcohol (10 mL) was stirred at 80° C. overnight. An aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure to give the title compound (1.59 g) as a yellow solid.

¹H-NMR (CDCl₃) δ: 2.59 (3H, s), 3.93-3.99 (2H, m), 4.05-4.12 (2H, m), 4.46-4.52 (2H, m), 4.55-4.62 (2H, m), 6.63 (1H, d, J=3.2 Hz), 6.82 (1H, d, J=8.8 Hz), 7.0.7-7.12 (1H, m), 7.22 (1H, d, J=3.2 Hz), 7.29-7.54 (6H, m), 7.63-7.69 (1H, m), 7.75-7.82 (2H, m), 7.89 (1H, d, J=2.7 Hz), 8.50 (1H, s), 8.77 (1H, br s).

### (ii) Production of (1E)-1-{3-[2-chloro-4-({5-[2-(2-hydroxyethoxy)ethyl]-5H-pyrrolo[3,2-d]pyrimidin-4-yl}amino)phenoxy]phenyl}ethanone oxime hydrochloride

To a solution of 2-[2-(4-{[4-(3-acetylphenoxy)-3-chlorophenyl]amino}-5H-pyrrolo[3,2-d]pyrimidin-5-yl)ethoxy]ethyl benzoate (171 mg) in ethanol (5 mL) were added hydroxylamine hydrochloride (104 mg) and sodium acetate (123 mg) and the mixture was stirred at room temperature for 4 hr. Water was added to the reaction mixture, and the mixture was extracted with ethyl acetate. The organic layer was washed with saturated brine and dried over anhydrous magnesium sulfate. The solvent was evaporated under reduced pressure, and methanol (5 mL), tetrahydrofuran (1 mL) and 1N aqueous sodium hydroxide solution (0.6 mL) were added to the obtained residue. The mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→10:90). The objective fractions were concentrated, under reduced pressure. The residue was dissolved in ethyl acetate-ethanol, and 1N hydrogen chloride/ethyl acetate solution (0.3 mL) was added thereto. The solvent was evaporated under reduced pressure and the obtained residue was crystallized from ethanol-ethyl acetate to give the title compound (75 mg) as a white powder.

¹H-NMR (DMSO-d₆) δ: 2.14 (3H, s), 3.40-3.54 (4H, m), 3.81-3.89 (2H, m), 4.76-4.85 (2H, m), 6.69 (1H, d, J=3.0 Hz), 7.00-7.06 (1H, m), 7.22 (1H, m), 7.27 (1H, d, J=8.7 Hz), 7.37-7.48 (2H, m), 7.62 (1H, dd, J=2.5 Hz, 8.7 Hz), 7.96 (1H, d, J=2.5 Hz), 8.01 (1H, m), 8.73 (1H, s), 9.80-9.90 (1H, m), 11.31 (1H, s).

### Example E-5

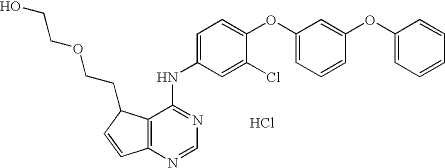

### Production of 2-[2-(4-{[3-chloro-4-(3-phenoxyphenoxy)phenyl]amino}-5H-pyrrolo[3,2-d]pyrimidin-5-yl)ethoxy]ethanol hydrochloride

### (i) Production of 2-chloro-4-nitro-1-(3-phenoxyphenoxy)benzene

2-Chloro-1-fluoro-4-nitrobenzene (0.943 g) and 3-phenoxyphenol (1 g) were dissolved in N,N-dimethylformamide (5.4 mL), and potassium carbonate (1.07 g) was added thereto. The mixture was stirred at room temperature for 16 hr. The reaction mixture was diluted with ethyl acetate (80 mL), and washed with water (70 mL). The organic layer was dried over anhydrous magnesium sulfate and concentrated under reduced pressure. The residue was subjected to silica gel column chromatography (hexane/ethyl acetate=100/0→70/30) to give the title compound (1.75 g) as an oil.

¹H-NMR (CDCl₃) δ: 6.73 (1H, t, J=2 Hz), 6.78 (1H, m), 6.89 (1H, m), 6.95 (2H, d, J=9 Hz), 7.05 (2H,m), 7.16 (1H, m), 7.37 (3H, m), 8.07 (1H, dd, J=3 Hz, 9 Hz), 8.37 (1H, d, J=3 Hz).

### (ii) Production of 3-chloro-4-(3-phenoxyphenoxy)aniline

2-Chloro-4-nitro-1-(3-phenoxyphenoxy)benzene (1.7 g) was suspended in ethanol (49 mL)/water (5.45 mL), calcium chloride (306 mg) was added thereto and the mixture was dissolved by heating with stirring at 90° C. for 10 min. Reduced iron (1.85 g) was added and the mixture was stirred with heating at 90° C. for 16 hr. After cooling to room temperature, the reaction mixture was filtered through celite, and the filtrate was concentrated under reduced pressure. The residual solid was diluted with ethyl acetate (200 mL), and washed with saturated brine (100 mL). The organic layer was dried over anhydrous magnesium sulfate and concentrated under reduced pressure. The residue was purified by silica gel chromatography (hexane/ethyl acetate=80/20→60/40) to give the title compound (1.58 g) as an oil.

¹H-NMR (CDCl₃) δ: 3.67 (2H, br s), 6.50-6.70 (4H, m), 6.76 (1H, d, J=3 Hz), 6.92 (1H, d, J=9 Hz), 7.01 (2H, m), 7.10 (1H, m), 7.20 (1H, m), 7.33 (2H, m).

### (iii) Production of 2-[2-(4-{[3-chloro-4-(3-phenoxyphenoxy)phenyl]amino}-5H-pyrrolo[3,2-d]pyrimidin-5-yl)ethoxy]ethanol hydrochloride

A mixture of 2-[2-(4-chloro-5H-pyrrolo[3,2-d]pyrimidin-5-yl)ethoxy]ethyl benzoate (150 mg), 3-chloro-4-(3-phenoxyphenoxy)aniline (201 mg) and 1-methyl-2-pyrrolidone (0.863 mL) was stirred with heating at 140° C. for 3 hr. The reaction mixture was diluted with ethyl acetate (80 mL), and washed with aqueous sodium bicarbonate (30 mL). The organic layer was dried over anhydrous magnesium sulfate, and concentrated under is reduced pressure. The residue was purified by silica gel column chromatography (hexane:ethyl acetate=90:10→0:100) and the objective fractions were concentrated under reduced pressure. The obtained residue was dissolved in methanol (1.89 mL), 1N aqueous sodium hydroxide solution (0.433 mL) was added thereto, and the mixture was stirred at room temperature for 2 hr. 1N hydrochloric acid (0.433 mL) was added, and the mixture was diluted with ethyl acetate (80 mL), and washed with saturated brine (30 mL). The organic layer was dried over anhydrous magnesium sulfate and concentrated under reduced pressure. The residue was subjected to silica gel column chromatography (ethyl acetate:methanol=100:0→85:15). The fraction containing the title compound was concentrated under reduced pressure, and the residue was dissolved in ethyl acetate (4 mL). 4N hydrochloric acid (0.125 mL) was added to the mixture, and the mixture was crystallized from isopropyl ether/ethyl acetate to give the title compound (129 mg).

¹H-NMR (DMSO-d₆) δ: 3.46 (4H, d, J=2 Hz), 3.84 (2H, br s), 4.85 (2H, br s), 6.60 (1H, s), 7.07 (2H, d, J=8 Hz), 7.18 (2H, t, J=8 Hz), 7.29 (1H, d, J=8 Hz), 7.30-7.50 (3H, m), 7.64 (1H, d, J=9 Hz), 7.96 (1H, d, J=1 Hz), 8.04 (1H, d, J=3 Hz), 8.74 (1H, s), 10.02 (1H, br s).

### Example E-6

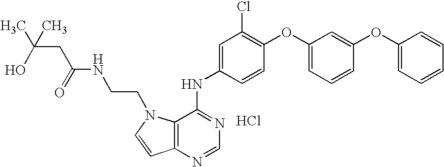

### Production of N-[2-(4-{[3-chloro-4-(3-phenoxyphenoxy)phenyl]amino}-5H-pyrrolo[3,2-d]pyrimidin-5-yl)ethyl]-3-hydroxy-3-methylbutanamide hydrochloride

### (i) Production of tert-butyl[2-(4-{[3-chloro-4-(3-phenoxyphenoxy)phenyl]amino}-5H-pyrrolo[3,2-d]pyrimidin-5-yl)ethyl]carbamate

A solution of tert-butyl[2-(4-chloro-5H-pyrrolo[3,2-d]pyrimidin-5-yl)ethyl]carbamate (0.5 g) and 3-chloro-4-(3-phenoxyphenoxy)aniline (786 mg) in isopropyl alcohol (5 mL) was stirred at 80° C. for 12 hr. Aqueous sodium bicarbonate (30 mL) was added to the reaction mixture, and the mixture was extracted with ethyl acetate (80 mL). The organic layer was washed with saturated brine and dried over anhydrous magnesium sulfate. After concentration under reduced pressure, the residue was separated and purified by silica gel column chromatography (eluent, hexane:ethyl acetate=7:3→ethyl acetate) to give the title compound (713 mg) as colorless crystals.

¹H-NMR (CDCl₃) δ: 1.48 (9H, s), 3.48 (2H, m), 4.46 (2H,m), 5.12 (1H, t, J=5 Hz), 6.59 (1H, d, J=3 Hz), 6.69 (3H, m), 7.00-7.40 (8H, m), 7.85 (1H, dd, J=3 Hz, 9 Hz), 7.98 (1H, d, J=3 Hz), 8.50 (1H, s), 8.58 (1H, br s).

### (ii) Production of 5-(2-aminoethyl)-N-[3-chloro-4-(3-phenoxyphenoxy)phenyl]-5H-pyrrolo[3,2-d]pyrimidin-4-amine dihydrochloride

A mixture of tert-butyl[2-(4-{[3-chloro-4-(3-phenoxyphenoxy)phenyl]amino}-5H-pyrrolo[3,2-d]pyrimidin-5-yl)ethyl]carbamate (683 mg), 2N hydrochloric acid (8.46 mL) and tetrahydrofuran (16.9 mL) was stirred at 60° C. for 20 hr. The solvent was evaporated under reduced pressure, ethanol was added, and the mixture was further concentrated. The precipitated powder was collected by filtration. The powder was washed with isopropyl ether to give the title compound (622 mg) as a pale-yellow powder.

¹H-NMR (DMSO-d₆) δ: 3.30 (2H, m), 5.08 (2H, m), 6.60 (1H, t, J=2 Hz), 6.73 (3H, m), 7.06 (2H, m), 7.18 (1H, m), 7.29 (1H, d, J=9 Hz), 7.41 (3H, m), 7.64 (1H, dd, J=3 Hz, 9 Hz), 7.90 (1H, d, J=3 Hz); 8.10 (1H, d, J=3 Hz), 8.42 (3H, br s), 8.73 (1H, s).

### (iii) Production of N-[2-(4-{[3-chloro-4-(3-phenoxyphenoxy)phenyl]amino}-5H-pyrrolo[3,2-d]pyrimidin-5-yl)ethyl]-3-hydroxy-3-methylbutanamide hydrochloride

A mixture of 5-(2-aminoethyl)-N-[3-chloro-4-(3-phenoxyphenoxy)phenyl]-5H-pyrrolo[3,2-d]pyrimidin-4-amine dihydrochloride (195 mg), 3-hydroxy-3-methylbutanoic acid (0.058 mL), 1-ethyl-3-(3-dimethylaminopropyl)carbodiimide hydrochloride (103 mg), 1-hydroxybenzotriazole (72.5 mg), triethylamine (0.15 mL) and N,N-dimethylformamide (6.9 mL) was stirred at room temperature for 16 hr. Water was added to the reaction mixture and the mixture was extracted with ethyl acetate. The organic layer was washed successively with water and saturated brine, and dried over anhydrous magnesium sulfate. After concentration under reduced pressure, the residue was separated and purified by basic silica gel column chromatography (eluent, ethyl acetate→ethyl acetate:methanol=85:15). The fraction containing the title compound was concentrated under reduced pressure, and the residue was dissolved in ethyl acetate (4 mL). 4N hydrochloric acid (0.169 mL) was added, and the mixture was crystallized from ethyl acetate to give the title compound (176 mg).

¹H-NMR (DMSO-d₆) δ: 1.10 (6H, s), 2.19 (2H, s), 3.48 (2H, q, J=6 Hz), 4.66 (2H, t, J=6 Hz), 6.60 (1H, t, J=3 Hz), 6.67 (1H, d, J=3 Hz), 6.72 (2H, dt, J=3 Hz, 9 Hz), 7.06 (2H, d, J=8 Hz), 7.17 (1H, t, J=7 Hz), 7.29 (1H, d, J=9 Hz), 7.40 (3H, m), 7.67 (1H, dd, J=3 Hz, 9 Hz), 7.92 (1H, d, J=3 Hz), 7.98 (1H, d, J=3 Hz), 8.40 (1H, m), 8.71 (1H, s).

### Example E-7

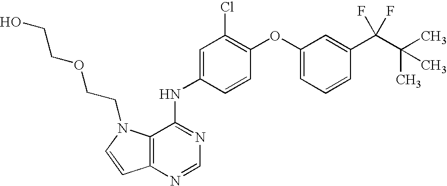

### Production of 2-{2-[4-({3-chloro-4-[3-(1,1-difluoro-2,2-dimethylpropyl)phenoxy]phenyl}amino)-5H-pyrrolo[3,2-d]pyrimidin-5-yl]ethoxy}ethanol

### (i) Production of 1-[3-(2-chloro-4-nitrophenoxy)phenyl]-2,2-dimethylpropan-1-one

A mixture of 2-chloro-1-fluoro-4-nitrobenzene (2.63 g), 1-(3-hydroxyphenyl)-2,2-dimethylpropan-1-one (2.55 g), potassium carbonate (2.97 g) and N,N-dimethylformamide (20 mL) was stirred at room temperature overnight. The reaction mixture was concentrated under reduced pressure, water was added and the mixture was extracted with ethyl acetate. The organic layer was washed with saturated brine, and dried over anhydrous magnesium sulfate. The solvent was evaporated under reduced pressure and the obtained residue was subjected to silica gel column chromatography (eluent, ethyl acetate:hexane=2:98→15:85). The objective fractions were concentrated under reduced pressure to give the title compound (5.17 g) as a pale-yellow-green oil.

¹H-NMR (CDCl₃) δ: 1.35 (9H, s), 6.92 (1H, d, J=9.1 Hz), 7.18 (1H, ddd, J=1.1 Hz, 2.6 Hz, 8.1 Hz), 7.37 (1H, m), 7.45-7.52 (1H, m), 7.56-7.60 (1H, m), 8.08 (1H, dd, J=2.7 Hz, 9.1 Hz), 8.40 (1H, d, J=2.7 Hz).

### (ii) Production of 2-chloro-1-[3-(1,1-difluoro-2,2-dimethylpropyl)phenoxy]-4-nitrobenzene

A solution of 1-[3-(2-chloro-4-nitrophenoxy)phenyl]-2,2-dimethylpropan-1-one (3.34 g) and diethylaminosulfur trifluoride (685 g) in dichloromethane (100 mL) was stirred with heating under reflux overnight. Ice and aqueous sodium hydrogencarbonate solution were added to the reaction mixture, and the mixture was extracted with dichloromethane. The dichloromethane layer was washed with saturated brine, and dried over anhydrous sodium sulfate. The solvent was evaporated under reduced pressure and the obtained residue was subjected to silica gel column chromatography (eluent, ethyl acetate:hexane=2:98→15:85). The objective fractions were concentrated under reduced pressure to give the title compound (3.63 g) as a colorless oil.

¹H-NMR (CDCl₃) δ: 1.05 (9H, s), 6.87 (1H, d, J=9.1 Hz), 7.11-7.18 (2H, m), 7.31-7.37 (1H, m), 7.44-7.52 (1H, m), 8.07 (1H, dd, J=2.7 Hz, 9.1 Hz), 8.40 (1H, d, J=2.7 Hz).

### (iii) Production of 3-chloro-4-[3-(1,1-difluoro-2,2-dimethylpropyl)phenoxy]aniline

A mixture of 2-chloro-1-[3-(1,1-difluoro-2,2-dimethylpropyl)phenoxy]-4-nitrobenzene (712 mg), reduced iron (372 mg), calcium chloride (123 mg) and 10% water-containing ethanol (20 mL) was stirred with heating under reflux for 7 hr. Separately, a mixture of 2-chloro-1-[3-(1,1-difluoro-2,2-dimethylpropyl)phenoxy]-4-nitrobenzene (3.00 g), reduced iron (1.57 g), calcium chloride (520 mg) and 10% water-containing ethanol (100 mL) were stirred with heating under reflux overnight. They were combined and the solid was removed by filtration. The filtrate was concentrated and water was added to the obtained residue, and the mixture was extracted with ethyl acetate. The ethyl acetate layer was washed with saturated brine and dried over anhydrous magnesium sulfate. The solvent was evaporated under reduced pressure and the obtained residue was subjected to silica gel column chromatography (eluent, ethyl acetate:hexane=15:85→30:70). The objective fractions were concentrated under reduced pressure to give the title compound (2.40 g) as a pale-brown solid.

¹H-NMR (CDCl₃) δ: 1.02 (9H, s), 3.68 (2H, br s), 6.57 (1H, dd, J=2.7 Hz; 8.8 Hz), 6.79 (1H, d, J=2.7 Hz), 6.87-6.95 (3H, m), 7.05-7.11 (1H, m), 7.28-7.33 (1H, m

### (iv) Production of 2-{2-[4-({3-chloro-4-[3-(1,1-difluoro-2,2-dimethylpropyl)phenoxy]phenyl}amino)-5H-pyrrolo[3,2-d]pyrimidin-5-yl]ethoxy}ethanol

Using 2-[2-(4-chloro-5H-pyrrolo[3,2-d]pyrimidin-5-yl)ethoxy]ethyl benzoate (173 mg), 3-chloro-4-[3-(1,1-difluoro-2,2-dimethylpropyl)phenoxy]aniline (163 mg), isopropyl alcohol (3 mL), methanol (5 mL), tetrahydrofuran (1 mL) and 1N aqueous sodium hydroxide solution (1 mL) and in the same manner as in Example E-2(ii), the title compound (198 mg) was obtained as a white powder.

¹H-NMR (CDCl₃) δ: 1.04 (9H, s), 2.05 (1H, br s), 3.70-3.84 (4H, m), 4.02 (2H, t, J=4.3 Hz), 4.56 (2H, t, J=4.3 Hz), 6.61 (1H, d, J=3.2 Hz), 6.94-7.08 (3H, m), 7.13 (1H, d, J=7.7 Hz), 7.20 (1H, d, J=3.2 Hz), 7.32 (1H, t, J=8.0 Hz), 7.58 (1H, dd, J=2.6 Hz, 8.9 Hz), 7.89 (1H, d, J=2.6 Hz), 8.51 (1H, s), 8.78 (1H, br s).

### Example E-8

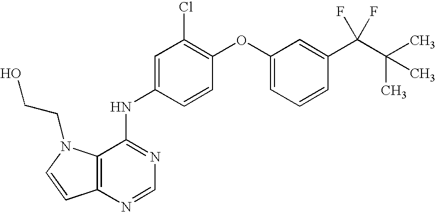

### Production of 2-[4-({3-chloro-4-[3-(1,1-difluoro-2,2-dimethylpropyl)phenoxy]phenyl}amino)-5H-pyrrolo-[3,2-d]pyrimidin-5-yl]ethanol

A mixture of 2-(4-chloro-5H-pyrrolo[3,2-d]pyrimidin-5-yl)ethyl benzoate (151 mg), 3-chloro-4-[3-(1,1-difluoro-2,2-dimethylpropyl)phenoxy]aniline (163 mg) and isopropyl alcohol (3 mL) was stirred at 80° C. for 9 hr. An aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=60:40→100:0). The objective fractions were concentrated under reduced pressure. To the residue were added methanol (5 mL), tetrahydrofuran (1 mL) and 1N aqueous sodium hydroxide solution (1 mL) and the mixture was stirred at room temperature for 4 hr. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, methanol:ethyl acetate=0:100→10:90). The objective fractions were concentrated under reduced pressure. The residue was crystallized from ethyl acetate-diisopropyl ether to give the title compound (191 mg) as a white powder.

¹H-NMR (CDCl₃) δ: 1.05 (9H, s), 4.11-4.18 (2H, m), 4.33-4.40 (2H, m), 6.69 (1H, d, J=3.3 Hz), 6.50 (1H, br s), 6.95 (1H, d, J=3.3 Hz), 6.97-7.08 (3H, m), 7.14 (1H, d, J=7.7 Hz), 7.34 (1H, t, J=8.0 Hz), 7.47 (1H, dd, J=2.5 Hz, 8.8 Hz), 7.82 (1H, d, J=2.5 Hz), 8.21 (1H, s), 9.56 (1H, br s).

### Example E-9

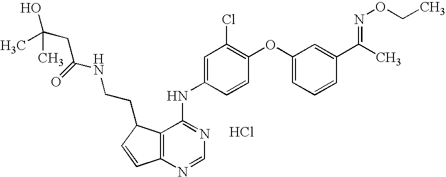

### Production of N-(2-{4-[(3-chloro-4-{3-[(1E)-N-ethoxyacetoimidoyl]phenoxy}phenyl)amino]-5H-pyrrolo[3,2-d]pyrimidin-5-yl}ethyl)-3-hydroxy-3-methylbutanamide hydrochloride

### (i) Production of tert-butyl[2-(4-{[4-(3-acetylphenoxy)-3-chlorophenyl]amino}-5H-pyrrolo[3,2-d]pyrimidin-5-yl)ethyl]carbamate

A mixture of tert-butyl[2-(4-chloro-5H-pyrrolo[3,2-d]pyrimidin-5-yl)ethyl]carbamate (890 mg), 1-[3-(4-amino-2-chlorophenoxy)phenyl]ethanone (785 mg) and isopropyl alcohol (10 mL) was stirred at 80° C. overnight. An aqueous sodium hydrogencarbonate solution was added to the reaction mixture, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=50:50→100:0). The objective fractions were concentrated under reduced pressure and the residue was crystallized from acetone-diethyl ether to give the title compound (1.14 g) as a pale-yellow powder.

¹H-NMR (CDCl₃) δ: 1.50 (9H, s), 2.59 (3H, m), 3.44-3.55 (2H, m), 4.44-4.53 (2H, m), 5.08 (1H, t, J=5.6 Hz), 6.61 (1H, d, J=3.3 Hz), 7.05 (1H, d, J=8.9 Hz), 7.15-7.21 (2H, m), 7.4.1 (1H, t, J=8.0 Hz), 7.56 (1H, m), 7.66 (1H, d, J=7.7 Hz), 7.89 (1H, dd, J=2.7 Hz, 8.9 Hz), 8.03 (1H, d, J=2.7 Hz), 8.52 (1H, s), 8.60 (1H, br s).

### (ii) Production of N-[2-(4-{[4-(3-acetylphenoxy)-3-chlorophenyl]amino}-5H-pyrrolo[3,2-d]pyrimidin-5-yl)ethyl]-3-hydroxy-3-methylbutanamide

To tert-butyl[2-(4-{[4-(3-acetylphenoxy)-3-chlorophenyl]amino}-5H-pyrrolo[3,2-d]pyrimidin-5-yl)ethyl]carbamate (1.10 g) were added ethanol (2 mL) and 4N hydrogen chloride/ethyl acetate solution (5 mL) and the mixture was stirred at room temperature overnight. The reaction mixture was concentrated under reduced pressure. To a solution of the obtained residue in N,N-dimethylformamide (20 mL) were added 3-hydroxy-3-methylbutanoic acid (374 mg), 1-ethyl-3-(3-dimethylaminopropyl)carbodiimide hydrochloride (607 mg), 1-hydroxybenzotriazole monohydrate (485 mg) and triethylamine (0.88.2 mL) and the mixture was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to silica gel column chromatography (eluent, ethyl acetate:hexane=50:50→100:0-4methanol:ethyl acetate=20:80) and basic silica gel column chromatography (eluent, methanol:ethyl acetate=0:100→10:90). The objective fractions were concentrated under reduced pressure to give the title compound (596 mg) as a white amorphous powder.

¹H-NMR (CDCl₃) δ: 1.32 (6H, s), 2.48 (2H, s), 2.59 (3H, s), 3.58-3.68 (2H, m), 4.44-4.54 (2H, m), 6.59 (1H, d, J=3.3 Hz), 7.05 (1H, d, J=8.8 Hz), 7.09-7.22 (3H, m), 7.42 (1H, t, J=7.8 Hz), 7.57 (1H, m), 7.64-7.69 (1H, m), 7.74 (1H, dd, J=2.6 Hz, 8.8 Hz), 8.07 (1H, d, J=2.6 Hz), 8.50 (1H, s), 8.66 (1H, br s).

### (iii) Production of N-(2-{4-[(3-chloro-4-{3-[(1E)-N-ethoxyacetoimidoyl]phenoxy}phenyl)amino]-5H-pyrrolo[3,2-d]pyrimidin-5-yl}ethyl)-3-hydroxy-3-methylbutanamide hydrochloride

To a solution of N-[2-(4-{[4-(3-acetylphenoxy)-3-chlorophenyl]amino}-5H-pyrrolo[3,2-d]pyrimidin-5-yl)ethyl]-3-hydroxy-3-methylbutanamide (157 mg) in ethanol (5 mL) were added O-ethylhydroxylamine hydrochloride (88 mg) and sodium acetate (74 mg) and the mixture was stirred at room temperature overnight. Water was added to the reaction mixture, and the mixture was extracted with ethyl acetate. The organic layer was washed with saturated brine and dried over anhydrous magnesium sulfate. The solvent was evaporated under reduced pressure and the obtained residue was subjected to basic silica gel column chromatography (eluent, methanol:ethyl acetate=0:100→15:85). The objective fractions were concentrated under reduced pressure. The residue was dissolved in ethyl acetate-ethanol, and 1N hydrogen chloride/ethyl acetate solution (0.3 mL) was added. The solvent was evaporated under reduced pressure and the obtained residue was crystallized from ethyl acetate-diethyl ether to give the title compound (91 mg) as a white powder.

¹H-NMR (DMSO-d₆) δ: 1.11 (6H, s), 1.25 (3H, t, J=7.1 Hz), 2.18 (3H, s), 2.20 (2H, s), 3.43-3.55 (2H, m), 4.17 (2H, q, J=7.1 Hz), 4.66 (2H, t, J=6.7 Hz), 6.67 (1H, d, J=3.2 Hz), 6.95-7.02 (1H, m), 7.22 (1H, d, J=8.9 Hz), 7.33 (1H, m), 7.40-7.49 (2H, m), 7.67 (1H, dd, J=J2.5 Hz, 8.9 Hz), 7.94 (1H, d, J=2.5 Hz), 7.98 (1H, d, J=3.2 Hz), 8.40 (1H, t, J=5.6 Hz), 8.72 (1H, s), 10.28 (1H, br s).

### Example E-10

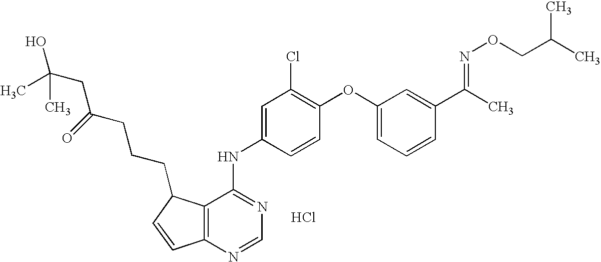

### Production of N-(2-{4-[(3-chloro-4-{3-[(1E)-N-isobutoxyacetoimidoyl]phenoxy}phenyl)amino]-5H-pyrrolo[3,2-d]pyrimidin-5-yl}ethyl)-3-hydroxy-3-methylbutanamide hydrochloride

Using N-[2-(4-{[4-(3-acetylphenoxy)-3-chlorophenyl]amino}-5H-pyrrolo[3,2-d]pyrimidin-5-yl)ethyl]-3-hydroxy-3-methylbutanamide (157 mg), ethanol (5 mL), O-isobutylhydroxylamine hydrochloride (113 mg), sodium acetate (74 mg) and 1N hydrogen chloride/ethyl acetate solution (0.3 mL) and in the same manner as in Example E-9(iii), the title compound (87 mg) was obtained as a white powder.

¹H-NMR (DMSO-d₆) δ: 0.92 (6H, d, J=6.9 Hz), 1.11 (6H, s), 1.90-2.05 (1H, m), 2.20 (5H, s), 3.44-3.54 (2H, m), 3.91 (2H, d, J=6.6 Hz), 4.66 (2H, t, J=6.7 Hz), 6.67 (1H, d, J=3.0 Hz), 6.94-7.02 (1H, m), 7.22 (1H, d, J=8.9 Hz), 7.32 (1H, m), 7.40-7.48 (2H, m), 7.67 (1H, dd, J=2.5 Hz, 8.9 Hz), 7.94 (1H, d, J=2.5 Hz), 7.99 (1H, d, J=3.0 Hz), 8.40 (1H, t, J=5.5 Hz), 8.72 (1H, s), 10.29 (1H, br s).

### Example E-11

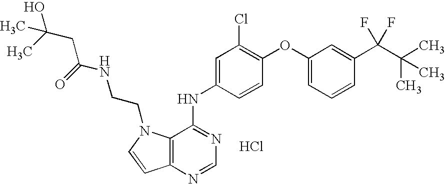

### Production of N-{2-[4-({3-chloro-4-[3-(1,1-difluoro-2,2-dimethylpropyl)phenoxy]phenyl}amino)-5H-pyrrolo[3,2-d]pyrimidin-5-yl]ethyl}-3-hydroxy-3-methylbutanamide hydrochloride

### (i) Production of tert-butyl{2-[4-({3-chloro-4-[3-(1,1-difluoro-2,2-dimethylpropyl)phenoxy]phenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (594 mg), 3-chloro-4-[3-(1,1-difluoro-2,2-dimethylpropyl)phenoxy]aniline (652 mg) and isopropyl alcohol (10 mL) and in the same manner as in Example E-9(i), the title compound (1.15 g) was obtained as a pale-yellow amorphous powder.

¹H-NMR (CDCl₃) δ: 1.04 (9H, s), 1.49 (9H, s), 3.42-3.54 (2H, m), 4.41-4.52 (2H, m), 5.20 (1H, t, J=5.4 Hz), 6.58 (1H, d, J=3.3 Hz), 6.96-7.08 (3H, m), 7.12 (1H, d, J=7.7 Hz), 7.17 (1H, d, J=3.3 Hz), 7.32 (1H, t, J=8.0 Hz), 7.84 (1H, dd, J=2.5 Hz, 8.8 Hz), 8.01 (1H, d, J=2.5 Hz), 8.50 (1H, s), 8.61 (1H, br s).

### (ii) Production of 5-(2-aminoethyl)-N-{3-chloro-4-[3-(1,1-difluoro-2,2-dimethylpropyl)phenoxy]phenyl}-5H-pyrrolo[3,2-d]pyrimidin-4-amine dihydrochloride

To tert-butyl{2-[4-({3-chloro-4-[3-(1,1-difluoro-2,2-dimethylpropyl)phenoxy]phenyl}amino)-5H-pyrrolo[3,2-d]pyrimidin-5-yl]ethyl}carbamate (1.15 g) was added methanol (10 mL) and concentrated hydrochloric acid (2 mL), and the mixture was stirred at room temperature overnight and at 60° C. for 3 hr. The reaction mixture was concentrated under reduced pressure, isopropyl alcohol and methanol were added thereto, and the mixture was concentrated again under reduced pressure. Isopropyl alcohol and diisopropyl ether were added to the residue, and the crystals were collected by filtration to give the title compound (1.09 g) as a white powder.

¹H-NMR (DMSO-d₆) δ: 0.99 (9H, s), 3.24-3.36 (2H, m), 5.01 (2H, t, J=6.1 Hz), 6.73 (1H, d, J=3.1 Hz), 6.93 (1H, m), 7.11 (1H, dd, J=2.4 Hz, 8.0 Hz), 7.19 (1H, d, J=8.1 Hz), 7.27 (1H, d, J=8.9 Hz), 7.51 (1H, t, J=8.0 Hz), 7.62 (1H, dd, J=2.4 Hz, 8.9 Hz), 7.90 (1H, d, J=2.4 Hz), 8.05 (1H, d, J=3.1 Hz), 8.23-8.33 (3H, m), 8.71 (1H, s), 10.06 (1H, br s).

### (iii) Production of N-{2-[4-({3-chloro-4-[3-(1,1-difluoro-2,2-dimethylpropyl)phenoxy]phenyl}amino)-5H-pyrrolo[3,2-d]pyrimidin-5-yl]ethyl}-3-hydroxy-3-methylbutanamide hydrochloride

A mixture of 5-(2-aminoethyl)-N-{3-chloro-4-[3-(1,1-difluoro-2,2-dimethylpropyl)phenoxy]phenyl}-5H-pyrrolo[3,2-d]pyrimidin-4-amine dihydrochloride (168 mg), 3-hydroxy-3-methylbutanoic acid (53 mg), 1-ethyl-3-(3-dimethylaminopropyl)carbodiimide hydrochloride (86 mg), 1-hydroxybenzotriazole monohydrate (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to basic silica gel column chromatography (eluent, methanol:ethyl acetate=0:100→20:80). The objective fractions were concentrated under reduced pressure. The residue was dissolved in ethyl acetate-ethanol, and 1N hydrogen chloride/ethyl acetate solution (0.3 mL) was added. The solvent was evaporated under reduced pressure and the obtained residue was crystallized from ethyl acetate to give the title compound (134 mg) as a white powder.

¹H-NMR (DMSO-d₆) δ: 0.99 (9H, s), 1.11 (6H, s), 2.20 (2H, s), 3.44-3.54 (2H, m), 4.66 (2H, t, J=7.0 Hz), 6.67 (1H, d, J=3.2 Hz), 6.92 (1H, m), 7.13 (1H, dd, J=2.3 Hz, 8.1 Hz), 7.20 (1H, d, J=7.7 Hz), 7.29 (1H, d, J=8.9 Hz), 7.52 (1H, t, J=8.0 Hz), 7.70 (1H, dd, J=2.5 Hz, 8.9 Hz), 7.96 (1H, d, J=2.5 Hz), 7.99 (1H, d, J=3.2 Hz), 8.41 (1H, t, J=5.1 Hz), 8.73 (1H, s), 10.28 (1H, br s).

### Example E-12

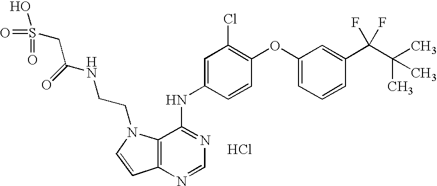

### Production of N-{2-[4-({3-chloro-4-[3-(1,1-difluoro-2,2-dimethylpropyl)phenoxy]phenyl}amino)-5H-pyrrolo[3,2-d]pyrimidin-5-yl]ethyl}-2-(methylsulfonyl)acetamide

A mixture of 5-(2-aminoethyl)-N-{3-chloro-4-[3-(1,1-difluoro-2,2-dimethylpropyl)phenoxy]phenyl}-5H-pyrrolo[3,2-d]pyrimidin-4-amine dihydrochloride (168 mg), methylsulfonylacetic acid (62 mg), 1-ethyl-3-(3-dimethylaminopropyl)carbodiimide hydrochloride (86 mg), 1-hydroxybenzotriazole monohydrate (69 mg), triethylamine (0.100 mL) and N,N-dimethylformamide (3 mL) was stirred at room temperature overnight. Water was added to the reaction mixture, and the mixture was extracted with ethyl acetate. The organic layer was washed successively with water and saturated brine, and dried over anhydrous magnesium sulfate. The solvent was evaporated under reduced pressure and the obtained residue was subjected to basic silica gel column chromatography (eluent, methanol:ethyl acetate=0:100→20:80). The objective fractions were concentrated under reduced pressure. The residue was crystallized from ethyl acetate-diisopropyl ether to give the title compound (153 mg) as a white powder.

¹H-NMR (CDCl₃) δ: 1.04 (9H, s), 3.14 (3H, s), 3.65-3.75 (2H, m), 3.98 (2H, s), 4.44-4.53 (2H, m), 6.59 (1H, d, J=3.0 Hz), 6.98-7.04 (2H, m), 7.07 (1H, m), 7.15 (1H, d, J=7.7 Hz), 7.21 (1H, d, J=3.0 Hz), 7.34 (1H, d, J=8.0 Hz), 7.62 (1H, t, J=5.5 Hz), 7.72 (1H, dd, J=2.5 Hz, 8.9 Hz), 7.94 (1H, d, J=2.5 Hz), 8.18 (1H, br s), 8.50 (1H, s).

### Example E-13

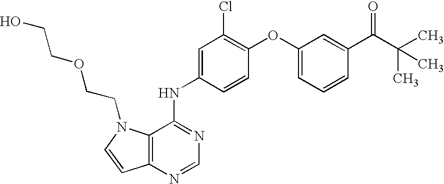

### Production of 1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one

### (i) Production of 1-[3-(4-amino-2-chlorophenoxy)phenyl]-2,2-dimethylpropan-1-one

A mixture of 1-[3-(2-chloro-4-nitrophenoxy)phenyl]-2,2-dimethylpropan-1-one (1.03 g), reduced iron (575 mg), calcium chloride (190 mg) and 10% water-containing ethanol (30 mL) was stirred with heating under reflux overnight. The reaction mixture was filtered to remove solid, and the filtrate was concentrated. Water was added to the residue, and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, ethyl acetate:hexane=20:80→40:60). The objective fractions were concentrated under reduced pressure to give the title compound (708 mg) as a brown oil.

¹H-NMR (CDCl₃) δ: 1.31 (9H, s), 3.69 (2H, br s), 6.58 (1H, dd, J=2.7 Hz, 8.5 Hz), 6.79 (1H, d, J=2.7 Hz), 6.91 (1H, d, J=8.5 Hz), 6.94-7.00 (1H, m), 7.16 (1H, m), 7.26-7.35 (2H, m).

### (ii) Production of 1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one

Using 2-[2-(4-chloro-5H-pyrrolo[3,2-d]pyrimidin-5-yl)ethoxy]ethyl benzoate (734 mg), 1-[3-(4-amino-2-chlorophenoxy)phenyl]-2,2-dimethylpropan-1-one (645 mg), isopropyl alcohol (10 mL), methanol (25 mL), tetrahydrofuran (5 mL) and 1N aqueous sodium hydroxide solution (5 mL) and in the same manner as in Example E-2(ii), the title compound (858 mg) was obtained as a white powder.

¹H-NMR (CDCl₃) δ: 1.33 (9H, s), 3.70-3.82 (4H, m), 4.02 (2H, t, J=4.4 Hz), 4.55 (2H, J=4.4 Hz), 6.58 (1H, d, J=3.3 Hz), 7.00-7.06 (2H, m), 7.19 (1H, d, J=3.3 Hz), 7.22 (1H, m), 7.28-7.39 (2H, m), 7.57 (1H, dd, J=2.6 Hz, 8.8 Hz), 7.89 (1H, d, J=2.6 Hz), 8.48 (1H, s), 8.81 (1H, br s).

### Example E-14

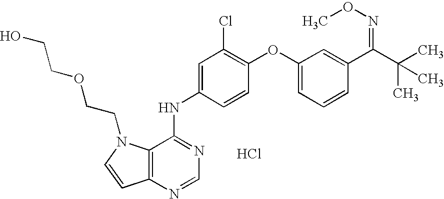

### Production of (1Z)-1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one O-methyloxime hydrochloride

Using 1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one (204 mg), ethanol (5 mL), O-methylhydroxylamine hydrochloride (100 mg), sodium acetate (98 mg) and 1N hydrogen chloride/ethyl acetate solution (0.4 mL) and in the same manner as in Example E-9(iii), the title compound (201 mg) was obtained as a white powder.

¹H-NMR (DMSO-d₆) δ: 1.10 (9H, s), 3.41-3.51 (4H, m), 3.65 (3H, s), 3.84 (2H, t, J=4.4 Hz), 4.78-4.85 (2H, m), 6.62 (1H, m), 6.69 (1H, d, J=3.0 Hz), 6.82 (1H, d, J=7.7 Hz), 6.94 (1H, dd, J=2.5 Hz, 8.0 Hz), 7.22 (1H, d, J=8.9 Hz), 7.42 (1H, t, J=8.0 Hz), 7.61 (1H, dd, J=2.5 Hz, 8.9 Hz), 7.95 (1H, d, J=2.5 Hz), 8.02 (1H, d, J=3.0 Hz), 8.73 (1H, s), 9.92 (1H, br s).

### Example E-15

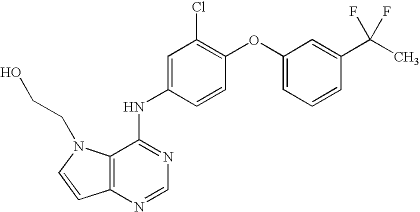

### Production of 2-[4-({3-chloro-4-[3-(1,1-difluoroethyl)phenoxy]phenyl}amino)-5H-pyrrolo[3,2-d]pyrimidin-5-yl]ethanol

### (i) Production of 1-[3-(2-chloro-4-nitrophenoxy)phenyl]ethanone

Using 2-chloro-1-fluoro-4-nitrobenzene (3.51 g), 3′-hydroxyacetophenone (2.72 g), potassium carbonate (4.15 g) and N,N-dimethylformamide (20 mL) and in the same manner as in Example E-7(i), the title compound (5.60 g) was obtained as a white solid.

¹H-NMR (CDCl₃) δ: 2.62 (3H, s), 6.92 (1H, d, J=9.1 Hz), 7.28-7.33 (1H, m), 7.56 (1H, t, J=8.0 Hz), 7.65 (1H, m), 7.82-7.88 (1H, m), 8.09 (1H, dd, J=2.6 Hz, 9.1 Hz), 8.41 (1H, d, J=2.6 Hz).

### (ii) Production of 2-chloro-1-[3-(1,1-difluoroethyl)phenoxy]-4-nitrobenzene

A solution of 1-[3-(2-chloro-4-nitrophenoxy)phenyl]ethanone (2.92 g), diethylaminosulfur trifluoride (8.06 g) in 1,2-dimethoxyethane (10 mL) was stirred at 50° C. for 5 days. The reaction mixture was poured into ice, and 8N aqueous sodium hydroxide solution (20 mL) was added thereto. Ethyl acetate was added, and the organic layer was separated, washed with saturated brine, and dried over anhydrous sodium sulfate. The solvent was evaporated under reduced pressure and the obtained residue was subjected to silica gel column chromatography (eluent, ethyl acetate:hexane=0:100→20:80). The objective fractions were concentrated under reduced pressure to give the title compound (2.75 g) as an orange solid.

¹H-NMR (CDCl₃) δ: 1.93 (3H, t, J=18.1 Hz), 6.92 (1H, d, J=9.1 Hz), 7.11-7.17 (1H, m), 7.25 (1H, m), 7.38-7.44 (1H, m), 7.51 (1H, t, J=7.8 Hz), 8.08 (1H, dd, J=2.7 Hz, 9.1 Hz), 8.40 (1H, d, J=2.7 Hz).

### (iii) Production of 3-chloro-4-[3-(1,1-difluoroethyl)phenoxy]aniline

Using 2-chloro-1-[3-(1,1-difluoroethyl)phenoxy]-4-nitrobenzene (2.51 g), reduced iron (1.99 g), calcium chloride (493 mg) and 10% water-containing ethanol (100 mL) and in the same manner as in Example E-13(i), the title compound (1.81 g) was obtained as a yellow oil.

¹H-NMR (CDCl₃) δ: 1.89(3H, t, J=18.1 Hz), 3.69 (2H, br s), 6.58 (1H, dd, J=2.7 Hz, 8.6 Hz), 6.79 (1H, d, J=2.7 Hz), 6.86-6.92 (1H, m), 6.91 (1H, d, J=8.6 Hz), 7.04 (1H, m), 7.13-7.18 (1H, m), 7.32 (1H, t, J=8.0 Hz).

### (iv) Production of 2-[4-({3-chloro-4-[3-(1,1-difluoroethyl)phenoxy]phenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (151 mg), 3-chloro-4-[3-(1,1-difluoroethyl)phenoxy]aniline (142 mg), isopropyl alcohol (3 mL), methanol (5 mL), tetrahydrofuran (1 mL) and 1N aqueous sodium hydroxide solution (1 mL) and in the same manner as in Example E-8, the title compound (149 mg) was obtained as a white powder.

¹H-NMR (CDCl₃) δ: 1.92 (3H, t, J=18.1 Hz), 4.10-4.18 (2H, m), 4.32-4.39 (2H, m), 6.06 (1H, d, J=3.2 Hz), 6.72 (1H, br s), 6.93 (1H, d, J=3.2 Hz), 6.97-7.02 (1H, m), 7.05 (1H, d, J=8.8 Hz), 7.13 (1H, m), 7.20 (1H, d, J=8.0 Hz), 7.36 (1H, t, J=7.8 Hz), 7.47 (1H, dd, J=2.5 Hz, 8.8 Hz), 7.81 (1H, d, J=2.5 Hz), 8.18 (1H, s), 9.61 (1H, br s).

### Example E-16

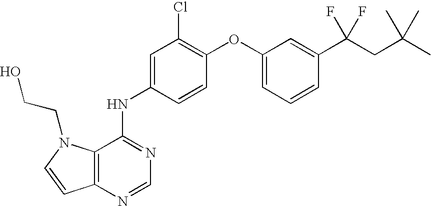

### Production of 2-[4-({3-chloro-4-[3-(1,1-difluoro-3,3-dimethylbutyl)phenoxy]phenyl}amino)-5H-pyrrolo[3,2-d]pyrimidin-5-yl]ethanol

### (i) Production of 1-[3-(2-chloro-4-nitrophenoxy)phenyl]-3,3-dimethylbutan-1-one

Using 2-chloro-1-fluoro-4-nitrobenzene (1.76 g), 1-(3-hydroxyphenyl)-3,3-dimethylbutan-1-one (1.92 g), potassium carbonate (2.07 g) and N,N-dimethylformamide (10 mL) and in the same manner as in Example E-7(i), the title compound (3.24 g) was obtained as a yellow oil.

¹H-NMR (CDCl₃) δ: 1.07 (9H, s), 2.85 (2H, s), 6.90 (1H, d, J=9.1 Hz), 7.25-7.30 (1H, m), 7.54 (1H, t, J=8.0 Hz), 7.63 (1H, m), 7.80-7.86 (1H, m), 8.08 (1H, dd, J=2.7 Hz, 9.1 Hz), 8.41 (1H, d, J=2.7 Hz).

### (ii) Production of 2-chloro-1-[3-(1,1-difluoro-3,3-dimethylbutyl)phenoxy]-4-nitrobenzene

Using 1-[3-(2-chloro-4-nitrophenoxy)phenyl]-3,3-dimethylbutan-1-one (1.90 g), diethylaminosulfur trifluoride (5.0 mL) and 1,2-dimethoxyethane (10 mL) and in the same manner as in Example E-15(ii), the title compound (52 mg) was obtained as a yellow oil.

¹H-NMR (CDCl₃) δ: 1.02 (9H, s), 2.07 (2H, t, J=19.1 Hz), 6.88 (1H, d, J=9.3 Hz), 7.09-7.15 (1H, m), 7.21 (1H, m), 7.38 (1H, d, J=7.7 Hz), 7.49 (1H, t, J=7.8 Hz), 8.08 (1H, dd, J=2.7 Hz, 9.3 Hz), 8.40 (1H, d, J=2.7 Hz).

### (iii) Production of 3-chloro-4-[3-(1,1-difluoro-3,3-dimethylbutyl)phenoxy]aniline

Using 2-chloro-1-[3-(1,1-difluoro-3,3-dimethylbutyl)phenoxy]-4-nitrobenzene (130 mg), reduced iron (99 mg), calcium chloride (25 mg) and 10% water-containing ethanol (10 mL) and in the same manner as in Example E-13(i), the title compound (100 mg) was obtained as a yellow oil.

¹H-NMR (CDCl₃) δ: 0.99 (9H, s), 2.04 (2H, t, J=18.9 Hz), 3.69 (2H, br s), 6.58 (1H, dd, J=2.7 Hz, 8.6 Hz), 6.79 (1H, d, J=2.7 Hz), 6.85-6.90 (1H, m), 6.90 (1H, d, J=8.6 Hz), 7.00 (1H, m), 7.12 (1H, d, J=7.7 Hz), 7.30 (1H, t, J=8.1 Hz).

### (iv) Production of 2-[4-({3-chloro-4-[3-(1,1-difluoro-3,3-dimethylbutyl)phenoxy]phenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (121 mg), 3-chloro-4-[3-(1,1-difluoro-3,3-dimethylbutyl)phenoxy]aniline (100 mg), isopropyl alcohol (5 mL), methanol (5 mL), tetrahydrofuran (1 mL) and 1N aqueous sodium hydroxide solution (1 mL) and in the same manner as in Example E-8, the title compound (67 mg) was obtained as a white powder.

¹H-NMR (CDCl₃) δ: 1.01 (9H, s), 2.07 (2H, t, =19.0 Hz), 4.11-4.18 (2H, m), 4.33-4.40 (2H, m), 6.09 (1H, d, J=3.3 Hz), 6.49 (1H, br s), 6.94 (1H, d, J=3.3 Hz), 6.95-7.01 (1H, m), 7.04 (1H, d, J=8.7 Hz), 7.10 (1H, m), 7.17 (1H, d, J=7.7 Hz), 7.35 (1H, t, J=8.0 Hz), 7.47 (1H, dd, J=2.6 Hz, 8.7 Hz), 7.81 (1H, d, J=2.6 Hz), 8.20 (1H, s), 9.55 (1H, br s).

### Example E-17

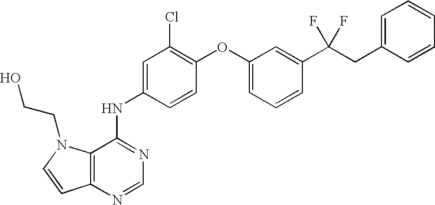

### Production of 2-[4-({3-chloro-4-[3-(1,1-difluoro-2-phenylethyl)phenoxy]phenyl}amino)-5H-pyrrolo[3,2-d]pyrimidin-5-yl]ethanol

### (i) Production of 1-[3-(2-chloro-4-nitrophenoxy)phenyl]-2-phenylethanone

Using 2-chloro-1-fluoro-4-nitrobenzene (702 mg), 1-(3-hydroxyphenyl)-2-phenylethanone (849 mg), potassium carbonate (829 mg) and N,N-dimethylformamide (10 mL) and in the same manner as in Example E-7(i), the title compound (725 mg) was obtained as a pale-yellow oil.

¹H-NMR (CDCl₃) δ: 4.26 (2H, s), 6.86 (1H, d, J=9.1 Hz), 7.20-7.36 (6H, m), 7.53 (1H, t, J=8.0 Hz), 7.65 (1H, m), 7.86-7.92 (1H, m), 8.05 (1H, dd, J=2.6 Hz, 9.1 Hz), 8.39 (1H, d, J=2.6 Hz).

### (ii) Production of 2-chloro-1-[3-(1,1-difluoro-2-phenylethyl)phenoxy]-4-nitrobenzene

Using 1-[3-(2-chloro-4-nitrophenoxy)phenyl]-2-phenylethanone (478 mg), diethylaminosulfur trifluoride (2.0 mL) and 1,2-dimethoxyethane (5 mL) and in the same manner as in Example E-15(ii), the title compound (372 mg) was obtained as a yellow oil.

¹H-NMR (CDCl₃) δ: 3.41 (2H, t, J=15.4 Hz), 6.59 (1H, d, J=9.1 Hz), 6.91 (1H, m), 7.01-7.13 (3H, m), 7.20-7.29 (4H, m), 7.44 (1H, t, J=8.0 Hz), 7.99 (1H, dd, J=2.5 Hz, 9.1 Hz), 8.35 (1H, d, J=2.5 Hz).

### (iii) Production of 3-chloro-4-[3-(1,1-difluoro-2-phenylethyl)phenoxy]aniline

Using 2-chloro-1-[3-(1,1-difluoro-2-phenylethyl)phenoxy]-4-nitrobenzene (372 mg), reduced iron (186 mg), calcium chloride (62 mg) and 10% water-containing ethanol (10 mL) and in the same manner as in Example E-13(i), the title compound (200 mg) was obtained as a yellow oil.

¹H-NMR (CDCl₃) δ: 3.36 (2H, t, J=15.9 Hz), 3.67 (2H, br s), 6.54 (1H, dd, J=2.7 Hz, 8.8 Hz), 6.77 (1H, d, J=2.7 Hz), 6.78-6.84 (2H, m), 6.85-6.91 (1H, m), 6.98 (1H, d, J=8.0 Hz), 7.05-7.12 (2H, m), 7.20-7.30 (4H, m).

### (iv) Production of 2-[4-({3-chloro-4-[3-(1,1-difluoro-2-phenylethyl)phenoxy]phenyl}amino)-5H-pyrrolo[3,2-d]pyrimidin-5-yl]ethanol

Using 2-(4-chloro-5H-pyrrolo[3,2-d]pyrimidin-5-yl)ethyl benzoate (169 mg), 3-chloro-4-[3-(1,1-difluoro-2-phenylethyl)phenoxy]aniline (200 mg), isopropyl alcohol (3 mL), methanol (5 mL), tetrahydrofuran (1 mL) and 1N aqueous sodium hydroxide solution (1 mL) and in the same manner as in Example E-8, the title compound (171 mg) was obtained as a white powder.

¹H-NMR (CDCl₃) δ: 3.39 (2H, t, J=15.8 Hz), 4.10-4.19 (2H, m), 4.32-4.40 (2H, m), 6.09 (1H, d, J=3.3 Hz), 6.53 (1H, br s), 6.90-7.14 (7H, m), 7.20-7.38 (4H, m), 7.45 (1H, dd, J=2.5 Hz, 8.8 Hz), 7.80 (1H, d, J=2.5 Hz), 8.21 (1H, s), 9.57 (1H, br s).

### Example E-18

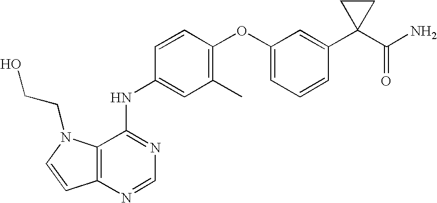

### Production of 1-[3-(4-{[5-(2-hydroxyethyl)-5H-pyrrolo[3,2-d]pyrimidin-4-yl]amino}-2-methylphenoxy)phenyl]cyclopropanecarboxamide

### (i) Production of 1-(3-methoxyphenyl)cyclopropanecarbonitrile

60% Sodium hydride (2.72 g) was suspended in N,N-dimethylformamide (80 mL) and the mixture was cooled to 0° C. A solution of (3-methoxyphenyl)acetonitrile (4.00 g) in N,N-dimethylformamide (20 mL) was added dropwise to the mixture and the mixture was stirred at 0° C. for 1 hr. 1,2-Dibromoethane (3.5 mL) was added dropwise to the reaction mixture, and the mixture was stirred at room temperature for 17 hr. A saturated aqueous ammonium chloride solution was added to the reaction mixture, and the mixture was extracted with diethyl ether. The organic layer was washed successively with water and saturated brine, dried over anhydrous magnesium sulfate and concentrated under reduced pressure. The residue was separated and purified by silica gel column chromatography (hexane:ethyl acetate=100:0→80:20) to give the title compound (2.76 g) as a yellow oil.

¹H-NMR (CDCl₃) δ: 1.36-1.44 (2H, m), 1.67-1.75 (2H, m), 3.81 (3H, s), 6.77-6.89 (3H, m), 7.21-7.29 (1H, m).

### (ii) Production of 1-(3-methoxyphenyl)cyclopropanecarboxamide and 1-(3-methoxyphenyl)cyclopropanecarboxylic acid

1-(3-Methoxyphenyl)cyclopropanecarbonitrile (2.75 g) was dissolved in a mixed solvent of ethanol (100 mL)/water (50 mL), potassium hydroxide (8.94 g) was added thereto, and the mixture was stirred with heating under reflux for 5 hr. The reaction mixture was concentrated under reduced pressure, the residue was adjusted to pH 1-2 with 6N hydrochloric acid, and the mixture was extracted with ethyl acetate. The organic layer was washed successively with water and saturated brine, dried over anhydrous magnesium sulfate and concentrated under reduced pressure. The residue was separated and purified by silica gel column chromatography (hexane:ethyl acetate=50:50→0:100) to give 1-(3-methoxyphenyl)cyclopropanecarboxamide (762 mg) and 1-(3-methoxyphenyl)cyclopropanecarboxylic acid (1.78 g), each as pale-orange crystals.

### 1-(3-methoxyphenyl)cyclopropanecarboxamide

¹H-NMR (DMSO-d₆) δ: 0.91-0.98 (2H, m), 1.26-1.34 (2H, m), 3.75 (3H, s), 6.11 (1H, br s), 6.79-6.96 (3H, m), 7.02 (1H, br s), 7.25 (1H, t, J=8.0 Hz).

### 1-(3-methoxyphenyl)cyclopropanecarboxylic acid

¹H-NMR (DMSO-d₆) δ: 1.02-1.20 (2H, m), 1.33-1.50 (2H, m), 3.73 (3H, s), 6.71-7.00 (3H, m), 7.20 (1H, t, J=7.8 Hz), 12.27 (1H, br s).

### (iii) Production of 1-(3-hydroxyphenyl)cyclopropanecarboxamide

1-(3-Methoxyphenyl)cyclopropanecarboxamide (756 mg) was dissolved in benzotrifluoride (20 mL), 1N boron tribromide/dichloromethane solution (10 mL) was added thereto at 0° C., and the mixture was stirred at room temperature for 2 days. The reaction mixture was ice-cooled, saturated aqueous sodium hydrogencarbonate solution was added, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=67:33→0:100) to give the title compound (350 mg) as a white powder.

¹H-NMR (DMSO-d₆) δ: 0.85-0.95 (2H, m), 1.22-1.32 (2H, m), 6.04 (1H, br s), 6.59-6.71 (1H, m), 6.71-6.82 (2H, m), 7.02 (1H, br s), 7.12 (1H, t, J=7.8 Hz), 9.40 (1H, s).

### (iv) Production of 1-[3-(2-methyl-4-nitrophenoxy)phenyl]cyclopropanecarboxamide

A mixture of 1-(3-hydroxyphenyl)cyclopropanecarboxamide (345 mg) and 2-fluoro-5-nitrotoluene (347 mg) was dissolved in N,N-dimethylformamide (5 mL), potassium carbonate (588 mg) was added thereto, and the mixture was stirred at room temperature for 19 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=67:33→10:90) to give the title compound (561 mg) as a white powder.

¹H-NMR (CDCl₃) δ: 1.11 (2H, q, J=3.9 Hz), 1.58-1.70 (2H, m), 2.41 (3H, s), 5.35 (1H, br s), 5.56 (1H, br s), 6.80 (1H, d, J=9.0 Hz), 6.93-7.00 (1H, m), 7.13 (1H, t, J=2.1 Hz), 7.27-7.33 (1H, m), 7.41 (1H, t, J=7.8 Hz), 8.02 (1H, dd, J=2.0 Hz, 9.0 Hz), 8.18 (1H, d, J=2.0 Hz).

### (v) Production of 1-[3-(4-amino-2-methylphenoxy)phenyl]cyclopropanecarboxamide

1-[3-(2-Methyl-4-nitrophenoxy)phenyl]cyclopropanecarboxamide (556 mg) was suspended in a mixed solvent of ethanol (18 mL)/water (2 mL), reduced iron (615 mg) and calcium chloride (105 mg) were added thereto, and the mixture was stirred with heating under reflux for 4 hr. The reaction mixture was filtered through celite, and the filtrate was concentrated under reduced pressure. The residue was dissolved in ethyl acetate, and the solution was washed successively with water and saturated brine, dried over anhydrous magnesium sulfate and concentrated under reduced pressure. The residue was separated and purified by silica gel column chromatography (hexane:ethyl acetate=50:50→0:100) to give the title compound (433 mg) as a brown oil.

¹H-NMR (CDCl₃) δ: 1.03-1.11 (2H, m), 1.54-1.63 (2H, m), 2.09 (3H, s), 3.59 (2H, br s), 5.38 (2H, br s), 6.49-6.56 (1H, m), 6.60 (1H, d, J=2.8 Hz), 6.72-6.82 (2H, m), 6.89-6.94 (1H, m), 7.01-7.07 (1H, m), 7.23 (1H, t, J=8.4 Hz).

### (vi) Production of 1-[3-(4-{[5-(2-hydroxyethyl)-5H-pyrrolo[3,2-d]pyrimidin-4-yl]amino}-2-methylphenoxy)phenyl]cyclopropanecarboxamide

A mixture of 2-(4-chloro-5H-pyrrolo[3,2-d]pyrimidin-5-yl)ethyl benzoate (113 mg) and 1-[3-(4-amino-2-methylphenoxy)phenyl]cyclopropanecarboxamide (108 mg) was dissolved in isopropyl alcohol (3 mL), and the mixture was stirred at 70° C. for 21 hr. The reaction mixture was cooled to room temperature, 1N aqueous sodium hydroxide solution (1 mL) was added thereto, and the mixture was stirred at room, temperature for 1 hr. A saturated aqueous ammonium chloride solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=20:80→0:100→ethyl acetate:methanol=90:10) and crystallized from ethyl acetate to give the title compound (110 mg) as white crystals.

¹H-NMR (DMSO-d₆) δ: 0.0.90-0.98 (2H, m), 1.25-1.33 (2H, m), 2.16 (3H, s), 3.87 (2H, t, J=4.5 Hz), 4.52 (2H, t, J=4.5 Hz), 6.25 (2H, br s), 6.48 (1H, d, J=3.0 Hz), 6.74-6.82 (1H, m), 6.82-6.87 (1H, m), 6.96-7.09 (3H, m), 7.30 (1H, t, J=7.9 Hz), 7.47-7.56 (2H, m), 7.62 (1H, d, J=3.0 Hz), 8.27 (1H, s), 9.61 (1H, br s).

### Example E-19

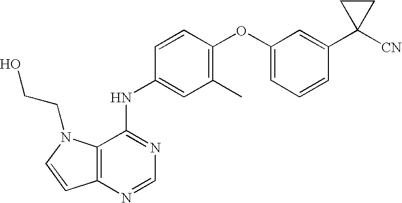

### Production of 1-[3-(4-{[5-(2-hydroxyethyl)-5H-pyrrolo[3,2-d]pyrimidin-4-yl]amino}-2-methylphenoxy)phenyl]cyclopropanecarbonitrile

### (i) Production of 1-(3-hydroxyphenyl)cyclopropanecarbonitrile

Using 1-(3-methoxyphenyl)cyclopropanecarbonitrile (1.28 g), toluene (75 mL) and 1N boron tribromide/dichloromethane solution (25 mL) and in the same manner as in Example E-18(iii), the title compound (1.11 g) was obtained as a colorless oil.

¹H-NMR (CDCl₃) δ: 1.37-1.46 (2H, m), 1.69-1.77 (2H, m), 5.16 (1H, s), 6.70-6.81 (2H, m), 6.83 (1H, t, J=2.1 Hz), 7.21 (1H, t, J=7.8 Hz).

### (ii) Production of 1-[3-(2-methyl-4-nitrophenoxy)phenyl]cyclopropanecarbonitrile

Using 2-fluoro-5-nitrotoluene (409 mg), 1-(3-hydroxyphenyl)cyclopropanecarbonitrile (450 mg), potassium carbonate (731 mg) and N,N-dimethylformamide (8 mL) and in the same manner as in Example E-18(iv), the title compound (776 mg) was obtained as a white powder.

¹H-NMR (CDCl₃) δ: 1.38-1.47 (2H, m), 1.72-1.82 (2H, m), 2.40 (3H, s), 6.78 (1H, d, J=9.0 Hz), 6.88-6.95 (1H, m), 7.01 (1H, t, J=2.1 Hz), 7.10-7.17 (1H, m), 7.38 (1H, t, J=8.0 Hz), 8.01 (1H, dd, J=2.8 Hz, 9.0 Hz), 8.12-8.22 (1H, m).

### (iii) Production of 1-[3-(4-amino-2-methylphenoxy)phenyl]cyclopropanecarbonitrile

Using 1-[3-(2-methyl-4-nitrophenoxy)phenyl]cyclopropanecarbonitrile (771 mg), reduced iron (847 mg), calcium chloride (151 mg) and ethanol (27 mL)/water (3 mL) and in the same manner as in Example E-18(v), the title compound (665 mg) was obtained as a yellow oil.

¹H-NMR (CDCl₃) δ: 1.36-1.39 (2H, m), 1.67-1.71 (2H, m), 2.09 (3H, s), 3.57 (2H, br s), 6.52 (1H, dd, J=2.7 Hz, 8.7 Hz), 6.59 (1H, d, J=2.7 Hz), 6.67-6.70 (1H, m), 6.75-6.79 (2H, m), 6.93-6.96 (1H, m), 7.21 (1H, t, J=8.1 Hz).

### (iv) Production of 1-[3-(4-{[5-(2-hydroxyethyl)-5H-pyrrolo[3,2-d]pyrimidin-4-yl]amino}-2-methylphenoxy)phenyl]cyclopropanecarbonitrile

Using 2-(4-chloro-5H-pyrrolo[3,2-d]pyrimidin-5-yl)ethyl benzoate (127 mg), 1-[3-(4-amino-2-methylphenoxy)phenyl]cyclopropanecarbonitrile (130 mg), isopropyl alcohol (3 mL) and 1N aqueous sodium hydroxide solution (1 mL) and in the same manner as in Example E-18(vi), the title compound (115 mg) was obtained as a white powder.

¹H-NMR (CDCl₃) δ: 1.38-1.45 (2H, m), 1.69-1.76 (2H, m), 2.24 (3H, s), 4.08-4.18 (2H, m), 4.31-4.43 (2H, m), 6.12 (1H, d, J=3.3 Hz), 6.35 (1H, br s), 6.80 (1H, dd, J=1.9 Hz, 8.0 Hz), 6.88-7.03 (4H, m), 7.25 (1H, t, J=8.0 Hz), 7.38-7.51 (2H, m), 8.22 (1H, s), 9.31 (1H, m).

### Example E-20

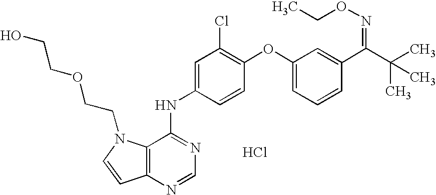

### Production of (1Z)-1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one O-ethyloxime hydrochloride

Using 1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one (255 mg), ethanol (5 mL), O-ethylhydroxylamine hydrochloride (146 mg), sodium acetate (123 mg) and 1N hydrogen chloride/ethyl acetate solution (0.5 mL) and in the same manner as in Example E-9(iii), the title compound (144 mg) was obtained as a white powder.

¹H-NMR (DMSO-d₆) δ: 1.07 (3H, t, J=7.0 Hz), 1.10 (9H, s), 3.40-3.50 (4H, m), 3.84 (2H, t, J=4.5 Hz), 3.92 (2H, q, J=7.0 Hz), 4.82 (2H, t, J=4.5 Hz), 6.61 (1H, m), 6.69 (1H, d, J=3.0 Hz), 6.82 (1H, d, J=7.4 Hz), 6.93-6.99 (1H, m), 7.19 (1H, d, J=8.8 Hz), 7.42 (1H, t, J=7.8 Hz), 7.61 (1H, dd, J=2.5 Hz, 8.8 Hz), 7.95 (1H, d, J=2.5 Hz), 8.01 (1H, d, J=3.0 Hz), 8.71 (1H, s), 9.92 (1H, br s).

### Example E-21

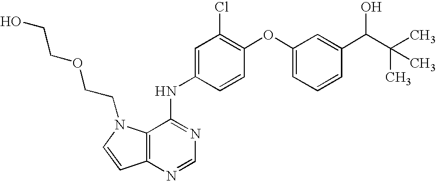

### Production of 1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-ol

To a solution of 1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one (255 mg) in methanol (5 mL) was added sodium borohydride (38 mg) and the mixture was stirred at room temperature for 4 hr. Water was added to the reaction mixture, and the mixture was extracted with ethyl acetate. The organic layer was washed with saturated brine and dried over anhydrous magnesium sulfate. The solvent was evaporated under reduced pressure and the obtained residue was crystallized from ethyl acetate-diisopropyl ether to give the title compound (206 mg) as a white powder.

¹H-NMR (CDCl₃) δ: 0.92 (9H, s), 2.02 (1H, br s), 2.12 (1H, br s), 3.69-3.80 (4H, m), 4.01 (2H, t, J=4.4 Hz), 4.36 (1H, s), 4.55 (2H, t, J=4.4 Hz), 6.59 (1H, d, J=3.0 Hz), 6.82-6.88 (1H, m), 6.92 (1H, m), 6.96-7.04 (2H, m), 7.19 (1H, d, J=3.0 Hz), 7.24 (1H, t, J=7.8 Hz), 7.54 (1H, dd, J=2.5 Hz, 8.8 Hz), 7.85 (1H, d, J=2.5 Hz), 8.47 (1H, s), 8.73 (1H, br s).

### Example E-22

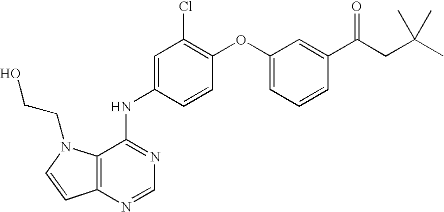

### Production of 1-[3-(2-chloro-4-{[5-(2-hydroxyethyl)-5H-pyrrolo[3,2-d]pyrimidin-4-yl]amino}phenoxy)phenyl]-3,3-dimethylbutan-1-one

### (i) Production of 1-[3-(4-amino-2-chlorophenoxy)phenyl]-3,3-dimethylbutan-1-one

To a solution of 1-[3-(2-chloro-4-nitrophenoxy)phenyl]-3,3-dimethylbutan-1-one (1.04 g) in ethyl acetate (20 mL) was added 5% platinum-activated carbon (50 mg) and the mixture was stirred under a hydrogen atmosphere at room temperature for 3 hr. The catalyst was filtered off, and the filtrate was concentrated. The obtained residue was subjected to silica gel column chromatography (eluent, ethyl acetate:hexane=15:85→35:65) and the objective fractions were concentrated under reduced pressure to give the title compound (964 mg) as a brown oil.

¹H-NMR (CDCl₃) δ: 1.04 (9H, s), 2.80 (2H, s), 3.69 (2H, br s), 6.57 (1H, dd, J=2.7 Hz, 8.7 Hz), 6.78 (1H, d, J=2.7 Hz), 6.90 (1H, d, J=8.7 Hz), 7.05 (1H, ddd, J=0.9 Hz, 2.6 Hz, 8.1 Hz), 7.34 (1H, t, J=8.0 Hz), 7.41 (1H, m), 7.55-7.59 (1H, m).

### (ii) Production of 1-[3-(2-chloro-4-{[5-(2-hydroxyethyl)-5H-pyrrolo[3,2-d]pyrimidin-4-yl]amino}phenoxy)phenyl]-3,3-dimethylbutan-1-one

Using 2-(4-chloro-5H-pyrrolo[3,2-d]pyrimidin-5-yl)ethyl benzoate (754 mg), 1-[3-(4-amino-2-chlorophenoxy)phenyl]-3,3-dimethylbutan-1-one (795 mg), isopropyl alcohol (5 mL), methanol (10 mL), tetrahydrofuran (2 mL) and 1N aqueous sodium hydroxide solution (2.5 mL) and in the same manner as in Example E-8, the title compound (878 mg) was obtained as a white powder.

¹H-NMR (CDCl₃) δ: 1.06 (9H, s), 2.84 (2H, s), 4.13 (2H, t, J=4.4 Hz), 4.37 (2H, t, J=4.4 Hz), 6.09 (1H, d, J=3.2 Hz), 6.58 (1H, br s), 6.95 (1H, d, J=3.2 Hz), 7.04 (1H, d, J=8.8 Hz), 7.13-7.18 (1H, m), 7.39 (1H, t, J=8.0 Hz), 7.46 (1H, dd, J=2.6 Hz, 8.8 Hz), 7.50 (1H, m), 7.62 (1H, d, J=7.7 Hz), 7.81 (1H, d, J=2.6 Hz), 8.19 (1H, s), 9.59 (1H, br s).

### Example E-23

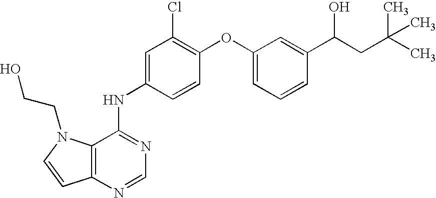

### Production of 1-[3-(2-chloro-4-{[5-(2-hydroxyethyl)-5H-pyrrolo[3,2-d]pyrimidin-4-yl]amino}phenoxy)phenyl]-3,3-dimethylbutan-1-ol

Using 1-[3-(2-chloro-4-{[5-(2-hydroxyethyl)-5H-pyrrolo[3,2-d]pyrimidin-4-yl]amino}phenoxy)phenyl]-3,3-dimethylbutan-1-one (239 mg), methanol (5 mL) and sodium borohydride (38 mg) and in the same manner as in Example E-21, the title compound (234 mg) was obtained as a white powder.

¹H-NMR (CDCl₃) δ: 1.00 (9H, s), 1.61 (1H, dd, J=3.4 Hz, 14.5 Hz), 1.74 (1H, dd, J=8.3 Hz, 14.5 Hz), 1.91 (1H, br s), 4.13 (2H, t, J=4.5 Hz), 4.36 (2H, t, J=4.5 Hz), 4.82 (1H, dd, J=3.4 Hz, 8.3 Hz), 6.09 (1H, d, J=3.0 Hz), 6.52 (1H, br s), 6.85 (1H, dd, J=2.5 Hz, 8.0 Hz), 6.95 (1H, d, J=3.0 Hz), 7.00-7.08 (3H, m), 7.28 (1H, t, J=7.8 Hz), 7.45 (1H, dd, J=2.6 Hz, 8.8 Hz), 7.80 (1H, d, J=2.6 Hz), 8.19 (1H, s), 9.56 (1H, br s).

### Example E-24

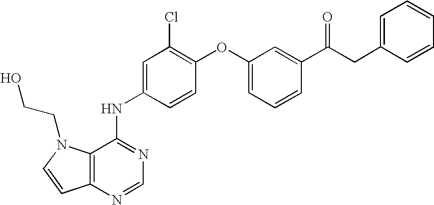

### Production of 1-[3-(2-chloro-4-{[5-(2-hydroxyethyl)-5H-pyrrolo[3,2-d]pyrimidin-4-yl]amino}phenoxy)phenyl]-2-phenylethanone

### (i) Production of 1-[3-(4-amino-2-chlorophenoxy)phenyl]-2-phenylethanone

Using 1-[3-(2-chloro-4-nitrophenoxy)phenyl]-2-phenylethanone (240 mg), ethyl acetate (10 mL) and 5% platinum-activated carbon (12 mg) and in the same manner as in Example E-22(i), the title compound (192 mg) was obtained as a colorless oil.

¹H-NMR (CDCl₃) δ: 3.70 (2H, br s), 4.21 (2H, s), 6.57 (1H, dd, J=2.7 Hz, 8.8 Hz), 6.78 (1H, d, J=2.7 Hz), 6.89 (1H, d, J=8.8 Hz), 7.06-7.11 (1H, m), 7.19-7.40 (6H, m), 7.46 (1H, m), 7.64-7.69 (1H, m).

### (ii) Production of 1-[3-(2-chloro-4-{[5-(2-hydroxyethyl)-5H-pyrrolo[3,2-d]pyrimidin-4-yl]amino}phenoxy)phenyl]-2-phenylethanone

Using 2-(4-chloro-5H-pyrrolo[3,2-d]pyrimidin-5-yl)ethyl benzoate (166 mg), 1-[3-(4-amino-2-chlorophenoxy)phenyl]-2-phenylethanone (186 mg), isopropyl alcohol (3 mL), methanol (5 mL), tetrahydrofuran (1 mL) and 1N aqueous sodium hydroxide solution (1 mL) and in the same manner as in Example E-8, the title compound (123 mg) was obtained as a pale-brown powder.

¹H-NMR (CDCl₃) δ: 4.14 (2H, t, J=4.5 Hz), 4.25 (2H, s), 4.38 (2H, t, J=4.5 Hz), 6.15 (1H, d, J=3.2 Hz), 6.19 (1H, br s), 6.97 (1H, d, J=3.2 Hz), 7.05 (1H, d, J=8.7 Hz), 7.17-7.37 (6H, m), 7.42 (1H, t, J=8.0 Hz), 7.49 (1H, dd, J=2.7 Hz, 8.7 Hz), 7.54 (1H, m), 7.72 (1H, d, J=7.7 Hz), 7.82 (1H, d, J=2.7 Hz), 8.24 (1H, s), 9.55 (1H, br s).

### Example E-25

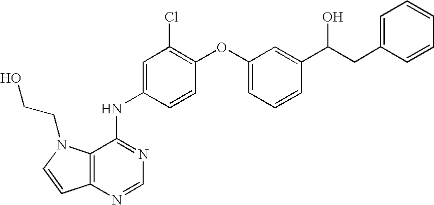

### Production of 1-[3-(2-chloro-4-{[5-(2-hydroxyethyl)-5H-pyrrolo[3,2-d]pyrimidin-4-yl]amino}phenoxy)phenyl]-2-phenylethanol

Using 1-[3-(2-chloro-4-{[5-(2-hydroxyethyl)-5H-pyrrolo[3,2-d]pyrimidin-4-yl]amino}phenoxy)phenyl]-2-phenylethanone (60 mg), methanol (2 mL) and sodium borohydride (10 mg) and in the same manner as in Example E-21, the title compound (24 mg) was obtained as a white powder.

¹H-NMR (CDCl₃) δ: 2.08 (1H, br s), 2.98 (1H, dd, J=8.2 Hz, 13.7 Hz), 3.05 (1H, dd, J=4.,9 Hz, 13.7 Hz), 4.14 (2H, t, J=4.6 Hz), 4.38 (2H, t, J=4.6 Hz), 4.89 (1H, dd, J=4.9 Hz, 8.2 Hz), 6.07 (1H, br s), 6.15 (1H, d, J=3.3 Hz), 6.87-6.93 (1H, m), 6.96-7.02 (3H, m), 7.08 (1H, d, J=7.7 Hz), 7.17-7.35 (6H, m), 7.45 (1H, dd, J=2.5 Hz, 8.8 Hz), 7.79 (1H, d, J=2.5 Hz), 8.24 (1H, s), 9.48 (1H, br s).

### Example E-26

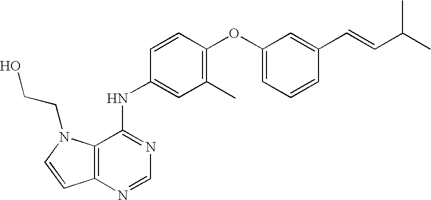

### Production of 2-{4-[(3-methyl-4-{3-[(1E)-3-methylbut-1-en-1-yl]phenoxy}phenyl)amino]-5H-pyrrolo[3,2-d]pyrimidin-5-yl}ethanol

### (i) Production of 3-(2-methyl-4-nitrophenoxy)benzaldehyde

Using 2-fluoro-5-nitrotoluene (3.00 g), 3-hydroxybenzaldehyde (2.59 g), potassium carbonate (4.58 g) and N,N-dimethylformamide (30 mL) and in the same manner as in Example E-18(iv), the title compound (4.14 g) was obtained was obtained as a pale-yellow solid.

¹H-NMR (CDCl₃) δ: 2.40 (3H, s), 6.85 (1H, d, J=9 Hz), 7.28-7.36 (1H, m), 7.46-7.54 (1H, m), 7.60 (1H, t, J=7.8 Hz), 7.68-7.76 (1H, m), 8.04 (1H, dd, J=2.8 Hz, 9.0 Hz), 8.19 (1H, d, J=2.8 Hz), 10.00 (1H, s).

### (ii) Production of 2-methyl-1-{3-[3-methylbut-1-en-1-yl]phenoxy}-4-nitrobenzene

Isobutyltriphenylphosphonium bromide (1.86 g) was suspended in tetrahydrofuran (20 mL) and the suspension was cooled to 0° C. 1.6 M n-butyllithium/hexane solution (3.5 mL) was added dropwise, and the mixture was stirred at 0° C. for 1 hr. A solution of 3-(2-methyl-4-nitrophenoxy)benzaldehyde (1.00 g) in tetrahydrofuran (10 mL) was added dropwise to the reaction mixture, and the mixture was stirred at room temperature for 2 hr. A saturated aqueous ammonium chloride solution was added to the reaction mixture, and the mixture was extracted with diethyl ether. The organic layer was washed with saturated brine, dried over anhydrous magnesium sulfate and concentrated under reduced pressure. The residue was separated and purified by silica gel column chromatography (hexane:ethyl acetate=100:0→90:10) to give the title compound (1.09 g, E/Z=about 1/5) as a yellow oil.

¹H-NMR (CDCl₃) δ: 1.03 (5H, d, J=6.5 Hz), 1.09 (1H, d, J=6.5 Hz), 2.41 (3H, s), 2.40-2.51 (0.17H, m), 2.77-2.92 (0.83H, m), 5.51 (0.83H, dd, J=10.3 Hz, 11.6 Hz), 6.15-6.38 (1.17H, m), 6.72-6.96 (2.83H, m), 7.03 (0.17H, t, J=7.7 Hz), 7.11 (0.83H, d, J=7.7 Hz), 7.20 (0.17H, d, J=7.7 Hz), 7.28-7.41 (1H, m), 7.94-8.06 (1H, m), 8.11-8.21 (1H, m).

### (iii) Production of 3-methyl-4-{3-[3-methylbut-1-en-1-yl]phenoxy}aniline

Using 2-methyl-1-{3-[3-methylbut-1-en-1-yl]phenoxy}-4-nitrobenzene (1.08 g), reduced iron (1.01 g), calcium chloride (205 mg) and ethanol (36 mL)/water (4 mL) and in the same manner as in Example E-18(v), the title compound (821 mg, E/Z=about 1/5) was obtained as a yellow oil.

¹H-NMR (CDCl₃) δ: 1.00 (5H, d, J=6.6 Hz), 1.07 (1H, d, J=6.6 Hz), 2.12 (3H, s), 2.36-2.52 (0.17H, m), 2.74-2.96 (0.83H, m), 3.55 (2H, br s), 5.43 (0.83H, dd, J=10.2 Hz, 11.6 Hz), 6.08-6.33 (1.17H, m), 6.47-6.92 (5.83H, m), 6.95-7.01 (0.17H, m), 7.16 (0.17H, t, J=8.0 Hz), 7.19 (0.83H, t, J=8.0 Hz).

### (iv) Production of 3-methyl-4-{3-[(1E)-3-methylbut-1-en-1-yl]phenoxy}aniline

3-Methyl-4-{3-[3-methylbut-1-en-1-yl]phenoxy}aniline (815 mg) was dissolved in tetrahydrofuran (15 mL), diphenyl disulfide (133 mg) and 2,2′-azobis(butyronitrile) (103 mg) were added, and the mixture was stirred with heating under reflux for 3 days. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=90:10→50:50) to give the title compound (181 mg) as an orange oil.

¹H-NMR (CDCl₃) δ: 1.06 (6H, d, J=6.9 Hz), 2.11 (3H, s), 2.35-2.53 (1H, m), 3.55 (2H, br s), 6.13 (1H, dd, J=6.3 Hz, 16.0 Hz), 6.25 (1H, d, J=16.0 Hz), 6.51 (1H, dd, J=3.0 Hz, 8.4 Hz), 6.59 (1H, d, J=3.0 Hz), 6.65 (1H, dd, J=2.6 Hz, 8.0 Hz), 6.78 (1H, d, J=8.4 Hz), 6.81-6.86 (1H, m), 6.97 (1H, d, J=8.0 Hz), 7.15 (1H, t, J=8.0 Hz).

### (v) Production of 2-{4-[(3-methyl-4-{3-[(1E)-3-methylbut-1-en-1-yl]phenoxy}phenyl)amino]-5H-pyrrolo[3,2-d]pyrimidin-5-yl}ethanol

Using 2-(4-chloro-5H-pyrrolo[3,2-d]pyrimidin-5-yl)ethyl benzoate (69.6 mg), 3-methyl-4-{3-[(1E)-3-methylbut-1-en-1-yl]phenoxy}aniline (65.0 mg), isopropyl alcohol (2 mL) and 1N aqueous sodium hydroxide solution (1 mL) and in the same manner as in Example E-18(vi), the title compound (47.7 mg) was obtained was obtained as a white powder.

¹H-NMR (CDCl₃) δ: 1.09 (6H, d, J=6.8 Hz), 2.26 (3H, s), 2.36-2.56 (1H, m), 4.13 (2H, t, J=4.5 Hz), 4.31-4.42 (2H, m), 6.12 (1H, d, J=3.2 Hz), 6.19 (1H, dd, J=Hz, 16.2 Hz), 6.30 (1H, d, J=16.2 Hz), 6.72-6.81 (1H, m), 6.90-6.99 (3H, m), 7.04 (1H, d, J=8.0 Hz), 7.21 (1H, t, J=8.0 Hz), 7.37-7.48 (2H, m), 8.23 (1H, s), 9.28 (1H, s).

### Example E-27

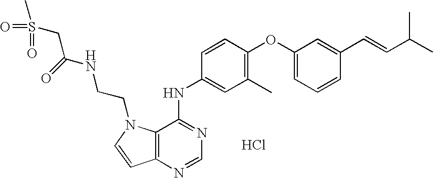

### Production of N-(2-{4-[(3-methyl-4-{3-[(1E)-3-methylbut-1-en-1-yl]phenoxy}phenyl)amino]-5H-pyrrolo[3,2-d]pyrimidin-5-yl}ethyl)-2-(methylsulfonyl)acetamide hydrochloride

### (i) Production of tert-butyl(2-{4-[(3-methyl-4-{3-[(1E)-3-methylbut-1-en-1-yl]phenoxy}phenyl)amino]-5H-pyrrolo[3,2-d]pyrimidin-5-yl}ethyl)carbamate

A mixture of tert-butyl[2-(4-chloro-5H-pyrrolo[3,2-d]pyrimidin-5-yl)ethyl]carbamate (120 mg) and 3-methyl-4-{3-[(1E)-3-methylbut-1-en-1-yl]phenoxy}aniline (109 mg) was dissolved in isopropyl alcohol (5 mL), and stirred at 70° C. for 22 hr. A saturated aqueous sodium hydrogencarbonate solution was added to the reaction mixture, and the mixture was extracted with ethyl acetate. The organic layer was washed successively with water and saturated brine, dried over anhydrous magnesium sulfate and concentrated under reduced pressure. The residue was separated and purified by silica gel column chromatography (hexane:ethyl acetate=67:33→10:90) to give the title compound (186 mg) as a white powder.

¹H-NMR (CDCl₃) δ: 1.08 (6H, d, J=6.6 Hz), 1.46 (9H, s), 2.24 (3H, s), 2.35-2.55 (1H, m), 3.42-3.58 (2H, m), 4.40-4.52 (2H, m), 4.95-5.08 (1H, m), 6.18 (1H, dd, J=6.6 Hz, 16.2 Hz), 6.29 (1H, d, J=16.2 Hz), 6.58 (1H, d, J=3.0 Hz), 6.73-6.81 (1H, m), 6.90-6.98 (2H, m), 7.03 (1H, d, J=8.0 Hz), 7.16 (1H, d, J=3.0 Hz), 7.20 (1H, t, J=8.0 Hz), 7.58-7.70 (2H, m), 8.32 (1H, br s), 8.49 (1H, s).

### (ii) Production of 5-(2-aminoethyl)-N₇(3-methyl-4-{3-[(1E)-3-methylbut-1-en-1-yl]phenoxy}phenyl)-5H-pyrrolo[3,2-d]pyrimidin-4-amine dihydrochloride

tert-Butyl(2-{4-[(3-methyl-4-{3-[(1E)-3-methylbut-1-en-1-yl]phenoxy}phenyl)amino]-5H-pyrrolo[3,2-d]pyrimidin-5-yl}ethyl)carbamate (180 mg) was dissolved in ethanol (1 mL), 6N hydrochloric acid (0.3 mL) was added thereto, and the mixture was stirred at 50° C. for 12 hr. The reaction mixture was concentrated under reduced pressure, the residue was dissolved in ethanol, and the mixture was concentrated again under reduced pressure. The precipitate was collected by filtration to give the title compound (158 mg) as a pale-yellow powder.

¹H-NMR (DMSO-d₆) δ: 1.05 (6H, d, J=6.8 Hz), 2.22 (3H, s), 2.37-2.54 (1H, m), 3.18-3.32 (2H, m), 5.00 (2H, t, J=6.1 Hz), 6.22-6.41 (2H, m), 6.67-6.79 (2H, m), 6.92-7.04 (2H, m), 7.16 (1H, d, J=7.9 Hz), 7.31 (1H, t, J=7.9 Hz), 7.39 (1H, dd, J=2.0 Hz, 8.6 Hz), 7.49 (1H, d, J=2.0 Hz), 8.03 (1H, d, J=3.2 Hz), 8.31 (3H br s), 8.67 (1H, s), 9.87 (1H, br s).

### (iii) Production of N-(2-{4-[(3-methyl-4-{3-[(1E)-3-methylbut-1-en-1-yl]phenoxy}phenyl)amino]-5H-pyrrolo[3,2-d]pyrimidin-5-yl}ethyl)-2-(methylsulfonyl)acetamide hydrochloride

A mixture of 5-(2-aminoethyl)-N-(3-methyl-4-{3-[(1E)-3-methylbut-1-en-1-yl]phenoxy}phenyl)-5H-pyrrolo[3,2-d]pyrimidin-4-amine dihydrochloride (74.8 mg) and methylsulfonylacetic acid (31.1 mg) was dissolved in tetrahydrofuran (0.4 mL)/N,N-dimethylformamide (0.4 mL), 1-hydroxybenzotriazole (32.5 mg), triethylamine (0.2 mL) and 1-ethyl-3-(3-dimethylaminopropyl)carbodiimide hydrochloride (45.0 mg) were added successively thereto, and the mixture was stirred at room temperature for 24 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=20:80→0:100→ethyl acetate:methanol=90:10) to give N-(2-{4-[(3-methyl-4-{3-[(1E)-3-methylbut-1-en-1-yl]phenoxy}phenyl)amino]-5H-pyrrolo[3,2-d]pyrimidin-5-yl}ethyl)-2-(methylsulfonyl)acetamide. The compound was dissolved in ethyl acetate and treated with 4N hydrogen chloride/ethyl acetate solution, and the precipitate was collected by filtration to give the title compound (32.5 mg) as a yellow powder.

¹H-NMR (DMSO-d₆) δ: 1.05 (6H, d, J=6.8 Hz), 2.22 (3H, s), 2.36-2.55 (1H, m), 3.06 (3H, s), 3.47-3.62 (2H, m), 4.06 (2H, s), 4.69 (2H, t, J=6.4 Hz), 6.20-6.42 (2H, m), 6.64 (1H, d, J=3.0 Hz), 6.69-6.78 (1H, m), 6.93-7.03 (2H, m), 7.16 (1H, d, J=8.0 Hz), 7.31 (1H, t, J=8.0 Hz), 7.44 (1H, dd, J=2.5 Hz, 8.6 Hz), 7.53 (1H, d, J=2.5 Hz), 7.90 (1H, d, J=3.0 Hz), 8.67 (1H, s), 8.79 (1H, t, J=5.6 Hz), 9.85 (1H, s).

### Example E-28

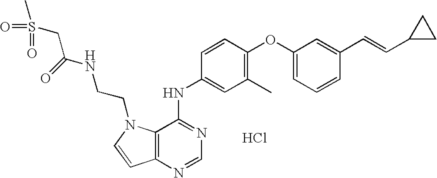

### Production of N-(2-{4-[(4-{3-[(E)-2-cyclopropylvinyl]phenoxy}-3-methylphenyl)amino]-5H-pyrrolo[3,2-d]pyrimidin-5-yl}ethyl)-2-(methylsulfonyl)acetamide hydrochloride

### (i) Production of 1-{3-[2-cyclopropylvinyl]phenoxy}-2-methyl-4-nitrobenzene

Using (cyclopropylmethyl)triphenylphosphonium bromide (1.86 g), tetrahydrofuran (30 mL), 1.6M n-butyllithium/hexane solution (3.5 mL) and 3-(2-methyl-4-nitrophenoxy)benzaldehyde (1.00 g) and in the same manner as in Example E-26(ii), the title compound (1.15 g, E/Z=about 1/2) was obtained as a yellow oil.

¹H-NMR (CDCl₃) δ: 0.43-0.56 (2H, m), 0.76-0.89 (2H, m), 1.50-1.63 (0.33H, m), 1.75-1.91 (0.67H, m), 2.40 (1H, s), 2.41 (2H, s), 5.10 (0.67H, dd, J=10 Hz, 11.5 Hz), 5.72 (0.33H, dd, J=8.9 Hz, 15.7 Hz), 6.30 (0.67H, d, J=11.5 Hz), 6.43 (0.33H, d, J=15.7 Hz), 6.76 (0.33H, d, J=9.0 Hz), 6.80 (0.67H, d, J=9.0 Hz), 6.79-6.84 (0.33H, m), 6.84-6.90 (0.67H, m), 6.96 (0.33H, t, J=1.9 Hz), 7.09 (0.67H, t, J=1.9 Hz), 7.13 (0.33H, d, J=8.0 Hz), 7.27 (0.67H, d, J=8.0 Hz), 7.30 (0.33H, t, J=8.0 Hz), 7.36 (0.67H, t, J=8.0 Hz), 7.93-8.02 (1H, m), 8.09 (1H, m).

### (ii) Production of 1-{3-[(E)-2-cyclopropylvinyl]phenoxy}-2-methyl-4-nitrobenzene

Using 1-{3-[2-cyclopropylvinyl]phenoxy}-2-methyl-4-nitrobenzene (1.14 g), tetrahydrofuran (25 mL), diphenyl disulfide (172 mg) and 2,2′-azobis(butyronitrile) (134 mg) and in the same manner as in Example E-26(iv), the title compound (734 mg) was obtained as an orange oil.

¹H-NMR (CDCl₃) δ: 0.47-0.55 (2H, m), 0.79-0.89 (2H, m), 1.50-1.63 (1H, m), 2.41 (3H, s), 5.73 (1H, dd, J=9.0 Hz, 15.7 Hz), 6.44 (1H, d, J=15.7 Hz), 6.77 (1H, d, J=9.0 Hz), 6.80-6.85 (1H, m), 6.97 (1H, t, J=2.0 Hz), 7.14 (1H, d, J=7.7 Hz), 7.30 (1H, t, J=7.7 Hz), 7.98 (1H, dd, J=2.2 Hz, 9.0 Hz), 8.15 (1H, d, J=2.2 Hz).

### (iii) Production of 4-{3-[(E)-2-cyclopropylvinyl]phenoxy}-3-methylaniline

Using 1-{3-[(E)-2-cyclopropylvinyl]phenoxy}-2-methyl-4-nitrobenzene (729 mg), reduced iron (712 mg), calcium chloride (156 mg) and ethanol (18 mL)/water (2 mL) and in the same manner as in Example E-18(v), the title compound (352 mg) was obtained as a brown oil.

¹H-NMR (CDCl₃) δ: 0.44-0.53 (2H, m), 0.75-0.85 (2H, m), 1.44-1.63 (1H, m), 2.10 (3H, s), 3.54 (2H, br s), 5.67 (1H, dd, J=9.0 Hz, 15.7 Hz), 6.39 (1H, d, J=15.7 Hz), 6.51 (1H, dd, J=2.7 Hz, 8.7 Hz), 6.59 (1H, d, J=2.7 Hz), 6.61-6.67 (1H, m), 6.75-6.82 (2H, m), 6.92 (1H, d, J=7.8 Hz), 7.14 (1H, t, J=7.8 Hz).

### (iv) Production of tert-butyl(2-{4-[(4-{3-[(E)-2-cyclopropylvinyl]phenoxy}-3-methylphenyl)amino]-5H-pyrrolo[3,2-d]pyrimidin-5-yl}ethyl)carbamate

Using tert-butyl[2-(4-chloro-5H-pyrrolo[3,2-d]pyrimidin-5-yl)ethyl]carbamate (352 mg), 4-{3-[(E)-2-cyclopropylvinyl]phenoxy}-3-methylaniline (347 mg) and isopropyl alcohol (15 mL) and in the same manner as in Example E-27(i), the title compound (540 mg) was obtained as a white solid.

¹H-NMR (CDCl₃) δ: 0.47-0.55 (2H, m), 0.75-0.87 (2H, m), 1.46 (9H, s), 1.50-1.61 (1H, m), 2.23 (3H, s), 3.43-3.56 (2H, m), 4.39-4.54 (2H, m), 4.98 (1H, t, J=5.9 Hz), 5.71 (1H, dd, J=9.0 Hz, 15.7 Hz), 6.41 (1H, d, J=15.7 Hz), 6.59 (1H, d, J=3.2 Hz), 6.75 (1H, dd, J=1.6 Hz, 8.2 Hz), 6.87-7.01 (3H, m), 7.12-7.23 (2H, m), 7.57-7.71 (2H, m), 8.31 (1H, br s), 8.50 (1H, s).

### (v) Production of 5-(2-aminoethyl)-N-(4-{3-[(E)-2-cyclopropylvinyl]phenoxy}-3-methylphenyl)-5H-pyrrolo[3,2-d]pyrimidin-4-amine dihydrochloride

Using tert-butyl(2-{4-[(4-{3-[(E)-2-cyclopropylvinyl]phenoxy}-3-methylphenyl)amino]-5H-pyrrolo[3,2-d]pyrimidin-5-yl}ethyl)carbamate (536 mg), 6N hydrochloric acid (1 mL) and ethanol (4 mL) and in the same manner as in Example E-27(ii), the title compound (399 mg) was obtained as a yellow solid.

¹H-NMR (DMSO-d₆) δ: 0.47-0.55 (2H, m), 0.74-0.84 (2H, m), 1.49-1.64 (1H, m), 2.21 (3H, s), 3.21-3.34 (2H. m), 5.02 (2H, t, J=6.2 Hz), 5.85 (1H, dd, J=9.4 Hz, 15.7 Hz), 6.44 (1H, d, J=15.7 Hz), 6.66-6.74 (2H, m), 6.90-6.99 (2H, m), 7.09 (1H, d, J=7.8 Hz); 7.27 (1H, t, J=7.8 Hz), 7.38 (1H, dd, J=2.6 Hz, 8.7. Hz), 7.48 (1H, d, J=2.5 Hz), 8.04 (1H, d, J=3.3 Hz), 8.37 (3H, br s), 8.66 (1H, s), 9.93 (1H, br s).

### (vi) Production of N-(2-{4-[(4-{3-[(E)-2-cyclopropylvinyl]phenoxy}-3-methylphenyl)amino]-5H-pyrrolo[3,2-d]pyrimidin-5-yl}ethyl)-2-(methylsulfonyl)acetamide hydrochloride

Using 5-(2-aminoethyl)-N-(4-{3-[(E)-2-cyclopropylvinyl]phenoxy}-3-methylphenyl)-5H-pyrrolo[3,2-d]pyrimidin-4-amine dihydrochloride (149 mg), methylsulfonylacetic acid (85.1 mg), tetrahydrofuran (0.8 mL)/N,N-dimethylformamide (0.8 mL), 1-hydroxybenzotriazole (118 mg), triethylamine (0.4 mL), 1-ethyl-3-(3-dimethylaminopropyl)carbodiimide hydrochloride (178 mg) and 4N hydrogen chloride/ethyl acetate solution and in the same manner as in Example E-27(iii), the title compound (123 mg) was obtained as yellow crystals.

¹H-NMR (DMSO-d₆) δ: 0.47-0.56 (2H, m), 0.74-0.86 (2H, m), 1.47-1.66 (1H, m), 2.22 (3H, s), 3.06 (3H, m), 3.49-3.63 (2H, m), 4.06 (2H, s), 4.69 (2H, t, J=6.2 Hz), 5.86 (1H, dd, J=9.2 Hz, 15.8 Hz), 6.44 (1H, d, J=15.8 Hz), 6.64 (1H, d, J=3.0 Hz), 6.70 (1H, dd, J=1.8 Hz, 8.0 Hz), 6.90-7.00 (2H, m), 7.10 (1H, d, J=8.0 Hz), 7.28 (1H, t, J=8.0 Hz), 7.43 (1H, dd, J=2.5 Hz, 8.6 Hz), 7.52 (1H, d, J=2.5 Hz), 7.90 (1H, d, J=3.0 Hz), 8.66 (1H, s), 8.79 (1H, t, J=5.7 Hz), 9.85 (1H, br s).

### Example E-29

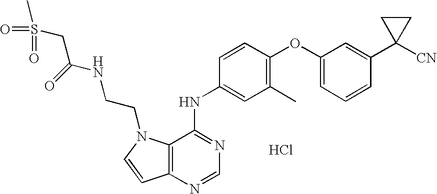

### Production of N-{2-[4-({4-[3-(1-cyanocyclopropyl)phenoxy]-3-methylphenyl}amino)-5H-pyrrolo[3,2-d]pyrimidin-5-yl]ethyl}-2-(methylsulfonyl)acetamide hydrochloride

### (i) Production of tert-butyl{2-[4-({4-[3-(1-cyanocyclopropyl)phenoxy]-3-methylphenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (350 mg), 1-[3-(4-amino-2-methylphenoxy)phenyl]cyclopropanecarbonitrile (351 mg) and isopropyl alcohol (10 mL) and in the same manner as in Example E-27(i), the title compound (590 mg) was obtained as a white powder.

¹H-NMR (CDCl₃) δ: 1.35-1.43 (2H, m), 1.47 (9H, s), 1.66-1.75 (2H, m), 2.21 (3H, s), 3.43-3.57 (2H, m), 4.41-4.53 (2H, m), 5.01 (1H, t, J=5.8 Hz), 6.59 (1H, d, J=3.2 Hz), 6.76-6.83 (1H, m), 6.86-6.95 (2H, m), 6.98-7.05 (1H, m), 7.16 (1H, d, J=3.2 Hz), 7.24 (1H, t, J=7.9 Hz), 7.58-7.76 (2H, m), 8.35 (1H, br s), 8.50 (1H, s).

### (ii) Production of 1-[3-(4-{[5-(2-aminoethyl)-5H-pyrrolo[3,2-d]pyrimidin-4-yl]amino}-2-methylphenoxy)phenyl]cyclopropanecarbonitrile dihydrochloride

Using tert-butyl{2-[4-({4-[3-(1-cyanocyclopropyl)phenoxy]-3-methylphenyl}amino)-5H-pyrrolo[3,2-d]pyrimidin-5-yl]ethyl}carbamate (586 mg), 6N hydrochloric acid (1 mL) and ethanol (6 mL) and in the same manner as in Example E-27(ii), the title compound (478 mg) was obtained as white crystals.

¹H-NMR (DMSO-d₆) δ: 1.47-1.58 (2H, m), 1.71-1.82 (2H, m), 2.21 (3H, s), 3.19-3.49 (2H, m), 4.98 (2H, t, J=6.1 Hz), 6.71 (1H, d, J=3.0 Hz), 6.80 (1H, dd, J=2.3 Hz, 7.7 Hz), 6.98 (1H, t, J=2.3 Hz), 7.01 (1H, d, J=8.7 Hz), 7.04-7.09 (1H, m), 7.39 (1H, t, d=7.7 Hz), 7.42 (1H, dd, J=2.3 Hz, 8.7 Hz), 7.51 (1H, d, J=2.3 Hz), 8.01 (1H, d, J=3.0 Hz), 8.26 (3H, br s), 8.66 (1H, s), 9.81 (1H, br s).

### (iii) Production of N-{2-[4-({4-[3-(1-cyanocyclopropyl)phenoxy]-3-methylphenyl}amino)-5H-pyrrolo[3,2-d]pyrimidin-5-yl]ethyl}-2-(methylsulfonyl)acetamide hydrochloride

Using 1-[3-(4-{[5-(2-aminoethyl)-5H-pyrrolo[3,2-d]pyrimidin-4-yl]amino}-2-methylphenoxy)phenyl]cyclopropanecarbonitrile dihydrochloride (150 mg), methylsulfonylacetic acid (65.2 mg), tetrahydrofuran (0.8 mL)/N,N-dimethylformamide (0.8 mL), 1-hydroxybenzotriazole (82.1 mg), triethylamine (0.4 mL), 1-ethyl-3-(3-dimethylaminopropyl)carbodiimide hydrochloride (117 mg) and 4N hydrogen chloride/ethyl acetate solution and in the same manner as in Example E-27(iii), the title compound (133 mg) was obtained as a pale-yellow powder.

¹H-NMR (DMSO-d₆) δ: 1.49-1.57 (2H, m), 1.73-1.81 (2H, m), 2.21 (3H, s), 3.05 (3H, s), 3.49-3.60 (2H, m), 4.06 (2H, s), 4.70 (2H, t, J=6.2 Hz), 6.63 (1H, d, J=3.0 Hz), 6.79 (1H, dd, J=2.5 Hz, 8.0 Hz), 6.98 (1H, t, J=2.5 Hz), 7.01 (1H, d, J=8.0 Hz), 7.03-7.09 (1H, m), 7.38 (1H, t, J=8.0 Hz), 7.46 (1H, dd, J=2.6 Hz, 8.7 Hz), 7.54 (1H, d, J=2.6 Hz), 7.90 (1H, d, J=3.0 Hz), 8.66 (1H, s), 8.81 (1H, t, J=5.5 Hz), 9.87 (1H, s).

### Example E-30

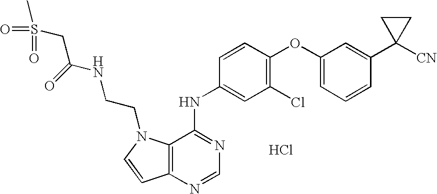

### Production of N-{2-[4-({3-chloro-4-[3-(1-cyanocyclopropyl)phenoxy]phenyl}amino)-5H-pyrrolo[3,2-d]pyrimidin-5-yl]ethyl}-2-(methylsulfonyl)acetamide hydrochloride

### (i) Production of 1-[3-(2-chloro-4-nitrophenoxy)phenyl]cyclopropanecarbonitrile

Using 3-chloro-4-fluoronitrobenzene (362 mg), 1-(3-hydroxyphenyl)cyclopropanecarbonitrile (350 mg), potassium carbonate (493 mg) and N,N-dimethylformamide (7 mL) and in the same manner as in Example E-18(iv), the title compound (650 mg) was obtained as a white powder.

¹H-NMR (CDCl₃) δ: 1.39-1.48 (2H, m), 1.74-1.84 (2H, m), 6.90 (1H, d, J=9 Hz), 6.95-7.01 (1H, m), 7.04 (1H, t, J=2.0 Hz), 7.20 (1H, d, J=8.0 Hz), 7.42 (1H, t, J=8.0 Hz), 8.08 (1H, dd, J=2.6 Hz, 9.0 Hz), 8.40 (1H, d, J=2.6 Hz).

### (ii) Production of 1-[3-(4-amino-2-chlorophenoxy)phenyl]cyclopropanecarbonitrile

Using 1-[3-(2-chloro-4-nitrophenoxy)phenyl]cyclopropanecarbonitrile (643 mg), reduced iron (570 mg), calcium chloride (113 mg) and ethanol (18 mL)/water (2 mL) and in the same manner as in Example E-18(v), the title compound (508 mg) was obtained as a yellow oil.

¹H-NMR (CDCl₃) δ: 1.34-1.41 (2H, m), 1.66-1.74 (2H, m), 3.68 (2H, br s), 6.57 (1H, dd, J=2.8 Hz, 8.5 Hz), 6.68-6.74 (1H, m), 6.78 (1H, d, J=2.8 Hz), 6.80 (1H, t, J=2.1 Hz), 6.88 (1H, d, J=8.5 Hz), 6.95-7.01 (1H, m), 7.23 (1H, t, J=8.0 Hz).

### (iii) Production of tert-butyl{2-[4-({3-chloro-4-[3-(1-cyanocyclopropyl)phenoxy]phenyl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (350 mg), 1-[3-(4-amino-2-chlorophenoxy)phenyl]cyclopropanecarbonitrile (349 mg) and isopropyl alcohol (8 mL) and in the same manner as in Example E-27(i), the title compound (632 mg) was obtained as a white powder.

¹H-NMR (CDCl₃) δ: 1.36-1.45 (2H, m), 1.50 (9H, s), 1.66-1.77 (2H, m), 3.43-3.57 (2H, m), 4.40-4.55 (2H, m), 5.07 (1H, t, J=5.5 Hz), 6.60 (1H, d, J=3.0 Hz), 6.84 (1H, dd, J=2.2 Hz, 8.0 Hz), 6.91 (1H, t, J=2.2 Hz), 7.03 (1H, d, J=9.1 Hz), 7.05-7.11 (1H, m), 7.18 (1H, d, J=3.0 Hz), 7.28 (1H, t, J=8.0 Hz), 7.87 (1H, dd, J=2.7 Hz, 9.1 Hz), 8.02 (1H, d, J=2.7 Hz), 8.51 (1H, s), 8.58 (1H, s).

### (iv) Production of 1-[3-(4-{[5-(2-aminoethyl)-5H-pyrrolo[3,2-d]pyrimidin-4-yl]amino}-2-chlorophenoxy)phenyl]cyclopropanecarbonitrile dihydrochloride

tert-Butyl{2-[4-({3-chloro-4-[3-(1-cyanocyclopropyl)phenoxy]phenyl}amino)-5H-pyrrolo[3,2-d]pyrimidin-5-yl]ethyl}carbamate (627 mg) was dissolved in a mixed solvent of ethanol (7 mL)/tetrahydrofuran (1 mL), 6N hydrochloric acid (1 mL) was added thereto, and the mixture was stirred at 50° C. for 14 hr. The reaction mixture was concentrated under reduced pressure, the residue was dissolved in concentrated hydrochloric acid (2 mL), and the mixture was stirred at room temperature for 10 min. Ethanol was added to the reaction mixture and the resulting precipitate was collected by filtration to give the title compound (433 mg) as a yellow powder.

¹H-NMR (DMSO-d₆) δ: 1.48-1.60 (2H, m), 1.72-1.84 (2H, m), 3.21-3.37 (2H, m), 5.01 (2H, t, J=6.4 Hz), 6.74 (1H, d, J=3.0 Hz), 6.85 (1H, dd, J=8.0 Hz, 2.2 Hz), 7.03 (1H, t, J=2.2 Hz), 7.08-7.15 (1H, m), 7.23 (1H, d, J=8.9 Hz), 7.42 (1H, t, J=8.0 Hz), 7.61 (1H, dd, J=2.5 Hz, 8.9 Hz), 7.90 (1H, d, J=2.5 Hz), 8.05 (1H, d, J=3.0 Hz), 8.29 (3H, br s), 8.71 (1H, s), 10.05 (1H, br s).

### (v) Production of N-{2-[4-({3-chloro-4-[3-(1-cyanocyclopropyl)phenoxy]phenyl}amino)-5H-pyrrolo[3,2-d]pyrimidin-5-yl]ethyl}-2-(methylsulfonyl)acetamide hydrochloride

Using 1-[3-(4-{[5-(2-aminoethyl)-5H-pyrrolo[3,2-d]pyrimidin-4-yl]amino}-2-chlorophenoxy)phenyl]cyclopropanecarbonitrile dihydrochloride (149 mg), methylsulfonylacetic acid (60.0 mg), tetrahydrofuran (0.8 mL)/N,N-dimethylformamide (0.8 mL), 1-hydroxybenzotriazole (77.4 mg), triethylamine (0.4 mL), 1-ethyl-3-(3-dimethylaminopropyl)carbodiimide hydrochloride (110 mg) and 4N hydrogen chloride/ethyl acetate solution and in the same manner as in Example E-27(iii), the title compound (131 mg) was obtained as a white powder.

¹H-NMR (DMSO-d₆) δ: 1.50-1.60 (2H, m), 1.74-1.83 (2H, m), 3.06 (3H, s), 3.54 (2H, q, J=6.0 Hz), 4.06 (2H, s), 4.70 (2H, t, J=6.0 Hz), 6.66 (1H, d, J=3.0 Hz), 6.85 (1H, dd, J=2.2 Hz, 8.0 Hz), 7.02 (1H, t, J=2.2 Hz), 7.08-7.14 (1H, m), 7.24 (1H, d, J=9.0 Hz), 7.41 (1H, t, J=8.0 Hz), 7.64 (1H, dd, J=2.5 Hz, 9.0 Hz), 7.92 (1H, d, J=2.5 Hz), 7.93 (1H, d, J=3.0 Hz), 8.71 (1H, s), 8.77 (1H, t, J=5.8 Hz), 9.91 (1H, br s).

### Example E-31

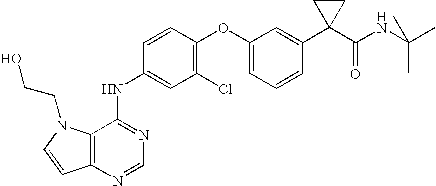

### Production of N-(tert-butyl)-1-[3-(2-chloro-4-{[5-(2-hydroxyethyl)-5H-pyrrolo[3,2-d]pyrimidin-4-yl]amino}phenoxy)phenyl]cyclopropanecarboxamide

### (i) Production of N-(tert-butyl)-1-(3-methoxyphenyl)cyclopropanecarboxamide

1-(3-Methoxyphenyl)cyclopropanecarboxylic acid (400 mg) was dissolved in tetrahydrofuran (6 mL), a catalytic amount of N,N-dimethylformamide and thionyl chloride (0.4 mL) were added thereto, and the mixture was stirred at room temperature for 6 hr. The reaction mixture was concentrated under reduced pressure, and the residue was dissolved in tetrahydrofuran (4 mL). The solution was added dropwise to a mixture cooled to 0° C. of tert-butylamine (0.25 mL), triethylamine (1 mL) and tetrahydrofuran (3 mL), and the mixture was stirred at room temperature for 2 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (hexane:ethyl acetate=95:5→67:33) to give the title compound (477 mg) as a pale-yellow oil.

¹H-NMR (CDCl₃) δ: 0.97 (2H, q, J=3.7 Hz), 1.22 (9H, s), 1.52 (2H, q, J=3.7 Hz), 3.82 (3H, s), 5.22 (1H, br s), 6.81-6.87 (1H, m), 6.90-6.94 (1H, m), 6.95-7.01 (1H, m), 7.27 (1H, t, J=7.9 Hz).

### (ii) Production of N-(tert-butyl)-1-(3-hydroxyphenyl)cyclopropanecarboxamide

Using N-(tert-butyl)-1-(3-methoxyphenyl)cyclopropanecarboxamide (470 mg), benzotrifluoride (10 mL) and 1N boron tribromide/dichloromethane solution (4 mL) and in the same manner as in Example E-18(iii), the title compound (245 mg) was obtained as a white powder.

¹H-NMR (CDCl₃) δ: 0.95-1.04 (2H, m), 1.22 (9H, s), 1.50-1.58 (2H, m), 5.36 (1H, br s), 6.13 (1H, br s), 6.76-6.88 (2H, m), 6.90-6.98 (1H, m), 7.19-7.29 (1H, m).

### (iii) Production of N-(tert-butyl)-1-[3-(2-chloro-4-nitrophenoxy)phenyl]cyclopropanecarboxamide

Using N-(tert-butyl)-1-(3-hydroxyphenyl)cyclopropanecarboxamide (242 mg), 3-chloro-4-fluoronitrobenzene (237 mg), potassium carbonate (223 mg) and N,N-dimethylformamide (5 mL) and in the same manner as in Example E-18(iv), the title compound (397 mg) was obtained as white crystals.

¹H-NMR (CDCl₃) δ: 0.96-1.01 (2H, m), 1.23 (9H, s), 1.52-1.59 (2H, m), 5.08 (1H, br s), 6.88 (1H, d, J=9.1 Hz), 6.98-7.04 (1H, m), 7.08-7.13 (1H, m), 7.27-7.32 (1H, m), 7.43 (1H, t, J=8.0 Hz), 8.07 (1H, dd, J=2.8 Hz, 9.1 Hz), 8.40 (1H, d, J=2.8 Hz).

### (iv) Production of 1-[3-(4-amino-2-chlorophenoxy)phenyl]-N-(tert-butyl)cyclopropanecarboxamide

N-(tert-butyl)-1-[3-(2-chloro-4-nitrophenoxy)phenyl]cyclopropanecarboxamide (392 mg) was dissolved in a mixed solvent of methanol (7.5 mL)/ethyl acetate (7.5 mL), 5% platinum-activated carbon (44.4 mg) was added thereto and the mixture was stirred under a hydrogen atmosphere at room temperature for 1.5 hr. The reaction mixture was filtered through celite, and the filtrate was concentrated under reduced pressure. The residue was separated and purified by silica gel column chromatography (hexane:ethyl acetate=95:5→50:50) to give the title compound (324 mg) as a colorless oil.

¹H-NMR (CDCl₃) δ: 0.91-0.98 (2H, m), 1.21 (9H, s), 1.45-1.52 (2H, m), 3.70 (2H, s), 5.17 (1H, br s), 6.59 (1H, dd, J=2.7 Hz, 8.6 Hz), 6.79 (1H, d, J=2.7 Hz), 6.81-6.87 (2H, m), 6.91 (1H, d, J=8.6 Hz), 6.99-7.06 (1H, m), 7.21-7.31 (1H, m).

### (v) Production of N-(tert-butyl)-1-[3-(2-chloro-4-{[5-(2-hydroxyethyl)-5H-pyrrolo[3,2-d]pyrimidin-4-yl]amino}phenoxy)phenyl]cyclopropanecarboxamide

Using 2-(4-chloro-5H-pyrrolo[3,2-d]pyrimidin-5-yl)ethyl benzoate (120 mg), 1-[3-(4-amino-2-chlorophenoxy)phenyl]-N-(tert-butyl)cyclopropanecarboxamide (160 mg), isopropyl alcohol (4 mL) and 1N aqueous sodium hydroxide solution (1.5 mL) and in the same manner as in Example E-18(vi), the title compound (155 mg) was obtained as a white powder.

¹H-NMR (CDCl₃) δ: 0.96 (2H, q, J=3.7 Hz), 1.21 (9H, s), 1.49 (2H, q, J=3.7 Hz), 4.14-4.22 (2H, m), 4.37-4.46 (2H, m), 5.19 (1H, s), 6.06 (1H, br s), 6.19 (1H, d, J=3.2 Hz), 6.89-6.98 (2H, m), 7.02 (1H, d, J=3.2 Hz), 7.04-7.11 (2H, m), 7.31 (1H, t, J=7.8 Hz), 7.52 (1H, dd, J=2.5 Hz, 8.7 Hz), 7.81 (1H, d, J=2.5 Hz), 8.29 (1H, s), 9.58 (1H, s).

### Example G-1

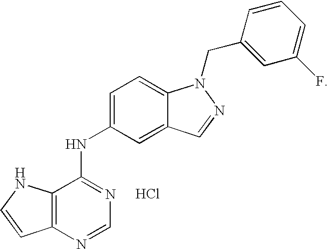

### Production of N-[1-(3-fluorobenzyl)-1H-indazol-5-yl]-5H-pyrrolo[3,2-d]pyrimidin-4-amine hydrochloride

A solution of 4-chloro-5H-pyrrolo[3,2-d]pyrimidine (153 mg) and 1-(3-fluorobenzyl)-1H-indazol-5-amine (362 mg) in N,N-dimethylformamide (2 mL) was stirred at 120° C. for 2.5 hr. The reaction mixture was cooled to room temperature, ethyl acetate (30 mL) was added thereto and the mixture was stirred at room temperature for 30 min. The resulting crystals were collected by filtration, washed with ethyl acetate and dried under reduced pressure to give the title compound (361 mg) as crystals.

¹H-NMR (DMSO-d₆) δ: 5.70 (2H, s), 6.57 (1H, d, J=2 Hz), 7.00-7.20 (3H, m), 7.36 (1H, m), 7.70-7.90 (3H, m), 8.17 (1H, s), 8.41 (1H, m), 8.59 (1H, s), 10.97 (1H, br s), 12.58 (1H, br s).

### Example G-2

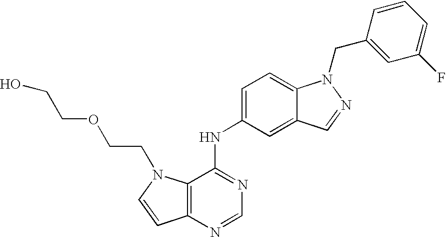

### Production of 2-[2-(4-{[1-(3-fluorobenzyl)-1H-indazol-5-yl]amino}-5H-pyrrolo[3,2-d]pyrimidin-5-yl)ethoxy]ethanol

A mixture of 2-[2-(4-chloro-5H-pyrrolo[3,2-d]pyrimidin-5-yl)ethoxy]ethyl benzoate (150 mg), 1-(3-fluorobenzyl)-1H-indazol-5-amine (156 mg) and 1-methyl-2-pyrrolidone (0.863 mL) was stirred with heating at 140° C. for 2 hr. The reaction mixture was diluted with ethyl acetate (80 mL), and the organic layer was washed with aqueous sodium bicarbonate (30 mL), dried over anhydrous magnesium sulfate and concentrated under reduced pressure. The residue was purified by silica gel column chromatography (hexane:ethyl acetate=70:30→0:100), and the objective fractions were concentrated under reduced pressure. The obtained residue was dissolved in methanol (1.89 mL), 1N aqueous sodium hydroxide solution (0.433 mL) was added, and the mixture was stirred at room temperature for 2 hr. 1N hydrochloric acid (0.433 mL) was added to the mixture, and the mixture was diluted with ethyl acetate (80 mL), and the organic layer was washed with saturated brine (30 mL), dried over anhydrous magnesium sulfate and concentrated under reduced pressure. The residue was subjected to silica gel column chromatography (ethyl acetate:methanol=100:0→90:10) and crystallized from isopropyl ether to give the title compound (153 mg) as crystals.

¹H-NMR (DMSO-d₆) δ: 3.50 (4H, br s), 3.84 (2H, t, J=4 Hz), 4.64 (2H, t, J=4 Hz), 4.69 (1H, m), 5.69 (2H, s), 6.47 (1H, d, J=3 Hz), 7.00-7.20 (3H, m), 7.36 (1H, m), 7.53 (1H, d, J=9 Hz), 7.60-7.70 (2H, m), 8.05 (1H, s), 8.09 (1H, s), 8.23 (1H, s), 8.76 (1H, br s).

### Example G-3

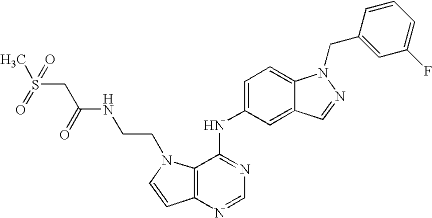

### Production of N-[2-(4-{[1-(3-fluorobenzyl)-1H-indazol-5-yl]amino}-5H-pyrrolo[3,2-d]pyrimidin-5-yl)ethyl]-2-(methylsulfonyl)acetamide

### (i) Production of tert-butyl[2-(4-{[1-(3-fluorobenzyl)-1H-indazol-5-yl]amino}-5H-pyrrolo[3,2-d]pyrimidin-5-yl)ethyl]carbamate

A solution of tert-butyl[2-(4-chloro-5H-pyrrolo[3,2-d]pyrimidin-5-yl)ethyl]carbamate (0.5 g) and 1-(3-fluorobenzyl)-1H-indazol-5-amine (608 mg) in isopropyl alcohol (5 mL) was stirred at 80° C. for 12 hr. Aqueous sodium bicarbonate (30 mL) was added to the reaction mixture, and the mixture was extracted with ethyl acetate (80 mL). The organic layer was washed with saturated brine and dried over anhydrous magnesium sulfate. After concentration under reduced pressure, the residue was separated and purified by silica gel column chromatography (eluent, hexane:ethyl acetate=7:3→Methyl acetate) to give the title compound (820 mg) as colorless crystals.

¹H-NMR (CDCl₃) δ: 1.44 (9H, s), 3.50 (2H, m), 4.46 (2H,m), 5.07 (1H, m), 5.58 (2H, s), 6.57 (1H, d, J=3 Hz), 6.80-7.00 (3H, m), 7.14 (1H, d, J=3 Hz), 7.20-7.40 (2H, m), 7.74 (1H, dd, J=2 Hz, 9 Hz), 7.99 (1H, s), 8.05 (1H, d, J=1 Hz), 8.45 (1H, s), 8.53 (1H, br s).

### (ii) Production of 5-(2-aminoethyl)-N-[1-(3-fluorobenzyl)-1H-indazol-5-yl]-5H-pyrrolo[3,2-d]pyrimidin-4-amine trihydrochloride

A mixture of tert-butyl[2-(4-{[1-(3-fluorobenzyl)-1H-indazol-5-yl]amino}-5H-pyrrolo[3,2-d]pyrimidin-5-yl)ethyl]carbamate (780 mg), 2N hydrochloric acid (11.1 mL) and tetrahydrofuran (22.2 mL) was stirred at 60° C. for 20 hr. The solvent was evaporated under reduced pressure, ethanol was added to the mixture, and the mixture was further concentrated. The precipitated powder was collected by filtration and washed with isopropyl ether to give the title compound (668 mg) as a pale-yellow powder.

¹H-NMR (DMSO-d₆) δ: 3.32 (2H, m), 5.08 (2H, m), 5.74 (2H, s), 6.72 (1H, d, J=3 Hz), 7.00-7.20 (3H, m), 7.38 (1H, m), 7.52 (1H, dd, J=2 Hz, 9 Hz), 7.80 (1H, d, J=9 Hz), 7.91 (1H, d, J=2 Hz), 8.07 (1H, m), 8.20 (1H, s), 8.45 (3H, br s), 8.60 (1H, s).

### (iii) Production of N-[2-(4-{[1-(3-fluorobenzyl)-1H-indazol-5-yl]amino}-5H-pyrrolo[3,2-d]Pyrimidin-5-yl)ethyl]-2-(methylsulfonyl)acetamide

A mixture of 5-(2-aminoethyl)-N-[1-(3-fluorobenzyl)-1H-indazol-5-yl]-5H-pyrrolo[3,2-d]pyrimidin-4-amine trihydrochloride (183 mg), methylsulfonylacetic acid (74.2 mg), 1-ethyl-3-(3-dimethylaminopropyl)carbodiimide hydrochloride (103 mg), 1-hydroxybenzotriazole (72.5 mg), triethylamine (0.15 mL) and N,N-dimethylformamide (6.9 mL) was stirred at room temperature for 16 hr. Water was added to the reaction mixture and the mixture was extracted with ethyl acetate. The organic layer was washed successively with water and saturated brine, and dried over anhydrous magnesium sulfate. After concentration under reduced pressure, the residue was separated and purified by basic silica gel column chromatography (eluent, ethyl acetate→ethyl acetate:methanol=90:10) and crystallized from isopropyl ether to give the title compound (134 mg) as crystals.

¹H-NMR (DMSO-d₆) δ: 3.07 (3H, s), 3.48 (2H, q, J=6 Hz), 4.04 (2H, s), 4.56 (2H, t, J=6 Hz), 5.69 (2H, s), 6.45 (1H, d, J=3 Hz), 7.00-7.20 (3H, m), 7.30-7.40 (1H, m), 7.57 (2H, m), 7.68 (1H, d, J=9 Hz), 7.95 (1H, m), 8.10 (1H, s), 8.21 (1H, s), 8.58 (1H, br s), 8.65 (1H, t, J=6 Hz).

### Example G-4

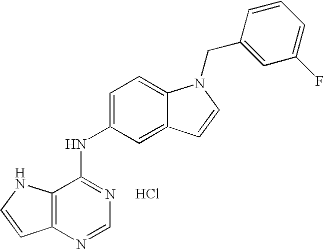

### Production of N-[1-(3-fluorobenzyl)-1H-indol-5-yl]-5H-pyrrolo[3,2-d]pyrimidin-4-amine hydrochloride

A solution of 4-chloro-5H-pyrrolo[3,2-d]pyrimidine (153 mg) and 1-(3-fluorobenzyl)-1H-indol-5-amine (360 mg) in N,N-dimethylformamide (2 mL) was stirred at 120° C. for 2 hr. The reaction mixture was cooled to room temperature, ethyl acetate (30 mL) was added thereto, and the mixture was stirred at room temperature for 30 min. The resulting crystals were collected by filtration, washed with ethyl acetate and dried under reduced pressure to give the title compound (377 mg) as crystals.

¹H-NMR (DMSO-d₆) δ: 5.46 (2H, s), 6.53 (2H, d, J=3 Hz), 7.05 (3H, m), 7.36 (1H, m), 7.49 (2H, m), 7.56 (1H, d, J=3 Hz), 7.79 (1H, br s), 8.16 (1H, br s), 8.51 (1H, s), 10.61 (1H, br s), 12.45 (1H, br s).

### Example G-5

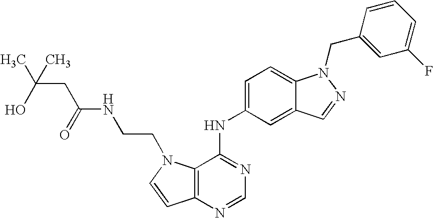

### Production of N-[2-(4-{[1-(3-fluorobenzyl)-1H-indazol-5-yl]amino}-5H-pyrrolo[3,2-d]pyrimidin-5-yl)ethyl]-3-hydroxy-3-methylbutanamide

A mixture of 5-(2-aminoethyl)-N-[1-(3-fluorobenzyl)-1H-indazol-5-yl]-5H-pyrrolo[3,2-d]pyrimidin-4-amine trihydrochloride (183 mg), 3-hydroxy-3-methylbutanoic acid (0.058 mL), 1-ethyl-3-(3-dimethylaminopropyl)carbodiimide hydrochloride (103 mg), 1-hydroxybenzotriazole (72.5 mg), triethylamine (0.15 mL) and N,N-dimethylformamide (6.9 mL) was stirred at room temperature for 4 hr. Water was added to the reaction mixture and the mixture was extracted with ethyl acetate. The organic layer was washed successively with water and saturated brine, and dried over anhydrous magnesium sulfate. After concentration under reduced pressure, the residue was separated and purified by basic silica gel column chromatography (eluent, ethyl acetate→ethyl acetate:methanol=85:15) and crystallized from isopropyl ether to give the title compound (100 mg) as crystals.

¹H-NMR (DMSO-d₆) δ: 1.12 (6H, s), 2.20 (2H, s), 3.44 (2H, q, J=7 Hz), 4.52 (2H, t, J=7 Hz), 4.69 (1H, s), 5.69 (2H, s), 6.44 (1H, d, J=3 Hz), 7.00-7.20 (3H, m), 7.30-7.40 (1H, m), 7.50-7.70 (3H, m), 8.00 (1H, d, J=2 Hz), 8.09 (1H, s), 8.20 (1H, s), 8.23 (1H, t, J=7 Hz), 8.76 (1H, br s).

### Example G-6

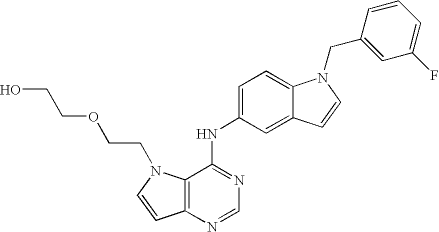

### Production of 2-[2-(4-{[1-(3-fluorobenzyl)-1H-indol-5-yl]amino}-5H-pyrrolo[3,2-d]pyrimidin-5-yl)ethoxy]ethanol

A mixture of 2-[2-(4-chloro-5H-pyrrolo[3,2-d]pyrimidin-5-yl)ethoxy]ethyl benzoate (150 mg), 1-(3-fluorobenzyl)-1H-indol-5-amine (155 mg) and 1-methyl-2-pyrrolidone (0.863 mL) was stirred with heating at 140° C. for 2 hr. The reaction mixture was diluted with ethyl acetate (80 mL) and washed with aqueous sodium bicarbonate (30 mL). The organic layer was separated, dried over anhydrous magnesium sulfate, and concentrated under reduced pressure. The residue was purified by silica gel column chromatography (hexane:ethyl acetate=70:30→0:100→ethyl acetate:methanol=95:5), and the objective fractions were concentrated under reduced pressure. The obtained residue was dissolved in, methanol (1.89 mL), 1N aqueous sodium hydroxide solution (0.433 mL) was added thereto, and the mixture was stirred at room temperature for 2 hr. 1N hydrochloric acid (0.433 mL) was added to the mixture, and the mixture was diluted with ethyl acetate (80 mL). The organic layer was washed with saturated brine (30 mL), dried over magnesium sulfate and concentrated under reduced pressure. The residue was subjected to silica gel column chromatography (ethyl acetate:methanol=100:0→90:10) and crystallized from isopropyl ether/ethyl acetate to give the title compound (107 mg) as crystals.

¹H-NMR (DMSO-d₆) δ: 3.49 (4H, br s), 3.83 (2H, t, J=4 Hz), 4.61 (2H, t, J=4 Hz), 4.67 (1H, t, J=4 Hz), 5.45 (2H, s), 6.44 (1H, dd, J=1.5 Hz, 3 Hz), 6.47 (1H, d, J=3 Hz), 6.90-7.10 (3H, m), 7.25 (1H, d, J=9 Hz), 7.3-7.5 (2H, m), 7.51 (1H, d, J=3 Hz), 7.59 (1H, d, J=3 Hz), 7.81 (1H, s), 8.17 (1H, s), 8.58 (1H, br s).

### Example G-7

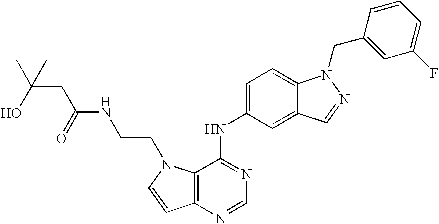

### Production of N-[2-(4-{[1-(3-fluorobenzyl)-1H-indol-5-yl]amino}-5H-pyrrolo[3,2-d]pyrimidin-5-yl)ethyl]-3-hydroxy-3-methylbutanamide

### (i) Production of tert-butyl[2-(4-{[1-(3-fluorobenzyl)-1H-indol-5-yl]amino}-5H-pyrrolo[3,2-d]pyrimidin-5-yl)ethyl]carbamate

A solution of tert-butyl[2-(4-chloro-5H-pyrrolo[3,2-d]pyrimidin-5-yl)ethyl]carbamate (0.5 g) and 1-(3-fluorobenzyl)-1H-indol-5-amine (606 mg) in isopropyl alcohol (5 mL) was stirred at 80° C. for 12 hr. Aqueous sodium bicarbonate (30 mL) was added to the reaction mixture, and the mixture was extracted with ethyl acetate (80 mL). The organic layer was washed with saturated brine and dried over anhydrous magnesium sulfate. After concentration under reduced pressure, the residue was separated and purified by silica gel column chromatography (eluent, hexane:ethyl acetate=7:3→Methyl acetate→ethyl acetate:methanol=95:5) to give the title compound (840 mg) as colorless crystals.

¹H-NMR (CDCl₃) δ: 1.42 (9H, s), 3.45 (2H, m), 4.38 (2H,t, J=7 Hz), 5.17 (1H, m), 5.29 (2H, s), 6.51 (1H, dd, J=1 Hz, 3 Hz), 6.53 (1H, d, J=3 Hz), 6.78 (1H, d, J=9 Hz), 6.80-7.00 (2H, m), 7.10 (2H, t, J=3 Hz), 7.18-7.30 (2H, m), 7.47 (1H, d, J=9 Hz), 7.89 (1H, br s), 8.20 (1H, br s), 8.43 (1H, s).

### (ii) Production of 5-(2-aminoethyl)-N-[1-(3-fluorobenzyl)-1H-indol-5-yl]-5H-pyrrolo[3,2-d]pyrimidin-4-amine dihydrochloride

A mixture of tert-butyl[2-(4-{[1-(3-fluorobenzyl)-1H-indol-5-yl]amino}-5H-pyrrolo[3,2-d]pyrimidin-5-yl)ethyl]carbamate (830 mg), 2N hydrochloric acid (11.8 mL) and tetrahydrofuran (23.6 mL) was stirred at 60° C. for 20 hr. The solvent was evaporated under reduced pressure, ethanol was added to the mixture, and the mixture was further concentrated. The precipitated powder was collected by filtration and washed with isopropyl ether to give the title compound (828 mg) as a pale-yellow powder.

¹H-NMR (DMSO-d₆) δ: 3.27 (2H, m), 5.07 (2H, m), 5.43 (2H, s), 6.50-6.70 (2H, m), 6.80-7.30 (4H, m), 7.37 (1H, m), 7.68 (1H, m), 7.86 (1H, s), 8.04 (1H, d, J=3 Hz), 8.44 (1H, s), 8.50 (3H, br s), 8.55 (1H, s), 10.01 (1H, br s).

### (iii) Production of N-[2-(4-{[1-(3-fluorobenzyl)-1H-indol-5-yl]amino}-5H-pyrrolo[3,2-d]pyrimidin-5-yl)ethyl]-3-hydroxy-3-methylbutanamide

A mixture of 5-(2-aminoethyl)-N-[1-(3-fluorobenzyl)-1H-indol-5-yl]-5H-pyrrolo[3,2-d]pyrimidin-4-amine dihydrochloride (183 mg), 3-hydroxy-3-methylbutanoic acid (0.058 mL), 1-ethyl-3-(3-dimethylaminopropyl)carbodiimide hydrochloride (103 mg), 1-hydroxybenzotriazole (72.5 mg), triethylamine (0.15 mL) and N,N-dimethylformamide (6.9 mL) was stirred at room temperature for 16 hr. Water was added to the reaction mixture and the mixture was extracted with ethyl acetate. The organic layer was washed successively with water and saturated brine, and dried over anhydrous magnesium sulfate. After concentration under reduced pressure, the residue was separated and purified by basic silica gel column chromatography (eluent, ethyl acetate→ethyl acetate:methanol=85:15) and crystallized from isopropyl ether to give the title compound (43 mg) as crystals.

¹H-NMR (DMSO-d₆) δ: 1.12 (6H, s), 2.20 (2H, s), 3.44 (2H, q, J=7 Hz), 4.50 (2H, t, J=7 Hz), 4.70 (1H, br s), 5.46 (2H, s), 6.41 (1H, d, J=3 Hz), 6.47 (1H, d, J=3 Hz), 6.9-7.2 (3H, m), 7.20-7.50 (3H, m), 7.52 (2H, t, J=3 Hz), 7.76 (1H, s), 8.15 (1H, s), 8.19 (1H, t, J=6 Hz), 8.57 (1H, br s).

### Example G-8

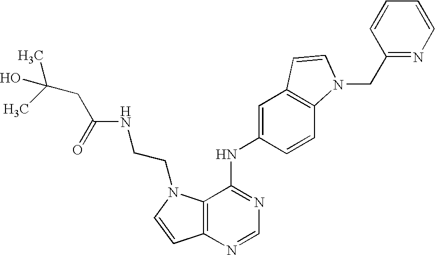

### Production of 3-hydroxy-3-methyl-N-[2-(4-{[1-(pyridin-2-ylmethyl)-1H-indol-5-yl]amino}-5H-pyrrolo[3,2-d]pyrimidin-5-yl)ethyl]butanamide

### (i) Production of 5-nitro-1-(pyridin-2-ylmethyl)-1H-indole

To a solution of 5-nitroindole (1.62 g) and 2-(chloromethyl)pyridine hydrochloride (1.80 g) in N,N-dimethylformamide (20 mL) was added potassium carbonate (3.46 g) under ice-cooling, and the mixture was stirred at room temperature for 16 hr. Under ice-cooling, water was added to the reaction mixture, and the mixture was extracted with ethyl acetate. The organic layer was washed with brine, dried over anhydrous magnesium sulfate and concentrated under reduced pressure. The residue was recrystallized from ethyl acetate/diisopropyl ether to give the title compound (2.05 g) as yellow crystals.

¹H-NMR (CDCl₃) δ: 5.49 (2H, s), 6.75-6.80 (2H, m), 7.15-7.25 (1H, m), 7.32 (1H, d, J=9.0 Hz), 7.36 (1H, d, J=3.3 Hz), 7.58 (1H, dt, J=2.1 Hz, 7.8 Hz), 8.07 (1H, dd, J=2.1 Hz, 9.0 Hz), 8.55-8.65 (2H, m).

### (ii) Production of 1-(pyridin-2-ylmethyl)-1H-indol-5-amine

To a solution of 5-nitro-1-(pyridin-2-ylmethyl)-1H-indole (507 mg) in ethyl acetate (10 mL)/methanol (2 mL) was added 5% platinum-activated carbon (84.5 mg) under a nitrogen atmosphere. The reaction mixture was stirred under a hydrogen atmosphere at room temperature for 6 hr. The platinum-activated carbon was filtered off, and the filtrate was concentrated under reduced pressure. The residue was separated and purified by silica gel column chromatography (eluent, ethyl acetate:hexane=70:30→100:0) and further recrystallized from ethyl acetate/hexane to give the title compound (357 mg) as a white-peach powder.

¹H-NMR (CDCl₃) δ: 3.48 (2H, br s), 5.38 (2H, s), 6.39 (1H, d, J=3.0 HZ), 6.55-6.70 (2H, m), 6.94 (1H, s), 7.03 (1H, d, J=8.7 Hz), 7.10-7.20 (2H, m), 7.49 (1H, t, J=7.8 Hz), 8.57 (1H, d, J=4.2 Hz).

### (iii) Production of tert-butyl[2-(4-{[1-(pyridin-2-ylmethyl)-1H-indol-5-yl]amino}-5H-pyrrolo[3,2-d]pyrimidin-5-yl)ethyl]carbamate

A mixture of tert-butyl[2-(4-chloro-5H-pyrrolo[3,2-d]pyrimidin-5-yl)ethyl]carbamate (297 mg), 1-(pyridin-2-ylmethyl)-1H-indol-5-amine (246 mg) and isopropyl alcohol (5.0 mL) was stirred at 80° C. for 16 hr. Under ice-cooling, aqueous sodium bicarbonate was added to the reaction mixture and the mixture was extracted with ethyl acetate. The organic layer was washed with brine, dried over anhydrous magnesium sulfate and concentrated under reduced pressure. The residue was separated and purified by silica gel column chromatography (eluent, ethyl acetate:methanol=100:0→90:10) to give the title compound (498 mg) as a pale-purple powder.

¹H-NMR (CDCl₃) δ: 1.43 (9H, s), 3.45-3.55 (2H, m), 4.35-4.45 (2H, m), 4.9-5.0 (1H, m), 5.45 (2H, s), 6.53 (1H, d, J=3.0 Hz), 6.55 (1H, d, J=3.0 Hz), 6.71 (1H, d, J=8.1 Hz), 7.10-7.30 (4H, m), 7.40-7.50 (1H, m), 7.52 (1H, dt, J=1.8, 7.8 Hz), 7.90 (1H, s), 8.17 (1H, br s), 8.44 (1H, s), 8.58 (1H, d, J=4.2 Hz).

### (iv) Production of 5-(2-aminoethyl)-N-[1-(pyridin-2-ylmethyl)-1H-indol-5-yl]-5H-pyrrolo[3,2-d]pyrimidin-4-amine tetrahydrochloride

A mixture of tert-butyl[2-(4-{[1-(pyridin-2-ylmethyl)-1H-indol-5-yl]amino}-5H-pyrrolo[3,2-d]pyrimidin-5-yl)ethyl]carbamate (439 mg) and 10% (W/W) hydrochloric acid/methanol (5 mL) was stirred at 65° C. for 18 hr. The reaction mixture was concentrated under reduced pressure, and the precipitate was collected by filtration and washed with diethyl ether to give the title compound (422 mg) as pale-green crystals.

¹H-NMR (DMSO-d₆) δ: 3.2-3.4 (2H, m), 4.90-5.10 (2H, m), 5.63 (2H, s), 6.57 (1H, d, J=3.3 Hz), 6.69 (1H, d, J=3.3 Hz), 7.08 (1H, d, J=7.2 Hz), 7.20 (1H, d, J=8.7 Hz), 7.35-7.45 (1H, m), 7.52 (1H, d, J=8.7 Hz), 7.61 (1H, d, J=3.3 Hz), 7.67 (1H, s), 7.85-7.95 (1H, m), 8.01 (1H, d, J=2.7 Hz), 8.25-8.4 (3H, m), 8.56 (1H, s), 8.61 (1H, d, J 5.1 Hz), 9.97 (1H, s).

### (v) Production of 3-hydroxy-3-methyl-N-[2-(4-{[1-(pyridin-2-ylmethyl)-1H-indol-5-yl]amino}-5H-pyrrolo[3,2-d]pyrimidin-5-yl)ethyl]butanamide

To a solution of 5-(2-aminoethyl)-N-[1-(pyridin-2-ylmethyl)-1H-indol-5-yl]-5H-pyrrolo[3,2-d]pyrimidin-4-amine tetrahydrochloride (200 mg), 3-hydroxy-3-methylbutanoic acid (67 mg) and 1-hydroxybenzotriazole (85 mg) in N,N-dimethylformamide (5.0 mL), were added triethylamine (0.52 mL) and 1-ethyl-3-(3-dimethylaminopropyl)carbodiimide hydrochloride (120 mg) under ice-cooling, and the mixture was stirred at room temperature for 16 hr. Water was added to the reaction mixture and the mixture was extracted with ethyl acetate. The organic layer, was washed with brine and dried over anhydrous magnesium sulfate. After concentration under reduced pressure, the residue was separated and purified by silica gel column chromatography (eluent, ethyl acetate:methanol=100:0→70:30) and further recrystallized from ethyl acetate/diisopropyl ether to give the title compound (85.3 mg) as colorless crystals.

¹H-NMR (DMSO-d₆) δ: 1.26 (6H, s), 2.37 (2H, s), 3.55-3.7 (2H, m), 4.35-4.45 (2H, m), 5.45 (2H, s), 6.55. (2H, dd, J=3.3 Hz, 7.8 Hz), 6.73 (1H, d, J=7.8 Hz), 6.75-6.85 (1H, m), 7.1-7.25 (3H, m), 7.39 (1H, dd, J=1.8 Hz, 8.7 Hz), 7.53 (1H, dt, J=1.8 Hz, 7.8 Hz), 7.87 (1H, d, J=1.5 Hz), 8.22 (1H, s), 8.43 (1H, s), 8.58 (1H, d, J=4.8 Hz).

### Example G-9

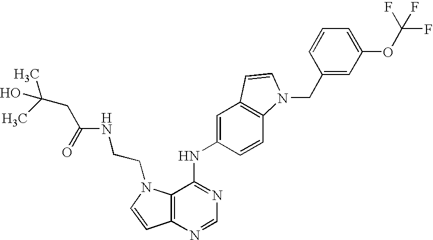

### Production of 3-hydroxy-3-methyl-N-{2-[4-({1-[3-(trifluoromethoxy)benzyl]-1H-indol-5-yl}amino)-5H-pyrrolo[3,2-d]pyrimidin-5-yl]ethyl}butanamide

### (i) Production of 5-nitro-1-[3-(trifluoromethoxy)benzyl]-1H-indole

Using 5-nitroindole (538 mg), 1-(bromomethyl)-3-(trifluoromethoxy)benzene (0.96 g), potassium carbonate (551 mg) and N,N-dimethylformamide (8.0 mL) and in the same manner as in Example G-8(i), the title compound (0.95 g) was obtained as a pale-yellow powder.

¹H-NMR (CDCl₃) δ: 5.39 (2H, s), 6.76 (1H, d, J=3.3 Hz), 6.90-7.00 (2H, m), 7.15 (1H, d, J=8.1 Hz), 7.20-7.30 (2H, m), 7.35 (1H, t, J=8.1 Hz), 8.09 (1H, dd, J=2.1 Hz, 9.0 Hz), 8.62 (1H, d, J=2.4 Hz).

### (ii) Production of 1-[3-(trifluoromethoxy)benzyl]-1H-indol-5-amine

Using 5-nitro-1-[3-(trifluoromethoxy)benzyl]-1H-indole (504 mg), 5% platinum-activated carbon (84 mg) and ethyl acetate (15 mL) and in the same manner as in Example G-8(ii), the title compound (466 mg) was obtained as a pale-yellow powder.

¹H-NMR (CDCl₃) δ: 3.50 (2H, br s), 5.26 (2H, s), 6.37 (1H, d, J=3.0 Hz), 6.62 (1H, dd, J=2.4 Hz, 8.7 Hz), 6.90-7.10 (5H, m), 7.08 (1H, d, J=8.7 Hz), 7.25-7.35 (1H, m).

### (iii) Production of tert-butyl{2-[4-({1-[3-(trifluoromethoxy)benzyl]-1H-indol-5-yl}amino)-5H-pyrrolo[3,2-d]pyrimidin-5-yl]ethyl}carbamate

Using tert-butyl[2-(4-chloro-5H-pyrrolo[3,2-d]pyrimidin-5-yl)ethyl]carbamate (405 mg), 1-[3-(trifluoromethoxy)benzyl]-1H-indol-5-amine (459 mg) and isopropyl alcohol (8.0 mL) and in the same manner as in Example G-8(iii), the title compound (692 mg) was obtained as a white amorphous.

¹H-NMR (CDCl₃) δ: 1.42 (9H, s), 3.45-3.55 (2H, m), 4.35-4.45 (2H, m), 4.93 (1H, br s), 5.33 (2H, s), 6.52 (1H, d, J=3.3 Hz), 6.55 (1H, d, J=3.0 Hz), 6.95-7.05 (2H, m), 7.10-7.25 (4H, m), 7.30 (1H, t, J=8.2 Hz), 7.45-7.55 (1H, m), 7.90 (1H, s), 8.18 (1H, br s), 8.44 (1H, s).

### (iv) Production of 5-(2-aminoethyl)-N-{1-[3-(trifluoromethoxy)benzyl]-1H-indol-5-yl}-5H-pyrrolo[3,2-d]pyrimidin-4-amine dihydrochloride

Using tert-butyl{2-[4-({1-[3-(trifluoromethoxy)benzyl]-1H-indol-5-yl}amino)-5H-pyrrolo[3,2-d]pyrimidin-5-yl]ethyl}carbamate (675 mg), 10% (W/W) hydrochloric acid/methanol (8.0 mL) and methanol (6.0 mL) and in the same manner as in Example G-8(iv), the title compound (568 mg) was obtained as an orange powder.

¹H-NMR (DMSO-d₆) δ: 3.20-3.35 (2H, m), 4.90-5.10 (2H, m), 5.54 (2H, s), 6.55 (1H, d, J=3.3 Hz), 6.68 (1H, d, J=3.0 Hz), 7.15-7.30 (4H, m), 7.46 (1H, t, J=7.8 Hz), 7.53 (1H, d, J=8.7 Hz), 7.63 (1H, d, J=3.3 Hz), 7.67 (1H, s), 8.01 (1H, d, J=3.0 Hz), 8.25-8.45 (3H, m), 8.55 (1H, s), 9.93 (1H, s).

### (v) Production of 3-hydroxy-3-methyl-N-{2-[4-({1-[3-(trifluoromethoxy)benzyl]-1H-indol-5-yl}amino)-5H-pyrrolo[3,2-d]pyrimidin-5-yl]ethyl}butanamide

Using 5-(2-aminoethyl)-N-{1-[3-(trifluoromethoxy)benzyl]-1H-indol-5-yl}-5H-pyrrolo[3,2-d]pyrimidin-4-amine dihydrochloride (200 mg), 3-hydroxy-3-methylbutanoic acid (62 mg), 1-hydroxybenzotriazole (76 mg), triethylamine (0.48 mL), 1-ethyl-3-(3-dimethylaminopropyl)carbodiimide hydrochloride (106 mg) and N,N-dimethylformamide (5.0 mL) and in the same manner as in Example G-8(v), the title compound (143 mg) was obtained as colorless crystals.

¹H-NMR (CDCl₃) δ: 1.26 (6H, s), 2.38 (2H, s), 3.55-3.70 (2H, m), 4.35-4.50 (2H, m), 5.32 (2H, s), 6.54 (2H, t, J=3.6 Hz), 6.80-6.90 (1H, m), 6.95-7.05 (2H, m), 7.10-7.20 (3H, m), 7.20-7.45 (3H, m), 7.88 (1H, s), 8.25 (1H, s), 8.43 (1H, s).

### Example G-10

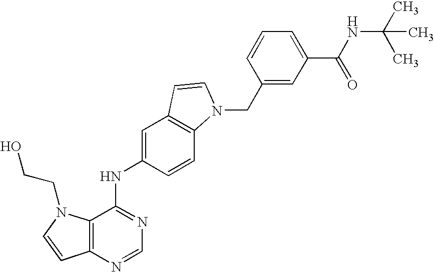

### Production of N-(tert-butyl)-3-[(5-{[5-(2-hydroxyethyl)-5H-pyrrolo[3,2-d]pyrimidin-4-yl]amino}-1H-indol-1-yl)methyl]benzamide

### (i) Production of methyl 3-[(5-nitro-1H-indol-1-yl)methyl]benzoate

Using 5-nitroindole (0.87 g), methyl 3-(bromomethyl)benzoate (1.35 g), potassium carbonate (0.89 g) and N,N-dimethylformamide (10 mL) and in the same manner as in Example G-8(i), the title compound (0.83 g) was obtained as pale-yellow crystals.

¹H-NMR (CDCl₃) δ: 3.89 (3H, s), 5.41 (2H, s), 6.75 (1H, d, J=3.3 Hz), 7.20-7.35 (3H, m), 7.39 (1H, t, J=7.8 Hz), 7.88 (1H, s), 7.97 (1H, d, J=7.8 Hz), 8.07 (1H, dd, J=2.4 Hz, 9.1 Hz), 8.60 (1H, d, J=1.8 Hz).

### (ii) Production of 3-[(5-nitro-1H-indol-1-yl)methyl]benzoic acid

To a suspension of methyl 3-[(5-nitro-1H-indol-1-yl)methyl]benzoate (0.75 g) in methanol (12 mL) was added 1N aqueous sodium hydroxide solution (12 mL) and the mixture was stirred at room temperature for 1.5 hr. Tetrahydrofuran (12 mL) was added to the mixture and the mixture was stirred at room temperature for 5 hr. Under ice-cooling, 1N hydrochloric acid (12 mL) was added to the reaction mixture, and the mixture was concentrated under reduced pressure. The precipitate was collected by filtration, and washed with ethanol and diisopropyl ether to give the title compound (621 mg) as a yellow powder.

¹H-NMR (DMSO-d₆) δ: 5.55 (2H, s), 6.80 (1H, d, J=2.4 Hz), 7.15-7.35 (2H, m), 7.67 (1H, d, J=9.0 Hz), 7.70-7.85 (3H, m), 7.99 (1H, d, J=7.8 Hz), 8.57 (1H, s).

### (iii) Production of N-(tert-butyl)-3-[(5-nitro-1H-indol-1-yl)methyl]benzamide

To a suspension of 3-[(5-nitro-1H-indol-1-yl)methyl]benzoic acid (600 mg) and tert-butylamine (222 mg) in N,N-dimethylformamide (5.0 mL) were added triethylamine (0.45 mL) and diethyl cyanophosphate (0.49 mL) under ice-cooling, and the mixture was stirred at room temperature for 21 hr. tert-Butylamine (0.32 mL) was added to the mixture, and the mixture was stirred at room temperature for 20 hr. Aqueous sodium bicarbonate was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:hexane=35:65→70:30) to give the title compound (266 mg) as a pale-yellow powder.

¹H-NMR (CDCl₃) δ: 1.45 (9H, s), 5.39 (2H, s), 5.87 (1H, br s), 6.70-6.75 (1H, m), 7.10 (1H, d, J=7.8 Hz), 7.25-7.30 (2H, m), 7.33 (1H, t, J=7.8 Hz), 7.55 (1H, d, J=7.8 Hz), 7.67 (1H, s), 8.06 (1H, dd, J=2.1 Hz, 9.0 Hz), 8.60 (1H, d, J=2.1 Hz).

### (iv) Production of 3-[(5-amino-1H-indol-1-yl)methyl]-N-(tert-butyl)benzamide

Using N-(tert-butyl)-3-[(5-nitro-1H-indol-1-yl)methyl]benzamide (263 mg), 5% platinum-activated carbon (44 mg) and ethyl acetate (10 mL) and in the same manner as in Example G-8(ii), the title compound (241 mg) was obtained as a pale-yellow amorphous.

¹H-NMR (CDCl₃) δ: 1.45 (9H, s), 3.50 (2H, br s), 5.28 (2H, s), 5.84 (1H, br s), 6.37 (1H, d, J=3.0 Hz), 6.62 (1H, dd, J=2.4 Hz, 8.7 Hz), 6.94 (1H, d, J=2.1 Hz), 7.00-7.15 (3H, m), 7.25-7.35 (1H, m), 7.54 (1H, d, J=7.5 Hz), 7.60 (1H, s).

### (v) Production of N-(tert-butyl)-3-[(5-{[5-(2-hydroxyethyl)-5H-pyrrolo[3,2-d]pyrimidin-4-yl]amino}-1H-indol-1-yl)methyl]benzamide

A mixture of 2-(4-chloro-5H-pyrrolo[3,2-d]pyrimidin-5-yl)ethyl benzoate (105 mg), 3-[(5-amino-1H-indol-1-yl)methyl]-N-(tert-butyl)benzamide (123 mg) and isopropyl alcohol (5.0 mL) was stirred at 80° C. for 16 hr. Under ice-cooling, aqueous sodium bicarbonate was added to the reaction mixture, and the mixture was extracted with ethyl acetate. The organic layer was concentrated under reduced pressure. The residue was separated and purified by silica gel column chromatography (eluent, ethyl acetate:methanol=100:0→95:5). To the obtained compound were added 1N aqueous sodium hydroxide solution (1.5 mL) and tetrahydrofuran (3.0 mL) and the mixture was stirred at room temperature for 18 hr. The reaction mixture was neutralized with 1N hydrochloric acid, and aqueous sodium bicarbonate and brine were added. The mixture was extracted with ethyl acetate, and the organic layer was dried over anhydrous magnesium sulfate and concentrated under reduced pressure. The residue was recrystallized from ethyl acetate/methanol to give the title compound (90 mg) as pale-yellow crystals.

¹H-NMR (CDCl₃) δ: 1.46 (9H, s), 4.00-4.10 (2H, m), 4.25-4.35 (2H, m), 5.33 (2H, s), 5.90 (1H, br s), 6.14 (1H, d, J=3.0 Hz), 6.52 (1H, d, J=2.7 Hz), 6.87 (1H, d, J=3.0 Hz), 7.10-7.35 (5H, m), 7.54 (1H, d, J=7.5 Hz), 7.63 (1H, s), 7.76 (1H, s), 8.19 (1H, s), 9.15 (1H, s).

### Example G-11

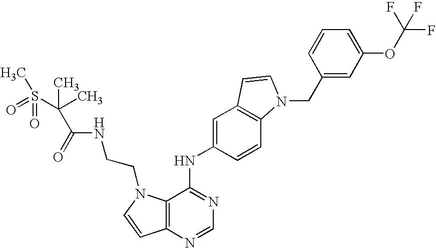

### Production of 2-methyl-2-(methylsulfonyl)-N-{2-[4-({1-[3-(trifluoromethoxy)benzyl]-1H-indol-5-yl}amino)-5H-pyrrolo[3,2-d]pyrimidin-5-yl]ethyl}propanamide

Using 5-(2-aminoethyl)-N-{1-[3-(trifluoromethoxy)benzyl]-1H-indol-5-yl}-5H-pyrrolo[3,2-d]pyrimidin-4-amine dihydrochloride (200 mg), 2-methyl-2-(methylsulfonyl)propanoic acid (87 mg), 1-hydroxybenzotriazole (76 mg), triethylamine (0.48 mL), 1-ethyl-3-(3-dimethylaminopropyl)carbodiimide hydrochloride (106 mg) and N,N-dimethylformamide (5.0 mL) and in the same manner as in Example G-8(v), the title compound (100 mg) was obtained as colorless crystals.

¹H-NMR (CDCl₃) δ: 1.61 (6H, s), 2.84 (3H, s), 3.65-3.75 (2H, m), 4.35-4.45 (2H, m), 5.34 (2H, s), 6.55 (1H, d, J=3.0 Hz), 6.60 (1H, d, J=3.0 Hz), 7.00-7.45 (9H, m), 7.86 (1H, s), 7.91 (1H, br s), 8.46 (1H, s).

### Example G-12

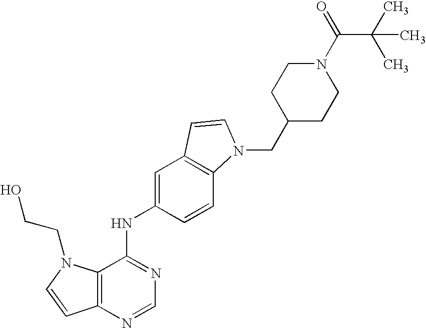

### Production of 2-{4-[(1-{[1-(2,2-dimethylpropanoyl)piperidin-4-yl]methyl}-1H-indol-5-yl)amino]-5H-pyrrolo[3,2-d]pyrimidin-5-yl}ethanol

### (i) Production of tert-butyl 4-[(5-nitro-1H-indol-1-yl)methyl]piperidine-1-carboxylate

To a solution of tert-butyl 4-(hydroxymethyl)piperidine-1-carboxylate (1.29 g) and triethylamine (1.17 mL) in tetrahydrofuran (30 mL) was added dropwise methanesulfonyl chloride (0.56 mL) under ice-cooling, and the mixture was stirred at room temperature for 1 hr. Aqueous sodium bicarbonate was added to the reaction mixture under ice-cooling, and the mixture was extracted with ethyl acetate. The organic layer was washed with brine, dried rover anhydrous magnesium sulfate and concentrated under reduced pressure to give a yellow oil. To a solution of 5-nitroindole (811 mg) in N,N-dimethylformamide (5.0 mL) was added sodium hydride (60% in oil (220 mg)) under ice-cooling, and the mixture was stirred at 0° C. for 10 min. Under ice-cooling, to the reaction mixture was added dropwise a solution of the yellow oil obtained above in N,N-dimethylformamide (5.0 mL) and the mixture was stirred at room temperature for 1 hr, and at 60° C. for 18 hr. Under ice-cooling, water was added to the reaction mixture, and the mixture was extracted with ethyl acetate. The organic layer was washed with brine, dried over anhydrous magnesium sulfate and concentrated under reduced pressure. The residue was separated and purified by silica gel column chromatography (eluent, ethyl acetate:hexane=30:70→50:50) to give the title compound (1.79 g) as a yellow amorphous form.

¹H-NMR (CDCl₃) δ: 1.15-1.30 (2H, m), 1.45 (9H, s), 1.50-1.60 (2H, m), 1.90-2.05 (1H, m), 2.55-2.70 (2H, m), 4.00-4.20 (4H, m), 6.69 (1H, d, J=3.0 Hz), 7.20 (1H, d, J=3.0 Hz), 7.34 (1H, d, J=9.0 Hz), 8.12 (1H, dd, J=2.1 Hz, 9.0 Hz), 8.60 (1H, d, J=2.1 Hz).

### (ii) Production of tert-butyl 4-[(5-amino-1H-indol-1-yl)methyl]piperidine-1-carboxylate

Using tert-butyl 4-[(5-nitro-1H-indol-1-yl)methyl]piperidine-1-carboxylate (0.90 g), 5% platinum-activated carbon (0.15 g) and ethyl acetate (10 mL) and in the same manner as in Example G-8(ii), the title compound (0.83 g) was obtained as a pale-red amorphous.

¹H-NMR (CDCl₃) δ: 1.10-1.25 (2H, m), 1.44 (9H, s), 1.50-1.60 (2H, m), 1.90-2.05 (1H, m), 2.55-2.70 (2H, m), 3.91 (2H, d, J=7.2 Hz), 4.00-4.20 (2H, m), 6.28 (1H, d, J=3.0 Hz), 6.67 (1H, dd, J=2.1 Hz, 8.7 Hz), 6.92 (1H, d, J=2.1 Hz), 6.96 (1H, d, J=3.3 Hz), 7.12 (1H, d, J=8.7 Hz).

### (iii) Production of tert-butyl 4-{[5-({5-[2-(benzoyloxy)ethyl]-5H-pyrrolo[3,2-d]pyrimidin-4-yl}amino)-1H-indol-1-yl]methyl}piperidine-1-carboxylate

A mixture of 2-(4-chloro-5H-pyrrolo[3,2-d]pyrimidin-5-yl)ethyl benzoate (686 mg), tert-butyl 4-[(5-amino-1H-indol-1-yl)methyl]piperidine-1-carboxylate (824 mg) and isopropyl alcohol (20 mL) was stirred at 80° C. for 12 hr. Under ice-cooling, aqueous sodium bicarbonate was added to the reaction mixture, and the mixture was extracted with ethyl acetate. The organic layer was washed with brine and concentrated under reduced pressure. The residue was separated and purified by silica gel column chromatography (eluent, ethyl acetate:methanol=100:0→395:5) to give the title compound (1.04 g) as a yellow powder.

¹H-NMR (CDCl₃) δ: 1.15-1.30 (2H, m), 1.45 (9H, s), 1.50-1.60 (2H, m), 1.95-2.10 (1H, m), 2.55-2.70 (2H, m), 3.98 (2H, d, J=7.5 Hz), 4.05-4.20 (2H, m), 4.55-4.70 (4H, m), 6.43 (1H, d, J=3.3 Hz), 6.63 (1H, d, J=3.3 Hz), 7.04 (1H, d, J=3.3 Hz), 7.25-7.35 (3H, m), 7.35-7.45 (3H, m), 7.50-7.65 (2H, m), 7.90-8.00 (2H, m), 8.48 (1H, s).

### (iv) Production of 2-(4-{[1-(piperidin-4-ylmethyl)-1H-indol-5-yl]amino}-5H-pyrrolo[3,2-d]pyrimidin-5-yl)ethyl benzoate dihydrochloride

To a solution of tert-butyl 4-{[5-({5-[2-(benzoyloxy)ethyl]-5H-pyrrolo[3,2-d]pyrimidin-4-yl}amino)-1H-indol-1-yl]methyl}piperidine-1-carboxylate (820 mg) in methanol (10 mL) was added 10% (W/W) hydrochloric acid/methanol (10 mL) and the mixture was stirred at room temperature for 17 hr. The reaction mixture was concentrated under reduced pressure, ethanol was added thereto, and the mixture was concentrated again. This operation was repeated twice. Diisopropyl ether was added, and the mixture was concentrated. The obtained powder was collected by filtration, and washed with diisopropyl ether to give the title compound (621 mg) as a pale-yellow powder.

¹H-NMR (DMSO-d₆) δ: 1.35-1.55 (2H, m), 1.60-1.70 (2H, m), 2.05-2.20 (1H, m), 2.70-2.90 (2H; m), 3.20-3.30 (2H, m), 4.14 (2H, d, J=6.9 Hz), 4.60-4.70 (2H, m), 5.10-5.20 (2H, m), 6.45 (1H, d, J=3.0 Hz), 6.65 (1H, d, J=2.7 Hz), 7.15 (1H, d, J=9.6 Hz), 7.40-7.50 (3H, m), 7.55 (1H, d, J=8.7 Hz), 7.66 (1H, t, J=7.5 Hz), 7.81 (2H, d, J=8.1 Hz), 8.07 (1H, d, J=3.0 Hz), 8.53 (1H, s), 8.55-8.70 (1H, br m), 8.90-9.00 (1H, br m), 9.90 (1H, br s).

### (v) Production of 2-{4-[(1-{[1-(2,2-dimethylpropanoyl)piperidin-4-yl]methyl}-1H-indol-5-yl)amino]-5H-pyrrolo[3,2-d]pyrimidin-5-yl}ethanol

To a suspension of 2-(4-{[1-(piperidin-4-ylmethyl)-1H-indol-5-yl]amino}-5H-pyrrolo[3,2-d]pyrimidin-5-yl)ethyl benzoate dihydrochloride (200 mg) in tetrahydrofuran (5.0 mL) were added dropwise triethylamine (0.23 mL) and 2,2-dimethylpropanoyl chloride (0.045 mL) under ice-cooling. The mixture was stirred at 0° C. for 30 min and at room temperature for 30 min. Under ice-cooling, water was added to the reaction mixture, and the mixture was extracted with ethyl acetate. The organic layer was washed with brine and concentrated under reduced pressure. The residue was separated and purified by silica gel column chromatography (eluent, ethyl acetate:methanol=100:0→95:5). To the obtained compound were added 1N aqueous sodium hydroxide solution (1.5 mL) and tetrahydrofuran (4.0 mL) and the mixture was stirred at room temperature for 15 hr. The reaction mixture was neutralized with 1N hydrochloric acid and aqueous sodium bicarbonate and brine were added. The mixture was extracted with ethyl acetate, and the organic layer was dried over anhydrous magnesium sulfate and concentrated under reduced pressure. The residue was recrystallized from ethyl acetate/methanol/diisopropyl ether to give the title compound (104 mg) as pale-yellow crystals.

¹H-NMR (CDCl₃) δ: 1.10-1.30 (2H, m), 1.27 (9H, s), 1.55-1.70 (2H, m), 2.05-2.20 (1H, m), 2.60-2.75 (2H, m), 3.98 (2H, d, J=7.2 Hz), 4.05-4.15 (2H, m), 4.30-4.50 (4H, m), 6.10 (1H, d, J=3.0 Hz), 6.46 (1H, d, J=3.0 Hz), 6.85 (1H, d, J=3.0 Hz), 7.04 (1H, d, J=3.0 Hz), 7.25-7.35 (1H, m), 7.35-7.45 (1H, m), 7.75 (1H, s), 8.18 (1H, s), 9.27 (1H, br s).

### Example G-13

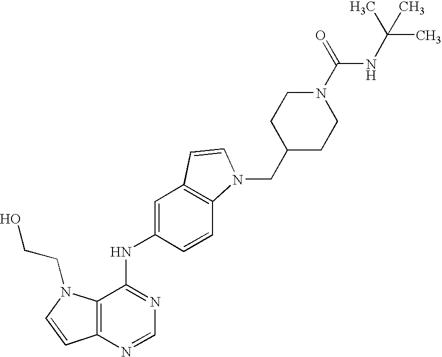

### Production of N-(tert-butyl)-4-[(5-{[5-(2-hydroxyethyl)-5H-pyrrolo[3,2-d]pyrimidin-4-yl]amino}-1H-indol-1-yl)methyl]piperidine-1-carboxamide

To a suspension of 2-(4-{[1-(piperidin-4-ylmethyl)-1H-indol-5-yl]amino}-5H-pyrrolo[3,2-d]pyrimidin-5-yl)ethyl benzoate dihydrochloride (200 mg) in tetrahydrofuran (5.0 mL) were added dropwise triethylamine (0.23 mL) and 2-isocyanate-2-methylpropane (0.045 mL) under ice-cooling. The reaction mixture was stirred at room temperature for 1.5 hr, water was added to the reaction mixture under ice-cooling, and the mixture was extracted with ethyl acetate. The organic layer was washed with brine and concentrated under reduced pressure. The residue was separated and purified by silica gel column chromatography (eluent, ethyl acetate:methanol=100:0→95:5). To the obtained compound were added 1N aqueous sodium hydroxide solution (1.6 mL) and tetrahydrofuran (4.0 mL) and the mixture was stirred at room temperature for 15 hr. The reaction mixture was neutralized with 1N hydrochloric acid and aqueous sodium bicarbonate and brine were added. The mixture was extracted with ethyl acetate and the organic layer was dried over anhydrous magnesium sulfate and concentrated under reduced pressure. The residue was recrystallized from ethyl acetate/methanol/diisopropyl ether to give the title compound (112 mg) as pale-yellow crystals.

¹H-NMR (CDCl₃) δ: 1.15-1.30 (2H, m), 1.34 (9H, s), 1.50-1.65 (2H, m), 1.95-2.10 (1H, m), 2.55-2.70 (2H, m), 3.85-3.95 (2H, m), 3.99 (2H, d, J=7.2 Hz), 4.05-4.15 (2H, m), 4.29 (1H, s), 4.30-4.40 (2H, m), 6.19 (1H, d, J=3.0 Hz), 6.45-6.50 (1H, m), 6.90-6.95 (1H, m), 7.05-7.10 (1H, m), 7.25-7.45 (2H, m), 7.75 (1H, s), 8.23 (1H, s), 9.20 (1H, br s).

### Example G-14

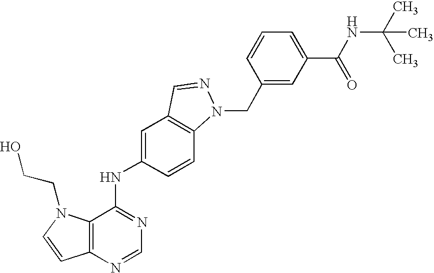

### Production of N-(tert-butyl)-3-[(5-{[5-(2-hydroxyethyl)-5H-pyrrolo[3,2-d]pyrimidin-4-yl]amino}-1H-indazol-1-yl)methyl]benzamide

### (i) Production of methyl 3-[(5-nitro-1H-indazol-1-yl)methyl]benzoate

To a solution of 5-nitroindazole (816 mg) and methyl 3-(bromomethyl)benzoate (2.29 g) in N,N-dimethylformamide (10 mL) was added potassium carbonate (2.07 g) under ice-cooling, and the mixture was stirred at room temperature for 2 hr. Under ice-cooling, water was added to the reaction mixture, and the mixture was extracted with ethyl acetate. The organic layer was washed with brine, dried over anhydrous magnesium sulfate and concentrated under reduced pressure. The residue was separated and purified by silica gel column chromatography (eluent, ethyl acetate:hexane=20:80→50:50) to give the title compound (805 mg) as pale-yellow crystals.

¹H-NMR (CDCl₃) δ: 3.90 (3H, s), 5.68 (2H, s), 7.30-7.45 (3H, m), 7.90-8.00 (2H, m), 8.23 (1H, dd, J=1.8 Hz, 9.0 Hz), 8.20-8.25 (1H, m), 8.74 (1H, d, J=1.8 Hz).

### (ii) Production of 3-[(5-nitro-1H-indazol-1-yl)methyl]benzoic acid

Using methyl 3-[(5-nitro-1H-indazol-1-yl)methyl]benzoate (0.79 g), 1N aqueous sodium hydroxide solution (12 mL), methanol (15 mL) and tetrahydrofuran (15 mL) and in the same manner as in Example G-10(ii), the title compound (732 mg) was obtained as a pale-yellow powder.

¹H-NMR (DMSO-d₆) δ: 5.86 (2H, s), 7.40-7.55 (2H, m), 7.75-7.90 (2H, m), 8.00 (1H, d, J=9.3 Hz), 8.25 (1H, dd, J=2.1 Hz, 9.3 Hz), 8.48 (1H, s), 8.86 (1H, d, J=2.1 Hz), 13.07 (1H, br s).

### (iii) Production of N-(tert-butyl)-3-[(5-nitro-1H-indazol-1-yl)methyl]benzamide

To a suspension of 3-[(5-nitro-1H-indazol-1-yl)methyl]benzoic acid (595 mg) in tetrahydrofuran (10 mL) were added N,N-dimethylformamide (one drop) and thionyl chloride (0.144 mL) and the mixture was stirred at room temperature for 3.5 hr. To a solution of tert-butylamine (0.73 g) and triethylamine (0.25 g) in N,N-dimethylformamide (4.0 mL) was added the above-mentioned reaction mixture under ice-cooling, and the mixture was stirred at room temperature for 4.5 hr. Water was added to the reaction mixture and the mixture was extracted with ethyl acetate. The organic layer was washed with brine and dried over anhydrous magnesium sulfate. After concentration under reduced pressure, the residue was separated and purified by silica gel column chromatography (eluent, ethyl acetate:hexane=30:70→50:50) to give the title compound (0.56 g) as a pale-yellow amorphous form.

¹H-NMR (CDCl₃) δ: 1.45 (9H, s), 5.66 (2H, s), 5.88 (1H, br s), 7.20-7.30 (1H, m), 7.35 (1H, t, J=7.5 Hz), 7.40 (1H, d, J=9.0 Hz), 7.56 (1H, d, J=7.5 Hz), 7.73 (1H, s), 8.23 (1H, dd, J=2.1 Hz, 9.0 Hz), 8.25 (1H, s), 8.74 (1H, d, J=2.1 Hz).

### (iv) Production of 3-[(5-amino-1H-indazol-1-yl)methyl]-N-(tert-butyl)benzamide

Using N-(tert-butyl)-3-[(5-nitro-1H-indazol-1-yl)methyl]benzamide (0.55 g), 5% platinum-activated carbon (92 mg) and ethyl acetate (20 mL) and in the same manner as in Example G-8(ii), the title compound (0.47 g) was obtained as a white powder.

¹H-NMR (CDCl₃) δ: 1.44 (9H, s), 3.61 (2H, br s), 5.55 (2H, s), 5.86 (1H, br s), 6.81 (1H, d, J=9.0 Hz), 6.95-7.00 (1H, m), 7.14 (1H, d, J=8.7 Hz), 7.20 (1H, d, J=7.5 Hz), 7.30 (1H, t, J=7.5 Hz), 7.56 (1H, d, J=8.1 Hz), 7.63 (1H, s), 7.83 (1H, s).

### (v) Production of N-(tert-butyl)-3-[(5-{[5-(2-hydroxyethyl)-5H-pyrrolo[3,2-d]pyrimidin-4-yl]amino}-1H-indazol-1-yl)methyl]benzamide

Using 2-(4-chloro-5H-pyrrolo[3,2-d]pyrimidin-5-yl)ethyl benzoate (151 mg), 3-[(5amino-1H-indazol-1-yl)methyl]-N-(tert-butyl)benzamide (177 mg), isopropyl alcohol (8.0 mL), 1N aqueous sodium hydroxide solution (2.0 mL) and tetrahydrofuran (4.0 mL) and in the same manner as in Example G-10(v), the title compound (169 mg) was obtained as colorless crystals.

¹H-NMR (CDCl₃) δ: 1.45 (9H, s), 4.05-4.15 (2H, m), 4.30-4.40 (2H, m), 5.59(2H, s), 5.92 (1H, br s), 6.18 (1H, d, J=3.0 Hz), 6.93 (1H, d, J=3.6 Hz), 7.25-7.35 (4H, m), 7.40-7.50 (1H, m), 7.56 (1H, d, J=7.2 Hz), 7.65 (1H, s), 7.88 (1H, s), 7.98 (1H, s), 8.22 (1H, s), 9.33 (1H, br s).

### Example G-15

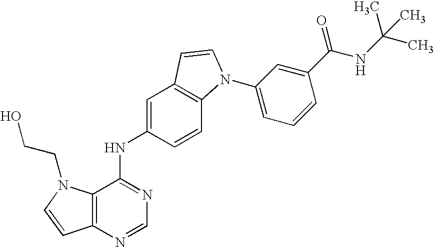

### Production of N-(tert-butyl)-3-(5-{[5-(2-hydroxyethyl)-5H-pyrrolo[3,2-d]pyrimidin-4-yl]amino}-1H-indol-1-yl)benzamide

### (i) Production of ethyl 3-(5-nitro-1H-indol-1-yl)benzoate

A mixed solution of 5-nitroindole (1.62 g), ethyl 3-iodobenzoate (3.04 g), N,N-dimethylenediamine (0.97 g), potassium carbonate (1.66 g), copper(I) iodide (0.19 g) and toluene (10 mL) was stirred at 120° C. for 24 hr. Under ice-cooling, water was added to the reaction mixture, and the mixture was extracted with ethyl acetate. The organic layer was washed with brine, dried over anhydrous magnesium sulfate and concentrated under reduced pressure: The residue was separated and purified by silica gel column chromatography (eluent, ethyl acetate:hexane=20:80→40:60) to give the title compound (0.95 g) a pale-yellow powder.

¹H-NMR (CDCl₃) δ: 1.43 (3H, t, J=7.0 Hz), 4.44 (2H, q, J=7.0 Hz), 6.89 (1H, d, J=3.3 Hz), 7.50-7.55 (2H, m), 7.60-7.75 (2H, m), 8.10-8.20 (3H, m), 8.66 (1H, d, J=2.4 Hz).

### (ii) Production of 3-(5-nitro-1H-indol-1-yl)benzoic acid

Using ethyl 3-(5-nitro-1H-indol-1-yl)benzoate (0.95 g), 1N aqueous sodium hydroxide solution (15 mL), ethanol (15 mL) and tetrahydrofuran (15 mL) and in the same manner as in Example G-10(ii), the title compound (0.71 g) was obtained as a yellow powder.

¹H-NMR (DMSO-d₆) δ: 6.95-7.05 (1H, m), 7.60-7.75 (2H, m), 7.83 (1H, d, J=7.2 Hz), 7.90-8.10 (4H, m), 8.68 (1H, s).

### (iii) Production of N-(tert-butyl)-3-(5-nitro-1H-indol-1-yl)benzamide

Using 3-(5-nitro-1H-indol-1-yl)benzoic acid (565 mg), thionyl chloride (0.144 mL), tetrahydrofuran (10 mL), tert-butylamine (0.73 g), triethylamine (0.25 g) and N,N-dimethylformamide (4.0 mL) and in the same manner as in Example G-14(iii), the title compound (0.32 g) was obtained as a yellow powder.

¹H-NMR (CDCl₃) δ: 1.50 (9H, s), 5.99 (1H, br s), 6.89 (1H, d, J=3.3 Hz), 7.50-7.65 (4H, m), 7.65-7.75 (1H, m), 7.93 (1H, s), 8.13 (1H, dd, J=2.1 Hz, 9.0 Hz), 8.66 (1H, d, J=2.4 Hz).

### (iv) Production of 3-(5-amino-1H-indol-1-yl)-N-(tert-butyl)benzamide

Using N-(tert-butyl)-3-(5-nitro-1H-indol-1-yl)benzamide (0.32 g), 5% platinum-activated carbon (0.05 g) and ethyl acetate (20 mL) and in the same manner as in Example G-8(ii), the title compound (253 mg) was obtained as a pale-pink amorphous.

¹H-NMR (CDCl₃) δ: 1.49 (9H, s), 3.58 (2H, br s), 5.94 (1H, br s), 6.51 (1H, d, J=3.0 Hz), 6.68 (1H, dd, J=2.1 Hz, 8.7 Hz), 6.96 (1H, d, J=2.1 Hz), 7.20-7.30 (1H, m), 7.37 (1H, d, J=8.7 Hz), 7.50-7.65 (3H, m), 7.84 (1H, s).

### (v) Production of N-(tert-butyl)-3-(5-{[5-(2-hydroxyethyl)-5H-pyrrolo[3,2-d]pyrimidin-4-yl]amino}-1H-indol-1-yl)benzamide

Using 2-(4-chloro-5H-pyrrolo[3,2-d]pyrimidin-5-yl)ethyl benzoate (164 mg), 3-(5-amino-1H-indol-1-yl)-N-(tert-butyl)benzamide (200 mg), isopropyl alcohol (8.0 mL), 1N aqueous sodium hydroxide solution (2.5 mL) and tetrahydrofuran (5.0 mL) and in the same manner as in Example G-10(v), the title compound (154 mg) was obtained as pale-yellow crystals.

¹H-NMR (CDCl₃) δ: 1.51 (9H, s), 4.10-4.15 (2H, m), 4.35-4.45 (2H, m), 6.03 (1H, br s), 6.17 (1H, d, J=3.3. Hz), 6.67 (1H, d, J=3.3 Hz), 6.91 (1H, d, J=3.3 Hz), 7.30-7.40 (2H, m), 7.50-7.70 (4H, m), 7.80-7.85 (1H, m), 7.89 (1H, s), 8.23 (1H, s), 9.24 (1H, br s).

### Example G-16

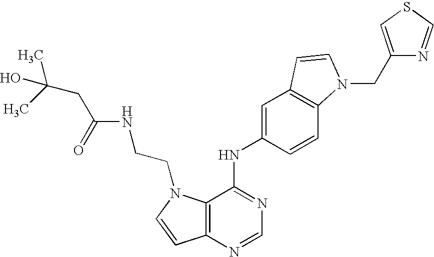

### Production of 3-hydroxy-3-methyl-N-[2-(4-{[1-(1,3-thiazol-4-ylmethyl)-1H-indol-5-yl]amino}-5H-pyrrolo[3,2-d]pyrimidin-5-yl)ethyl]butanamide

### (i) Production of 5-nitro-1-(1,3-thiazol-4-ylmethyl)-1H-indole

Using 5-nitroindole (1.62 g), 4-(chloromethyl)-1,3-thiazole hydrochloride (1.87 g), potassium carbonate, (4.15 g) and N,N-dimethylformamide (20 mL) and in the same manner as in Example G-8(i), the title compound (1.98 g) was obtained as a yellow powder.

¹H-NMR (CDCl₃) δ: 5.54 (2H, s), 6.74 (1H, d, J=3.3 Hz), 6.95-7.00 (1H, m), 7.30-7.45 (2H, m), 8.10 (1H, dd, J=2.1 Hz, 9.0 Hz), 8.60 (1H, d, J=2.1 Hz), 8.81 (1H, d, J=2.1 Hz).

### (ii) Production of 1-(1,3-thiazol-4-ylmethyl)-1H-indol-5-amine

To a solution of 5-nitro-1-(1,3-thiazol-4-ylmethyl)-1H-indole (519 mg) in ethyl acetate (20 mL)/methanol (4 mL) was added 5% platinum-activated carbon (87 mg) under a nitrogen atmosphere. The reaction mixture was stirred at room temperature for 3 days under a hydrogen atmosphere. Under a nitrogen atmosphere, 10% palladium/carbon (87 mg) was added, and the mixture was stirred at room temperature for 9 hr under a hydrogen atmosphere. The catalyst was filtered off, and the filtrate was concentrated under reduced pressure. The residue was separated and purified by silica gel column chromatography (eluent, ethyl acetate:hexane=60:40→100:0) to give the title compound (424 mg) as a red oil.

¹H-NMR (CDCl₃) δ: 3.50 (2H, br s), 5.44 (2H, s), 6.37 (1H, d, J=3.3 Hz), 6.64 (1H, dd, J=2.1 Hz, 8.4 Hz), 6.75-6.80 (1H, m), 6.94 (1H, d, J=2.1 Hz), 7.10-7.15 (2H, m), 8.77 (1H, d, J=1.8 Hz).

### (iii) Production of tert-butyl[2-(4-{[1-(1,3-thiazol-4-ylmethyl)-1H-indol-5-yl]amino}-5H-pyrrolo[3,2-d]pyrimidin-5-yl)ethyl]carbamate

Using tert-butyl[2-(4-chloro-5H-pyrrolo[3,2-d]pyrimidin-5-yl)ethyl]carbamate (446 mg), 1-(1,3-thiazol-4-ylmethyl)-1H-indol-5-amine (414 mg) and isopropyl alcohol (10 mL) and in the same manner as in Example G-8(iii), the title compound (697 mg) was obtained as a pale-red amorphous.

¹H-NMR (CDCl₃) δ: 1.43 (9H, s), 3.45-3.55 (2H, m), 4.35-4.45 (2H, m), 4.92 (1H, br s), 5.51 (2H, s), 6.51 (1H, d, J=3.0 Hz), 6.56 (1H, d, J=3.0 Hz), 6.81 (1H, s), 7.13 (1H, d, J=3.3 Hz), 7.20 (1H, d, J=3.0 Hz), 7.28 (1H, d, J=8.7 Hz), 7.48 (1H, d, J=7.2′ Hz), 7.88 (1H, s), 8.18 (1H, br s), 8.44 (1H, s), 8.79 (1H, d, J=2.1 Hz).

### (iv) Production of 5-(2-aminoethyl)-N-[1-(1,3-thiazol-4-ylmethyl)-1H-indol-5-yl]-5H-pyrrolo[3,2-d]pyrimidin-4-amine dihydrochloride

Using tert-butyl[2-(4-{[1-(1,3-thiazol-4-ylmethyl)-1H-indol-5-yl]amino}-5H-pyrrolo[3,2-d]pyrimidin-5-yl)ethyl]carbamate (675 mg), 10% (W/W) hydrochloric acid/methanol (10 mL) and methanol (8.0 mL) and in the same manner as in Example G-8(iv), the title compound (610 mg) was obtained as a pale-green powder.

¹H-NMR (DMSO-d₆) δ: 3.25-3.35 (2H, m), 4.90-5.10 (2H, m), 5.57 (2H, s), 6.50 (1H, d, J=3.3 Hz), 6.69 (1H, d, J=3.0 Hz), 7.20 (1H, dd, J=1.8 Hz, 8.4 Hz), 7.50-7.65 (4H, m), 8.01 (1H, d, J=3.0 Hz), 8.25-8.45 (3H, m), 8.55 (1H, s), 9.05 (1H, d, J=1.8 Hz), 9.97 (1H, s).

### (v) Production of 3-hydroxy-3-methyl-N-[2-(4-{[1-(1,3-thiazol-4-ylmethyl)-1H-indol-5-yl]amino}-5H-pyrrolo[3,2-d]pyrimidin-5-yl)ethyl]butanamide

Using 5-(2-aminoethyl)-N-[1-(1,3-thiazol-4-ylmethyl)-1H-indol-5-yl]-5H-pyrrolo[3,2-d]pyrimidin-4-amine dihydrochloride (200 mg), 3-hydroxy-3-methylbutanoic acid (71 mg), 1-hydroxybenzotriazole (88 mg), triethylamine (0.56 mL), 1-ethyl-3-(3-dimethylaminopropyl)carbodiimide hydrochloride (123 mg) and N,N-dimethylformamide (5.0′mL) and in the same manner as in Example G-8(v), the title compound (118 mg) was obtained as colorless crystals.

¹H-NMR (CDCl₃) δ: 1.27 (6H, s), 2.38 (2H, s), 3.55-3.70 (2H, m), 4.35-4.60 (2H, m), 5.50 (2H, s), 6.50-6.60 (2H, m), 6.65-6.75 (1H, m), 6.82 (1H, s), 7.13 (1H, d, J=3.3 Hz), 7.15-7.45 (4H, m), 7.83 (1H, s), 8.25 (1H, s), 8.42 (1H, s), 8.78 (1H, d, J=1.5 Hz).

### Example G-17

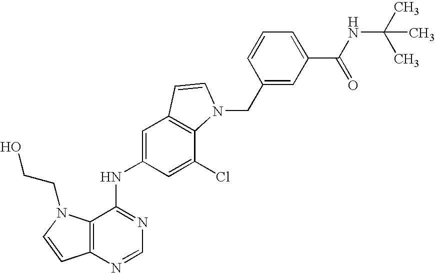

### Production of N-(tert-butyl)-3-[(7-chloro-5-{[5-(2-hydroxyethyl)-5H-pyrrolo[3,2-d]pyrimidin-4-yl]amino}-1H-indol-1-yl)methyl]benzamide

### (i) Production of 7-chloro-5-nitroindoline

A mixed solution of 5-nitroindoline (3.28 g), N-chlorosuccimide (2.94 g) and acetonitrile (100 mL) was refluxed for 19 hr. Water was added to the reaction mixture, and the mixture was extracted with ethyl acetate. The organic layer was washed with brine, dried over anhydrous magnesium sulfate and concentrated under reduced pressure. The residue was separated and purified by silica gel column chromatography (eluent, ethyl acetate:hexane=30:70→50:50) to give the title compound (2.09 g) as a yellow powder.

¹H-NMR (CDCl₃) δ: 3.23 (2H, t, J=8.7 Hz), 3.84 (2H, t, J=8.7 Hz), 4.68 (1H, br s), 7.85-7.90 (1H, m), 8.05-8.10 (1H, m).

### (ii) Production of 7-chloro-5-nitro-1H-indole

To a solution of 7-chloro-5-nitroindoline (2.09 g) in ethyl acetate (100 mL) was added manganese oxide (9.13 g), and the mixture was stirred at 60° C. for 15 hr. Manganese oxide was filtered off, and the filtrate was concentrated under reduced, pressure. The residue was separated and purified by silica gel column chromatography (eluent, ethyl acetate) and washed with hexane to give the title compound (1.92 g) as a pale-brown powder.

¹H-NMR (CDCl₃) δ: 6.79-6.82 (1H, m), 7.40-7.45 (1H, m), 8.15 (1H, d, J=1.8 Hz), 8.53 (1H, d, J=1.8 Hz), 8.65-8.85 (1H, m).

### (iii) Production of methyl 3-[(7-chloro-5-nitro-1H-indol-1-yl)methyl]benzoate

Using 7-chloro-5-nitro-1H-indole (950 mg), methyl 3-(bromomethyl)benzoate (1.22 g), potassium carbonate (0.80 g) and N,N-dimethylformamide (15 mL) and in the same manner as in Example G-8(i), the title compound (1.43 g) was obtained as pale-yellow crystals.

¹H-NMR (CDCl₃) δ: 3.89 (3H, s), 5.84 (2H, s), 6.80 (1H, d, J=3.3 Hz), 7.13 (1H, dd, J=0.9 Hz, 7.8 Hz), 7.25-7.30 (1H, m), 7.38 (1H, t, J=7.8 Hz), 7.79 (1H, s), 7.95 (1H, d, J=8.1 Hz), 8.07 (1H, d, J=2.1 Hz), 8.50 (1H, d, J=2.1 Hz).

### (iv) Production of 3-[(7-chloro-5-nitro-1H-indol-1-yl)methyl]benzoic acid

Using methyl 3-[(7-chloro-5-nitro-1H-indol-1-yl)methyl]benzoate (1.30 g), 1N aqueous sodium hydroxide solution (20 mL), methanol (20 mL) and tetrahydrofuran (30 mL) and in the same manner as in Example G-10(ii), the title compound (1.20 g) was obtained as a pale-yellow powder.

¹H-NMR (DMSO-d₆) δ: 5.92 (2H, s), 6.99 (1H, d, J=3.0 Hz), 7.26 (1H, d, J=7.5 Hz), 7.44 (1H, t, J=7.5 Hz), 7.56 (1H, s), 7.82 (1H, d, J=7.5 Hz), 7.88 (1H, d, J=3.0 Hz), 7.98 (1H, d, J=1.8 Hz), 8.62 (1H, d, J=1.8 Hz), 12.90-13.10 (1H, br).

### (v) Production of N-(tert-butyl)-3-[(7-chloro-5-nitro-1H-indol-1-yl)methyl]benzamide

Using 3-[(7-chloro-5-nitro-1H-indol-1-yl)methyl]benzoic acid (595 mg), thionyl chloride (0.13 mL), tetrahydrofuran (10 mL), tert-butylamine (0.66 g), triethylamine (0.23 g) and N,N-dimethylformamide (4.0 mL) and in the same manner as in Example G-14(iii), the title compound (460 mg) was obtained as pale-yellow crystals.

¹H-NMR (CDCl₃) δ: 1.45 (9H, s), 5.83 (2H, s), 5.86 (1H, br s), 6.79 (1H, t, J=3.3 Hz), 7.02 (1H, d, J=7.8 Hz), 7.20-7.30 (1H, m), 7.33 (1H, t, J=7.8 Hz), 7.53 (1H, d, J=7.8 Hz), 7.58 (1H, s), 8.07 (1H, d, J=2.4 Hz), 8.49 (1H, d, J=2.4 Hz).

### (vi) Production of 3-[(5-amino-7-chloro-1H-indol-1-yl)methyl]-N-(tert-butyl)benzamide

N-(tert-Butyl)-3-[(7-chloro-5-nitro-1H-indol-1-yl)methyl]benzamide (212 mg) was suspended in ethanol (10 mL)/water (1 mL), calcium chloride (30.5 mg) was added, and the mixture was dissolved at 90° C. Reduced iron (184 mg) was added to the mixture, and the mixture was stirred at 90° C. for 4 hr. The reaction mixture was allowed to return to room temperature, and the insoluble material was filtered off. The filtrate was concentrated under reduced pressure, ethyl acetate was added thereto, and the mixture was washed with brine. The mixture was dried over anhydrous magnesium sulfate and concentrated under reduced pressure to give the title compound (203 mg) as a yellow amorphous form.

¹H-NMR (CDCl₃) δ: 1.44 (9H, s), 3.45-3.60 (2H, br), 5.69 (2H, s), 5.84 (1H, br s), 6.37 (1H, d, J=3.3 Hz), 6.61 (1H, d, J=2.0 Hz), 6.82 (1H, d, J=2.0 Hz), 6.95-7.05 (1H, m), 7.02 (1H, d, J=3.3 Hz), 7.29 (1H, t, J=7.8 Hz), 7.50-7.55 (1H, m), 7.53 (1H, d, J=7.8 Hz).

### (vii) Production of N-(tert-butyl)-3-[(7-chloro-5-{[5-(2-hydroxyethyl)-5H-pyrrolo[3,2-d]pyrimidin-4-yl]amino}-1H-indol-1-yl)methyl]benzamide

Using 2-(4-chloro-5H-pyrrolo[3,2-d]pyrimidin-5-yl)ethyl benzoate (151 mg), 3-[(5-amino-7-chloro-1H-indol-1-yl)methyl]-N-(tert-butyl)benzamide (196 mg), isopropyl alcohol (8.0 mL), 1N aqueous sodium hydroxide solution (2.0 mL) and tetrahydrofuran (4.0 mL) and in the same manner as in Example G-10(v), the title compound (157 mg) was obtained as colorless crystals.

¹H-NMR (CDCl₃) δ: 1.45 (9H, s), 4.05-4.15 (2H, m), 4.30-4.40 (2H, m), 5.75 (2H, s), 5.89 (1H, s), 6.17 (1H, d, J=3.0 Hz), 6.53 (1H, d, J=3.0 Hz), 6.92 (1H, d, J=3.0 Hz), 7.05 (1H, d, J=7.2 Hz), 7.09 (1H, d, J=3.0 Hz), 7.25-7.35 (2H, m), 7.50-7.55 (2H, m), 7.65-7.70 (1H, m), 8.22 (1H, s), 9.22 (1H, s).

### Example G-18

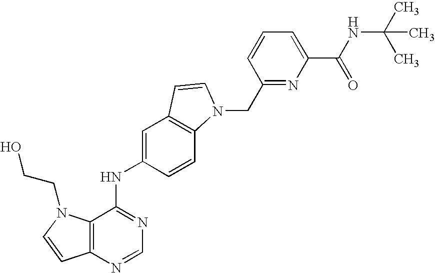

### Production of N-(tert-butyl)-6-[(5-{[5-(2-hydroxyethyl)-5H-pyrrolo[3,2-d]pyrimidin-4-yl]amino}-1H-indol-1-yl)methyl]pyridine-2-carboxamide

### (i) Production of ethyl 6-(hydroxymethyl)pyridine-2-carboxylate

To a solution of diethyl pyridine-2,6-dicarboxylate (4.46 g) in ethanol (50 mL) was added sodium borohydride (454 mg) and the mixture was stirred at room temperature for 3 hr. The reaction mixture was concentrated under reduced pressure, brine was added thereto, and the mixture was extracted with ethyl acetate. The aqueous layer was salted out, and the mixture was extracted with ethyl acetate. The combined organic layers were dried) over anhydrous magnesium sulfate, and concentrated under reduced pressure. The residue was separated and purified by silica gel column chromatography (eluent, ethyl acetate:hexane=50:50→100:0) to give the title compound (2.42 g) as colorless crystals.

¹H-NMR (CDCl₃) δ: 1.44 (3H, t, J=7.2 Hz), 3.53 (1H, t, J=5.4 Hz), 4.46 (2H, q, J=7.2 Hz), 4.86 (2H, d, J=5.4 Hz), 7.49 (1H, d, J=7.8 Hz), 7.84 (1H, t, J=7.8 Hz), 8.02 (1H, d, J=7.8 Hz).

### (ii) Production of ethyl 6-[(5-nitro-1H-indol-1-yl)methyl]pyridine-2-carboxylate

To a solution of ethyl 6-(hydroxymethyl)pyridine-2-carboxylate (1.00 g) and triethylamine (0.84 mL) in tetrahydrofuran (20 mL) was added dropwise methanesulfonyl chloride (0.43 mL) under ice-cooling, and the mixture was stirred at room temperature for 1.5 hr. Under ice-cooling, to the reaction mixture were added aqueous sodium bicarbonate and brine, and the mixture was extracted with ethyl acetate. The organic layer was dried over anhydrous magnesium sulfate and concentrated under reduced pressure. To the obtained compound were added 5-nitroindole (757 mg), potassium carbonate (0.97 g) and N,N-dimethylformamide (10 mL) and the mixture was stirred at room temperature for 24 hr at 60° C. for 20 hr, and at 80° C. for 1 hr. Under ice-cooling, water was added to the reaction mixture, and the mixture was extracted with ethyl acetate. The aqueous layer was salted out, and the mixture was extracted with ethyl acetate. The combined organic layers were dried over anhydrous magnesium sulfate concentrated under reduced pressure, and the residue was recrystallized from ethyl acetate to give the title compound (1.31 g) as a pale-yellow powder.

¹H-NMR (CDCl₃) δ: 1.47 (3H, t, J=7.2 Hz), 4.51 (2H, q, J=7.2 Hz), 5.62 (2H, s), 6.76 (1H, d, J=7.8 Hz), 6.79 d, J=3.0 Hz), 7.30 (1H, d, J=9.0 Hz), 7.36 (1H, d, J=3.0 Hz), 7.70 (1H, t, J=7.8 Hz), 8.02 (1H, d, J=7.8 Hz), 8.08 (1g, dd, J=2.1 Hz, 9.0 H), 8.63 (1H, d, J=2.1 Hz).

### (iii) Production of 6-[(5-nitro-1H-indol-1-yl)methyl]pyridine-2-carboxylic acid

Using ethyl 6-[(5-nitro-1H-indol-1-yl)methyl]pyridine-2-carboxylate (1.20 g), 1N aqueous sodium hydroxide solution (20 mL), ethanol (20 mL) and tetrahydrofuran (40 mL) and in the same manner as in Example G-10(ii), the title compound (1.01 g) was obtained as a pale-yellow powder.

¹H-NMR (DMSO-d₆) δ: 5.68 (2H, s), 6.80-6.85 (1H, m), 6.89 (1H, d, J=7.2 Hz), 7.67 (1H, d, J=8.1 Hz), 7.70-7.90 (3H, m), 7.99 (1H, d, J=9.0 Hz), 8.60 (1H, s).

### (iv) Production of N-(tert-butyl)-6-[(5-nitro-1H-indol-1-yl)methyl]pyridine-2-carboxamide

Using 6-[(5-nitro-1H-indol-1-yl)methyl]pyridine-2-carboxylic acid (0.95 g), thionyl chloride (0.23 mL), tetrahydrofuran (20 mL), tert-butylamine (1.17 g), triethylamine (0.40 g) and N,N-dimethylformamide (8.0 mL) and in the same manner as in Example G-14(iii), the title compound (636 mg) was obtained as a pale-yellow amorphous.

¹H-NMR (CDCl₃) δ: 1.41 (9H, s), 5.51 (2H, s), 6.78 (1H, d, J=3.3 Hz), 6.98 (1H, d, J=7.8 Hz), 7.30-7.40 (2H, m), 7.66 (1H, br s), 7.76 (1H, t, J=7.8 Hz), 8.05-8.15 (2H, m), 8.63 (1H, d, J=2.1 Hz).

### (v) Production of 6-[(5-amino-1H-indol-1-yl)methyl]-N-(tert-butyl)pyridine-2-carboxamide

To a solution of N-(tert-butyl)-6-[(5-nitro-1H-indol-1-yl)methyl]pyridine-2-carboxamide (211 mg) in ethyl acetate (8.0 mL) was added 5% platinum-activated carbon (35 mg) under a nitrogen atmosphere. The reaction mixture was stirred under a hydrogen atmosphere at room temperature for 5 hr. Under a nitrogen atmosphere, 10% palladium/carbon (69 mg) was added to the reaction mixture, and the mixture was further stirred under a hydrogen atmosphere at room temperature for 2.5 hr. The catalyst was filtered off, and the filtrate was concentrated under reduced pressure. The residue was separated and purified by silica gel column chromatography (eluent, ethyl acetate:hexane=50:50→80:20) to give the title compound (160 mg) as a pale-pink amorphous.

¹H-NMR (CDCl₃) δ: 1.48 (9H, s), 3.52 (2H, br s), 5.39 (2H, s), 6.41 (1H, d, J=3.0 Hz); 6.64 (1H, dd, J=2.4 Hz, 8.7 Hz), 6.82 (1H, d, J=8.1 Hz), 6.97 (1H, d, J=2.4 Hz), 7.00-7.10 (1H, m), 7.11 (1H, d, J=3.0 Hz), 7.68 (1H, t, J=7.5 Hz), 7.89 (1H, br s), 8.03 (1H, d, J=7.5 Hz).

### (vi) Production of N-(tert-butyl)-6-[(5-{[5-(2-hydroxyethyl)-5H-pyrrolo[3,2-d]pyrimidin-4-yl]amino}-1H-indol-1-yl)methyl]pyridine-2-carboxamide

Using 2-(4-chloro-5H-pyrrolo[3,2-d]pyrimidin-5-yl)ethyl benzoate (136 mg), 6-[(5-amino-1H-indol-1-yl)methyl]-N-(tert-butyl)pyridine-2-carboxamide (160 mg), isopropyl alcohol (8.0 mL), 1N aqueous sodium hydroxide solution (2.0 mL) and tetrahydrofuran (4.0 mL) and in the same manner as in Example G-10(v), the title compound (137 mg) was obtained as colorless crystals.

¹H-NMR (CDCl₃) δ: 1.50 (9H, s), 4.05-4.15 (2H, m), 4.30-4.40 (2H, m), 5.46 (2H, s), 6.16 (1H, d, J=3.0 Hz), 6.57 (1H, d, J=3.0 Hz), 6.85-6.90 (2H, m), 7.19 (1H, d, J=3.0 Hz), 7.20-7.30 (1H, m), 7.35 (1H, d, J=9.0 Hz), 7.69 (1H, t, J=7.5 Hz), 7.81 (1H, s), 7.92 (1H, s), 8.04 (1H, d, J=7.5 Hz), 8.20 (1H, s), 9,15 (1H, br s).

### Example G-19

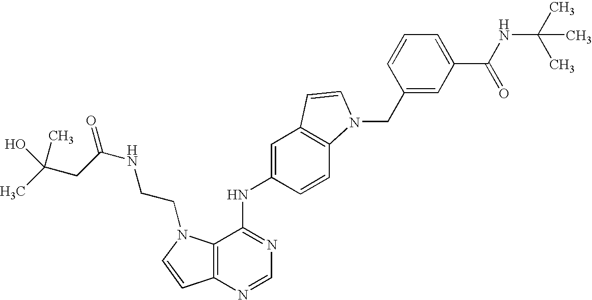

### Production of N-(tert-butyl)-3-({5-[(5-{2-[(3-hydroxy-3-methylbutanoyl)amino]ethyl}-5H-pyrrolo[3,2-d]pyrimidin-4-yl)amino]-1H-indol-1-yl}methyl)benzamide

### (i) Production of tert-butyl(2-{4-[(1-{3-[(tert-butylamino)carbonyl]benzyl}-1H-indol-5-yl)amino]-5H-pyrrolo[3,2-d]pyrimidin-5-yl}ethyl)carbamate

Using tert-butyl[2-(4-chloro-5H-pyrrolo[3,2-d]pyrimidin-5-yl)ethyl]carbamate (297 mg), 3-[(5-amino-1H-indol-1-yl)methyl]-N-(tert-butyl)benzamide (354 mg) and isopropyl alcohol (8.0 mL) and in the same manner as in Example G-8(iii), the title compound (478 mg) was obtained as colorless crystals.

¹H-NMR (CDCl₃) δ: 1.43 (9H, s), 1.46 (9H, s), 3.45-3.55 (2H, m), 4.35-4.50 (2H, m), 4.93 (1H, br s), 5.33 (2H, s), 5.88 (1H, s), 6.49 (1H, d, J=3.0 Hz), 6.55 (1H, d, J=2.4 Hz), 7.10-7.30 (4H, m), 7.30 (1H, t, J=7.8 Hz), 7.40-7.50 (1H, m), 7.54 (1H, d, J=8.1 Hz), 7.64 (1H, s), 7.86 (1H, s), 8.16 (1H, br s), 8.43 (1H, s).

### (ii) Production of 3-[(5-{[5-(2-aminoethyl)-5H-pyrrolo[3,2-d]pyrimidin-4-yl]amino}-1H-indol-1-yl)methyl]-N-(tert-butyl)benzamide dihydrochloride

Using tert-butyl(2-{4-[(1-{3-[(tert-butylamino)carbonyl]benzyl}-1H-indol-5-yl)amino]-5H-pyrrolo[3,2-d]pyrimidin-5-yl}ethyl)carbamate (449 mg) and 10% (W/W) hydrochloric acid/methanol (5.0 mL) and in the same manner as in Example G-8(iv), the title compound (400 mg) was obtained as a pale-orange powder.

¹H-NMR (DMSO-d₆) δ: 1.36 (9H, s), 3.25-3.35 (2H, m), 4.90-5.05 (2H, m), 5.51 (2H, s), 6.56 (1H, d, J=3.0 Hz), 6.69 (1H, d, J=3.3 Hz), 7.20 (1H, d, J=9.0 Hz), 7.29 (1H, d, J=7.2 Hz), 7.37 (1H, t, J=7.8 Hz), 7.51 (1H, d, J=9.0 Hz), 7.62 (1H, d, J=3.0 Hz), 7.65-7.70 (2H, m), 7.68 (1H, s), 7.99 (1H, d, J=3.0 Hz), 8.15.-8.30 (3H, m), 8.58 (1H, s), 9.89 (1H, s).

### (iii) Production of N-(tert-butyl)-3-({5-[(5-{2-[(3-hydroxy-3-methylbutanoyl)amino]ethyl}-5H-pyrrolo[3,2-d]pyrimidin-4-yl)amino]-1H-indol-1-yl}methyl)benzamide

Using 3-[(5-{[5-(2-aminoethyl)-5H-pyrrolo[3,2-d]pyrimidin-4-yl]amino}-1H-indol-1-yl)methyl]-N-(tert-butyl)benzamide dihydrochloride (150 mg), 3-hydroxy-3-methylbutanoic acid (48 mg), 1-hydroxybenzotriazole (59 mg), triethylamine (0.38 mL), 1-ethyl-3-(3-dimethylaminopropyl)carbodiimide hydrochloride (83 mg) and N,N-dimethylformamide (4.0 mL) and in the same manner as in Example G-8(v), the title compound (80 mg) was obtained as pale-yellow crystals.

¹H-NMR (CDCl₃) δ: 1.25 (6H, s), 1.46 (9H, s), 2.44 (2H, s), 3.55-3.65 (2H, m), 4.45-4.55 (2H, m), 5.30 (2H, s), 5.99 (1H, s), 6.51 (1H, d, J=3.0 Hz), 6.57 (1H, d, J=3.3 Hz), 7.10-7.35 (6H, m), 7.55 (1H, d, J=8.1 Hz), 7.64 (1H, s), 7.75-7.80 (1H, m), 8.05-8.10 (1H, m), 8.28 (1H, s), 9.55-9.65 (1H, br).

### Example G-20

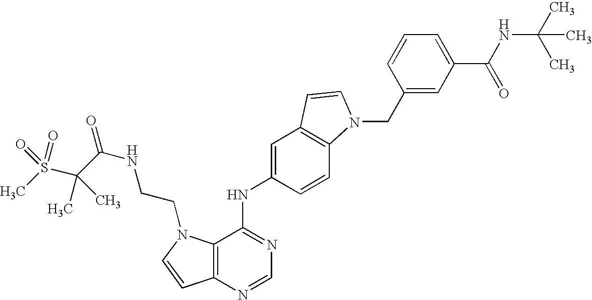

### Production of N-(tert-butyl)-3-[(5-{[5-(2-{[2-methyl-2-(methylsulfonyl)propanoyl]amino}ethyl)-5H-pyrrolo[3,2-d]pyrimidin-4-yl]amino}-1H-indol-1-yl)methyl]benzamide

Using 3-[(5-{[5-(2-aminoethyl)-5H-pyrrolo[3,2-d]pyrimidin-4-yl]amino}-1H-indol-1-yl)methyl]-N-(tert-butyl)benzamide dihydrochloride (150 mg), 2-methyl-2-(methylsulfonyl)propanoic acid (68 mg), 1-hydroxybenzotriazole (59 mg), triethylamine (0.38 mL), 1-ethyl-3-(3-dimethylaminopropyl)carbodiimide hydrochloride (83 mg) and N,N-dimethylformamide (4.0 mL) and in the same manner as in Example G-8(v), the title compound (91 mg) was obtained as colorless crystals.

¹H-NMR (CDCl₃) δ: 1.46 (6H, s), 1.60 (9H, s), 2.84 (3H, s), 3.60-3.75 (2H, m), 4.30-4.45 (2H, m), 5.34 (2H, s), 5.90 (1H, s), 6.51 (1H, d, J=2.7 Hz), 6.55-6.60 (1H, m), 7.00-7.10 (1H, m), 7.10-7.40 (6H, m), 7.53 (1H, d, J=7.5 Hz), 7.65 (1H, s), 7.81 (1H, s), 7.89 (1H, s), 8.44 (1H, s).

### Example H-1

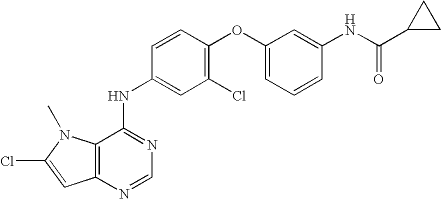

### Production of N-(3-{2-chloro-4-[(6-chloro-5-methyl-5H-pyrrolo[3,2-d]pyrimidin-4-yl)amino]phenoxy}phenyl)cyclopropanecarboxamide

### (i) Production of 4,6-dichloro-5-methyl-5H-pyrrolo[3,2-d]pyrimidine

To a solution of diisopropylamine (540 mg) in tetrahydrofuran (12 mL) was added n-butyllithium (2.7 mL) at 0° C. After stirring for 30 min, the mixture was cooled to −78° C., and 4-chloro-5-methyl-5H-pyrrolo[3,2-d]pyrimidine (500 mg) was added thereto. The reaction mixture was stirred for 1 hr, p-toluenesulfonyl chloride (690 mg) was added thereto, and the mixture was allowed to warm to −40° C. over 1 hr. Water was added to the reaction mixture and the mixture was extracted with ethyl acetate. The organic layer was washed successively with water and saturated brine, and dried over magnesium sulfate. After concentration under reduced pressure, the residue was separated and purified by basic silica gel column chromatography (eluent, hexane:ethyl acetate=80:20→30:70). After concentration under reduced pressure, the resulting crystals were collected by filtration, and washed with diisopropyl ether to give the title compound (191 mg) as crystals.

¹H-NMR (CDCl₃) δ: 4.13 (3H, s), 6.72 (1H, s), 8.68 (1H, s).

### (ii) Production of N-(3-{2-chloro-4-[(6-chloro-5-methyl-5H-pyrrolo[3,2-d]pyrimidin-4-yl)amino]phenoxy}phenyl)cyclopropanecarboxamide

A mixture of 4,6-dichloro-5-methyl-5H-pyrrolo[3,2-d]pyrimidine (98 mg), N-[3-(4-amino-2-chlorophenoxy)phenyl]cyclopropanecarboxamide (167 mg) and isopropyl alcohol (7.0 mL) was stirred at 80° C. for 3 hr. The reaction mixture was concentrated under reduced pressure, water and saturated aqueous sodium hydrogencarbonate were added thereto and the mixture was extracted with ethyl acetate. The organic layer was washed with saturated brine and dried over anhydrous magnesium sulfate. The solvent was evaporated under reduced pressure and the obtained residue was subjected to silica gel column chromatography (eluent, hexane:ethyl acetate=80:20→0:100). The objective fractions were concentrated under reduced pressure. The residue was crystallized from ethyl acetate-hexane to give the title compound (146 mg) as a white powder.

¹H-NMR (CDCl₃) δ: 0.78-0.84 (2H, m), 0.99-1.05 (2H, m), 1.47-1.54 (1H, m), 4.07 (3H, s), 6.60 (1H, s), 6.75-6.77 (2H, m), 7.01-7.04 (2H, m), 7.20-7.26 (3H, m), 7.62 (1H, s), 8.10 (1H, br s), 8.47 (1H, s).

### Example H-2

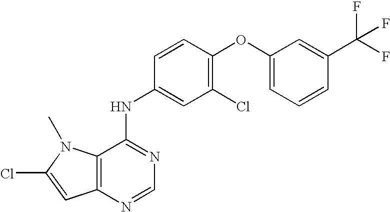

### Production of 6-chloro-N-{3-chloro-4-[3-(trifluoromethyl)phenoxy]phenyl}-5-methyl-5H-pyrrolo[3,2-d]pyrimidin-4-amine

Using 4,6-dichloro-5-methyl-5H-pyrrolo[3,2-d]pyrimidine (70 mg), 3-chloro-4-[3-(trifluoromethyl)phenoxy]aniline (109 mg) and isopropyl alcohol (7.0 mL) and in the same manner as in Example H-1(ii), the title compound (61 mg) was obtained as crystals.

¹H-NMR (DMSO-d₆) δ: 4.07 (3H, s), 6.73 (1H, s), 7.21-7.33 (3H, m), 7.46-7.67 (3H, m), 7.93 (1H, br s), 8.40 (1H, br s), 8.97 (1H, br s).

### Example H-3

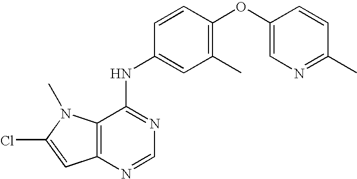

### Production of 6-chloro-5-methyl-N-{3-methyl-4-[(6-methylpyridin-3-yl)oxy]phenyl}-5H-pyrrolo[3,2-d]pyrimidin-4-amine

Using 4,6-dichloro-5-methyl-5H-pyrrolo[3,2-d]pyrimidine (150 mg), 3-methyl-4-[(6-methylpyridin-3-yl)oxy]aniline (160 mg) and isopropyl alcohol (8.0 mL) and in the same manner as in Example H-1(ii), the title compound (116 mg) was obtained as crystals.

¹H-NMR (DMSO-d₆) δ: 2.17 (3H, s), 2.43 (3H, s), 4.04 (3H, s), 6.65 (1H, s), 6.94 (1H, d, J=8.3 Hz), 7.16-7.25 (2H, m), 7.44-7.51 (2H, m), 7.16 (1H, d, J=2.7 Hz), 8.28 (1H, s), 8.57 (1H, s).

### Example H-4

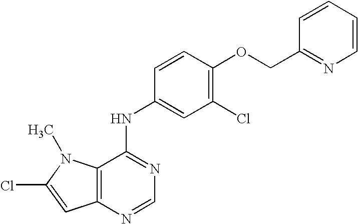

### Production of 6-chloro-N-[3-chloro-4-(pyridin-2-ylmethoxy)phenyl]-5-methyl-5H-pyrrolo[3,2-d]pyrimidin-4-amine

Using 4,6-dichloro-5-methyl-5H-pyrrolo[3,2-d]pyrimidine (150 mg), 3-chloro-4-(pyridin-2-ylmethoxy)aniline (210 mg) and isopropyl alcohol. (10 mL) and in the same manner as in Example H-1(ii), the title compound (170 mg) was obtained as crystals.

¹H-NMR (DMSO-d₆) δ: 4.03 (3H, s), 5.27 (2H, s), 6.64 (1H, s), 7.20-7.91 (6H, m), 8.25 (1H, s), 8.54 (1H, br s), 8.58-8.62 (1H, m).

### Example H-5

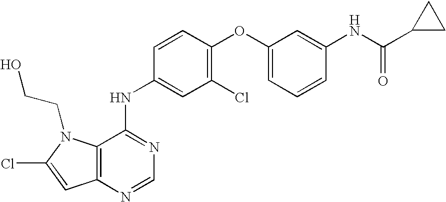

### Production of N-[3-(2-chloro-4-{[6-chloro-5-(2-hydroxyethyl)-5H-pyrrolo[3,2-d]pyrimidin-4-yl]amino}phenoxy)phenyl]cyclopropanecarboxamide

### (i) Production of 5-(2-{[tert-butyl(dimethyl)silyl]oxy}ethyl)-4-chloro-5H-pyrrolo[3,2-d]pyrimidine

4-Chloro-5H-pyrrolo[3,2-d]pyrimidine (2.00 g), (2-bromoethoxy)(tert-butyl)dimethylsilane (4.00 g) and cesium carbonate (6.40 g) were dissolved in N,N-dimethylformamide (10 mL) and the mixture was stirred at room temperature for 4 hr. Under ice-cooling, brine was added to the reaction mixture, and the mixture was extracted with ethyl acetate. The organic layer was dried over anhydrous magnesium sulfate and concentrated under reduced pressure. The residue was separated and purified by silica gel column chromatography (eluent, ethyl acetate:hexane=20:80→40:60) to give the title compound (3.02 g) as a brown solid.

¹H-NMR (DMSO-d₆) δ: −0.24 (6H, s), 0.69 (9H, s), 3.90-3.93 (2H, m), 4.61-4.64 (2H, m), 6.76 (1H, s), 8.00 (1H, s), 8.61 (1H, s).

### (ii) Production of N-[3-(2-chloro-4-{[6-chloro-5-(2-hydroxyethyl)-5H-pyrrolo[3,2-d]pyrimidin-4-yl]amino}phenoxy)phenyl]cyclopropanecarboxamide

Using diisopropylamine (2.20 g), 1.6 M n-butyllithium (14 mL), tetrahydrofuran (50 mL), 5-(2-{[tert-butyl(dimethyl)silyl]oxy}ethyl)-4-chloro-5H-pyrrolo[3,2-d]pyrimidine (500 mg), p-toluenesulfonyl chloride (6.10 g), N-[3-(4-amino-2-chlorophenoxy)phenyl]cyclopropanecarboxamide (190 mg) and isopropyl alcohol (10 mL) and in the same manner as in Example H-1(i) and (ii), a compound was obtained. The obtained compound was dissolved in methanol (10 mL), 4N hydrogen chloride/ethyl acetate solution (3.0 mL) was added thereto, and the mixture was stirred at 80° C. for 18 hr. Under ice-cooling, to the reaction mixture was added aqueous sodium bicarbonate, and the mixture was extracted with ethyl acetate. The organic layer was washed with brine and dried over anhydrous magnesium sulfate. The residue was separated and purified by silica gel column chromatography (eluent, ethyl acetate:methanol=100:0→95:5) to give the title compound (73 mg) as crystals.

¹H-NMR (DMSO-d₆) δ: 0.75-0.77 (4H, m), 1.69-1.75 (1H, m), 3.88-3.91 (2H, m), 4.53-4.55 (2H, m), 6.62-6.68 (1H, m), 6.72 (1H, s), 7.20-7.96 (6H, m), 8.37 (1H, s), 9.87-9.97 (1H, m), 10.24 (1H, s).

### Example H-6

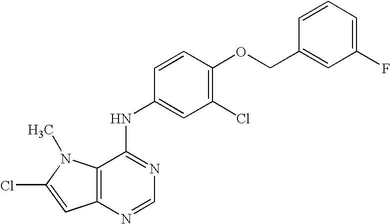

### Production of 6-chloro-N-{3-chloro-4-[(3-fluorobenzyl)oxy]phenyl}-5-methyl-5H-pyrrolo[3,2-d]pyrimidin-4-amine

Using 4,6-dichloro-5-methyl-5H-pyrrolo[3,2-d]pyrimidine (150 mg), 3-chloro-4-[(3-fluorobenzyl)oxy]aniline (167 mg) and isopropyl alcohol (10 mL) and in the same manner as in Example H-1(ii), the title compound (61 mg) was obtained as crystals.

¹H-NMR (DMSO-d₆) δ: 4.03 (3H, s), 5.24 (2H, s), 6.64 (1H, s), 7.14-7.50 (6H, m), 7.71-7.72 (1H, m), 8.26 (1H, s), 8.52 (1H, s).

### Example H-7

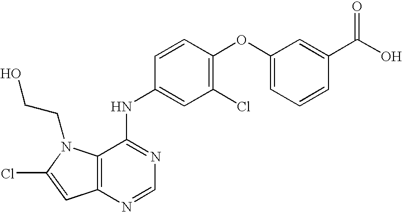

### Production of 3-(2-chloro-4-{[6-chloro-5-(2-hydroxyethyl)-5H-pyrrolo[3,2-d]pyrimidin-4-yl]amino}phenoxy)benzoic acid

Using diisopropylamine (2.00 g), 1.6M n-butyllithium (13 mL), tetrahydrofuran (50 mL), 5-(2-{[tert-butyl(dimethyl)silyl]oxy}ethyl)-4-chloro-5H-pyrrolo[3,2-d]pyrimidine (450 mg), p-toluenesulfonyl chloride (6.01 g), methyl 3-(4-amino-2-chlorophenoxy)benzoate (190 mg), isopropyl alcohol (10 mL), methanol (10 mL) and 4N hydrogen chloride/ethyl acetate solution (3.0 mL) and in the same manner as in Example H-1(i), (ii) and Example H-5(ii), a compound was obtained. To the obtained compound were added 1N aqueous sodium hydroxide solution (0.8 mL) and tetrahydrofuran (4.0 mL) and the mixture was stirred at room temperature for 2 days. The reaction mixture was neutralized with 1N hydrochloric acid and aqueous sodium bicarbonate and brine were added thereto. The mixture was extracted with ethyl acetate, and the organic layer was dried over anhydrous magnesium sulfate and concentrated under reduced pressure. The residue was separated and purified by silica gel column chromatography (eluent, ethyl acetate:methanol=100:0→95:5) to give the title compound (127 mg) as crystals.

¹H-NMR (DMSO-d₆) δ: 3.83-3.91 (2H, m), 4.54-4.57 (2H, m), 6.49 (1H, br s), 6.73 (1H, s), 7.24-7.68 (6H, m), 7.98 (1H, s), 9.97 (1H, s), 13.14 (1H, br s).

### Example H-8

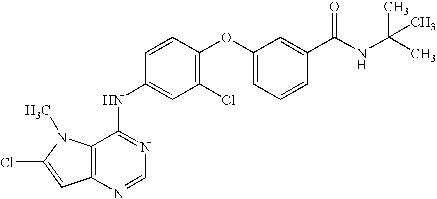

### Production of N-(tert-butyl)-3-{2-chloro-4-[(6-chloro-5-methyl-5H-pyrrolo[3,2-d]pyrimidin-4-yl)amino]phenoxy}benzamide

Using 4,6-dichloro-5-methyl-5H-pyrrolo[3,2-d]pyrimidine (110 mg), 3-(4-amino-2-chlorophenoxy)-N-(tert-butyl)benzamide (197 mg) and isopropyl alcohol (10 mL) and in the same manner as in Example H-1(ii), the title compound (86 mg) was obtained as crystals.

¹H-NMR (DMSO-d₆) δ: 1.36 (9H, s), 4.06 (3H, s), 6.69 (1H, s), 7.06-7.64 (6H, m), 7.81 (1H, s), 7.89 (1H, br s), 8.34 (1H, s), 8.81 (1H, s).

### Example H-9

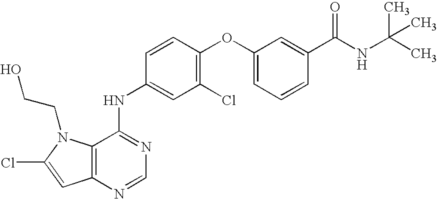

### Production of N-(tert-butyl)-3-(2-chloro-4-{[6-chloro-5-(2-hydroxyethyl)-5H-pyrrolo[3,2-d]pyrimidin-4-yl]amino}phenoxy)benzamide

Using diisopropylamine (2.80 g), 1.6M n-butyllithium (16 mL), tetrahydrofuran (50 mL), 5-(2-{[tert-butyl(dimethyl)silyl]oxy}ethyl)-4-chloro-5H-pyrrolo[3,2-d]pyrimidine (800 mg), p-toluenesulfonyl chloride (7.10 g), 3-(4-amino-2-chlorophenoxy)-N-(tert-butyl)benzamide (811 mg) and isopropyl alcohol (16 mL) and in the same manner as in Example H-1(i) and (ii), a compound was obtained. The obtained compound was dissolved in methanol (10 mL), 4N hydrogen chloride/ethyl acetate solution (10 mL) was added thereto, and the mixture was stirred at 80° C. for 18 hr. Under ice-cooling, to the reaction mixture was added aqueous sodium bicarbonate, and the mixture was extracted with ethyl acetate. The organic layer was washed with brine and dried over anhydrous magnesium sulfate. The residue was separated and purified by silica gel column chromatography (eluent, ethyl acetate:methanol=100:0→395:5) to give the title compound (576 mg) as crystals.

¹H-NMR (DMSO-d₆) δ: 1.36 (9H, s), 3.88-3.91 (2H, m), 4.53-4.57 (2H, m), 6.72 (1H, s), 7.06-7.61 (6H, m), 7.81 (1H, s), 7.96-7.97 (1H, m), 8.37 (1H, s), 9.52-10.53 (1H, m).

### Example H-10

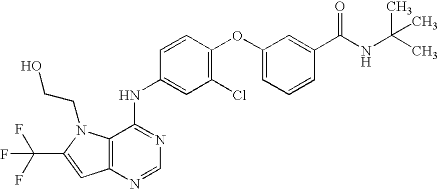

### Production of N-(tert-butyl)-3-(2-chloro-4-{[5-(2-hydroxyethyl)-6-(trifluoromethyl)-5H-pyrrolo[3,2-d]pyrimidin-4-yl]amino}phenoxy)benzamide

### (i) 5-(2-{[tert-butyl(dimethyl)silyl]oxy}ethyl)-4-chloro-6-(trifluoromethyl)-5H-pyrrolo[3,2-d]pyrimidine

### Production of

To a solution of diisopropylamine (300 mg) in tetrahydrofuran (20 mL) was added 1.6 M n-butyllithium (2.0 mL) at 0° C. After stirring for 30 min, the mixture was cooled to −78° C., and 5-(2-{[tert-butyl(dimethyl)silyl]oxy}ethyl)-4-chloro-5H-pyrrolo[3,2-d]pyrimidine (520 mg) was added thereto. The reaction mixture was stirred for 1 hr, S-(trifluoromethyl)dibenzothiophenium trifluoromethanesulfonate (2.00 g) was added thereto, and the mixture was allowed to warm to −40° C. over 1 hr. Water was added to the reaction mixture and the mixture was extracted with ethyl acetate. The organic layer was washed successively with water and saturated brine, and dried over magnesium sulfate. After concentration under reduced pressure, the residue was separated and purified by basic silica gel column chromatography (eluent, hexane:ethyl acetate=80:20→30:70). After concentration under reduced pressure, the resulting crystals were collected by filtration and washed with diisopropyl ether to give the title compound (29 Mg) as crystals.

¹H-NMR (CDCl₃) δ: 0.02 (6H, s), 0.74 (9H, s), 3.92-3.96 (2H, m), 4.74-4.78 (2H, m), 7.17. (1H, s), 8.79 (1H, s).

### (ii) Production of N-(tert-butyl)-3-(2-chloro-4-{[5-(2-hydroxyethyl)-6-(trifluoromethyl)-5H-pyrrolo[3,2-d]pyrimidin-4-yl]amino}phenoxy)benzamide

Using 5-(2-{[tert-butyl(dimethyl)silyl]oxy}ethyl)-4-chloro-6-(trifluoromethyl)-5H-pyrrolo[3,2-d]pyrimidine (25 mg), 3-(4-amino-2-chlorophenoxy)-N-(tert-butyl)benzamide (26 mg) and isopropyl alcohol (1.5 mL) and in the same manner as in Example H-2(ii), a compound was obtained. The obtained compound was dissolved in methanol (2.0 mL), 4N hydrogen chloride/ethyl acetate solution (1.0 mL) was added thereto, and the mixture was stirred at 80° C. for 18 hr. Aqueous sodium bicarbonate was added to the reaction mixture under ice-cooling, and the mixture was extracted with ethyl acetate. The organic layer was washed with brine and dried over anhydrous magnesium sulfate. The residue was separated and purified by silica gel column chromatography (eluent, ethyl acetate:methanol=100:0→95:5) to give the title compound (9.2 mg) as crystals.

¹H-NMR (CDCl₃) δ: 1.36 (9H, s), 3.92-3.96 (2H, m), 4.61-4.64 (2H, m), 7.06-7.45 (5H, m), 7.55-7.61 (2H, m), 7.82 (1H, s), 7.97 (1H, s), 8.44 (1H, s), 9.50-10.55 (1H, m).

### Formulation Example 1

### Amount Per Tablet

| | |
|---|---|
| (1) Compound obtained in Example A-1 | 10.0 mg |
| (2) Lactose | 60.0 mg |
| (3) Corn starch | 35.0 mg |
| (4) Gelatin | 3.0 mg |
| (5) Magnesium stearate | 2.0 mg |

A mixture of 10.0 mg of the compound obtained in Example A-1, 60.0 mg of lactose and 35.0 mg of corn starch is granulated through a 1 mm-mesh sieve using 0.03 ml of a 10% by weight aqueous solution of gelatin (3.0 mg of gelatin), after which the granules are dried at 40° C. and filtered again. The obtained granules are mixed with 2.0 mg of magnesium stearate and compressed. The obtained core tablets are coated with a sugar coat comprising a suspension of sucrose, titanium dioxide, talc and gum arabic and polished with beeswax to yield sugar-coated tablets.

### Formulation Example 2

### Dose Per Tablet

| | |
|---|---|
| (1) Compound obtained in Example A-1 | 10.0 mg |
| (2) Lactose | 70.0 mg |
| (3) Corn starch | 50.0 mg |
| (4) Soluble starch | 7.0 mg |
| (5) Magnesium stearate | 3.0 mg |

10.0 mg of the compound obtained in Example A-1 and 3.0 mg of magnesium stearate are granulated using 0.07 ml of an aqueous solution of soluble starch (7.0 mg of soluble starch), after which these granules are dried and mixed with 70.0 mg of lactose and 50.0 mg of corn starch. This mixture is compressed to yield tablets.

### Experimental Example 1A

### Cloning of Human HER2 Gene and Preparation of Recombinant Baculovirus

Human HER2 gene was cloned by RT-PCR using total RNA prepared from MCF7 cells as a template. The primer used for RT-PCR was prepared from nucleotide sequence (Genbank Accession No. M11730) information of HER2 gene by adding a nucleotide sequence encoding DYKDDDD peptide and a restriction enzyme recognition sequence to a nucleotide sequence (2176-3918 of Genbank Accession No. M11730) encoding the HER2 intracellular domain region, so that the protein contains an N-terminal DYKDDDD peptide tag. The primer nucleotide sequence is shown below.

(SEQ ID NO: 1)

HER2-U:
5′-AATTAAGTCGACATGGACTACAAAGACGATGACGACAAGCGACGGCA
GCAGAAGATCCGGAAGTAC-3′
and

(SEQ ID NO: 2)

HER2-L:
5′-AATTAAGCATGCTCACACTCGCACGTCCAGACCCAGGTACTC-3′

The RT reaction was conducted using SuperScript First-Strand Synthesis System for RT-PCR (Invitrogen) and the PCR reaction was conducted using a KOD-plus kit (TOYOBO). The obtained PCR product was electrophoresed on agarose gel (1%), the DNA fragment amplified by PCR was recovered from the gel, and then digested with restriction enzymes Sal I and Sph I. The DNA treated with the restriction enzymes was electrophoresed on agarose gel (1%), and the obtained DNA fragment was recovered and ligated to plasmid pFASTBAC1 (Invitrogen) digested with restriction enzymes Sal I and Sph I to give expression plasmid pFB-HER2. The nucleotide sequence of the insertion fragment was confirmed and found to be identical with the nucleotide sequence (2176-3918 of Genbank Accession M11730) of HER2 intracellular domain. Furthermore, using BAC-TO-BAC Baculovirus Expression System (Invitrogen), recombinant baculovirus BAC-HER2 was prepared.

### Experimental Example 1B

### Preparation of HER2 Intracellular Domain Protein

SF-21 cells were sown at 1×10⁶ cells/mL to Sf-900II SFM medium (1 L, Invitrogen) containing 10% fetal bovine serum (trace), 50 mg/L gentamicin (Invitrogen) and 0.1% Pluronic F-68 (Invitrogen), and shaking culture was performed using a 2 L volume Erlenmeyer flask at 27° C., 100 rpm. After culturing for 24 hr, recombinant baculovirus BAC-HER2 (13.4 mL) was added, and the mixture was further cultured for 3 days. The culture medium was centrifuged at 2,000 rpm for 5 min. to give virus-infected cells. The infected cells were washed with a phosphate buffered saline (Invitrogen), centrifuged under the same conditions, and the cells were preserved at −80° C. The cryopreserved cells were thawed in ice, suspended in buffer A (50 mM Tris buffer (30 mL, pH 7.4) containing 20% glycerol, 0.15 M NaCl) supplemented with Complete Protease Inhibitor (Boehringer), and ruptured 3 times with a Polytron homogenizer (Kinematica) at 20,000 rpm for 30 sec. The rupture medium was clarified by centrifugation at 40,000 rpm for 30 min. and filtered with a 0.45 μm filter. The filtrate was passed through a column packed with Anti-FLAG M2 Affinity Gel (4 mL, Sigma) at a flow rate of about 0.5 mL/min. The column was washed with buffer A, and eluted with buffer A containing 100 μg/mL of FLAG peptide. The eluate was concentrated with Vivaspin 20 (Vivascience) having a molecular weight cut off of 30K. The concentrate was purified by gel filtration using Hi Load Superdex 200pg 16/60 (Amersham Bioscience) equilibrated with buffer A. The fractions containing HER2 intracellular domain were collected, glycerol was added to the final concentration of 50% and cryopreserved at −80° C.

### Experimental Example 1C

### Determination of HER2 Kinase Inhibitory Activity

A test compound dissolved in dimethyl sulfoxide (DMSO) was diluted with a buffer for kinase reaction (50 mM Tris-HCl (pH7.5), 5 mM MgCl₂, 5 mM MnCl₂, 2 mM dithiothreitol, 0.01% Tween-20). To this compound solution (10 μL) was added a buffer for kinase reaction (20 μL) containing 5 μg/mL of HER2 intracellular domain obtained in Experimental Example 1B and 12.5 μg/mL of polypeptide substrate poly-Glu:Tyr (4:1) (Sigma). To the obtained mixture was added 20 μL of ATP solution (1.25 μM ATP, 0.05 μCi [γ-³²P]ATP), the mixture was allowed to react at 25° C. for 10 min. and the reaction was quenched with 50 μL of 20% TCA solution. The reaction solution was allowed to stand at 4° C. for 20 min., and the acid insoluble fraction was transferred to GF/C filter (PerkinElmer) using cell harvester (PerkinElmer) and washed with 250 mM phosphoric acid solution. After washing, the plate was dried at 45° C. for 60 min., and 35 μL of MicroScinti 0 (PerkinElmer) was added. The radioactivity was measured using TopCount (PerkinElmer). HER2 kinase inhibitory rate (%) of the test compound was calculated by the following formula:

Inhibitory rate (%)=(1−(count of test compound−blank)+(control−blank))×100

The count of the solution reacted without addition of the compound was used as a “control”, and the count of the solution without the compound and HER2 intracellular domain was used as a “blank”. The results of the inhibitory rate of the compounds are shown in Table 1.

From the foregoing, it was shown that the compounds of the present invention strongly inhibited the activity of HER2 kinase.

TABLE 1

| Example No. | Inhibitory rate (%) |
|---|---|
| (compound No.) | at 1.0 μM |
| A-1 | 98 |
| A-15 | 99 |
| B-1 | 99 |
| C-26 | 98 |
| C-45 | 97 |
| C-89 | 100 |
| E-22 | 99 |
| G-5 | 93 |
| G-14 | 100 |

### Experimental Example 2

### Inhibitory Action on Breast Cancer Cell BT-474 Proliferation in vitro

A suspension of human breast cancer cell. BT-474 (100 μl (6,000 cells)) were seeded to attach in a 96-well microplate and cultured in an incubator (37° C., 5% carbon dioxide). On the following day, 100 μl of a solution of each test compound previously diluted serially in 2-fold, was added to give indicated dose, and the cells were cultured for 5 days. After the culture medium containing the test compound was removed, the cells were washed and fixed with 50% trichloroacetic acid, after which a 0.4% (w/v) SRB solution (dissolved in 1% acetic acid) was added to stain the cell protein (Skehan et al., Journal of the National Cancer Institute, Vol. 82, pp. 1107-1112, 1990). After washing with a 1% acetic acid solution, 100 μl of Tris solution (10 mM) was added to extract the pigment and the absorbance was measured at a wavelength of 550 nm to quantify the amount of cells as protein content. Taking as 100% the protein content for the control group, which received no test compound solution, the ratio of the residual protein content for, each treatment group was determined, and the compound concentration required to achieve 50% suppression of the residual cell content relative to the control (IC₅₀ value) was calculated. The results are shown in Table 2.

TABLE 2

| Example No. | |
|---|---|
| (compound No.) | IC₅₀ (nM) |
| A-1 | <100 |
| C-45 | <100 |
| C-97 | <100 |
| C-116 | <100 |
| G-5 | <100 |

### INDUSTRIAL APPLICABILITY

According to the present invention, pyrrolo[3,2-d]pyrimidine and pyrazolo[4,3-d]pyrimidine compounds, a production method thereof and use thereof are provided. These fused pyrimidine compounds have a superior tyrosine kinase inhibitory action, are highly safe, and are sufficiently satisfactory as pharmaceutical products.

This application is based on patent application Nos. 2005-349858 and 2006-060648 filed in Japan, the contents of which are incorporated in full herein by this reference.

1. A compound represented by the formula: 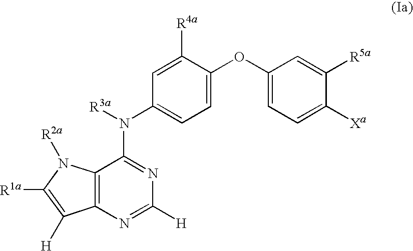
wherein
R^{1a} is a hydrogen atom,
R^{2a} is a C₁₋₆ alkyl group substituted by a group represented by —NR^{6a}—CO—(CH₂)ₙ—SO₂— optionally halogenated C₁₋₄ alkyl
wherein n is an integer of 1 to 4, R^{6a} is a hydrogen atom or a C₁₋₄ alkyl group, and —(CH₂)ₙ— is optionally substituted by C₁₋₄ alkyl,
R^{3a} is a hydrogen atom or a C₁₋₆, alkyl group,
R^{4a} is a halogen atom or a C₁₋₆ alkyl group,
R^{5a} is a halogen atom or a C₁₋₆ alkyl group, and
X^{a} is a hydrogen atom or a halogen atom,
or a salt thereof,
provided that N-[2-(4-{[3-chloro-4-(3-chlorophenoxy)phenyl]amino}-5H-pyrrolo[3,2-d]pyrimidin-5-yl)ethyl]-2-(methylsulfonyl)acetamide is excluded.

2. The compound of claim 1, wherein X^{a} is a hydrogen atom.

3. The compound of claim 2, wherein
R^{1a} is a hydrogen atom,
R^{2a} is a C₁₋₆ alkyl group substituted by a group represented by —NR^{6aa}—CO—CR^{7a}R^{8a}—SO₂—C₁₋₄ alkyl
wherein R^{6aa} is a hydrogen atom or a methyl group, R^{7a} and R^{8a} are the same or different and each is a hydrogen atom or a methyl group,
R^{3a} is a hydrogen atom,
R^{4a} is a chlorine atom or a methyl group, and
R^{5a} fluorine atom, a chlorine atom or a methyl group.

4. The compound of claim 3, wherein R^{7a} and R^{8a} are methyl groups.

5. A compound selected from the following:
N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide,
N-[2-(4-{[3-chloro-4-(3-chlorophenoxy)phenyl]amino}-5H-pyrrolo[3,2-d]pyrimidin-5-yl)ethyl]-2-(ethylsulfonyl)acetamide,
N-[2-(4-{[3-chloro-4-(3-chlorophenoxy)phenyl]amino}-5H-pyrrolo[3,2-d]pyrimidin-5-yl)ethyl]-N,2-dimethyl-2-(methylsulfonyl)propanamide,
N-[2-(4-{[3-chloro-4-(3-methylphenoxy)phenyl]amino}-5H-pyrrolo[3,2-d]pyrimidin-5-yl)ethyl]-2-(methylsulfonyl)acetamide,
N-[2-(4-{[3-chloro-4-(3-fluorophenoxy)phenyl]amino}-5H-pyrrolo[3,2-d]pyrimidin-5-yl)ethyl]-2-(methylsulfonyl)acetamide, and
N-[2-(4-{[4-(3-chlorophenoxy)-3-methylphenyl]amino}-5H-pyrrolo[3,2-d]pyrimidin-5-yl)ethyl]-2-methyl-2-(methylsulfonyl)propanamide,
or a salt thereof, or a hydrate thereof.

6. A compound represented by the formula: 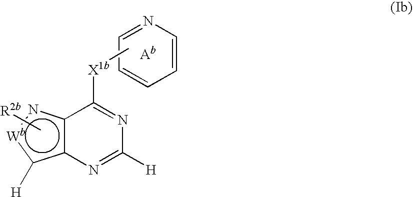
wherein
W^{b} is C(R^{1b}) or N,
ring A^{b} is an optionally substituted pyridine ring,
X^{1b} is —NR^{3b}—Y^{1b}—, —O—, —S—, —SO—, —SO₂— or —CHR^{3b}—
wherein R^{3b} is a hydrogen atom or an optionally substituted aliphatic hydrocarbon group, or R^{3b} is optionally bonded to the carbon atom on the pyridine ring for ring A^{b} to form an optionally substituted ring structure, and Y^{1b} is a bond, or a C₁₋₄ alkylene or —O—(C₁₋₄ alkylene)-, each of which is optionally substituted, and
R^{1b} is a hydrogen atom, a halogen atom, or an optionally substituted group bonded via a carbon atom, a nitrogen atom or an oxygen atom,
R^{2b} is a hydrogen atom, or an optionally substituted group bonded via a carbon atom or a sulfur atom, or
R^{1b} and R^{2b}, or R^{2b} and R^{3b} are optionally bonded to form an optionally substituted ring structure,
or a salt thereof.

7. The compound of claim 6, which is a compound represented by the formula: 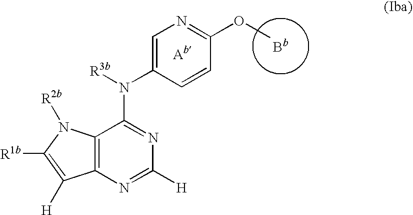
wherein ring A^{b′} is an optionally further substituted pyridine ring, ring B^{b} is an optionally substituted C₆₋₁₄ aryl group, and the other symbols are as defined in claim 6.

8. The compound of claim 7, wherein
R^{1b} is a hydrogen atom, a halogen atom, a cyano group or an optionally halogenated C₁₋₆ alkyl group,
R^{2b} is a C₁₋₆ alkyl group substituted by substituent(s) selected from the group consisting of
(i) —NR^{6ba}—CO—(CH₂)ₙ₁—SO₂—C₁₋₄ alkyl
wherein R^{6ba} is a hydrogen atom or a methyl group, n1 is an integer of 1 to 4, and —(CH₂)ₙ₁— is optionally substituted by C₁₋₄ alkyl,
(ii) —NR^{6bb}—CO—(CH₂)ₙ₂—OH
wherein R^{6bb} is a hydrogen atom or a methyl group, n2 is an integer of 1 to 4, and —(CH₂)ₙ₂— is optionally substituted by C₁₋₄ alkyl,
(iii) —O—(CH₂)ₙ₃—OH
wherein n3 is an integer of 1 to 4, and —(CH₂)ₙ₃— is optionally substituted by C₁₋₄ alkyl, and
(iv) hydroxy,
R^{3b} is a hydrogen atom,
ring A^{b′} is a pyridine ring optionally substituted by substituent(s) selected from the group consisting of halogen and methyl, and
ring B^{b} is a phenyl group optionally substituted by substituent(s) selected from the group consisting of optionally halogenated C₁₋₆ alkyl, optionally halogenated C₁₋₆ alkoxy, C₁₋₆ alkyl-carbamoyl and halogen.

9. The compound of claim 7, wherein
ring A^{b′} is a pyridine ring optionally substituted by halogen, and
ring B^{b} is a phenyl group optionally substituted at the 3-position by substituent(s) selected from the group consisting of optionally halogenated C₁₋₆ alkyl, optionally halogenated C₁₋₆ alkoxy, C₁₋₆ alkyl-carbamoyl and halogen.

10. A compound selected from the following:
2-{2-[4-({5-chloro-6-[3-(trifluoromethyl)phenoxy]pyridin-3-yl}amino)-5H-pyrrolo[3,2-d]pyrimidin-5-yl]ethoxy}ethanol,
N-{2-[4-({5-chloro-6-[3-(trifluoromethyl)phenoxy]pyridin-3-yl}amino)-5H-pyrrolo[3,2-d]pyrimidin-5-yl]ethyl}-2-(methylsulfonyl)acetamide,
N-{2-[4-({5-chloro-6-[3-(trifluoromethyl)phenoxy]pyridin-3-yl}amino)-5H-pyrrolo[3,2-d]pyrimidin-5-yl]ethyl}-3-hydroxy-3-methylbutanamide,
N-{2-[4-({5-chloro-6-[3-(trifluoromethoxy)phenoxy]pyridin-3-yl}amino)-5H-pyrrolo[3,2-d]pyrimidin-5-yl]ethyl}-2-(methylsulfonyl)acetamide, and
N-(tert-butyl)-3-[(3-chloro-5-{[5-(2-hydroxyethyl)-5H-pyrrolo[3,2-d]pyrimidin-4-yl]amino}pyridin-2-yl)oxy]benzamide,
or a salt thereof.

11. A compound represented by the formula: 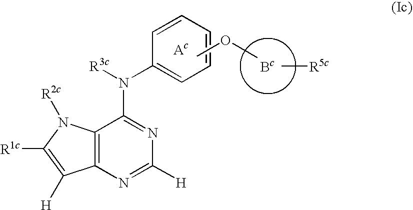
wherein
R^{1c} is a hydrogen atom, or an optionally substituted group bonded via a carbon atom, a nitrogen atom or an oxygen atom,
R^{2c} is an optionally substituted group bonded via a carbon atom or a sulfur atom, or
R^{1c} and R^{2c}, or R^{2c} and R^{3c} are optionally bonded to form an optionally substituted ring structure,
R^{3c} is a hydrogen atom or an optionally substituted aliphatic hydrocarbon group, or R^{3c} is optionally bonded to the carbon atom on the adjacent benzene ring to form an optionally substituted ring structure,
ring A^{c} is an optionally substituted benzene ring,
R^{5c} is
(i) an optionally substituted amino group,
(ii) an optionally substituted carbamoyl group,
(iii) an optionally substituted ureido group,
(iv) an optionally substituted sulfamoyl group,
(v) an optionally substituted heterocyclic group,
(vi) an optionally substituted C₂₋₆ alkoxy group
(vii) an optionally substituted aminomethyl group,
(viii) an optionally substituted carbamoylmethyl group,
(ix) an optionally substituted alkylsulfonyl group, or
(x) a cyano group, and
ring B^{c} is a C₆₋₁₄ aryl group or a C₅₋₈ cycloalkyl group, each of which is optionally further substituted besides R^{5c},
or a salt thereof,
provided that
N-(tert-butyl)-4-[2-chloro-4-({5-[2-(2-hydroxyethoxy)ethyl]-5H-pyrrolo[3,2-d]pyrimidin-4-yl}amino)phenoxy]benzamide hydrochloride,
4-[2-chloro-4-({5-[2-(2-hydroxyethoxy)ethyl]-5H-pyrrolo[3,2-d]pyrimidin-4-yl}amino)phenoxy]-N-(2,2-dimethylpropyl)benzamide,
3-(2-chloro-4-{[5-(2-hydroxyethyl)-5H-pyrrolo[3,2-d]pyrimidin-4-yl]amino}phenoxy)benzonitrile,
3-[2-chloro-4-({5-[2-(2-hydroxyethoxy)ethyl]-5H-pyrrolo[3,2-d]pyrimidin-4-yl}amino)phenoxy]benzonitrile,
3-[2-chloro-4-(6,7-dihydro-9H-pyrimido[4′,5′:4,5]pyrrolo[2,1-c][1,4]oxazin-4-ylamino)phenoxy]benzonitrile hydrochloride, and
(2E)-N-[(2E)-3-(4-{[3-chloro-4-(3-cyanophenoxy)phenyl]amino}-5-methyl-5H-pyrrolo[3,2-d ]pyrimidin-6-yl)prop-2-en-1-yl]-4-(dimethylamino)but-2-enamide are excluded.

12. The compound of claim 11, wherein R^{1c} is a hydrogen atom.

13. A compound selected from the following:
2-{2-[4-({3-chloro-4-[3-(1,3-thiazol-5-yl)phenoxy]phenyl}amino)-5H-pyrrolo[3,2-d]pyrimidin-5-yl]ethoxy}ethanol,
N-(tert-butyl)-3-[2-chloro-4-({5-[2-(2-hydroxyethoxy)ethyl]-5H-pyrrolo[3,2-d]pyrimidin-4-yl}amino)phenoxy]benzamide,
3-[2-chloro-4-({5-[2-(2-hydroxyethoxy)ethyl]-5H-pyrrolo[3,2-d]pyrimidin-4-yl}amino)phenoxy]-N-(2-hydroxy-1,1-dimethylethyl)benzamide,
N-(tert-butyl)-3-(2-chloro-4-{[5-(2-hydroxyethyl)-5H-pyrrolo[3,2-d]pyrimidin-4-yl]amino}phenoxy)benzamide,
N-(3-{2-chloro-4-[(6-cyano-5-methyl-5H-pyrrolo[3,2-d]pyrimidin-4-yl)amino]phenoxy}phenyl)cyclopropanecarboxamide,
N-(tert-butyl)-5-(2-chloro-4-{[5-(2-hydroxyethyl)-5H-pyrrolo[3,2-d]pyrimidin-4-yl]amino}phenoxy)-2-fluorobenzamide,
N-{2-[4-({3-chloro-4-[3-(dimethylamino)phenoxy]phenyl}amino)-5H-pyrrolo[3,2-d]pyrimidin-5-yl]ethyl}-3-hydroxy-3-methylbutanamide,
N-{2-[4-({3-chloro-4-[3-(dimethylamino)phenoxy]phenyl}amino)-5H-pyrrolo[3,2-d]pyrimidin-5-yl]ethyl}-2-(methylsulfonyl)acetamide,
N-(tert-butyl)-2-[3-(2-chloro-4-{[5-(2-hydroxyethyl)-5H-pyrrolo[3,2-d]pyrimidin-4-yl]amino}phenoxy)phenyl]acetamide,
N-{2-[4-({3-chloro-4-[3-(cyclopropylmethoxy)phenoxy]phenyl}amino)-5H-pyrrolo[3,2-d]pyrimidin-5-yl]ethyl}-2-(methylsulfonyl)acetamide,
N-{2-[4-({3-chloro-4-[3-(2,2-dimethylpropoxy)phenoxy]phenyl}amino)-5H-pyrrolo[3,2-d]pyrimidin-5-yl]ethyl}-2-(methylsulfonyl)acetamide,
2-(methylsulfonyl)-N-{2-[4-({3-methyl-4-[3-(2,2,2-trifluoroethoxy)phenoxy]phenyl}amino)-5H-pyrrolo[3,2-d]pyrimidin-5-yl]ethyl}acetamide,
2-[4-({3-chloro-4-[3-(isopropylsulfonyl)phenoxy]phenyl}amino)-5H-pyrrolo[3,2-d]pyrimidin-5-yl]ethanol, and
N-[2-(4-{[3-chloro-4-(3-cyanophenoxy)phenyl]amino}-5H-pyrrolo[3,2-d]pyrimidin-5-yl)ethyl]-2-(methylsulfonyl)acetamide,
or a salt thereof.

14. A compound represented by the formula: 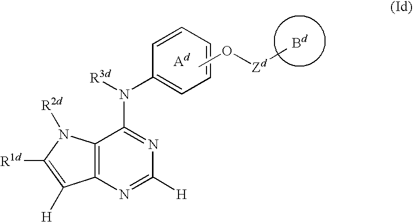
wherein
R^{1d} is a hydrogen atom, or an optionally substituted group bonded via a carbon atom, a nitrogen atom or an oxygen atom,
R^{2d} is an optionally substituted group bonded via a carbon atom or a sulfur atom, or,
R^{1d} and R^{2d}, or R^{2d} and R^{3d} are optionally bonded to form an optionally substituted ring structure,
R^{3d} is a hydrogen atom or an optionally substituted aliphatic hydrocarbon group, or R^{3d} is optionally bonded to the carbon atom on the adjacent benzene ring to form an optionally substituted ring structure,
ring A^{d} is an optionally substituted benzene ring,
Z^{d} is an optionally substituted C₁₋₃ alkylene,
ring B^{d} is an optionally substituted heterocyclic group,
or a salt thereof,
provided that
ethyl 5-[(4-{[3-chloro-4-(pyridin-2-ylmethoxy)phenyl]amino}-5H-pyrrolo[3,2-d]pyrimidin-5-yl)methyl]-2-furoate,
5-[(4-{[3-chloro-4-(pyridin-2-ylmethoxy)phenyl]amino}-5H-pyrrolo[3,2-d]pyrimidin-5-yl)methyl]-2-furancarboxylic acid,
2-[2-(4-{[3-chloro-4-(pyridin-2-ylmethoxy)phenyl]amino}-5H-pyrrolo[3,2-d]pyrimidin-5-yl)ethoxy]ethanol, and
N-[2-(4-{[3-chloro-4-(pyridin-2-ylmethoxy)phenyl]amino}-5H-pyrrolo[3,2-d]pyrimidin-5-yl)ethyl]-2-(methylsulfonyl)acetamide
are excluded.

15. The compound of claim 14, which is a compound represented by the formula: 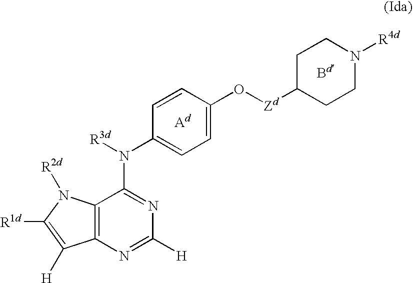
wherein R^{4d} is an acyl group or an optionally substituted ureido group, ring B^{d′} is a piperidyl group optionally further substituted besides R^{4d}, and the other symbols are as defined in claim 14.

16. A compound selected from the following:
tert-butyl 4-{[2-chloro-4-({5-[2-(2-hydroxyethoxy)ethyl]-5H-pyrrolo[3,2-d]pyrimidin-4-yl}amino)phenoxy]methyl}piperidine-1-carboxylate, and
tert-butyl 4-[(2-chloro-4-{[5-(2-hydroxyethyl)-5H-pyrrolo[3,2-d]pyrimidin-4-yl]amino}phenoxy)methyl]piperidine-1-carboxylate,
or a salt thereof.

17. A compound represented by the formula: 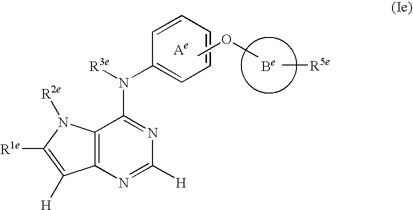
wherein
R^{1e} is a hydrogen atom, or an optionally substituted group bonded via a carbon atom, a nitrogen atom or an oxygen atom,
R^{2e} is an optionally substituted group bonded via a carbon atom or a sulfur atom, or,
R^{1e} and R^{2e}, or R^{2e} and R^{3e} are optionally bonded to form an optionally substituted ring structure,
R^{3c} is a hydrogen atom or an optionally substituted aliphatic hydrocarbon group, or R^{3e} is optionally bonded to the carbon atom on the adjacent benzene ring to form an optionally substituted ring structure,
ring A^{e} is an optionally substituted benzene ring,
R^{5e} is
(i) a linear alkyl group substituted by optionally substituted heterocyclic group,
(ii) a linear alkyl group substituted by optionally substituted imino,
(iii) a linear alkyl group substituted by optionally substituted aryl, which is optionally further halogenated or hydroxylated,
(iv) an optionally substituted branched alkyl group,
(v) an optionally substituted alkenyl group,
(vi) a hydroxy group substituted by optionally substituted aryl,
(vii) a hydroxy group substituted by C₁₋₆ alkyl,
(viii) a hydroxy group substituted by halogenated C₂₋₆ alkyl,
(ix) a halogenated C₂₋₆ alkyl group,
(x) an optionally substituted cycloalkyl group, or
(xi) a C₁₋₆ alkyl-carbonyl group optionally substituted by optionally substituted aryl, and ring B^{e} is a C₆₋₁₄ aryl group optionally further substituted besides R^{5e},
or a salt thereof,
provided that
2-(2-{4-[(3-chloro-4-{4-[3-(1H-imidazol-1-yl)propyl]phenoxy}phenyl)amino]-5H-pyrrolo[3,2-d]pyrimidin-5-yl}ethoxy)ethanol dihydrochloride,
2-(2-{4-[(3-chloro-4-{4-[4-(1H-1,2,3-triazol-1-yl)butyl]phenoxy}phenyl)amino]-5H-pyrrolo[3,2-d]pyrimidin-5-yl}ethoxy)ethanol, and
1-{3-[2-chloro-4-({5-[2-(2-hydroxyethoxy)ethyl]-5H-pyrrolo[3,2-d]pyrimidin-4-yl}amino)phenoxy]phenyl}ethanone
are excluded.

18. A compound selected from the following:
2-[4-({3-chloro-4-[3-(1,1-difluoroethyl)phenoxy]phenyl}amino)-5H-pyrrolo[3,2-d]pyrimidin-5-yl]ethanol,
(1Z)-1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one O-ethyloxime,
1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-ol,
1-[3-(2-chloro-4-{[5-(2-hydroxyethyl)-5H-pyrrolo[3,2-d]pyrimidin-4-yl]amino}phenoxy)phenyl]-3,3-dimethylbutan-1-one,
N-(2-{4-[(3-methyl-4-{3-[(1E)-3-methylbut-1-en-1-yl]phenoxy}phenyl)amino]-5H-pyrrolo[3,2-d]pyrimidin-5-yl}ethyl)-2-(methylsulfonyl)acetamide, and
N-{2-[4-({3-chloro-4-[3-(1-cyanocyclopropyl)phenoxy]phenyl}amino)-5H-pyrrolo[3,2-d]pyrimidin-5-yl]ethyl}-2-(methylsulfonyl)acetamide,
or a salt thereof.

19. A compound represented by the formula: 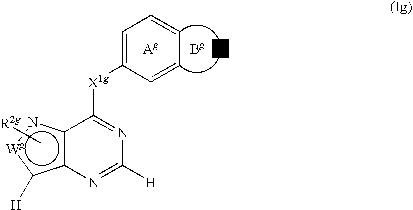
wherein
W^{g} is C(R^{1g}) or N,
ring A^{g} is an optionally substituted benzene ring,
ring B^{g} is an optionally substituted nitrogen-containing heterocycle,
X^{1g} is —NR^{3g}—Y^{1g}—, —O—, —S—, —SO—, —SO₂— or —CHR^{3g}—
wherein R^{3g} is a hydrogen atom or an optionally substituted aliphatic hydrocarbon group, or R^{3g} is optionally bonded to the carbon atom on the benzene ring for ring A^{g} to form an optionally substituted ring structure, and Y^{1g} is a bond, or a C₁₋₄alkylene or —O—(C₁₋₄ alkylene)-, each of which is optionally substituted, and
R^{1g} is a hydrogen atom, a halogen atom, or an optionally substituted group bonded via a carbon atom, a nitrogen atom or an oxygen atom,
R^{2g} is a hydrogen atom, or an optionally substituted group bonded via a carbon atom or a sulfur atom, or
R^{1g} and R^{2g}, or R^{2g} and R^{3g} are optionally bonded to form an optionally substituted ring structure,
or a salt thereof.

20. The compound of claim 19, which is a compound represented by the formula: 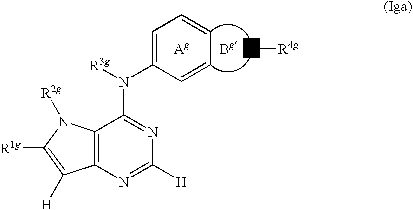
wherein R^{4g} is an optionally substituted hydrocarbon group, ring B^{g′} is a 5 or 6-membered nitrogen-containing heterocycle optionally further substituted besides R^{4g}, and the other symbols are as defined in claim 19.

21. The compound of claim 20, wherein
R^{1g} is a hydrogen atom, a halogen atom, a cyano group or an optionally halogenated C₁₋₆ alkyl group,
R^{2g} is a hydrogen atom or an optionally substituted C₁₋₆ alkyl group,
R^{3g} is a hydrogen atom or a C₁₋₆ alkyl group,
R^{4g} is (i) an optionally substituted C₆₋₁₄ aryl-C₁₋₈ alkyl group, (ii) an optionally substituted heterocyclyl-C₁₋₈ alkyl group, (iii) a C₁₋₈ alkyl group, or (iv) an optionally substituted C₆₋₁₄ aryl group.

22. The compound of claim 20, wherein
R^{1g} is a hydrogen atom, a halogen atom, a cyano group or an optionally halogenated C₁₋₆ alkyl group,
R^{2g} is
(i) a hydrogen atom,
(ii) a C₁₋₆ alkyl group, or
(iii) a C₁₋₆ alkyl group substituted by substituent(s) selected from the group consisting of
(a) —O—(CH₂)ₙ—OH,
(b) —NR^{5g}—CO—(CH₂)ₙ—OH,
(c) —NR^{5g}—CO—(CH₂)ₙ—SO₂-optionally halogenated C₁₋₄ alkyl,
(d) hydroxy, and
(e) amino
wherein n is an integer of 1 to 4, R^{5g} is a hydrogen atom or a C₁₋₄ alkyl group, and —(CH₂)ₙ— is optionally substituted by C₁₋₄ alkyl,
R^{3g} is a hydrogen atom or a C₁₋₆ alkyl group, 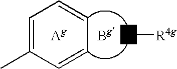
is the formula 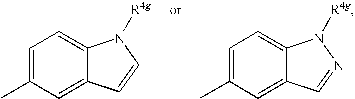
and
R^{4g} is (i) a C₆₋₁₄ aryl-C₁₋₈alkyl group optionally substituted by substituent(s) selected from the group consisting of halogen, C₁₋₆ alkyl-carbamoyl and halo C₁₋₆ alkoxy, (ii) an optionally substituted heterocyclyl-C₁₋₈ alkyl group, or (iii) an optionally substituted C₆₋₁₄ aryl group.

23. A compound selected from the following:
N-[2-(4-{[1-(3-fluorobenzyl)-1H-indazol-5-yl]amino}-5H-pyrrolo[3,2-d]pyrimidin-5-yl)ethyl]-2-(methylsulfonyl)acetamide,
N-[2-(4-{[1-(3-fluorobenzyl)-1H-indol-5-yl]amino}-5H-pyrrolo[3,2-d]pyrimidin-5-yl)ethyl]-3-hydroxy-3-methylbutanamide,
N-(tert-butyl)-3-[(5-{[5-(2-hydroxyethyl)-5H-pyrrolo[3,2-d]pyrimidin-4-yl]amino}-1H-indol-1-yl)methyl]benzamide,
N-(tert-butyl)-3-[(5-{[5-(2-hydroxyethyl)-5H-pyrrolo[3,2-d]pyrimidin-4-yl]amino}-1H-indazol-1-yl)methyl]benzamide, and
N-(tert-butyl)-6-[(5-{[5-(2-hydroxyethyl)-5H-pyrrolo[3,2-d]pyrimidin-4-yl]amino}-1H-indol-1-yl)methyl]pyridine-2-carboxamide,
or a salt thereof.

24. A compound represented by the formula: 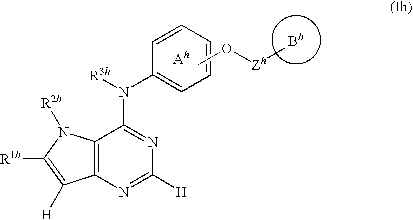
wherein
R^{1h} is a halogen atom or a halogenated C₁₋₆ alkyl group,
R^{2h} is a hydrogen atom, or an optionally substituted group bonded via a carbon atom or a sulfur atom, or
R^{1h} and R^{2h}, or R^{2h} and R^{3h} are bonded to form an optionally substituted ring structure,
R^{3h} is a hydrogen atom or an optionally substituted aliphatic hydrocarbon group, or R^{3h} is optionally bonded to the carbon atom on the adjacent benzene ring to form an optionally substituted ring structure,
Z^{h} is a bond or an optionally substituted C₁₋₃ alkylene,
ring A^{h} is an optionally substituted benzene ring, and
ring B^{h} is (i) an optionally substituted C₆₋₁₄ aryl group, (ii) an optionally substituted heterocyclic group, or (iii) an optionally substituted C₅₋₈ cycloalkyl group,
or a salt thereof.

25. The compound of claim 24, which is a compound represented by the formula: 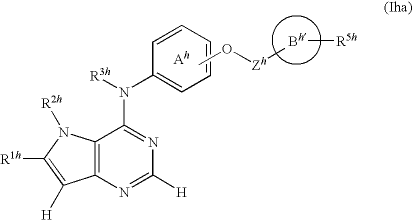
wherein
R^{5h} is
(i) an optionally substituted amino group,
(ii) an optionally substituted carbamoyl group,
(iii) an optionally substituted ureido group,
(iv) an optionally substituted sulfamoyl group,
(v) an optionally substituted heterocyclic group,
(vi) an optionally substituted hydrocarbon group,
(vii) a halogen atom, or
(viii) an optionally substituted carboxyl group, and
ring B^{h} is (i) a C₆₋₁₄ aryl group, (ii) a heterocyclic group, or (iii) a C₅₋₈ cycloalkyl group, each of which is optionally further substituted besides R^{5h}, and the other symbols are as defined in claim 24.

26. A compound selected from the following:
N-(3-{2-chloro-4-[(6-chloro-5-methyl-5H-pyrrolo[3,2-d]pyrimidin-4-yl)amino]phenoxy}phenyl)cyclopropanecarboxamide,
6-chloro-N-{3-chloro-4-[3-(trifluoromethyl)phenoxy]phenyl}-5-methyl-5H-pyrrolo[3,2-d]pyrimidine-4-amine,
N-[3-(2-chloro-4-{[6-chloro-5-(2-hydroxyethyl)-5H-pyrrolo[3,2-d]pyrimidin-4-yl]amino}phenoxy)phenyl]cyclopropanecarboxamide, and
N-(tert-butyl)-3-(2-chloro-4-{[6-chloro-5-(2-hydroxyethyl)-5H-pyrrolo[3,2-d]pyrimidin-4-yl]amino}phenoxy)benzamide,
or a salt thereof.

27. (canceled)

28. A pharmaceutical agent comprising claim 1 or a salt thereof.

29. The pharmaceutical agent of claim 28, which is a tyrosine kinase inhibitor.

30. The pharmaceutical agent of claim 28, which is an agent for the treatment of cancer.

31. The pharmaceutical agent of claim 30, wherein the cancer is breast cancer, ovarian cancer, colorectal cancer, gastric cancer, esophagus cancer, prostate cancer, lung cancer, pancreatic cancer or kidney cancer.

32. A method for the treatment of cancer in a mammal, which comprises administering an effective amount of the compound of claim 1 or a salt thereof, to the mammal.

33. (canceled)

34. The compound of claim 5, which is N-[2-(4-{[3-chloro-4-(3-chlorophenoxy)phenyl]amino}-5H-pyrrolo[3,2-d]pyrimidin-5-yl)ethyl]-2-methyl-2-(methylsulfonyl)propanamide or a salt thereof, or a hydrate thereof.

35. The compound of claim 5, which is N-[2-(4-{[3-chloro-4-(3-chlorophenoxy)phenyl]amino}-5H-pyrrolo[3,2-d]pyrimidin-5-yl)ethyl]-2-(ethylsulfonyl)acetamide or a salt thereof, or a hydrate thereof.

36. The compound of claim 5, which is N-[2-(4-{[3-chloro-4-(3-chlorophenoxy)phenyl]amino}-5H-pyrrolo[3,2-d]pyrimidin-5-yl)ethyl]-N,2-dimethyl-2-(methylsulfonyl)propanamide or a salt thereof, or a hydrate thereof.

37. The compound of claim 5, which is N-[2-(4-{[3-chloro-4-(3-methylphenoxy)phenyl]amino}-5H-pyrrolo[3,2-d]pyrimidin-5-yl)ethyl]-2-(methylsulfonyl)acetamide or a salt thereof, or a hydrate thereof.

38. The compound of claim 5, which is N-[2-(4-{[3-chloro-4-(3-fluorophenoxy)phenyl]amino}-5H-pyrrolo[3,2-d]pyrimidin-5-yl)ethyl]-2-(methylsulfonyl)acetamide or a salt thereof, or a hydrate thereof.

39. The compound of claim 5, which is N-[2-(4-{[4-(3-chlorophenoxy)-3-methylphenyl]amino}-5H-pyrrolo[3,2-d]pyrimidin-5-yl)ethyl]-2-methyl-2-(methylsulfonyl)propanamide, or a salt thereof, or a hydrate thereof.

40. The compound of claim 10, which is 2-{2-[4-({5-chloro-6-[3-(trifluoromethyl)phenoxy]pyridin-3-yl}amino)-5H-pyrrolo[3,2-d]pyrimidin-5-yl]ethoxy}ethanol or a salt thereof.

41. The compound of claim 10, which is N-{2-[4-({5-chloro-6-[3-(trifluoromethyl)phenoxy]pyridin-3-yl}amino)-5H-pyrrolo[3,2-d]pyrimidin-5-yl]ethyl}-2-(methylsulfonyl)acetamide or a salt thereof.

42. The compound of claim 10, which is N-{2-[4-({5-chloro-6-[3-(trifluoromethyl)phenoxy]pyridin-3-yl}amino)-5H-pyrrolo[3,2-d]pyrimidin-5-yl]ethyl}-3-hydroxy-3-methylbutanamide or a salt thereof.

43. The compound of claim 10, which is N-{2-[4-({5-chloro-6-[3-(trifluoromethoxy)phenoxy]pyridin-3-yl}amino)-5H-pyrrolo[3,2-d]pyrimidin-5-yl]ethyl}-2-(methylsulfonyl)acetamide or a salt thereof.

44. The compound of claim 10, which is N-(tert-butyl)-3-[(3-chloro-5-{[5-(2-hydroxyethyl)-5H-pyrrolo[3,2-d]pyrimidin-4-yl]amino}pyridin-2-yl)oxy]benzamide or a salt thereof.

45. The compound of claim 13, which is 2-{2-[4-({3-chloro-4-[3-(1,3-thiazol-5-yl)phenoxy]phenyl}amino)-5H-pyrrolo[3,2-d]pyrimidin-5-yl]ethoxy}ethanol or a salt thereof.

46. The compound of claim 13, which is N-(tert-butyl)-3-[2-chloro-4-({5-[2-(2-hydroxyethoxy)ethyl]-5H-pyrrolo[3,2-d]pyrimidin-4-yl}amino)phenoxy]benzamide or a salt thereof.

47. The compound of claim 13, which is 3-[2-chloro-4-({5-[2-(2-hydroxyethoxy)ethyl]-5H-pyrrolo[3,2-d]pyrimidin-4-yl}amino)phenoxy]-N-(2-hydroxy-1,1-dimethylethyl)benzamide or a salt thereof.

48. The compound of claim 13, which is N-(tert-butyl)-3-(2-chloro-4-{[5-(2-hydroxyethyl)-5H-pyrrolo[3,2-d]pyrimidin-4-yl]amino}phenoxy)benzamide or a salt thereof.

49. The compound of claim 13, which is N-(3-{2-chloro-4-[(6-cyano-5-methyl-5H-pyrrolo[3,2-d]pyrimidin-4-yl)amino]phenoxy}phenyl)cyclopropanecarboxamide or a salt thereof.

50. The compound of claim 13, which is N-(tert-butyl)-5-(2-chloro-4-{[5-(2-hydroxyethyl)-5H-pyrrolo[3,2-d]pyrimidin-4-yl]amino}phenoxy)-2-fluorobenzamide or a salt thereof.

51. The compound of claim 13, which is N-{2-[4-({3-chloro-4-[3-(dimethylamino)phenoxy]phenyl}amino)-5H-pyrrolo[3,2-d]pyrimidin-5-yl]ethyl}-3-hydroxy-3-methylbutanamide or a salt thereof.

52. The compound of claim 13, which is N-{2-[4-({3-chloro-4-[3-(dimethylamino)phenoxy]phenyl}amino)-5H-pyrrolo[3,2-d]pyrimidin-5-yl]ethyl}-2-(methylsulfonyl)acetamide or a salt thereof.

53. The compound of claim 13, which is N-(tert-butyl)-2-[3-(2-chloro-4-{[5-(2-hydroxyethyl)-5H-pyrrolo[3,2-d]pyrimidin-4-yl]amino}phenoxy)phenyl]acetamide or a salt thereof.

54. The compound of claim 13, which is N-{2-[4-({3-chloro-4-[3-(cyclopropylmethoxy)phenoxy]phenyl}amino)-5H-pyrrolo[3,2-d]pyrimidin-5-yl]ethyl}-2-(methylsulfonyl)acetamide or a salt thereof.

55. The compound of claim 13, which is N-{2-[4-({3-chloro-4-[3-(2,2-dimethylpropoxy)phenoxy]phenyl}amino)-5H-pyrrolo[3,2-d]pyrimidin-5-yl]ethyl}-2-(methylsulfonyl)acetamide or a salt thereof.

56. The compound of claim 13, which is 2-(methylsulfonyl)-N-{2-[4-({3-methyl-4-[3-(2,2,2-trifluoroethoxy)phenoxy]phenyl}amino)-5H-pyrrolo[3,2-d]pyrimidin-5-yl]ethyl}acetamide or a salt thereof.

57. The compound of claim 13, which is 2-[4-({3-chloro-4-[3-(isopropylsulfonyl)phenoxy]phenyl}amino)-5H-pyrrolo[3,2-d]pyrimidin-5-yl]ethanol or a salt thereof.

58. The compound of claim 13, which is N-[2-(4-{[3-chloro-4-(3-cyanophenoxy)phenyl]amino}-5H-pyrrolo[3,2-d]pyrimidin-5-yl)ethyl]-2-(methylsulfonyl)acetamide or a salt thereof.

59. The compound of claim 16, which is tert-butyl 4-{[2-chloro-4-({5-[2-(2-hydroxyethoxy)ethyl]-5H-pyrrolo[3,2-d]pyrimidin-4-yl}amino)phenoxy]methyl}piperidine-1-carboxylate or a salt thereof.

60. The compound of claim 16, which is tert-butyl 4-[(2-chloro-4-{[5-(2-hydroxyethyl)-5H-pyrrolo[3,2-d]pyrimidin-4-yl]amino}phenoxy)methyl]piperidine-1-carboxylate or a salt thereof.

61. The compound of claim 18, which is 2-[4-({3-chloro-4-[3-(1,1-difluoroethyl)phenoxy]phenyl}amino)-5H-pyrrolo[3,2-d]pyrimidin-5-yl]ethanol or a salt thereof.

62. The compound of claim 18, which is (1Z)-1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-one O-ethyloxime or a salt thereof.

63. The compound of claim 18, which is 1-{3-[2-chloro-4-({5-[2-(2-hydroxyethoxy)ethyl]-5H-pyrrolo[3,2-d]pyrimidin-4-yl}amino)phenoxy]phenyl}-2,2-dimethylpropan-1-ol or a salt thereof.

64. The compound of claim 18, which is 1-[3-(2-chloro-4-{[5-(2-hydroxyethyl)-5H-pyrrolo[3,2-d]pyrimidin-4-yl]amino}phenoxy)phenyl]-3,3-dimethylbutan-1-one or a salt thereof.

65. The compound of claim 18, which is N-(2-{4-[(3-methyl-4-{3-[(1E)-3-methylbut-1-en-1-yl]phenoxy}phenyl)amino]-5H-pyrrolo[3,2-d]pyrimidin-5-yl}ethyl)-2-(methylsulfonyl)acetamide or a salt thereof.

66. The compound of claim 18, which is N-{2-[4-({3-chloro-4-[3-(1- cyanocyclopropyl)phenoxy]phenyl}amino)-5H-pyrrolo[3,2-d]pyrimidin-5-yl]ethyl}-2-(methylsulfonyl)acetamide or a salt thereof.

67. The compound of claim 23, which is N-[2-(4-{[1-(3-fluorobenzyl)-1H-indazol-5-yl]amino}-5H-pyrrolo[3,2-d]pyrimidin-5-yl)ethyl]-2-(methylsulfonyl)acetamide or a salt thereof.

68. The compound of claim 23, which is N-[2-(4-{[1-(3-fluorobenzyl)-1H-indol-5-yl]amino}-5H-pyrrolo[3,2-d]pyrimidin-5-yl)ethyl]-3-hydroxy-3-methylbutanamide or a salt thereof.

69. The compound of claim 23, which is N-(tert-butyl)-3-[(5-{[5-(2-hydroxyethyl)-5H-pyrrolo[3,2-d]pyrimidin-4-yl]amino}-1H-indol-1-yl)methyl]benzamide or a salt thereof.

70. The compound of claim 23, which is N-(tert-butyl)-3-[(5-{[5-(2-hydroxyethyl)-5H-pyrrolo[3,2-d]pyrimidin-4-yl]amino}-1H-indazol-1-yl)methyl]benzamide or a salt thereof.

71. The compound of claim 23, which is N-(tert-butyl)-6-[(5-{[5-(2-hydroxyethyl)-5H-pyrrolo[3,2-d]pyrimidin-4-yl]amino}-1H-indol-1-yl)methyl]pyridine-2-carboxamide or a salt thereof.

72. The compound of claim 26, which is N-(3-{2-chloro-4-[(6-chloro-5-methyl-5H-pyrrolo[3,2-d]pyrimidin-4-yl)amino]phenoxy}phenyl)cyclopropanecarboxamide or a salt thereof.

73. The compound of claim 26, which is 6-chloro-N-{3-chloro-4-[3-(trifluoromethyl)phenoxy]phenyl}-5-methyl-5H-pyrrolo[3,2-d]pyrimidine-4-amine or a salt thereof.

74. The compound of claim 26, which is N-[3-(2-chloro-4-{[6-chloro-5-(2-hydroxyethyl)-5H-pyrrolo[3,2-d]pyrimidin-4-yl]amino}phenoxy)phenyl]cyclopropanecarboxamide or a salt thereof.

75. The compound of claim 26, which is N-(tert-butyl)-3-(2-chloro-4-{[6-chloro-5-(2-hydroxyethyl)-5H-pyrrolo[3,2-d]pyrimidin-4-yl]amino}phenoxy)benzamide or a salt thereof.


SEQUENCE LISTING (2 sequences)

SEQ ID NO: 1 - DNA, 66 bp, Artificial
  primer for cloning human HER2 gene
aattaagtcgacatggactacaaagacgatgacgacaagcgacggcagcagaagatccggaagtac

SEQ ID NO: 2 - DNA, 42 bp, Artificial
  primer for cloning human HER2 gene
aattaagcatgctcacactggcacgtccagacccaggtactc